(19) United States
(12) Patent Application Publication
(10) Pub. No.: US 2024/0101531 A1
(43) Pub. Date: Mar. 28, 2024

(54) PYRIDINAMINE DERIVATIVES AND THEIR USE AS POTASSIUM CHANNEL MODULATORS

(71) Applicant: Xenon Pharmaceuticals Inc., Burnaby (CA)

(72) Inventors: Paul Scott Charifson, Burnaby (CA); Christoph Martin Dehnhardt, Burnaby (CA); Julien A. Delbrouck, Burnaby (CA); Thilo Focken, Burnaby (CA); Wei Gong, Burnaby (CA); Shawn Johnstone, Burnaby (CA); Xiangyu Li, Burnaby (CA); Jia Yi Mo, Burnaby (CA); Juliette Sabbatani, Burnaby (CA); Hong Wang, Burnaby (CA); Steven Sigmund Wesolowski, Natick, MA (US); Alla Yurevna Zenova, Vancouver (CA); Wei Zhang, Burnaby (CA)

(21) Appl. No.: 18/330,100

(22) Filed: Jun. 6, 2023

Related U.S. Application Data
(60) Provisional application No. 63/350,180, filed on Jun. 8, 2022.

(51) Int. Cl. C07D 401/12; C07D 401/14; C07D 405/14; C07D 495/04; C07D 513/04; C07D 471/04; C07D 491/048; C07D 491/107; C07D 498/04; C07D 413/14; C07D 487/04; C07D 417/14; C07D 409/14; C07B 59/00; A61P 25/08

(52) U.S. Cl. CPC C07D 401/12; C07D 401/14; C07D 405/14; C07D 495/04; C07D 513/04; C07D 471/04; C07D 491/048; C07D 491/107; C07D 498/04; C07D 413/14; C07D 487/04; C07D 417/14; C07D 409/14; C07B 59/002; A61P 25/08; C07B 2200/05

(57) ABSTRACT

The present disclosure is directed to compounds of Formula (1):

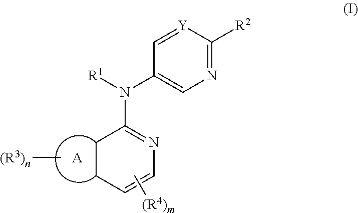

wherein m, n, Y, R¹, R², R³, R⁴ and R⁵ are each as described herein, as stereoisomers, enantiomers or tautomers thereof or mixtures thereof; or pharmaceutically acceptable salts, solvates or prodrugs thereof, and pharmaceutical compositions comprising the compounds of Formula (I), as described herein, which are useful as voltage-gated potassium channel modulators and are therefore are useful in treating seizure disorders such as epilepsy.

### TECHNICAL FIELD

This disclosure is directed to pyridinamine derivatives, as stereoisomers, enantiomers or tautomers thereof or mixtures thereof; or pharmaceutically acceptable salts, solvates or prodrugs thereof, and pharmaceutical compositions comprising the pyridinamine derivatives, which are useful as voltage-gated potassium channel allosteric modulators (“openers”) and are therefore useful in treating seizure disorders such as epilepsy.

### BACKGROUND

Epilepsy is a common seizure disorder, with a worldwide estimated prevalence of 0.7% of the population (50 million people) (see Hirtz, D. et al., Neurology. (2007), 68:326-337). It is characterized by abnormal electrical activities in the brain leading to seizures. For epidemiological purposes, the definition requires more than one unprovoked seizure of any type.

Patients with epilepsy have an increased mortality risk compared with the general population due primarily to the etiology of the disease. However, in patients with uncontrolled epilepsy, the greatest seizure-related risk of mortality is due to sudden unexpected death in epilepsy (SUDEP) (see, Hitiris, N. et al., Epilepsy and Behavior (2007), 10:363-376). Patients who participate in clinical trials of investigational antiepileptic drugs (AEDs) generally have had epilepsy for more than 10 years and have failed multiple AED therapies.

The pathophysiology of most forms of epilepsy remains poorly understood, but it is known that epileptic seizures arise from an excessively synchronous and sustained firing of a group of neurons. Persistent increase in neuronal excitability is common to all epileptic syndromes. The therapeutic strategy in treating epilepsy involves reducing neuronal excitability through various mechanistic pathways. Over the past two decades, several new AEDs were developed and marketed to expand the therapeutic spectrum by targeting different mechanisms of action and to improve the risk/benefit profile. Currently available AEDs are considered to act by inhibition of synaptic vesicle glycoprotein, potentiation of the inhibitory GABAergic neurotransmission, reduction of glutamate-mediated excitatory neurotransmission, or inhibition of voltage-gated sodium or calcium channels. Despite this, up to 30% of patients remain refractory to conventional treatment and continue to have uncontrolled seizures (see Brown, D. A. et al., Nature (1980), 283:673-676, and Elger, C. E. et al., Epilepsy Behav. (2008), 12:501-539). The quality of life in refractory patients is poor; they cannot drive a car, and they have difficulty working or living independently. Additionally, many patients have behavioral, neurological, and/or intellectual disturbances as sequelae of their seizure disorder. Current agents have minimal to no effects on neuronal potassium-gated channels, in spite of the fact that these channels have a major role in the control of neuronal excitability. Medicines with novel mechanisms of action, or medicines that improve on the already marketed AEDs are therefore needed to address the significant unmet clinical need for seizure control in patients with treatment-resistant epilepsy.

N-[4-(6-Fluoro-3,4-dihydro-1H-isoquinolin-2-yl)-2,6-dimethylphenyl]-3,3-dimethylbutanamide is a small molecule currently being developed for the treatment of seizure disorders, particularly for the treatment of partial onset (focal) seizures. This compound and its use as a potassium channel modulator is disclosed in U.S. Pat. Nos. 8,293,911 and 8,993,593, the disclosures of which are hereby incorporated by reference in their entireties.

Kv7.2/Kv7.3 underlie the neuronal “M-current”, named according to its initial characterization as a neuronal current decreased in response to muscarinic/cholinergic agonists (see Brown, D. A. et al., Nature (1980), 283:673-676). The M-current is a non-inactivating, hyperpolarizing current known to act as a brake on neuronal hyperexcitability. Consequently, a decrease in the Kv7.2-mediated M-current, for example through genetic loss-of-function, can cause neuronal depolarization and an increase in membrane and neuronal excitability that can lead to action potential bursts that manifest as epileptic seizures. In contrast, an increase in the Kv7.2-mediated M-current can hyperpolarize the cell membrane and thereby reduce neuronal excitability and prevent the initiation and propagation of action potential bursts and the resultant seizures. Enhancing the open state of Kv7.2/Kv7.3 channels in neurons favors a hyperpolarized resting state, which reduces rapid action potential spiking (i.e., burst firing). Such enhancement can provide a stabilizing effect on excitable, particularly hyper-excitable, neurons and therefore be useful in treating certain seizure disorders. This enhancement has been clinically proven to be effective for treatment of seizure disorders, such as partial onset seizures in adults with epilepsy, with retigabine/ezogabine, a known Kv7.2/Kv7.3 opener.

While significant advances have been made in this field, there remains a substantial need for compounds which are voltage-gated potassium channel allosteric modulators, thereby being useful in treating seizure disorders, preferably epilepsy, in a mammal, preferably a human.

### BRIEF SUMMARY

The present disclosure is directed to pyridinamine derivatives, as stereoisomers, enantiomers or tautomers thereof or mixtures thereof; or pharmaceutically acceptable salts, solvates or prodrugs thereof, and pharmaceutical compositions comprising the pyridinamine derivatives, which are useful as voltage-gated potassium channel allosteric modulators (“openers”) and are therefore useful in treating seizure disorders such as epilepsy.

Accordingly, in some embodiments, the present disclosure is directed to compounds of Formula (I):

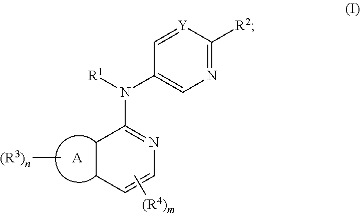

wherein:
m is 0 or 1;
n is 0, 1, 2 or 3;
Y is ═C(R⁵)— or ═N—;
[symbol] is a fused aryl or fused heteroaryl;
R¹ is hydrogen, alkyl, alkenyl, alkynyl, cycloalkyl, cycloalkylalkyl, aralkyl, heterocyclylalkyl or heteroarylalkyl;
R² is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
or R² and Y form a fused 5-membered cycloalkyl, a fused 5-membered heterocyclyl or a fused 5-membered heteroaryl;
each R³ is independently alkyl, alkenyl, alkynyl, halo, haloalkyl, haloalkenyl, —R⁹—OR⁶, —R⁹—N(R⁶)₂, —R⁹—C(O)R⁶, —R⁹—C(O)OR⁶, —R⁹—C(O)N(R⁶)₂, —N═S(O)(R⁷)R⁸, cycloalkyl, cycloalkylalkyl, aryl, aralkyl, heterocyclyl, heterocyclylalkyl, heteroaryl or heteroarylalkyl;
each R⁴ is independently —R⁹—OR⁶, —R⁹—N(R⁶)₂, —R⁹—C(O)R⁶, —R⁹—C(O)OR⁶, —R⁹—C(O)N(R⁶)₂, halo, alkyl, alkenyl, alkynyl, haloalkyl, haloalkenyl; cycloalkyl, cycloalkylalkyl, aryl, aralkyl, heterocyclyl, heterocyclylalkyl, heteroaryl or heteroarylalkyl;
R⁵ is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aralkyl, heterocyclylalkyl or heteroarylalkyl;
each R⁶ is independently hydrogen, —R¹⁰—OR¹¹, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond, a straight or branched alkylene chain or a straight or branched alkenylene chain;
R¹⁰ is a straight or branched alkylene chain or a straight or branched alkenylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl; as a stereoisomer, enantiomer, or tautomer thereof or a mixture thereof; or a pharmaceutically acceptable salt, solvate or prodrug thereof.

Accordingly, in some embodiments, the present disclosure is directed to compounds of Formula (I):

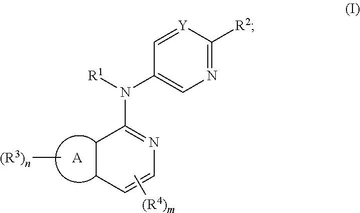

wherein:
m is 0 or 1;
n is 0, 1, 2 or 3;
Y is ═C(R⁵)— or ═N—;
[symbol] is a fused aryl or fused heteroaryl;
R¹ is hydrogen, alkyl, alkenyl, alkynyl, cycloalkyl, cycloalkylalkyl, aralkyl, heterocyclylalkyl or heteroarylalkyl;
R² is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloakylalkyl, aryl, aralkyl, heterocyclyl, heterocyclylalkyl, heteroaryl or heteroarylalkyl;
or R² and Y form a fused 5-membered cycloalkyl, a fused 5-membered heterocyclyl or a fused 5-membered heteroaryl;
each R³ is independently alkyl, alkenyl, alkynyl, halo, haloalkyl, haloalkenyl, cyanoalkyl, —R⁹—OR⁶, —R⁹—N(R⁶)₂, —R⁹—C(O)R⁶, —R⁹—C(O)OR⁶, —R⁹—C(O)N(R⁶)₂, —N═S(O)(R⁷)R⁸, cycloalkyl, cycloakylalkyl, aryl, aralkyl, heterocyclyl, heterocyclylalkyl, heteroaryl or heteroarylalkyl;
each R⁴ is independently —R⁹—OR⁶, —R⁹—N(R⁶)₂, —R⁹—C(O)R⁶, —R⁹—C(O)OR⁶, —R⁹—C(O)N(R⁶)₂, halo, alkyl, alkenyl, alkynyl, haloalkyl, haloalkenyl; cycloalkyl, cycloakylalkyl, aryl, aralkyl, heterocyclyl, heterocyclylalkyl, heteroaryl or heteroarylalkyl;
R⁵ is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aralkyl, heterocyclylalkyl or heteroarylalkyl;
each R⁶ is independently hydrogen, —R¹⁰—OR¹¹, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond, a straight or branched alkylene chain or a straight or branched alkenylene chain;
R¹⁰ is a straight or branched alkylene chain or a straight or branched alkenylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof. In other embodiments, this disclosure is directed to pharmaceutical compositions comprising a pharmaceutically acceptable excipient and a compound of Formula (I), as a stereoisomer, enantiomer, or tautomer thereof or a mixture thereof; or a pharmaceutically acceptable salt, solvate or prodrug thereof, as described above.

In other embodiments, this disclosure is directed to methods of treating a disease or condition in a mammal modulated by a voltage-gated potassium channel, wherein the methods comprise administering to a mammal in need thereof a therapeutically effective amount of a compound of Formula (I), as a stereoisomer, enantiomer, or tautomer thereof or a mixture thereof; or a pharmaceutically acceptable salt, solvate or prodrug thereof, as described above.

In other embodiments, this disclosure is directed to methods for the treatment of epilepsy and/or epileptic seizure disorder in a mammal, preferably a human, wherein the methods comprise administering to the mammal in need thereof a therapeutically effective amount of a compound of Formula (I), as set forth above, as a stereoisomer, enantiomer or tautomer thereof or mixtures thereof; or a pharmaceutically acceptable salt, solvate or prodrug thereof, or a pharmaceutical composition comprising a therapeutically effective amount of a compound of Formula (I), as set forth above, as a stereoisomer, enantiomer or tautomer thereof or mixtures thereof, or a pharmaceutically acceptable salt, solvate or prodrug thereof, and a pharmaceutically acceptable excipient.

In other embodiments, this disclosure is directed to methods of preparing a compound of Formula (I), as set forth above, as a stereoisomer, enantiomer or tautomer thereof or mixtures thereof; or a pharmaceutically acceptable salt, solvate or prodrug thereof, or a pharmaceutical composition comprising a therapeutically effective amount of a compound of Formula (I), as set forth above, as a stereoisomer, enantiomer or tautomer thereof or mixtures thereof, or a pharmaceutically acceptable salt, solvate or prodrug thereof, and a pharmaceutically acceptable excipient.

In other embodiments, this disclosure is directed to pharmaceutical therapy in combination with one or more other compounds of Formula (I) or one or more other accepted therapies or as any combination thereof to increase the potency of an existing or future drug therapy or to decrease the adverse events associated with the accepted therapy. In one embodiment, this disclosure is directed to a pharmaceutical composition combining a compound of Formula (I) with established or future therapies for the indications listed herein.

### DETAILED DESCRIPTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Compound of the disclosure” or “compounds of the disclosure” refer to compounds of Formula (I), as described above in the Brief Summary, as stereoisomers, enantiomers, or tautomers thereof or mixtures thereof; or pharmaceutically acceptable salts, solvates or prodrugs thereof.

“Present disclosure” refers to this entire disclosure.

“Amino” refers to the —NH₂ radical.

“Cyano” refers to the —CN radical.

“Hydroxy” refers to the —OH radical.

“Imino” refers to the ═NH substituent.

“Nitro” refers to the —NO₂ radical.

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms, preferably one to eight carbon atoms and which is attached to the rest of the molecule by a single bond, e.g., ethynyl, propynyl, butynyl, pentynyl, hexynyl, and the like. Unless stated otherwise specifically in the specification, an alkynyl group is optionally substituted by one or more of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), or —S(O)ₜN(R²⁰)₂ (where t is 1 to 2), where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Aryl” refers to a hydrocarbon ring system radical comprising hydrogen, 6 to 18 carbon atoms and at least one aromatic ring. For purposes of this disclosure, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted by one or more substituents independently selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{c} where R_{b} is an alkylene chain as defined above and R, is one or more aryl radicals as defined above. In some embodiments, an aralkyl is benzyl, diphenylmethyl, and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R_{d}—R_{c} where R_{d} is an alkenylene chain as defined above and R, is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Fused” refers to any ring system described herein which is fused to an existing ring structure in the compounds of the disclosure. When the fused ring system is a heterocyclyl or a heteroaryl, any carbon in the existing ring structure which becomes part of the fused ring system may be replaced with a nitrogen.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Cyanoalkyl” refers to an alkyl radical, as defined above, that is substituted by one or more cyano radicals, as defined above. The alkyl part of the cyanoalkyl radical may be optionally substituted as defined above for an alkyl group.

“Alkoxyalkyl” refers to a radical with the following formula: —R^{a}OR^{b} wherein R^{a} is a straight or branched alkylene chain as defined herein and R^{b} is an alkyl radical as defined above. Both the alkyl and alkylene part of the alkoxyalkyl radical may be optionally substituted as defined above for an alkyl or an alkylene group, respectively.

“Heterocyclyl” refers to a stable 3- to 18-membered non-aromatic ring radical which consists of two to twelve carbon atoms and from one to six heteroatoms selected from the group consisting of nitrogen, oxygen, and sulfur. Unless stated otherwise specifically in the specification, the heterocyclyl radical may be a monocyclic, bicyclic, tricyclic, or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, dioxinyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, alkenyl, haloalkyl, cycloalkyl, cycloalkylalkyl, aryl, aralkyl, heterocyclyl, heterocyclylalkyl, heteroaryl or heteroarylalkyl.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkyene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen, and sulfur, and at least one aromatic ring. For purposes of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e., thienyl). Unless stated otherwise specifically in the specification, a heteroaryl group may be optionally substituted by one or more substituents selected from the group consisting of alkyl, alkenyl, halo, haloalkyl, haloalkenyl, cyano, oxo, thioxo, nitro, thioxo, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen. An N-heteroaryl radical may be optionally substituted as described above for heteroaryl radicals.

“O-heteroaryl” refers to a heteroaryl radical as defined above containing at least one oxygen atom and no nitrogen atom. An O-heteroaryl radical may be optionally substituted as described above for heteroaryl radicals.

“S-heteroaryl” refers to a heteroaryl radical as defined above containing at least one sulfur atom and no nitrogen atom. An S-heteroaryl radical may be optionally substituted as described above for heteroaryl radicals.

“S,N-heteroaryl” refers to a N-heteroaryl radical as defined above containing at least one sulfur atom and at least one nitrogen atom. An S,N-heteroaryl radical may be optionally substituted as described above for N-heteroaryl radicals.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Prodrugs” is meant to indicate a compound that may be converted under physiological conditions or by solvolysis to a biologically active compound of the disclosure. Thus, the term “prodrug” refers to a metabolic precursor of a compound of the disclosure that is pharmaceutically acceptable. A prodrug may be inactive when administered to a subject in need thereof but is converted in vivo to an active compound of the disclosure. Prodrugs are typically rapidly transformed in vivo to yield the parent compound of the disclosure, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the disclosure in vivo when such prodrug is administered to a mammalian subject. Prodrugs of a compound of the disclosure may be prepared by modifying functional groups present in the compound of the disclosure in such a way that the modifications are cleaved, either in routine manipulation or in vivo, to the parent compound of the disclosure. Prodrugs include compounds of the disclosure wherein a hydroxy, amino or mercapto group is bonded to any group that, when the prodrug of the compound of the disclosure is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the disclosure, and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g., cats, dogs, swine, cattle, sheep, goats, horses, rabbits), and non-domestic animals such as wildlife, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution (“unsubstituted”). When a functional group is described as “optionally substituted,” and in turn, substituents on the functional group are also “optionally substituted” and so on, for the purposes of this disclosure, such iterations are limited to five, preferably such iterations are limited to two. In some embodiments, such iterations are limited to one.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid, and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts, and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like. Particularly preferred organic bases are isopropylamine, diethylamine, ethanolamine, trimethylamine, dicyclohexylamine, choline, and caffeine.

Often crystallizations produce a solvate of the compound of the disclosure. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate, and the like, as well as the corresponding solvated forms. The compound of the disclosure may be true solvates, while in other cases; the compound of the disclosure may merely retain adventitious water or be a mixture of water plus some adventitious solvent.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents, or excipients therefor.

“Seizure disorders” refers to seizures and disorders associated with seizures such as partial onset (focal) seizures, photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia, and paroxysmal dyskinesia.

Preferably, the term “seizure disorder” refers to partial onset (focal) epilepsy. “Therapeutically effective amount” refers to a range of amounts of a compound of the disclosure, which, upon administration to a human, treats, ameliorates, or prevents a seizure disorder, preferably epilepsy, in the human, or exhibits a detectable therapeutic or preventative effect in the human having a seizure disorder. The effect is detected by, for example, a reduction in seizures (frequency) or by the severity of seizures (quality). The precise therapeutically effective amount for a given human will depend upon the human's size and health, the nature and extent of the seizure disorder, the presence of any concomitant medications, and other variables known to those of skill in the art. The therapeutically effective amount for a given situation is determined by routine experimentation and is within the judgment of the clinician.

“Treatment” refers to therapeutic applications to slow or stop progression of a seizure disorder, prophylactic application to prevent development of a seizure disorder, and/or reversal of a seizure disorder. Reversal of a seizure disorder differs from a therapeutic application which slows or stops a seizure disorder in that with a method of reversing, not only is progression of a seizure disorder completely stopped, but cellular behavior is moved to some degree toward a normal state that would be observed in the absence of the seizure disorder.

“Treating” or “treatment” as used herein covers the treatment of the disease or condition of interest in a mammal, preferably a human, having the disease or condition of interest, and includes:
(a) preventing the disease or condition from occurring in a mammal, in particular, when such mammal is predisposed to the condition but has not yet been diagnosed as having it;
(b) inhibiting the disease or condition, i.e., arresting its development;
(c) relieving (or ameliorating) the disease or condition, i.e., causing regression of the disease or condition; or
(d) relieving (or ameliorating) the symptoms resulting from the disease or condition, i.e., relieving pain without addressing the underlying disease or condition.

As used herein, the terms “disease” and “condition” may be used interchangeably or may be different in that the malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of this disclosure may contain at least one asymmetric carbon atom and thus may exist as racemates, enantiomers and/or diastereoisomers. For the present disclosure, the words diastereomer and diastereoisomer and related terms are equivalent and interchangeable. Unless otherwise indicated, this disclosure includes all enantiomeric and diastereoisomeric forms of the compounds of Formula (I). Pure stereoisomers, mixtures of enantiomers and/or diastereoisomers, and mixtures of different compounds of the disclosure are included within this disclosure. Thus, compounds of Formula (I) may occur as racemates, racemic or diastereoisomeric mixtures and as individual diastereoisomers, or enantiomers, unless a specific stereoisomer enantiomer or diastereoisomer is identified, with all isomeric forms being included in the present disclosure. For this disclosure, a racemate or racemic mixture implies a 50:50 mixture of stereoisomers only. Other enantiomerically or diastereomerically enriched mixtures of varying ratios of stereoisomers are also contemplated.

“Enantiomers” refer to asymmetric molecules that can exist in two different isomeric forms which have different configurations in space. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane-polarized light in different directions). Because they do not have a plane of symmetry, enantiomers are not identical with their mirror images; molecules which exist in two enantiomeric forms are chiral, which means that they can be regarded as occurring in “left” and “right” handed forms. The most common cause of chirality in organic molecules is the presence of a tetrahedral carbon bonded to four different substituents or groups. Such a carbon is referred to as a chiral center, or stereogenic center.

Enantiomers have the same empirical chemical formula, and are generally chemically identical in their reactions, their physical properties, and their spectroscopic properties. However, enantiomers show different chemical reactivity toward other asymmetric compounds, and respond differently toward asymmetric physical disturbances. The most common asymmetric disturbance is polarized light.

An enantiomer can rotate plane-polarized light; thus, an enantiomer is optically active. Two different enantiomers of the same compound will rotate plane-polarized light in the opposite direction; thus, the light can be rotated to the left or counterclockwise for a hypothetical observer (this is levarotatory or “l”, or minus or “−”) or it can be rotated to the right or clockwise (this is dextrorotatory or “d” or plus or “+”). The sign of optical rotation (+) or (−), is not related to the R, S designation. A mixture of equal amounts of two chiral enantiomers is called a racemic mixture, or racemate, and is denoted either by the symbol (+/−) or by the prefix “d,l” to indicate a mixture of dextrorotatory and levorotatory forms. Racemates or racemic mixtures show zero optical rotation because equal amounts of the (+) and (−) forms are present. In general, the presence of a single enantiomer rotates polarized light in only one direction; thus, a single enantiomer is referred to as optically pure.

The designations “R” and “S” are used to denote the three-dimensional arrangement of atoms (or the configuration) of the stereogenic center. The designations may appear as a prefix or as a suffix; they may or may not be separated from the enantiomer name by a hyphen; they may or may not be hyphenated; and they may or may not be surrounded by parentheses. A method for determining the designation is to refer to the arrangement of the priority of the groups at the stereogenic center when the lowest priority group is oriented away from a hypothetical observer: If the arrangement of the remaining three groups from the higher to the lower priority is clockwise, the stereogenic center has an “R” configuration; if the arrangement is counterclockwise, the stereogenic center has an “S” configuration.

“Resolution” or “resolving” when used in reference to a racemic compound or mixture refers to the separation of a racemate into its two enantiomeric forms (i.e., (+) and (−); (R) and (S) forms).

“Enantiomeric excess” or “ee” refers to a product wherein one enantiomer is present in excess of the other and is defined as the absolute difference in the mole fraction of each enantiomer. Enantiomeric excess is typically expressed as a percentage of an enantiomer present in a mixture relative to the other enantiomer. For purposes of this disclosure, the (S)-enantiomer of a compound prepared by the methods disclosed herein is considered to be “substantially free” of the corresponding (R)-enantiomer when the (S)-enantiomer is present in enantiomeric excess of greater than 80%, preferably greater than 90%, more preferably greater than 95% and most preferably greater than 99%.

A “tautomer” refers to a proton shift from one atom of a molecule to another atom of the same molecule. The present disclosure includes tautomers of any compound of Formula (I) as described herein.

The use of parentheses and brackets in substituent groups may be used herein to conserve space. Accordingly, the use of parenthesis in a substituent group indicates that the group enclosed within the parentheses is attached directly to the atom preceding the parenthesis. The use of brackets in a substituent group indicates that the group enclosed within the brackets is also attached directly to the atom preceding the parenthesis.

For example, a compound of Formula (I) wherein n is 1, m is 0, [symbol] is a fused phenyl, R¹ is hydrogen, R² is chloro and R³ is difluoromethoxy, i.e., a compound of the following structure:

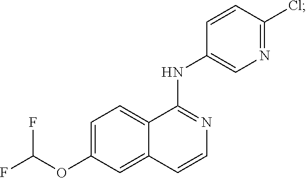 [SMILES: FC(F)Oc1ccc2c(Nc3ccc(Cl)nc3)nccc2c1]

is named herein as N-(6-chloropyridin-3-yl)-6-(difluoromethoxy)isoquinolin-1-amine.

### EMBODIMENTS

One embodiment of the disclosure provides compounds of Formula (I) as set forth above in the Brief Summary, as individual stereoisomers, enantiomers or tautomers thereof or as mixtures thereof; or pharmaceutically acceptable salts, solvates or prodrugs thereof.

In some embodiments, R¹ is optionally substituted when R¹ is alkyl. In certain embodiments, R¹ is optionally substituted with —O—CH₂CH₂—Si(CH₃)₃ when R¹ is alkyl (e.g., methyl).

In some embodiments, R³ is optionally substituted with one or more substituents selected from the group consisting of halo, alkyl, alkynyl, cycloalkyl, haloalkyl, cyano, cyanoalkyl, oxo (i.e., ═O), —C(═O)NH₂, —OH, alkoxy (e.g., methoxy), alkoxyalkyl (e.g., methoxymethyl), aralkyl (e.g., benzyl), heteroarylalkyl, heteroarylalkoxy, —C(═O)O-alkyl (e.g., —C(═O)O—CH₂CH₃), —C(═O)-alkyl (e.g., —C(═O)CH₃), —C(═O)-cycloalkyl (e.g., —C(═O)cyclopropyl), —S(O)₂-alkyl (e.g., —S(O)₂CH₃), —NH—C(═O)-alkyl, —NH—C(═O)-haloalkyl, —NH—C(═O)-heteroaryl (e.g., methylthiazolyl), —NH₂, —C(═O)N(CH₃)₂, —S(O)₂NH₂, heterocyclyl, hydroxyalkyl (e.g., —CH₂OH), —NH—C(═O)O-alkyl, ═NH, and deuterium.

In some embodiments, an occurrence of R⁶ (e.g., when R⁶ is —R¹⁰—OR¹¹, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl) is optionally substituted with one or more substituents selected from the group consisting of halo, alkyl, alkynyl, cycloalkyl, haloalkyl, cyano, cyanoalkyl, oxo (i.e., ═O), —C(═O)NH₂, —OH, alkoxy (e.g., methoxy), alkoxyalkyl (e.g., methoxymethyl), aralkyl (e.g., benzyl), heteroarylalkyl, heteroarylalkoxy, —C(═O)O-alkyl (e.g., —C(═O)O—CH₂CH₃), —C(═O)-alkyl (e.g., —C(═O)CH₃), —C(═O)-cycloalkyl (e.g., —C(═O)cyclopropyl), —S(O)₂-alkyl (e.g., —S(O)₂CH₃), —NH—C(═O)-alkyl, —NH—C(═O)-haloalkyl, —NH—C(═O)-heteroaryl (e.g., methylthiazolyl), —NH₂, —C(═O)N(CH₃)₂, —S(O)₂NH₂, heterocyclyl, hydroxyalkyl (e.g., —CH₂OH), —NH—C(═O)O-alkyl, ═NH, and deuterium.

One embodiment of a compound of Formula (I), as described above in the Brief Summary, is a compound of Formula (I) wherein [symbol] is a fused aryl and m, n, Y, R¹, R², R³ and R⁴ are each as described above in the Brief Summary; as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein [symbol] is a fused phenyl having the following formula (Ia):

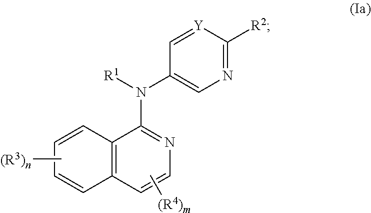

wherein m, n, Y, R¹, R², R³ and R⁴ are each as described above in the Brief Summary; as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein Y is ═C(R⁵)— having the formula (Ia1):

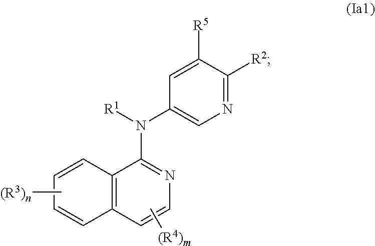

wherein m, n, R¹, R², R³, R⁴ and R⁵ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1, 2 or 3;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1, 2 or 3;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl, —R⁹—OR⁶, heterocyclyl, or heterocyclylalkyl;
each R⁹ is independently a direct bond;
R¹⁰ is a straight or branched alkylene chain; and
R¹¹ is hydrogen, alkyl, alkoxyalkyl, or haloalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) selected from the following:
N-(6-chloropyridin-3-yl)-6-(cyclopropylmethoxy)isoquinolin-1-amine;
N-(6-chloropyridin-3-yl)-6-(pyridin-2-ylmethoxy)isoquinolin-1-amine;
N-(6-chloropyridin-3-yl)-6-(2-methoxyethoxy)isoquinolin-1-amine;
N-(6-chloropyridin-3-yl)-6-isopropoxyisoquinolin-1-amine;
N-(6-chloropyridin-3-yl)-6-fluoroisoquinolin-1-amine;
N-(6-chloropyridin-3-yl)isoquinolin-1-amine;
N-(6-chloropyridin-3-yl)-6-(difluoromethoxy)isoquinolin-1-amine;
N-(6-chloropyridin-3-yl)-6-methoxyisoquinolin-1-amine;
N-(6-chloropyridin-3-yl)-6-propoxyisoquinolin-1-amine;
N-(6-chloropyridin-3-yl)-6-methylisoquinolin-1-amine;
N-(6-chloropyridin-3-yl)-6-phenoxyisoquinolin-1-amine;
N-(6-chloropyridin-3-yl)-6-((1-(trifluoromethyl)cyclopropyl)methoxy)isoquinolin-1-amine;
N-(6-chloropyridin-3-yl)-6-(cyclopropylmethoxy)-5-fluoroisoquinolin-1-amine;
N-(6-chloropyridin-3-yl)-6-(1-cyclopropylethoxy)isoquinolin-1-amine;
N¹-(6-chloropyridin-3-yl)-N⁶-(cyclopropylmethyl)isoquinoline-1,6-diamine;
Methyl 1-((6-chloropyridin-3-yl)amino)isoquinoline-6-carboxylate;
((1-((6-chloropyridin-3-yl)amino)isoquinolin-6-yl)imino)dimethyl-λ⁶-sulfanone;
6-(cyclopropylmethoxy)-N-(6-methylpyridin-3-yl)isoquinolin-1-amine;
N-(6-chloropyridin-3-yl)-6-((5,5-dimethyltetrahydrofuran-2-yl)methoxy)isoquinolin-1-amine;
N-(6-chloropyridin-3-yl)-6-(pyridin-2-ylmethoxy)isoquinolin-1-amine;
N-(6-chloropyridin-3-yl)-6-(2-methoxyethoxy)isoquinolin-1-amine;
N-(6-chloropyridin-3-yl)-5-fluoroisoquinolin-1-amine;
N-(6-chloro-5-methoxypyridin-3-yl)-6-((3-methyloxetan-3-yl)methoxy)isoquinolin-1-amine;
N-(6-chloropyridin-3-yl)-6-(2-cyclopropoxyethoxy)isoquinolin-1-amine;
2-chloro-5-((6-((3-methyloxetan-3-yl)methoxy)isoquinolin-1-yl)amino)pyridin-3-ol;
N-(6-(difluoromethyl)pyridin-3-yl)-6-((1-fluorocyclopropyl)methoxy)isoquinolin-1-amine;
N-(5-chloro-6-methylpyridin-3-yl)-6-((1-fluorocyclopropyl)methoxy)isoquinolin-1-amine;
6-((1-fluorocyclopropyl)methoxy)-N-(6-(trifluoromethyl)pyridin-3-yl)isoquinolin-1-amine;
N-(5-chloropyridin-3-yl)-6-((1-fluorocyclopropyl)methoxy)isoquinolin-1-amine;
4-(((1-((6-chloropyridin-3-yl)amino)isoquinolin-6-yl)oxy)methyl)tetrahydro-2H-pyran-4-carbonitrile;
N-(6-chloropyridin-3-yl)-6-(pyrimidin-4-ylmethoxy)isoquinolin-1-amine;
N-(6-chloropyridin-3-yl)-6-((3-fluorooxetan-3-yl)methoxy)isoquinolin-1-amine;
5-(((1-((6-chloropyridin-3-yl)amino)isoquinolin-6-yl)oxy)methyl)-1-methylpyrrolidin-2-one;
N-(6-chloro-5-methoxypyridin-3-yl)-6-((1-methyl-1H-pyrazol-4-yl)methoxy)isoquinolin-1-amine;
N-(5-methoxy-6-methylpyridin-3-yl)-6-((1-methyl-1H-pyrazol-4-yl)methoxy)isoquinolin-1-amine;
N-(6-chloropyridin-3-yl)-6-((3-fluoroazetidin-3-yl)methoxy)isoquinolin-1-amine;
3-(((1-((6-chloropyridin-3-yl)amino)isoquinolin-6-yl)oxy)methyl)-1-methyl-1H-pyrazole-5-carbonitrile;
N-(6-chloropyridin-3-yl)-6-((5-methyl-1,3,4-oxadiazol-2-yl)methoxy)isoquinolin-1-amine;
N-(6-chloropyridin-3-yl)-6-((1-methyl-1H-imidazol-5-yl)methoxy)isoquinolin-1-amine;
N-(6-methoxypyridin-3-yl)-6-((3-methyloxetan-3-yl)methoxy)isoquinolin-1-amine;
1-((6-chloropyridin-3-yl)amino)isoquinolin-6-ol;
6-(2-(1-oxa-6-azaspiro[3.3]heptan-6-yl)ethoxy)-N-(6-chloropyridin-3-yl)isoquinolin-1-amine;
6-(2-(2-azaspiro[3.3]heptan-2-yl)ethoxy)-N-(6-chloropyridin-3-yl)isoquinolin-1-amine;
N-(6-chloropyridin-3-yl)-6-(2-(3-methoxyazetidin-1-yl)ethoxy)isoquinolin-1-amine;
6-(2-(2-oxa-6-azaspiro[3.3]heptan-6-yl)ethoxy)-N-(6-chloropyridin-3-yl)isoquinolin-1-amine;
6-(2-(1H-imidazol-1-yl)ethoxy)-N-(6-chloropyridin-3-yl)isoquinolin-1-amine;
2-(2-((1-((6-chloropyridin-3-yl)amino)isoquinolin-6-yl)oxy)ethyl)-1,2-dihydro-3H-pyrazol-3-one;
N-(6-chloropyridin-3-yl)-6-fluoro-N-((2-(trimethylsilyl)ethoxy)-methyl)isoquinolin-1-amine;
1-(((1-((6-chloropyridin-3-yl)amino)isoquinolin-6-yl)oxy)methyl)cyclobutane-1-carbonitrile;
3-((1-((6-chloropyridin-3-yl)amino)isoquinolin-6-yl)oxy)-2,2-dimethylpropanenitrile;
rac-(3R,4S)-4-((1-((6-chloropyridin-3-yl)amino)isoquinolin-6-yl)oxy)tetrahydrofuran-3-ol;
1-(((1-((6-chloropyridin-3-yl)amino)isoquinolin-6-yl)oxy)methyl)cyclopropane-1-carbonitrile;
6-((2-oxaspiro[3.3]heptan-6-yl)methoxy)-N-(6-chloropyridin-3-yl)isoquinolin-1-amine;
6-((1H-pyrazol-1-yl)methoxy)-N-(6-chloropyridin-3-yl)isoquinolin-1-amine;
N-(6-chloropyridin-3-yl)-6-((3,3-difluorocyclohexyl)oxy)isoquinolin-1-amine;
N-(6-chloropyridin-3-yl)-6-((6,7-dihydro-5H-pyrazolo[5,1-b][1,3]oxazin-3-yl)methoxy)isoquinolin-1-amine;
3-(((1-((6-chloropyridin-3-yl)amino)isoquinolin-6-yl)oxy)methyl)oxetane-3-carbonitrile;
N-(6-chloropyridin-3-yl)-6-((4,4-difluorotetrahydrofuran-3-yl)oxy)isoquinolin-1-amine 2,2,2-trifluoroacetate;
N-(6-chloropyridin-3-yl)-6-((4-methyl-4H-1,2,4-triazol-3-yl)methoxy)isoquinolin-1-amine;
N-(6-chloropyridin-3-yl)-6-(2-(oxetan-3-yl)ethoxy)isoquinolin-1-amine;
6-((1-benzylpiperidin-4-yl)oxy)-N-(6-chloropyridin-3-yl)isoquinolin-1-amine;
N-(6-chloropyridin-3-yl)-6-(1-(1-fluorocyclopropyl)ethoxy)isoquinolin-1-amine;
N-(6-chloropyridin-3-yl)-6-fluoro-N-((2-(trimethylsilyl)ethoxy)-methyl)isoquinolin-1-amine;
N-(6-chloropyridin-3-yl)-6-((4,4-difluorocyclohexyl)oxy)isoquinolin-1-amine;
N-(6-chloropyridin-3-yl)-6-(pyrazin-2-ylmethoxy)isoquinolin-1-amine;
rac-(1R,3S)-3-((1-((6-chloropyridin-3-yl)amino)isoquinolin-6-yl)oxy)cyclohexan-1-ol;
(1R,3R)-3-((1-((6-chloropyridin-3-yl)amino)isoquinolin-6-yl)oxy)cyclobutan-1-ol;
6-((8-benzyl-8-azabicyclo[3.2.1]octan-3-yl)oxy)-N-(6-chloropyridin-3-yl)isoquinolin-1-amine;
N-(6-chloropyridin-3-yl)-6-(2-(3-methyloxetan-3-yl)ethoxy)isoquinolin-1-amine;
N-(6-chloropyridin-3-yl)-6-(2-(1-methyl-1H-pyrazol-4-yl)ethoxy)isoquinolin-1-amine;
Cis-4-((1-((6-chloropyridin-3-yl)amino)isoquinolin-6-yl)oxy)cyclohexan-1-ol;
N-(6-chloropyridin-3-yl)-6-(2-methyl-2-morpholinopropoxy)isoquinolin-1-amine;
rac-(1 S,3R)-3-((1-((6-chloropyridin-3-yl)amino)isoquinolin-6-yl)oxy)cyclopentan-1-ol;
rac-(1R,3R)-3-((1-((6-chloropyridin-3-yl)amino)isoquinolin-6-yl)oxy)cyclopentan-1-ol;
N-(6-chloropyridin-3-yl)-6-(((1s,4s)-4-methoxycyclohexyl)oxy)isoquinolin-1-amine;
N-(6-chloropyridin-3-yl)-6-(((1r,4r)-4-methoxycyclohexyl)oxy)isoquinolin-1-amine;
N-(6-chloropyridin-3-yl)-6-((1-(pyridin-3-yl)propan-2-yl)oxy)isoquinolin-1-amine;
3-(2-((1-((6-chloropyridin-3-yl)amino)isoquinolin-6-yl)oxy)ethyl)oxazolidin-2-one;
(R)-6-((1-benzylpiperidin-3-yl)oxy)-N-(6-chloropyridin-3-yl)isoquinolin-1-amine;
N-(6-chloropyridin-3-yl)-6-((1-morpholinopropan-2-yl)oxy)isoquinolin-1-amine;
ethyl 1-(((1-((6-chloropyridin-3-yl)amino)isoquinolin-6-yl)oxy)methyl)cyclopropane-1-carboxylate;
(S)-1-(3-((1-((6-chloropyridin-3-yl)amino)isoquinolin-6-yl)oxy)pyrrolidin-1-yl)ethan-1-one;
(R)-1-(3-((1-((6-chloropyridin-3-yl)amino)isoquinolin-6-yl)oxy)pyrrolidin-1-yl)ethan-1-one;
6-((2-oxaspiro[3.3]heptan-6-yl)oxy)-N-(6-chloropyridin-3-yl)isoquinolin-1-amine;
6-((1-oxaspiro[3.3]heptan-6-yl)methoxy)-N-(6-chloropyridin-3-yl)isoquinolin-1-amine;
N-(6-chloropyridin-3-yl)-6-((tetrahydro-1H-pyrrolizin-7a(5H)-yl)methoxy)isoquinolin-1-amine;
N-(6-chloropyridin-3-yl)-6-(2-(3-fluoroazetidin-1-yl)ethoxy)isoquinolin-1-amine;
N-(6-chloropyridin-3-yl)-6-((1-(oxetan-3-yl)-1H-pyrazol-4-yl)methoxy)isoquinolin-1-amine;
N-(6-chloropyridin-3-yl)-6-((1-(methylsulfonyl)piperidin-4-yl)oxy)isoquinolin-1-amine;
N-(6-chloropyridin-3-yl)-6-((1-(2-methoxyethyl)piperidin-4-yl)oxy)isoquinolin-1-amine;
6-(2-(1H-pyrazol-1-yl)ethoxy)-N-(6-chloropyridin-3-yl)isoquinolin-1-amine;
(4-((1-((6-chloropyridin-3-yl)amino)isoquinolin-6-yl)oxy)piperidin-1-yl)(cyclopropyl)methanone;
(S)-1-(3-((1-((6-chloropyridin-3-yl)amino)isoquinolin-6-yl)oxy)piperidin-1-yl)ethan-1-one;
(R)-1-(3-((1-((6-chloropyridin-3-yl)amino)isoquinolin-6-yl)oxy)piperidin-1-yl)ethan-1-one;
3-(((1-((6-chloropyridin-3-yl)amino)isoquinolin-6-yl)oxy)methyl)tetrahydrofuran-3-carbonitrile;
(R)-3-(((1-((6-chloropyridin-3-yl)amino)isoquinolin-6-yl)oxy)methyl)tetrahydrofuran-3-carbonitrile;
(S)-3-(((1-((6-chloropyridin-3-yl)amino)isoquinolin-6-yl)oxy)methyl)tetrahydrofuran-3-carbonitrile;
N-(6-chloropyridin-3-yl)-6-((3-fluoro-1-methylazetidin-3-yl)methoxy)isoquinolin-1-amine;
(R)—N-(6-chloropyridin-3-yl)-6-(1-methoxypropyl)isoquinolin-1-amine;
(S)—N-(6-chloropyridin-3-yl)-6-(1-methoxypropyl)isoquinolin-1-amine;
(R)—N-(6-chloropyridin-3-yl)-6-(1-methoxyethyl)isoquinolin-1-amine;
(S)—N-(6-chloropyridin-3-yl)-6-(1-methoxyethyl)isoquinolin-1-amine;
2-(3-(((1-((6-chloropyridin-3-yl)amino)isoquinolin-6-yl)oxy)methyl)oxetan-3-yl)acetonitrile;
(S)-6-((2-oxaspiro[3.4]octan-6-yl)oxy)-N-(6-chloropyridin-3-yl)isoquinolin-1-amine;
N-(6-chloropyridin-3-yl)-6-((2,2-dimethylbut-3-yn-1-yl)oxy)isoquinolin-1-amine;
6-([1,1′-bi(cyclopropan)]-1-ylmethoxy)-N-(6-chloropyridin-3-yl)isoquinolin-1-amine;
(R)-6-((2-oxaspiro[3.4]octan-6-yl)oxy)-N-(6-chloropyridin-3-yl)isoquinolin-1-amine;
(R)—N-(6-chloropyridin-3-yl)-6-(1-(1-fluorocyclopropyl)ethoxy)isoquinolin-1-amine;
(S)—N-(6-chloropyridin-3-yl)-6-(1-(1-fluorocyclopropyl)ethoxy)isoquinolin-1-amine;
N-(6-chloropyridin-3-yl)-6-((1-fluorocyclobutyl)methoxy)isoquinolin-1-amine;
cis-2-((1-((6-chloropyridin-3-yl)amino)isoquinolin-6-yl)oxy)cyclohexan-1-ol;
trans-2-((1-((6-chloropyridin-3-yl)amino)isoquinolin-6-yl)oxy)cyclohexan-1-ol;
(1 S,3R)-3-((1-((6-chloropyridin-3-yl)amino)isoquinolin-6-yl)oxy)-1-methylcyclohexan-1-ol;
(1R,3S)-3-((1-((6-chloropyridin-3-yl)amino)isoquinolin-6-yl)oxy)-1-methylcyclohexan-1-ol;
N-(6-chloropyridin-3-yl)-6-((1-methyl-1H-pyrazol-3-yl)methoxy)isoquinolin-1-amine;
N-(6-chloropyridin-3-yl)-6-((1-ethynylcyclopropyl)methoxy)isoquinolin-1-amine;
N-(6-chloropyridin-3-yl)-6-((3-isopropyloxetan-3-yl)methoxy)isoquinolin-1-amine;
6-((1-((6-chloropyridin-3-yl)amino)isoquinolin-6-yl)oxy)spiro[3.3]heptan-2-ol;
6-((1,4-dioxan-2-yl)methoxy)-N-(6-chloropyridin-3-yl)isoquinolin-1-amine;
3-((1-((6-chloropyridin-3-yl)amino)isoquinolin-6-yl)oxy)-2,2-difluoropropan-1-ol;
N-(6-chloropyridin-3-yl)-6-((5-methylisoxazol-4-yl)methoxy)isoquinolin-1-amine;
N-(6-chloropyridin-3-yl)-6-(isoxazol-5-ylmethoxy)isoquinolin-1-amine;
N-(6-chloropyridin-3-yl)-6-((2-(pyridin-3-ylmethyl)oxazol-5-yl)methoxy)isoquinolin-1-amine;
(R)—N-(6-chloropyridin-3-yl)-6-(2-(1-methyl-1H-pyrazol-4-yl)propoxy)isoquinolin-1-amine;
(S)—N-(6-chloropyridin-3-yl)-6-(2-(1-methyl-1H-pyrazol-4-yl)propoxy)isoquinolin-1-amine;
N-(6-chloropyridin-3-yl)-6-((2,2-dimethylpent-3-yn-1-yl)oxy)isoquinolin-1-amine;
2-chloro-N³-methyl-N⁵-(6-((3-methyloxetan-3-yl)methoxy)isoquinolin-1-yl)pyridine-3,5-diamine;
2-chloro-N⁵-(6-((3-methyloxetan-3-yl)methoxy)isoquinolin-1-yl)pyridine-3,5-diamine;
N-(6-chloropyridin-3-yl)-6-((1,5-dimethyl-1H-pyrazol-4-yl)methoxy)isoquinolin-1-amine;
N-(6-chloropyridin-3-yl)-6-((1,3-dimethyl-1H-pyrazol-4-yl)methoxy)isoquinolin-1-amine;
N-(6-chloropyridin-3-yl)-6-((3-methoxy-1-methyl-1H-pyrazol-4-yl)methoxy)isoquinolin-1-amine;
N-((1s,4s)-4-((1-((6-chloropyridin-3-yl)amino)isoquinolin-6-yl)oxy)cyclohexyl)-4-methylthiazole-5-carboxamide;
6-((5-(1H-1,2,4-triazol-1-yl)pentyl)oxy)-N-(6-chloropyridin-3-yl)isoquinolin-1-amine;
N-(6-chloropyridin-3-yl)-6-(1-(1-methyl-1H-pyrazol-4-yl)ethoxy)isoquinolin-1-amine;
(R)—N-(6-chloropyridin-3-yl)-6-(1-(1-methyl-1H-pyrazol-4-yl)ethoxy)isoquinolin-1-amine;
(S)—N-(6-chloropyridin-3-yl)-6-(1-(1-methyl-1H-pyrazol-4-yl)ethoxy)isoquinolin-1-amine;
N-(6-chloropyridin-3-yl)-6-(1-(1,3-dimethyl-1H-pyrazol-4-yl)ethoxy)isoquinolin-1-amine;
(R)—N-(6-chloropyridin-3-yl)-6-(1-(1,3-dimethyl-1H-pyrazol-4-yl)ethoxy)isoquinolin-1-amine;
(S)—N-(6-chloropyridin-3-yl)-6-(1-(1,3-dimethyl-1H-pyrazol-4-yl)ethoxy)isoquinolin-1-amine;
6-(2-amino-2,3-dimethylbutoxy)-N-(6-chloropyridin-3-yl)isoquinolin-1-amine;
N-(6-chloropyridin-3-yl)-6-((1-methoxycyclopropyl)methoxy)isoquinolin-1-amine;
N-(6-chloropyridin-3-yl)-6-((1-methylcyclopropyl)methoxy)isoquinolin-1-amine;
N-(6-chloropyridin-3-yl)-6-(2-cyclopropylethoxy)isoquinolin-1-amine;
N-(6-chloropyridin-3-yl)-6-((1-fluorocyclopropyl)methoxy)isoquinolin-1-amine;
N-(6-chloropyridin-3-yl)-6-(pyrimidin-5-ylmethoxy)isoquinolin-1-amine;
N-(6-chloropyridin-3-yl)-6-(2-(pyridin-2-yl)ethoxy)isoquinolin-1-amine;
N-(6-chloropyridin-3-yl)-6-(2-(trifluoromethoxy)ethoxy)isoquinolin-1-amine;
6-(3-(1H-imidazol-1-yl)propoxy)-N-(6-chloropyridin-3-yl)isoquinolin-1-amine;
N-(6-chloropyridin-3-yl)-6-((2-methoxypyrimidin-5-yl)methoxy)isoquinolin-1-amine;
N-(5-methoxy-6-methylpyridin-3-yl)-6-((3-methyloxetan-3-yl)methoxy)isoquinolin-1-amine;
N-(6-chloropyridin-3-yl)-6-((1-ethyl-1H-pyrazol-3-yl)methoxy)isoquinolin-1-amine;
6-((1-fluorocyclopropyl)methoxy)-N-(6-methylpyridin-3-yl)isoquinolin-1-amine;
N-(6-chloropyridin-3-yl)-6-(3-(methylsulfonyl)propoxy)isoquinolin-1-amine;
N-(6-chloropyridin-3-yl)-6-(pyridin-4-ylmethoxy)isoquinolin-1-amine;
N-(6-chloropyridin-3-yl)-6-(pyrimidin-2-ylmethoxy)isoquinolin-1-amine;
N-(6-chloropyridin-3-yl)-6-((6-methylpyridin-3-yl)methoxy)isoquinolin-1-amine;
N-(6-chloropyridin-3-yl)-6-(pyridin-3-ylmethoxy)isoquinolin-1-amine;
N-(6-chloropyridin-3-yl)-6-((1-methyl-1H-pyrazol-4-yl)methoxy)isoquinolin-1-amine;
N-(6-chloropyridin-3-yl)-6-((tetrahydro-2H-pyran-4-yl)oxy)isoquinolin-1-amine;
N-(6-chloropyridin-3-yl)-6-((1-methyl-1H-imidazol-2-yl)methoxy)isoquinolin-1-amine;
N-(6-methylpyridin-3-yl)-6-(pyridin-4-ylmethoxy)isoquinolin-1-amine;
N-(6-chloropyridin-3-yl)-6-(2-morpholinoethoxy)isoquinolin-1-amine;
N-(6-chloropyridin-3-yl)-6-((1,4-dimethyl-1H-pyrazol-3-yl)methoxy)isoquinolin-1-amine;
N-(6-chloropyridin-3-yl)-6-((4,4-dimethyloxetan-2-yl)methoxy)isoquinolin-1-amine;
N-(6-chloropyridin-3-yl)-6-((3-methyloxetan-3-yl)methoxy)isoquinolin-1-amine;
N-(6-chloropyridin-3-yl)-6-(oxazol-2-ylmethoxy)isoquinolin-1-amine;
N-(6-chloropyridin-3-yl)-6-(thiazol-2-ylmethoxy)isoquinolin-1-amine;
N-(6-methylpyridin-3-yl)-6-(oxetan-3-ylmethoxy)isoquinolin-1-amine;
N-(6-chloropyridin-3-yl)-6-(isoxazol-3-ylmethoxy)isoquinolin-1-amine;
N-(6-chloropyridin-3-yl)-6-((tetrahydro-2H-pyran-3-yl)oxy)isoquinolin-1-amine;
N-(6-chloropyridin-3-yl)-6-((tetrahydrofuran-2-yl)methoxy)isoquinolin-1-amine;
N-(6-chloropyridin-3-yl)-6-((3-methoxyoxetan-3-yl)methoxy)isoquinolin-1-amine;
N-(6-chloropyridin-3-yl)-6-(oxetan-3-ylmethoxy)isoquinolin-1-amine;
(R)-3-((1-((6-chloropyridin-3-yl)amino)isoquinolin-6-yl)oxy)-1,1,1-trifluoro-2-methylpropan-2-ol;
(S)-3-((1-((6-chloropyridin-3-yl)amino)isoquinolin-6-yl)oxy)-1,1,1-trifluoro-2-methylpropan-2-ol;
6-(2-amino-3,3,3-trifluoropropoxy)-N-(6-chloropyridin-3-yl)isoquinolin-1-amine;
(R)—N-(6-chloropyridin-3-yl)-6-((4,4-difluoropyrrolidin-2-yl)methoxy)isoquinolin-1-amine;
N¹-(6-chloropyridin-3-yl)-N⁶-(cyclopropylmethyl)-5-fluoroisoquinoline-1,6-diamine;
1-(((1-((6-chloropyridin-3-yl)amino)isoquinolin-6-yl)oxy)methyl)-3,3-difluorocyclobutane-1-carbonitrile;
N-(6-chloropyridin-3-yl)-6-(2-(2-methoxyethoxy)ethoxy)isoquinolin-1-amine;
(S)—N-(6-chloropyridin-3-yl)-6-(1-(pyridin-4-yl)ethoxy)isoquinolin-1-amine;
(1-(((1-((6-chloropyridin-3-yl)amino)isoquinolin-6-yl)oxy)methyl)cyclopropyl)methanol;
N-(6-chloropyridin-3-yl)-6-((4-fluorotetrahydro-2H-pyran-4-yl)methoxy)isoquinolin-1-amine;
3-((1-((6-chloropyridin-3-yl)amino)isoquinolin-6-yl)oxy)-1,1,1-trifluoropropan-2-ol;
N′-(6-chloropyridin-3-yl)-N⁶-((1-methyl-1H-pyrazol-4-yl)methyl)isoquinoline-1,6-diamine;
N′-(6-chloropyridin-3-yl)-N⁶-((3-methyloxetan-3-yl)methyl)isoquinoline-1,6-diamine;
(E)-3-(1-((6-chloropyridin-3-yl)amino)isoquinolin-6-yl)-N,N-dimethylacrylamide;
N-(6-chloropyridin-3-yl)-7-fluoro-6-((1-fluorocyclopropyl)methoxy)isoquinolin-1-amine;
6-(((1H-pyrazol-4-yl)amino)methyl)-N-(6-chloropyridin-3-yl)isoquinolin-1-amine;
N-(6-chloropyridin-3-yl)-6-(((1-methyl-1H-pyrazol-4-yl)oxy)methyl)isoquinolin-1-amine;
N-(6-chloropyridin-3-yl)-6-((methyl(1H-pyrazol-4-yl)amino)methyl)isoquinolin-1-amine;
6-(3-(1H-pyrazol-4-yl)propoxy)-N-(6-chloropyridin-3-yl)isoquinolin-1-amine;
(R)-3-((1-((6-chloropyridin-3-yl)amino)isoquinolin-6-yl)oxy)tetrahydrothiophene 1,1-dioxide;
(S)-3-((1-((6-chloropyridin-3-yl)amino)isoquinolin-6-yl)oxy)tetrahydrothiophene 1,1-dioxide;
N-(6-chloropyridin-3-yl)-6-((tetrahydrofuran-3-yl)oxy)isoquinolin-1-amine;
trans-4-((1-((6-chloropyridin-3-yl)amino)isoquinolin-6-yl)oxy)-1-methylcyclohexan-1-ol;
cis-4-((1-((6-chloropyridin-3-yl)amino)isoquinolin-6-yl)oxy)-1-methylcyclohexan-1-ol;
N-(6-chloropyridin-3-yl)-6-(pyridin-3-yloxy)isoquinolin-1-amine;
6-((1H-pyrazol-4-yl)oxy)-N-(6-chloropyridin-3-yl)isoquinolin-1-amine;
((1-((6-chloropyridin-3-yl)amino)isoquinolin-6-yl)imino)(cyclopropyl)(methyl)-λ⁶-sulfanone;
((1-((6-chloropyridin-3-yl)amino)isoquinolin-6-yl)imino)(cyclopropylmethyl)(methyl)-λ⁶-sulfanone;
((1-((6-chloropyridin-3-yl)amino)isoquinolin-6-yl)imino)(methyl)(tetrahydro-2H-pyran-4-yl)-λ⁶-sulfanone;
((1-((6-chloropyridin-3-yl)amino)isoquinolin-6-yl)imino)(cyclopropyl)(2-methoxyethyl)-λ⁶-sulfanone;
((1-((6-chloropyridin-3-yl)amino)isoquinolin-6-yl)imino)(methyl)(oxetan-3-ylmethyl)-λ⁶-sulfanone
(R)-((1-((6-chloropyridin-3-yl)amino)isoquinolin-6-yl)imino)(cyclopropyl)(methyl)-λ⁶-sulfanone;
(S)-((1-((6-chloropyridin-3-yl)amino)isoquinolin-6-yl)imino)(cyclopropyl)(methyl)-λ⁶-sulfanone;
((1-((6-chloropyridin-3-yl)amino)isoquinolin-6-yl)imino)(methyl)(oxetan-3-yl)-λ⁶-sulfanone;
(S)-((1-((6-chloropyridin-3-yl)amino)isoquinolin-6-yl)imino)(methyl)(oxetan-3-yl)-λ⁶-sulfanone);
(R)-((1-((6-chloropyridin-3-yl)amino)isoquinolin-6-yl)imino)(methyl)(oxetan-3-yl)-λ⁶-sulfanone);
N-(6-chloropyridin-3-yl)-6-((1-(difluoromethyl)-1H-pyrazol-4-yl)methoxy)isoquinolin-1-amine;
(R)-6-((1-benzylpyrrolidin-2-yl)methoxy)-N-(6-chloropyridin-3-yl)isoquinolin-1-amine;
N-(6-chloropyridin-3-yl)-6-((5,6-dihydro-4H-pyrrolo[1,2-b]pyrazol-3-yl)methoxy)isoquinolin-1-amine;
6-((1-benzylpyrrolidin-3-yl)oxy)-N-(6-chloropyridin-3-yl)isoquinolin-1-amine;
(R)-6-((1-benzylpyrrolidin-3-yl)methoxy)-N-(6-chloropyridin-3-yl)isoquinolin-1-amine;
(S)-6-((1-benzylpyrrolidin-3-yl)methoxy)-N-(6-chloropyridin-3-yl)isoquinolin-1-amine;
N-(6-chloropyridin-3-yl)-6-((4,5,6,7-tetrahydropyrazolo[1,5-a]pyridin-3-yl)methoxy)isoquinolin-1-amine;
N-(6-chloropyridin-3-yl)-6-((1-(methylsulfonyl)cyclopropyl)methoxy)isoquinolin-1-amine;
N-(6-chloropyridin-3-yl)-6-((1-(methylsulfonyl)cyclobutyl)methoxy)isoquinolin-1-amine;
N-(6-chloropyridin-3-yl)-6-((1-methoxycyclobutyl)methoxy)isoquinolin-1-amine;
N-(6-chloropyridin-3-yl)-6-((1-cyclopropyl-1H-pyrazol-4-yl)methoxy)isoquinolin-1-amine;
6-((2-oxaspiro[3.3]heptan-5-yl)oxy)-N-(6-chloropyridin-3-yl)isoquinolin-1-amine;
6-((5-chloro-1-methyl-1H-pyrazol-4-yl)methoxy)-N-(6-chloropyridin-3-yl)isoquinolin-1-amine;
6-((3-chloro-1-methyl-1H-pyrazol-4-yl)methoxy)-N-(6-chloropyridin-3-yl)isoquinolin-1-amine;
3-(((1-((6-chloropyridin-3-yl)amino)isoquinolin-6-yl)oxy)methyl)-3-methylthietane 1,1-dioxide;
1-(((1-((6-chloropyridin-3-yl)amino)isoquinolin-6-yl)oxy)methyl)cyclobutane-1-sulfonamide;
6-((1-benzyl-1H-pyrazol-4-yl)methoxy)-N-(6-chloropyridin-3-yl)isoquinolin-1-amine;
N-(6-chloropyridin-3-yl)-6-((1-(1-methyl-1H-pyrazol-4-yl)propan-2-yl)oxy)isoquinolin-1-amine;
(R)—N-(6-chloropyridin-3-yl)-6-((tetrahydrofuran-3-yl)oxy)isoquinolin-1-amine;
(S)—N-(6-chloropyridin-3-yl)-6-((tetrahydrofuran-3-yl)oxy)isoquinolin-1-amine;
N-(6-chloropyridin-3-yl)-6-((2,2-dimethyl-1,3-dioxolan-4-yl)methoxy)isoquinolin-1-amine;
3-(((1-((6-chloropyridin-3-yl)amino)isoquinolin-6-yl)oxy)methyl)thietane 1,1-dioxide;
N-(6-chloropyridin-3-yl)-6-(isothiazol-4-ylmethoxy)isoquinolin-1-amine;
(R)—N-(6-chloropyridin-3-yl)-6-((2-methyltetrahydrofuran-2-yl)methoxy)isoquinolin-1-amine;
(S)—N-(6-chloropyridin-3-yl)-6-((2-methyltetrahydrofuran-2-yl)methoxy)isoquinolin-1-amine;
6-(((1H-pyrazol-4-yl)oxy)methyl)-N-(6-chloropyridin-3-yl)isoquinolin-1-amine;
1-(4-((1-((6-chloropyridin-3-yl)amino)isoquinolin-6-yl)oxy)piperidin-1-yl)ethan-1-one;
N-(6-chloropyridin-3-yl)-6-((3-(methoxymethyl)oxetan-3-yl)methoxy)isoquinolin-1-amine;
N-(6-chloropyridin-3-yl)-6-((1-methyl-1H-1,2,4-triazol-3-yl)methoxy)isoquinolin-1-amine;
1-(3-(((1-((6-chloropyridin-3-yl)amino)isoquinolin-6-yl)oxy)methyl)azetidin-1-yl)ethan-1-one;
N-(6-chloropyridin-3-yl)-6-((1-(2,2,2-trifluoroethyl)azetidin-3-yl)methoxy)isoquinolin-1-amine;
N-(6-chloropyridin-3-yl)-6-((1-methyl-1H-pyrazol-4-yl)oxy)isoquinolin-1-amine;
1-(4-((1-((6-chloropyridin-3-yl)amino)isoquinolin-6-yl)oxy)piperidin-1-yl)ethan-1-one;
N-(6-chloropyridin-3-yl)-6-((1-(oxetan-3-yl)piperidin-4-yl)oxy)isoquinolin-1-amine;
N-(6-chloropyridin-3-yl)-6-((1-(pyrimidin-2-ylmethyl)piperidin-4-yl)oxy)isoquinolin-1-amine;
N-(6-chloropyridin-3-yl)-6-((1-(2,2-difluoroethyl)piperidin-4-yl)oxy)isoquinolin-1-amine;
N-(cis-4-((1-((6-chloropyridin-3-yl)amino)isoquinolin-6-yl)oxy)cyclohexyl)acetamide;
N-(trans-4-((1-((6-chloropyridin-3-yl)amino)isoquinolin-6-yl)oxy)cyclohexyl)acetamide;
1-(4-(((1-((6-chloropyridin-3-yl)amino)isoquinolin-6-yl)oxy)methyl)piperidin-1-yl)ethan-1-one;
N-(6-chloropyridin-3-yl)-6-(2-((2S,6R)-2,6-dimethylmorpholino)ethoxy)isoquinolin-1-amine;
methyl 4-(2-((1-((6-chloropyridin-3-yl)amino)isoquinolin-6-yl)oxy)ethyl)morpholine-3-carboxylate;
(S)—N-(6-chloropyridin-3-yl)-6-(2-(3-methylmorpholino)ethoxy)isoquinolin-1-amine;
(R)—N-(6-chloropyridin-3-yl)-6-(2-(3-methylmorpholino)ethoxy)isoquinolin-1-amine;
N-(6-chloropyridin-3-yl)-6-(2-(2-methylmorpholino)ethoxy)isoquinolin-1-amine;
2-methyl-5-((6-((1-methyl-1H-pyrazol-4-yl)methoxy)isoquinolin-1-yl)amino)pyridin-3-ol;
N-(6-chloropyridin-3-yl)-6-((1-(2,2,2-trifluoroethyl)azetidin-3-yl)oxy)isoquinolin-1-amine;
N-(6-chloropyridin-3-yl)-6-((1-(difluoromethyl)cyclopropyl)methoxy)isoquinolin-1-amine;
N-(6-chloropyridin-3-yl)-6-((3-(difluoromethyl)oxetan-3-yl)methoxy)isoquinolin-1-amine;
N-(6-chloropyridin-3-yl)-6-((3-ethyloxetan-3-yl)methoxy)isoquinolin-1-amine;
(3-(((1-((6-chloropyridin-3-yl)amino)isoquinolin-6-yl)oxy)methyl)oxetan-3-yl)methanol;
1-(((1-((6-chloropyridin-3-yl)amino)isoquinolin-6-yl)oxy)methyl)-2,2-dimethylcyclopropane-1-carbonitrile;
3-((1-((6-chloropyridin-3-yl)amino)isoquinolin-6-yl)oxy)adamantan-1-ol;
N-(6-chloropyridin-3-yl)-6-(spiro[2.3]hexan-1-ylmethoxy)isoquinolin-1-amine;
6-(((1-((6-chloropyridin-3-yl)amino)isoquinolin-6-yl)oxy)methyl)-2-oxaspiro[3.3]heptane-6-carbonitrile;
6-(1-(1H-pyrazol-4-yl)ethoxy)-N-(6-chloropyridin-3-yl)isoquinolin-1-amine;
6-(1-(1H-pyrazol-4-yl)ethoxy)-N-(6-chloropyridin-3-yl)isoquinolin-1-amine;
N-(6-chloropyridin-3-yl)-6-((1-(2-methoxyethyl)-1H-pyrazol-4-yl)methoxy)isoquinolin-1-amine;
6-((3-chloro-1H-pyrazol-4-yl)methoxy)-N-(6-chloropyridin-3-yl)isoquinolin-1-amine;
N-(6-chloropyridin-3-yl)-6-((3,5-dimethyl-1H-pyrazol-4-yl)methoxy)isoquinolin-1-amine;
N-(6-chloropyridin-3-yl)-6-(2-(3-methyloxetan-3-yl)ethyl)isoquinolin-1-amine;
(1 S,3S)-3-((1-((6-chloropyridin-3-yl)amino)isoquinolin-6-yl)oxy)cyclohexane-1-carbonitrile;
(1R,3S)-3-((1-((6-chloropyridin-3-yl)amino)isoquinolin-6-yl)oxy)cyclohexane-1-carbonitrile;
(1R,3R)-3-((1-((6-chloropyridin-3-yl)amino)isoquinolin-6-yl)oxy)cyclohexane-1-carbonitrile;
(1 S,3R)-3-((1-((6-chloropyridin-3-yl)amino)isoquinolin-6-yl)oxy)cyclohexane-1-carbonitrile;
(1R,3S)-3-((1-((6-chloropyridin-3-yl)amino)isoquinolin-6-yl)oxy)cyclohexan-1-ol;
(1 S,3R)-3-((1-((6-chloropyridin-3-yl)amino)isoquinolin-6-yl)oxy)cyclohexan-1-ol;
(1R,4R)-4-((1-((6-chloropyridin-3-yl)amino)isoquinolin-6-yl)oxy)cyclohexan-1-ol;
trans-3-((1-((6-chloropyridin-3-yl)amino)isoquinolin-6-yl)oxy)cyclohexan-1-ol;
N-(6-chloropyridin-3-yl)-6-((1-(pyridin-4-ylmethoxy)cyclopropyl)methoxy)isoquinolin-1-amine;
N-(6-chloropyridin-3-yl)-6-((3-fluorotetrahydrofuran-3-yl)methoxy)isoquinolin-1-amine;
(R)—N-(6-chloropyridin-3-yl)-6-((3-fluorotetrahydrofuran-3-yl)methoxy)isoquinolin-1-amine;
(S)—N-(6-chloropyridin-3-yl)-6-((3-fluorotetrahydrofuran-3-yl)methoxy)isoquinolin-1-amine;
N-(6-chloropyridin-3-yl)-6-(isoxazol-4-ylmethoxy)isoquinolin-1-amine;
2-chloro-5-((6-((1-methyl-1H-pyrazol-4-yl)methoxy)isoquinolin-1-yl)amino)pyridin-3-ol;
N-(6-chloropyridin-3-yl)-6-((5-cyclopropyl-1H-pyrazol-4-yl)methoxy)isoquinolin-1-amine;
N-(6-chloropyridin-3-yl)-6-((1-(2,2-difluoroethyl)-1H-pyrazol-4-yl)methoxy)isoquinolin-1-amine;
N-(6-chloropyridin-3-yl)-6-((1-methyl-1H-pyrazol-4-yl)methoxy-d₂)isoquinolin-1-amine;
N-(6-chloropyridin-3-yl)-6-(2-(pyrimidin-2-yl)ethoxy)isoquinolin-1-amine;
6-((1H-pyrazol-3-yl)methoxy)-N-(6-chloropyridin-3-yl)isoquinolin-1-amine;
N-(6-chloropyridin-3-yl)-6-((5-methyl-1H-pyrazol-3-yl)methoxy)isoquinolin-1-amine;
N-(6-chloropyridin-3-yl)-6-((3-methyl-1H-pyrazol-4-yl)methoxy)isoquinolin-1-amine;
3-(((1-((6-chloropyridin-3-yl)amino)isoquinolin-6-yl)oxy)methyl)oxetan-3-ol;
N-(6-chloropyridin-3-yl)-6-((4-fluoro-1-methyl-1H-pyrazol-3-yl)methoxy)isoquinolin-1-amine;
N-(6-chloropyridin-3-yl)-3-methylisoquinolin-1-amine;
2-(4-(((1-((6-chloropyridin-3-yl)amino)isoquinolin-6-yl)oxy)methyl)-1H-pyrazol-1-yl)acetonitrile;
N-(1-((6-chloropyridin-3-yl)amino)isoquinolin-6-yl)-1-(hydroxymethyl)cyclopropane-1-carboxamide;
N-(6-chloropyridin-3-yl)-6-((2,2-difluorocyclopropyl)methoxy)isoquinolin-1-amine;
(S)—N-(6-chloropyridin-3-yl)-6-((5,5-dimethyltetrahydrofuran-2-yl)methoxy)isoquinolin-1-amine;
N-(6-chloropyridin-3-yl)-6-((2-methyltetrahydrofuran-2-yl)methoxy)isoquinolin-1-amine;
N-(6-chloropyridin-3-yl)-6-((3,3-difluorocyclobutyl)methoxy)isoquinolin-1-amine;
N-(6-chloropyridin-3-yl)-6-((1r,3r)-3-fluorocyclobutoxy)isoquinolin-1-amine;
N-(6-chloropyridin-3-yl)-6-(2,2,3,3-tetrafluoropropoxy)isoquinolin-1-amine;
N-(6-chloropyridin-3-yl)-4,6-dimethoxyisoquinolin-1-amine;
N-(6-chloropyridin-3-yl)-6-cyclobutoxyisoquinolin-1-amine;
N-(6-chloropyridin-3-yl)-6-(3,3-difluorocyclobutoxy)isoquinolin-1-amine;
N-(6-chloropyridin-3-yl)-6-(((1S,2R)-2-fluorocyclopropyl)methoxy)isoquinolin-1-amine;
N-(6-chloropyridin-3-yl)-6-(((1S,2S)-2-fluorocyclopropyl)methoxy)isoquinolin-1-amine;
N-(6-chloropyridin-3-yl)-6-((tetrahydrofuran-3-yl)methoxy)isoquinolin-1-amine;
(R)—N-(6-chloropyridin-3-yl)-6-((5,5-dimethyltetrahydrofuran-2-yl)methoxy)isoquinolin-1-amine;
N-(6-chloropyridin-3-yl)-6-(2,2-difluoroethoxy)isoquinolin-1-amine;
1-((6-chloropyridin-3-yl)amino)-N-(2-methoxyethyl)isoquinoline-6-carboxamide;
1-((6-chloropyridin-3-yl)amino)-N-(cyclopropylmethyl)isoquinoline-6-carboxamide;
N-(6-chloropyridin-3-yl)-6-(pyrrolidin-1-yl)isoquinolin-1-amine;
N-(6-chloropyridin-3-yl)-6-(1-methyl-1H-pyrazol-5-yl)isoquinolin-1-amine;
N-(6-chloropyridin-3-yl)-6-(pyrimidin-5-yl)isoquinolin-1-amine;
N-(6-chloropyridin-3-yl)-6-(1H-pyrazol-3-yl)isoquinolin-1-amine;
6-((2H-1,2,3-triazol-2-yl)methyl)-N-(6-chloropyridin-3-yl)isoquinolin-1-amine;
N-(6-chloropyridin-3-yl)-6-(pyridin-2-ylmethyl)isoquinolin-1-amine; and
6-((3-methyl-1H-pyrazol-4-yl)methoxy)-N-(6-methylpyridin-3-yl)isoquinolin-1-amine, as an individual stereoisomer, enantiomer or tautomer thereof or a mixture thereof;
or a pharmaceutically acceptable salt, solvate or prodrug thereof.

Another embodiment of a compound of Formula (I), as described above in the Brief Summary, is a compound of Formula (I) wherein Y is ═N— having the following formula (Ia2):

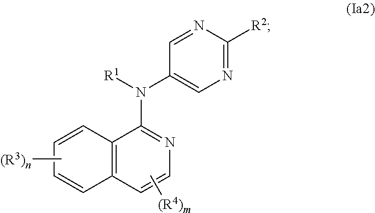

wherein m, n, R¹, R², R³ and R⁴ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1, 2 or 3;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸; each R⁴ is independently alkyl or —R⁹—OR⁶;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1, 2 or 3;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) selected from the following:
N-(2-chloropyrimidin-5-yl)-6-(cyclopropylmethoxy)isoquinolin-1-amine;
N-(2-chloropyrimidin-5-yl)-6-fluoroisoquinolin-1-amine;
6-chloro-N-(2-chloropyrimidin-5-yl)isoquinolin-1-amine;
6-(cyclopropylmethoxy)-N-(2-methylpyrimidin-5-yl)isoquinolin-1-amine;
N-(2-methylpyrimidin-5-yl)-6-((tetrahydrofuran-3-yl)methoxy)isoquinolin-1-amine;
6-(2,2-difluoroethoxy)-N-(2-methylpyrimidin-5-yl)isoquinolin-1-amine;
6-((5,5-dimethyltetrahydrofuran-2-yl)methoxy)-N-(2-methylpyrimidin-5-yl)isoquinolin-1-amine;
6-(2-methoxyethoxy)-N-(2-methylpyrimidin-5-yl)isoquinolin-1-amine;
6-(2-cyclopropoxyethoxy)-N-(2-methylpyrimidin-5-yl)isoquinolin-1-amine;
3-(((1-((2-chloropyrimidin-5-yl)amino)isoquinolin-6-yl)oxy)methyl)bicyclo[1.1.1]pentane-1-carbonitrile;
N-(2-chloropyrimidin-5-yl)-6-((3-isopropyloxetan-3-yl)methoxy)isoquinolin-1-amine;
N-(2-ethylpyrimidin-5-yl)-6-((1-fluorocyclopropyl)methoxy)isoquinolin-1-amine;
6-((1-fluorocyclopropyl)methoxy)-N-(2-methoxypyrimidin-5-yl)isoquinolin-1-amine;
6-((1-fluorocyclopropyl)methoxy)-N-(pyrimidin-5-yl)isoquinolin-1-amine;
N-(2-chloropyrimidin-5-yl)-6-((3-fluorooxetan-3-yl)methoxy)isoquinolin-1-amine;
6-((3-fluorooxetan-3-yl)methoxy)-N-(2-methylpyrimidin-5-yl)isoquinolin-1-amine;
6-((3-fluoroazetidin-3-yl)methoxy)-N-(2-methylpyrimidin-5-yl)isoquinolin-1-amine;
1-((1-((2-chloropyrimidin-5-yl)amino)isoquinolin-6-yl)oxy)cyclopropane-1-carbonitrile;
1-((1-((2-chloropyrimidin-5-yl)amino)isoquinolin-6-yl)oxy)cyclopropane-1-carboxamide;
(1s,3s)-3-(((1-((2-chloropyrimidin-5-yl)amino)isoquinolin-6-yl)oxy)methyl)-3-fluorocyclobutane-1-carbonitrile;
N-(2-chloropyrimidin-5-yl)-6-((1-fluorocyclopropyl)methoxy)isoquinolin-1-amine;
1-(((1-((2-methylpyrimidin-5-yl)amino)isoquinolin-6-yl)oxy)methyl)cyclopropane-1-carbonitrile;
1-(((1-((2-methylpyrimidin-5-yl)amino)isoquinolin-6-yl)oxy)methyl)cyclopropane-1-carbonitrile;
(5-((6-((1-fluorocyclopropyl)methoxy)isoquinolin-1-yl)amino)pyrimidin-2-yl)methanol;
N-(2-chloropyrimidin-5-yl)-6-((1-fluorocyclopropyl)methoxy)-N-methylisoquinolin-1-amine;
rac-(1R,3S)-3-((1-((2-chloropyrimidin-5-yl)amino)isoquinolin-6-yl)oxy)-1-(trifluoromethyl)cyclohexan-1-ol;
3-((1-((2-chloropyrimidin-5-yl)amino)isoquinolin-6-yl)oxy)-2-cyclopropyl-2-fluoropropanenitrile;
2-(1-(((1-((2-chloropyrimidin-5-yl)amino)isoquinolin-6-yl)oxy)methyl)cyclopropyl)acetonitrile;
6-(1-(1-methyl-1H-pyrazol-4-yl)ethoxy)-N-(2-methylpyrimidin-5-yl)isoquinolin-1-amine;
6-((1-methoxycyclopropyl)methoxy)-N-(2-methylpyrimidin-5-yl)isoquinolin-1-amine;
6-((1-fluorocyclopropyl)methoxy)-N-(2-methylpyrimidin-5-yl)isoquinolin-1-amine;
N-(2-methylpyrimidin-5-yl)-6-(pyridin-4-ylmethoxy)isoquinolin-1-amine;
N-(2-chloropyrimidin-5-yl)-6-(pyridin-4-ylmethoxy)isoquinolin-1-amine;
N-(2-chloropyrimidin-5-yl)-6-((1-methyl-1H-pyrazol-3-yl)methoxy)isoquinolin-1-amine;
6-((1-methyl-1H-pyrazol-4-yl)methoxy)-N-(2-methylpyrimidin-5-yl)isoquinolin-1-amine;
6-((1-methyl-1H-pyrazol-3-yl)methoxy)-N-(2-methylpyrimidin-5-yl)isoquinolin-1-amine;
1-(((1-((2-chloropyrimidin-5-yl)amino)isoquinolin-6-yl)oxy)methyl)cyclopropane-1-carbonitrile;
3-(((1-((2-chloropyrimidin-5-yl)amino)isoquinolin-6-yl)oxy)methyl)oxetane-3-carbonitrile;
N-(2-chloropyrimidin-5-yl)-6-(oxetan-3-ylmethoxy)isoquinolin-1-amine;
6-((3-methyloxetan-3-yl)methoxy)-N-(2-methylpyrimidin-5-yl)isoquinolin-1-amine;
6-((3-methoxyoxetan-3-yl)methoxy)-N-(2-methylpyrimidin-5-yl)isoquinolin-1-amine;
N-(2-methylpyrimidin-5-yl)-6-(oxetan-3-ylmethoxy)isoquinolin-1-amine;
6-(isoxazol-3-ylmethoxy)-N-(2-methylpyrimidin-5-yl)isoquinolin-1-amine;
3-(((1-((2-methylpyrimidin-5-yl)amino)isoquinolin-6-yl)oxy)methyl)oxetane-3-carbonitrile;
6-(cyclopropylmethoxy)-5-fluoro-N-(2-methylpyrimidin-5-yl)isoquinolin-1-amine;
3,3-difluoro-1-(((1-((2-methylpyrimidin-5-yl)amino)isoquinolin-6-yl)oxy)methyl)cyclobutane-1-carbonitrile;
6-(isoxazol-4-ylmethoxy)-N-(2-methylpyrimidin-5-yl)isoquinolin-1-amine;
N-(2-chloropyrimidin-5-yl)-6-(isoxazol-4-ylmethoxy)isoquinolin-1-amine;
1-((2-chloropyrimidin-5-yl)amino)isoquinolin-6-ol;
cyclopropyl(methyl)((1-((2-methylpyrimidin-5-yl)amino)isoquinolin-6-yl)imino)-A6-sulfanone;
N-(2-methylpyrimidin-5-yl)-6-(pyrimidin-5-ylmethoxy)isoquinolin-1-amine;
(1R,3S)-3-((1-((2-methylpyrimidin-5-yl)amino)isoquinolin-6-yl)oxy)cyclohexan-1-ol;
(1S,3R)-3-((1-((2-methylpyrimidin-5-yl)amino)isoquinolin-6-yl)oxy)cyclohexan-1-ol;
cis-3-((1-((2-chloropyrimidin-5-yl)amino)isoquinolin-6-yl)oxy)cyclohexan-1-ol;
1-(((1-((2-chloropyrimidin-5-yl)amino)isoquinolin-6-yl)oxy)methyl)spiro[2.2]pentane-1-carbonitrile;
1-(((1-((2-chloropyrimidin-5-yl)amino)isoquinolin-6-yl)oxy)methyl)-2,2-dimethylcyclopropane-1-carbonitrile;
N-(2-chloropyrimidin-5-yl)-6-((1,5-dimethyl-1H-pyrazol-4-yl)methoxy)isoquinolin-1-amine;
6-((1,5-dimethyl-1H-pyrazol-4-yl)methoxy)-N-(2-methylpyrimidin-5-yl)isoquinolin-1-amine;
N-(2-chloropyrimidin-5-yl)-6-((3-(1,1-difluoroethyl)oxetan-3-yl)methoxy)isoquinolin-1-amine;
tert-butyl ((1S,3R)-3-((1-((2-chloropyrimidin-5-yl)amino)isoquinolin-6-yl)oxy)cyclohexyl)carbamate;
cis-3-((1-((2-methylpyrimidin-5-yl)amino)isoquinolin-6-yl)oxy)cyclohexan-1-ol;
(1R,3S)-3-((1-((2-chloropyrimidin-5-yl)amino)isoquinolin-6-yl)oxy)cyclohexan-1-ol;
(1S,3R)-3-((1-((2-chloropyrimidin-5-yl)amino)isoquinolin-6-yl)oxy)cyclohexan-1-ol;
cis-N-(2-chloropyrimidin-5-yl)-6-(((1S,3R)-3-methoxycyclohexyl)oxy)isoquinolin-1-amine;
1-(((1-((2-chloropyrimidin-5-yl)amino)isoquinolin-6-yl)oxy)methyl)cyclopropane-1-carboxamide;
cis-3-(((1-((2-chloropyrimidin-5-yl)amino)isoquinolin-6-yl)oxy)methyl)-1-iminohexahydro-1λ6-thiopyran 1-oxide;
trans-3-(((1-((2-chloropyrimidin-5-yl)amino)isoquinolin-6-yl)oxy)methyl)-1-iminohexahydro-1λ6-thiopyran 1-oxide;
1-(((1-((2-chloropyrimidin-5-yl)amino)isoquinolin-6-yl)oxy)methyl)cyclobutane-1-carbonitrile;
N-(2-methoxypyrimidin-5-yl)-6-((3-methyloxetan-3-yl)methoxy)isoquinolin-1-amine;
N-(2-methoxypyrimidin-5-yl)-6-((1-methyl-1H-pyrazol-4-yl)methoxy)isoquinolin-1-amine;
6-(cyclopropylmethoxy)-N-(2-methoxypyrimidin-5-yl)isoquinolin-1-amine;
1-(((1-((2-methoxypyrimidin-5-yl)amino)isoquinolin-6-yl)oxy)methyl)cyclopropane-1-carbonitrile
N-((1R,3S)-3-((1-((2-chloropyrimidin-5-yl)amino)isoquinolin-6-yl)oxy)cyclohexyl)-2,2,2-trifluoroacetamide;
N-((1R,3S)-3-((1-((2-chloropyrimidin-5-yl)amino)isoquinolin-6-yl)oxy)cyclohexyl)acetamide;
N-((1S,3R)-3-((1-((2-chloropyrimidin-5-yl)amino)isoquinolin-6-yl)oxy)cyclohexyl)-2,2,2-trifluoroacetamide;
N-((1S,3R)-3-((1-((2-chloropyrimidin-5-yl)amino)isoquinolin-6-yl)oxy)cyclohexyl)acetamide;
6-((1H-pyrazol-4-yl)methoxy)-N-(2-chloropyrimidin-5-yl)isoquinolin-1-amine;
(R)-6-((1,4-dioxan-2-yl)methoxy)-N-(2-chloropyrimidin-5-yl)isoquinolin-1-amine;
(S)-6-((1,4-dioxan-2-yl)methoxy)-N-(2-chloropyrimidin-5-yl)isoquinolin-1-amine;
N-(2-chloropyrimidin-5-yl)-6-((1-fluorocyclopropyl)methoxy-d2)isoquinolin-1-amine;
1-(1-((1-((2-chloropyrimidin-5-yl)amino)isoquinolin-6-yl)oxy)ethyl)cyclopropane-1-carbonitrile;
N-(2-methoxypyrimidin-5-yl)-6-((5-methyl-1H-pyrazol-4-yl)methoxy)isoquinolin-1-amine;
6-(3-(1H-pyrazol-4-yl)propoxy)-N-(2-chloropyrimidin-5-yl)isoquinolin-1-amine;
6-(2-(1H-pyrazol-4-yl)ethoxy)-N-(2-chloropyrimidin-5-yl)isoquinolin-1-amine;
6-((1H-pyrazol-4-yl)methoxy)-N-(2-methylpyrimidin-5-yl)isoquinolin-1-amine;
6-((3-methyl-1H-pyrazol-4-yl)methoxy)-N-(2-methylpyrimidin-5-yl)isoquinolin-1-amine; and
N-(2-chloropyrimidin-5-yl)-6-((5,5-dimethyltetrahydrofuran-2-yl)methoxy)isoquinolin-1-amine;
as an individual stereoisomer, enantiomer, or tautomer thereof or a mixture thereof;
or a pharmaceutically acceptable salt, solvate or prodrug thereof.

Another embodiment of a compound of Formula (I), as described above in the Brief Summary, is a compound of Formula (I) wherein [symbol] is a fused heteroaryl and m, n, Y, R¹, R², R³ and R⁴ are each as described above in the Brief Summary; as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein [symbol] is a fused heteroaryl selected from N-heteroaryl, O-heteroaryl, S-heteroaryl and S,N-heteroaryl and m, n, Y, R¹, R², R³ and R⁴ are each as described above in the Brief Summary; as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein [symbol] is N-heteroaryl having one of the following formula (Ib) or formula (Ic):

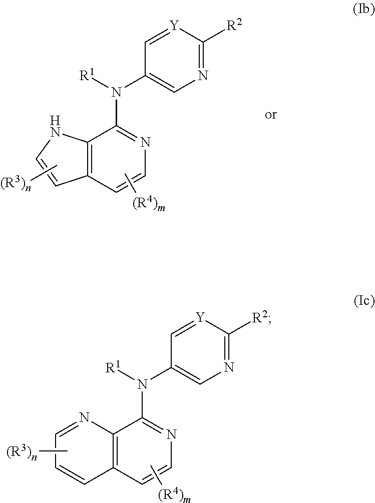

wherein n is 0, 1 or 2 and m, Y, R¹, R², R³ and R⁴ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein Y is ═C(R⁵)— having the formula (Ib1) or formula (Ic1):

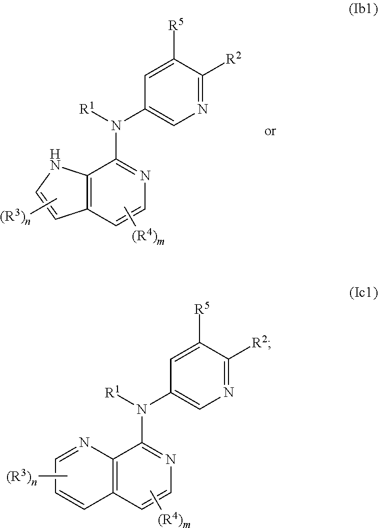

wherein n is 0, 1 or 2 and m, R¹, R², R³, R⁴ and R⁵ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl, —R⁹—OR⁶, heterocyclyl, or heterocyclylalkyl;
each R⁹ is independently a direct bond;
R¹⁰ is a straight or branched alkylene chain; and
R¹¹ is hydrogen, alkyl, alkoxyalkyl, or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) selected from the following:
N-(6-chloropyridin-3-yl)-1H-pyrrolo[2,3-c]pyridin-7-amine;
N-(6-chloropyridin-3-yl)-4-methoxy-1H-pyrrolo[2,3-c]pyridin-7-amine;
N-(6-chloropyridin-3-yl)-1,7-naphthyridin-8-amine;
N-(6-chloropyridin-3-yl)-4-methyl-1H-pyrrolo[2,3-c]pyridin-7-amine;
N⁸-(6-chloropyridin-3-yl)-N³-((1-methyl-1H-pyrazol-4-yl)methyl)-1,7-naphthyridine-3,8-diamine;
N-(6-chloropyridin-3-yl)-3-((1-methyl-1H-pyrazol-4-yl)methoxy)-1,7-naphthyridin-8-amine;
N-(6-chloropyridin-3-yl)-3-(cyclopropylmethoxy)-1,7-naphthyridin-8-amine;
3-(cyclopropylmethoxy)-N-(6-methylpyridin-3-yl)-1,7-naphthyridin-8-amine;
N-(6-chloropyridin-3-yl)-3-((3-methyloxetan-3-yl)methoxy)-1,7-naphthyridin-8-amine;
N-(6-chloropyridin-3-yl)-3-methoxy-1,7-naphthyridin-8-amine;
N⁷-(6-chloropyridin-3-yl)-N⁴-(2,2,2-trifluoroethyl)-1H-pyrrolo[2,3-c]pyridine-4,7-diamine; and
N⁴-benzyl-N-(6-chloropyridin-3-yl)-1H-pyrrolo[2,3-c]pyridine-4,7-diamine,
as an individual stereoisomer, enantiomer, or tautomer thereof or a mixture thereof;
or a pharmaceutically acceptable salt, solvate or prodrug thereof.

Another embodiment of a compound of Formula (I), as described above in the Brief Summary, is a compound of Formula (I) wherein Y is ═N— having one of the following formula (Ib2) or formula (Ic2):

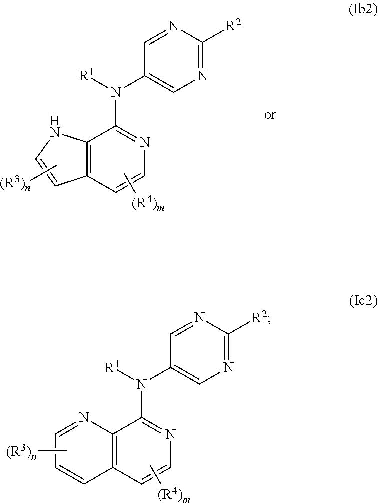

wherein n is 0, 1 or 2 and m, R¹, R², R³ and R⁴ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl, —R⁹—OR⁶, heterocyclyl, or heterocyclylalkyl;
each R⁹ is independently a direct bond;
R¹⁰ is a straight or branched alkylene chain; and
R¹¹ is hydrogen, alkyl, alkoxyalkyl, or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) selected from the following:
N-(2-chloropyrimidin-5-yl)-1,7-naphthyridin-8-amine;
N-(2-chloropyrimidin-5-yl)-3-((1-methyl-1H-pyrazol-4-yl)methoxy)-1,7-naphthyridin-8-amine;
1-(((8-((2-chloropyrimidin-5-yl)amino)-1,7-naphthyridin-3-yl)oxy)methyl)cyclopropane-1-carbonitrile;
3-(cyclopropylmethoxy)-N-(2-methylpyrimidin-5-yl)-1,7-naphthyridin-8-amine; and
3-((1-methyl-1H-pyrazol-4-yl)methoxy)-N-(2-methylpyrimidin-5-yl)-1,7-naphthyridin-8-amine;
as an individual stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein [symbol] is S-heteroaryl having one of the following formula (Id) or formula (Ie):

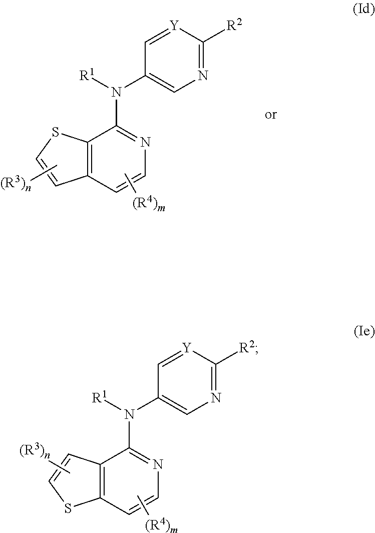

wherein n is 0, 1 or 2 and m, Y, R¹, R², R³ and R⁴ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein Y is ═C(R⁵)— having one of the following formula (Id1) or formula (Ie1):

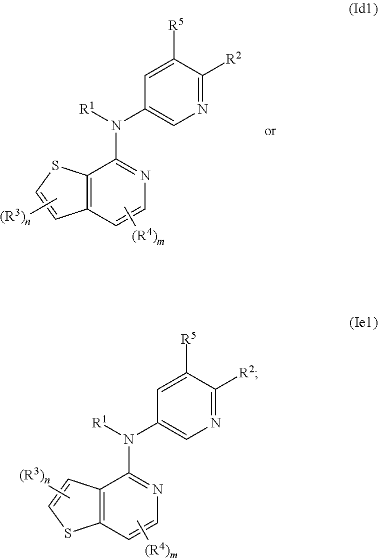

wherein n is 0, 1 or 2 and m, R¹, R², R³, R⁴ and R⁵ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl, —R⁹—OR⁶, heterocyclyl, or heterocyclylalkyl;
each R⁹ is independently a direct bond;
R¹⁰ is a straight or branched alkylene chain; and
R¹¹ is hydrogen, alkyl, alkoxyalkyl, or haloalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) selected from the following:
N-(6-chloropyridin-3-yl)-3-methylthieno[2,3-c]pyridin-7-amine;
N-(6-chloropyridin-3-yl)thieno[2,3-c]pyridin-7-amine; and
N-(6-chloropyridin-3-yl)thieno[3,2-c]pyridin-4-amine;
as an individual stereoisomer, enantiomer or tautomer thereof or a mixture thereof;
or a pharmaceutically acceptable salt, solvate or prodrug thereof.

Another embodiment of a compound of Formula (I), as described above in the Brief Summary, is a compound of Formula (I) wherein Y is ═N— having one of the following formula (Id2) or formula (Ie2):

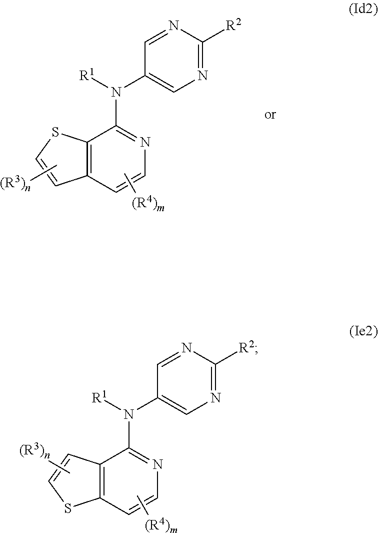

wherein n is 0, 1 or 2 and m, R¹, R², R³ and R⁴ are each as described above in the Brief Summary;
   as a stereoisomer, enantiomer, or tautomer thereof or a mixture thereof; or a pharmaceutically acceptable salt, solvate or prodrug thereof. Another embodiment of a compound of Formula (I), as described above in the Brief Summary, is a compound of Formula (I) wherein [symbol] is O-heteroaryl having one of the following formula (If) or formula (Ig):

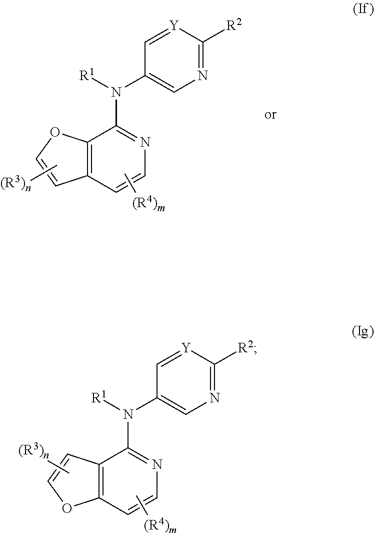

wherein n is 0, 1 or 2 and m, Y, R¹, R², R³ and R⁴ are each as described above in the Brief Summary;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein Y is ═C(R⁵)— having one of the following formula (If1) or formula (Ig1):

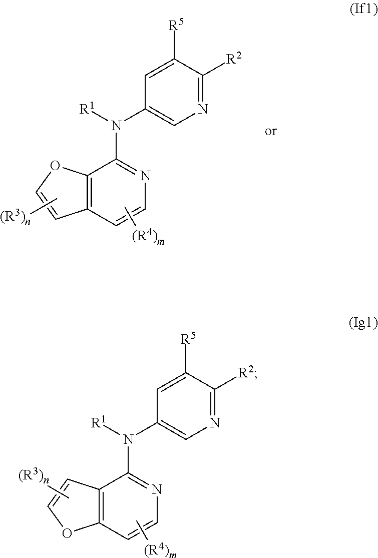

wherein n is 0, 1 or 2 and m, R¹, R², R³, R⁴ and R⁵ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl, —R⁹—OR⁶, heterocyclyl, or heterocyclylalkyl;
each R⁹ is independently a direct bond;
R¹⁰ is a straight or branched alkylene chain; and
R¹¹ is hydrogen, alkyl, alkoxyalkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) selected from the following:
N-(6-chloropyridin-3-yl)furo[2,3-c]pyridin-7-amine; and
N-(6-chloropyridin-3-yl)furo[3,2-c]pyridin-4-amine; as an individual stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein Y is ═N— having one of the following formula (If2) or formula (Ig2):

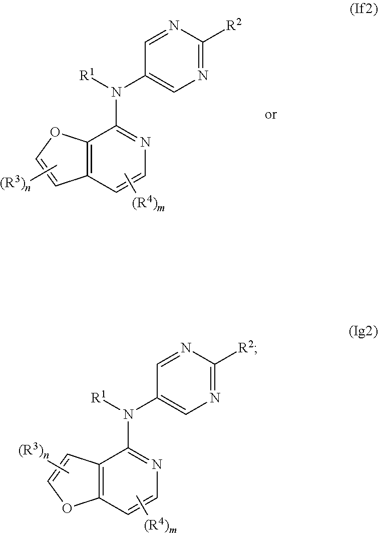

wherein n is 0, 1 or 2 and m, R¹, R², R³ and R⁴ are each as described above in the Brief Summary;
   as a stereoisomer, enantiomer, or tautomer thereof or a mixture thereof; or a pharmaceutically acceptable salt, solvate or prodrug thereof. Another embodiment of a compound of Formula (I), as described above in the Brief Summary, is a compound of Formula (I) wherein [symbol] is S,N-heteroaryl having the following formula (Ih):

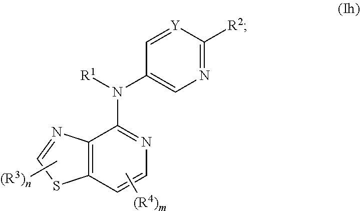

wherein n is 0 or 1 and m, Y, R¹, R², R³ and R⁴ are each as described above in the Brief Summary;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein Y is ═C(R⁵)— having the following formula (Ih1):

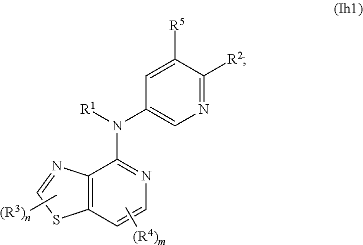

wherein n is 0 or 1 and m, n, R¹, R², R³, R⁴ and R⁵ are as described above in the Brief Summary;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, cycloalkyl, cycloalkylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen, alkyl or cycloalkylalkyl;
R² is hydrogen, alkyl, halo, haloalkyl, cycloalkyl or cycloalkylalkyl;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently alkyl or —R⁹—OR⁶;
R⁵ is hydrogen, halo, alkyl, haloalkyl, cycloalkyl or cycloalkylalkyl;
each R⁶ is independently hydrogen, —R¹⁰—OR¹¹, alkyl, haloalkyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond;
R¹⁰ is a straight or branched alkylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl;
each R⁹ is independently a direct bond;
R¹⁰ is a straight or branched alkylene chain; and
R¹¹ is hydrogen, alkyl or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) wherein:
m is 0 or 1;
n is 0, 1 or 2;
R¹ is hydrogen;
R² is alkyl or halo;
each R³ is independently alkyl, halo, cyanoalkyl, —R⁹—OR⁶, —R⁹—N(R⁶)₂, —R⁹—C(O)OR⁶ or —N═S(O)(R⁷)R⁸;
each R⁴ is independently —R⁹—OR⁶ or alkyl;
R⁵ is hydrogen or alkyl;
each R⁶ is independently —R¹⁰—OR¹¹, alkyl, haloalkyl, cycloalkylalkyl, aryl, heterocyclylalkyl or heteroarylalkyl;
R⁷ and R⁸ are each independently alkyl, —R⁹—OR⁶, heterocyclyl, or heterocyclylalkyl;
each R⁹ is independently a direct bond;
R¹⁰ is a straight or branched alkylene chain; and
R¹¹ is hydrogen, alkyl, alkoxyalkyl, or haloalkyl;
as a stereoisomer, enantiomer or tautomer thereof or a mixture thereof; or a pharmaceutically acceptable salt, solvate or prodrug thereof.

Another embodiment of a compound of Formula (I), as described above in the Brief Summary, is a compound of Formula (I) that is N-(6-chloropyridin-3-yl)thiazolo[4,5-c]pyridin-4-amine;
as an individual stereoisomer, enantiomer, or tautomer thereof or a mixture thereof; or a pharmaceutically acceptable salt, solvate or prodrug thereof.

Another embodiment of a compound of Formula (I), as described above in the Brief Summary, is a compound of Formula (c) wherein Y is ═N— having the following formula (h2):

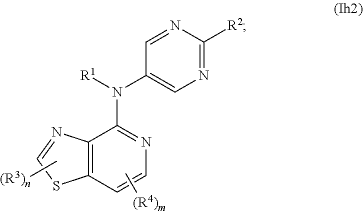

wherein n is 0 or 1 and m, R¹, R², R³ and R⁴ are each as described above in the Brief Summary;
as a stereoisomer, enantiomer, or tautomer thereof or a mixture thereof; or a pharmaceutically acceptable salt, solvate or prodrug thereof.

One embodiment provides a compound from Table 1 below as a stereoisomer, enantiomer or tautomer thereof or a mixture thereof; or a pharmaceutically acceptable salt, solvate or prodrug thereof.

TABLE 1
Representative compounds of Formula (I)

| Ex. | | |
|---|---|---|
| No. | Compound Structure | Compound Name |
| 1 | 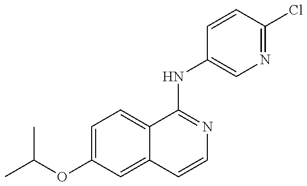 [SMILES: CC(C)Oc1ccc2c(Nc3ccc(Cl)nc3)nccc2c1] | N-(6-chloropyridin-3-yl)-6- isopropoxyisoquinolin-1- amine |
| 2 | 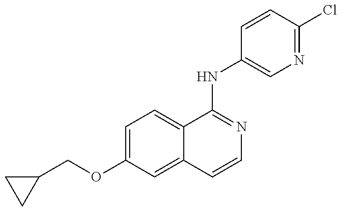 [SMILES: Clc1ccc(Nc2nccc3cc(OCC4CC4)ccc23)cn1] | N-(6-chloropyridin-3-yl)-6- (cyclopropylmethoxy)isoqiono- lin-1-amine |
| 3 | 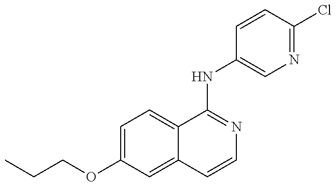 [SMILES: CCCOc1ccc2c(Nc3ccc(Cl)nc3)nccc2c1] | N-(6-chloropyridin-3-yl)-6- propoxyisoquinolin-1-amine |
| 4 | 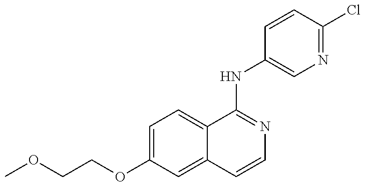 [SMILES: COCCOc1ccc2c(Nc3ccc(Cl)nc3)nccc2c1] | N-(6-chloropyridin-3-yl)-6-(2- methoxyethoxy)isoquinolin-1- amine |
| 5 | 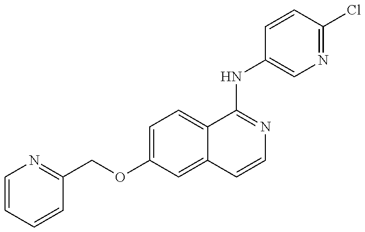 [SMILES: Clc1ccc(Nc2nccc3cc(OCc4ccccn4)ccc23)cn1] | N-(6-chlorpyridin-3-yl)-6- (pyridin-2- ylmethoxy)isoquinolin-1- amine |
| 6 | 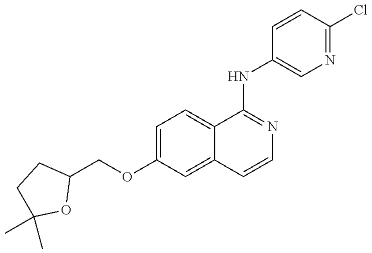 [SMILES: CC1(C)CCC(COc2ccc3c(Nc4ccc(Cl)nc4)nccc3c2)O1] | N-(6-chloropyridin-3-yl)-6- ((5,5-dimethyltetrahydrofuran- 2-yl)methoxy)isoquinolin-1- amine |
| 7 | 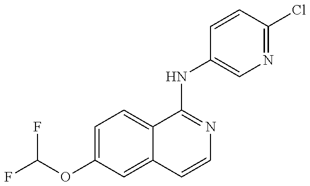 [SMILES: FC(F)Oc1ccc2c(Nc3ccc(Cl)nc3)nccc2c1] | N-(6-chloropyridin-3-yl)-6- (difluoromethoxy)isoquinolin- 1-amine |
| 8 | 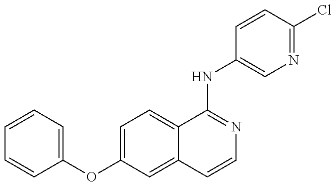 [SMILES: Clc1ccc(Nc2nccc3cc(Oc4ccccc4)ccc23)cn1] | N-(6-chloropyridin-3-yl)-6- phenoxyisoquinolin-1-amine |
| 9 | 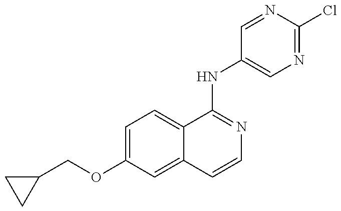 [SMILES: Clc1ncc(Nc2nccc3cc(OCC4CC4)ccc23)cn1] | N-(2-chloropyrimidin-5-yl)-6- (cyclopropylmethoxy)isoquino- line-1-amine |
| 10 | 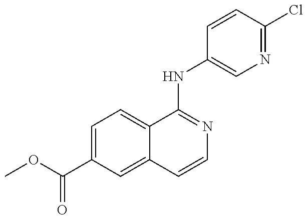 [SMILES: COC(=O)c1ccc2c(Nc3ccc(Cl)nc3)nccc2c1] | methyl 1-((6-chloropyridin-3- yl)amino)isoquinoline-6- carboxylate |
| 11 | 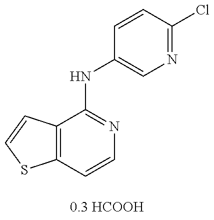 [SMILES: Clc1ccc(Nc2nccc3sccc23)cn1] | N-(6-chloropyridin-3- yl)thieno[3,2-c]pyridin-4- amine formate |
| 12 | 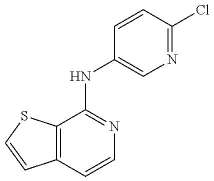 [SMILES: Clc1ccc(Nc2nccc3ccsc23)cn1] | N-(6-chloropyridin-3- yl)thieno[2,3-c]pyridin-7- amine |
| 13 | 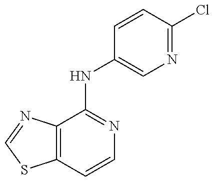 [SMILES: Clc1ccc(Nc2nccc3scnc23)cn1] | N-(6-chloropyridin-3- yl)thiazolo[4,5-c]pyridin-4- amine |
| 14 | 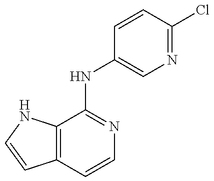 [SMILES: Clc1ccc(Nc2nccc3cc[nH]c23)cn1] | N-(6-chloropyridin-3-yl)-1H- pyrrolo[2,3-c]pyridin-7-amine |
| 15 | 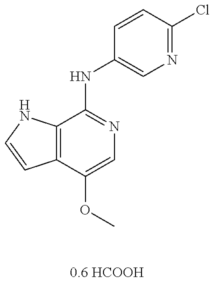 [SMILES: COc1cnc(Nc2ccc(Cl)nc2)c2[nH]ccc12] | N-(6-chloropyridin-3-yl)-4- methoxy-1H-pyrrolo[2,3- c]pyridin-7-amine formate |
| 16 | 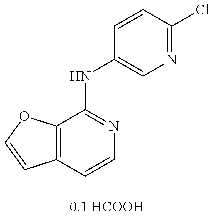 [SMILES: Clc1ccc(Nc2nccc3ccoc23)cn1] | N-(6-chloropyridin-3- yl)furo[2,3-c]pyridin-7-amine formate |
| 17 | 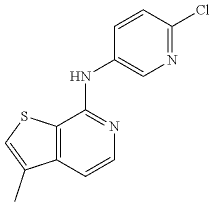 [SMILES: Cc1csc2c(Nc3ccc(Cl)nc3)nccc12] | N-(6-chloropyridin-3-yl)-3- methylthieno[2,3-c]pyridin-7- amine |
| 18 | 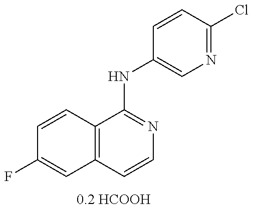 [SMILES: Fc1ccc2c(Nc3ccc(Cl)nc3)nccc2c1] | N-(6-chloropyridin-3-yl)-6- fluoroisoquinolin-1-amine formate |
| 19 | 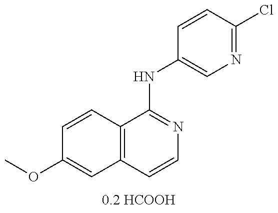 [SMILES: COc1ccc2c(Nc3ccc(Cl)nc3)nccc2c1] | N-(6-chloropyridin-3-yl)-6- methoxyisoquinolin-1-amine formate |
| 20 | 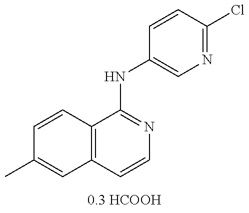 [SMILES: Cc1ccc2c(Nc3ccc(Cl)nc3)nccc2c1] | N-(6-chloropyridin-3-yl)-6- methylisoquinolin-1-amine formate |
| 21 | 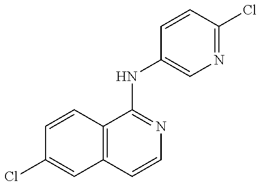 [SMILES: Clc1ccc2c(Nc3ccc(Cl)nc3)nccc2c1] | 6-chloro-N-(6-chloropyridin-3- yl)isoquinolin-1-amine |
| 22 | 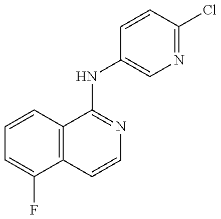 [SMILES: Fc1cccc2c(Nc3ccc(Cl)nc3)nccc12] | N-(6-chloropyridin-3-yl)-5- fluoroisoquinolin-1-amine |
| 23 | 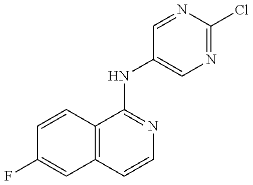 [SMILES: Fc1ccc2c(Nc3cnc(Cl)nc3)nccc2c1] | N-(2-chloropyrimidin-5-yl)-6- fluoroisoquinolin-1-amine |
| 24 | 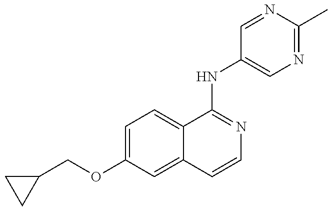 [SMILES: Cc1ncc(Nc2nccc3cc(OCC4CC4)ccc23)cn1] | 6-(cyclopropylmethoxy)-N-(2- methylpyrimidin-5- yl)isoquinolin-1-amine |
| 25 | 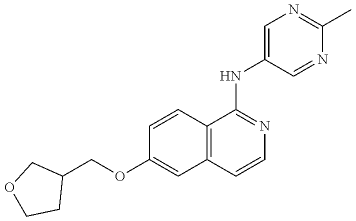 [SMILES: Cc1ncc(Nc2nccc3cc(OCC4CCOC4)ccc23)cn1] | N-(2-methylpyrimidin-5-yl)-6- ((tetrahydrofuran-3- yl)methoxy)isoquinolin-1- amine |
| 26 | 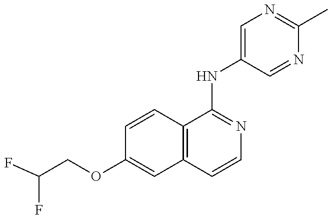 [SMILES: Cc1ncc(Nc2nccc3cc(OCC(F)F)ccc23)cn1] | 6-(2,2-difluoroethoxy)-N-(2- methylpyrimidin-5- yl)isoquinolin-1-amine |
| 27 | 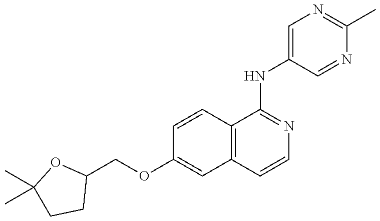 | 6-((5,5- dimethyltetrahydrofuran-2- yl)methoxy)-N-(2- methylpyrimidin-5- yl)isoquinolin-1-amine |
| 28 | 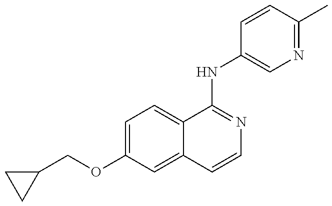 [SMILES: Cc1ccc(Nc2nccc3cc(OCC4CC4)ccc23)cn1] | 6-(cyclopropylmethoxy)-N-(6- methylpyridin-3-yl)isoquinolin- 1-amine |
| 29 | 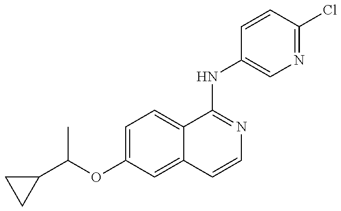 [SMILES: CC(Oc1ccc2c(Nc3ccc(Cl)nc3)nccc2c1)C1CC1] | N-(6-chloropyridin-3-yl)-6-(1- cyclopropylethoxy)isoquinolin- 1-amine |
| 30 | 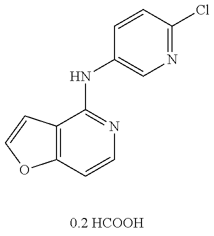 [SMILES: Clc1ccc(Nc2nccc3occc23)cn1] | N-(6-chloropyridin-3- yl)furo[3,2-c]pyridin-4-amine formate |
| 31 | 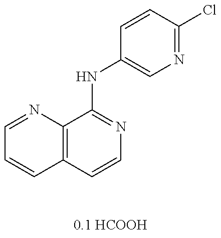 [SMILES: Clc1ccc(Nc2nccc3cccnc23)cn1] | N-(6-chloropyridin-3-yl)-1,7- naphthyridin-8-amine formate |
| 32 | 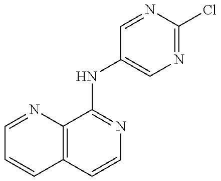 [SMILES: Clc1ncc(Nc2nccc3cccnc23)cn1] | N-(2-chloropyimidin-5-yl)-1,7- naphthyridin-8-amine |
| 33 | 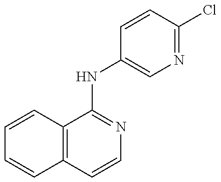 [SMILES: Clc1ccc(Nc2nccc3ccccc23)cn1] | N-(6-chloropyridin-3- yl)isoquinolin-1-amine |
| 34 | 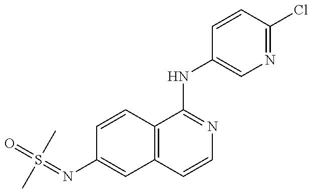 [SMILES: CS(C)(=O)=Nc1ccc2c(Nc3ccc(Cl)nc3)nccc2c1] | ((1-((6-chloropyridin-3- yl)amino)isoquinolin-6- yl)imino)dimethyl-λ⁶-sulfanone |
| 35 | 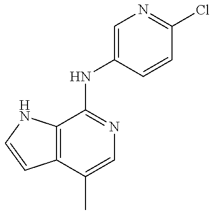 [SMILES: Cc1cnc(Nc2ccc(Cl)nc2)c2[nH]ccc12] | N-(6-chloropyridin-3-yl)-4- methyl-1H-pyrrolo[2,3- c]pyridin-7-amine |
| 36 | 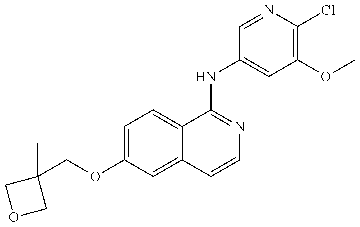 [SMILES: COc1cc(Nc2nccc3cc(OCC4(C)COC4)ccc23)cnc1Cl] | Synthesis of N-(6-chloro-5- methoxypyridin-3-yl)-6-((3- methyloxetan-3- yl)methoxy)isoquinolin-1- amine |
| 37 | 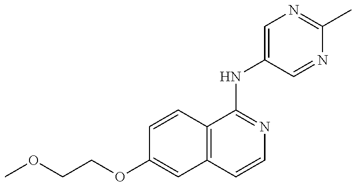 | 6-(2-methoxyethoxy)-N-(2- methylpyrimidin-5- yl)isoquinolin-1-amine |
| 38 | 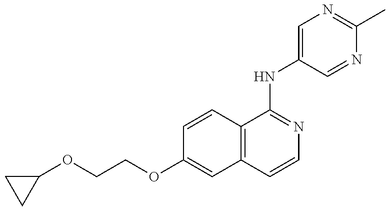 [SMILES: Cc1ncc(Nc2nccc3cc(OCCOC4CC4)ccc23)cn1] | 6-(2-cyclopropoxyethoxy)-N- (2-methylpyrimidin-5- yl)isoquinolin-1-amine |
| 39 | 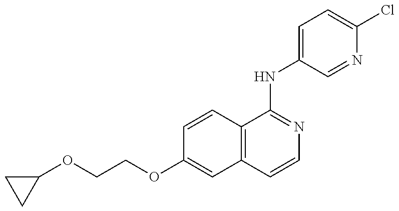 [SMILES: Clc1ccc(Nc2nccc3cc(OCCOC4CC4)ccc23)cn1] | N-(6-chloropyridin-3-yl)-6-(2- cyclopropoxyethoxy)isoquinolin- 1-amine |
| 40 | 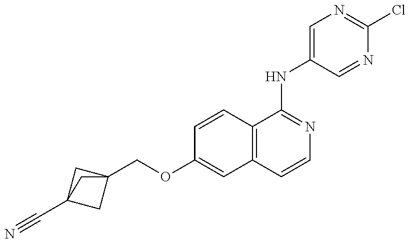 [SMILES: N#CC12CC(COc3ccc4c(Nc5cnc(Cl)nc5)nccc4c3)(C1)C2] | 3-(((1-((2-chloropyrimidin-5- yl)amino)isoquinolin-6- yl)oxy)methyl)bicyclo[1.1.1]pen- tane-1-carbonitrile |
| 41 | 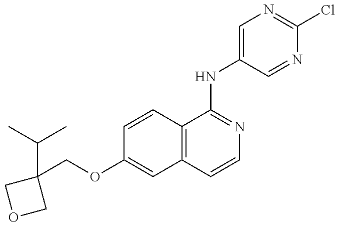 [SMILES: CC(C)C1(COc2ccc3c(Nc4cnc(Cl)nc4)nccc3c2)COC1] | N-(2-chloropyrimidin-5-yl)-6- ((3-isopropyloxetan-3- yl)methoxy)isoquinoln-1- amine |
| 42 | 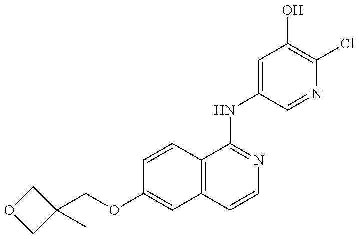 [SMILES: CC1(COc2ccc3c(Nc4cnc(Cl)c(O)c4)nccc3c2)COC1] | 2-chloro-5-((6-((3- methyloxetan-3- yl)methoxy)isoquinolin-1- yl)amino)pyridin-3-ol |
| 43 | 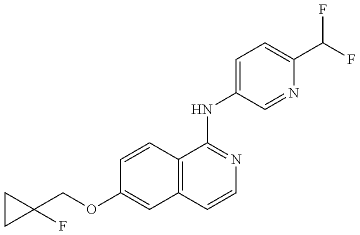 [SMILES: FC(F)c1ccc(Nc2nccc3cc(OCC4(F)CC4)ccc23)cn1] | N-(6-(difluoromethyl)pyridin-3- yl)-6-((1- fluorocyclopropyl)methoxy)iso- quinolin-1-amine |
| 44 | 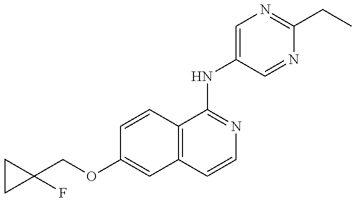 [SMILES: CCc1ncc(Nc2nccc3cc(OCC4(F)CC4)ccc23)cn1] | N-(2-ethylpyrimidin-5-yl)-6- ((1- fluorocyclopropyl)methoxy)iso- quinolin-1-amine |
| 45 | 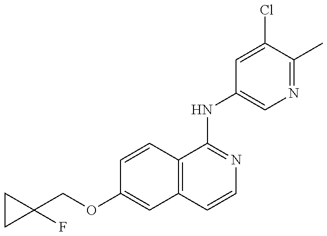 [SMILES: Cc1ncc(Nc2nccc3cc(OCC4(F)CC4)ccc23)cc1Cl] | N-(5-chloro-6-methylpyridin-3- yl)-6-((1- fluorocyclopropyl)methoxy)iso- quinolin-1-amine |
| 46 | 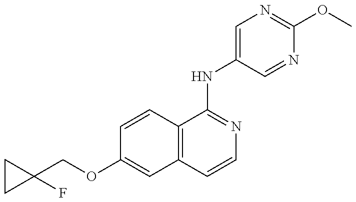 [SMILES: COc1ncc(Nc2nccc3cc(OCC4(F)CC4)ccc23)cn1] | 6-((1- fluorocyclopropyl)methoxy)-N- (2-methoxypyrimidin-5- yl)isoquinolin-1-amine |
| 47 | 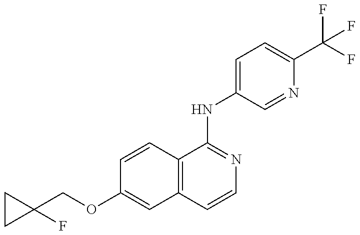 [SMILES: FC1(COc2ccc3c(Nc4ccc(C(F)(F)F)nc4)nccc3c2)CC1] | 6-((1- fluorocyclopropyl)methoxy)-N- (6-(trifluoromethyl)pyridin-3- yl)isoquinolin-1-amine |
| 48 | 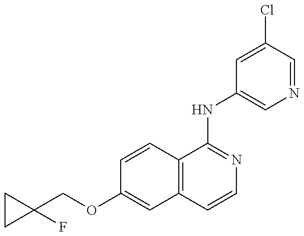 [SMILES: FC1(COc2ccc3c(Nc4cncc(Cl)c4)nccc3c2)CC1] | N-(5-chloropyridin-3-yl)-6-((1- fluorocyclopropyl)methoxy)iso- quinolin-1-amine |
| 49 | 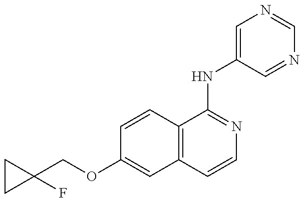 [SMILES: FC1(COc2ccc3c(Nc4cncnc4)nccc3c2)CC1] | 6-((1- fluorocyclopropyl)methoxy)-N- (pyrimidin-5-yl)isoquinolin-1- amine |
| 50 | 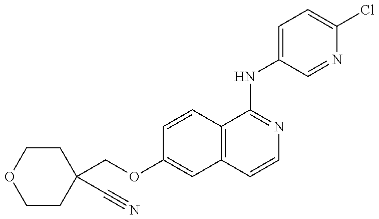 [SMILES: N#CC1(COc2ccc3c(Nc4ccc(Cl)nc4)nccc3c2)CCOCC1] | 4-(((1-((6-chloropyridin-3- yl)amino)isoquinolin-6- yl)oxy)methyl)tetrahydro-2H- pyran-4-carbonitrile |
| 51 | 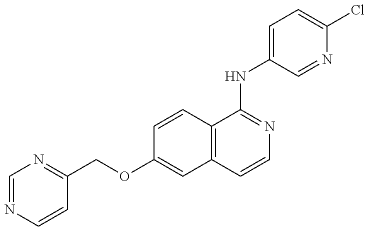 [SMILES: Clc1ccc(Nc2nccc3cc(OCc4ccncn4)ccc23)cn1] | N-(6-chloropyridin-3-yl)-6- (pyrimidin-4- ylmethoxy)isoquinolin-1- amine |
| 52 | 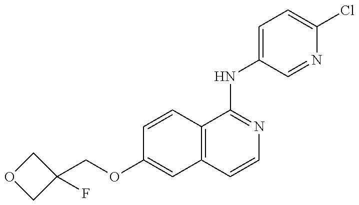 [SMILES: FC1(COc2ccc3c(Nc4ccc(Cl)nc4)nccc3c2)COC1] | N-(6-chloropyridin-3-yl)-6-((3- fluorooxetan-3- yl)methoxy)isoquinolin-1- amine |
| 53 | 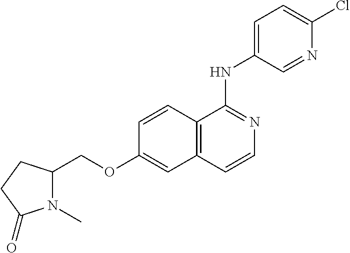 [SMILES: CN1C(=O)CCC1COc1ccc2c(Nc3ccc(Cl)nc3)nccc2c1] | 5-(((1-((6-chloropyridin-3- yl)amino)isoquinolin-6- yl)oxy)methyl)-1- methylpyrrolidin-2-one |
| 54 | 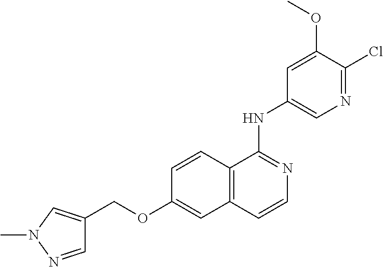 [SMILES: COc1cc(Nc2nccc3cc(OCc4cnn(C)c4)ccc23)cnc1Cl] | N-(6-chloro-5-methoxypyridin- 3-yl)-6-((1-methyl-1H-pyrazol- 4-yl)methoxy)isoquinolin-1- amine |
| 55 | 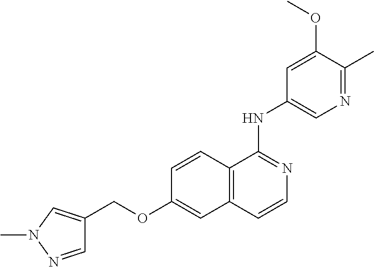 | N-(5-methoxy-6- methylpyridin-3-yl)-6-((1- methyl-1H-pyrazol-4- yl)methoxy)isoquinolin-1- amine |
| 56 | 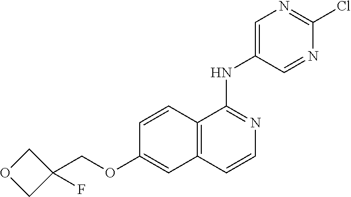 [SMILES: FC1(COc2ccc3c(Nc4cnc(Cl)nc4)nccc3c2)COC1] | N-(2-chloropyrimidin-5-yl)-6- ((3-fluorooxetan-3- yl)methoxy)isoquinolin-1- amine |
| 57 | 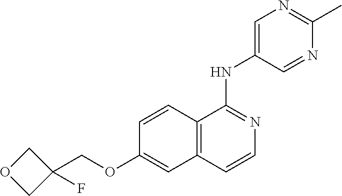 [SMILES: Cc1ncc(Nc2nccc3cc(OCC4(F)COC4)ccc23)cn1] | 6-((3-fluorooxetan-3- yl)methoxy)-N-(2- methylpyrimidin-5- yl)isoquinolin-1-amine |
| 58 | 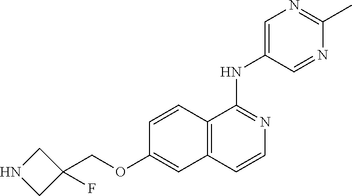 [SMILES: Cc1ncc(Nc2nccc3cc(OCC4(F)CNC4)ccc23)cn1] | 6-((3-fluoroazetidin-3- yl)methoxy)-N-(2- methylpyrimidin-5- yl)isoquinolin-1-amine |
| 59 | 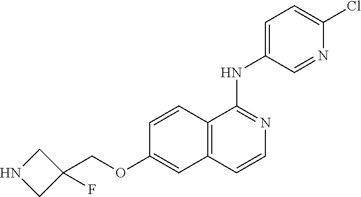 [SMILES: FC1(COc2ccc3c(Nc4ccc(Cl)nc4)nccc3c2)CNC1] | N-(6-chloropyridin-3-yl)-6-((3- fluoroazetidin-3- yl)methoxy)isoquinolin-1- amine |
| 60 | 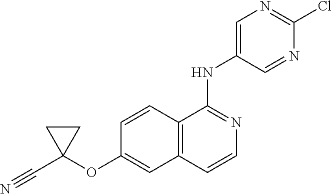 [SMILES: N#CC1(Oc2ccc3c(Nc4cnc(Cl)nc4)nccc3c2)CC1] | 1-((1-((2-chloropyrimidin-5- yl)amino)isoquinolin-6- yl)oxy)cyclopropane-1- carbonitrile |
| 61 | 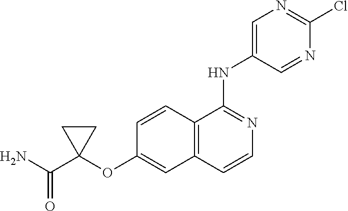 [SMILES: NC(=O)C1(Oc2ccc3c(Nc4cnc(Cl)nc4)nccc3c2)CC1] | 1-((1-((2-chloropyrimidin-5- yl)amino)isoquinolin-6- yl)oxy)cyclopropane-1- carboxamide |
| 62 | 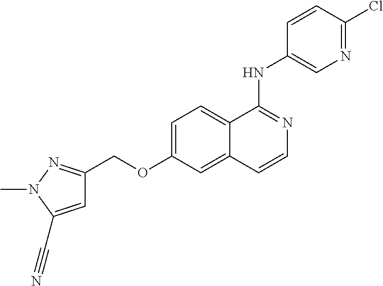 [SMILES: Cn1nc(COc2ccc3c(Nc4ccc(Cl)nc4)nccc3c2)cc1C#N] | 3-(((1-((6-chloropyridin-3- yl)amino)isoquinolin-6- yl)oxy)methyl)-1-methyl-1H- pyrazole-5-carbonitrile |
| 63 | 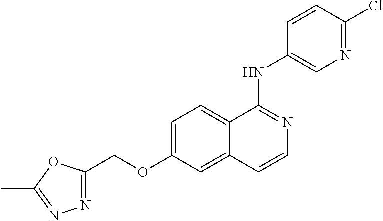 [SMILES: Cc1nnc(COc2ccc3c(Nc4ccc(Cl)nc4)nccc3c2)o1] | N-(6-chloropyridin-3-yl)-6-((5- methyl-1,3,4-oxadiazol-2- yl)methoxy)isoquinolin-1- amine |
| 64 | 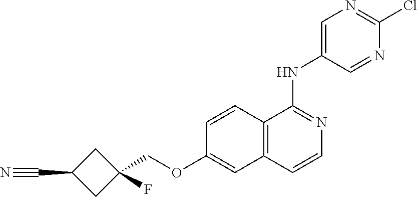 [SMILES: N#C[C@H]1C[C@](F)(COc2ccc3c(Nc4cnc(Cl)nc4)nccc3c2)C1] | (1s,3s)-3-(((1-((2- chloropyrimidin-5- yl)amino)isoquinolin-6- yl)oxy)methyl)-3- fluorocyclobutane-1- carbonitrile |
| 65 | 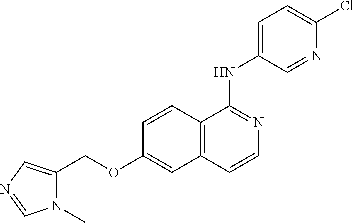 [SMILES: Cn1cncc1COc1ccc2c(Nc3ccc(Cl)nc3)nccc2c1] | N-(6-chloropyridin-3-yl)-6-((1- methyl-1H-imidazol-5- yl)methoxy)isoquinolin-1- amine |
| 66 | 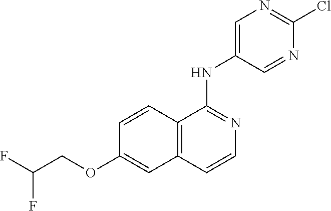 [SMILES: FC(F)COc1ccc2c(Nc3cnc(Cl)nc3)nccc2c1] | N-(2-chloropyrimidin-5-yl)-6- (2,2- difluoroethoxy)isoquinolin-1- amine |
| 67 | 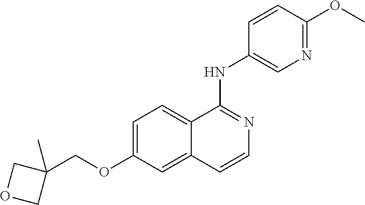 [SMILES: COc1ccc(Nc2nccc3cc(OCC4(C)COC4)ccc23)cn1] | N-(6-methoxypyridin-3-yl)-6- ((3-methyloxetan-3- yl)methoxy)isoquinolin-1- amine |
| 68 | 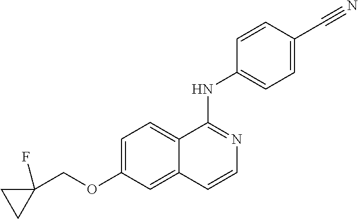 [SMILES: N#Cc1ccc(Nc2nccc3cc(OCC4(F)CC4)ccc23)cc1] | N-(2-chloropyrimidin-5-yl)-6- ((1- fluorocyclopropyl)methoxy)iso- quinolin-1-amine |
| 69 | 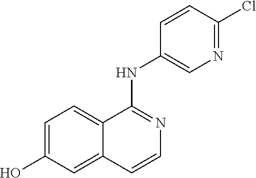 [SMILES: Oc1ccc2c(Nc3ccc(Cl)nc3)nccc2c1] | 1-((6-chloropyridin-3- yl)amino)isoquinolin-6-ol |
| 70 | 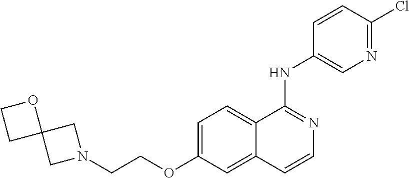 [SMILES: Clc1ccc(Nc2nccc3cc(OCCN4CC5(CCO5)C4)ccc23)cn1] | 6-(2-(1-oxa-6- azaspiro[3.3]heptan-6- yl)ethoxy)-N-(6-chloropyridin- 3-yl)isoquinolin-1-amine |
| 71 | 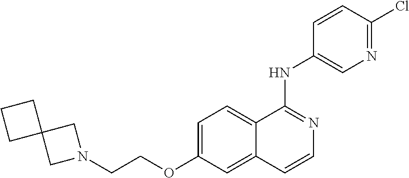 [SMILES: Clc1ccc(Nc2nccc3cc(OCCN4CC5(CCC5)C4)ccc23)cn1] | 6-(2-(2-azaspiro[3.3]heptan-2- yl)ethoxy)-N-(6-chloropyridin- 3-yl)isoquinolin-1-amine |
| 72 | 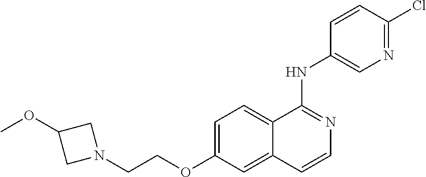 [SMILES: COC1CN(CCOc2ccc3c(Nc4ccc(Cl)nc4)nccc3c2)C1] | N-(6-chloropyridin3--yl)-6-(2- (3-methoxyazetidin-1- yl)ethoxy)isoquinolin-1-amine |
| 73 | 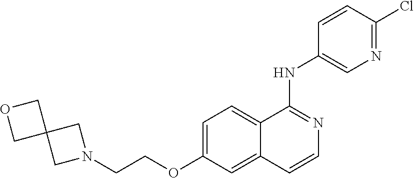 [SMILES: Clc1ccc(Nc2nccc3cc(OCCN4CC5(COC5)C4)ccc23)cn1] | 6-(2-(2-oxa-6- azaspiro[3.3]heptan-6- yl)ethoxy)-N-(6-chloropyridin- 3-yl)isoquinolin-1-amine |
| 74 | 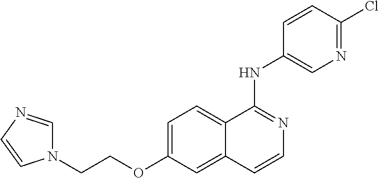 [SMILES: Clc1ccc(Nc2nccc3cc(OCCn4ccnc4)ccc23)cn1] | 6-(2-(1H-imidazol-1- yl)ethoxy)-N-(6-chloropyridin- 3-yl)isoquinolin-1-amine |
| 75 | 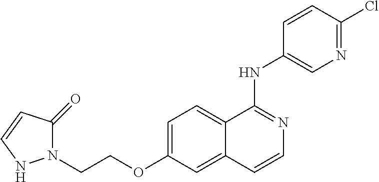 [SMILES: O=c1cc[nH]n1CCOc1ccc2c(Nc3ccc(Cl)nc3)nccc2c1] | 2-(2-((1-((6-chloropyridin-3- yl)amino)isoquinolin-6- yl)oxy)ethyl)-1,2-dihydro-3H- pyrazol-3-one |
| 76 | 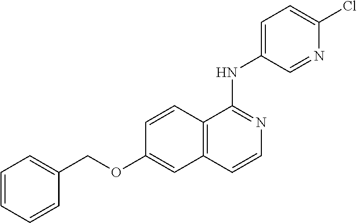 [SMILES: Clc1ccc(Nc2nccc3cc(OCc4ccccc4)ccc23)cn1] | N-(6-chloropyridin-3-yl)-6- fluoro-N-((2- (trimethylsilyl)ethoxy)- methyl)isoquinolin-1-amine |
| 77 | 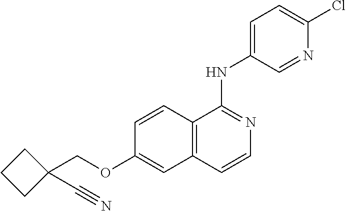 [SMILES: N#CC1(COc2ccc3c(Nc4ccc(Cl)nc4)nccc3c2)CCC1] | 1-(((1-((6-chloropyridin-3- yl)amino)isoquinolin-6- yl)oxy)methyl)cyclobutane-1- carbonitrile |
| 78 | 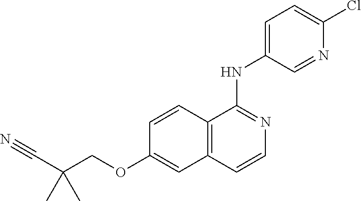 [SMILES: CC(C)(C#N)COc1ccc2c(Nc3ccc(Cl)nc3)nccc2c1] | 3-((1-((6-chloropyridin-3- yl)amino)isoquinolin-6-yl)oxy)- 2,2-dimethylpropanenitrile |
| 79 | 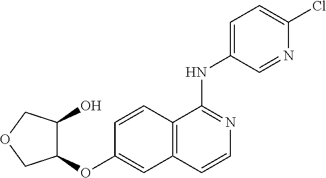 [SMILES: O[C@@H]1COC[C@@H]1Oc1ccc2c(Nc3ccc(Cl)nc3)nccc2c1] | rac-(3R,4S)-4-((1-((6- chloropyridin-3- yl)amino)isoquinolin-6- yl)oxy)tetrahydrofuran-3-ol |
| 80 | 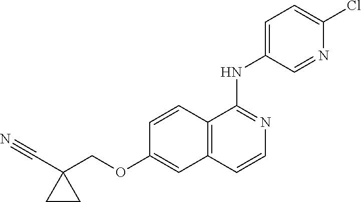 [SMILES: N#CC1(COc2ccc3c(Nc4ccc(Cl)nc4)nccc3c2)CC1] | 1-(((1-((6-chloropyridin-3- yl)amino)isoquinolin-6- yl)oxy)methyl)cyclopropane- 1-carbonitrile |
| 81 | 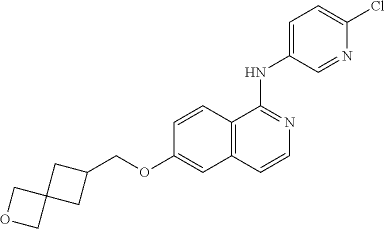 [SMILES: Clc1ccc(Nc2nccc3cc(OCC4CC5(COC5)C4)ccc23)cn1] | 6-((2-oxaspiro[3.3]heptan-6- yl)methoxy)-N-(6- chloropyridin-3-yl)isoquinolin- 1-amine |
| 82 | 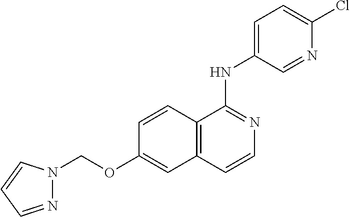 [SMILES: Clc1ccc(Nc2nccc3cc(OCn4cccn4)ccc23)cn1] | 6-((1H-pyrazol-1-yl)methoxy)- N-(6-chloropyridin-3- yl)isoquinolin-1-amine |
| 83 | 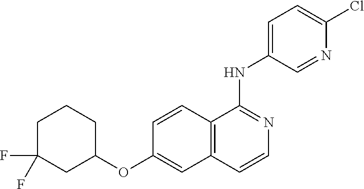 [SMILES: FC1(F)CCCC(Oc2ccc3c(Nc4ccc(Cl)nc4)nccc3c2)C1] | N-(6-chloropyridin-3-yl)-6- ((3,3- difluorocyclohexyl)oxy)isoquin- olin-1-amine |
| 84 | 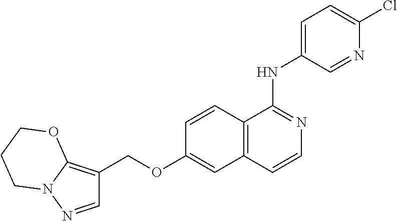 [SMILES: Clc1ccc(Nc2nccc3cc(OCc4cnn5c4OCCC5)ccc23)cn1] | N-(6-chloropyridin-3-yl)-6- ((6,7-dihydro-5H- pyrazolo[5,1-b][1,3]oxazin-3- yl)methoxy)isoquinolin-1- amine |
| 85 | 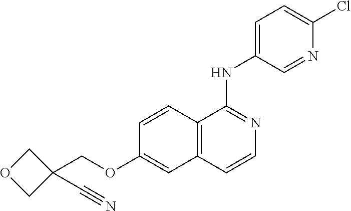 [SMILES: N#CC1(COc2ccc3c(Nc4ccc(Cl)nc4)nccc3c2)COC1] | 3-(((1-((6-chloropyridin-3- yl)amino)isoquinolin-6- yl)oxy)methyl)oxetane-3- carbonitrile |
| 86 | 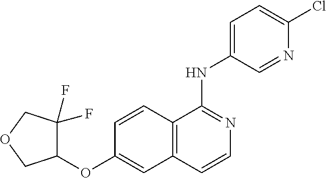 [SMILES: FC1(F)COCC1Oc1ccc2c(Nc3ccc(Cl)nc3)nccc2c1] | N-(6-chloropyridin-3-yl)-6- ((4,4-difluorotetrahydrofuran- 3-yl)oxy)isoquinolin-1-amine 2,2,2-trifluoroacetate |
| 87 | 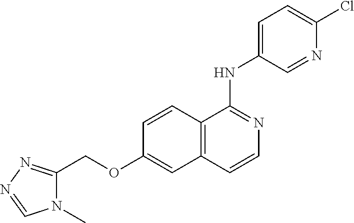 [SMILES: Cn1cnnc1COc1ccc2c(Nc3ccc(Cl)nc3)nccc2c1] | N-(6-chloropyridin-3-yl)-6-((4- methyl-4H-1,2,4-triazol-3- yl)methoxy)isoquinolin-1- amine |
| 88 | 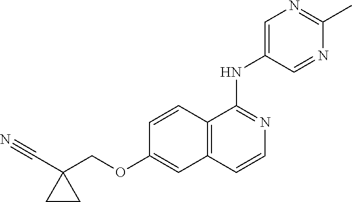 [SMILES: Cc1ncc(Nc2nccc3cc(OCC4(C#N)CC4)ccc23)cn1] | 1-(((1-((2-methylpyrimidin-5- yl)amino)isoquinolin-6- yl)oxy)methyl)cyclopropane- 1-carbonitrile |
| 89 | 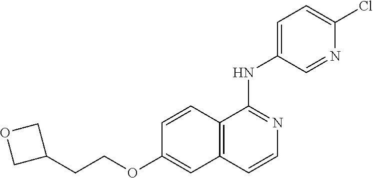 [SMILES: Clc1ccc(Nc2nccc3cc(OCCC4COC4)ccc23)cn1] | N-(6-chloropyridin-3-yl)-6-(2- (oxetan-3- yl)ethoxy)isoquinolin-1-amine |
| 90 | 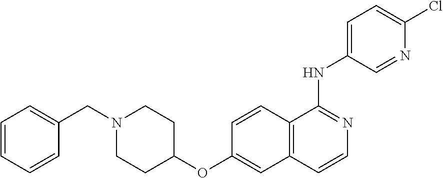 [SMILES: Clc1ccc(Nc2nccc3cc(OC4CCN(Cc5ccccc5)CC4)ccc23)cn1] | 6-((1-benzylpiperidin-4- yl)oxy)-N-(6-chloropyridin-3- yl)isoquinolin-1-amine |
| 91 | 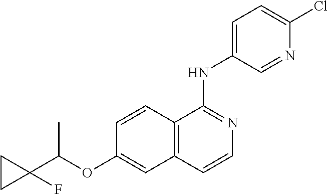 [SMILES: CC(Oc1ccc2c(Nc3ccc(Cl)nc3)nccc2c1)C1(F)CC1] | N-(6-chloropyridin-3-yl)-6-(1- (1- fluorocyclopropyl)ethoxy)iso- quinolin-1-amine |
| 92 | 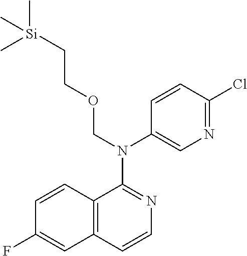 [SMILES: C[Si](C)(C)CCOCN(c1ccc(Cl)nc1)c1nccc2cc(F)ccc12] | N-(6-chloropyridin-3-yl)-6- fluoro-N-((2- (trimethylsilyl)ethoxy)- methyl)isoquinolin-1-amine |
| 93 | 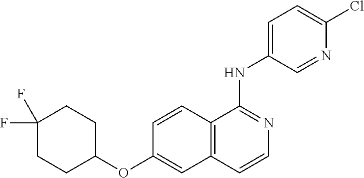 [SMILES: FC1(F)CCC(Oc2ccc3c(Nc4ccc(Cl)nc4)nccc3c2)CC1] | N-(6-chloropyridin-3-yl)-6- ((4,4- difluorocyclohexyl)oxy)iso- quinolin-1-amine |
| 94 | 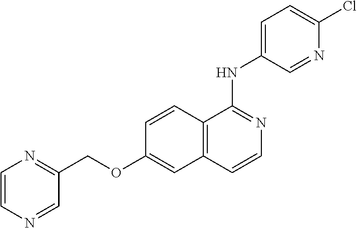 [SMILES: Clc1ccc(Nc2nccc3cc(OCc4cnccn4)ccc23)cn1] | N-(6-chloropyridin-3-yl)-6- (pyrazin-2- ylmethoxy)isoquinolin-1- amine |
| 95 | 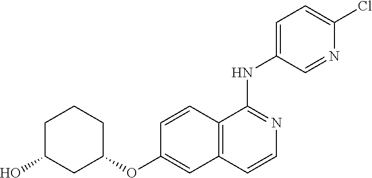 [SMILES: O[C@@H]1CCC[C@H](Oc2ccc3c(Nc4ccc(Cl)nc4)nccc3c2)C1] | rac-(1R,3S)-3-((1-((6- chloropyridin-3- yl)amino)isoquinolin-6- yl)oxy)cyclohexan-1-ol |
| 96 | 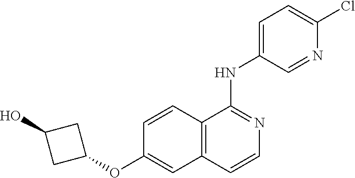 [SMILES: O[C@H]1C[C@H](Oc2ccc3c(Nc4ccc(Cl)nc4)nccc3c2)C1] | (1R,3R)-3-((1-((6- chloropyridin-3- yl)amino)isoquinolin-6- yl)oxy)cyclobutan-1-ol |
| 97 | 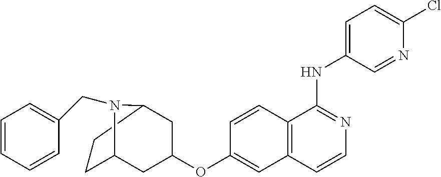 [SMILES: Clc1ccc(Nc2nccc3cc(OC4CC5CCC(C4)N5Cc4ccccc4)ccc23)cn1] | 6-((8-benzyl-8- azabicyclo[3.2.1]octan-3- yl)oxy)-N-(6-chloropyridin-3- yl)isoquinolin-1-amine |
| 98 | 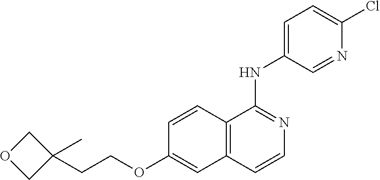 [SMILES: CC1(CCOc2ccc3c(Nc4ccc(Cl)nc4)nccc3c2)COC1] | N-(6-chloropyridin-3-yl)-6-(2- (3-methyloxetan-3- yl)ethoxy)isoquinolin-1-amine |
| 99 | 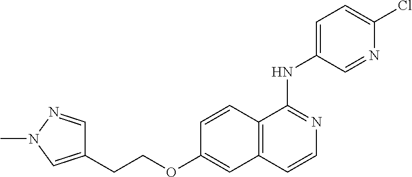 [SMILES: Cn1cc(CCOc2ccc3c(Nc4ccc(Cl)nc4)nccc3c2)cn1] | N-(6-chloropyridin-3-yl)-6-(2- (1-methyl-1H-pyrazol-4- yl)ethoxy)isoquinolin-1-amine |
| 100 | 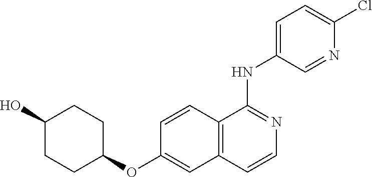 [SMILES: O[C@H]1CC[C@@H](Oc2ccc3c(Nc4ccc(Cl)nc4)nccc3c2)CC1] | Cis-4-((1-((6-chloropyridin-3- yl)amino)isoquinolin-6- yl)oxy)cyclohexan-1-ol |
| 101 | 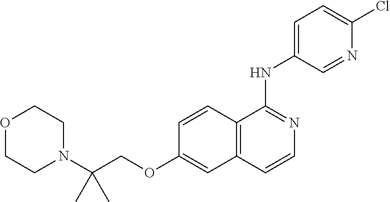 [SMILES: CC(C)(COc1ccc2c(Nc3ccc(Cl)nc3)nccc2c1)N1CCOCC1] | N-(6-chloropyridin-3-yl)-6-(2- methyl-2- morpholinopropoxy)isoquinolin- 1-amine |
| 102 | 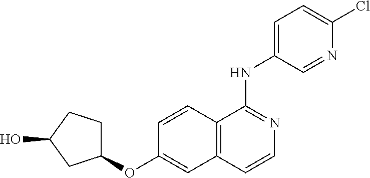 [SMILES: O[C@H]1CC[C@@H](Oc2ccc3c(Nc4ccc(Cl)nc4)nccc3c2)C1] | rac-(1S,3R)-3-((1-((6- chloropyridin-3- yl)amino)isoquinolin-6- yl)oxy)cyclopentan-1-ol |
| 103 | 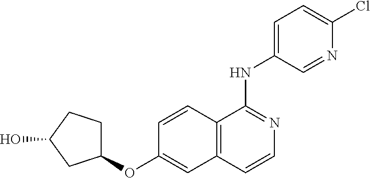 [SMILES: O[C@@H]1CC[C@@H](Oc2ccc3c(Nc4ccc(Cl)nc4)nccc3c2)C1] | rac-(1R,3R)-3-((1-((6- chloropyridin-3- yl)amino)isoquinolin-6- yl)oxy)cyclopentan-1-ol |
| 104 | 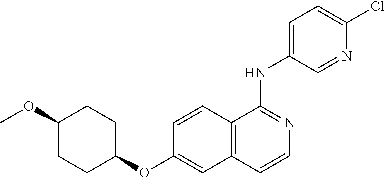 [SMILES: CO[C@H]1CC[C@@H](Oc2ccc3c(Nc4ccc(Cl)nc4)nccc3c2)CC1] | N-(6-chloropyridin-3-yl)-6- (((1s,4s)-4- methoxycyclohexyl)oxy)iso- quinolin-1-amine |
| 105 | 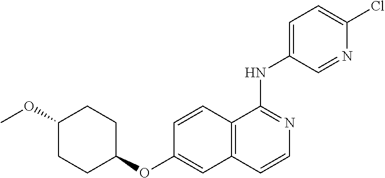 [SMILES: CO[C@H]1CC[C@H](Oc2ccc3c(Nc4ccc(Cl)nc4)nccc3c2)CC1] | N-(6-chloropyridin-3-yl)-6- (((1r,4r)-4- methoxycyclohexyl)oxy)iso- quinolin-1-amine |
| 106 | 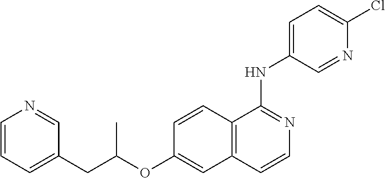 [SMILES: CC(Cc1cccnc1)Oc1ccc2c(Nc3ccc(Cl)nc3)nccc2c1] | N-(6-chloropyridin-3-yl)-6-((1- (pyridin-3-yl)propan-2- yl)oxy)isoquinolin-1-amine |
| 107 | 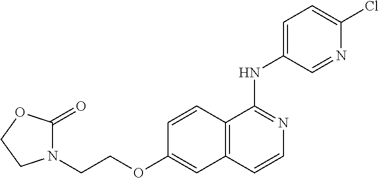 [SMILES: O=C1OCCN1CCOc1ccc2c(Nc3ccc(Cl)nc3)nccc2c1] | 3-(2-((1-((6-chloropyridin-3- yl)amino)isoquinolin-6- yl)oxy)ethyl)oxazolidin-2-one |
| 108 | 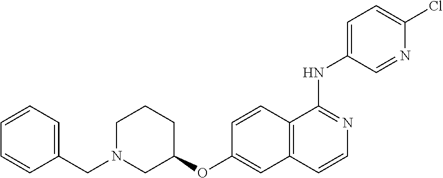 [SMILES: Clc1ccc(Nc2nccc3cc(O[C@@H]4CCCN(Cc5ccccc5)C4)ccc23)cn1] | (R)-6-((1-benzylpiperidin-3- yl)oxy)-N-(6-chloropyridin-3- yl)isoquinolin-1-amine |
| 109 | 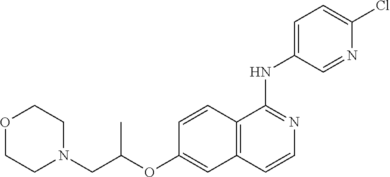 [SMILES: CC(CN1CCOCC1)Oc1ccc2c(Nc3ccc(Cl)nc3)nccc2c1] | N-(6-chloropyridin-3-yl)-6-((1- morpholinopropan-2- yl)oxy)isoquinolin-1-amine |
| 110 | 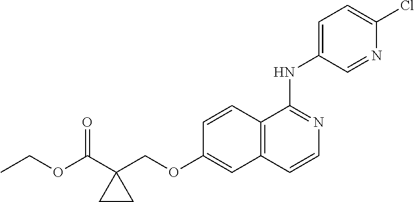 [SMILES: CCOC(=O)C1(COc2ccc3c(Nc4ccc(Cl)nc4)nccc3c2)CC1] | ethyl 1-(((1-((6-chloropyridin- 3-yl)amino)isoquinolin-6- yl)oxy)methyl)cyclopropane- 1-carboxylate |
| 111 | 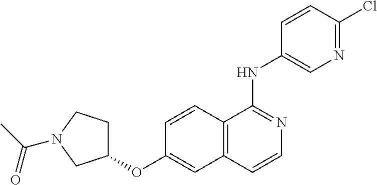 [SMILES: CC(=O)N1CC[C@H](Oc2ccc3c(Nc4ccc(Cl)nc4)nccc3c2)C1] | (S)-1-(3-((1-((6-chloropyridin- 3-yl)amino)isoquinolin-6- yl)oxy)pyrrolidin-1-yl)ethan-1- one |
| 112 | 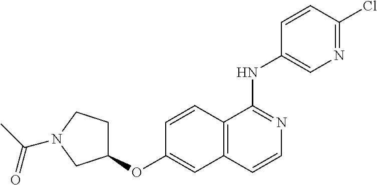 [SMILES: CC(=O)N1CC[C@@H](Oc2ccc3c(Nc4ccc(Cl)nc4)nccc3c2)C1] | (R)-1-(3-((1-((6-chloropyridin- 3-yl)amino)isoquinolin-6- yl)oxy)pyrrolidin-1-yl)ethan-1- one |
| 113 | 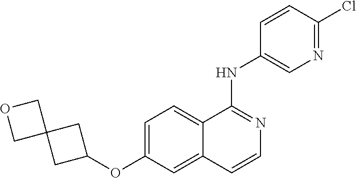 [SMILES: Clc1ccc(Nc2nccc3cc(OC4CC5(COC5)C4)ccc23)cn1] | 6-((2-oxaspiro[3.3]heptan-6- yl)oxy)-N-(6-chloropyridin-3- yl)isoquinolin-1-amine |
| 114 | 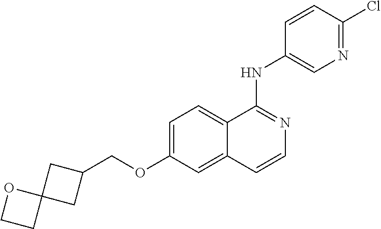 [SMILES: Clc1ccc(Nc2nccc3cc(OCC4CC5(CCO5)C4)ccc23)cn1] | 6-((1-oxaspiro[3.3]heptan-6- yl)methoxy)-N-(6- chloropyridin-3-yl)isoquinolin- 1-amine |
| 115 | 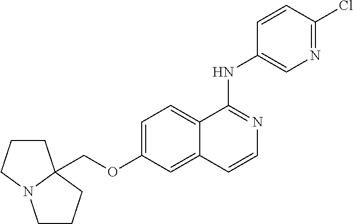 [SMILES: Clc1ccc(Nc2nccc3cc(OCC45CCCN4CCC5)ccc23)cn1] | N-(6-chloropyridin-3-yl)-6- ((tetrahydro-1H-pyrrolizin- 7a(5H)- yl)methoxy)isoquinolin-1- amine |
| 116 | 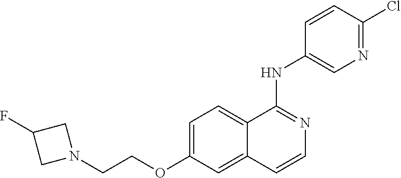 [SMILES: FC1CN(CCOc2ccc3c(Nc4ccc(Cl)nc4)nccc3c2)C1] | N-(6-chloropyridin-3-yl)-6-(2- (3-fluoroazetidin-1- yl)ethoxy)isoquinolin-1-amine |
| 117 | 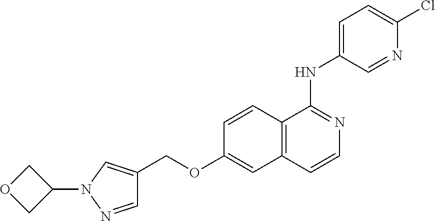 [SMILES: Clc1ccc(Nc2nccc3cc(OCc4cnn(C5COC5)c4)ccc23)cn1] | N-(6-chloropyridin-3-yl)-6-((1- (oxetan-3-yl)-1H-pyrazol-4- yl)methoxy)isoquinolin-1- amine |
| 118 | 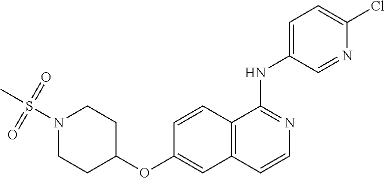 [SMILES: CS(=O)(=O)N1CCC(Oc2ccc3c(Nc4ccc(Cl)nc4)nccc3c2)CC1] | N-(6-chloropyridin-3-yl)-6-((1- (methylsulfonyl)piperidin-4- yl)oxy)isoquinolin-1-amine |
| 119 | 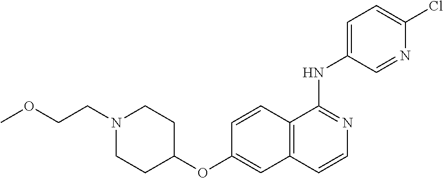 [SMILES: COCCN1CCC(Oc2ccc3c(Nc4ccc(Cl)nc4)nccc3c2)CC1] | N-(6-chloropyridin-3-yl)-6-((1- (2-methoxyethyl)piperidin-4- yl)oxy)isoquinolin-1-amine |
| 120 | 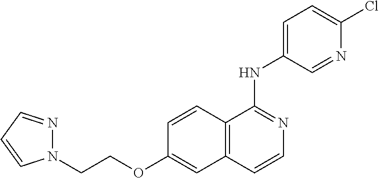 [SMILES: Clc1ccc(Nc2nccc3cc(OCCn4cccn4)ccc23)cn1] | 6-(2-(1H-pyrazol-1-yl)ethoxy)- N-(6-chloropyridin-3- yl)isoquinolin-1-amine |
| 121 | 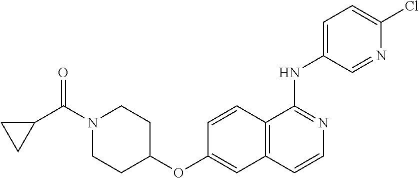 [SMILES: O=C(C1CC1)N1CCC(Oc2ccc3c(Nc4ccc(Cl)nc4)nccc3c2)CC1] | (4-((1-((6-chloropyridin-3- yl)amino)isoquinolin-6- yl)oxy)piperidin-1- yl)(cyclopropyl)methanone |
| 122 | 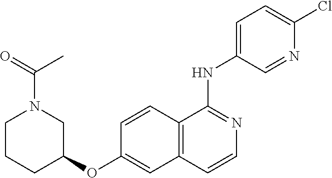 [SMILES: CC(=O)N1CCC[C@H](Oc2ccc3c(Nc4ccc(Cl)nc4)nccc3c2)C1] | (S)-1-(3-((1-((6-chloropyridin- 3-yl)amino)isoquinolin-6- yl)oxy)piperidin-1-yl)ethan-1- one |
| 123 | 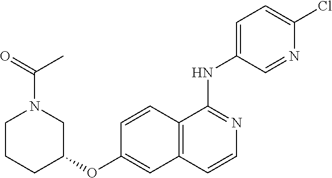 [SMILES: CC(=O)N1CCC[C@@H](Oc2ccc3c(Nc4ccc(Cl)nc4)nccc3c2)C1] | (R)-1-(3-((1-((6-chloropyridin- 3-yl)amino)isoquinolin-6- yl)oxy)piperidin-1-yl)ethan-1- one |
| 124 | 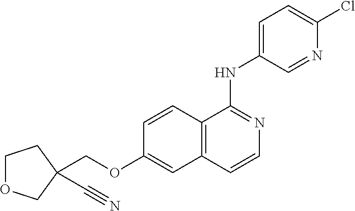 [SMILES: N#CC1(COc2ccc3c(Nc4ccc(Cl)nc4)nccc3c2)CCOC1] | 3-(((1-((6-chloropyridin-3- yl)amino)isoquinolin-6- yl)oxy)methyl)tetrahydrofuran- 3-carbonitrile |
| 125 | 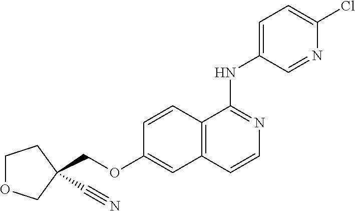 [SMILES: N#C[C@@]1(COc2ccc3c(Nc4ccc(Cl)nc4)nccc3c2)CCOC1] | (R)-3-(((1-((6-chloropyidin-3- yl)amino)isoquinolin-6- yl)oxy)methyl)tetrahydrofuran- 3-carbonitrile |
| 126 | 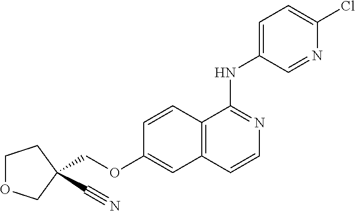 [SMILES: N#C[C@]1(COc2ccc3c(Nc4ccc(Cl)nc4)nccc3c2)CCOC1] | (S)-3-(((1-((6-chloropyridin-3- yl)amino)isoquinolin-6- yl)oxy)methyl)tetrahydrofuran- 3-carbonitrile |
| 127 | 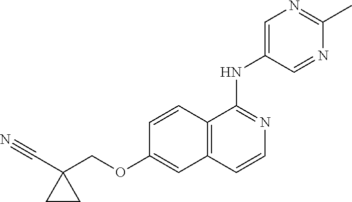 [SMILES: Cc1ncc(Nc2nccc3cc(OCC4(C#N)CC4)ccc23)cn1] | 1-(((1-((2-methylpyrimidin-5- yl)amino)isoquinolin-6- yl)oxy)methyl)cyclopropane- 1-carbonitrile |
| 128 | 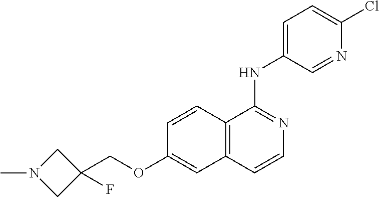 [SMILES: CN1CC(F)(COc2ccc3c(Nc4ccc(Cl)nc4)nccc3c2)C1] | N-(6-chloropyridin-3-yl)-6-((3- fluoro-1-methylazetidin-3- yl)methoxy)isoquinolin-1- amine |
| 129 | 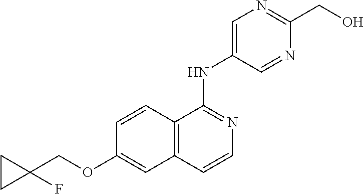 [SMILES: OCc1ncc(Nc2nccc3cc(OCC4(F)CC4)ccc23)cn1] | (5-((6-((1- fluorocyclopropyl)methoxy)iso- quinolin-1-yl)amino)pyrimidin- 2-yl)methanol |
| 130 | 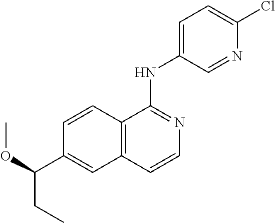 [SMILES: CC[C@@H](OC)c1ccc2c(Nc3ccc(Cl)nc3)nccc2c1] | (R)-N-(6-chloropyridin-3-yl)-6- (1-methoxypropyl)isoquinolin- 1-amine |
| and | | and |
| 131 | 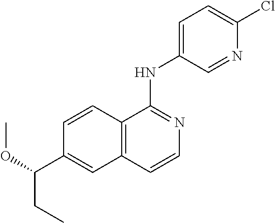 [SMILES: CC[C@H](OC)c1ccc2c(Nc3ccc(Cl)nc3)nccc2c1] | (S)-N-(6-chloropyridin-3-yl)-6- (1-methoxypropyl)isoquinolin- 1-amine |
| 132 | 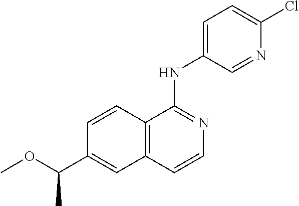 [SMILES: CO[C@H](C)c1ccc2c(Nc3ccc(Cl)nc3)nccc2c1] | (R)-N-(6-chloropyridin-3-yl)-6- (1-methoxyethyl)isoquinolin- 1-amine |
| 133 | 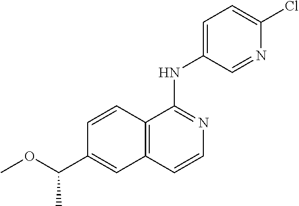 [SMILES: CO[C@@H](C)c1ccc2c(Nc3ccc(Cl)nc3)nccc2c1] | (S)-N-(6-chloropyridin-3-yl)-6- (1-methoxyethyl)isoquinolin- 1-amine |
| 134 | 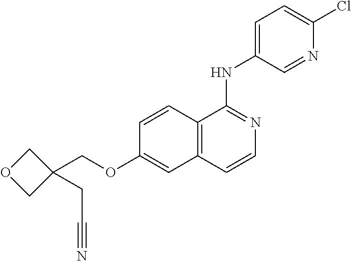 [SMILES: N#CCC1(COc2ccc3c(Nc4ccc(Cl)nc4)nccc3c2)COC1] | 2-(3-(((1-((6-chloropyridin-3- yl)amino)isoquinolin-6- yl)oxy)methyl)oxetan-3- yl)acetonitrile |
| 135 | 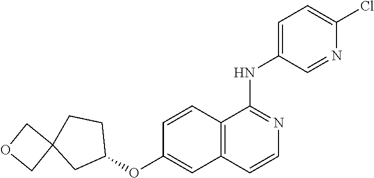 [SMILES: Clc1ccc(Nc2nccc3cc(O[C@H]4CCC5(COC5)C4)ccc23)cn1] | (S)-6-((2-oxaspiro[3.4]octan- 6-yl)oxy)-N-(6-chloropyridin-3- yl)isoquinolin-1-amine |
| 136 | 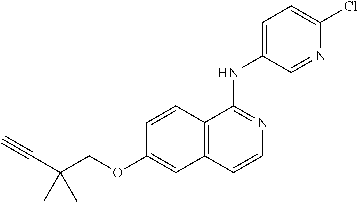 [SMILES: C#CC(C)(C)COc1ccc2c(Nc3ccc(Cl)nc3)nccc2c1] | N-(6-chloropyridin-3-yl)-6- ((2,2-dimethylbut-3-yn-1- yl)oxy)isoquinolin-1-amine |
| 137 | 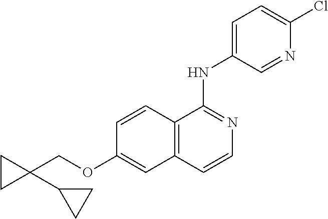 [SMILES: Clc1ccc(Nc2nccc3cc(OCC4(C5CC5)CC4)ccc23)cn1] | 6-([1,1′-bi(cyclopropan)]-1- ylmethoxy)-N-(6- chloropyridin-3-yl)isoquinolin- 1-amine |
| 138 | 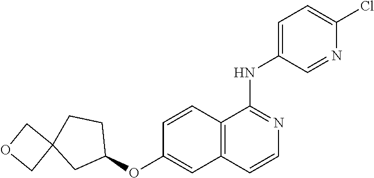 [SMILES: Clc1ccc(Nc2nccc3cc(O[C@@H]4CCC5(COC5)C4)ccc23)cn1] | (R)-6-((2-oxaspiro[3.4]octan- 6-yl)oxy)-N-(6-chloropyridin-3- yl)isoquinolin-1-amine |
| 139 | 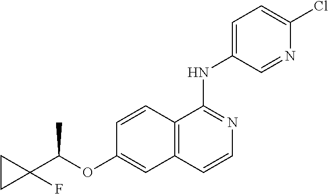 [SMILES: C[C@@H](Oc1ccc2c(Nc3ccc(Cl)nc3)nccc2c1)C1(F)CC1] | (R)-N-(6-chloropyridin-3-yl)-6- (1-(1- fluorocyclopropyl)ethoxy)iso- quinolin-1-amine |
| 140 | 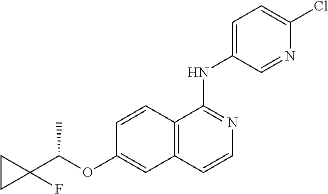 [SMILES: C[C@H](Oc1ccc2c(Nc3ccc(Cl)nc3)nccc2c1)C1(F)CC1] | (S)-N-(6-chloropyridin-3-yl)-6- (1-(1- fluorocyclopropyl)ethoxy)iso- quinolin-1-amine |
| 141 | 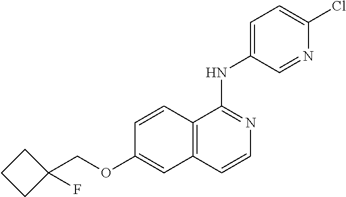 [SMILES: FC1(COc2ccc3c(Nc4ccc(Cl)nc4)nccc3c2)CCC1] | N-(6-chloropyridin-3-yl)-6-((1- fluorocyclobutyl)methoxy)iso- quinolin-1-amine |
| 142 | 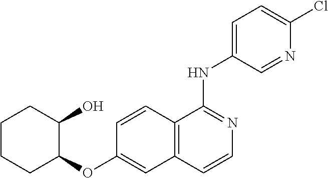 [SMILES: O[C@@H]1CCCC[C@@H]1Oc1ccc2c(Nc3ccc(Cl)nc3)nccc2c1] | cis-2-((1-((6-chloropyridin-3- yl)amino)isoquinolin-6- yl)oxy)cyclohexan-1-ol |
| 143 | 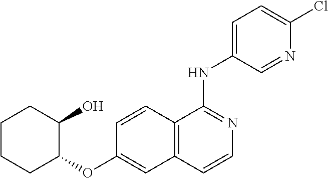 [SMILES: O[C@@H]1CCCC[C@H]1Oc1ccc2c(Nc3ccc(Cl)nc3)nccc2c1] | trans-2-((1-((6-chloropyridin- 3-yl)amino)isoquinolin-6- yl)oxy)cyclohexan-1-ol |
| 144 | 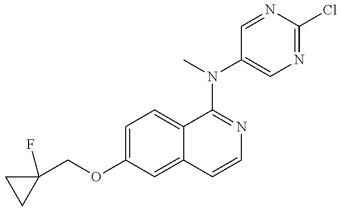 [SMILES: CN(c1cnc(Cl)nc1)c1nccc2cc(OCC3(F)CC3)ccc12] | N-(2-chloropyrimidin-5-yl)-6- ((1- fluorocyclopropyl)methoxy)-N- methylisoquinolin-1-amine |
| 145 | 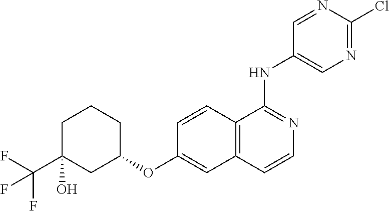 [SMILES: O[C@]1(C(F)(F)F)CCC[C@H](Oc2ccc3c(Nc4cnc(Cl)nc4)nccc3c2)C1] | rac-(1R,3S)-3-((1-((2- chloropyrimidin-5- yl)amino)isoquinolin-6-yl)oxy)- 1-(trifluoromethyl)cyclohexan- 1-ol |
| 146 | 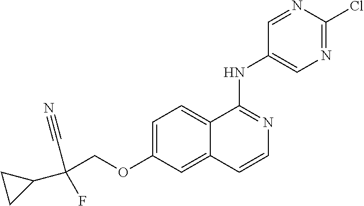 [SMILES: N#CC(F)(COc1ccc2c(Nc3cnc(Cl)nc3)nccc2c1)C1CC1] | 3-((1-((2-chloropyrimidin-5- yl)amino)isoquinolin-6-yl)oxy)- 2-cyclopropyl-2- fluoropropanenitrile |
| 147 | 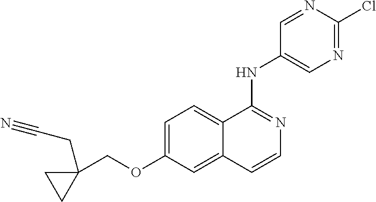 [SMILES: N#CCC1(COc2ccc3c(Nc4cnc(Cl)nc4)nccc3c2)CC1] | 2-(1-(((1-((2-chloropyrimidin- 5-yl)amino)isoquinolin-6- yl)xoy)methyl)cyclopropyl)ace- tonitrile |
| 148 | 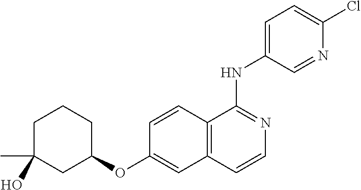 [SMILES: C[C@]1(O)CCC[C@@H](Oc2ccc3c(Nc4ccc(Cl)nc4)nccc3c2)C1] | (1S,3R)-3-((1-((6- chloropyridin-3- yl)amino)isoquinolin-6-yl)oxy)- 1-methylcyclohexan-1-ol |
| 149 | 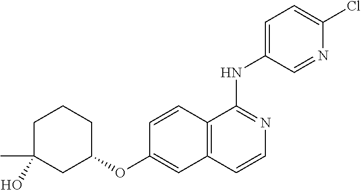 [SMILES: C[C@@]1(O)CCC[C@H](Oc2ccc3c(Nc4ccc(Cl)nc4)nccc3c2)C1] | (1R,3S)-3-((1-((6- chloropyridin-3- yl)amino)isoquinolin-6-yl)oxy)- 1-methylcyclohexan-1-ol |
| 150 | 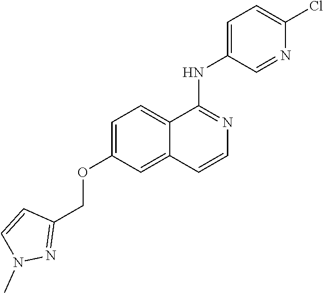 [SMILES: Cn1ccc(COc2ccc3c(Nc4ccc(Cl)nc4)nccc3c2)n1] | N-(6-chloropyridin-3-yl)-6-((1- methyl-1H-pyrazol-3- yl)methoxy)isoquinolin-1- amine |
| 151 | 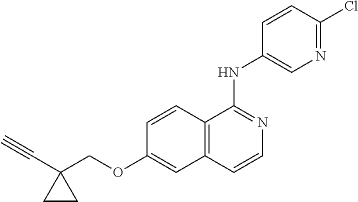 [SMILES: C#CC1(COc2ccc3c(Nc4ccc(Cl)nc4)nccc3c2)CC1] | N-(6-chloropyridin-3-yl)-6-((1- ethynylcyclopropyl)methoxy)iso- quuinolin-1-amine |
| 152 | 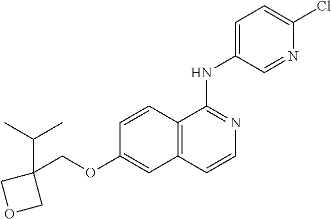 [SMILES: CC(C)C1(COc2ccc3c(Nc4ccc(Cl)nc4)nccc3c2)COC1] | N-(6-chloropyridin-3-yl)-6-((3- isopropyloxetan-3- yl)methoxy)isoquinolin-1- amine |
| 153 | 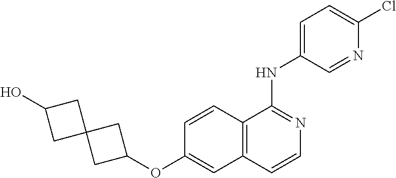 [SMILES: OC1CC2(C1)CC(Oc1ccc3c(Nc4ccc(Cl)nc4)nccc3c1)C2] | 6-((1-((6-chloropyridin-3- yl)amino)isoquinolin-6- yl)oxy)spiro[3.3]heptan-2-ol |
| 154 | 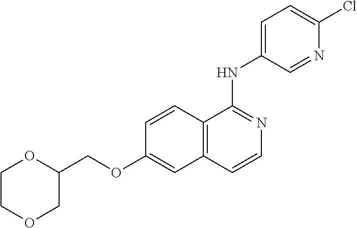 [SMILES: Clc1ccc(Nc2nccc3cc(OCC4COCCO4)ccc23)cn1] | 6-((1,4-dioxan-2-yl)methoxy)- N-(6-chloropyridin-3- yl)isoquinolin-1-amine |
| 155 | 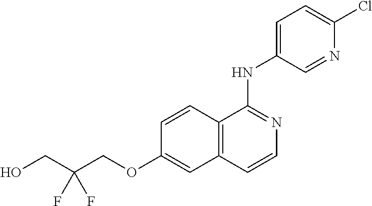 [SMILES: OCC(F)(F)COc1ccc2c(Nc3ccc(Cl)nc3)nccc2c1] | 3-((1-((6-chloropyridin-3- yl)amino)isoquinolin-6-yl)oxy)- 2,2-difluoropropan-1-ol |
| 156 | 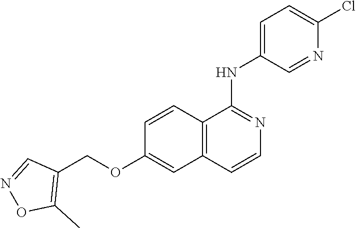 [SMILES: Cc1oncc1COc1ccc2c(Nc3ccc(Cl)nc3)nccc2c1] | N-(6-chloropyridin-3-yl)-6-((5- methylisoxazol-4- yl)methoxy)isoquinolin-1- amine |
| 157 | 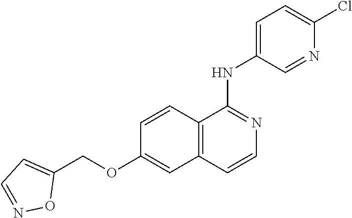 [SMILES: Clc1ccc(Nc2nccc3cc(OCc4ccno4)ccc23)cn1] | N-(6-chloropyridin-3-yl)-6- (isoxazol-5- ylmethoxy)isoquinolin-1- amine |
| 158 | 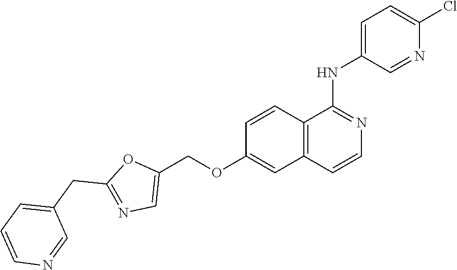 [SMILES: Clc1ccc(Nc2nccc3cc(OCc4cnc(Cc5cccnc5)o4)ccc23)cn1] | N-(6-chloropyridin-3-yl)-6-((2- (pyridin-3-ylmethyl)oxazol-5- yl)methoxy)isoquinolin-1- amine |
| 159 | 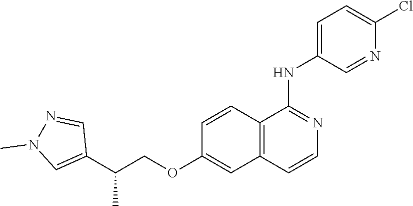 [SMILES: C[C@@H](COc1ccc2c(Nc3ccc(Cl)nc3)nccc2c1)c1cnn(C)c1] | (R)-N-(6-chloropyridin-3-yl)-6- (2-(1-methyl-1H-pyrazol-4- yl)propoxy)isoquinolin-1- amine |
| 160 | 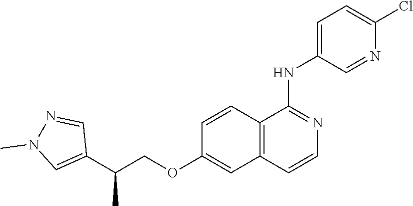 [SMILES: C[C@H](COc1ccc2c(Nc3ccc(Cl)nc3)nccc2c1)c1cnn(C)c1] | (S)-N-(6-chloropyridin-3-yl)-6- (2-(1-methyl-1H-pyrazol-4- yl)propoxy)isoquinolin-1- amine |
| 161 | 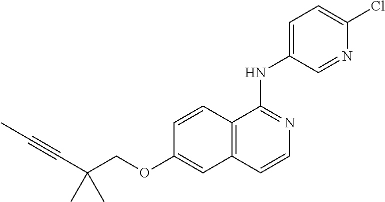 [SMILES: CC#CC(C)(C)COc1ccc2c(Nc3ccc(Cl)nc3)nccc2c1] | N-(6-chloropyridin-3-yl)-6- ((2,2-dimethylpent-3-yn-1- yl)oxy)isoquinolin-1-amine |
| 162 | 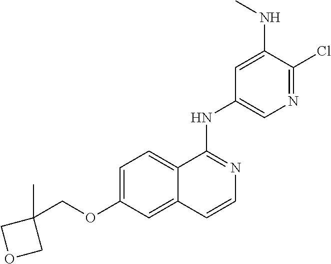 [SMILES: CNc1cc(Nc2nccc3cc(OCC4(C)COC4)ccc23)cnc1Cl] | 2-chloro-N³-methyl-N⁵-(6-((3- methyloxetan-3- yl)methoxy)isoquinolin-1- yl)pyridine-3,5-diamine |
| 163 | 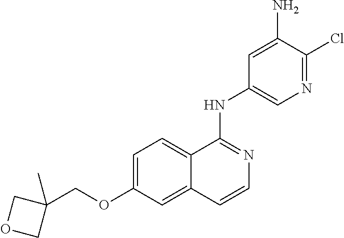 [SMILES: CC1(COc2ccc3c(Nc4cnc(Cl)c(N)c4)nccc3c2)COC1] | 2-chloro-N⁵-(6-((3- methyloxetan-3- yl)methoxy)isoquinolin-1- yl)pyridine-3,5-diamine |
| 164 | 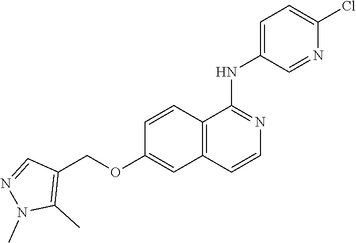 [SMILES: Cc1c(COc2ccc3c(Nc4ccc(Cl)nc4)nccc3c2)cnn1C] | N-(6-chloropyridin-3-yl)-6- ((1,5-dimethyl-1H-pyrazol-4- yl)methoxy)isoquinolin-1- amine |
| 165 | 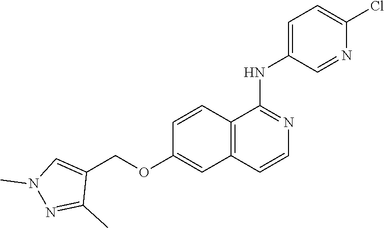 [SMILES: Cc1nn(C)cc1COc1ccc2c(Nc3ccc(Cl)nc3)nccc2c1] | N-(6-chloropyridin-3-yl)-6- ((1,3-dimethyl-1H-pyrazol-4- yl)methoxy)isoquinolin-1- amine |
| 166 | 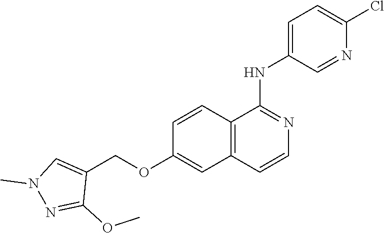 [SMILES: COc1nn(C)cc1COc1ccc2c(Nc3ccc(Cl)nc3)nccc2c1] | N-(6-chloropyridin-3-yl)-6-((3- methoxy-1-methyl-1H- pyrazol-4- yl)methoxy)isoquinolin-1- amine |
| 167 | 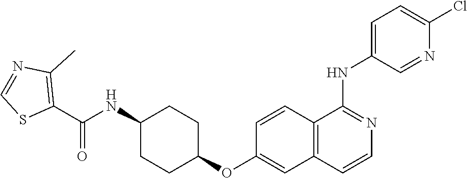 [SMILES: Cc1ncsc1C(=O)N[C@H]1CC[C@@H](Oc2ccc3c(Nc4ccc(Cl)nc4)nccc3c2)CC1] | N-((1s,4s)-4-((1-((6- chloropyridin-3- yl)amino)isoquinolin-6- yl)oxy)cyclohexyl)-4- methylthiazole-5-carboxamide |
| 168 | 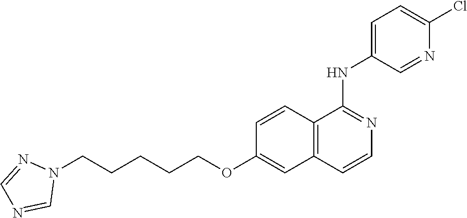 [SMILES: Clc1ccc(Nc2nccc3cc(OCCCCCn4cncn4)ccc23)cn1] | 6-((5-(1H-1,2,4-triazol-1- yl)pentyl)oxy)-N-(6- chloropyridin-3-yl)isoquinolin- 1-amine |
| 169 | 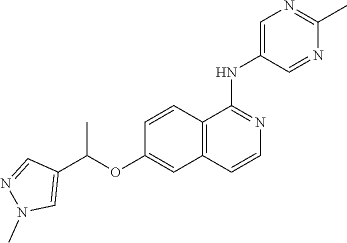 [SMILES: Cc1ncc(Nc2nccc3cc(OC(C)c4cnn(C)c4)ccc23)cn1] | 6-(1-(1-methyl-1H-pyrazol-4- yl)ethoxy)-N-(2- methylpyrimidin-5- yl)isoquinolin-1-amine |
| 170 | 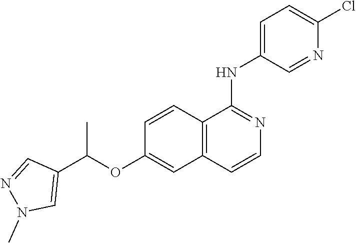 [SMILES: CC(Oc1ccc2c(Nc3ccc(Cl)nc3)nccc2c1)c1cnn(C)c1] | N-(6-chloropyridin-3-yl)-6-(1- (1-methyl-1H-pyrazol-4- yl)ethoxy)isoquinolin-1-amine |
| 171 | 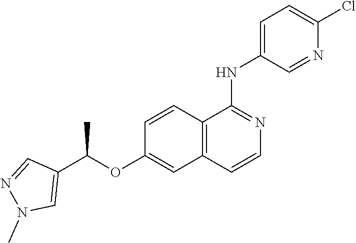 [SMILES: C[C@@H](Oc1ccc2c(Nc3ccc(Cl)nc3)nccc2c1)c1cnn(C)c1] | (R)-N-(6-chloropyridin-3-yl)-6- (1-(1-methyl-1H-pyrazol-4- yl)ethoxy)isoquinolin-1-amine |
| 172 | 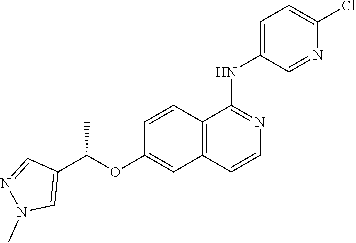 [SMILES: C[C@H](Oc1ccc2c(Nc3ccc(Cl)nc3)nccc2c1)c1cnn(C)c1] | (S)-N-(6-chloropyridin-3-yl)-6- (1-(1-methyl-1H-pyrazol-4- yl)ethoxy)isoquinolin-1-amine |
| 173 | 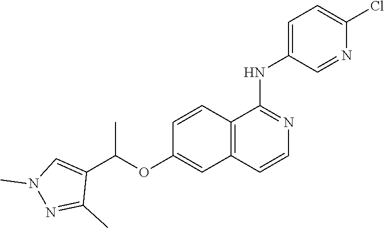 [SMILES: Cc1nn(C)cc1C(C)Oc1ccc2c(Nc3ccc(Cl)nc3)nccc2c1] | N-(6-chloropyridin-3-yl)-6-(1- (1,3-dimethyl-1H-pyrazol-4- yl)ethoxy)isoquinolin-1-amine |
| 174 | 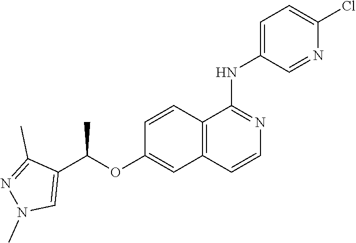 [SMILES: Cc1nn(C)cc1[C@@H](C)Oc1ccc2c(Nc3ccc(Cl)nc3)nccc2c1] | (R)-N-(6-chloropyridin-3-yl)-6- (1-(1,3-dimethyl-1H-pyrazol- 4-yl)ethoxy)isoquinolin-1- amine |
| 175 | 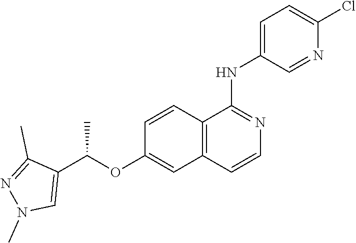 [SMILES: Cc1nn(C)cc1[C@H](C)Oc1ccc2c(Nc3ccc(Cl)nc3)nccc2c1] | (S)-N-(6-chloropyridin-3-yl)-6- (1-(1,3-dimethyl-1H-pyrazol- 4-yl)ethoxy)isoquinolin-1- amine |
| 176 | 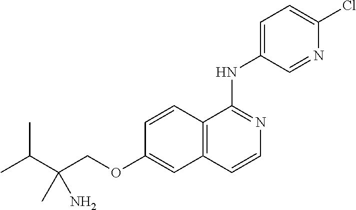 [SMILES: CC(C)C(C)(N)COc1ccc2c(Nc3ccc(Cl)nc3)nccc2c1] | 6-(2-amino-2,3- dimethylbutoxy)-N-(6- chloropryidin-3-yl)isoquinolin- 1-amine |
| 177 | 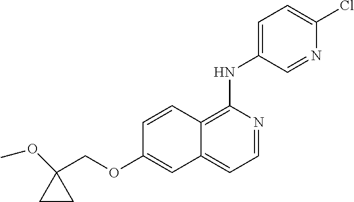 [SMILES: COC1(COc2ccc3c(Nc4ccc(Cl)nc4)nccc3c2)CC1] | N-(6-chloropyridin-3-yl)-6-((1- methoxycyclopropyl)methoxy) isoquinolin-1-amine |
| 178 | 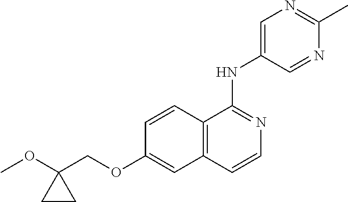 | 6-((1- methoxycyclopropyl)methoxy)- N-(2-methylpyrimidin-5- yl)isoquinolin-1-amine |
| 179 | 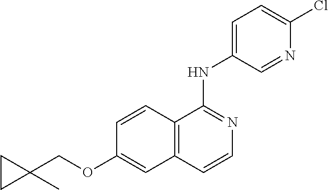 [SMILES: CC1(COc2ccc3c(Nc4ccc(Cl)nc4)nccc3c2)CC1] | N-(6-chloropyridin-3-yl)-6-((1- methylcyclopropyl)methoxy)iso- quinolin-1-amine |
| 180 | 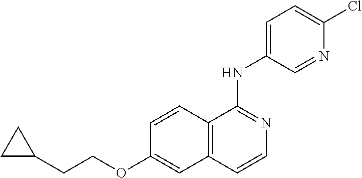 [SMILES: Clc1ccc(Nc2nccc3cc(OCCC4CC4)ccc23)cn1] | N-(6-chloropyridin-3-yl)-6-(2- cyclopropylethoxy)isoquinolin- 1-amine |
| 181 | 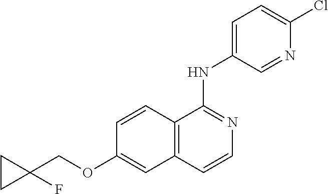 [SMILES: FC1(COc2ccc3c(Nc4ccc(Cl)nc4)nccc3c2)CC1] | N-(6-chloropyridin-3-yl)-6-((1- fluorocyclopropyl)methoxy)iso- quinolin-1-amine |
| 182 | 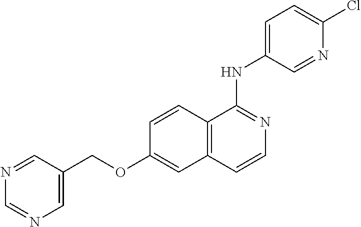 [SMILES: Clc1ccc(Nc2nccc3cc(OCc4cncnc4)ccc23)cn1] | N-(6-chloropyridin-3-yl)-6- (pyrimidin-5- ylmethoxy)isoquinolin-1- amine |
| 183 | 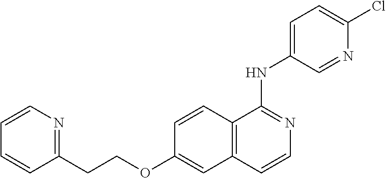 [SMILES: Clc1ccc(Nc2nccc3cc(OCCc4ccccn4)ccc23)cn1] | N-(6-chloropyridin-3-yl)-6-(2- (pyridin-2- yl)ethoxy)isoquinolin-1-amine |
| 184 | 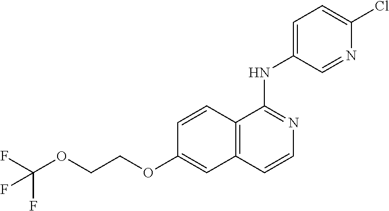 [SMILES: FC(F)(F)OCCOc1ccc2c(Nc3ccc(Cl)nc3)nccc2c1] | N-(6-chloropyridin-3-yl)-6-(2- (trifluoromethoxy)ethoxy)iso- quinolin-1-amine |
| 185 | 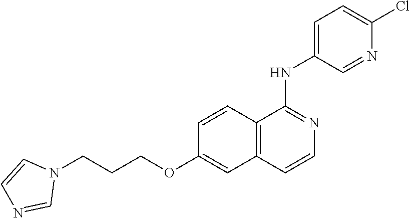 [SMILES: Clc1ccc(Nc2nccc3cc(OCCCn4ccnc4)ccc23)cn1] | 6-(3-(1H-imidazol-1- yl)propoxy)-N-(6- chloropyridin-3-yl)isoquinolin- 1-amine |
| 186 | 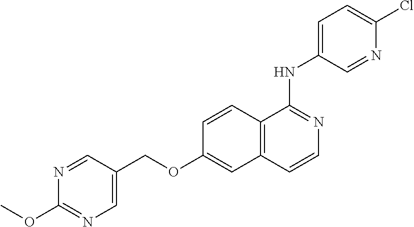 [SMILES: COc1ncc(COc2ccc3c(Nc4ccc(Cl)nc4)nccc3c2)cn1] | N-(6-chloropyridin-3-yl)-6-((2- methoxypyrimidin-5- yl)methoxy)isoquinolin-1- amine |
| 187 | 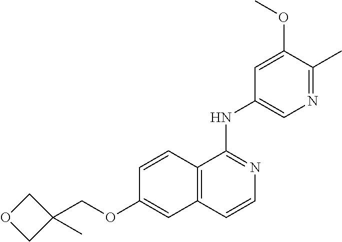 [SMILES: COc1cc(Nc2nccc3cc(OCC4(C)COC4)ccc23)cnc1C] | N-(5-methoxy-6- methylpyridin-3-yl)-6-((3- methyloxetan-3- yl)methoxy)isoquinolin-1- amine |
| 188 | 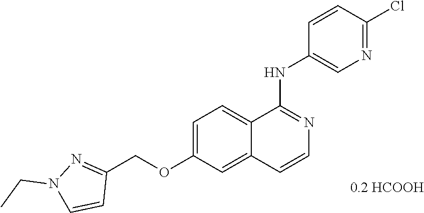 [SMILES: CCn1ccc(COc2ccc3c(Nc4ccc(Cl)nc4)nccc3c2)n1] | N-(6-chloropyridin-3-yl)-6-((1- ehtyl-1H-pyrazol-3- yl)methoxy)isoquinolin-1- amine |
| 189 | 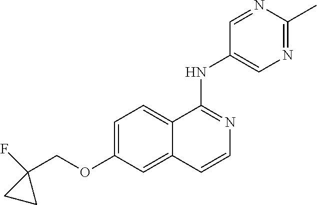 [SMILES: Cc1ncc(Nc2nccc3cc(OCC4(F)CC4)ccc23)cn1] | 6-((1- fluorocyclopropyl)methoxy)-N- (2-methylpyrimidin-5- yl)isoquinolin-1-amine |
| 190 | 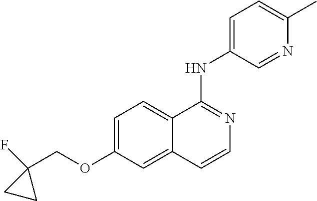 [SMILES: Cc1ccc(Nc2nccc3cc(OCC4(F)CC4)ccc23)cn1] | 6-((1- fluorocyclopropyl)methoxy)-N- (6-methylpyridin-3- yl)isoquinolin-1-amine |
| 191 | 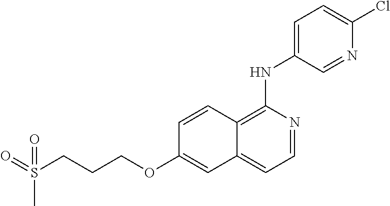 [SMILES: CS(=O)(=O)CCCOc1ccc2c(Nc3ccc(Cl)nc3)nccc2c1] | N-(6-chloropyridin-3-yl)-6-(3- (methylsulfonyl)propoxy)iso- quinolin-1-amine |
| 192 | 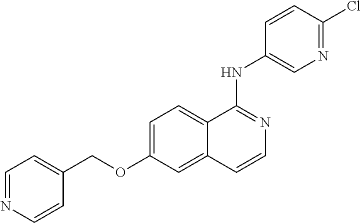 [SMILES: Clc1ccc(Nc2nccc3cc(OCc4ccncc4)ccc23)cn1] | N-(6-chloropyridin-3-yl)-6- (pyridin-4- ylmethoxy)isoquinolin-1- amine |
| 193 | 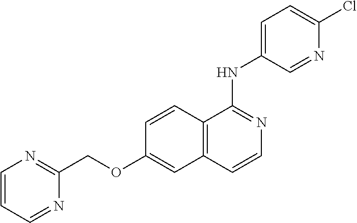 [SMILES: Clc1ccc(Nc2nccc3cc(OCc4ncccn4)ccc23)cn1] | N-(6-chloropyridin-3-yl)-6- (pyrimidin-2- ylmethoxy)isoquinolin-1- amine |
| 194 | 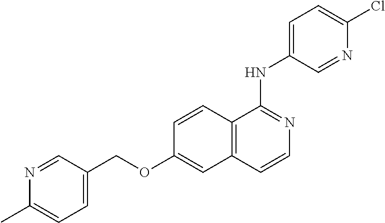 [SMILES: Cc1ccc(COc2ccc3c(Nc4ccc(Cl)nc4)nccc3c2)cn1] | N-(6-chloropyridin-3-yl)-6-((6- methylpyridin-3- yl)methoxy)isoquinolin-1- amine |
| 195 | 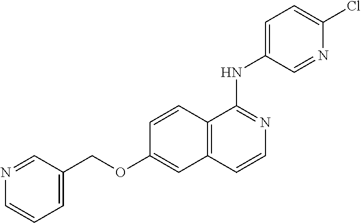 [SMILES: Clc1ccc(Nc2nccc3cc(OCc4cccnc4)ccc23)cn1] | N-(6-chloropyridin-3-yl)-6- (pyridin-3- ylmethoxy)isoquinolin-1- amine |
| 196 | 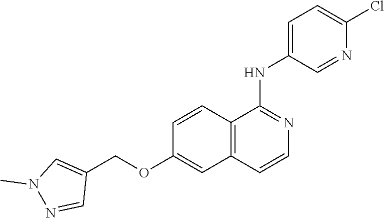 [SMILES: Cn1cc(COc2ccc3c(Nc4ccc(Cl)nc4)nccc3c2)cn1] | N-(6-chloropyridin-3-yl)-6-((1- methyl-1H-pyrazol-4- yl)methoxy)isoquinolin-1- amine |
| 197 | 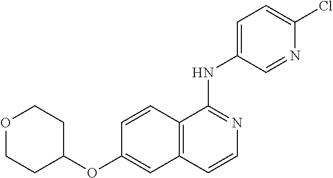 [SMILES: Clc1ccc(Nc2nccc3cc(OC4CCOCC4)ccc23)cn1] | N-(6-chloropyridin-3-yl)-6- ((tetrahydro-2H-pyran-4- yl)oxy)isoquinolin-1-amine |
| 198 | 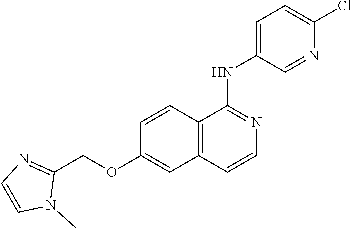 [SMILES: Cn1ccnc1COc1ccc2c(Nc3ccc(Cl)nc3)nccc2c1] | N-(6-chloropyridin-3-yl)-6-((1- methyl-1H-imidazol-2- yl)methoxy)isoquinolin-1- amine |
| 199 | 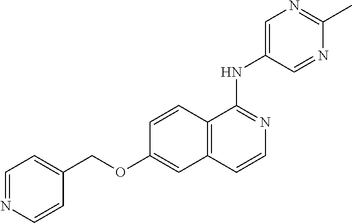 [SMILES: Cc1ncc(Nc2nccc3cc(OCc4ccncc4)ccc23)cn1] | N-(2-methylpyrimidin-5-yl)-6- (pyridin-4- ylmethoxy)isoquinolin-1- amine |
| 200 | 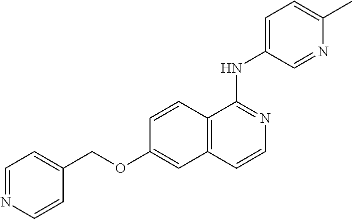 [SMILES: Cc1ccc(Nc2nccc3cc(OCc4ccncc4)ccc23)cn1] | N-(6-methylpyridin-3-yl)-6- (pyridin-4- ylmethoxy)isoquinolin-1- amine |
| 201 | 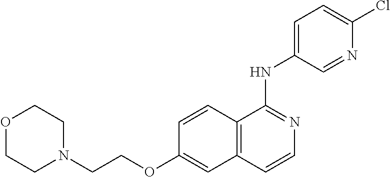 [SMILES: Clc1ccc(Nc2nccc3cc(OCCN4CCOCC4)ccc23)cn1] | N-(6-chloropyridin-3-yl)-6-(2- morpholinoethoxy)isoquinolin- 1-amine |
| 202 | 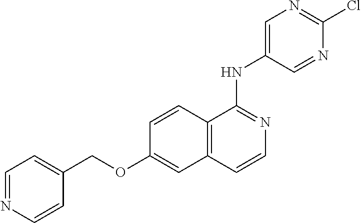 [SMILES: Clc1ncc(Nc2nccc3cc(OCc4ccncc4)ccc23)cn1] | N-(2-chloropyrimidin-5-yl)-6- (pyridin-4- ylmethoxy)isoquinolin-1- amine |
| 203 | 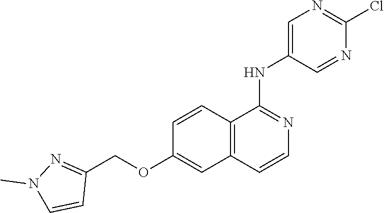 [SMILES: Cn1ccc(COc2ccc3c(Nc4cnc(Cl)nc4)nccc3c2)n1] | N-(2-chloropyrimidin-5-yl)-6- ((1-methyl-1H-pyrazol-3- yl)methoxy)isoquinolin-1- amine |
| 204 | 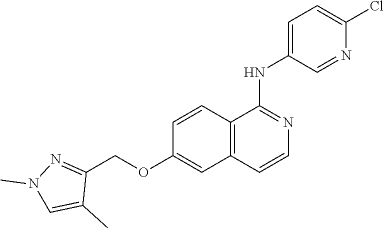 [SMILES: Cc1cn(C)nc1COc1ccc2c(Nc3ccc(Cl)nc3)nccc2c1] | N-(6-chloropyridin-3-yl)-6- ((1,4-dimethyl-1H-pyrazol-3- yl)methoxy)isoquinolin-1- amine |
| 205 | 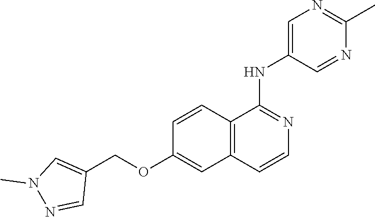 | 6-((1-methyl-1H-pyrazol-4- yl)methoxy)-N-(2- methylpyrimidin-5- yl)isoquinolin-1-amine |
| 206 | 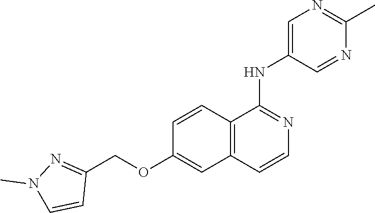 | 6-((1-methyl-1H-pyrazol-3- yl)methoxy)-N-(2- methylpyrimidin-5- yl)isoquinolin-1-amine |
| 207 | 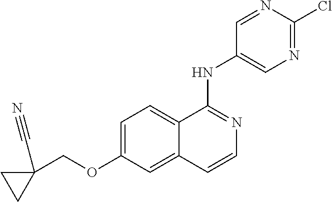 [SMILES: N#CC1(COc2ccc3c(Nc4cnc(Cl)nc4)nccc3c2)CC1] | 1-(((1-((2-chloropyrimidin-5- yl)amino)isoquinolin-6- yl)oxy)methyl)cyclopropane- 1-carbonitrile |
| 208 | 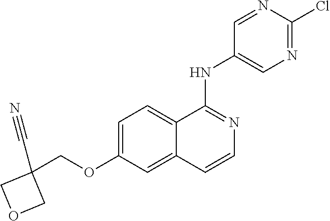 [SMILES: N#CC1(COc2ccc3c(Nc4cnc(Cl)nc4)nccc3c2)COC1] | 3-(((1-((2-chloropyrimidin-5- yl)amino)isoquinolin-6- yl)oxy)methyl)oxetane-3- carbonitrile |
| 209 | 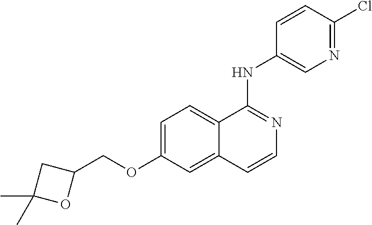 [SMILES: CC1(C)CC(COc2ccc3c(Nc4ccc(Cl)nc4)nccc3c2)O1] | N-(6-chloropyridin-3-yl)-6- ((4,4-dimethyloxetan-2- yl)methoxy)isoquinolin-1- amine |
| 210 | 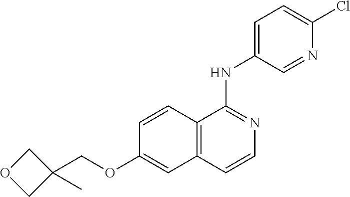 [SMILES: CC1(COc2ccc3c(Nc4ccc(Cl)nc4)nccc3c2)COC1] | N-(6-chloropyridin-3-yl)-6-((3- methyloxetan-3- yl)methoxy)isoquinolin-1- amine |
| 211 | 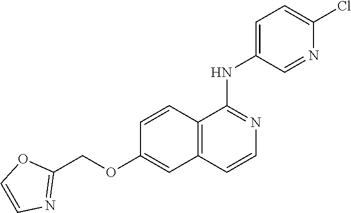 [SMILES: Clc1ccc(Nc2nccc3cc(OCc4ncco4)ccc23)cn1] | N-(6-chloropyridin-3-yl)-6- (oxazol-2- ylmethoxy)isoquinolin-1- amine |
| 212 | 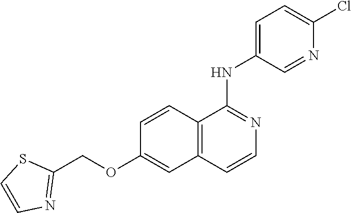 [SMILES: Clc1ccc(Nc2nccc3cc(OCc4nccs4)ccc23)cn1] | N-(6-chloropyridin-3-yl)-6- (thiazol-2- ylmethoxy)isoquinolin-1- amine |
| 213 | 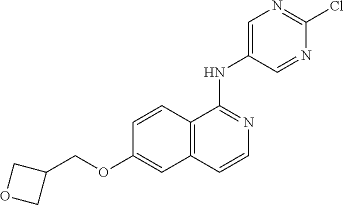 [SMILES: Clc1ncc(Nc2nccc3cc(OCC4COC4)ccc23)cn1] | N-(2-chloropyrimidin-5-yl)-6- (oxetan-3- ylmethoxy)isoquinolin-1- amine |
| 214 | 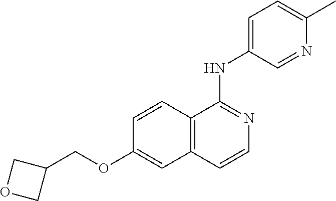 [SMILES: Cc1ccc(Nc2nccc3cc(OCC4COC4)ccc23)cn1] | N-(6-methylpyridin-3-yl)-6- (oxetan-3- ylmethoxy)isoquinolin-1- amine |
| 215 | 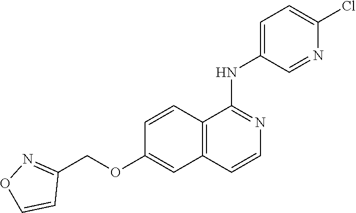 [SMILES: Clc1ccc(Nc2nccc3cc(OCc4ccon4)ccc23)cn1] | N-(6-chloropyridin-3-yl)-6- (isoxazol-3- ylmethoxy)isoquinolin-1- amine |
| 216 | 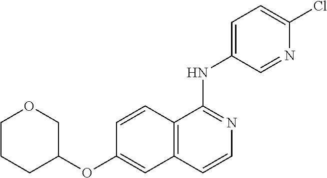 [SMILES: Clc1ccc(Nc2nccc3cc(OC4CCCOC4)ccc23)cn1] | N-(6-chloropyridin-3-yl)-6- ((tetrahydro-2H-pyran-3- yl)oxy)isoquinolin-1-amine |
| 217 | 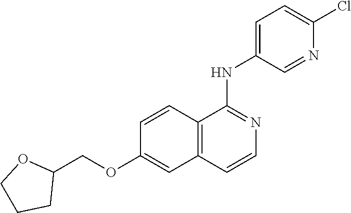 [SMILES: Clc1ccc(Nc2nccc3cc(OCC4CCCO4)ccc23)cn1] | N-(6-chloropyridin-3-yl)-6- ((tetrahydrofuran-2- yl)methoxy)isoquinolin-1- amine |
| 218 | 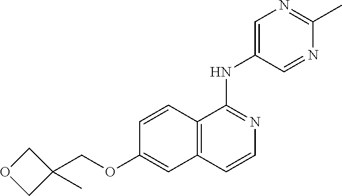 [SMILES: Cc1ncc(Nc2nccc3cc(OCC4(C)COC4)ccc23)cn1] | 6-((3-methyloxetan-3- yl)methoxy)-N-(2- methylpyrimidin-5- yl)isoquinolin-1-amine |
| 219 | 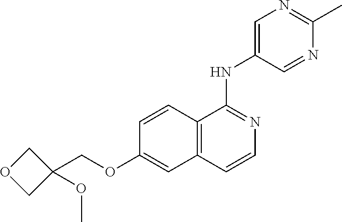 [SMILES: COC1(COc2ccc3c(Nc4cnc(C)nc4)nccc3c2)COC1] | 6-((3-methoxyoxetan-3- yl)methoxy)-N-(2- methylpyrimidin-5- yl)isoquinolin-1-amine |
| 220 | 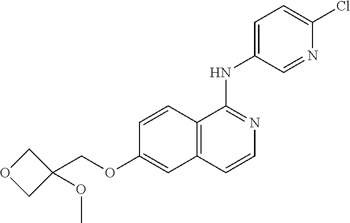 [SMILES: COC1(COc2ccc3c(Nc4ccc(Cl)nc4)nccc3c2)COC1] | N-(6-chloropyridin-3-yl)-6-((3- methoxyoxetan-3- yl)methoxy)isoquinolin-1- amine |
| 221 | 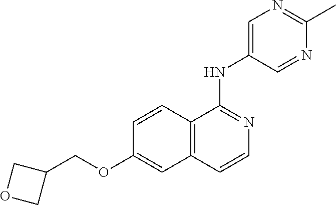 [SMILES: Cc1ncc(Nc2nccc3cc(OCC4COC4)ccc23)cn1] | N-(2-methylpyrimidin-5-yl)-6- (oxetan-3- ylmethoxy)isoquinolin-1- amine |
| 222 | 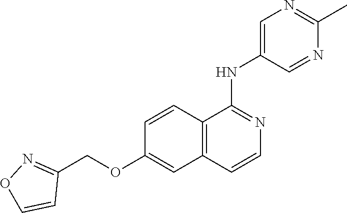 [SMILES: Cc1ncc(Nc2nccc3cc(OCc4ccon4)ccc23)cn1] | 6-(isoxazol-3-ylmethoxy)-N- (2-methylpyrimidin-5- yl)isoquinolin-1-amine |
| 223 | 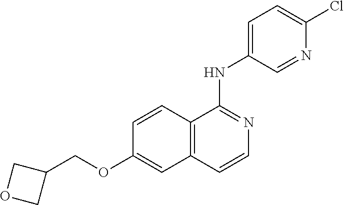 [SMILES: Clc1ccc(Nc2nccc3cc(OCC4COC4)ccc23)cn1] | N-(6-chloropyridin-3-yl)-6- (oxetan-3- ylmethoxy)isoquinolin-1- amine |
| 224 | 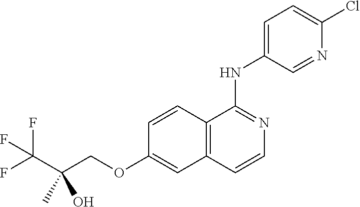 [SMILES: C[C@@](O)(COc1ccc2c(Nc3ccc(Cl)nc3)nccc2c1)C(F)(F)F] | (R)-3-((1-((6-chloropyridin-3- yl)amino)isoquinolin-6-yl)oxy)- 1,1,1-trifluoro-2- methylpropan-2-ol |
| 225 | 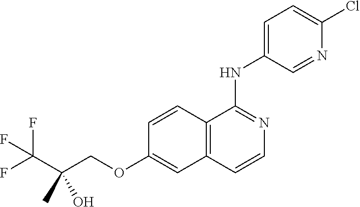 [SMILES: C[C@](O)(COc1ccc2c(Nc3ccc(Cl)nc3)nccc2c1)C(F)(F)F] | (S)-3-((1-((6-chloropyridin-3- yl)amino)isoquinolin-6-yl)oxy)- 1,1,1-trifluoro-2- methylpropan-2-ol |
| 226 | 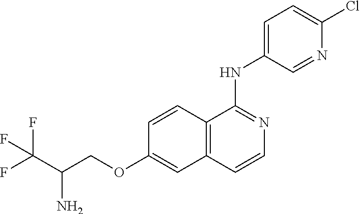 [SMILES: NC(COc1ccc2c(Nc3ccc(Cl)nc3)nccc2c1)C(F)(F)F] | 6-(2-amino-3,3,3- trifluoropropoxy)-N-(6- chloropyridin-3-yl)isoquinolin- 1-amine |
| 227 | 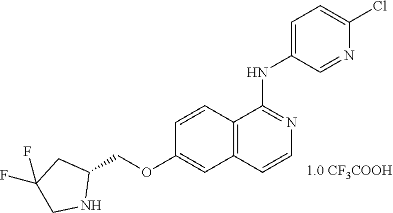 [SMILES: FC1(F)CN[C@@H](COc2ccc3c(Nc4ccc(Cl)nc4)nccc3c2)C1] | (R)-N-(6-chloropyridin-3-yl)-6- ((4,4-difluoropyrrolidin-2- yl)methoxy)isoquinolin-1- amine |
| 228 | 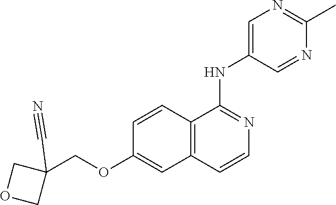 [SMILES: Cc1ncc(Nc2nccc3cc(OCC4(C#N)COC4)ccc23)cn1] | 3-(((1-((2-methylpyrimidin-5- yl)amino)isoquinolin-6- yl)oxy)methyl)oxetane-3- carbonitrile |
| 229 | 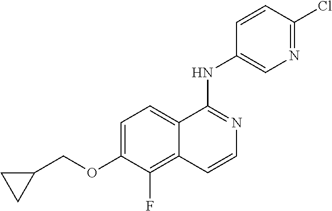 [SMILES: Fc1c(OCC2CC2)ccc2c(Nc3ccc(Cl)nc3)nccc12] | N-(6-chloropyridin-3-yl)-6- (cyclopropylmethoxy)-5- fluoroisoquinolin-1-amine |
| 230 | 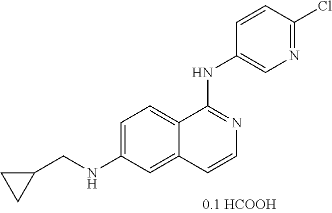 [SMILES: Clc1ccc(Nc2nccc3cc(NCC4CC4)ccc23)cn1] | N¹-(6-chloropyridin-3-yl)-N⁶- (cyclopropylmethyl)isoquinoline- 1,6-diamine formate |
| 231 | 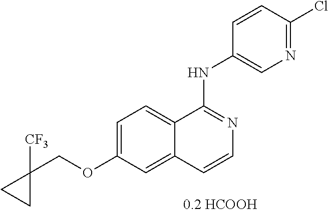 [SMILES: FC(F)(F)C1(COc2ccc3c(Nc4ccc(Cl)nc4)nccc3c2)CC1] | N-(6-chloropyridin-3-yl)-6-((1- (trifluoromethyl)cyclopropyl)meth- oxy)isoquinolin-1-amine formate |
| 232 | 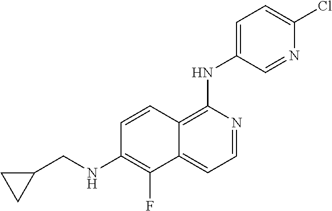 [SMILES: Fc1c(NCC2CC2)ccc2c(Nc3ccc(Cl)nc3)nccc12] | N¹-(6-chloropyridin-3-yl)-N⁶- (cyclopropylmethyl)-5- fluoroisoquinoline-1,6-diamine |
| 233 | 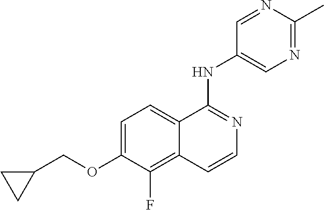 [SMILES: Cc1ncc(Nc2nccc3c(F)c(OCC4CC4)ccc23)cn1] | 6-(cyclopropylmethoxy)-5- fluoro-N-(2-methylpyrimidin-5- yl)isoquinolin-1-amine |
| 234 | 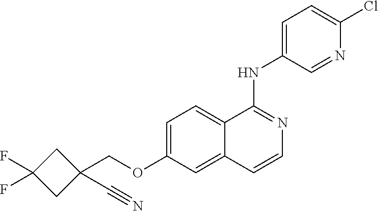 [SMILES: N#CC1(COc2ccc3c(Nc4ccc(Cl)nc4)nccc3c2)CC(F)(F)C1] | 1-(((1-((6-chloropyridin-3- yl)amino)isoquinolin-6- yl)oxy)methyl)-3,3- difluorocylcobutane-1- carbonitrile |
| 235 | 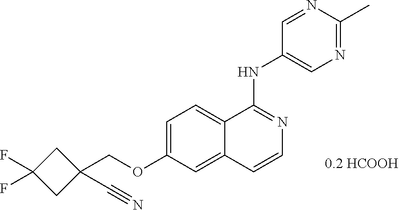 [SMILES: Cc1ncc(Nc2nccc3cc(OCC4(C#N)CC(F)(F)C4)ccc23)cn1] | 3,3-difluoro-1-(((1-((2- methylpyrimidin-5- yl)amino)isoquinolin-6- yl)oxy)methyl)cyclobutane-1- carbonitrile |
| 236 | 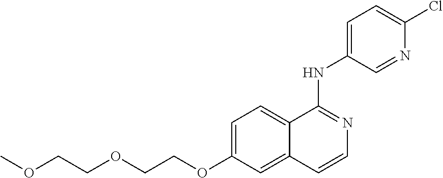 [SMILES: COCCOCCOc1ccc2c(Nc3ccc(Cl)nc3)nccc2c1] | N-(6-chloropyridin-3-yl)-6-(2- (2- methoxyethoxy)ethoxy)iso- quinolin-1-amine |
| 237 | 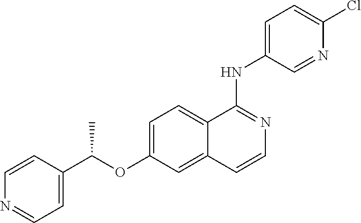 [SMILES: C[C@H](Oc1ccc2c(Nc3ccc(Cl)nc3)nccc2c1)c1ccncc1] | (S)-N-(6-chloropyridin-3-yl)-6- (1-(pyridin-4- yl)ethoxy)isoquinolin-1-amine |
| 238 | 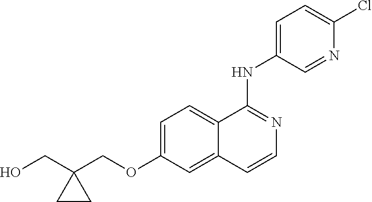 [SMILES: OCC1(COc2ccc3c(Nc4ccc(Cl)nc4)nccc3c2)CC1] | (1-(((1-((6-chloropyridin-3- yl)amino)isoquinolin-6- yl)oxy)methyl)cyclopropyl)meth- anol |
| 239 | 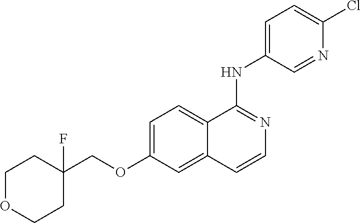 [SMILES: FC1(COc2ccc3c(Nc4ccc(Cl)nc4)nccc3c2)CCOCC1] | N-(6-chloropyridin-3-yl)-6-((4- fluorotetrahydro-2H-pyran-4- yl)methoxy)isoquinolin-1- amine |
| 240 | 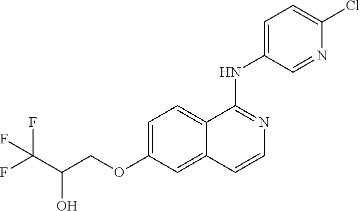 [SMILES: OC(COc1ccc2c(Nc3ccc(Cl)nc3)nccc2c1)C(F)(F)F] | 3-((1-((6-chloropyridin-3- yl)amino)isoquinolin-6-yl)oxy)- 1,1,1-trifluoropropan-2-ol |
| 241 | 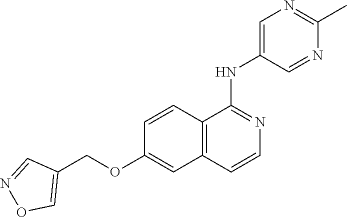 [SMILES: Cc1ncc(Nc2nccc3cc(OCc4cnoc4)ccc23)cn1] | 6-(isoxazol-4-ylmethoxy)-N- (2-methylpyrimidin-5- yl)isoquinolin-1-amine |
| 242 | 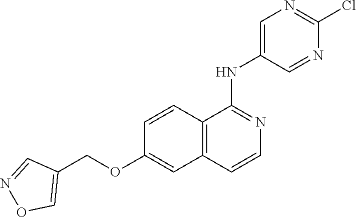 [SMILES: Clc1ncc(Nc2nccc3cc(OCc4cnoc4)ccc23)cn1] | N-(2-chloropyrimidin-5-yl)-6- (isoxazol-4- ylmethoxy)isoquinolin-1- amine |
| 243 | 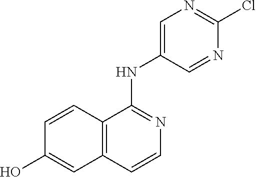 [SMILES: Oc1ccc2c(Nc3cnc(Cl)nc3)nccc2c1] | 1-((2-chloropyrimidin-5- yl)amino)isoquinolin-6-ol |
| 244 | 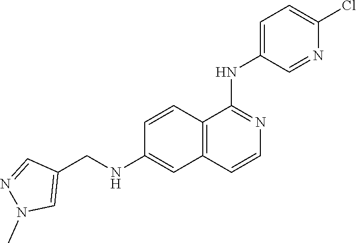 [SMILES: Cn1cc(CNc2ccc3c(Nc4ccc(Cl)nc4)nccc3c2)cn1] | N¹-(6-chloropyridin-3-yl)-N⁶- ((1-methyl-1H-pyrazol-4- yl)methyl)isoquinoline-1,6- diamine |
| 245 | 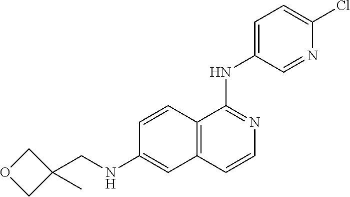 [SMILES: CC1(CNc2ccc3c(Nc4ccc(Cl)nc4)nccc3c2)COC1] | N¹-(6-chloropyridin-3-yl)-N⁶- ((3-methyloxetan-3- yl)methyl)isoquinoline-1,6- diamine |
| 246 | 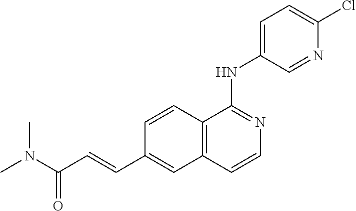 [SMILES: CN(C)C(=O)/C=C/c1ccc2c(Nc3ccc(Cl)nc3)nccc2c1] | (E)-3-(1-((6-chloropyridin-3- yl)amino)isoquinolin-6-yl)- N,N-dimethylacrylamide |
| 247 | 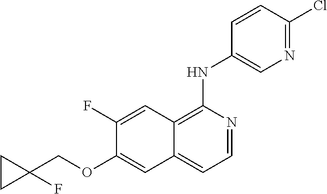 [SMILES: Fc1cc2c(Nc3ccc(Cl)nc3)nccc2cc1OCC1(F)CC1] | N-(6-chloropyridin-3-yl)-7- fluoro-6-((1- fluorocyclopropyl)methoxy)iso- quinolin-1-amine |
| 248 | 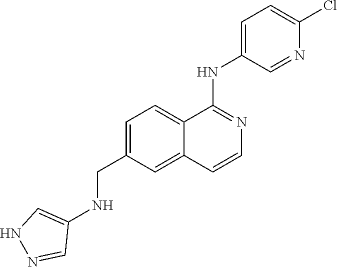 [SMILES: Clc1ccc(Nc2nccc3cc(CNc4cn[nH]c4)ccc23)cn1] | 6-(((1H-pyrazol-4- yl)amino)methyl)-N-(6- chloropyridin-3-yl)isoquinolin- 1-amine |
| 249 | 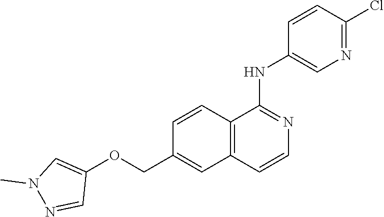 [SMILES: Cn1cc(OCc2ccc3c(Nc4ccc(Cl)nc4)nccc3c2)cn1] | N-(6-chloropyridin-3-yl)-6-(((1- methyl-1H-pyrazol-4- yl)oxy)methyl)isoquinolin-1- amine |
| 250 | 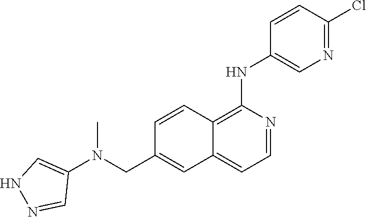 [SMILES: CN(Cc1ccc2c(Nc3ccc(Cl)nc3)nccc2c1)c1cn[nH]c1] | N-(6-chloropyridin-3-yl)-6- ((methyl(1H-pyrazol-4- yl)amino)methyl)isoquinolin-1- amine |
| 251 | 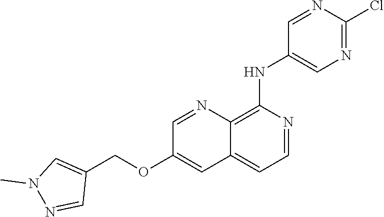 [SMILES: Cn1cc(COc2cnc3c(Nc4cnc(Cl)nc4)nccc3c2)cn1] | N⁶-(6-chloropyridin-3-yl)-N³- ((1-methyl-1H-pyrazol-4- yl)methyl)-1,7-naphthyridine- 3,8-diamine |
| 252 | 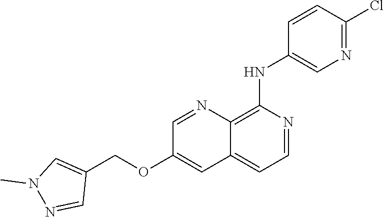 [SMILES: Cn1cc(COc2cnc3c(Nc4ccc(Cl)nc4)nccc3c2)cn1] | N-(6-chloropyridin-3-yl)-3-((1- methyl-1H-pyrazol-4- yl)methoxy)-1,7-naphthyridin- 8-amine |
| 253 | 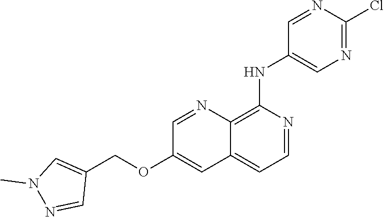 [SMILES: Cn1cc(COc2cnc3c(Nc4cnc(Cl)nc4)nccc3c2)cn1] | N-(2-chloropyrimidin-5-yl)-3- ((1-methyl-1H-pyrazol-4- yl)methoxy)-1,7-naphthyridin- 8-amine |
| 254 | 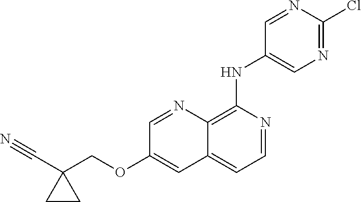 [SMILES: N#CC1(COc2cnc3c(Nc4cnc(Cl)nc4)nccc3c2)CC1] | 1-(((8-((2-chloropyrimidin-5- yl)amino)-1,7-naphthyridin-3- yl)oxy)methyl)cyclopropane- 1-carbonitrile |
| 255 | 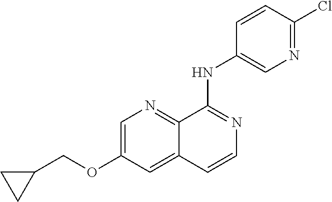 [SMILES: Clc1ccc(Nc2nccc3cc(OCC4CC4)cnc23)cn1] | N-(6-chloropyridin-3-yl)-3- (cyclopropylmethoxy)-1,7- naphthyridin-8-amine |
| 256 | 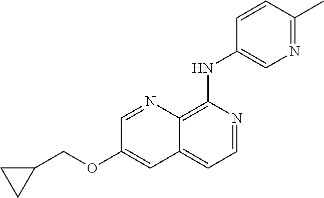 [SMILES: Cc1ccc(Nc2nccc3cc(OCC4CC4)cnc23)cn1] | 3-(cyclopropylmethoxy)-N-(6- methylpyridin-3-yl)-1,7- naphthyridin-8-amine |
| 257 | 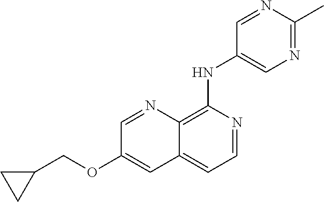 [SMILES: Cc1ncc(Nc2nccc3cc(OCC4CC4)cnc23)cn1] | 3-(cyclopropylmethoxy)-N-(2- methylpyrimidin-5-yl)-1,7- naphthyridin-8-amine |
| 258 | 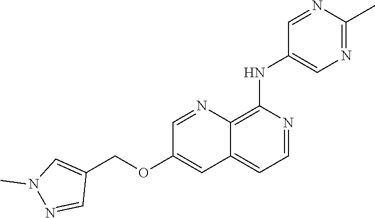 | 3-((1-methyl-1H-pyrazol-4- yl)methoxy)-N-(2- methylpyrimidin-5-yl)-1,7- naphthyridin-8-amine |
| 259 | 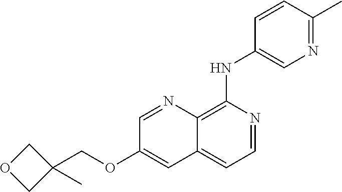 [SMILES: Cc1ccc(Nc2nccc3cc(OCC4(C)COC4)cnc23)cn1] | N-(6-chloropyridin-3-yl)-3-((3- methyloxetan-3-yl)methoxy)- 1,7-naphthyridin-8-amine |
| 260 | 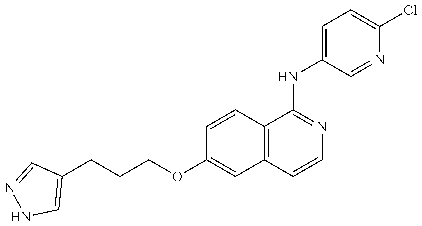 [SMILES: Clc1ccc(Nc2nccc3cc(OCCCc4cn[nH]c4)ccc23)cn1] | 6-(3-(1H-pyrazol-4- yl)propoxy)-N-(6- chloropyridin-3-yl)isoquinolin- 1-amine |
| 261 | 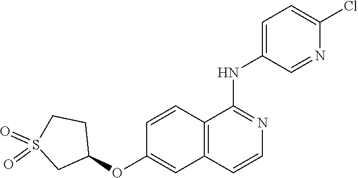 [SMILES: O=S1(=O)CC[C@@H](Oc2ccc3c(Nc4ccc(Cl)nc4)nccc3c2)C1] | (R)-3-((1-((6-chloropyridin-3- yl)amino)isoquinolin-6- yl)oxy)tetrahydrothiophene 1,1-dioxide |
| 262 | 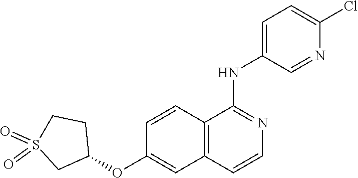 [SMILES: O=S1(=O)CC[C@H](Oc2ccc3c(Nc4ccc(Cl)nc4)nccc3c2)C1] | (S)-3-((1-((6-chloropyridin-3- yl)amino)isoquinolin-6- yl)oxy)tetrahydrothiophene- 1,1-dioxide |
| 263 | 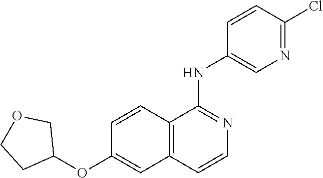 [SMILES: Clc1ccc(Nc2nccc3cc(OC4CCOC4)ccc23)cn1] | N-(6-chloropyridin-3-yl)-6- ((tetrahydrofuran-3- yl)oxy)isoquinolin-1-amine |
| 264 | 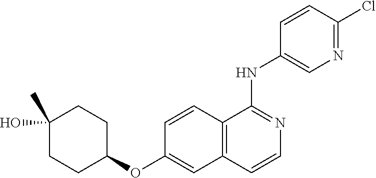 [SMILES: C[C@]1(O)CC[C@H](Oc2ccc3c(Nc4ccc(Cl)nc4)nccc3c2)CC1] | trans-4-((1-((6-chloropyridin- 3-yl)amino)isoquinolin-6- yl)oxy)-1-methylcyclohexan-1- ol |
| 265 | 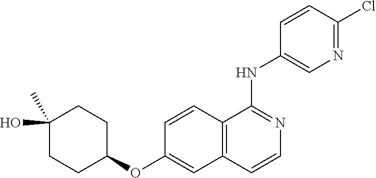 [SMILES: C[C@]1(O)CC[C@@H](Oc2ccc3c(Nc4ccc(Cl)nc4)nccc3c2)CC1] | cis-4-((1-((6-chloropyridin-3- yl)amino)isoquinolin-6-yl)oxy)- 1-methylcyclohexan-1-ol |
| 266 | 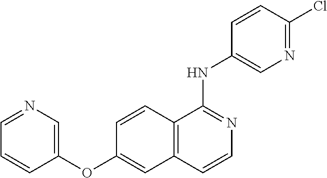 [SMILES: Clc1ccc(Nc2nccc3cc(Oc4cccnc4)ccc23)cn1] | N-(6-chloropyridin-3-yl)-6- (pyridin-3-yloxy)isoquinolin-1- amine |
| 267 | 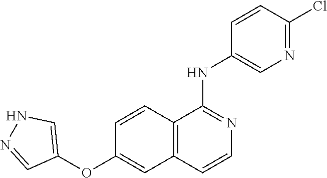 [SMILES: Clc1ccc(Nc2nccc3cc(Oc4cn[nH]c4)ccc23)cn1] | 6-((1H-pyrazol-4-yl)oxy)-N-(6- chloropyridin-3-yl)isoquinolin- 1-amine |
| 268 | 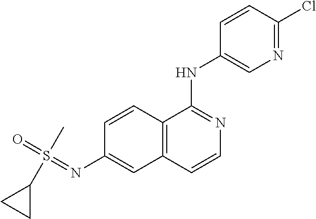 [SMILES: CS(=O)(=Nc1ccc2c(Nc3ccc(Cl)nc3)nccc2c1)C1CC1] | ((1-((6-chloropyridin-3- yl)amino)isoquinolin-6- yl)imino)(cyclopropyl)(methyl)- λ⁶-sulfanone |
| 269 | 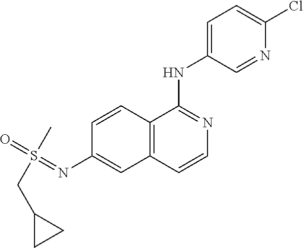 [SMILES: CS(=O)(CC1CC1)=Nc1ccc2c(Nc3ccc(Cl)nc3)nccc2c1] | ((1-((6-chloropyridin-3- yl)amino)isoquinolin-6- yl)imino)(cyclopropylmeth- yl)(methyl)-λ⁶-sulfanone |
| 270 | 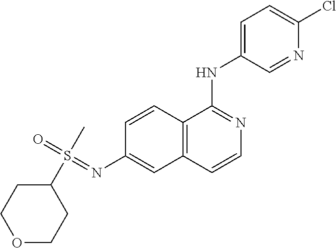 [SMILES: CS(=O)(=Nc1ccc2c(Nc3ccc(Cl)nc3)nccc2c1)C1CCOCC1] | ((1-((6-chloropyridin-3- yl)amino)isoquinolin-6- yl)imino)(methyl)(tetrahydro- 2H-pyran-4-yl)-λ⁶-sulfanone |
| 271 | 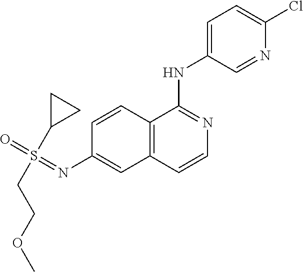 [SMILES: COCCS(=O)(=Nc1ccc2c(Nc3ccc(Cl)nc3)nccc2c1)C1CC1] | ((1-((6-chloropyridin-3- yl)amino)isoquinolin-6- yl)imino)(cyclopropyl)(2- methoxyethyl)-λ⁶-sulfanone |
| 272 | 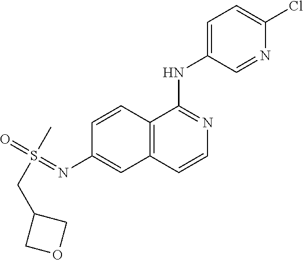 [SMILES: CS(=O)(CC1COC1)=Nc1ccc2c(Nc3ccc(Cl)nc3)nccc2c1] | ((1-((6-chloropyridin-3- yl)amino)isoquinolin-6- yl)imino)(methyl)(oxetan-3- ylmethyl)-λ⁶-sulfanone |
| 273 | 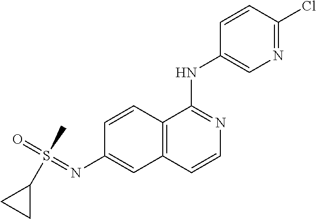 [SMILES: C[S@@](=O)(=Nc1ccc2c(Nc3ccc(Cl)nc3)nccc2c1)C1CC1] | (R)-((1-((6-chloropyridin-3- yl)amino)isoquinolin-6- yl)imino)(cyclopropyl)(methyl)- λ⁶-sulfanone |
| 274 | 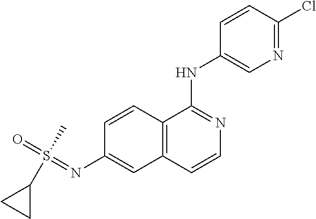 [SMILES: C[S@](=O)(=Nc1ccc2c(Nc3ccc(Cl)nc3)nccc2c1)C1CC1] | (S)-((1-((6-chloropyridin-3- y)amino)isoquinolin-6- yl)imino)(cyclopropyl)(methyl)- λ⁶-sulfanone |
| 275 | 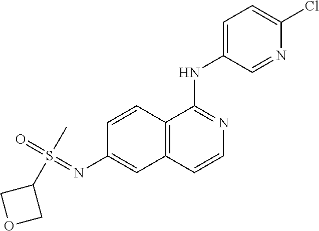 [SMILES: CS(=O)(=Nc1ccc2c(Nc3ccc(Cl)nc3)nccc2c1)C1COC1] | ((1-((6-chloropyridin-3- yl)amino)isoquinolin-6- yl)amino)(methyl)(oxetan-3-yl)- λ⁶-sulfanone |
| 276 | 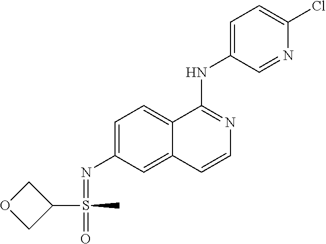 [SMILES: C[S@](=O)(=Nc1ccc2c(Nc3ccc(Cl)nc3)nccc2c1)C1COC1] | (S)-((1-((6-chloropyridin-3- yl)amino)isoquinolin-6- yl)imino)(methyl)(oxetan-3-yl)- λ⁶-sulfanone) |
| 277 | 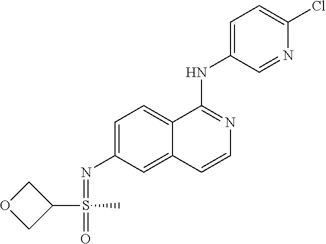 [SMILES: C[S@@](=O)(=Nc1ccc2c(Nc3ccc(Cl)nc3)nccc2c1)C1COC1] | (R)-((1-((6-chloropyridin-3- yl)amino)isoquinolin-6- yl)imino)(methyl)(oxetan-3-yl)- λ⁶-sulfanone) |
| 278 | 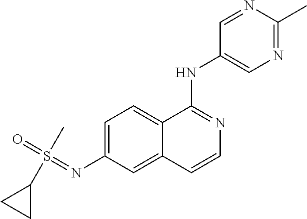 [SMILES: Cc1ncc(Nc2nccc3cc(N=S(C)(=O)C4CC4)ccc23)cn1] | cyclopropyl(methyl)((1-((2- methylpyrimidin-5- yl)amino)isoquinolin-6- yl)imino)-λ⁶-sulfanone |
| 279 | 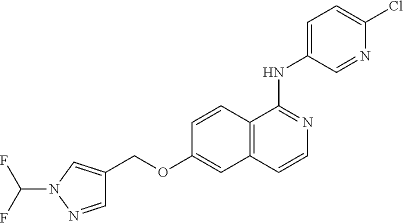 [SMILES: FC(F)n1cc(COc2ccc3c(Nc4ccc(Cl)nc4)nccc3c2)cn1] | N-(6-chloropyridin-3-yl)-6-((1- (difluoromethyl)-1H-pyrazol-4- yl)methoxy)isoquinolin-1- amine |
| 280 | 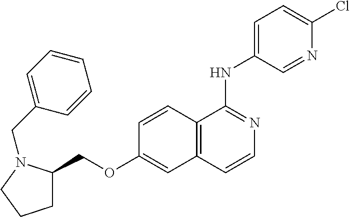 [SMILES: Clc1ccc(Nc2nccc3cc(OC[C@H]4CCCN4Cc4ccccc4)ccc23)cn1] | (R)-6-((1-benzylpyrrolidin-2- y)methoxy)-N-(6- chloropyridin-3-yl)isoquinolin- 1-amine |
| 281 | 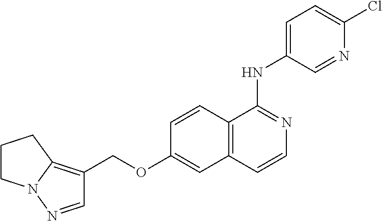 [SMILES: Clc1ccc(Nc2nccc3cc(OCc4cnn5c4CCC5)ccc23)cn1] | N-(6-chloropyridin-3-yl)-6- ((5,6-dihydro-4H-pyrrolo[1,2- b]pyrazol-3- y)methoxy)isoquinolin-1- amine |
| 282 | 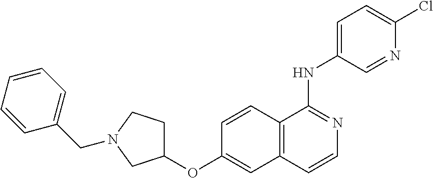 [SMILES: Clc1ccc(Nc2nccc3cc(OC4CCN(Cc5ccccc5)C4)ccc23)cn1] | 6-((1-benzylpyrrolidin-3- yl)oxy)-N-(6-chloropyridin-3- yl)isoquinolin-1-amine |
| 283 | 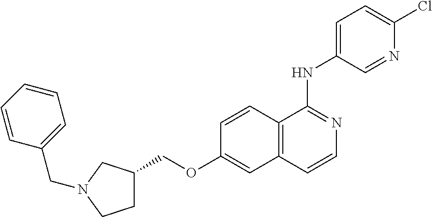 [SMILES: Clc1ccc(Nc2nccc3cc(OC[C@@H]4CCN(Cc5ccccc5)C4)ccc23)cn1] | (R)-6-((1-benzylpyrrolidin-3- yl)methoxy)-N-(6- chloropyridin-3-yl)isoquinolin- 1-amine |
| 284 | 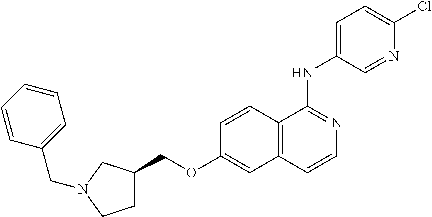 [SMILES: Clc1ccc(Nc2nccc3cc(OC[C@H]4CCN(Cc5ccccc5)C4)ccc23)cn1] | (S)-6-((1-benzylpyrrolidin-3- yl)methoxy)-N-(6- chloropyridin-3-yl)isoquinolin- 1-amine |
| 285 | 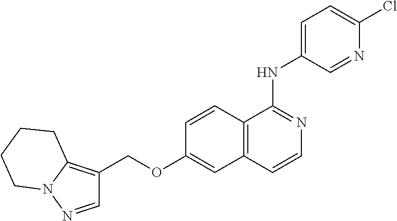 [SMILES: Clc1ccc(Nc2nccc3cc(OCc4cnn5c4CCCC5)ccc23)cn1] | N-(6-chloropyridin-3-yl)-6- ((4,5,6,7- tetrahydropyrazolo[1,5- a]pyridin-3- yl)methoxy)isoquinolin-1- amine |
| 286 | 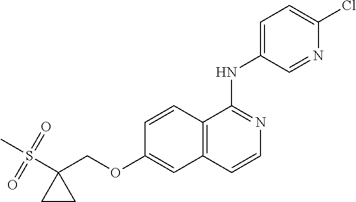 [SMILES: CS(=O)(=O)C1(COc2ccc3c(Nc4ccc(Cl)nc4)nccc3c2)CC1] | N-(6-chloropyridin-3-yl)-6-((1- (methylsulfonyl)cyclopropyl)meth- oxy)isoquinolin-1-amine |
| 287 | 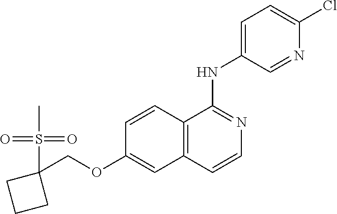 [SMILES: CS(=O)(=O)C1(COc2ccc3c(Nc4ccc(Cl)nc4)nccc3c2)CCC1] | N-(6-chloropyridin-3-yl)-6-((1- (methylsulfonyl)cyclobutyl)meth- oxy)isoquinolin-1-amine |
| 288 | 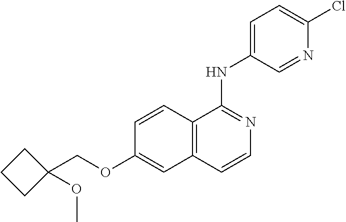 [SMILES: COC1(COc2ccc3c(Nc4ccc(Cl)nc4)nccc3c2)CCC1] | N-(6-chloropyridin-3-yl)-6-((1- methoxycyclobutyl)methoxy)iso- quinolin-1-amine |
| 289 | 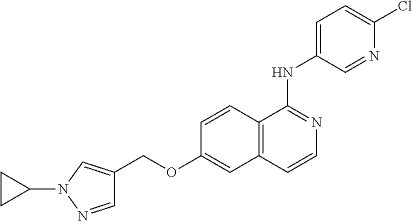 [SMILES: Clc1ccc(Nc2nccc3cc(OCc4cnn(C5CC5)c4)ccc23)cn1] | N-(6-chloropyridin-3-yl)-6-((1- cyclopropyl-1H-pyrazol-4- yl)methoxy)isoquinolin-1- amine |
| 290 | 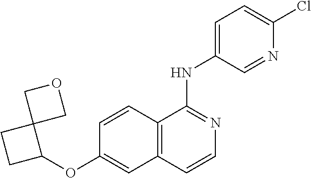 [SMILES: Clc1ccc(Nc2nccc3cc(OC4CCC45COC5)ccc23)cn1] | 6-((1-oxaspiro[3.3]heptan-5- yl)oxy)-N-(6-chloropyridin-3- yl)isoquinolin-1-amine |
| 291 | 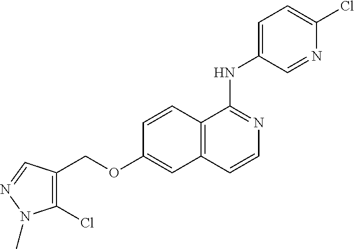 [SMILES: Cn1ncc(COc2ccc3c(Nc4ccc(Cl)nc4)nccc3c2)c1Cl] | 6-((5-chloro-1-methyl-1H- pyrazol-4-yl)methoxy)-N-(6- chloropyridin-3-yl)isoquinolin- 1-amine |
| 292 | 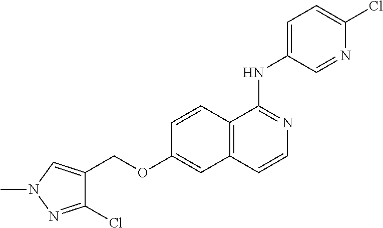 [SMILES: Cn1cc(COc2ccc3c(Nc4ccc(Cl)nc4)nccc3c2)c(Cl)n1] | 6-((3-chloro-1-methyl-1H- pyrazol-4-yl)methoxy)-N-(6- chloropyridin-3-yl)isoquinolin- 1-amine |
| 293 | 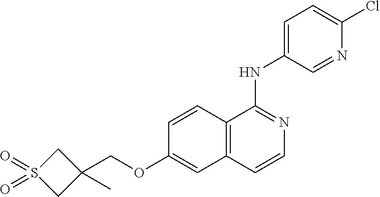 [SMILES: CC1(COc2ccc3c(Nc4ccc(Cl)nc4)nccc3c2)CS(=O)(=O)C1] | 3-(((1-((6-chloropyridin-3- yl)amino)isoquinolin-6- yl)oxy)methyl)-3- methylthietane 1,1-dioxide |
| 294 | 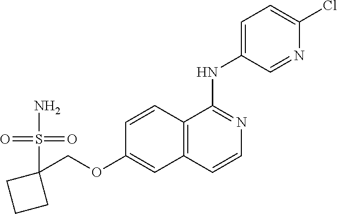 [SMILES: NS(=O)(=O)C1(COc2ccc3c(Nc4ccc(Cl)nc4)nccc3c2)CCC1] | 1-(((1-((6-chloropyridin-3- yl)amino)isoquinolin-6- yl)oxy)methyl)cyclobutane-1- sulfonamide |
| 295 | 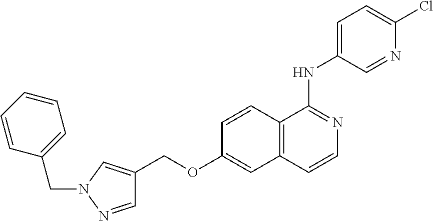 [SMILES: Clc1ccc(Nc2nccc3cc(OCc4cnn(Cc5ccccc5)c4)ccc23)cn1] | 6-((1-benzyl-1H-pyrazol-4- yl)methoxy)-N-(6- chloropyridin-3-yl)isoquinolin- 1-amine |
| 296 | 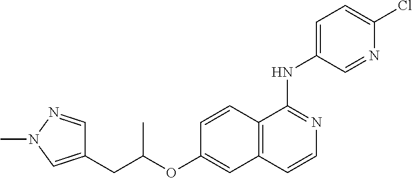 [SMILES: CC(Cc1cnn(C)c1)Oc1ccc2c(Nc3ccc(Cl)nc3)nccc2c1] | N-(6-chloropyridin-3-yl)-6-((1- (1-methyl-1H-pyrazol-4- yl)propan-2- yl)oxy)isoquinolin-1-amine |
| 297 | 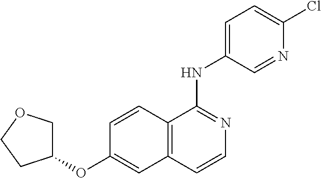 [SMILES: Clc1ccc(Nc2nccc3cc(O[C@@H]4CCOC4)ccc23)cn1] | (R)-N-(6-chloropyridin-3-yl)-6- ((tetrahydrofuran-3- yl)oxy)isoquinolin-1-amine |
| 298 | 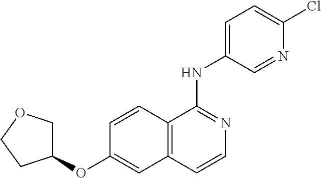 [SMILES: Clc1ccc(Nc2nccc3cc(O[C@H]4CCOC4)ccc23)cn1] | (S)-N-(6-chloropyridin-3-yl)-6- ((tetrahydrofuran-3- yl)oxy)isoquinolin-1-amine |
| 299 | 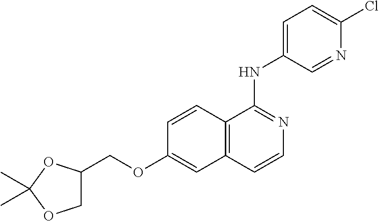 [SMILES: CC1(C)OCC(COc2ccc3c(Nc4ccc(Cl)nc4)nccc3c2)O1] | N-(6-chloropyridin-3-yl)-6- ((2,2-dimethyl-1,3-dioxolan-4- yl)methoxy)isoquinolin-1- amine |
| 300 | 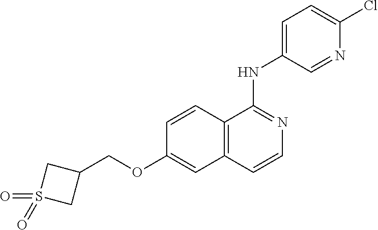 [SMILES: O=S1(=O)CC(COc2ccc3c(Nc4ccc(Cl)nc4)nccc3c2)C1] | 3-(((1-((6-chloropyridin-3- yl)amino)isoquinolin-6- yl)oxy)methyl)thietane 1,1- dioxide |
| 301 | 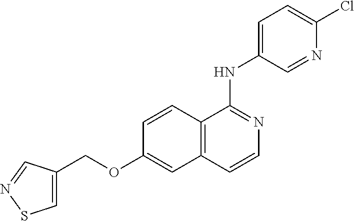 [SMILES: Clc1ccc(Nc2nccc3cc(OCc4cnsc4)ccc23)cn1] | N-(6-chloropyridin-3-yl)-6- (isothiazol-4- ylmethoxy)isoquinolin-1- amine |
| 302 | 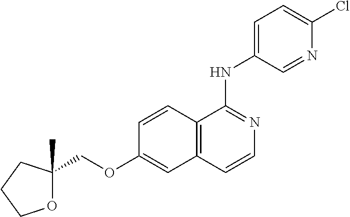 [SMILES: C[C@]1(COc2ccc3c(Nc4ccc(Cl)nc4)nccc3c2)CCCO1] | (R)-N-(6-chloropyridin-3-yl)-6- ((2-methyltetrahydrofuran-2- yl)methoxy)isoquinolin-1- amine |
| 303 | 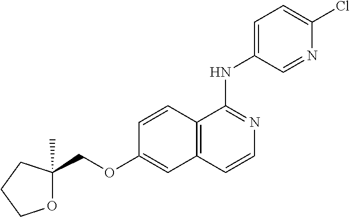 [SMILES: C[C@@]1(COc2ccc3c(Nc4ccc(Cl)nc4)nccc3c2)CCCO1] | (S)-N-(6-chloropyridin-3-yl)-6- ((2-methyltetrahydrofuran-2- yl)methoxy)isoquinolin-1- amine |
| 304 | 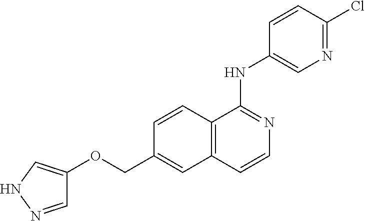 [SMILES: Clc1ccc(Nc2nccc3cc(COc4cn[nH]c4)ccc23)cn1] | 6-(((1H-pyrazol-4- yl)oxy)methyl)-N-(6- chloropyridin-3-yl)isoquinolin- 1-amine |
| 305 | 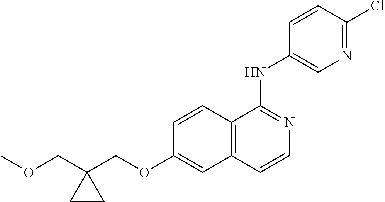 [SMILES: COCC1(COc2ccc3c(Nc4ccc(Cl)nc4)nccc3c2)CC1] | 1-(4-((1-((6-chloropyridin-3- yl)amino)isoquinolin-6- yl)oxy)piperidin-1-yl)ethan-1- one |
| 306 | 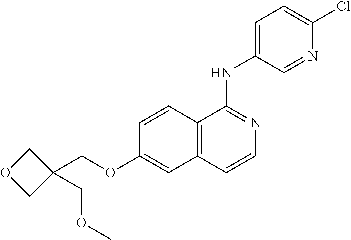 [SMILES: COCC1(COc2ccc3c(Nc4ccc(Cl)nc4)nccc3c2)COC1] | N-(6-chloropyridin-3-yl)-6-((3- (methoxymethyl)oxetan-3- yl)methoxy)isoquinolin-1- amine |
| 307 | 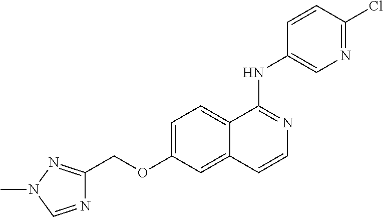 [SMILES: Cn1cnc(COc2ccc3c(Nc4ccc(Cl)nc4)nccc3c2)n1] | N-(6-chloropyridin-3-yl)-6-((1- methyl-1H-1,2,4-triazol-3- yl)methoxy)isoquinolin-1- amine |
| 308 | 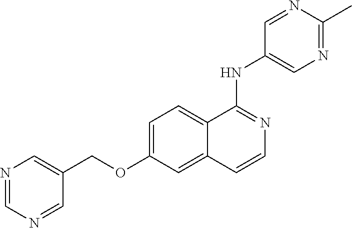 [SMILES: Cc1ncc(Nc2nccc3cc(OCc4cncnc4)ccc23)cn1] | N-(2-methylpyrimidin-5-yl)-6- (pyrimidin-5- ylmethoxy)isoquinolin-1- amine |
| 309 | 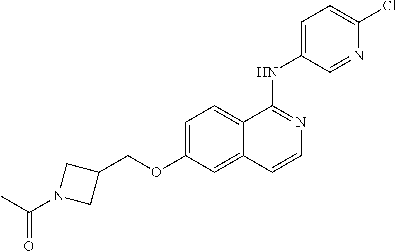 [SMILES: CC(=O)N1CC(COc2ccc3c(Nc4ccc(Cl)nc4)nccc3c2)C1] | 1-(3-(((1-((6-chloropyridin-3- yl)amino)isoquinolin-6- yl)oxy)methyl)azetidin-1- yl)ethan-1-one |
| 310 | 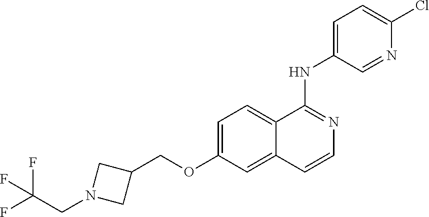 [SMILES: FC(F)(F)CN1CC(COc2ccc3c(Nc4ccc(Cl)nc4)nccc3c2)C1] | N-(6-chloropyridin-3-yl)-6-((1- (2,2,2-trifluoroethyl)azetidin-3- y)methoxy)isoquinolin-1- amine |
| 311 | 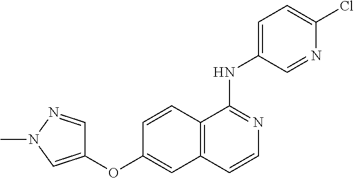 [SMILES: Cn1cc(Oc2ccc3c(Nc4ccc(Cl)nc4)nccc3c2)cn1] | N-(6-chloropyridin-3-yl)-6-((1- methyl-1H-pyrazol-4- yl)oxy)isoquinolin-1-amine |
| 312 | 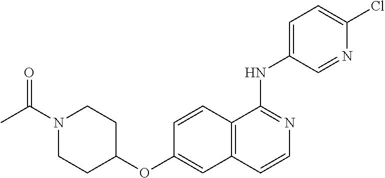 [SMILES: CC(=O)N1CCC(Oc2ccc3c(Nc4ccc(Cl)nc4)nccc3c2)CC1] | 1-(4-((1-((6-chloropyridin-3- yl)amino)isoquinolin-6- yl)oxy)piperidin-1-yl)ethan-1- one |
| 313 | 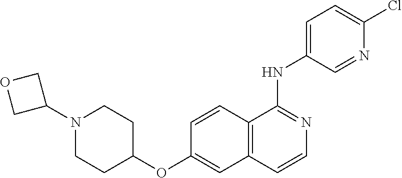 [SMILES: Clc1ccc(Nc2nccc3cc(OC4CCN(C5COC5)CC4)ccc23)cn1] | N-(6-chloropyridin-3-yl)-6-((1- (oxetan-3-yl)piperidin-4- yl)oxy)isoquinolin-1-amine |
| 314 | 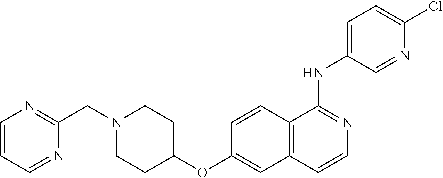 [SMILES: Clc1ccc(Nc2nccc3cc(OC4CCN(Cc5ncccn5)CC4)ccc23)cn1] | N-(6-chloropyridin-3-yl)-6-((1- (pyrimidin-2- ylmethyl)piperidin-4- yl)oxy)isoquinolin-1-amine |
| 315 | 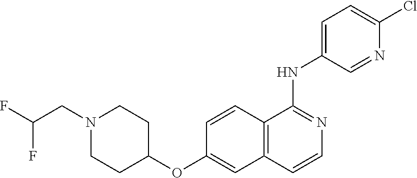 [SMILES: FC(F)CN1CCC(Oc2ccc3c(Nc4ccc(Cl)nc4)nccc3c2)CC1] | N-(6-chloropyridin-3-yl)-6-((1- (2,2-difluoroethyl)piperidin-4- yl)oxy)isoquinolin-1-amine |
| 316 | 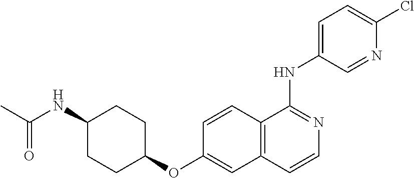 [SMILES: CC(=O)N[C@H]1CC[C@@H](Oc2ccc3c(Nc4ccc(Cl)nc4)nccc3c2)CC1] | N-(cis-4-((1-((6-chloropyridin- 3-yl)amino)isoquinolin-6- yl)oxy)cyclohexyl)acetamide |
| 317 | 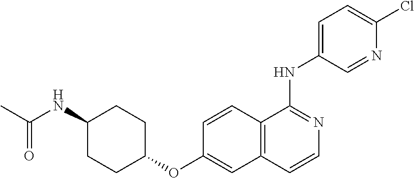 [SMILES: CC(=O)N[C@H]1CC[C@H](Oc2ccc3c(Nc4ccc(Cl)nc4)nccc3c2)CC1] | N-(trans-4-((1-((6- chloropyridin-3- yl)amino)isoquinolin-6- yl)oxy)cyclohexyl)acetamide |
| 318 | 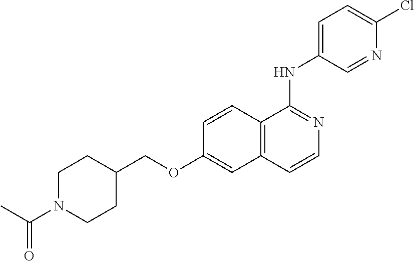 [SMILES: CC(=O)N1CCC(COc2ccc3c(Nc4ccc(Cl)nc4)nccc3c2)CC1] | 1-(4-(((1-((6-chloropyridin-3- yl)amino)isoquinolin-6- yl)oxy)methyl)piperidin-1- yl)ethan-1-one |
| 319 | 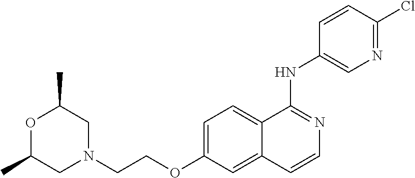 [SMILES: C[C@@H]1CN(CCOc2ccc3c(Nc4ccc(Cl)nc4)nccc3c2)C[C@H](C)O1] | N-(6-chloropyridin-3-yl)-6-(2- ((2S,6R)-2,6- dimethylmorpholino)ethoxy)iso- quinolin-1-amine |
| 320 | 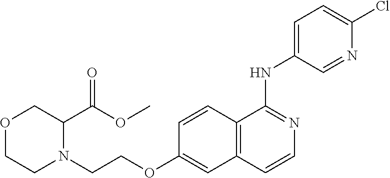 [SMILES: COC(=O)C1COCCN1CCOc1ccc2c(Nc3ccc(Cl)nc3)nccc2c1] | methyl 4-(2-((1-((6- chloropyridin-3- yl)amino)isoquinolin-6- yl)oxy)ethyl)morpholine-3- carboxylate |
| 321 | 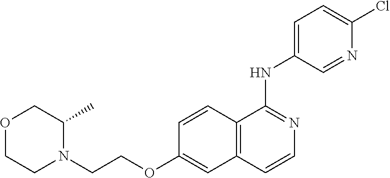 [SMILES: C[C@H]1COCCN1CCOc1ccc2c(Nc3ccc(Cl)nc3)nccc2c1] | (S)-N-(6-chloropyridin-3-yl)-6- (2-(3- methylmorpholino)ethoxy)iso- quinolin-1-amine |
| 322 | 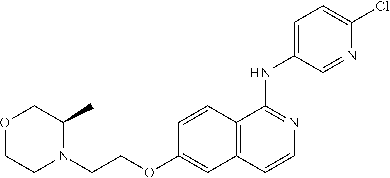 [SMILES: C[C@@H]1COCCN1CCOc1ccc2c(Nc3ccc(Cl)nc3)nccc2c1] | (R)-N-(6-chloropyridin-3-yl)-6- (2-(3- methylmorpholino)ethoxy)iso- quinolin-1-amine |
| 323 | 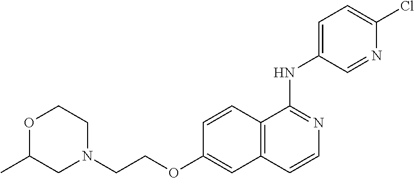 [SMILES: CC1CN(CCOc2ccc3c(Nc4ccc(Cl)nc4)nccc3c2)CCO1] | N-(6-chloropyridin-3-yl)-6-(2- (2- methylmorpholino)ethoxy)iso- quinolin-1-amine |
| 324 | 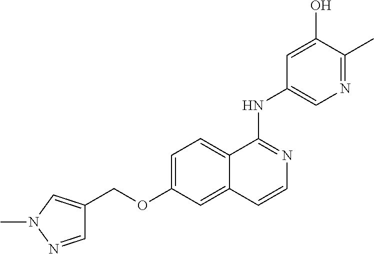 | 2-methyl-5-((6-((1-methyl-1H- pyrazol-4- yl)methoxy)isoquinolin-1- yl)amino)pyridin-3-ol |
| 325 | 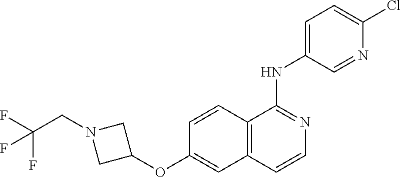 [SMILES: FC(F)(F)CN1CC(Oc2ccc3c(Nc4ccc(Cl)nc4)nccc3c2)C1] | N-(6-chloropyridin-3-yl)-6-((1- (2,2,2-trifluoroethyl)azetidin-3- yl)oxy)isoquinolin-1-amine |
| 326 | 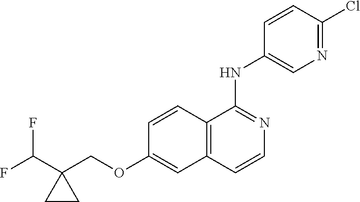 [SMILES: FC(F)C1(COc2ccc3c(Nc4ccc(Cl)nc4)nccc3c2)CC1] | N-(6-chloropyridin-3-yl)-6-((1- (difluoromethyl)cyclopropyl)meth- oxy)isoquinolin-1-amine |
| 327 | 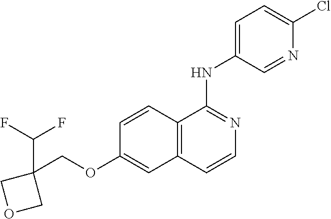 [SMILES: FC(F)C1(COc2ccc3c(Nc4ccc(Cl)nc4)nccc3c2)COC1] | N-(6-chloropyridin-3-yl)-6-((3- (difluoromethyl)oxetan-3- yl)methoxy)isoquinolin-1- amine |
| 328 | 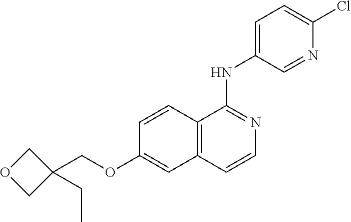 [SMILES: CCC1(COc2ccc3c(Nc4ccc(Cl)nc4)nccc3c2)COC1] | N-(6-chloropyridin-3-yl)-6-((3- ethyloxetan-3- yl)methoxy)isoquinolin-1- amine |
| 329 | 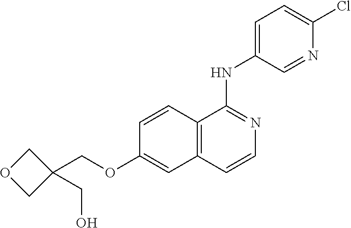 [SMILES: OCC1(COc2ccc3c(Nc4ccc(Cl)nc4)nccc3c2)COC1] | 3-(((1-((6-chloropyridin-3- yl)amino)isoquinolin-6- yl)oxy)methyl)oxetan-3- yl)methanol |
| 330 | 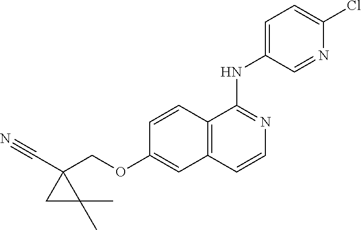 [SMILES: CC1(C)CC1(C#N)COc1ccc2c(Nc3ccc(Cl)nc3)nccc2c1] | 1-(((1-((6-chloropyridin-3- yl)amino)isoquinolin-6- yl)oxy)methyl)-2,2- dimethylcyclopropane-1- carbonitrile 2,2,2- trifluoroacetate |
| 331 | 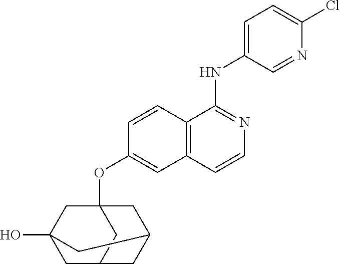 [SMILES: OC12CC3CC(C1)CC(Oc1ccc4c(Nc5ccc(Cl)nc5)nccc4c1)(C3)C2] | 3-((1-((6-chloropyridin-3- yl)amino)isoquinolin-6- yl)oxy)adamantan-1-ol |
| 332 | 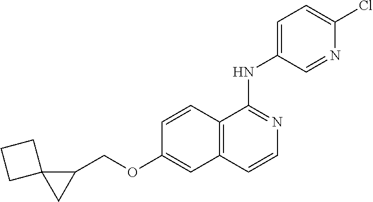 [SMILES: Clc1ccc(Nc2nccc3cc(OCC4CC45CCC5)ccc23)cn1] | N-(6-chloropyridin-3-yl)-6- (spiro[2.3]hexan-1- ylmethoxy)isoquinolin-1- amine |
| 333 | 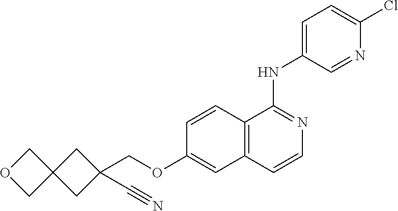 [SMILES: N#CC1(COc2ccc3c(Nc4ccc(Cl)nc4)nccc3c2)CC2(COC2)C1] | 6-(((1-((6-chloropyridin-3- yl)amino)isoquinolin-6- yl)oxy)methyl)-2- oxaspiro[3.3]heptane-6- carbonitrile |
| 334 | 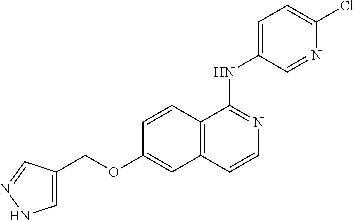 [SMILES: Clc1ccc(Nc2nccc3cc(OCc4cn[nH]c4)ccc23)cn1] | 6-(1-(1H-pyrazol-4-yl)ethoxy)- N-(6-chloropyridin-3- yl)isoquinolin-1-amine |
| 335 | 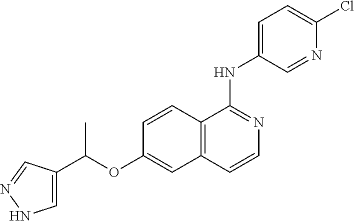 [SMILES: CC(Oc1ccc2c(Nc3ccc(Cl)nc3)nccc2c1)c1cn[nH]c1] | 6-(1-(1H-pyrazol-4-yl)ethoxy)- N-(6-chloropyridin-3- yl)isoquinolin-1-amine |
| 336 | 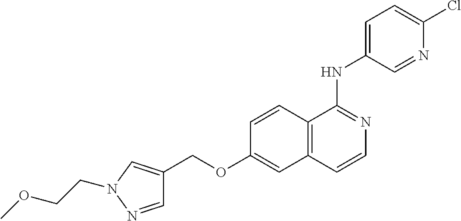 [SMILES: COCCn1cc(COc2ccc3c(Nc4ccc(Cl)nc4)nccc3c2)cn1] | N-(6-chloropyridin-3-yl)-6-((1- (2-methoxyethyl)-1H-pyrazol- 4-yl)methoxy)isoquinolin-1- amine |
| 337 | 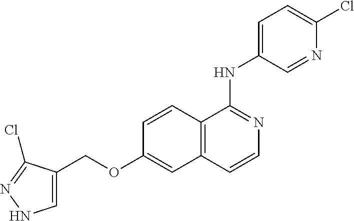 [SMILES: Clc1ccc(Nc2nccc3cc(OCc4c[nH]nc4Cl)ccc23)cn1] | 6-((3-chloro-1H-pyrazol-4- yl)methoxy)-N-(6- chloropyridin-3-yl)isoquinolin- 1-amine |
| 338 | 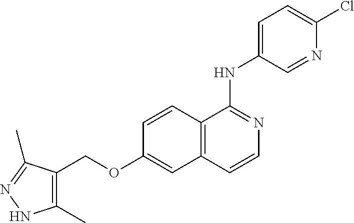 [SMILES: Cc1n[nH]c(C)c1COc1ccc2c(Nc3ccc(Cl)nc3)nccc2c1] | N-(6-chloropyridin-3-yl)-6- ((3,5-dimethyl-1H-pyrazol-4- yl)methoxy)isoquinolin-1- amine |
| 339 | 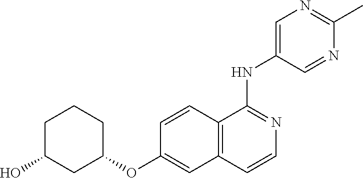 [SMILES: Cc1ncc(Nc2nccc3cc(O[C@H]4CCC[C@@H](O)C4)ccc23)cn1] | (1R,3S)-3-((1-((2- methylpyrimidin-5- yl)amino)isoquinolin-6- yl)oxy)cyclohexan-1-ol |
| 340 | 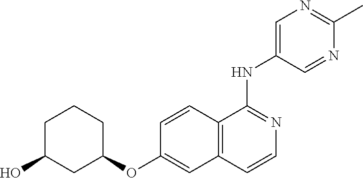 [SMILES: Cc1ncc(Nc2nccc3cc(O[C@@H]4CCC[C@H](O)C4)ccc23)cn1] | (1S,3R)-3-((1-((2- methylpyrimidin-5- yl)amino)isoquinolin-6- yl)oxy)cyclohexan-1-ol |
| 341 | 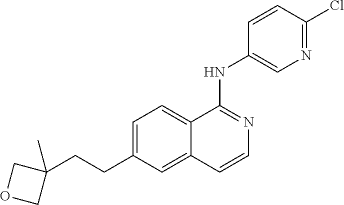 [SMILES: CC1(CCc2ccc3c(Nc4ccc(Cl)nc4)nccc3c2)COC1] | N-(6-chloropyridin-3-yl)-6-(2- (3-methyloxetan-3- yl)ethyl)isoquinolin-1-amine |
| 342 | 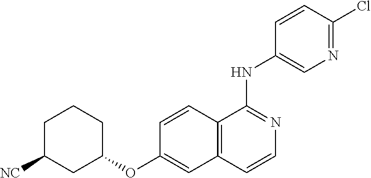 [SMILES: N#C[C@H]1CCC[C@H](Oc2ccc3c(Nc4ccc(Cl)nc4)nccc3c2)C1] | (1S,3S)-3-((1-((6- chloropyridin-3- yl)amino)isoquinolin-6- yl)oxy)cyclohexane-1- carbonitrile |
| 343 | 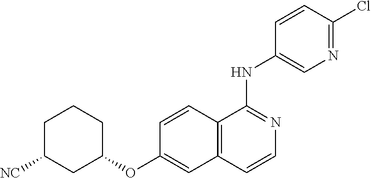 [SMILES: N#C[C@@H]1CCC[C@H](Oc2ccc3c(Nc4ccc(Cl)nc4)nccc3c2)C1] | (1R,3S)-3-((1-((6- chloropyridin-3- yl)amino)isoquinolin-6- yl)oxy)cyclohexane-1- carbonitrile |
| 344 | 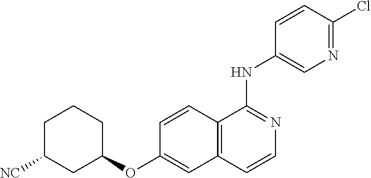 [SMILES: N#C[C@@H]1CCC[C@@H](Oc2ccc3c(Nc4ccc(Cl)nc4)nccc3c2)C1] | (1R,3R)-3-((1-((6- chloropyridin-3- yl)amino)isoquinolin-6- yl)oxy)cyclohexane-1- carbonitrile |
| 345 | 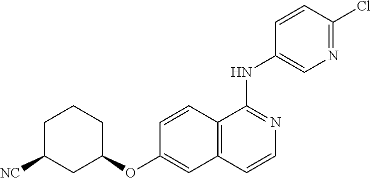 [SMILES: N#C[C@H]1CCC[C@@H](Oc2ccc3c(Nc4ccc(Cl)nc4)nccc3c2)C1] | (1S,3R)-3-((1-((6- chloropyridin-3- yl)amino)isoquinolin-6- yl)oxy)cyclohexane-1- carbonitrile |
| 346 | 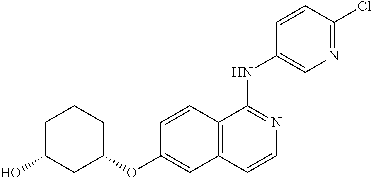 [SMILES: O[C@@H]1CCC[C@H](Oc2ccc3c(Nc4ccc(Cl)nc4)nccc3c2)C1] | (1R,3S)-3-((1-((6- chloropyridin-3- yl)amino)isoquinolin-6- yl)oxy)cyclohexan-1-ol |
| 347 | 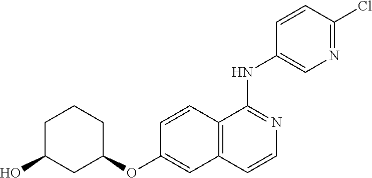 [SMILES: O[C@H]1CCC[C@@H](Oc2ccc3c(Nc4ccc(Cl)nc4)nccc3c2)C1] | (1S,3R)-3-((1-((6- chloropyridin-3- yl)amino)isoquinolin-6- yl)oxy)cyclohexan-1-ol |
| 348 | 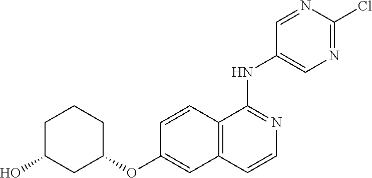 [SMILES: O[C@@H]1CCC[C@H](Oc2ccc3c(Nc4cnc(Cl)nc4)nccc3c2)C1] | cis-3-((1-((2-chloropyrimidin- 5-yl)amino)isoquinolin-6- yl)oxy)cyclohexan-1-ol |
| 349 | 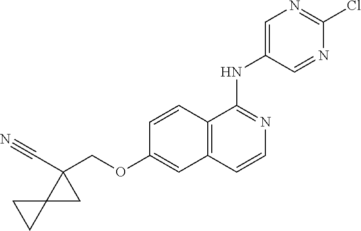 [SMILES: N#CC1(COc2ccc3c(Nc4cnc(Cl)nc4)nccc3c2)CC12CC2] | 1-(((1-((2-chloropyrimidin-5- yl)amino)isoquinolin-6- yl)oxy)methyl)spiro[2.2]pentane- 1-carbonitrile |
| 350 | 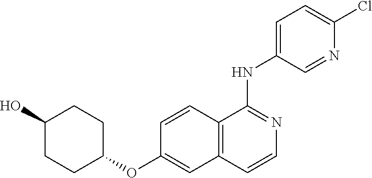 [SMILES: O[C@H]1CC[C@H](Oc2ccc3c(Nc4ccc(Cl)nc4)nccc3c2)CC1] | (1R,4R)-4-((1-((6- chloropyridin-3- yl)amino)isoquinolin-6- yl)oxy)cyclohexan-1-ol |
| 351 | 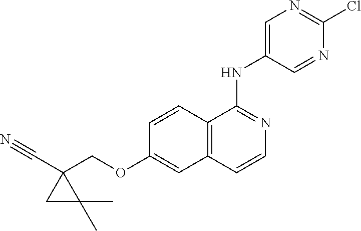 [SMILES: CC1(C)CC1(C#N)COc1ccc2c(Nc3cnc(Cl)nc3)nccc2c1] | 1-(((1-((2-chloropyrimidin-5- yl)amino)isoquinolin-6- yl)oxy)methyl)-2,2- dimethylcyclopropane-1- carbonitrile |
| 352 | 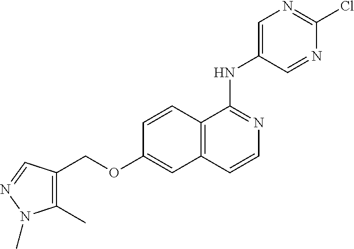 [SMILES: Cc1c(COc2ccc3c(Nc4cnc(Cl)nc4)nccc3c2)cnn1C] | N-(2-chloropyrimidin-5-yl)-6- ((1,5-dimethyl-1H-pyrazol-4- yl)methoxy)isoquinolin-1- amine |
| 353 | 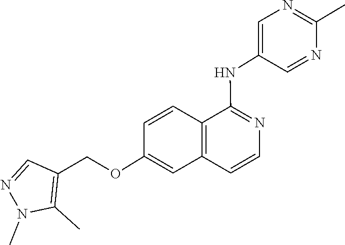 [SMILES: Cc1ncc(Nc2nccc3cc(OCc4cnn(C)c4C)ccc23)cn1] | 6-((1,5-dimethyl-1H-pyrazol- 4-yl)methoxy)-N-(2- methylpyrimidin-5- yl)isoquinolin-1-amine |
| 354 | 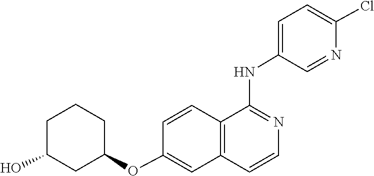 [SMILES: O[C@@H]1CCC[C@@H](Oc2ccc3c(Nc4ccc(Cl)nc4)nccc3c2)C1] | trans-3-((1-((6-chloropyridin- 3-yl)amino)isoquinolin-6- yl)oxy)cyclohexan-1-ol |
| 355 | 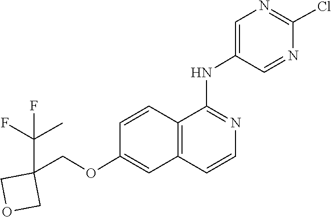 [SMILES: CC(F)(F)C1(COc2ccc3c(Nc4cnc(Cl)nc4)nccc3c2)COC1] | N-(2-chloropyrimidin-5-yl)-6- ((3-(1,1-difluoroethyl)oxetan- 3-yl)methoxy)isoquinolin-1- amine |
| 356 | 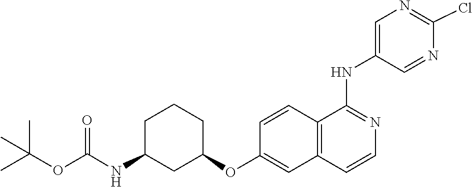 [SMILES: CC(C)(C)OC(=O)N[C@H]1CCC[C@@H](Oc2ccc3c(Nc4cnc(Cl)nc4)nccc3c2)C1] | tert-butyl ((1S,3R)-3-((1-((2- chloropyrimidin-5- yl)amino)isoquinolin-6- yl)oxy)cyclohexyl)carbamate |
| 357 | 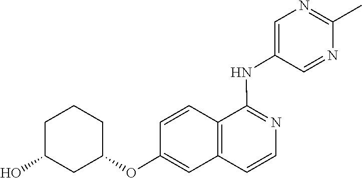 [SMILES: Cc1ncc(Nc2nccc3cc(O[C@H]4CCC[C@@H](O)C4)ccc23)cn1] | cis-3-((1-((2-methylpyrimidin- 5-yl)amino)isoquinolin-6- yl)oxy)cyclohexan-1-ol |
| 358 | 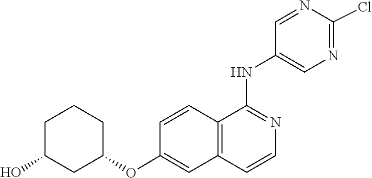 [SMILES: O[C@@H]1CCC[C@H](Oc2ccc3c(Nc4cnc(Cl)nc4)nccc3c2)C1] | (1R,3S)-3-((1-((2- chloropyrimidin-5- yl)amino)isoquinolin-6- yl)oxy)cyclohexan-1-ol |
| 359 | 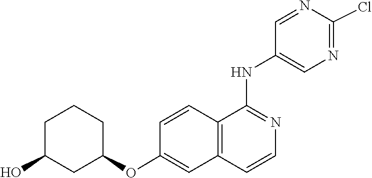 [SMILES: O[C@H]1CCC[C@@H](Oc2ccc3c(Nc4cnc(Cl)nc4)nccc3c2)C1] | (1S,3R)-3-((1-((2- chloropyrimidin-5- yl)amino)isoquinolin-6- yl)oxy)cyclohexan-1-ol |
| 360 | 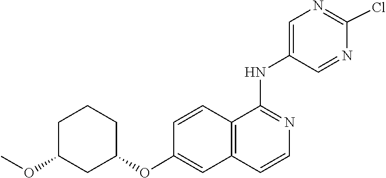 [SMILES: CO[C@@H]1CCC[C@H](Oc2ccc3c(Nc4cnc(Cl)nc4)nccc3c2)C1] | cis-N-(2-chloropyrimidin-5-yl)- 6-(((1S,3R)-3- methoxycyclohexyl)oxy)iso- quinolin-1-amine |
| 361 | 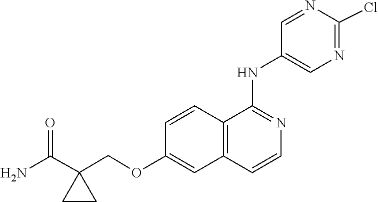 [SMILES: NC(=O)C1(COc2ccc3c(Nc4cnc(Cl)nc4)nccc3c2)CC1] | 1-(((1-((2-chloropyrimidin-5- yl)amino)isoquinolin-6- yl)oxy)methyl)cyclopropane- 1-carboxamide |
| 362 | 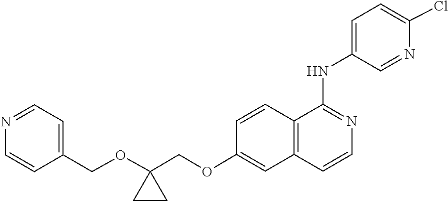 [SMILES: Clc1ccc(Nc2nccc3cc(OCC4(OCc5ccncc5)CC4)ccc23)cn1] | N-(6-chloropyridin-3-yl)-6-((1- (pyridin-4- ylmethoxy)cyclopropyl)meth- oxy)isoquinolin-1-amine |
| 363 | 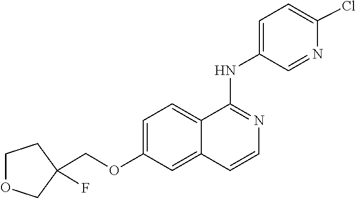 [SMILES: FC1(COc2ccc3c(Nc4ccc(Cl)nc4)nccc3c2)CCOC1] | N-(6-chloropyridin-3-yl)-6-((3- fluorotetrahydrofuran-3- yl)methoxy)isoquinolin-1- amine |
| 364 | 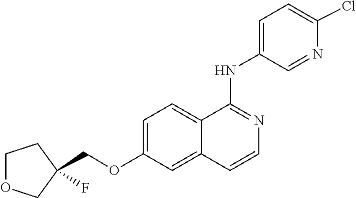 [SMILES: F[C@@]1(COc2ccc3c(Nc4ccc(Cl)nc4)nccc3c2)CCOC1] | (R)-N-(6-chloropyridin-3-yl)-6- ((3-fluorotetrahydrofuran-3- yl)methoxy)isoquinolin-1- amine |
| 365 | 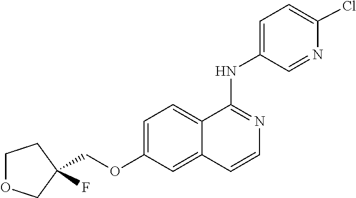 [SMILES: F[C@]1(COc2ccc3c(Nc4ccc(Cl)nc4)nccc3c2)CCOC1] | (S)-N-(6-chloropyridin-3-yl)-6- ((3-fluorotetrahydrofuran-3- yl)methoxy)isoquinolin-1- amine |
| 366 | 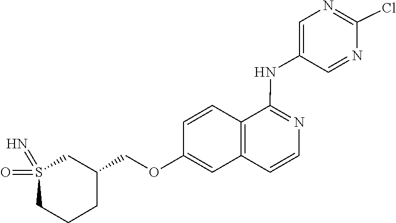 [SMILES: N=[S@]1(=O)CCC[C@@H](COc2ccc3c(Nc4cnc(Cl)nc4)nccc3c2)C1] | cis-3-(((1-((2-chloropyrimidin- 5-yl)amino)isoquinolin-6- yl)oxy)methyl)-1- iminohexahydro-1λ⁶-thiopyran 1-oxide |
| 367 | 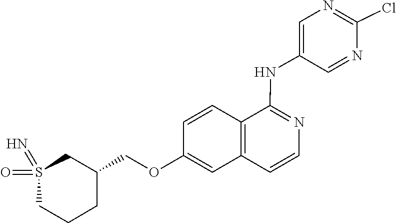 [SMILES: N=[S@@]1(=O)CCC[C@@H](COc2ccc3c(Nc4cnc(Cl)nc4)nccc3c2)C1] | trans-3-(((1-((2- chloropyrimidin-5- yl)amino)isoquinolin-6- yl)oxy)methyl)-1- iminohexahydro-1λ⁶-thiopyran 1-oxide |
| 368 | 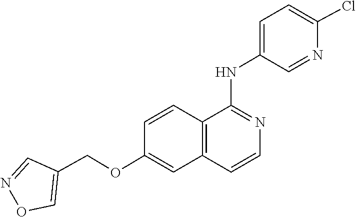 [SMILES: Clc1ccc(Nc2nccc3cc(OCc4cnoc4)ccc23)cn1] | N-(6-chloropyridin-3-yl)-6- (isoxazol-4- ylmethoxy)isoquinolin-1- amine |
| 369 | 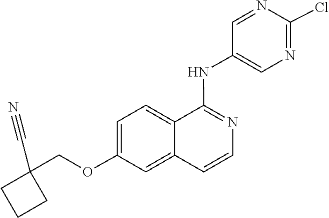 [SMILES: N#CC1(COc2ccc3c(Nc4cnc(Cl)nc4)nccc3c2)CCC1] | 1-(((1-((2-chloropyrimidin-5- yl)amino)isoquinolin-6- yl)oxy)methyl)cyclobutane-1- carbonitrile |
| 370 | 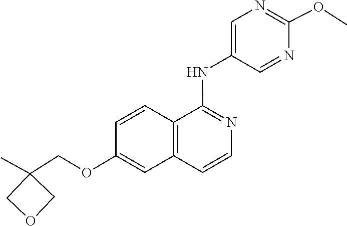 | N-(2-methoxypyrimidin-5-yl)- 6-((3-methyloxetan-3- yl)methoxy)isoquinolin-1- amine |
| 371 | 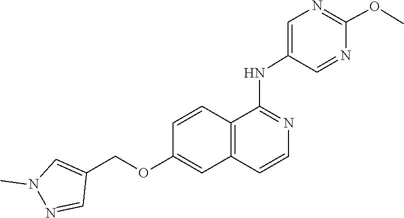 | N-(2-methoxypyrimidin-5-yl)- 6-((1-methyl-1H-pyrazol-4- yl)methoxy)isoquinolin-1- amine |
| 372 | 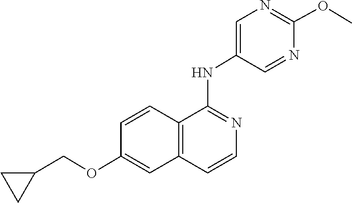 [SMILES: COc1ncc(Nc2nccc3cc(OCC4CC4)ccc23)cn1] | 6-(cyclopropylmethoxy)-N-(2- methoxypyrimidin-5- yl)isoquinolin-1-amine |
| 373 | 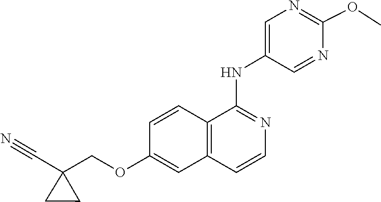 [SMILES: COc1ncc(Nc2nccc3cc(OCC4(C#N)CC4)ccc23)cn1] | 1-(((1-((2-methoxypyrimidin-5- yl)amino)isoquinolin-6- yl)oxy)methyl)cyclopropane- 1-carbonitrile |
| 374 | 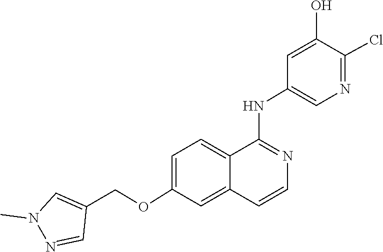 [SMILES: Cn1cc(COc2ccc3c(Nc4cnc(Cl)c(O)c4)nccc3c2)cn1] | 2-chloro-5-((6-((1-methyl-1H- pyrazol-4- yl)methoxy)isoquinolin-1- yl)amino)pyridin-3-ol |
| 375 | 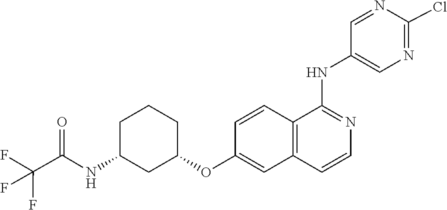 [SMILES: O=C(N[C@@H]1CCC[C@H](Oc2ccc3c(Nc4cnc(Cl)nc4)nccc3c2)C1)C(F)(F)F] | N-((1R,3S)-3-((1-((2- chloropyrimidin-5- yl)amino)isoquinolin-6- yl)oxy)cyclohexyl)-2,2,2- trifluoroacetamide |
| 376 | 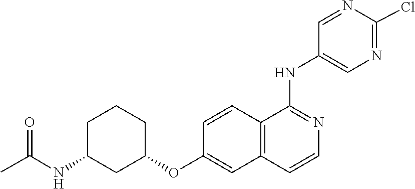 [SMILES: CC(=O)N[C@@H]1CCC[C@H](Oc2ccc3c(Nc4cnc(Cl)nc4)nccc3c2)C1] | N-((1R,3S)-3-((1-((2- chloropyrimidin-5- yl)amino)isoquinolin-6- yl)oxy)cyclohexyl)acetamide |
| 377 | 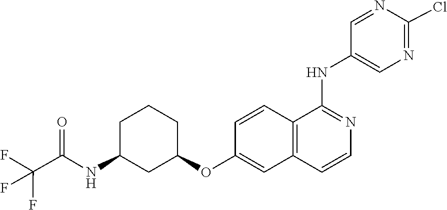 [SMILES: O=C(N[C@H]1CCC[C@@H](Oc2ccc3c(Nc4cnc(Cl)nc4)nccc3c2)C1)C(F)(F)F] | N-((1S,3R)-3-((1-((2- chloropyrimidin-5- yl)amino)isoquinolin-6- yl)oxy)cyclohexyl)-2,2,2- trifluoroacetamide |
| 378 | 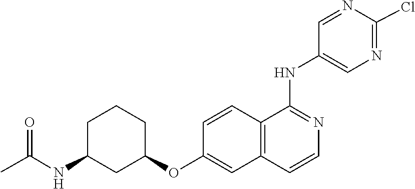 [SMILES: CC(=O)N[C@H]1CCC[C@@H](Oc2ccc3c(Nc4cnc(Cl)nc4)nccc3c2)C1] | N-((1S,3R)-3-((1-((2- chloropyrimidin-5- yl)amino)isoquinolin-6- yl)oxy)cyclohexyl)acetamide |
| 379 | 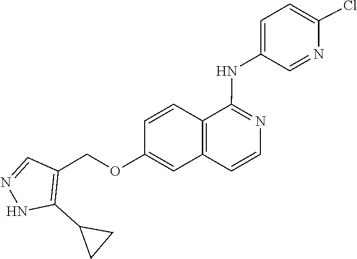 [SMILES: Clc1ccc(Nc2nccc3cc(OCc4cn[nH]c4C4CC4)ccc23)cn1] | N-(6-chloropyridin-3-yl)-6-((5- cyclopropyl-1H-pyrazol-4- yl)methoxy)isoquinolin-1- amine |
| 380 | 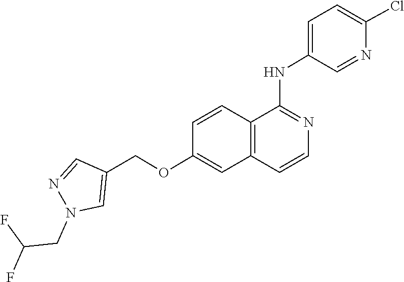 [SMILES: FC(F)Cn1cc(COc2ccc3c(Nc4ccc(Cl)nc4)nccc3c2)cn1] | N-(6-chloropyridin-3-yl)-6-((1- (2,2-difluoroethyl)-1H-pyrazol- 4-yl)methoxy)isoquinolin-1- amine |
| 381 | 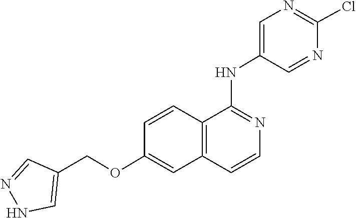 [SMILES: Clc1ncc(Nc2nccc3cc(OCc4cn[nH]c4)ccc23)cn1] | 6-((1H-pyrazol-4-yl)methoxy)- N-(2-chloropyimidin-5- yl)isoquinolin-1-amine |
| 382 | 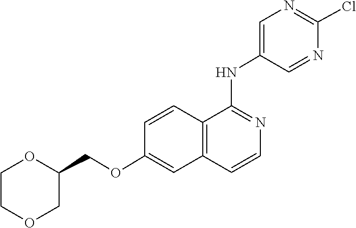 [SMILES: Clc1ncc(Nc2nccc3cc(OC[C@H]4COCCO4)ccc23)cn1] | (R)-6-((1,4-dioxan-2- yl)methoxy)-N-(2- chloropyrimidin-5- yl)isoquinolin-1-amine |
| 383 | 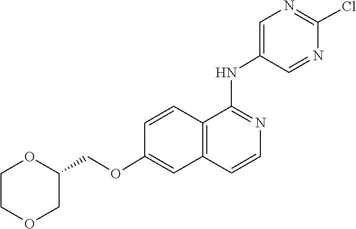 [SMILES: Clc1ncc(Nc2nccc3cc(OC[C@@H]4COCCO4)ccc23)cn1] | (S)-6-((1,4-dioxan-2- yl)methoxy)-N-(2- chloropyrimidin-5- yl)isoquinolin-1-amine |
| 384 | 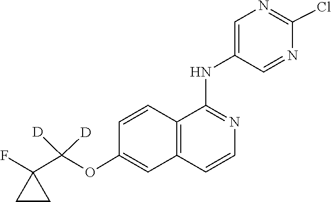 [SMILES: [2H]C([2H])(Oc1ccc2c(Nc3cnc(Cl)nc3)nccc2c1)C1(F)CC1] | N-(2-chloropyrimidin-5-yl)-6- ((1- fluorocyclopropyl)methoxy- d₂)isoquinolin-1-amine |
| 385 | 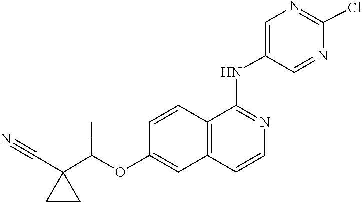 [SMILES: CC(Oc1ccc2c(Nc3cnc(Cl)nc3)nccc2c1)C1(C#N)CC1] | 1-(1-((1-((2-chloropyrimidin-5- yl)amino)isoquinolin-6- yl)oxy)ethyl)cyclopropane-1- carbonitrile |
| 386 | 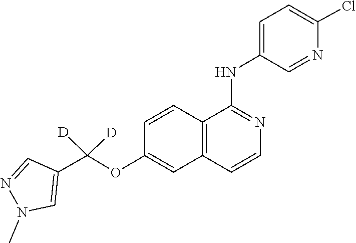 [SMILES: [2H]C([2H])(Oc1ccc2c(Nc3ccc(Cl)nc3)nccc2c1)c1cnn(C)c1] | N-(6-chloropyridin-3-yl)-6-((1- methyl-1H-pyrazol-4- yl)methoxy-d₂)isoquinolin-1- amine |
| 387 | 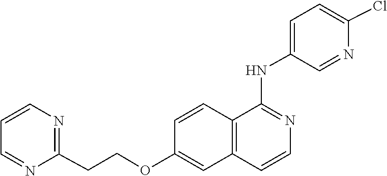 [SMILES: Clc1ccc(Nc2nccc3cc(OCCc4ncccn4)ccc23)cn1] | N-(6-chloropyridin-3-yl)-6-(2- (pyrimidin-2- yl)ethoxy)isoquinolin-1-amine |
| 388 | 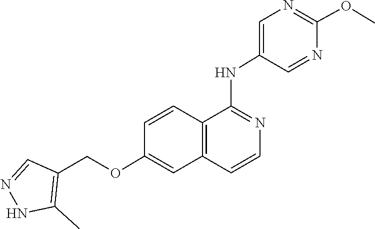 [SMILES: COc1ncc(Nc2nccc3cc(OCc4cn[nH]c4C)ccc23)cn1] | N-(2-methoxypyrimidin-5-yl)- 6-((5-methyl-1H-pyrazol-4- yl)methoxy)isoquinolin-1- amine |
| 389 | 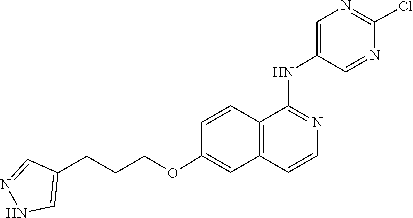 [SMILES: Clc1ncc(Nc2nccc3cc(OCCCc4cn[nH]c4)ccc23)cn1] | 6-(3-(1H-pyrazol-4- yl)propoxy)-N-(2- chloropyrimidin-5- yl)isoquinolin-1-amine |
| 390 | 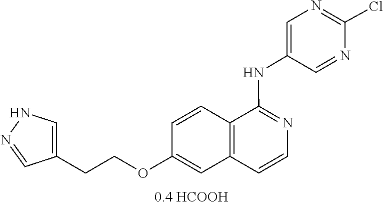 [SMILES: Clc1ncc(Nc2nccc3cc(OCCc4cn[nH]c4)ccc23)cn1] | 6-(2-(1H-pyrazol-4-yl)ethoxy)- N-(2-chloropyrimidin-5- yl)isoquinolin-1-amine |
| 391 | 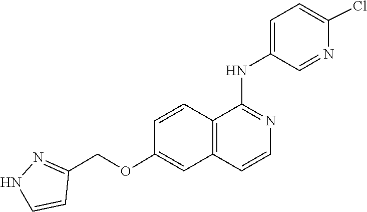 [SMILES: Clc1ccc(Nc2nccc3cc(OCc4cc[nH]n4)ccc23)cn1] | 6-((1H-pyrazol-3-yl)methoxy)- N-(6-chloropyridin-3- yl)isoquinolin-1-amine |
| 392 | 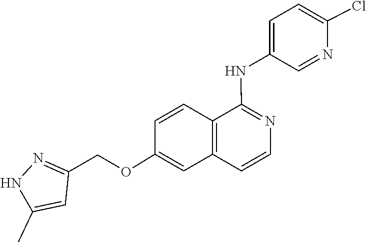 [SMILES: Cc1cc(COc2ccc3c(Nc4ccc(Cl)nc4)nccc3c2)n[nH]1] | N-(6-chloropyridin-3-yl)-6-((5- methyl-1H-pyrazol-3- yl)methoxy)isoquinolin-1- amine |
| 393 | 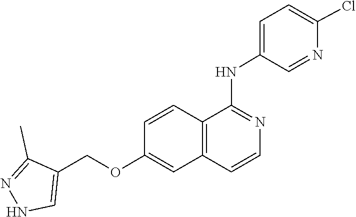 [SMILES: Cc1n[nH]cc1COc1ccc2c(Nc3ccc(Cl)nc3)nccc2c1] | N-(6-chloropyridin-3-yl)-6-((3- methyl-1H-pyrazol-4- yl)methoxy)isoquinolin-1- amine |
| 394 | 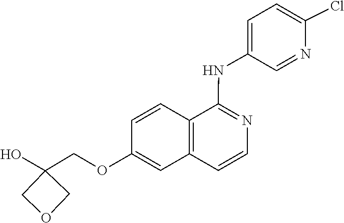 [SMILES: OC1(COc2ccc3c(Nc4ccc(Cl)nc4)nccc3c2)COC1] | 3-(((1-((6-chloropyridin-3- yl)amino)isoquinolin-6- yl)oxy)methyl)oxetan-3-ol |
| 395 | 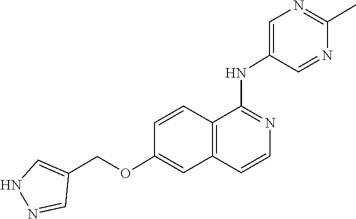 [SMILES: Cc1ncc(Nc2nccc3cc(OCc4cn[nH]c4)ccc23)cn1] | 6-((1H-pyrazol-4-yl)methoxy)- N-(2-methylpyrimidin-5- yl)isoquinolin-1-amine |
| 396 | 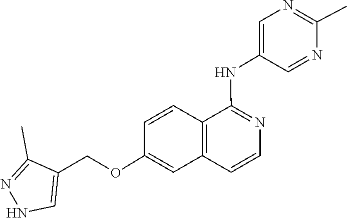 [SMILES: Cc1ncc(Nc2nccc3cc(OCc4c[nH]nc4C)ccc23)cn1] | 6-((3-methyl-1H-pyrazol-4- yl)methoxy)-N-(2- methylpyrimidin-5- yl)isoquinolin-1-amine |
| 397 | 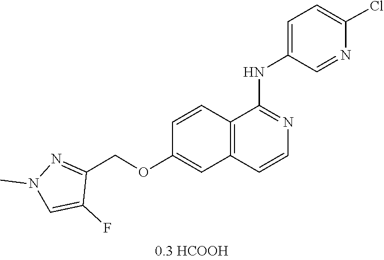 [SMILES: Cn1cc(F)c(COc2ccc3c(Nc4ccc(Cl)nc4)nccc3c2)n1] | N-(6-chloropyridin-3-yl)-6-((4- fluoro-1-methyl-1H-pyrazol-3- yl)methoxy)isoquinolin-1- amine |
| 398 | 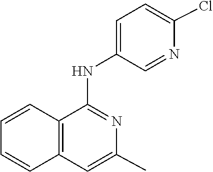 [SMILES: Cc1cc2ccccc2c(Nc2ccc(Cl)nc2)n1] | N-(6-chloropyridin-3-yl)-3- methylisoquinolin-1-amine |
| 399 | 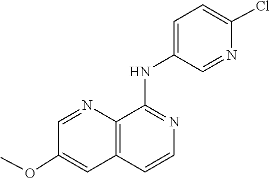 [SMILES: COc1cnc2c(Nc3ccc(Cl)nc3)nccc2c1] | N-(6-chloropyridin-3-yl)-3- methoxy-1,7-naphthyridin-8- amine |
| 400 | 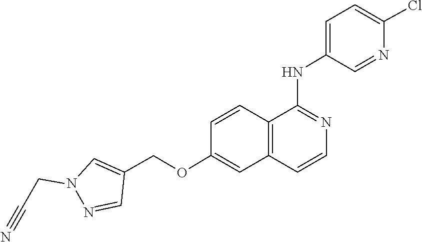 [SMILES: N#CCn1cc(COc2ccc3c(Nc4ccc(Cl)nc4)nccc3c2)cn1] | 2-(4-(((1-((6-chloropyridin-3- yl)amino)isoquinolin-6- yl)oxy)methyl)-1H-pyrazol-1- yl)acetonitrile |
| 401 | 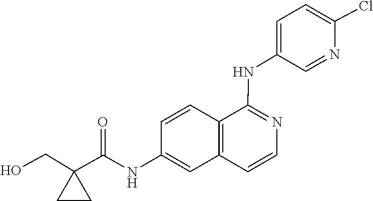 [SMILES: O=C(Nc1ccc2c(Nc3ccc(Cl)nc3)nccc2c1)C1(CO)CC1] | N-(1-((6-chloropyridin-3- yl)amino)isoquinolin-6-yl)-1- (hydroxymethyl)cyclopropane- 1-carboxamide |
| 402 | 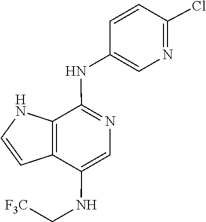 [SMILES: FC(F)(F)CNc1cnc(Nc2ccc(Cl)nc2)c2[nH]ccc12] | N⁷-(6-chloropyridin-3-yl)-N⁴- (2,2,2-trifluoroethyl)-1H- pyrrolo[2,3-c]pyridine-4,7- diamine |
| 403 | 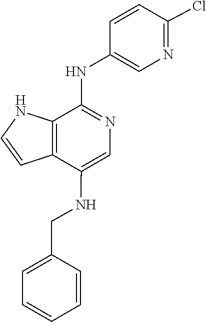 [SMILES: Clc1ccc(Nc2ncc(NCc3ccccc3)c3cc[nH]c23)cn1] | N⁴-benzyl-N⁷-(6-chloropyridin- 3-yl)-1H-pyrrolo[2,3- c]pyridine-4,7-diamine |
| 404 | 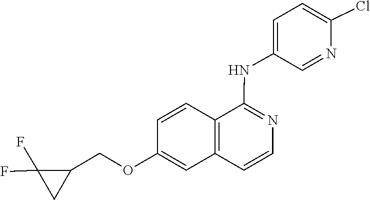 [SMILES: FC1(F)CC1COc1ccc2c(Nc3ccc(Cl)nc3)nccc2c1] | N-(6-chloropyridin-3-yl)-6- ((2,2- difluorocyclopropyl)methoxy)iso- quinolin-1-amine |
| 405 | 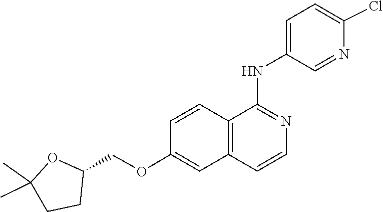 [SMILES: CC1(C)CC[C@@H](COc2ccc3c(Nc4ccc(Cl)nc4)nccc3c2)O1] | (S)-N-(6-chloropyridin-3-yl)-6- ((5,5-dimethyltetrahydrofuran- 2-yl)methoxy)isoquinolin-1- amine |
| 406 | 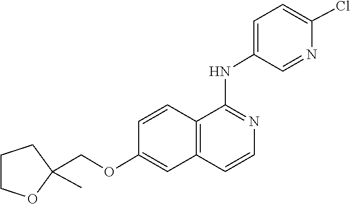 [SMILES: CC1(COc2ccc3c(Nc4ccc(Cl)nc4)nccc3c2)CCCO1] | N-(6-chloropyridin-3-yl)-6-((2- methyltetrahydrofuran-2- yl)methoxy)isoquinolin-1- amine |
| 407 | 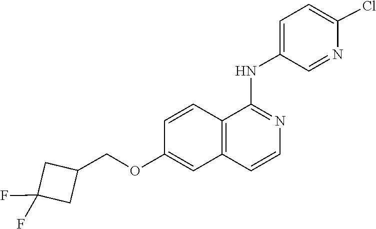 [SMILES: FC1(F)CC(COc2ccc3c(Nc4ccc(Cl)nc4)nccc3c2)C1] | N-(6-chloropyridin-3-yl)-6- ((3,3- difluorocyclobutyl)methoxy)iso- quinolin-1-amine |
| 408 | 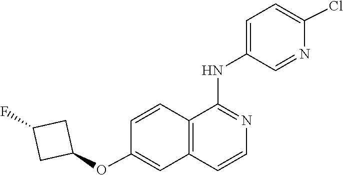 [SMILES: F[C@H]1C[C@H](Oc2ccc3c(Nc4ccc(Cl)nc4)nccc3c2)C1] | N-(6-chloropyridin-3-yl)-6- ((1R,3R)-3- fluorocyclobutoxy)isoquinolin- 1-amine |
| 409 | 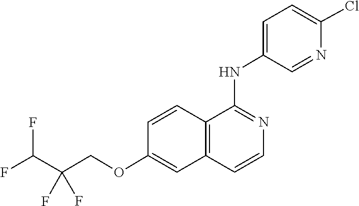 [SMILES: FC(F)C(F)(F)COc1ccc2c(Nc3ccc(Cl)nc3)nccc2c1] | N-(6-chloropyridin-3-yl)-6- (2,2,3,3- tetrafluoropropoxy)isoquinolin- 1-amine |
| 410 | 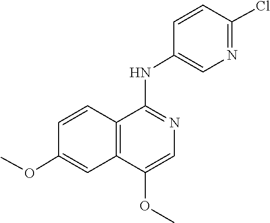 [SMILES: COc1ccc2c(Nc3ccc(Cl)nc3)ncc(OC)c2c1] | N-(6-chloropyridin-3-yl)-4,6- dimethoxyisoquinolin-1-amine |
| 411 | 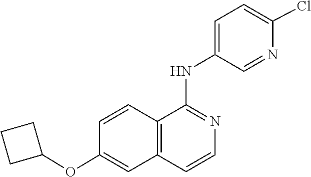 [SMILES: Clc1ccc(Nc2nccc3cc(OC4CCC4)ccc23)cn1] | N-(6-chloropyridin-3-yl)-6- cyclobutoxyisoquinolin-1- amine |
| 412 | 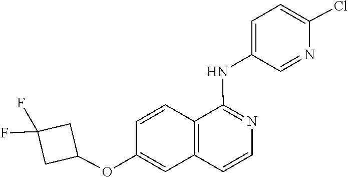 [SMILES: FC1(F)CC(Oc2ccc3c(Nc4ccc(Cl)nc4)nccc3c2)C1] | N-(6-chloropyridin-3-yl)-6- (3,3- difluorocyclobutoxy)isoquinolin- 1-amine |
| 413 | 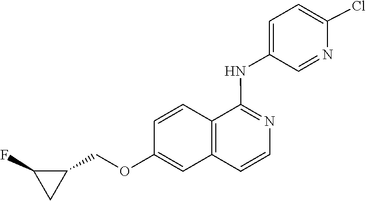 [SMILES: F[C@@H]1C[C@H]1COc1ccc2c(Nc3ccc(Cl)nc3)nccc2c1] | N-(6-chloropyridin-3-yl)-6- (((1S,2R)-2- fluorocyclopropyl)methoxy)iso- quinolin-1-amine |
| 414 | 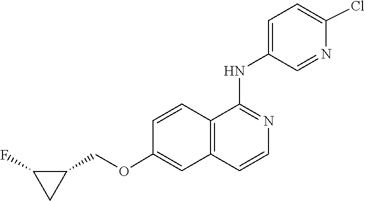 [SMILES: F[C@H]1C[C@H]1COc1ccc2c(Nc3ccc(Cl)nc3)nccc2c1] | N-(6-chloropyridin-3-yl)-6- (((1S,2S)-2- fluorocyclopropyl)methoxy)iso- quinolin-1-amine |
| 415 | 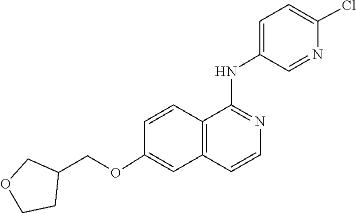 [SMILES: Clc1ccc(Nc2nccc3cc(OCC4CCOC4)ccc23)cn1] | N-(6-chloropyridin-3-yl)-6- ((tetrahydrofuran-3- yl)methoxy)isoquinolin-1- amine |
| 416 | 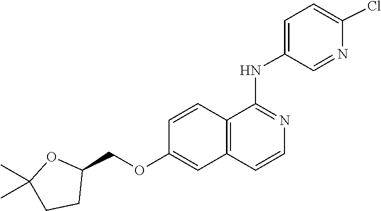 [SMILES: CC1(C)CC[C@H](COc2ccc3c(Nc4ccc(Cl)nc4)nccc3c2)O1] | (R)-N-(6-chloropyridin-3-yl)-6- ((5,5-dimethyltetrahydrofuran- 2-yl)methoxy)isoquinolin-1- amine |
| 417 | 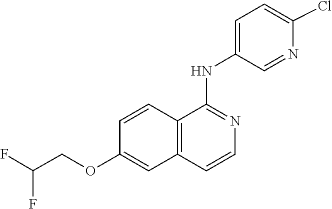 [SMILES: FC(F)COc1ccc2c(Nc3ccc(Cl)nc3)nccc2c1] | N-(6-chloropyridin-3-yl)-6- (2,2- difluoroethoxy)isoquinolin-1- amine |
| 418 | 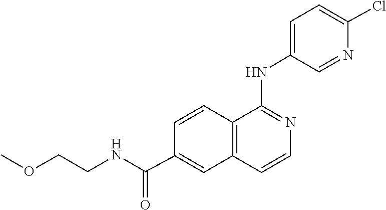 [SMILES: COCCNC(=O)c1ccc2c(Nc3ccc(Cl)nc3)nccc2c1] | 1-((6-chloropyridin-3- yl)amino)-N-(2- methoxyethyl)isoquinoline-6- carboxamide |
| 419 | 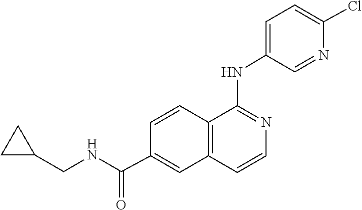 [SMILES: O=C(NCC1CC1)c1ccc2c(Nc3ccc(Cl)nc3)nccc2c1] | 1-((6-chloropyridin-3- yl)amino)-N- (cyclopropylmethyl)isoquinoline- 6-carboxamide |
| 420 | 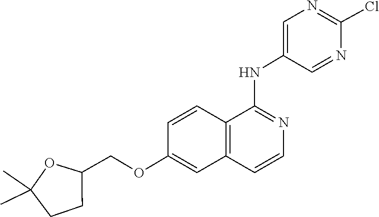 [SMILES: CC1(C)CCC(COc2ccc3c(Nc4cnc(Cl)nc4)nccc3c2)O1] | N-(2-chloropyrimidin-5-yl)-6- ((5,5-dimethyltetrahydrofuran- 2-yl)methoxy)isoquinolin-1- amine |
| 421 | 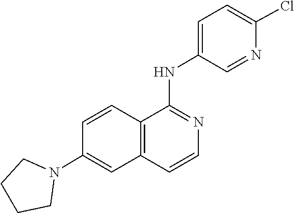 [SMILES: Clc1ccc(Nc2nccc3cc(N4CCCC4)ccc23)cn1] | N-(6-chloropyridin-3-yl)-6- (pyrrolidin-1-yl)isoquinolin-1- amine |
| 422 | 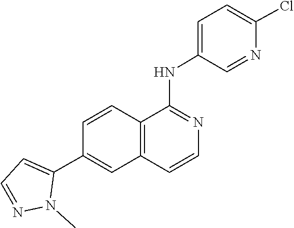 [SMILES: Cn1nccc1-c1ccc2c(Nc3ccc(Cl)nc3)nccc2c1] | N-(6-chloropyridin-3-yl)-6-(1- methyl-1H-pyrazol-5- yl)isoquinolin-1-amine |
| 423 | 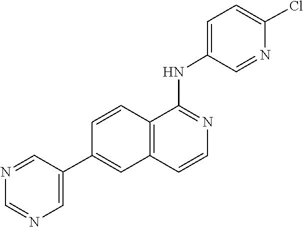 [SMILES: Clc1ccc(Nc2nccc3cc(-c4cncnc4)ccc23)cn1] | N-(6-chloropyridin-3-yl)-6- (pyrimidin-5-yl)isoquinolin-1- amine |
| 424 | 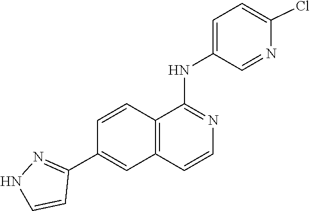 [SMILES: Clc1ccc(Nc2nccc3cc(-c4cc[nH]n4)ccc23)cn1] | N-(6-chloropyridin-3-yl)-6- (1H-pyrazol-3-yl)isoquinolin- 1-amine |
| 425 | 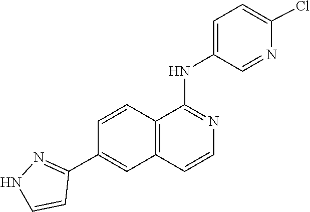 [SMILES: Clc1ccc(Nc2nccc3cc(-c4cc[nH]n4)ccc23)cn1] | 6-((2H-1,2,3-triazol-2- yl)methyl)-N-(6-chloropyridin- 3-yl)isoquinolin-1-amine |
| 426 | 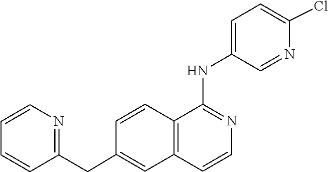 [SMILES: Clc1ccc(Nc2nccc3cc(Cc4ccccn4)ccc23)cn1] | N-(6-chloropyridin-3-yl)-6- (pyridin-2- ylmethyl)isoquinolin-1-amine |
| 427 | 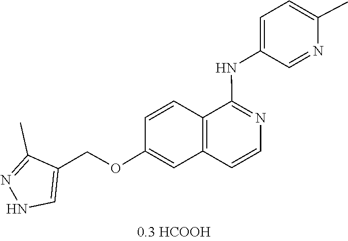 [SMILES: Cc1ccc(Nc2nccc3cc(OCc4c[nH]nc4C)ccc23)cn1] | 6-((3-methyl-1H-pyrazol-4- yl)methoxy)-N-(6- methylpyridin-3-yl)isoquinolin- 1-amine |

Another embodiment of the disclosure is a pharmaceutical composition comprising a pharmaceutically acceptable excipient and a therapeutically effective amount of a compound of Formula (I), as described above in the Brief Summary, as a stereoisomer, enantiomer or tautomer thereof or mixtures thereof; or a pharmaceutically acceptable salt, solvate or prodrug thereof.

Another embodiment of the disclosure is a method of treating a disease or condition in a mammal modulated by a voltage-gated potassium channel, wherein the method comprises administering to a mammal in need thereof a therapeutically effective amount of a compound of Formula (I), as described above in the Brief Summary, as a stereoisomer, enantiomer, or tautomer thereof or mixtures thereof; or a pharmaceutically acceptable salt, solvate or prodrug thereof.

Another embodiment of the disclosure is a method of using the compounds of Formula (I) as standards or controls in in vitro or in vivo assays in determining the efficacy of test compounds in modulating voltage-dependent potassium channels.

Specific embodiments of the compounds of the disclosure are described in more detail below in the Preparation of the Compounds.

### Utility and Testing of Compounds

In an embodiment, the present disclosure is directed to compounds of Formula (I), as individual stereoisomers, enantiomers, or tautomers thereof or mixtures thereof; or pharmaceutically acceptable salts, solvates or prodrugs thereof, which are useful in treating seizure disorders, for example, epilepsy and/or epileptic seizure disorders, in a mammal, preferably a human.

In another embodiment, compounds of Formula (I), as individual stereoisomers, enantiomers or tautomers thereof or mixtures thereof; or pharmaceutically acceptable salts, solvates or prodrugs thereof, disclosed herein are useful in treating epilepsy, partial seizures (such as simple, complex, secondary generalized, and focal onset), generalized seizures (such as absence, myoclonic, atonic, tonic and tonic clonic), and disorders including photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures plus (GEFS+), Rett syndrome, multiple sclerosis, Alzheimer's disease, autism, ataxia, hypotonia and paroxysmal dyskinesia.

The present disclosure readily affords many different means for identification of potassium channel modulating agents that are useful as therapeutic agents. Identification of modulators of potassium channels can be assessed using a variety of in vitro and in vivo assays, e.g., measuring current, measuring membrane potential, measuring ion flux, (e.g., potassium), measuring potassium concentration, measuring second messengers and transcription levels, and using voltage-sensitive dyes, radioactive tracers, and patch-clamp electrophysiology.

One such protocol involves the screening of chemical agents for ability to modulate the activity of a potassium channel thereby identifying it as a modulating agent.

A typical assay described in Crestey, F. et al., ACS Chem Neurosci (2015), Vol. 6, pp. 1302-1308, AA43279 (Frederiksen, K. et al., Eur J Neurosci (2017), Vol. 46, pp. 1887-1896) and Lu AE98134 (von Schoubyea, N. L. et al., Neurosci Lett (2018), Vol. 662, pp. 29-35) employs the use of automated planar patch clamp techniques to study the effects of the chemical agent on the gating of sodium channels. The sodium channel isoforms of interest are stably expressed in Human Embryonic Kidney Cells and the currents that flow through those channels in response to a depolarizing voltage clamp step from −120 mV to 0 mV are measured in the presence of increasing concentrations of the chemical agents. The area under the sodium current trace which correlates to the magnitude of sodium flux through the cell membrane is used to quantify the effects on gating of the channels. Other parameters that are measured in the assay include the peak current, time constant of open state inactivation and the voltage dependence of steady state inactivation properties. The concentration responses are used to determine potency of each chemical agents effects on modulating the sodium channel isoform gating. Such techniques are known to those skilled in the art, and may be developed, using current technologies, into low or medium throughput assays for evaluating compounds for their ability to modulate sodium channel behaviour. The results of these assays provide the basis for analysis of the structure-activity relationship (SAR) between compounds of the disclosure and the potassium channel. Certain substituents on the core structure of a compound of the disclosure tend to provide more potent inhibitory compounds. SAR analysis is one of the tools those skilled in the art may now employ to identify preferred embodiments of the compounds of the disclosure for use as therapeutic agents.

In an alternative use of the disclosure, the compounds of the disclosure can be used in in vitro or in vivo studies as exemplary agents for comparative purposes to find other compounds also useful in treatment of, or protection from, the various diseases disclosed herein.

In another embodiment, the compounds of Formula (I), as individual stereoisomers, enantiomers or tautomers thereof or mixtures thereof; or pharmaceutically acceptable salts, solvates or prodrugs thereof, as set forth above in the Brief Summary, as stereoisomers, enantiomers, tautomers thereof or mixtures thereof, or pharmaceutically acceptable salts, solvates or prodrugs thereof, and/or the pharmaceutical compositions described herein which comprise a pharmaceutically acceptable excipient and one or more compounds of the disclosure, as set forth above in the Brief Summary, as a stereoisomer, enantiomer or tautomer thereof or mixtures thereof, or a pharmaceutically acceptable salt, solvate or prodrug thereof, can be used in the preparation of a medicament for the treatment of a potassium channel-mediated disease or condition in a mammal.

### Pharmaceutical Compositions and Administration

This disclosure is also directed to pharmaceutical compositions containing the compounds of Formula (I), as described above in the Brief Summary, as stereoisomers, enantiomers, or tautomers thereof or mixtures thereof; or pharmaceutically acceptable salts, solvates or prodrugs thereof. In one embodiment, the present disclosure relates to a pharmaceutical composition comprising compounds of Formula (I), as described above in the Brief Summary, as stereoisomers, enantiomers or tautomers thereof or mixtures thereof; or pharmaceutically acceptable salts, solvates or prodrugs thereof, in a pharmaceutically acceptable carrier, excipient or diluent and in an amount effective to modulate, preferably inhibit, voltage-gated potassium channels to treat certain diseases or conditions, such as epilepsy, when administered to an animal, preferably a mammal, most preferably a human patient.

Administration of the compounds of Formula (I), as described above in the Brief Summary, as stereoisomers, enantiomers, or tautomers thereof or mixtures thereof; or pharmaceutically acceptable salts, solvates or prodrugs thereof, in pure form or in an appropriate pharmaceutical composition, can be carried out via any of the accepted modes of administration of agents for serving similar utilities. The pharmaceutical compositions of the disclosure can be prepared by combining a compound of the disclosure with an appropriate pharmaceutically acceptable carrier, diluent, or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrasternal injection or infusion techniques. Pharmaceutical compositions of the disclosure are formulated to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings of this disclosure.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration.

When intended for oral administration, the pharmaceutical composition is preferably in either solid or liquid form, where semi-solid, semi-liquid, suspension, and gel forms are included within the forms considered herein as either solid or liquid.

As a solid composition for oral administration, the pharmaceutical composition may be formulated into a powder, granule, compressed tablet, pill, capsule, chewing gum, wafer, or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch, and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion, or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer, and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained. Typically, this amount is at least 0.01% of a compound of the disclosure in the composition. When intended for oral administration, this amount may be varied to be between 0.1 and about 70% of the weight of the composition. Preferred oral pharmaceutical compositions contain between about 4% and about 50% of the compound of the disclosure. Preferred pharmaceutical compositions and preparations according to the present disclosure are prepared so that a parenteral dosage unit contains between 0.01 to 10% by weight of the compound prior to dilution.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the disclosure from about 0.1 to about 10% w/v (weight per unit volume).

The pharmaceutical composition of the disclosure may be intended for rectal administration, in the form, for example, of a suppository, which will melt in the rectum and release the drug. The composition for rectal administration may contain an oleaginous base as a suitable non-irritating excipient. Such bases include, without limitation, lanolin, cocoa butter, and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, a protein, or a liposome.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the disclosure may be delivered in single phase, bi-phasic, or tri-phasic systems to deliver the active ingredient(s). Delivery of the aerosol includes the necessary container, activators, valves, sub-containers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining a compound of the disclosure with sterile, distilled water to form a solution. A surfactant may be added to facilitate the formation of a homogeneous solution or suspension. Surfactants are compounds that non-covalently interact with the compound of the disclosure to facilitate dissolution or homogeneous suspension of the compound in the aqueous delivery system.

The compounds of the disclosure, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodmanetna., eds., Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, MD. (1987), Ebadi, Pharmacology, Little, Brown and Co., Boston, (1985); Osolci al., eds., Remington's Pharmaceutical Sciences, 18^{th} edition, Mack Publishing Co., Easton, P A (1990); Katzung, Basic and Clinical Pharmacology, Appleton and Lange, Norwalk, CT (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology or directed to other symptoms of the pathology. The recipients of administration of compounds and/or compositions of the disclosure can be any vertebrate animal, such as mammals. Among mammals, the preferred recipients are mammals of the Orders Primate (including humans, apes, and monkeys), Arteriodactyla (including horses, goats, cows, sheep, pigs), Rodenta (including mice, rats, rabbits, and hamsters), and Carnivora (including cats and dogs). Among birds, the preferred recipients are turkeys, chickens, and other members of the same order. The most preferred recipients are humans.

For topical applications, it is preferred to administer an effective amount of a pharmaceutical composition according to the disclosure to target area, e.g., skin surfaces, mucous membranes, and the like, which are adjacent to peripheral neurons which are to be treated. This amount will generally range from about 0.0001 mg to about 1 g of a compound of the disclosure per application, depending upon the area to be treated, whether the use is diagnostic, prophylactic, or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner, and contact adhesive. Such transdermal patches may be used to provide continuous pulsatile, or on demand delivery of the compounds of the present disclosure as desired.

The compositions of the disclosure can be formulated to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia 22 (6): 543-551 (1997), all of which are incorporated herein by reference.

The compositions of the disclosure can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The disclosure also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern daily, similar to devises used to apply testosterone as described in PCT Published Patent Application No. WO 98/50016.

Current methods for ocular delivery include topical administration (eye drops), subconjunctival injections, periocular injections, intravitreal injections, surgical implants, and iontophoresis (uses a small electrical current to transport ionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (e.g., oral, intravenous, inhalation, sub-cutaneous, rectal etc.), dosage forms, suitable pharmaceutical excipients, and other matters relevant to the delivery of the compounds to a subject in need thereof.

### Combination Therapy

The compounds of the disclosure may be usefully combined with one or more other compounds of the disclosure or one or more other therapeutic agent or as any combination thereof, in the treatment of potassium channel-mediated diseases and conditions. For example, a compound of this disclosure may be administered simultaneously, sequentially, or separately in combination with other therapeutic agents, including, but not limited to: Acetazolamide (Diamox), Brivaracetam (Briviact), Cannabidiol (Epidiolex), Carbamazepine (Tegretol), Cenobamate (Xcopri), Clobazam (Frisium), Clonazepam (Klonopin), Eslicarbazepine acetate (Aptiom, Zebinix), Ethosuximide (Zarontin), Felbamate (Felbatol), Fenfluramine (Fintepla), Gabapentin (Neurontin), Lacosamide (Vimpat), Lamotrigine (Lamictal), Levetiracetam (Keppra), Oxcarbazepine (Trileptal), Perampanel (Fycompa), Phenobarbital (Luminal), Phenytoin (Dilantin), Pregabalin (Lyrica), Primidone, Retigabine (Ezogabine), Rufinamide (Banzel), Stiripentol (Diacomit), Sulthiame, Tiagabine (Gabitril), Topiramate (Topamax), Valproate (Depakote), Vigabatrin (Sabril), Zonisamide (Zonegran).

As used herein “combination” refers to any mixture or permutation of one or more compounds of the disclosure and one or more other compounds of the disclosure or one or more additional therapeutic agent. Unless the context makes clear otherwise, “combination” may include simultaneous or sequentially delivery of a compound of the disclosure with one or more therapeutic agents. Unless the context makes clear otherwise, “combination” may include dosage forms of a compound of the disclosure with another therapeutic agent. Unless the context makes clear otherwise, “combination” may include routes of administration of a compound of the disclosure with another therapeutic agent. Unless the context makes clear otherwise, “combination” may include formulations of a compound of the disclosure with another therapeutic agent. Dosage forms, routes of administration and pharmaceutical compositions include, but are not limited to, those described herein.

### Kits-of-Parts

The present disclosure also provides kits that contain a pharmaceutical composition which includes one or more compounds of the disclosure. The kit also includes instructions for the use of the pharmaceutical composition for modulating the activity of potassium channels, for the treatment of a seizure disorder, such as epilepsy, as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Preparation of Compounds

The following Reaction Schemes illustrate methods to make compounds of the disclosure, i.e., compounds of Formula (I), as described above in the Brief Summary, as stereoisomers, enantiomers, or tautomers thereof or mixtures thereof; or pharmaceutically acceptable salts, solvates or prodrugs thereof.

It is also understood that one skilled in the art would be able to make the compounds of the disclosure by similar methods or by methods known to one skilled in the art. It is also understood that one skilled in the art would be able to make in a similar manner as described below other compounds of the disclosure not specifically illustrated below by using the appropriate starting components and modifying the parameters of the synthesis as needed. In general, starting components may be obtained from sources such as Sigma Aldrich, Alfa Aesar, Combi-Blocks, Oakwood Chemicals, Matrix Scientific, and TCI, etc. or synthesized according to sources known to those skilled in the art (see, e.g., M. B. Smith and J. March, Advanced Organic Chemistry: Reactions, Mechanisms, and Structure, 6th edition (Wiley, 2007)) or prepared as described herein.

It is also understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, include t-butoxycarbonyl, benzyloxycarbonyl, p-methoxybenzyl, trityl, and the like.

Protecting groups may be added or removed in accordance with standard techniques, which are known to one skilled in the art and as described herein.

The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” All prodrugs of compounds of this disclosure are included within the scope of the disclosure.

The compounds of Formula (I) may contain at least one asymmetric carbon atom and thus can exist as racemates, enantiomers and/or diastereoisomers. Specific enantiomers or diastereoisomers may be prepared by utilizing the appropriate chiral starting material. Alternatively, diastereoisomeric mixtures or racemic mixtures of compounds of Formula (I) may be resolved into their respective enantiomers or diastereoisomers. Methods for resolution of diastereoisomeric mixtures or racemic mixtures of the compounds of Formula (I), as described herein, or intermediates prepared herein, are well known in the art (e.g., E. L. Eliel and S. H. Wilen, in Stereochemistry of Organic Compounds; John Wiley & Sons: New York, 1994; Chapter 7, and references cited therein). Suitable processes such as crystallization (e.g., preferential crystallization, preferential crystallization in the presence of additives), asymmetric transformation of racemates, chemical separation (e.g., formation and separation of diastereomers such as diastereomeric salt mixtures or the use of other resolving agents; separation via complexes and inclusion compounds), kinetic resolution (e.g., with titanium tartrate catalyst), enzymatic resolution (e.g., lipase mediated) and chromatographic separation (e.g., HPLC with chiral stationary phase and/or with simulated moving bed technology, or supercritical fluid chromatography and related techniques) are some of the examples that may be applied (see e.g., T. J. Ward, Analytical Chemistry, 2002, 2863-2872).

In general, compounds of Formula (I), as described above in the Brief Summary, can be synthesized following the general procedure described below in Reaction Scheme 1 and 2 wherein X is a halogen (e.g., fluoro, chloro, bromo) and [symbol] , m, n, Y, R¹, R², R³, and R⁴ are as defined above in the Brief Summary and throughout this disclosure.

In some embodiments, one R³ is added as a substituent of [symbol] using suitable reaction conditions, for example, a reaction of —OH with an appropriate reagent (e.g., 2-propanol, cyclopropylmethanol, 1-propanol, 2-methoxyethan-1-ol, pyridin-2-ylmethanol, (5,5-dimethyltetrahydrofuran-2-yl)methanol) using DIAD, PPh₃ in THF as the reaction is warmed from 0° C. to room temperature. In another embodiment, one R³ is added as a substituent of [symbol] using suitable reaction conditions, for example, a reaction of —OH with ethyl-2-bromo-2,2difluoroacetate using K₂CO₃ in DMF as the reaction is warmed from room temperature to 100° C. In some embodiments, one R³ is added as a substituent of [symbol] using suitable reaction conditions, for example, a reaction of —OH with (1-(trifluoromethyl)cyclopropyl)methanol or 1-cyclopropylethan-1-ol using DIAD, PPh₃ in THF as the reaction is warmed from 0° C. to room temperature. In some embodiments, one R³ is modified to afford a substituent of [symbol] using suitable reaction conditions, for example, a reaction that converts —OCH₃ to —OH using suitable reaction conditions (e.g., boron tribromide in dichloromethane at 15° C.).

In another embodiment, one R³ is added as a substituent of [symbol] using suitable reaction conditions, for example, a reaction of —Br with phenol using K₂CO₃, CuI, and picolinic acid in DMSO at 90° C. for 48 hours. In another embodiment, one R³ is added as a substituent of [symbol] using suitable reaction conditions, for example, a reaction of —Br with cyclopropylmethanamine using t-BuXPhos-Pd-G3 and t-BuONa in 1,4,-dioxane at 90° C. for 12 hours. In another embodiment, one R³ is added as a substituent of [symbol] using suitable reaction conditions, for example, a reaction of —Br with iminodimethyl-λ⁶-sulfanone using Xantphos, Pd₂(dba)₃, t-BuONa in 1,4-dioxane at 100° C. for 48 hours.

In some embodiments, X is installed under suitable reaction conditions, for example, by treating starting material (e.g., 6-phenoxyisoquinoline or isoquinoline-6-carboxylate) with mCPBA in dichloromethane followed by a reaction with POCl₃ while heating to 80° C. or 110° C.

In some other embodiments, X is installed using a reaction of starting material (e.g., 6-((cyclopropylmethyl)amino)isoquinolin-1(2H)-one) with POCl₃ while heating to 100° C.

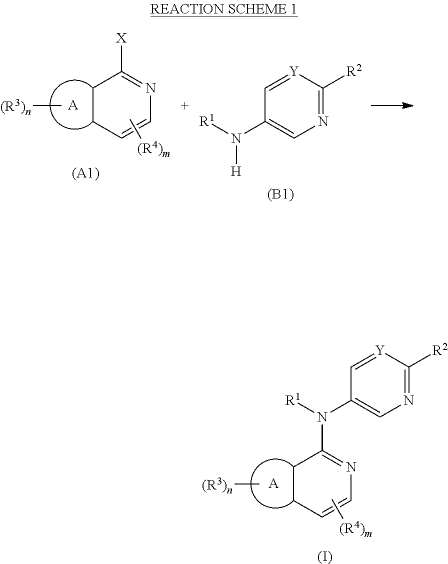

Compounds of formula (A1) and formula (B1) are commercially available or can be prepared by methods known to one skilled in the art or by the methods disclosed herein. In general, compounds of Formula (I) can be prepared by first treating a compound (A1) with compound (1) under suitable reaction conditions (e.g., Pd₂(dba)₃, XPhos, K₃PO₄ in DME or 1,4,-dioxane at 100-110° C. for 4-16 hours, t-BuXPhos-Pd-G3, Cs₂CO₃, in t-amyl alcohol at 25-90° C. for 12 hours, or 4M HCl in 1,4-dioxane and ethanol at 50-85° C.) to afford a compound of Formula (I) as shown.

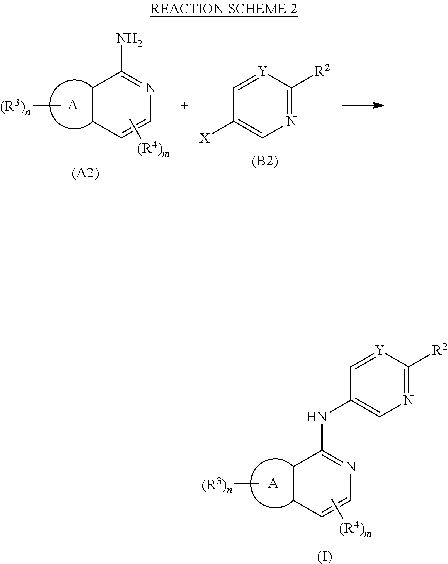

Compounds of formula (A2) and formula (B2) are commercially available or can be prepared by methods known to one skilled in the art or by the methods disclosed herein. In general, compounds of Formula (I) can be prepared by first treating a compound of formula (A2) with a compound of formula (B2) under suitable reaction conditions (e.g., BrettPhos-Pd-G3, t-BuONa in 1,4,-dioxane at 90° C. for 12 hours) to afford a compound of Formula (I) as shown.

All compounds described below as being prepared which may exist in free base or acid form may be converted to their pharmaceutically acceptable salts by treatment with the appropriate inorganic or organic base or acid. Salts of the compounds prepared below may be converted to their free base or acid form by standard techniques. Furthermore, all compounds of the disclosure which contain an acid or an ester group can be converted to the corresponding ester or acid, respectively, by methods known to one skilled in the art or by methods described herein.

The present disclosure also relates to novel intermediate compounds as defined above, all salts, solvates and complexes thereof and all solvates and complexes of salts thereof as defined hereinbefore for compounds of Formula (I). The disclosure includes all polymorphs of the aforementioned species and crystal habits thereof.

The following Examples, which are directed to the synthesis of the compounds of the disclosure; and the following Biological Examples are provided as a guide to assist in the practice of the disclosure and are not intended as a limitation on the scope of the disclosure.

In the Preparations and Examples below, unless otherwise indicated all temperatures are set forth in degrees Celsius. Commercially available reagents were purchased from suppliers such as Sigma Aldrich, Alfa Aesar, Combi-Blocks, Oakwood Chemicals, Matrix Scientific, and TCI, etc. and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried. Yields were not optimized. Melting points were determined on a Büchi hot-stage apparatus and are uncorrected. ¹H NMR, ¹⁹F, and ¹³C NMR data were obtained in deuterated CDCl₃, DMSO-d₆, CD₃OD, CD₃CN, or acetone-d₆ solvent solutions with chemical shifts (b) reported in parts-per-million (ppm) relative to trimethylsilane (TMS) or the residual non-deuterated solvent peaks as the reference standard.

Data are reported as follows, if applicable: chemical shift, multiplicity, coupling constant in Hz, and number of protons, fluorine, or carbon atoms. When peak multiplicities are reported, the following abbreviates are used: s (singlet), d (doublet), t (triplet), q (quartet), m (multiplet), br (broadened), dd (doublet of doublets), dt (doublet of triplets). Coupling constants, when given, are reported in Hz (Hertz).

### Example 1

### Synthesis of N-(6-chloropyridin-3-yl)-6-isopropoxyisoquinolin-1-amine

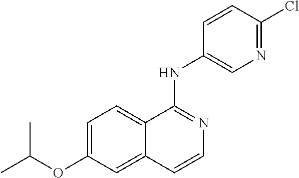 [SMILES: CC(C)Oc1ccc2c(Nc3ccc(Cl)nc3)nccc2c1]

### Step 1. Preparation of 1-chloro-6-isopropoxyisoquinoline

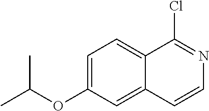

To a solution of 1-chloroisoquinolin-6-ol (0.250 g, 1.39 mmol, prepared according to PCT Published Patent Application No. WO 2012/151195 A1 which is hereby incorporated in its entirety), 2-propanol (0.11 mL, 1.4 mmol), and triphenylphosphine (0.547 g, 2.09 mmol) in anhydrous tetrahydrofuran (7 mL) was added diisopropyl azodicarboxylate (0.41 mL, 2.1 mmol) dropwise at 0° C. with stirring. The reaction mixture was stirred at ambient temperature for 16 h, and concentrated in vacuo. The residue was dissolved in diethyl ether, and heptane was added dropwise with stirring until a pale yellow precipitate was formed. The mixture was triturated, and solid was filtered out. The filtrate was concentrated in vacuo. The residue was re-dissolved in diethyl ether, and a precipitation procedure was repeated twice. The final filtrate was concentrated in vacuo, and the residue was purified by column chromatography, eluting with a gradient of 0 to 20% of ethyl acetate in heptane, to afford the title compound as a colorless solid (0.28 g, 91% yield): MS (ES+) m/z 222.1, 224.1 (M+1).

### Step 2. Preparation of N-(6-chloropyridin-3-yl)-6-isopropoxyisoquinolin-1-amine

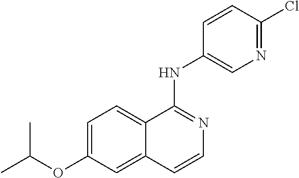 [SMILES: CC(C)Oc1ccc2c(Nc3ccc(Cl)nc3)nccc2c1]

A mixture of 1-chloro-6-isopropoxyisoquinoline (0.270 g, 1.22 mmol), 6-chloropyridin-3-amine (0.157 g, 1.22 mmol), and potassium phosphate tribasic (0.777 g, 3.66 mmol) in 1,2-dimethoxyethane (12 mL) was purged with argon for 20 minutes, and then 2-dicyclohexylphosphino-2′,4′,6′-triisopropylbiphenyl (0.058 g, 0.12 mmol) was added, followed by tris(dibenzylideneacetone)dipalladium(0) (0.056 g, 0.061 mmol). The mixture was purged with argon for additional 5 minutes and then heated to 110° C. for 16 h. The reaction mixture was cooled to ambient temperature, and filtered through a pad of diatomaceous earth. The pad was washed with ethyl acetate (2×20 mL) and the filtrate was concentrated in vacuo. The residue was purified by column chromatography, eluting with a gradient of 0 to 20% of ethyl acetate in heptane, to afford the title compound as a colorless solid (0.083 g, 22% yield): ¹H NMR (300 MHz; DMSO-d₆): δ 9.36 (s, 1H), 8.88 (dd, J=2.8, 0.5 Hz, 1H), 8.45-8.38 (m, 2H), 7.94 (d, J=5.8 Hz, 1H), 7.44 (dd, J=8.7, 0.4 Hz, 1H), 7.30-7.14 (m, 3H), 4.89-4.76 (m, 1H), 1.35 (d, J=6.0 Hz, 6H); MS (ES+) m/z 313.9, 316.0 (M+1).

### Example 2

### Synthesis of N-(6-chloropyridin-3-yl)-6-(cyclopropylmethoxy)isoquinolin-1-amine

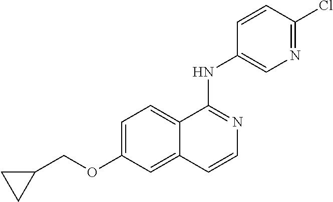 [SMILES: Clc1ccc(Nc2nccc3cc(OCC4CC4)ccc23)cn1]

### Step 1. Preparation of 1-chloro-6-(cyclopropylmethoxy)isoquinoline

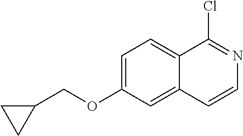 [SMILES: Clc1nccc2cc(OCC3CC3)ccc12]

Following the procedure as described for EXAMPLE 1, Step 1 and making non-critical variations as required to replace 2-propanol with cyclopropylmethanol, the title compound was obtained as a colorless oil which solidified on standing (0.24 g, 84% yield): MS (ES+) m/z 234.1, 236.1 (M+1).

### Step 2. N-(6-chloropyridin-3-yl)-6-(cyclopropylmethoxy)isoquinolin-1-amine

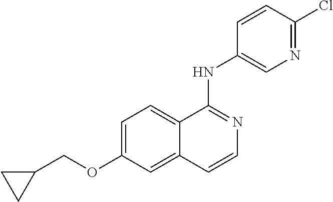 [SMILES: Clc1ccc(Nc2nccc3cc(OCC4CC4)ccc23)cn1]

Following the procedure as described for EXAMPLE 1, Step 2 and making variations as required to replace 1-chloro-6-isopropoxyisoquinoline with 1-chloro-6-(cyclopropylmethoxy)isoquinoline, the title compound was obtained as a colorless solid (0.192 g, 57% yield): ¹H NMR (300 MHz; DMSO-d₆): δ 9.37 (s, 1H), 8.88 (dd, J=2.9, 0.6 Hz, 1H), 8.47-8.37 (m, 2H), 7.95 (d, J=5.8 Hz, 1H), 7.46-7.41 (m, 1H), 7.32-7.22 (m, 2H), 7.16 (d, J=5.5 Hz, 1H), 3.98 (d, J=7.1 Hz, 2H), 1.37-1.20 (m, 1H), 0.65-0.58 (m, 2H), 0.40-0.35 (m, 2H); MS (ES+) m/z 326.0, 328.1 (M+1).

### Example 3

### Synthesis of N-(6-chloropyridin-3-yl)-6-propoxyisoquinolin-1-amine

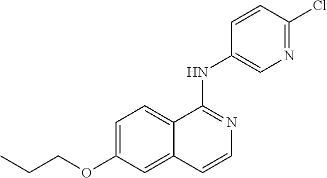 [SMILES: CCCOc1ccc2c(Nc3ccc(Cl)nc3)nccc2c1]

### Step 1. Preparation of 1-chloro-6-propoxyisoquinoline

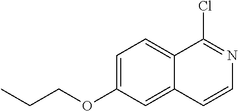

Following the procedure as described for EXAMPLE 1, Step 1 and making variations as required to replace 2-propanol with 1-propanol, the title compound was obtained as a colorless oil (0.22 g, 74% yield): MS (ES+) m/z 222.0, 224.0 (M+1).

### Step 2. Preparation of N-(6-chloropyridin-3-yl)-6-propoxyisoquinolin-1-amine

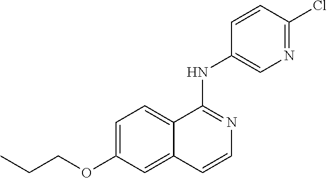 [SMILES: CCCOc1ccc2c(Nc3ccc(Cl)nc3)nccc2c1]

Following the procedure as described for EXAMPLE 1, Step 2 and making variations as required to replace 1-chloro-6-isopropoxyisoquinoline with 1-chloro-6-propoxyisoquinoline, the title compound was obtained as a colorless solid (0.122 g, 42% yield): ¹H NMR (300 MHz; DMSO-d₆): δ 9.37 (s, 1H), 8.88 (dd, J=2.9, 0.5 Hz, 1H), 8.47-8.38 (m, 2H), 7.95 (d, J=5.8 Hz, 1H), 7.44 (dd, J=8.7, 0.4 Hz, 1H), 7.30-7.23 (m, 2H), 7.18 (d, J=5.6 Hz, 1H), 4.08 (t, J=6.6 Hz, 2H), 1.86-1.75 (m, 2H), 1.02 (t, J=7.4 Hz, 3H); MS (ES+) m/z 313.8, 316.0 (M+1).

### Example 4

### Synthesis of N-(6-chloropyridin-3-yl)-6-(2-methoxyethoxy)isoquinolin-1-amine

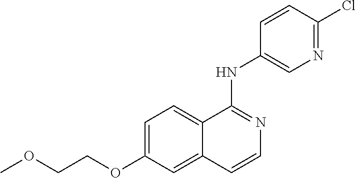 [SMILES: COCCOc1ccc2c(Nc3ccc(Cl)nc3)nccc2c1]

### Step 1. Preparation of 1-chloro-6-(2-methoxyethoxy)isoquinoline

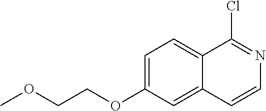 [SMILES: COCCOc1ccc2c(Cl)nccc2c1]

Following the procedure as described for EXAMPLE 1, Step 1 and making variations as required to replace 2-propanol with 2-methoxyethan-1-ol, the title compound was obtained as a colorless solid (0.27 g, 91% yield): MS (ES+) m/z 238.0, 240.0 (M+1).

### Step 2. Preparation of N-(6-chloropyridin-3-yl)-6-(2-methoxyethoxy)isoquinolin-1-amine

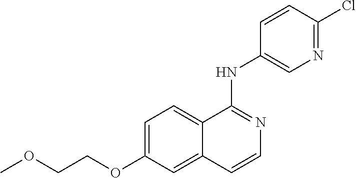 [SMILES: COCCOc1ccc2c(Nc3ccc(Cl)nc3)nccc2c1]

Following the procedure as described for EXAMPLE 1, Step 2 and making variations as required to replace 1-chloro-6-isopropoxyisoquinoline with 1-chloro-6-(2-methoxyethoxy)isoquinoline, the title compound was obtained as a colorless solid (0.13 g, 35% yield): ¹H NMR (300 MHz; DMSO-d₆): δ 9.38 (s, 1H), 8.88 (d, J=2.7 Hz, 1H), 8.48-8.37 (m, 2H), 7.96 (d, J=6.8 Hz, 1H), 7.45 (d, J=8.7 Hz, 1H), 7.34-7.28 (m, 2H), 7.17 (d, J=5.8 Hz, 1H), 4.33-4.19 (m, 2H), 3.80-3.64 (m, 2H), 3.41-3.23 (m, 3H); MS (ES+) m/z 330.0, 331.9 (M+1).

### Example 5

### Synthesis of N-(6-chloropyridin-3-yl)-6-(pyridin-2-ylmethoxy)isoquinolin-1-amine

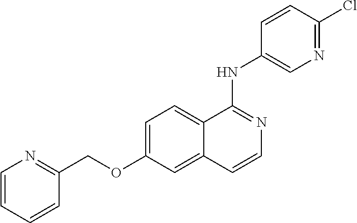 [SMILES: Clc1ccc(Nc2nccc3cc(OCc4ccccn4)ccc23)cn1]

### Step 1. Preparation of 1-chloro-6-(pyridin-2-ylmethoxy)isoquinoline

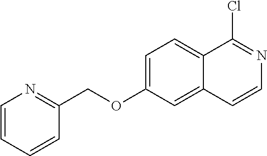 [SMILES: Clc1nccc2cc(OCc3ccccn3)ccc12]

Following the procedure as described for EXAMPLE 1, Step 1 and making variations as required to replace 2-propanol with pyridin-2-ylmethanol, the title compound was obtained as a colorless oil which solidified on standing (0.060 g, 16% yield): MS (ES+) m/z 271.1, 273.1 (M+1).

### Step 2. Preparation of N-(6-chloropyridin-3-yl)-6-(pyridin-2-ylmethoxy)isoquinolin-1-amine

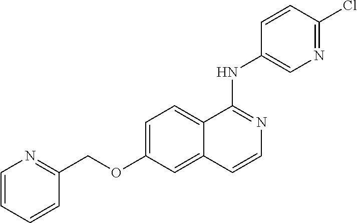 [SMILES: Clc1ccc(Nc2nccc3cc(OCc4ccccn4)ccc23)cn1]

Following the procedure as described for EXAMPLE 1, Step 2 and making variations as required to replace 1-chloro-6-isopropoxyisoquinoline with 1-chloro-6-(pyridin-2-ylmethoxy)isoquinoline, the title compound was obtained as a colorless solid (0.010 g, 13% yield): ¹H NMR (300 MHz; DMSO-d₆): δ 9.40 (s, 1H), 8.88 (d, J=2.6 Hz, 1H), 8.65-8.58 (m, 1H), 8.49-8.39 (m, 2H), 7.96 (d, J=5.8 Hz, 1H), 7.87 (td, J=7.7, 1.8 Hz, 1H), 7.60 (d, J=7.8 Hz, 1H), 7.46-7.35 (m, 4H), 7.18 (d, J=5.8 Hz, 1H), 5.34 (s, 2H); MS (ES+) m/z 363.4, 365.4 (M+1).

### Example 6

### Synthesis of N-(6-chloropyridin-3-yl)-6-((5,5-dimethyltetrahydrofuran-2-yl)methoxy)isoquinolin-1-amine

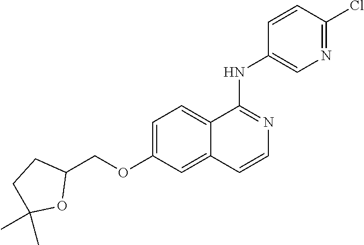 [SMILES: CC1(C)CCC(COc2ccc3c(Nc4ccc(Cl)nc4)nccc3c2)O1]

### Step 1. Preparation of 1-chloro-6-((5,5-dimethyltetrahydrofuran-2-yl)methoxy)isoquinoline

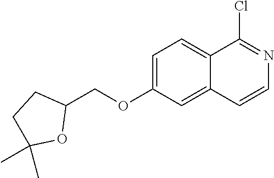 [SMILES: CC1(C)CCC(COc2ccc3c(Cl)nccc3c2)O1]

Following the procedure as described for EXAMPLE 1, Step 1 and making variations as required to replace 2-propanol with (5,5-dimethyltetrahydrofuran-2-yl)methanol, the title compound was obtained as a colorless solid (0.20 g, 54% yield): MS (ES+) m/z 292.0, 294.0 (M+1).

### Step 2. N-(6-chloropyridin-3-yl)-6-((5,5-dimethyltetrahydrofuran-2-yl)methoxy)isoquinolin-1-amine

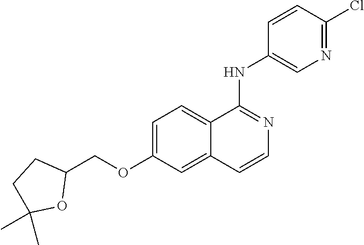 [SMILES: CC1(C)CCC(COc2ccc3c(Nc4ccc(Cl)nc4)nccc3c2)O1]

Following the procedure as described for EXAMPLE 1, Step 2 and making variations as required to replace 1-chloro-6-isopropoxyisoquinoline with 1-chloro-6-((5,5-dimethyltetrahydrofuran-2-yl)methoxy)isoquinoline, the title compound was obtained as a colorless solid (0.098 g, 37% yield): ¹H NMR (300 MHz; DMSO-d₆): δ 9.39 (s, 1H), 8.92-8.82 (m, 1H), 8.50-8.34 (m, 2H), 7.95 (d, J=5.8 Hz, 1H), 7.44 (dd, J=8.7, 0.4 Hz, 1H), 7.33-7.24 (m, 2H), 7.17 (d, J=5.8 Hz, 1H), 4.43-4.20 (m, 1H), 4.16-4.01 (m, 2H), 2.19-2.03 (m, 1H), 1.92-1.68 (m, 3H), 1.29-1.17 (m, 6H); MS (ES+) m/z 384.2, 386.2 (M+1).

### Example 7

### Synthesis of N-(6-chloropyridin-3-yl)-6-(difluoromethoxy)isoquinolin-1-amine

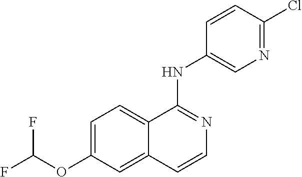 [SMILES: FC(F)Oc1ccc2c(Nc3ccc(Cl)nc3)nccc2c1]

### Step 1. Preparation of 1-chloro-6-(difluoromethoxy)isoquinoline

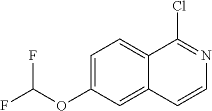 [SMILES: FC(F)Oc1ccc2c(Cl)nccc2c1]

To a stirred solution of 1-chloroisoquinolin-6-ol (0.500 g, 2.78 mmol) in anhydrous N,N-dimethylformamide (20 mL) at ambient temperature was added potassium carbonate (0.576 g, 4.17 mmol), followed by ethyl bromodifluoroacetate (0.43 mL, 3.3 mmol). The reaction mixture was stirred at ambient temperature for 48 h, and at 100° C. for 4.5 h, cooled to ambient temperature, diluted with water (40 mL), saturated aqueous sodium bicarbonate (20 mL), and extracted with ethyl acetate (3×20 mL). The combined organic layers were washed with brine (40 mL), dried over anhydrous magnesium sulfate, filtered, and concentrated in vacuo. The residue was purified by column chromatography, eluting with 0 to 50% of ethyl acetate in heptane, to afford the title compound as a colorless solid (0.205 g, 32% yield): MS (ES+) m/z 230.4, 232.4 (M+1).

### Step 2. Preparation of N-(6-chloropyridin-3-yl)-6-(difluoromethoxy)isoquinolin-1-amine

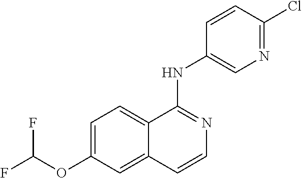 [SMILES: FC(F)Oc1ccc2c(Nc3ccc(Cl)nc3)nccc2c1]

Following the procedure as described for EXAMPLE 1, Step 2 and making variations as required to replace 1-chloro-6-isopropoxyisoquinoline with 1-chloro-6-(difluoromethoxy)isoquinoline, the title compound was obtained as a colorless solid (0.095 g, 34% yield): ¹H NMR (300 MHz; DMSO-d₆): δ 9.53 (s, 1H), 8.88 (dd, J=2.8, 0.5 Hz, 1H), 8.60 (d, J=9.3 Hz, 1H), 8.42 (dd, J=8.8, 2.9 Hz, 1H), 8.04 (d, J=5.8 Hz, 1H), 7.75-7.23 (m, 5H); MS (ES+) m/z 322.0, 323.9 (M+1)

### Example 8

### Synthesis of N-(6-chloropyridin-3-yl)-6-phenoxyisoquinolin-1-amine

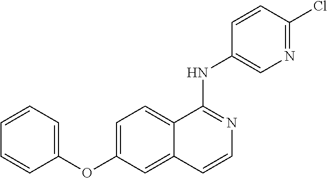 [SMILES: Clc1ccc(Nc2nccc3cc(Oc4ccccc4)ccc23)cn1]

### Step 1. Preparation of 6-phenoxyisoquinoline

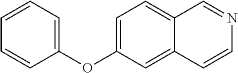 [SMILES: c1ccc(Oc2ccc3cnccc3c2)cc1]

A mixture of 6-bromoisoquinoline (0.600 g, 2.88 mmol), phenol (0.299 g, 3.17 mmol), copper(I) iodide (0.122 g, 0.580 mmol), potassium carbonate (1.83 g, 8.64 mmol), and picolinic acid (0.144 g, 1.15 mmol) in anhydrous dimethyl sulfoxide (15 mL) was purged with nitrogen for 10 minutes and heated to 90° C. with stirring for 48 h. The mixture was cooled to ambient temperature, diluted with water, and extracted with ethyl acetate (3×20 mL). The combined organic layers were washed with saturated aqueous sodium bicarbonate (30 mL), brine (40 mL), dried over anhydrous magnesium sulfate, filtered, and concentrated in vacuo. The residue was purified by column chromatography, eluting with a gradient of 0 to 10% of methanol in dichloromethane, to afford the title compound as a pale yellow oil (0.54 g, 85% yield): MS (ES+) m/z 222.1 (M+1).

### Step 2. Preparation of 6-phenoxyisoquinoline 2-oxide

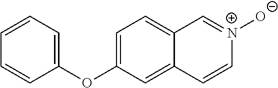 [SMILES: [O-][n+]1ccc2cc(Oc3ccccc3)ccc2c1]

To a stirred solution of 6-phenoxyisoquinoline (0.540 g, 2.44 mmol) in anhydrous dichloromethane (20 mL) at 0° C. was added 3-chloroperoxybenzoic acid (0.840 g, 4.88 mmol) in portions. The reaction mixture was stirred at ambient temperature for 16 h, and diluted with dichloromethane until clear solution was obtained. A 1 M aqueous sodium hydroxide solution was added, and the mixture was diluted further with water, dichloromethane, and chloroform.

The layers were separated, and the organic layer was washed with a 1 M aqueous sodium hydroxide solution (1×30 mL), brine (40 mL), dried over anhydrous magnesium sulfate, filtered, and concentrated in vacuo to afford the title compound as a pale brown solid (0.49 g, 85% yield): MS (ES+) m/z 238.0 (M+1).

### Step 3. Preparation of 1-chloro-6-phenoxyisoquinoline

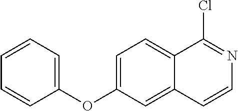 [SMILES: Clc1nccc2cc(Oc3ccccc3)ccc12]

A mixture of 6-phenoxyisoquinoline 2-oxide (0.490 g, 2.07 mmol) and phosphorus (V) oxychloride (3.85 mL, 41.3 mmol) was heated to 110° C. with stirring for 4.5 h. The reaction mixture was cooled to ambient temperature and concentrated in vacuo. The residue was cooled to 0° C., diluted with an ice-water and dichloromethane, and a 2 N aqueous potassium hydroxide solution was added slowly with stirring until the aqueous layer was made alkaline. The mixture was stirred at ambient temperature for 10 minutes, layers were separated, and the aqueous layer was extracted with dichloromethane (2×50 mL). The combined organic layers were washed with brine (1×60 mL), dried over anhydrous magnesium sulfate, filtered, and concentrated in vacuo. The residue was purified by column chromatography, eluting with a gradient of 0 to 30% of ethyl acetate in heptane, to afford the title compound as a colorless oil (0.325 g, 61% yield): MS (ES+) m/z 256.0, 258.0 (M+1).

### Step 4. Preparation of N-(6-chloropyridin-3-yl)-6-phenoxyisoquinolin-1-amine

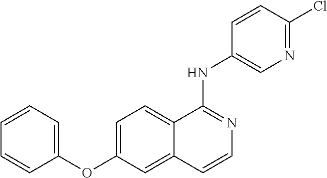 [SMILES: Clc1ccc(Nc2nccc3cc(Oc4ccccc4)ccc23)cn1]

Following the procedure as described for EXAMPLE 1, Step 2 and making variations as required to replace 1-chloro-6-isopropoxyisoquinoline with 1-chloro-6-phenoxyisoquinoline, the title compound was obtained as a colorless solid (0.070 g, 17% yield): ¹H NMR (300 MHz; DMSO-d₆): δ 9.48 (s, 1H), 8.88-8.87 (m, 1H), 8.56 (d, J=9.3 Hz, 1H), 8.42 (dd, J=8.8, 2.9 Hz, 1H), 7.96 (d, J=5.8 Hz, 1H), 7.52-7.40 (m, 4H), 7.29-7.14 (m, 5H); MS (ES+) m/z 348.0, 350.0 (M+1).

### Example 9

### Synthesis of N-(2-chloropyrimidin-5-yl)-6-(cyclopropylmethoxy)isoquinolin-1-amine

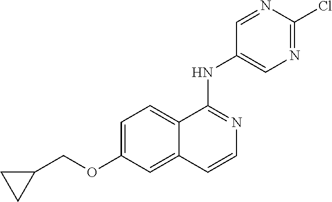 [SMILES: Clc1ncc(Nc2nccc3cc(OCC4CC4)ccc23)cn1]

A mixture of 1-chloro-6-(cyclopropylmethoxy)isoquinoline (0.200 g, 0.856 mmol), 2-chloropyrimidin-5-amine (0.111 g, 0.856 mmol), and potassium phosphate tribasic (0.550 g, 2.57 mmol) in 1,2-dimethoxyethane (9 mL) was purged with argon for 20 minutes, and then 2-dicyclohexylphosphino-2′,4′,6′-triisopropylbiphenyl (0.041 g, 0.086 mmol) was added, followed by tris(dibenzylideneacetone)dipalladium(0) (0.039 g, 0.043 mmol). The mixture was purged with argon for additional 5 minutes and then heated to 110° C. for 16 h. The reaction mixture was cooled to ambient temperature, and filtered through a pad of diatomaceous earth. The pad was washed with ethyl acetate (2×20 mL), and the filtrate was concentrated in vacuo. The residue was purified by column chromatography, eluting with a gradient of 0 to 30% of ethyl acetate in heptane, to afford the title compound as a colorless solid (0.076 g, 27% yield): ¹H NMR (300 MHz; DMSO-d₆): δ 9.56 (s, 1H), 9.29 (s, 2H), 8.41 (d, J=9.2 Hz, 1H), 7.98 (d, J=5.8 Hz, 1H), 7.35-7.21 (m, 3H), 3.98 (d, J=7.1 Hz, 2H), 1.34-1.25 (m, 1H), 0.65-0.59 (m, 2H), 0.40-0.35 (m, 2H); MS (ES+) m/z 327.0, 329.0 (M+1).

### Example 10

### Synthesis of methyl 1-((6-chloropyridin-3-yl)amino)isoquinoline-6-carboxylate

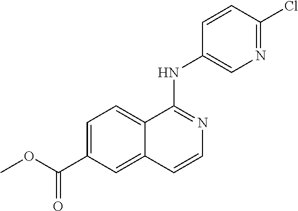 [SMILES: COC(=O)c1ccc2c(Nc3ccc(Cl)nc3)nccc2c1]

### Step 1. Preparation of 6-(methoxycarbonyl)isoquinoline 2-oxide

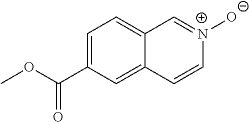

To a stirred solution of methyl isoquinoline-6-carboxylate (4.05 g, 21.6 mmol) in dichloromethane (160 mL) at 0° C. was added 3-chloroperoxybenzoic acid (9.70 g, 43.3 mmol) in portions. The reaction mixture was warmed to ambient temperature, stirred for 4 h, cooled to 0° C., and a 1 M aqueous sodium hydroxide solution was added (35 mL). The reaction mixture was diluted with saturated aqueous sodium bicarbonate and brine. The layers were separated and the organics were washed with saturated aqueous sodium bicarbonate (4×30 mL), dried over anhydrous magnesium sulfate, filtered and concentrated in vacuo. The residue was triturated in hexanes (100 mL), the solid was filtered out, washed with hexanes (2×10 mL), and dried to afford the title compound as a brown solid (4.19 g, 95% yield). The product was used in the next step without further purification: LCMS (ES+) m/z 204.4 (M+1)

### Step 2. Preparation of methyl 1-chloroisoquinoline-6-carboxylate

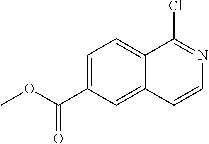

A mixture of 6-(methoxycarbonyl)isoquinoline 2-oxide (1.36 g, 6.69 mmol) and phosphorus (V) oxychloride (5.4 mL, 58 mmol) was stirred at 80° C. for 3 h. The reaction mixture was cooled to 0° C. and water (15 mL) was added dropwise with stirring. The precipitate was filtered out, washed with cold water (2×10 mL) and purified by column chromatography, eluting with a gradient of 0 to 20% of ethyl acetate in heptane, to afford the title compound as a yellow solid (0.49 g, 33% yield): ¹H-NMR (300 MHz CDCl₃): δ 8.62-8.56 (m, 1H), 8.44-8.34 (m, 2H), 8.30-8.24 (m, 1H), 7.77-7.70 (m, 1H), 4.02 (s, 3H); LCMS (ES+) m/z 222.6, 224.6 (M+1).

### Step 3. Preparation of methyl 1-((6-chloropyridin-3-yl)amino)isoquinoline-6-carboxylate

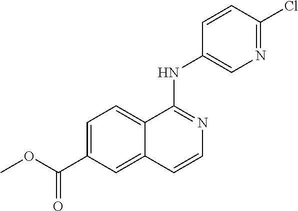 [SMILES: COC(=O)c1ccc2c(Nc3ccc(Cl)nc3)nccc2c1]

Following the procedure as described for EXAMPLE 1, Step 2 and making variations as required to replace 1-chloro-6-isopropoxyisoquinoline with methyl 1-chloroisoquinoline-6-carboxylate, the title compound was obtained as a yellow solid (0.233 mg, 34% yield): ¹H NMR (300 MHz; DMSO-d₆): δ 9.64 (s, 1H), 8.93-8.87 (m, 1H), 8.64 (d, J=8.9 Hz, 1H), 8.53 (d, J=1.7 Hz, 1H), 8.47-8.39 (m, 1H), 8.18-8.06 (m, 2H), 7.55-7.42 (m, 2H), 3.94 (s, 3H); LCMS (ES+) m/z 314.6, 316.6 (M+1)

### Example 11

### Synthesis of N-(6-chloropyridin-3-yl)thieno[3,2-c]pyridin-4-amine formate

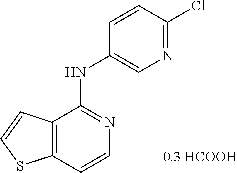 [SMILES: Clc1ccc(Nc2nccc3sccc23)cn1]

To a mixture of 4-chlorothieno[3,2-c]pyridine (0.0470 g, 0.277 mmol), 6-chloropyridin-3-amine (0.356 g, 0.277 mmol) and cesium carbonate (0.271 g, 0.831 mmol) in tert-amyl alcohol (1 mL) was added [(2-di-tert-butylphosphino-2′,4′,6′-triisopropyl-1,1′-biphenyl)-2-(2′-amino-1,1′-biphenyl)] palladium(II) methanesulfonate (0.0220 g, 0.0277 mmol) in glove box. The mixture was heated to 80° C. and stirred for 12 h. The mixture was cooled to ambient temperature, diluted with ethyl acetate (5 mL) and thiourea resin (0.100 g) was added. The mixture was stirred at 25° C. for 4 h, and filtered. The filtrate was concentrated in vacuo. The residue was purified by preparative reverse-phase HPLC, using acetonitrile in water containing 0.225% of formic acid as eluent, to afford the title compound as a yellow solid (0.0342 g, 40% yield): ¹H NMR (400 MHz, CD₃OD) δ 8.80 (d, J=2.8 Hz, 1H), 8.51 (s, 0.3H), 8.36 (dd, J=8.8, 2.8 Hz, 1H), 7.98 (d, J=5.6 Hz, 1H), 7.81 (d, J=5.6 Hz, 1H), 7.63 (d, J=5.6 Hz, 1H), 7.43 (d, J=5.6 Hz, 1H), 7.39 (d, J=8.8 Hz, 1H), exchangeable protons not observed; MS (ES+) m/z 262.2, 264.2 (M+1).

### Example 12

### Synthesis of N-(6-chloropyridin-3-yl)thieno[2,3-c]pyridin-7-amine

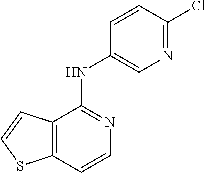 [SMILES: Clc1ccc(Nc2nccc3sccc23)cn1]

Following the procedure as described for EXAMPLE 11 and making variations as required to replace 4-chlorothieno[3,2-c]pyridine with 7-chlorothieno[2,3-c]pyridine, the title compound was obtained as a yellow solid (0.0474 g, 38% yield): ¹H NMR (400 MHz; CDCl₃): δ 8.51 (d, J=2.8 Hz, 1H), 8.30 (dd, J=8.7, 2.9 Hz, 1H), 8.14 (d, J=5.5 Hz, 1H), 7.65 (d, J=5.3 Hz, 1H), 7.39 (d, J=5.3 Hz, 1H), 7.35-7.29 (m, 2H), 6.51 (s, 1H); MS (ES+) m/z 262.0, 264.0 (M+1).

### Example 13

### Synthesis of N-(6-chloropyridin-3-yl)thiazolo[4,5-c]pyridin-4-amine

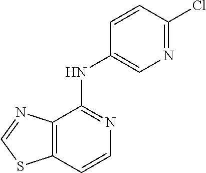 [SMILES: Clc1ccc(Nc2nccc3scnc23)cn1]

Following the procedure as described for EXAMPLE 11 and making variations as required to replace 4-chlorothieno[3,2-c]pyridine with 4-chlorothiazolo[4,5-c]pyridine, the title compound was obtained as a yellow solid (0.0441 g, 41% yield): ¹H NMR (400 MHz; CDCl₃): δ 8.92 (s, 1H), 8.70-8.67 (m, 1H), 8.58 (dd, J=8.7, 2.9 Hz, 1H), 8.17 (d, J=5.7 Hz, 1H), 8.00 (s, 1H), 7.38 (d, J=5.7 Hz, 1H), 7.33 (d, J=8.7 Hz, 1H); MS (ES+) m/z 263.0, 265.0 (M+1).

### Example 14

### Synthesis of N-(6-chloropyridin-3-yl)-1H-pyrrolo[2,3-c]pyridin-7-amine

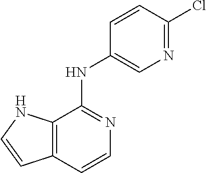 [SMILES: Clc1ccc(Nc2nccc3cc[nH]c23)cn1]

Following the procedure as described for EXAMPLE 11 and making variations as required to replace 4-chlorothieno[3,2-c]pyridine with 7-chloro-1H-pyrrolo[2,3-c]pyridine, the title compound was obtained as a yellow solid (0.0136 g, 21% yield): ¹H NMR (400 MHz; DMSO-d₆): δ 11.32 (s, 1H), 9.15 (s, 1H), 8.79 (d, J=2.6 Hz, 1H), 8.48 (dd, J=8.7, 2.9 Hz, 1H), 7.77-7.68 (m, 1H), 7.59-7.53 (m, 1H), 7.43 (d, J=8.7 Hz, 1H), 7.10 (d, J=5.6 Hz, 1H), 6.49-6.44 (m, 1H); MS (ES+) m/z 245.1, 247.1 (M+1).

### Example 15

### Synthesis of N-(6-chloropyridin-3-yl)-4-methoxy-1H-pyrrolo[2,3-c]pyridin-7-amine formate

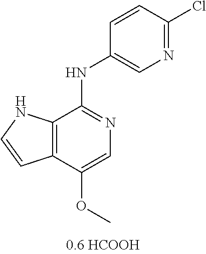 [SMILES: COc1cnc(Nc2ccc(Cl)nc2)c2[nH]ccc12]

Following the procedure as described for EXAMPLE 11 and making variations as required to replace 4-chlorothieno[3,2-c]pyridine with 7-chloro-4-methoxy-1H-pyrrolo[2,3-c]pyridine, the title compound was obtained as a colorless solid (0.0184 g, 20% yield): ¹H NMR (400 MHz; CD₃OD): δ 8.52-8.51 (m, 1H), 8.20 (s, 0.6H), 8.07-8.03 (m, 1H), 7.40 (d, J=3.0 Hz, 1H), 7.35-7.32 (m, 2H), 6.61 (d, J=3.0 Hz, 1H), 3.97 (s, 3H), exchangeable protons not observed; MS (ES+) m/z 275.2, 277.2 (M+1).

### Example 16

### Synthesis of N-(6-chloropyridin-3-yl)furo[2,3-c]pyridin-7-amine formate

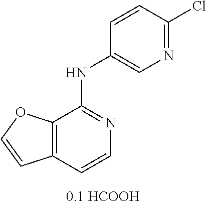 [SMILES: Clc1ccc(Nc2nccc3ccoc23)cn1]

Following the procedure as described for EXAMPLE 11 and making variations as required to replace 4-chlorothieno[3,2-c]pyridine with 7-chlorofuro[2,3-c]pyridine, the title compound was obtained as a yellow solid (0.0492 g, 34% yield): ¹H NMR (400 MHz; CD₃OD): δ 8.81 (d, J=2.9 Hz, 1H), 8.51 (s, 0.1H), 8.36 (dd, J=8.8, 2.9 Hz, 1H), 7.96-7.92 (m, 2H), 7.37 (d, J=8.7 Hz, 1H), 7.16 (d, J=5.5 Hz, 1H), 6.91 (d, J=2.1 Hz, 1H), exchangeable protons not observed; MS (ES+) m/z 246.2, 248.2 (M+1).

### Example 17

### Synthesis of N-(6-chloropyridin-3-yl)-3-methylthieno[2,3-c]pyridin-7-amine

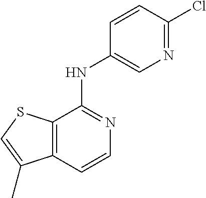 [SMILES: Cc1csc2c(Nc3ccc(Cl)nc3)nccc12]

Following the procedure as described for EXAMPLE 11 and making variations as required to replace 4-chlorothieno[3,2-c]pyridine with 7-chloro-3-methylthieno[2,3-c]pyridine, the title compound was obtained as a grey solid (0.0515 g, 41% yield): ¹H-NMR (400 MHz; CDCl₃): δ ¹H NMR (400 MHz; CDCl₃): δ 8.52 (d, J=2.9 Hz, 1H), 8.30 (dd, J=8.7, 2.9 Hz, 1H), 8.17 (d, J=5.6 Hz, 1H), 7.31 (d, J=8.7 Hz, 1H), 7.27-7.26 (m, 1H), 7.23 (d, J=5.6 Hz, 1H), 6.52 (br s, 1H), 2.46 (d, J=1.1 Hz, 3H); MS (ES+) m/z 276.0, 278.0 (M+1).

### Example 18

### Synthesis of N-(6-chloropyridin-3-yl)-6-fluoroisoquinolin-1-amine formate

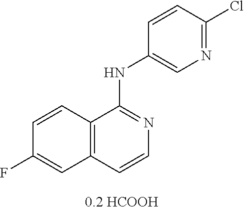 [SMILES: Fc1ccc2c(Nc3ccc(Cl)nc3)nccc2c1]

Following the procedure as described for EXAMPLE 11 and making variations as required to replace 4-chlorothieno[3,2-c]pyridine with 1-chloro-6-fluoroisoquinoline, the title compound was obtained as a yellow solid (0.0361 g, 39% yield): ¹H NMR (400 MHz; CD₃OD): δ 8.76 (d, J=2.6 Hz, 1H), 8.51 (s, 0.2H), 8.45-8.40 (m, 1H), 8.33-8.27 (m, 1H), 7.98 (d, J=5.9 Hz, 1H), 7.51-7.46 (m, 1H), 7.44-7.37 (m, 2H), 7.21 (d, J=5.8 Hz, 1H), exchangeable protons not observed; MS (ES+) m/z 274.1, 276.1 (M+1).

### Example 19

### Synthesis of N-(6-chloropyridin-3-yl)-6-methoxyisoquinolin-1-amine formate

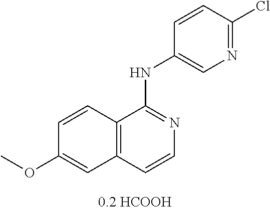 [SMILES: COc1ccc2c(Nc3ccc(Cl)nc3)nccc2c1]

Following the procedure as described for EXAMPLE 11 and making variations as required to replace 4-chlorothieno[3,2-c]pyridine with 1-chloro-6-methoxyisoquinoline, the title compound was obtained as a yellow solid (0.0324 g, 35% yield): ¹H NMR (400 MHz; CD₃OD): δ 8.73-8.70 (m, 1H), 8.49 (s, 0.2H), 8.27-8.22 (m, 2H), 7.90 (d, J=5.9 Hz, 1H), 7.41-7.37 (m, 1H), 7.24-7.16 (m, 3H), 3.95 (s, 3H), exchangeable protons not observed; MS (ES+) m/z 286.0, 288.0 (M+1).

### Example 20

### Synthesis of N-(6-chloropyridin-3-yl)-6-methylisoquinolin-1-amine formate

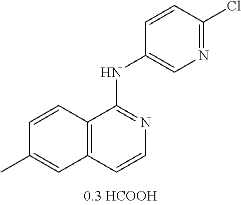 [SMILES: Cc1ccc2c(Nc3ccc(Cl)nc3)nccc2c1]

Following the procedure as described for EXAMPLE 11 and making variations as required to replace 4-chlorothieno[3,2-c]pyridine with 1-chloro-6-methylisoquinoline, the title compound was obtained as a colorless solid (0.0392 g, 42% yield): ¹H NMR (400 MHz; CD₃OD): δ 8.78-8.76 (m, 1H), 8.48 (s, 0.3), 8.33-8.29 (m, 1H), 8.27-8.24 (m, 1H), 7.95 (d, J=5.8 Hz, 1H), 7.62 (s, 1H), 7.51-7.47 (m, 1H), 7.44-7.40 (m, 1H), 7.18-7.17 (m, 1H), 2.55 (s, 3H), exchangeable protons not observed; MS (ES+) m/z 270.0, 272.0 (M+1).

### Example 21

### Synthesis of 6-chloro-N-(6-chloropyridin-3-yl)isoquinolin-1-amine

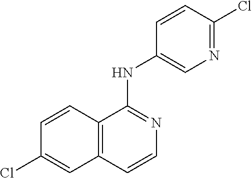 [SMILES: Clc1ccc2c(Nc3ccc(Cl)nc3)nccc2c1]

Following the procedure as described for EXAMPLE 11 and making variations as required to replace 4-chlorothieno[3,2-c]pyridine with 1,6-dichloroisoquinoline, the title compound was obtained as a yellow solid (0.0285 g, 37% yield): ¹H NMR (400 MHz; CDCl₃): δ 8.54 (d, J=2.8 Hz, 1H), 8.39-8.33 (m, 1H), 8.10 (d, J=5.9 Hz, 1H), 7.89 (d, J=9.0 Hz, 1H), 7.78 (d, J=2.1 Hz, 1H), 7.56-7.51 (m, 1H), 7.36-7.31 (m, 1H), 7.16-7.06 (m, 2H); MS (ES+) m/z 290.1, 292.1 (M+1).

### Example 22

### Synthesis of N-(6-chloropyridin-3-yl)-5-fluoroisoquinolin-1-amine

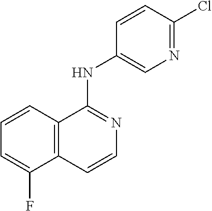 [SMILES: Fc1cccc2c(Nc3ccc(Cl)nc3)nccc12]

Following the procedure as described for EXAMPLE 11 and making variations as required to replace 4-chlorothieno[3,2-c]pyridine with 1-chloro-5-fluoroisoquinoline, the title compound was obtained as a colorless solid (0.0198 g, 26% yield): ¹H NMR (400 MHz; CDCl₃): δ 8.55 (d, J=2.9 Hz, 1H), 8.44-8.37 (m, 1H), 8.15 (d, J=5.8 Hz, 1H), 7.73 (d, J=8.5 Hz, 1H), 7.58-7.50 (m, 1H), 7.44 (d, J=5.8 Hz, 1H), 7.40-7.32 (m, 2H), 7.14 (s, 1H); MS (ES+) m/z 274.1, 264.1 (M+1).

### Example 23

### Synthesis of N-(2-chloropyrimidin-5-yl)-6-fluoroisoquinolin-1-amine

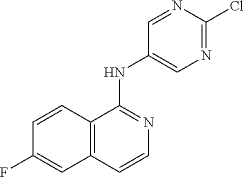 [SMILES: Fc1ccc2c(Nc3cnc(Cl)nc3)nccc2c1]

To a mixture of 1-chloro-6-fluoroisoquinoline (0.315 g, 1.73 mmol), 2-chloropyrimidin-5-amine (0.225 g, 1.73 mmol) and cesium carbonate (1.70 g, 5.20 mmol) in tert-amyl alcohol (6 mL) was added [(2-di-tert-butylphosphino-2′,4′,6′-triisopropyl-1,1′-biphenyl)-2-(2′-amino-1,1′-biphenyl)] palladium(II) methanesulfonate (0.138 g, 0.173 mmol) in glove box. The mixture was stirred at 70° C. for 12 h, cooled to ambient temperature, diluted with ethyl acetate (20 mL), and thiourea resin (1.00 g) was added. The mixture was stirred at 25° C. for 4 h and filtered. The filtrate was concentrated in vacuo. Purification of the residue by preparative HPLC, eluting with ethanol containing 0.1% of ammonium hydroxide in heptane, followed by preparative reverse-phase HPLC, eluting with acetonitrile in water containing 0.225% of formic acid, afforded the title compound as a colorless solid (0.0909 g, 19% yield): ¹H NMR (400 MHz, DMSO-d₆) δ 9.23 (s, 2H), 8.45 (dd, J=5.2, 9.2 Hz, 1H), 8.06 (d, J=6.0 Hz, 1H), 7.53 (dd, J=2.8, 9.6 Hz, 1H), 7.44 (dt, J=2.4, 8.8 Hz, 1H), 7.28 (d, J=5.6 Hz, 1H), exchangeable proton not observed; MS (ES+) m/z 275.1, 277.1 (M+1).

### Example 24

### Synthesis of 6-(cyclopropylmethoxy)-N-(2-methylpyrimidin-5-yl)isoquinolin-1-amine

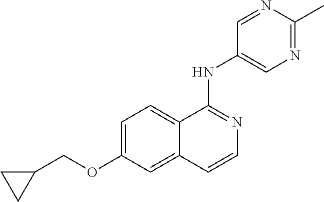 [SMILES: Cc1ncc(Nc2nccc3cc(OCC4CC4)ccc23)cn1]

To a mixture of 1-chloro-6-(cyclopropylmethoxy)isoquinoline (0.500 g, 2.14 mmol), 2-methylpyrimidin-5-amine (0.222 g, 2.03 mmol) and cesium carbonate (2.09 g, 6.42 mmol) in tert-amyl alcohol (10 mL) was added [(2-di-tert-butylphosphino-2′,4′,6′-triisopropyl-1,1′-biphenyl)-2-(2′-amino-1,1′-biphenyl)] palladium(II) methanesulfonate (0.170 g, 0.214 mmol) in glove box. The mixture was stirred at 70° C. for 12 h, and cooled to ambient temperature. The reaction was repeated on the same scale. The crude mixtures from two reactions were combined, diluted with ethyl acetate (20 mL) and filtered. The filtrate was concentrated in vacuo. Purification of the residue by column chromatography, using 75% ethyl acetate containing 0.2% of trimethylamine in petroleum ether as eluent, afforded the title compound as a yellow solid (0.687 g, 52% yield): NH NMR (400 MHz, CDCl₃) δ 9.04 (s, 2H), 8.01 (d, J=6.0, 1H), 7.87 (d, J=9.2 Hz, 1H), 7.23 (dd, J=2.4 9.2 Hz, 1H), 7.10 (d, J=6.0, 1H), 7.03 (d, J=2.41z, 1H), 6.95 (d, 1H), 3.95 (d, J=7.0 Hz, 2H), 2.72 (s, 3H), 1.42-29 (i, 1H), 0.78-0.62 (m, 2H), 0.49-0.35 (i, 2H); MS (ES+) m/z 307.0 (M+1).

### Examples 25-27

In a similar manner as described in EXAMPLE 24, utilizing the appropriately substituted starting materials and intermediates, the following compounds were prepared:

| | | MS | |
|---|---|---|---|
| | | (ES+) | |
| Example No | Name | m/z | NMR |
| 25 | N-(2-methylpyrimidin- | 337.2 | ¹H NMR (300 MHz, DMSO-d₆) δ 9.29 (s, |
| | 5-yl)-6-((tetrahydro- | (M + 1) | 1H), 9.16 (s, 2H), 8.42 (d, J = 8.9 Hz, 1H), |
| | furan-3-yl)methoxy)iso- | | 7.94 (d, J = 5.8 Hz, 1H), 7.32-7.25 (m, 2H), |
| | quinolin-1-amine | | 7.15 (d, J = 5.7 Hz, 1H), 4.16-4.00 (m, 2H), |
| | | | 3.88-3.75 (m, 2H), 3.73-3.65 (m, 1H), 3.62- |
| | | | 3.54 (m, 1H), 2.80-2.66 (m, 1H), 2.57 (s, |
| | | | 3H), 2.12-2.00 (m, 1H), 1.78-1.64 (m, 1H). |
| 26 | 6-(2,2-difluoroethoxy)- | 317.0 | ¹H NMR (300 MHz, DMSO-d₆) δ 9.34 (s, |
| | N-(2-methylpyrimidin- | (M + 1) | 1H), 9.15 (s, 2H), 8.46 (d, J = 9.1 Hz, 1H), |
| | 5-yl)isoquinolin-1- | | 7.97 (d, J = 5.8 Hz, 1H), 7.42-7.32 (m, 2H), |
| | amine | | 7.17 (d, J = 5.7 Hz, 1H), 6.48 (tt, J = 54.4, |
| | | | 3.6 Hz, 1H), 4.50 (td, J = 14.6, 3.6 Hz, 2H), |
| | | | 2.58 (s, 3H). |
| 27 | 6-((5,5-dimethyltetra- | 365.2 | ¹H NMR (500 MHz, DMSO-d₆) δ 9.29 (s, |
| | hydrofuran-2-yl)methoxy)- | (M +1) | 1H), 9.16 (s, 2H), 8.45-8.38 (m, 1H), 7.93 |
| | N-(2-methylpyrimidin-5- | | (d, J = 5.8 Hz, 1H), 7.32-7.25 (m, 2H), 7.14 |
| | yl)isoquinolin-1-amine | | (d, J = 5.8 Hz, 1H), 4.34-4.26 (m, 1H), |
| | | | 4.12-4.07 (m, 1H), 4.06-4.00 (m, 1H), 2.57 |
| | | | (s, 3H), 2.16-2.07 (m, 1H), 1.89-1.69 (m, |
| | | | 3H), 1.24-1.16 (m, 6H). |

### Example 28

### Synthesis of 6-(cyclopropylmethoxy)-N-(6-methylpyridin-3-yl)isoquinolin-1-amine

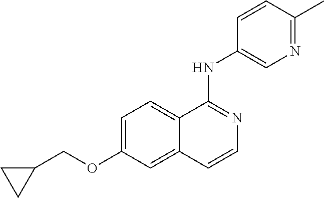 [SMILES: Cc1ccc(Nc2nccc3cc(OCC4CC4)ccc23)cn1]

Following the procedure as described for EXAMPLE 24 and making variations as required to replace 2-methylpyrimidin-5-amine with 6-methylpyridin-3-amine, the title compound was obtained as a yellow solid (0.275 g, 35% yield): ¹H NMR (400 MHz, CDCl₃) δ 8.58 (d, J=2.8 Hz, 1H), 8.19 (dd, J=2.8, 8.4 Hz, 1H), 8.01 (d, J=5.6 Hz, 1H), 7.86 (d, J=9.2 Hz, 1H), 7.22 (dd, J=2.4, 9.2 Hz, 1H), 7.16 (d, J=8.4 Hz, 1H), 7.05 (d, J=6.0 Hz, 1H), 7.02 (d, J=2.8 Hz, 1H), 6.96 (s, 1H), 3.95 (d, J=7.0 Hz, 2H), 2.54 (s, 3H), 1.41-1.29 (m, 1H), 0.77-0.65 (m, 2H), 0.48-0.35 (m, 2H); MS (ES+) m/z 306.1 (M+1).

### Example 29

### Synthesis of N-(6-chloropyridin-3-yl)-6-(1-cyclopropylethoxy)isoquinolin-1-amine

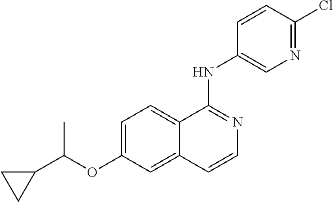 [SMILES: CC(Oc1ccc2c(Nc3ccc(Cl)nc3)nccc2c1)C1CC1]

### Step 1. Preparation of 1-chloro-6-(1-cyclopropylethoxy)isoquinoline

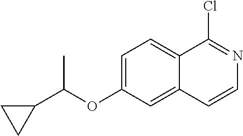 [SMILES: CC(Oc1ccc2c(Cl)nccc2c1)C1CC1]

Following the procedure as described for EXAMPLE 1, Step 1 and making non-critical variations as required to replace 2-propanol with 1-cyclopropylethanol, the title compound was obtained as a pale yellow solid (0.100 g, 35% yield): ¹H NMR (400 MHz, CDCl₃) δ 8.33-8.18 (m, 2H), 7.59-7.51 (m, 1H), 7.44-7.33 (m, 1H), 7.11 (d, J=0.9 Hz, 1H), 4.27-3.94 (m, 1H), 1.52-1.46 (m, 3H), 0.92-0.79 (m, 1H), 0.70-0.55 (m, 2H), 0.50-0.27 (m, 2H); MS (ES+) m/z 248.1, 250.1 (M+1).

### Step 2. Preparation of N-(6-chloropyridin-3-yl)-6-(1-cyclopropylethoxy)isoquinolin-1-amine

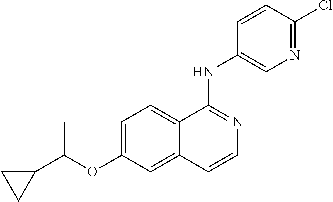 [SMILES: CC(Oc1ccc2c(Nc3ccc(Cl)nc3)nccc2c1)C1CC1]

To a mixture of 1-chloro-6-(1-cyclopropylethoxy)isoquinoline (0.0500 g, 0.202 mmol) and 6-chloropyridin-3-amine (0.0260 g, 0.202 mmol) in tert-amyl alcohol (2 mL) was added [(2-di-tert-butylphosphino-2′,4′,6′-triisopropyl-1,1′-biphenyl)-2-(2′-amino-1,1′-biphenyl)] palladium(II) methanesulfonate (0.0160 g, 0.0202 mmol) and cesium carbonate (0.329 g, 1.01 mmol) in glove box. The mixture was stirred at 25° C. for 12 h, and concentrated in vacuo. The residue was purified by preparative reverse-phase HPLC, using acetonitrile in water containing 0.225% of formic acid as eluent, to afford the title compound as a colorless solid (0.0138 g, 19% yield): ¹H NMR (400 MHz, CD₃OD) δ 8.71 (d, J=2.6 Hz, 1H), 8.28-8.21 (m, 2H), 7.88 (d, J=5.8 Hz, 1H), 7.39 (dd, J=8.7, 0.4 Hz, 1H), 7.24-7.11 (m, 3H), 4.20-4.12 (m, 1H), 1.45-1.41 (m, 3H), 1.21-1.14 (m, 1H), 0.61-0.53 (m, 2H), 0.46-0.35 (m, 2H), exchangeable proton not observed; MS (ES+) m/z 340.2, 342.2 (M+1).

### Example 30

### Synthesis of N-(6-chloropyridin-3-yl)furo[3,2-c]pyridin-4-amine formate

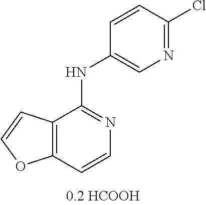 [SMILES: Clc1ccc(Nc2nccc3occc23)cn1]

To a mixture of 2-chloro-5-iodopyridine (0.100 g, 0.418 mmol), furo[3,2-c]pyridin-4-amine (0.0784 g, 0.585 mmol) and sodium tert-butoxide (2 M solution in tetrahydrofuran, 0.60 mL, 1.2 mmol) in 1,4-dioxane (2.5 mL) was added methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (0.0379 g, 00418 mmol) in glove box. The mixture was stirred at 90° C. for 12 h, cooled down to ambient temperature, and concentrated in vacuo. The residue was purified by preparative reverse-phase HPLC, eluting with acetonitrile in a 10 mM aqueous ammonium bicarbonate solution, to afford the title compound as a colorless solid (0.0376 g, 36% yield): ¹H NMR (400 MHz, CD₃OD) δ 8.80 (d, J=2.8 Hz, 1H), 8.50 (s, 0.2H), 8.38 (dd, J=2.8, 8.8 Hz, 1H), 8.03 (d, J=6.0 Hz, 1H), 7.79 (d, J=2.4 Hz, 1H), 7.38 (d, J=8.8 Hz, 1H), 7.17 (dd, J=1.2, 2.4 Hz, 1H), 7.09 (dd, J=1.0, 6.0 Hz, 1H), exchangeable protons not observed; MS (ES+) m/z 245.9, 247.9 (M+1).

### Example 31

### Synthesis of N-(6-chloropyridin-3-yl)-1,7-naphthyridin-8-amine formate

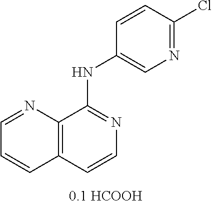 [SMILES: Clc1ccc(Nc2nccc3cccnc23)cn1]

To a mixture of 6-chloropyridin-3-amine (0.0703 g, 0.547 mmol), 8-chloro-1,7-naphthyridine (0.090 g, 0.547 mmol) and cesium carbonate (0.534 g, 1.64 mmol) in tert-amyl alcohol (1.5 mL) was added [(2-di-tert-butylphosphino-2′,4′,6′-triisopropyl-1,1′-biphenyl)-2-(2′-amino-1,1′-biphenyl)] palladium(II) methanesulfonate (0.0434 g, 0.0547 mmol) in glove box. The mixture was stirred at 90° C. for 12 h, cooled to ambient temperature, and concentrated in vacuo to provide a crude product. The reaction was repeated on a 0.122 mmol scale. The crude products were combined, and purified by preparative reverse-phase HPLC, using acetonitrile in water containing 0.225% of formic acid as eluent, to afford the title compound as a yellow solid (0.0460 g, 23% yield): ¹H NMR (400 MHz, DMSO-d₆) δ 10.05 (s, 1H), 9.17-9.08 (m, 1H), 8.97 (dd, J=1.6, 4.4 Hz, 1H), 8.72-8.62 (m, 1H), 8.44 (s, 01H), 8.34 (dd, J=1.6, 8.4 Hz, 1H), 8.13 (d, J=5.8 Hz, 1H), 7.81 (dd, J=4.4, 8.4 Hz, 1H), 7.47 (d, J=8.8 Hz, 1H), 7.28 (d, J=5.8 Hz, 1H); MS (ES+) m/z 257.0, 259.0 (M+1)

### Example 32

### Synthesis of N-(2-chloropyrimidin-5-yl)-1,7-naphthyridin-8-amine

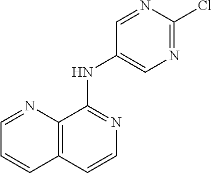 [SMILES: Clc1ncc(Nc2nccc3cccnc23)cn1]

To a mixture of 8-chloro-1,7-naphthyridine (0.100 g, 0.608 mmol), 2-chloropyrimidin-5-amine (0.0787 g, 0.608 mmol), and cesium carbonate (0.594 g, 1.82 mmol) in tert-amyl alcohol (2 mL), was added [(2-di-tert-butylphosphino-2′,4′,6′-triisopropyl-1,1′-biphenyl)-2-(2′-amino-1,1′-biphenyl)] palladium(II) methanesulfonate (0.00483 g, 0.00608 mmol) in glove box. The mixture was stirred at 80° C. for 12 h, and cooled to ambient temperature. Following the same procedure as described above, two more reactions were carried out on the same scale in parallel. The crude mixtures from three parallel reactions were combined. The resulting mixture was diluted with ethyl acetate (20 mL), and thiourea resin (0.300 g) was added. The mixture was stirred at ambient temperature for 4 h, filtered, and concentrated in vacuo. The residue was purified by preparative HPLC, eluting with ethanol containing 0.1% of ammonium hydroxide in heptane, followed by preparative reverse-phase HPLC, eluting with acetonitrile in a 10 mM of aqueous ammonium bicarbonate, to afford the title compound as a yellow solid (0.107 g, 23% yield): ¹H NMR (400 MHz, DMSO-d₆) δ 10.30 (s, 1H), 9.53 (s, 2H), 9.00 (dd, J=1.6, 4.4 Hz, 1H), 8.38 (dd, J=1.6, 8.4 Hz, 1H), 8.17 (d, J=5.6 Hz, 1H), 7.84 (dd, J=4.4, 8.4 Hz, 1H), 7.34 (d, J=6.0 Hz, 1H); MS (ES+) m/z 258.0, 260.0 (M+1).

### Example 33

### Synthesis of N-(6-chloropyridin-3-yl)isoquinolin-1-amine

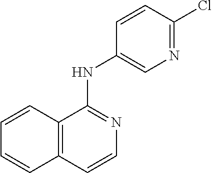 [SMILES: Clc1ccc(Nc2nccc3ccccc23)cn1]

Following the procedure as described for EXAMPLE 31 and making variations as required to replace 8-chloro-1,7-naphthyridine with 1-bromoisoquinoline, the title compound was obtained as a colorless solid (0.342 g, 34% yield): ¹H NMR (400 MHz, CDCl₃) δ 8.55 (d, J=2.8 Hz, 1H), 8.42 (dd, J=2.8, 8.8 Hz, 1H), 8.10 (d, J=5.8 Hz, 1H), 7.96 (d, J=8.4 Hz, 1H), 7.81 (d, J=8.2 Hz, 1H), 7.70 (dt, J=1.0, 7.6 Hz, 1H), 7.65-7.57 (m, 1H), 7.34 (d, J=8.8 Hz, 1H), 7.23 (d, J=5.8 Hz, 1H), 7.18 (s, 1H); MS (ES+) m/z 256.0, 258.0 (M+1).

### Example 34

### Synthesis of ((1-((6-chloropyridin-3-yl)amino)isoquinolin-6-yl)imino)dimethyl-λ⁶-sulfanone

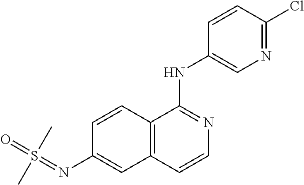 [SMILES: CS(C)(=O)=Nc1ccc2c(Nc3ccc(Cl)nc3)nccc2c1]

### Step 1. Preparation of ((1-chloroisoquinolin-6-yl)imino)dimethyl-λ⁶-sulfanone

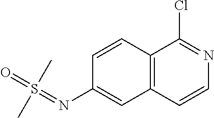 [SMILES: CS(C)(=O)=Nc1ccc2c(Cl)nccc2c1]

To a solution of 6-bromo-1-chloro-isoquinoline (0.100 g, 0.412 mmol), iminodimethyl-λ⁶-sulfanone (0.0420 g, 0.454 mmol), tris(dibenzylideneacetone)dipalladium(0) (0.0380 g, 0.0412 mmol), and 4,5-bis(diphenylphosphino)-9,9-dimethylxanthene (0.0480 mg, 0.0830 mmol) in 1,4-dioxane (4 mL) was added sodium tert-butoxide (0.0790 g, 0.825 mmol) and the mixture was stirred at 100° C. for 90 minutes. The reaction mixture was cooled to ambient temperature, diluted with aqueous saturated sodium bicarbonate (20 mL), and extracted with ethyl acetate (3×20 mL). The combined organic layers were washed with brine (30 mL), dried over anhydrous sodium sulfate, filtered, and concentrated in vacuo. The residue was dissolved in ethyl acetate (5 mL) and passed through a bed of silica. The bed of silica was washed with ethyl acetate (120 mL) and the combined filtrate was concentrated in vacuo. Purification of the residue by column chromatography, eluting with a gradient of 0 to 20% of ethyl acetate in hexanes, afforded the title compound as a colorless solid (0.019 g, 18% yield): ¹H NMR (400 MHz; CDCl₃) δ 8.18 (d, J=9.0 Hz, 1H), 8.14 (d, J=5.7 Hz, 1H), 7.43 (t, J=4.3 Hz, 2H), 7.37-7.34 (m, 1H), 3.24 (s, 6H); MS (ES+) m/z 255.1, 257.1 (M+1).

### Step 2. Preparation of ((1-((6-chloropyridin-3-yl)amino)isoquinolin-6-yl)imino)dimethyl-λ⁶-sulfanone

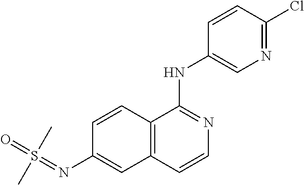 [SMILES: CS(C)(=O)=Nc1ccc2c(Nc3ccc(Cl)nc3)nccc2c1]

To a solution of ((1-chloroisoquinolin-6-yl)imino)dimethyl-λ⁶-sulfanone (0.0200 g, 0.0746 mmol), 6-chloropyridin-3-amine (0.0110 g, 0.082 mmol), 2-dicyclohexylphosphino-2′,4′,6′-triisopropylbiphenyl (0.00400 mg, 0.00746 mmol), and potassium phosphate tribasic (0.048 g, 0.224 mmol) in 1,4-dioxane (2.00 mL) was added tris(dibenzylideneacetone)dipalladium(0) (0.003 g, 0.00373 mmol) and the mixture was stirred at 100° C. for 4 h. After cooling to ambient temperature, the mixture was passed through a pad of diatomaceous earth. The pad was washed with ethyl acetate (50 mL) and the combined filtrate was concentrated in vacuo. Purification of the residue by column chromatography, eluting with a gradient of 0 to 15% of methanol in dichloromethane, followed by preparative reverse-phase HPLC, using acetonitrile in a 10 mM of aqueous ammonium bicarbonate as eluent, afforded the title compound as a colorless solid (0.011 g, 43% yield): ¹H NMR (400 MHz; DMSO-d₆) δ 9.24 (s, 1H), 8.83 (d, J=2.9 Hz, 1H), 8.38 (dd, J=8.8, 2.9 Hz, 1H), 8.29 (d, J=9.1 Hz, 1H), 7.85 (d, J=5.8 Hz, 1H), 7.39 (d, J=8.7 Hz, 1H), 7.23 (d, J=2.3 Hz, 1H), 7.15 (dd, J=9.0, 2.3 Hz, 1H), 7.05 (d, J=5.6 Hz, 1H), 3.30 (s, 6H); MS (ES+) m/z 347.1, 349.1 (M+1).

### Example 35

### Synthesis of N-(6-chloropyridin-3-yl)-4-methyl-1H-pyrrolo[2,3-c]pyridin-7-amine

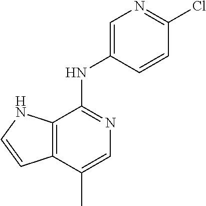 [SMILES: Cc1cnc(Nc2ccc(Cl)nc2)c2[nH]ccc12]

To a mixture of 6-chloropyridin-3-amine (0.239 g, 1.76 mmol) and 7-chloro-4-methyl-1H-pyrrolo[2,3-c]pyridine (0.04 g, 0.235 mmol) in ethanol (2.2 mL) was added a 4 M solution of hydrochloric acid in 1,4-dioxane (0.49 mL, 1.97 mmol), and the mixture was stirred at 50° C. for 3 h, at 75° C. for 45 minutes, and at 85° C. for 66 h. After cooling to ambient temperature, the mixture was diluted with saturated aqueous sodium bicarbonate (20 mL), and extracted with ethyl acetate (3×20 mL). The combined organic phases were washed with brine (20 mL), dried over anhydrous sodium sulfate, filtered and concentrated in vacuo. Purification of the residue by column chromatography, eluting with a gradient of 0 to 50% of methanol in dichloromethane, followed by preparative reverse-phase HPLC, using acetonitrile in a 10 mM aqueous ammonium bicarbonate as eluent, afforded the title compound as a colorless solid (0.016 g, 26% yield): ¹H NMR (400 MHz; DMSO-d₆) δ 11.22 (s, 1H), 8.98 (s, 1H), 8.74 (dd, J=2.9, 0.6 Hz, 1H), 8.43 (dd, J=8.8, 2.9 Hz, 1H), 7.60-7.51 (m, 2H), 7.41 (d, J=8.7 Hz, 1H), 6.49 (dd, J=3.0, 2.0 Hz, 1H), 2.36 (d, J=1.0 Hz, 3H); MS (ES+) m/z 259.0, 261.0 (M+1).

### Example 36

### Synthesis of N-(6-chloro-5-methoxypyridin-3-yl)-6-((3-methyloxetan-3-yl)methoxy)isoquinolin-1-amine

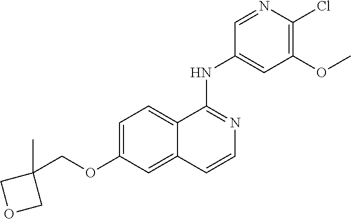 [SMILES: COc1cc(Nc2nccc3cc(OCC4(C)COC4)ccc23)cnc1Cl]

### Step 1. Preparation of (3-methyloxetan-3-yl)methyl 4-methylbenzenesulfonate

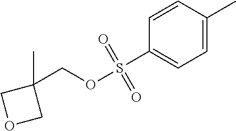 [SMILES: Cc1ccc(S(=O)(=O)OCC2(C)COC2)cc1]

To a solution of 3-methyl-3-oxetanemethanol (1.00 g, 9.79 mmol) in dichloromethane (10 mL) was added p-toluenesulfonyl chloride (2.24 g, 11.7 mmol) and triethylamine (2.70 mL, 19.6 mmol) and the reaction mixture was stirred at ambient temperature for 16 h. The reaction mixture was diluted with dichloromethane (30 mL), washed with water (30 mL), and saturated sodium chloride (20 mL). The organic phase was dried over anhydrous sodium sulfate, filtered, and the filtrate was concentrated in vacuo. The residue was purified by silica gel column chromatography, eluting with a gradient of 0 to 50% of ethyl acetate in heptane, to provide the title compound as a colorless solid (0.650 g, 26% yield): MS (ES+) m/z 257.2 (M+1).

### Step 2. Preparation of 1-chloro-6-((3-methyloxetan-3-yl)methoxy)isoquinoline

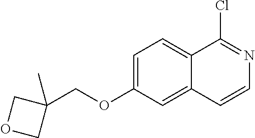 [SMILES: CC1(COc2ccc3c(Cl)nccc3c2)COC1]

To a solution of (3-methyloxetan-3-yl)methyl 4-methylbenzenesulfonate (0.750 g, 2.93 mmol) in N,N-dimethylformamide (6 mL), was added potassium carbonate (0.809 mg, 5.85 mmol) and 1-chloroisoquinolin-6-ol (0.526 mg, 2.93 mmol). The reaction mixture was heated to 80° C. for 48 h. After cooling to ambient temperature, the reaction mixture was diluted with ethyl acetate (20 mL), washed with water (20 mL) and saturated sodium chloride (20 mL). The organic phase was dried over anhydrous sodium sulfate, filtered, and the filtrate concentrated in vacuo. The residue was purified by silica gel column chromatography, eluting with a gradient of 0 to 40% of ethyl acetate in heptane, to provide the title compound as a colorless oil (0.700 g, 74% yield): MS (ES+) m/z 264.0 (M+1).

### Step 3. Preparation of N-(6-chloro-5-methoxypyridin-3-yl)-6-((3-methyloxetan-3-yl)methoxy)isoquinolin-1-amine

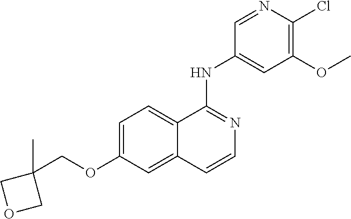 [SMILES: COc1cc(Nc2nccc3cc(OCC4(C)COC4)ccc23)cnc1Cl]

A mixture of 6-chloro-5-methoxypyridin-3-amine (0.045 g, 0.284 mmol), 1-chloro-6-((3-methyloxetan-3-yl)methoxy)isoquinoline (0.150 g, 0.569 mmol), [(2-di-tert-butylphosphino-2′,4′,6′-triisopropyl-1,1′-biphenyl)-2-(2′-amino-1,1′-biphenyl)]palladium(II) methanesulfonate (0.045 g, 0.057 mmol),and cesium carbonate (0.371 g, 1.14 mmol) in 2-methylbutan-2-ol (1 mL) was stirred at 70° C. for 12 h. The reaction was repeated once more, and the reaction mixtures were combined. After cooling to ambient temperature, the combined reaction mixtures were poured into water (30 mL) and extracted with ethyl acetate (3×30 mL). The combined organic layers were washed with brine (30 mL), dried over sodium sulfate, and filtered. Concentration of the filtrate in vacuo and purification of the residue was purified by reverse-phase preparative HPLC, eluting with a gradient of 37% to 67% of acetonitrile in water containing 10 mM ammonium bicarbonate, afforded the title compound as a yellowish solid (0.036 g, 16% yield): ¹H NMR (400 MHz, DMSO-d₆) δ 9.36 (s, 1H), 8.62 (d, J=1.8 Hz, 1H), 8.47 (d, J=9.0 Hz, 1H), 8.24 (d, J=1.8 Hz, 1H), 8.00 (d, J=5.8 Hz, 1H), 7.37-7.31 (m, 2H), 7.19 (d, J=5.8 Hz, 1H), 4.55 (d, J=5.8 Hz, 2H), 4.35 (d, J=5.8 Hz, 2H), 4.23 (s, 2H), 3.90 (s, 3H), 1.42 (s, 3H); MS (ES+) m/z 473.2 (M+1), 475.2 (M+1).

### Examples 37-57

In a similar manner as described in EXAMPLE 36, utilizing the appropriately substituted starting materials and intermediates, the following compounds were prepared:

| | | MS | |
|---|---|---|---|
| | | (ES+) | |
| Example No | Name | m/z | NMR |
| 37 | 6-(2-methoxyethoxy)- | 311.2 | ¹H NMR (400 MHz, DMSO-d₆) δ |
| | N-(2-methylpyrimidin-5- | (M + 1). | 11.02 (m, 1H), 8.94 (s, 2H), 8.60 (d, |
| | yl)isoquinolin-1-amine | | J = 8.8 Hz, 1H), 7.61 (d, J = 6.8 Hz, |
| | | | 1H), 7.58-7.52 (m, 2H), 7.34 (d, J = |
| | | | 7.2 Hz, 1H), 4.40-4.30 (m, 2H), 3.79- |
| | | | 3.71 (m, 2H), 3.35-3.35 (m, 1H), 3.34 |
| | | | (s, 2H), 2.73 (s, 3H). |
| 38 | 6-(2-cyclopropoxyethoxy)- | 337.2 | ¹H NMR (400 MHz, DMSO-d₆ and |
| | N-(2-methylpyrimidin-5- | (M + 1). | D₂O) δ 9.16-9.11 (m, 2H), 8.39 (d, J = |
| | yl)isoquinolin-1-amine | | 10.0 Hz, 1H), 7.93 (d, J = 6.0 Hz, 1H), |
| | | | 7.30-7.23 (m, 2H), 7.16 (d, J = 6.0 |
| | | | Hz, 1H), 4.26-4.21 (m, 2H), 3.85- |
| | | | 3.80 (m, 2H), 3.40-3.38 (m, 1H), 2.57 |
| | | | (s, 3H), 0.54-0.48 (m, 2H), 0.46 (t, |
| | | | J = 4.4 Hz, 2H), NH not observed. |
| 39 | N-(6-chloropyridin-3- | 356.1 | ¹H NMR (400 MHz, DMSO-d₆ and |
| | yl)-6-(2-cyclopropoxy- | (M + 1), | D₂O) δ 8.65 (d, J = 2.8 Hz, 1H), 8.56 |
| | ethoxy)isoquinolin-1-amine | 358.1 | (d, J = 8.8 Hz, 1H), 8.09 (dd, J = 2.4, |
| | | (M + 1). | 8.4 Hz, 1H), 7.68 (d, J = 8.4 Hz, 1H), |
| | | | 7.61 (d, J = 6.8 Hz, 1H), 7.50-7.43 |
| | | | (m, 2H), 7.31 (d, J = 6.8 Hz, 1H), |
| | | | 4.33-4.27 (m, 2H), 3.87-3.81 (m, |
| | | | 2H), 3.38 (tt, J = 3.2, 5.6 Hz, 1H), |
| | | | 0.51-0.47 (m, 2H), 0.46 (dd, J = 2.4, |
| | | | 3.6 Hz, 2H), NH not observed. |
| 40 | 3-(((1-((2- | 378.2 | ¹H NMR (400 MHz, DMSO-d₆) δ 9.58 |
| | chloropyrimidin-5- | (M + 1), | (s, 1H), 9.32-9.25 (m, 2H), 8.42 (d, |
| | yl)amino)isoquinolin- | 380.2 | J = 9.2 Hz, 1H), 7.99 (d, J = 5.6 Hz, |
| | 6-yl)oxy)meth- | (M + 1). | 1H), 7.34-7.28 (m, 2H), 7.22 (d, J = |
| | yl)bicyclo[1.1.1]pentane- | | 5.6 Hz, 1H), 4.19 (s, 2H), 2.32 (s, 6H). |
| | 1-carbonitrile | | |
| 41 | N-(2-chloropyrimidin-5- | 385.1 | ¹H NMR (400 MHz, DMSO-d₆) δ 9.59 |
| | yl)-6-((3-isopropyloxetan- | (M + 1), | (s, 1H), 9.30 (s, 2H), 8.44 (d, J = 9.2 |
| | 3-yl)methoxy)isoquinolin- | 387.1 | Hz, 1H), 8.01 (d, J = 5.6 Hz, 1H), 7.43 |
| | 1-amine | (M + 1). | (d, J = 2.4 Hz, 1H), 7.36 (dd, J = 9.2, |
| | | | 2.4 Hz, 1H), 7.25 (d, J = 6.0 Hz, 1H), |
| | | | 4.51 (d, J = 6.0 Hz, 2H), 4.44 (d, J = |
| | | | 6.0 Hz, 2H), 4.28 (s, 2H), 2.21 (td, J = |
| | | | 13.6, 6.8 Hz, 1H), 0.99 (d, J = 6.8 Hz, 6H). |
| 42 | 2-chloro-5-((6-((3- | 372.1 | ¹H NMR (400 MHz, DMSO-d₆) δ |
| | methyloxetan-3- | (M + 1), | 11.03-10.29 (m, 1H), 9.26 (s, 1H), |
| | yl)methoxy)isoquinolin- | 374.1 | 8.47 (d, J = 9.4 Hz, 1H), 8.32 (d, J = |
| | 1-yl)amino)pyridin-3-ol | (M + 1). | 2.2 Hz, 1H), 8.19 (d, J = 2.0 Hz, 1H), |
| | | | 7.97 (d, J = 5.8 Hz, 1H), 7.34 (d, J = |
| | | | 2.4 Hz, 1H), 7.30 (dd, J = 2.4, 9.2 Hz, |
| | | | 1H), 7.17 (d, J = 5.8 Hz, 1H), 4.55 (d, |
| | | | J = 5.8 Hz, 2H), 4.35 (d, J = 5.8 Hz, |
| | | | 2H), 4.22 (s, 2H), 1.42 (s, 3H). |
| 43 | N-(6-(difluoromethyl)pyridin- | 360.1 | ¹H NMR (400 MHz, DMSO-d₆) δ 9.52 |
| | 3-yl)-6-((1- | (M + 1). | (s, 1H), 9.10 (d, J = 2.4 Hz, 1H), 8.56 |
| | fluorocyclopropyl)methoxy)isoquinolin- | | (dd, J = 2.4, 8.8 Hz, 1H), 8.49 (d, J = |
| | 1-amine | | 9.2 Hz, 1H), 8.00 (d, J = 5.6 Hz, 1H), |
| | | | 7.65 (d, J = 8.8 Hz, 1H), 7.43-7.35 |
| | | | (m, 1H), 7.33 (d, J = 2.4 Hz, 1H), 7.21 |
| | | | (d, J = 5.6 Hz, 1H), 6.89 (t, 55.6 Hz, |
| | | | 1H), 4.50-4.44 (m, 2H), 1.24-1.12 (m, |
| | | | 2H), 0.98-0.87 (m, 2H). |
| 44 | N-(2-ethylpyrimidin-5- | 339.0 | ¹H NMR (400 MHz, DMSO-d₆) δ 9.32 |
| | yl)-6-((1- | (M + 1). | (s, 1H), 9.17 (s, 2H), 8.44 (d, J = 9.2 |
| | fluorocyclopropyl)methoxy)isoquinolin- | | Hz, 1H), 7.94 (d, J = 5.6 Hz, 1H), 7.36 |
| | 1- | | (dd, J = 9.2 2.4 Hz, 1H), 7.31 (d, J = |
| | amine | | 2.4 Hz, 1H), 7.15 (d, J = 5.6 Hz, 1H), |
| | | | 4.49 (s, 1H), 4.44 (s, 1H), 2.86 (q, |
| | | | J = 7.6 Hz, 2H), 1.28 (t, J = 7.6 Hz, 3H), |
| | | | 1.22-1.13 (m, 2H), 0.95-0.89 (m, 2H). |
| 45 | N-(5-chloro-6- | 358.2 | ¹H NMR (400 MHz, DMSO-d₆) δ |
| | methylpyridin-3-yl)-6- | (M + 1). | 8.48-8.46 (m, 2H), 8.05-8.02 (m, |
| | ((1-fluorocyclopropyl)meth- | | 1H), 7.88 (d, J = 9.2 Hz, 1H), 7.31- |
| | oxy)isoquinolin-1-amine | | 7.27 (m, 1H), 7.12-7.09 (m, 1H), 7.08 |
| | | | (s, 1H), 4.40-4.35 (m, 2H), 2.61 (s, |
| | | | 3H), 1.32-1.24 (m, 2H), 0.93-0.87 |
| | | | (m, 2H), NH not observed. |
| 46 | 6-((1-fluorocyclo- | 341.2 | ¹H NMR (400 MHz, DMSO-d₆) δ 9.64 |
| | propyl)methoxy)-N-(2- | (M + 1). | (s, 1H), 8.95 (s, 2H), 8.48 (d, J = 9.2 |
| | methoxypyrimidin-5- | | Hz, 1H), 7.82 (d, J = 5.6 Hz, 1H), |
| | yl)isoquinolin-1-amine | | 7.41-7.35 (m, 2H), 7.13 (d, J = 6.0 |
| | | | Hz, 1H), 4.45-4.55 (m, 2H), 3.93 (s, |
| | | | 3H), 1.22-1.14 (m, 2H), 0.98-0.90 |
| | | | (m, 2H). |
| 47 | 6-((1-fluorocyclo- | 378.1 | ¹H NMR (400 MHz, DMSO-d₆) δ 9.68 |
| | propyl)methoxy)-N-(6- | (M + 1). | (s, 1H), 9.17 (d, J = 2.0 Hz, 1H), |
| | (trifluoromethyl)pyridin- | | 8.72-8.59 (m, 1H), 8.50 (d, J = 9.2 |
| | 3-yl)isoquinolin-1-amine | | Hz, 1H), 8.03 (d, J = 5.6 Hz, 1H), 7.83 |
| | | | (d, J = 8.8 Hz, 1H), 7.39 (dd, J = 2.0, |
| | | | 9.2 Hz, 1H), 7.35 (d, J = 2.0 Hz, 1H), |
| | | | 7.25 (d, J = 5.6 Hz, 1H), 4.57-4.37 |
| | | | (m, 2H), 1.18 (d, J = 18.8 Hz, 2H), |
| | | | 0.93 (d, J = 7.2 Hz, 2H). |
| 48 | N-(5-chloropyridin-3- | 344.2 | ¹H NMR (400 MHz, CDCl₃) δ 8.81- |
| | yl)-6-((1-fluorocyclo- | (M + 1). | 8.41 (m, 2H), 8.38-8.15 (m, 1H), |
| | propyl)methoxy)isoquinolin- | | 8.13-8.03 (m, 1H), 7.96-7.83 (m, |
| | 1-amine | | 1H), 7.31 (dd, J = 2.4, 9.2 Hz, 1H), |
| | | | 7.27-7.18 (m, 1H), 7.17-7.12 (m, |
| | | | 1H), 7.12-7.06 (m, 1H), 4.43-4.38 |
| | | | (m, 1H), 4.38-4.32 (m, 1H), 1.33- |
| | | | 1.28 (m, 1H), 1.28-1.24 (m, 1H), |
| | | | 0.95-0.85 (m, 2H). |
| 49 | 6-((1- | 311.0 | ¹H NMR (400 MHz, DMSO-d₆) δ 9.42 |
| | fluorocyclopropyl)methoxy)- | (M + 1). | (s, 1H), 9.30 (s, 2H), 8.78 (s, 1H), |
| | N-(pyrimidin-5- | | 8.46 (d, J = 9.2 Hz, 1H), 7.99 (d, J = |
| | yl)isoquinolin-1-amine | | 5.6 Hz, 1H), 7.43-7.29 (m, 2H), 7.20 |
| | | | (d, J = 5.6 Hz, 1H), 4.52-4.39 (m, |
| | | | 2H), 1.28-1.11 (m, 2H), 0.99-0.84 |
| | | | (m, 2H). |
| 50 | 4-(((1-((6-chloropyridin- | 395.1 | ¹H NMR (400 MHz, DMSO-d₆) δ |
| | 3-yl)amino)isoquinolin- | (M + 1). | 11.04 (m, 1H), 8.79-8.70 (m, 2H), |
| | 6-yl)oxy)methyl)tetrahydro- | | 8.17 (d, J = 7.6 Hz, 1H), 7.73-7.66 |
| | 2H-pyran-4-carbonitrile | | (m, 2H), 7.58-7.51 (m, 2H), 7.31 (d, |
| | | | J = 6.8 Hz, 1H), 4.41-4.37 (m, 2H), |
| | | | 3.98 (dd, J = 2.4, 12.0 Hz, 2H), 3.56 |
| | | | (s, 2H), 2.01 (d, J = 13.2 Hz, 2H), |
| | | | 1.86-1.77 (m, 2H). |
| 51 | N-(6-chloropyridin-3- | 364.1 | ¹H NMR (400 MHz, DMSO-d₆) δ 9.41 |
| | yl)-6-(pyrimidin-4- | (M + 1), | (s, 1H), 9.23 (d, J = 1.3 Hz, 1H), 8.91- |
| | ylmethoxy)isoquinolin- | 366.0 | 8.87 (m, 2H), 8.50 (d, J = 9.1 Hz, 1H), |
| | 1-amine | (M + 1). | 8.43 (dd, J = 8.7, 2.8 Hz, 1H), 7.98 (d, |
| | | | J = 5.8 Hz, 1H), 7.71-7.70 (m, 1H), |
| | | | 7.47-7.42 (m, 2H), 7.41 (d, J = 2.6 |
| | | | Hz, 1H), 7.19 (d, J = 5.8 Hz, 1H), 5.41 |
| | | | (s, 2H). |
| 52 | N-(6-chloropyridin-3- | 360.1 | ¹H NMR (400 MHz, CD₃OD) δ 8.75 (d, |
| | yl)-6-((3-fluorooxetan- | (M + 1), | J = 2.8 Hz, 1H), 8.33-8.27 (m, 2H), |
| | 3-yl)methoxy)isoquinolin- | 362.1 | 7.95 (d, J = 6.0 Hz, 1H), 7.42 (d, J = |
| | 1-amine | (M + 1). | 9.2 Hz, 1H), 7.32-7.28 (m, 2H), 7.21 |
| | | | (d, J = 5.9 Hz, 1H), 4.88-4.79 (m, 2H), |
| | | | 4.60 (s, 4H), NH not observed; ¹⁹F |
| | | | NMR ( 376 MHz 376 MHz, CD₃OD) δ −157.4 (s). |
| 53 | 5-(((1-((6-chloropyridin- | 383.2, | ¹H NMR (400 MHz, CD₃OD) δ 8.74 (d, |
| | 3-yl)amino)isoquinolin- | (M + 1), | J = 2.6 Hz, 1H), 8.32-8.25 (m, 2H), |
| | 6-yl)oxy)methyl)-1- | 385.2 | 7.94 (d, J = 5.8 Hz, 1H), 7.42 (d, J = |
| | methylpyrrolidin-2-one | (M + 1). | 8.6 Hz, 1H), 7.31-7.24 (m, 2H), 7.20 |
| | | | (d, J = 6.1 Hz, 1H), 4.42 (dd, J = 10.3, |
| | | | 3.2 Hz, 1H), 4.24 (dd, J = 10.4, 4.2 |
| | | | Hz, 1H), 4.13-4.06 (m, 1H), 2.94 (s, |
| | | | 3H), 2.68-2.57 (m, 1H), 2.48-2.30 (m, |
| | | | 2H), 2.14-2.04 (m, 1H), NH not observed. |
| 54 | N-(6-chloro-5- | 396.2 | ¹H NMR (400 MHz, DMSO-d₆) δ 9.35 |
| | methoxypyridin-3-yl)-6- | (M + 1), | (s, 1H), 8.60 (d, J = 2.2 Hz, 1H), 8.43 |
| | ((1-methyl-1H-pyrazol- | 398.2 | (d, J = 9.3 Hz, 1H), 8.23 (d, J = 2.2 |
| | 4-yl)methoxy)isoquinolin- | (M + 1). | Hz, 1H), 8.00 (d, J = 5.8 Hz, 1H), 7.87 |
| | 1-amine | | (s, 1H), 7.57 (s, 1H), 7.40 (d, J = 2.5 |
| | | | Hz, 1H), 7.27 (dd, J = 9.2, 2.6 Hz, |
| | | | 1H), 7.20 (d, J = 5.8 Hz, 1H), 5.11 (s, |
| | | | 2H), 3.90 (s, 3H), 3.84 (s, 3H). |
| 55 | N-(5-methoxy-6- | 376.2 | ¹H NMR (400 MHz, DMSO-d₆) δ 9.11 |
| | methylpyridin-3-yl)-6- | (M + 1). | (s, 1H), 8.55 (d, J = 2.0 Hz, 1H), 8.43 |
| | ((1-methyl-1H-pyrazol- | | (d, J = 9.2 Hz, 1H), 8.15 (s, 0.3H), |
| | 4-yl)methoxy)isoquinolin- | | 8.00-7.91 (m, 2H), 7.86 (s, 1H), 7.57 |
| | 1-amine formate | | (s, 1H), 7.36 (d, J = 2.4 Hz, 1H), |
| | | | 7.27-7.19 (m, 1H), 7.12 (d, J = 6.0 |
| | | | Hz, 1H), 5.10 (s, 2H), 3.84 (s, 3H), |
| | | | 3.82 (s, 3H), 2.31 (s, 3H), COOH not |
| | | | observed. |
| 56 | N-(2-chloropyrimidin-5- | 361.2 | ¹H NMR (400 MHz, DMSO-d₆) δ 9.63 |
| | yl)-6-((3-fluorooxetan- | (M + 1), | (s, 1H), 9.30 (s, 2H), 8.46 (d, J = 8.9 |
| | 3-yl)methoxy)isoquinolin- | 363.2 | Hz, 1H), 8.02 (d, J = 5.8 Hz, 1H), |
| | 1-amine | (M + 1). | 7.39-7.36 (m, 2H), 7.24 (d, J = 5.8 Hz, |
| | | | 1H), 4.82-4.71 (m, 4H), 4.62 (d, J = |
| | | | 22.0 Hz, 2H). |
| 57 | 6-((3-fluorooxetan-3- | 341.2 | ¹H NMR (400 MHz, DMSO-d₆) δ 9.34 |
| | yl)methoxy)-N-(2- | (M + 1). | (s, 1H), 9.17 (s, 2H), 8.46 (d, J = 9.0 |
| | methylpyrimidin-5- | | Hz, 1H), 7.97 (d, J = 5.8 Hz, 1H), |
| | yl)isoquinolin-1-amine | | 7.37-7.33 (m, 2H), 7.17 (d, J = 5.8 Hz, |
| | | | 1H), 4.82-4.71 (m, 4H), 4.61 (d, J = |
| | | | 22.0 Hz, 2H), 2.58 (s, 3H). |

### Example 58

### Synthesis of 6-((3-fluoroazetidin-3-yl)methoxy)-N-(2-methylpyrimidin-5-yl)isoquinolin-1-amine

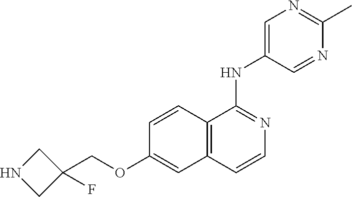 [SMILES: Cc1ncc(Nc2nccc3cc(OCC4(F)CNC4)ccc23)cn1]

### Step 1. Preparation of tert-butyl 3-fluoro-3-((tosyloxy)methyl)azetidine-1-carboxylate

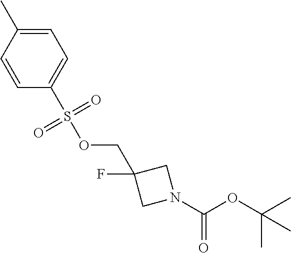 [SMILES: Cc1ccc(S(=O)(=O)OCC2(F)CN(C(=O)OC(C)(C)C)C2)cc1]

Following the procedure as described for EXAMPLE 36, Step 1, and making variations as required to replace 3-methyl-3-oxetanemethanol with tert-butyl 3-fluoro-3-(hydroxymethyl)azetidine-1-carboxylate, the title compound was obtained as a colorless solid (1.90 g, 69% yield): ¹H NMR (400 MHz, CDCl₃) 57.81 (d, J=8.4 Hz, 2H), 7.38 (d, J=8.4 Hz, 2H), 4.30-4.21 (m, 2H), 4.09-3.89 (m, 4H), 2.47 (s, 3H), 1.43 (s, 9H).

### Step 2. Preparation of tert-butyl 3-(((1-chloroisoquinolin-6-yl)oxy)methyl)-3-fluoroazetidine-1-carboxylate

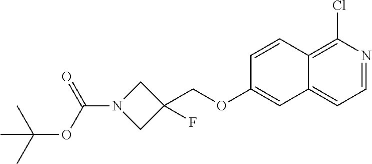 [SMILES: CC(C)(C)OC(=O)N1CC(F)(COc2ccc3c(Cl)nccc3c2)C1]

Following the procedure as described for EXAMPLE 36, Step 2, and making variations as required to replace (3-methyloxetan-3-yl)methyl 4-methylbenzenesulfonate with tert-butyl 3-fluoro-3-((tosyloxy)methyl)azetidine-1-carboxylate, the title compound was obtained as a colorless solid (1.00 g, 93% yield): ¹H NMR (400 MHz, CDCl₃) δ 8.28 (d, J=9.2 Hz, 1H), 8.23 (d, J=5.6 Hz, 1H), 7.50 (d, J=5.6 Hz, 1H), 7.35 (dd, J=2.4, 9.2 Hz, 1H), 7.13 (d, J=2.4 Hz, 1H), 4.39 (d, J=18.8 Hz, 2H), 4.28-4.14 (m, 4H), 1.48 (s, 9H).

### Step 3. Preparation of tert-butyl 3-fluoro-3-(((1-((2-methylpyrimidin-5-yl)amino)isoquinolin-6-yl)oxy)methyl)azetidine-1-carboxylate

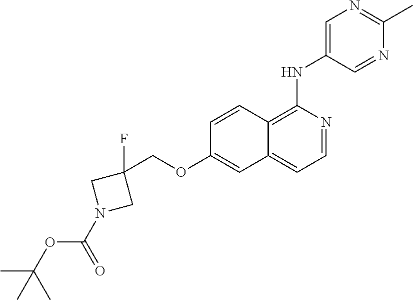 [SMILES: Cc1ncc(Nc2nccc3cc(OCC4(F)CN(C(=O)OC(C)(C)C)C4)ccc23)cn1]

To a mixture of tert-butyl 3-(((1-chloroisoquinolin-6-yl)oxy)methyl)-3-fluoroazetidine-1-carboxylate (0.500 g, 1.36 mmol), 2-methylpyrimidin-5-amine (0.179 g, 1.64 mmol), and potassium carbonate (0.564 g, 4.08 mmol) in 1,4-dioxane (10 mL) was added (2-dicyclohexylphosphino-2′,6′-diisopropoxy-1,1′-biphenyl)[2-(2′-amino-1,1′-biphenyl)]palladium(II) methanesulfonate (0.114 g, 0.136 mmol) and the mixture was stirred at 100° C. for 12 h. After cooling to ambient temperature, the mixture was diluted with ethyl acetate (20 mL) and washed with saturated sodium bicarbonate solution (3×20 mL). The organic phase was dried over anhydrous sodium sulfate, filtered, and the filtrate was concentrated under reduced pressure. The obtained residue was purified by reverse-phase column chromatography, eluting with acetonitrile in water (containing 0.1% of formic acid) to the title compound as a colorless solid (0.235 g, 85% purity): ¹H NMR (400 MHz, DMSO-d₆) 59.32 (s, 1H), 9.21-9.12 (m, 2H), 8.45 (d, J=10.0 Hz, 1H), 7.96 (d, J=5.6 Hz, 1H), 7.38-7.27 (m, 2H), 7.15 (d, J=6.0 Hz, 1H), 4.62-4.50 (m, 2H), 4.25-4.00 (m, 4H), 2.57 (s, 3H), 1.40 (s, 9H).

### Step 4. Preparation of 6-((3-fluoroazetidin-3-yl)methoxy)-N-(2-methylpyrimidin-5-yl)isoquinolin-1-amine

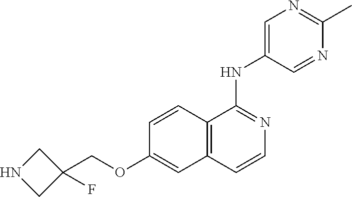 [SMILES: Cc1ncc(Nc2nccc3cc(OCC4(F)CNC4)ccc23)cn1]

To a mixture of tert-butyl 3-fluoro-3-(((1-((2-methylpyrimidin-5-yl)amino)isoquinolin-6-yl)oxy)methyl)azetidine-1-carboxylate (0.230 g, 0.523 mmol) in 1,4-dioxane (1 mL) was added a 4 M solution of hydrogen chloride in 1,4-dioxane (5 mL, 20 mmol) and the mixture was stirred at ambient temperature for 1 h. The mixture was concentrated under reduced pressure and the residue was poured into saturated sodium carbonate solution (50 mL). The mixture was extracted with ethyl acetate (3×50 mL). The combined organic layers were washed with brine (50 mL), dried over anhydrous sodium sulfate, and filtered. The filtrate was concentrated under reduced pressure to give a residue (0.130 g). A portion of the residue (0.050 g) was purified by reverse-phase preparative HPLC (Waters XBridge 150 mm×25 mm, 5 μm column), eluting with a gradient of 16 to 48% of acetonitrile in water (containing ammonium hydroxide), to give the title compound as a colorless solid (0.023 g, 30% yield): ¹H NMR (400 MHz, DMSO-d₆) 59.33 (s, 1H), 9.16 (s, 2H), 8.45 (d, J=9.2 Hz, 1H), 7.96 (d, J=5.6 Hz, 1H), 7.40-7.29 (m, 2H), 7.16 (d, J=5.6 Hz, 1H), 4.58-4.42 (m, 2H), 3.70 (dd, J=10.0, 19.6 Hz, 2H), 3.60-3.48 (m, 2H), 2.57 (s, 3H), 1.83 (s, 1H); MS (ES+) m/z 340.2 (M+1).

### Example 59

### Synthesis of N-(6-chloropyridin-3-yl)-6-((3-fluoroazetidin-3-yl)methoxy)isoquinolin-1-amine hydrochloride

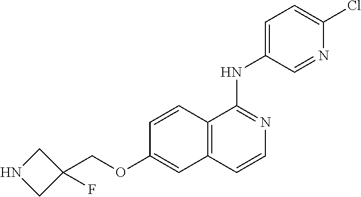 [SMILES: FC1(COc2ccc3c(Nc4ccc(Cl)nc4)nccc3c2)CNC1]

### Step 1. Preparation of tert-butyl 3-(((1-((6-chloropyridin-3-yl)amino)isoquinolin-6-yl)oxy)methyl)-3-fluoroazetidine-1-carboxylate

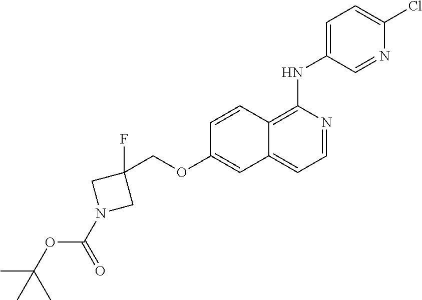 [SMILES: CC(C)(C)OC(=O)N1CC(F)(COc2ccc3c(Nc4ccc(Cl)nc4)nccc3c2)C1]

Following the procedure as described for EXAMPLE 58, Step 3, and making variations as required to replace 2-methylpyrimidin-5-amine with 6-chloropyridin-3-amine, the title compound was obtained as a colorless solid (0.075 g, 27% yield): ¹H NMR (400 MHz, CDCl₃) δ 9.40 (s, 1H), 8.88 (d, J=2.8 Hz, 1H), 8.47 (d, J=8.8 Hz, 1H), 8.42 (dd, J=8.8, 2.8 Hz, 1H), 7.98 (d, J=5.6 Hz, 1H), 7.44 (d, J=8.8 Hz, 1H), 7.36-7.29 (m, 2H), 7.17 (d, J=5.6 Hz, 1H), 4.62-4.47 (m, 2H), 4.27-3.98 (m, 4H), 1.40 (s, 9H).

### Step 2. Preparation of N-(6-chloropyridin-3-yl)-6-((3-fluoroazetidin-3-yl)methoxy)isoquinolin-1-amine hydrochloride

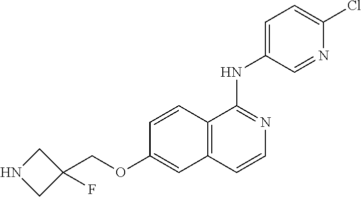 [SMILES: FC1(COc2ccc3c(Nc4ccc(Cl)nc4)nccc3c2)CNC1]

To a mixture of tert-butyl 3-(((1-((6-chloropyridin-3-yl)amino)isoquinolin-6-yl)oxy)methyl)-3-fluoroazetidine-1-carboxylate (0.036 g, 0.078 mmol) in dichloromethane (1 mL) was added a 4 M solution of hydrogen chloride in ethyl acetate (1 mL, 4.0 mmol) and the mixture was stirred at ambient temperature for 1 h. The mixture was then concentrated under reduced pressure and the residue was lyophilized to the title compound as a colorless solid (0.018 g, 61% yield): ¹H NMR (400 MHz, DMSO-d₆) δ 9.78 (s, 1H), 9.63 (s, 1H), 8.79 (d, J=10.0 Hz, 1H), 8.72 (d, J=2.4 Hz, 1H), 8.65-8.60 (m, 1H), 8.17 (d, J=8.4 Hz, 1H), 7.69 (t, J=9.6 Hz, 2H), 7.61-7.48 (m, 2H), 7.33 (d, J=6.7 Hz, 1H), 4.79-4.65 (m, 2H), 4.42-4.21 (m, 4H); ¹⁹F NMR (376 MHz, DMSO-d₆) 5-150.9 (s); MS (ES+) m/z 359.1 (M+1), 361.1 (M+1).

### Example 60

### Synthesis of 1-((1-((2-chloropyrimidin-5-yl)amino)isoquinolin-6-yl)oxy)cyclopropane-1-carbonitrile

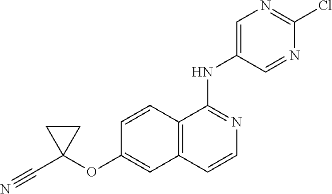 [SMILES: N#CC1(Oc2ccc3c(Nc4cnc(Cl)nc4)nccc3c2)CC1]

### Step 1. Preparation of methyl 4-bromo-2-((1-chloroisoquinolin-6-yl)oxy)butanoate

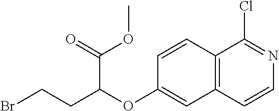 [SMILES: COC(=O)C(CCBr)Oc1ccc2c(Cl)nccc2c1]

To a solution of 1-chloroisoquinolin-6-ol (1.00 g, 5.57 mmol) in N,N-dimethyl formamide (12 mL) was added methyl 2,4-dibromobutanoate (1.88 g, 7.24 mmol) and potassium carbonate (2.32 g, 16.75 mmol) and the mixture was stirred at ambient temperature for 4 h. The reaction mixture was diluted with water (30 mL) and extracted with ethyl acetate (3×20 mL). The combined organic phase was washed with brine (3×10 mL), dried over anhydrous sodium sulfate, and filtered. The filtrate was concentrated in vacuo to provide a residue which was purified by silica gel column chromatography, eluting with a gradient of 0 to 50% of ethyl acetate in petroleum ether, to afford the title compound as a yellowish solid (1.40 g, 49% yield): ¹H NMR (400 MHz, DMSO-d₆) δ 8.23-8.19 (m, 2H), 7.78 (d, J=5.6 Hz, 1H), 7.50 (dd, J=9.2, 2.4 Hz, 1H), 7.46 (d, J=2.4 Hz, 1H), 5.27 (dd, J=4.8, 7.6 Hz, 1H), 3.76-3.65 (m, 5H), 2.55-2.51 (m, 2H).

### Step 2. Preparation of 1-((1-chloroisoquinolin-6-yl)oxy)cyclopropane-1-carboxylic acid

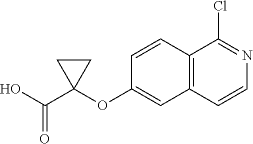 [SMILES: O=C(O)C1(Oc2ccc3c(Cl)nccc3c2)CC1]

To a solution of methyl 4-bromo-2-((1-chloroisoquinolin-6-yl)oxy)butanoate (1.30 g, 2.54 mmol) in tetrahydrofuran (20 mL) was added potassium tert-butoxide (0.850 g, 7.57 mmol) at −10° C. The mixture was allowed to warm up to ambient temperature and stirred at this temperature for 16 h. The reaction mixture was diluted with water (30 mL) and extracted with ethyl acetate (3×20 mL). The aqueous layer was acidified to pH=2 with 1 M hydrochloric acid and extracted with ethyl acetate (3×20 mL). The combined organic phase was washed with brine (3×10 mL), dried over anhydrous sodium sulfate, and filtered. The filtrate was concentrated in vacuo to afford the title compound as a yellowish solid (0.750 g, 96% yield): ¹H NMR (400 MHz, DMSO-d₆) δ 13.12 (s, 1H), 8.22-8.18 (m, 2H), 7.85 (d, J=5.6 Hz, 1H), 7.48 (d, J=2.4 Hz, 1H), 7.43 (dd, J=9.2, 2.4 Hz, 1H), 1.65-1.60 (m, 2H), 1.41-0.35 (m, 2H).

### Step 3. Preparation of 1-((1-chloroisoquinolin-6-yl)oxy)cyclopropane-1-carboxamide

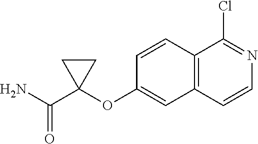 [SMILES: NC(=O)C1(Oc2ccc3c(Cl)nccc3c2)CC1]

To a solution of 1-((1-chloroisoquinolin-6-yl)oxy)cyclopropane-1-carboxylic acid (0.100 g, 0.326 mmol) in N,N-dimethylformamide (2 mL) was added ammonium chloride (0.023 g, 0.430 mmol), 0-(7-azabenzotriazol-1-yl)-N,N,N′,N′-tetramethyluronium hexafluorophosphate (0.149 g, 0.392 mmol) and N,N-diisopropylethylamine (0.17 mL, 0.976 mmol). The mixture was stirred at ambient temperature for 16 h and then diluted with water (20 mL) and extracted with ethyl acetate (3×10 mL). The combined organic phase was washed with brine (3×10 mL), dried over anhydrous sodium sulfate, and filtered. The filtrate was concentrated in vacuo to provide a residue which was purified by silica gel column chromatography, eluting with 0 to 50% ethyl acetate in petroleum ether, to afford the title compound as a yellowish solid (0.076 g, 81% yield): ¹H NMR (400 MHz, DMSO-d₆) δ 8.24-8.19 (m, 2H), 7.86 (d, J=5.6 Hz, 1H), 7.57 (s, 1H), 7.43 (d, J=2.4 Hz, 2H), 7.37 (s, 1H), 1.55-1.49 (m, 2H), 1.22-1.18 (m, 2H).

### Step 4. Preparation of 1-((1-chloroisoquinolin-6-yl)oxy)cyclopropane-1-carbonitrile

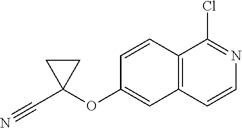 [SMILES: N#CC1(Oc2ccc3c(Cl)nccc3c2)CC1]

To a solution of 1-((1-chloroisoquinolin-6-yl)oxy)cyclopropane-1-carboxamide (0.0700 g, 0.242 mmol) and pyridine (0.060 mL, 0.743 mmol) in dichloromethane (2 mL) was added trifluoroacetic anhydride (0.130 mL, 0.935 mmol) dropwise at 0° C. The reaction mixture was then heated to 60° C. for 1 h. After cooling to ambient temperature, the reaction mixture was diluted with water (10 mL) and extracted with ethyl acetate (3×10 mL). The combined organic phase was washed with brine (3×5 mL), dried over anhydrous sodium sulfate, and filtered. The filtrate was concentrated in vacuo to provide the title compound as a colorless solid (0.060 g, 91% yield): ¹H NMR (400 MHz, DMSO-d₆) δ 8.31-8.27 (m, 2H), 7.97 (d, J=5.6 Hz, 1H), 7.87 (d, J=2.4 Hz, 1H), 7.52 (dd, J=9.2, 2.4 Hz, 1H), 1.83-1.79 (m, 2H), 1.63-1.58 (m, 2H).

### Step 5. Preparation of 1-((1-((2-chloropyrimidin-5-yl)amino)isoquinolin-6-yl)oxy)cyclopropane-1-carbonitrile

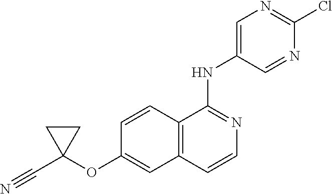 [SMILES: N#CC1(Oc2ccc3c(Nc4cnc(Cl)nc4)nccc3c2)CC1]

To a solution of 1-((1-chloroisoquinolin-6-yl)oxy)cyclopropane-1-carbonitrile (0.040 g, 0.147 mmol) and 2-chloropyrimidin-5-amine (0.0200 g, 0.154 mmol) in 2-methylbutan-2-ol (4 mL) was added (2-dicyclohexylphosphino-2′,6′-diisopropoxy-1,1′-biphenyl)[2-(2′-amino-1,1′-biphenyl)]palladium(II) methanesulfonate (0.013 g, 0.0154 mmol) and cesium carbonate (0.145 g, 0.445 mmol) and the mixture was stirred at ambient temperature for 16 h. The reaction mixture was diluted with water (10 mL) and extracted with ethyl acetate (3×10 mL). The combined organic phase was washed with brine (3×5 mL), dried over anhydrous sodium sulfate, and filtered. The filtrate was concentrated in vacuo to provide a residue was purified by reverse-phase preparative HPLC (Phenomenex Luna C18 150 mm×25 mm, 10 μm column), eluting with a gradient of 36 to 56% of acetonitrile in water (containing 0.5% of formic acid), to afford the title compound as a colorless solid (0.009 g, 18% yield): ¹H NMR (400 MHz, DMSO-d₆) δ 9.67 (s, 1H), 9.29 (s, 2H), 8.53 (d, J=9.2 Hz, 1H), 8.07 (d, J=6.0 Hz, 1H), 7.67 (d, J=2.4 Hz, 1H), 7.43-7.39 (m, 2H), 1.83-1.78 (m, 2H), 1.61-1.56 (m, 2H); MS (ES+) m/z 338.1 (M+1), 340.1 (M+1).

### Example 61

### Synthesis of 1-((1-((2-chloropyrimidin-5-yl)amino)isoquinolin-6-yl)oxy)cyclopropane-1-carboxamide

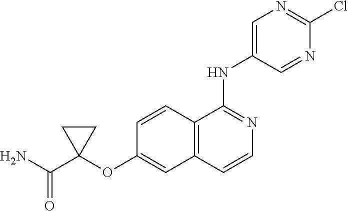 [SMILES: NC(=O)C1(Oc2ccc3c(Nc4cnc(Cl)nc4)nccc3c2)CC1]

To a solution of 2-chloropyrimidin-5-amine (0.025 g, 0.190 mmol) and 1-((1-chloroisoquinolin-6-yl)oxy)cyclopropane-1-carboxamide (0.058 g, 0.190 mmol) in 2-methylbutan-2-ol (2 mL) was added (2-dicyclohexylphosphino-2′,6′-diisopropoxy-1,1′-biphenyl)[2-(2′-amino-1,1′-biphenyl)]palladium(II) methanesulfonate (0.186 g, 0.571 mmol) and cesium carbonate (0.186 g, 0.571 mmol), and the mixture was heated to 30° C. for 5 h. After cooling to ambient temperature, the reaction mixture was diluted with water (10 mL) and extracted with ethyl acetate (3×10 mL). The combined organic phase was washed with brine (3×5 mL), dried over anhydrous sodium sulfate, and filtered. The filtrate was concentrated in vacuo to provide a residue which was purified by reverse-phase preparative HPLC (Phenomenex Luna C18 150 mm×25 mm, 10 μm column), eluting with a gradient of 12 to 42% of acetonitrile in water (containing 0.5% of formic acid), to afford the title compound as a yellow solid (0.006 g, 7% yield): ¹H NMR (400 MHz, DMSO-d₆) 59.60 (s, 1H), 9.29 (s, 2H), 8.45 (d, J=8.8 Hz, 1H), 8.00 (d, J=5.6 Hz, 1H), 7.53 (s, 1H), 7.37 (s, 1H), 7.31-7.26 (m, 2H), 7.23 (d, J=2.4 Hz, 1H), 1.54-1.48 (m, 2H), 1.20-1.15 (m, 2H); MS (ES+) m/z 356.2 (M+1), 358.2 (M+1).

### Example 62

### Synthesis of 3-(((1-((6-chloropyridin-3-yl)amino)isoquinolin-6-yl)oxy)methyl)-1-methyl-1H-pyrazole-5-carbonitrile

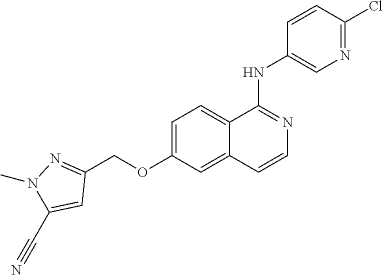 [SMILES: Cn1nc(COc2ccc3c(Nc4ccc(Cl)nc4)nccc3c2)cc1C#N]

### Step 1. Preparation of (3-(bromomethyl)-1-methyl-1H-pyrazole-5-carbonitrile

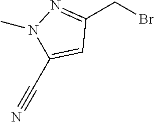

To a solution of 1,3-dimethyl-1H-pyrazole-5-carbonitrile (0.300 g, 2.48 mmol) in chloroform-d (10 mL) was added N-bromosuccinimide (0.441 g, 2.48 mmol) and azobisisobutyronitrile (0.041 g, 0.248 mmol) and the reaction mixture was stirred at 85° C. for 2 h. After cooling to ambient temperature, the reaction mixture was concentrated in vacuo. The residue was purified by reverse-phase column chromatography, eluting with acetonitrile in water containing 0.1% of formic acid, to provide the title compound as a brownish oil (0.200 g, 39% yield): MS (ES+) m/z 199.9 (M+1), 201.9 (M+1).

### Step 2. Preparation of 3-(((1-((6-chloropyridin-3-yl)amino)isoquinolin-6-yl)oxy)methyl)-1-methyl-1H-pyrazole-5-carbonitrile

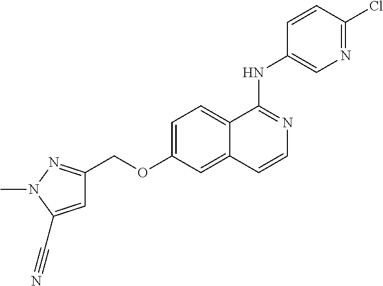 [SMILES: Cn1nc(COc2ccc3c(Nc4ccc(Cl)nc4)nccc3c2)cc1C#N]

To a solution of 3-(bromomethyl)-1-methyl-1H-pyrazole-5-carbonitrile (0.067 g, 0.334 mmol) in N,N-dimethylformamide (1 mL) was added potassium carbonate (0.115 g, 0.835 mmol) and 1-chloroisoquinolin-6-ol (0.050 g, 0.278 mmol) at ambient temperature. The reaction mixture was heated up to 90° C. for 12 h. After cooling to ambient temperature, the reaction mixture was poured into water (10 mL). The mixture was extracted with ethyl acetate (3×10 mL). The combined organic phase was washed with brine (10 mL), dried over anhydrous sodium sulfate, and filtered. The filtrate was concentrated in vacuo and the obtained residue was purified by silica gel column chromatography, eluting with 50% of ethyl acetate in petroleum ether, to provide a colorless solid (0.066 g). To the residue was added 6-chloropyridin-3-amine (0.028 g, 0.221 mmol), [(2-di-tert-butylphosphino-2′,4′,6′-triisopropyl-1,1′-biphenyl)-2-(2′-amino-1,1′-biphenyl)]palladium(II) methanesulfonate (0.018 g, 0.0221 mmol), cesium carbonate (0.216 g, 0.663 mmol) and 1,4-dioxane (2 mL). The reaction mixture was then stirred at 90° C. for 12 h.

After cooling to ambient temperature, the reaction mixture was poured into water (20 mL). The mixture was extracted with ethyl acetate (3×20 mL). The combined organic phase was washed with brine (10 mL), dried over anhydrous sodium sulfate, and filtered. The filtrate was concentrated in vacuo and the obtained residue was purified by silica gel column chromatography, eluting with a gradient of 80% to 100% of ethyl acetate in petroleum ether. The residue was then purified by reverse-phase preparative HPLC (Waters XBridge 150 mm×25 mm, 5 μm column), eluting with a gradient of 46% to 76% of acetonitrile in water (containing 10 mM of ammonium carbonate), to provide the title compound as a colorless solid (0.014 g, 13% yield): ¹H NMR (400 MHz, DMSO-d₆) δ 9.40 (s, 1H), 8.87 (d, J=2.8 Hz, 1H), 8.46-8.40 (m, 2H), 7.97 (d, J=5.8 Hz, 1H), 7.45-7.42 (m, 2H), 7.32-7.29 (m, 2H), 7.19 (d, J=5.9 Hz, 1H), 5.23 (s, 2H), 4.03 (s, 3H); MS (ES+) m/z 391.1 (M+1), 393.1 (M+1).

### Example 63

### Synthesis of N-(6-chloropyridin-3-yl)-6-((5-methyl-1,3,4-oxadiazol-2-yl)methoxy)isoquinolin-1-amine

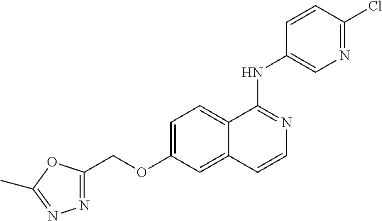 [SMILES: Cc1nnc(COc2ccc3c(Nc4ccc(Cl)nc4)nccc3c2)o1]

To a solution of 2-(chloromethyl)-5-methyl-1,3,4-oxadiazole (0.295 g, 2.23 mmol) in N,N-dimethylformamide (4 mL) was added potassium carbonate (0.462 g, 3.34 mmol) and 1-chloroisoquinolin-6-ol (0.200 g, 1.11 mmol) and the reaction mixture was heated to 90° C. for 12 h. After cooling to ambient temperature, the reaction mixture was poured into water (50 mL). The mixture was extracted with ethyl acetate (3×50 mL). The combined organic phase was washed with brine (50 mL), dried over anhydrous sodium sulfate, and filtered. The filtrate was concentrated in vacuo to provide a residue (0.210 g). To the residue was added 6-chloropyridin-3-amine (0.118 g, 0.914 mmol), [(2-di-tert-butylphosphino-2′,4′,6′-triisopropyl-1,1′-biphenyl)-2-(2′-amino-1,1′-biphenyl)]palladium(II) methanesulfonate (0.061 g, 0.076 mmol), cesium carbonate (0.745 g, 2.29 mmol), and 2-methylbutan-2-ol (10 mL) and the mixture was stirred at 90° C. for 12 h. After cooling to ambient temperature, the reaction mixture was poured into water (40 mL). The mixture was extracted with ethyl acetate (3×40 mL). The combined organic phase was washed with brine (40 mL), dried over anhydrous sodium sulfate, and filtered. The filtrate was concentrated in vacuo and the obtained residue was purified by reverse-phase preparative HPLC (Phenomenex Synergi C18 150 mm×25 mm, 10 μm column), eluting with a gradient of 9% to 39% of acetonitrile in water (containing 0.225% of formic acid), to provide the title compound as a yellowish solid (0.037 g, 9% yield): ¹H NMR (400 MHz, DMSO-d₆) 59.45-9.44 (m, 1H), 8.88 (d, J=2.8 Hz, 1H), 8.49 (d, J=9.3 Hz, 1H), 8.42 (dd, J=8.7, 2.8 Hz, 1H), 7.99 (d, J=5.8 Hz, 1H), 7.46-7.44 (m, 2H), 7.37 (dd, J=9.2, 2.6 Hz, 1H), 7.18 (d, J=5.8 Hz, 1H), 5.54 (s, 2H) 2.54 (s, 3H); MS (ES+) m/z 368.1 (M+1), 370.1 (M+1).

### Example 64

### Synthesis of (1s,3s)-3-(((1-((2-chloropyrimidin-5-yl)amino)isoquinolin-6-yl)oxy)methyl)-3-fluorocyclobutane-1-carbonitrile

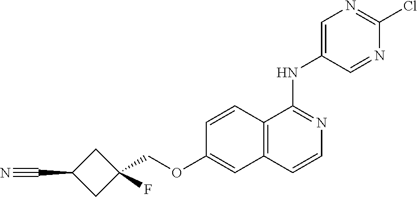 [SMILES: N#C[C@H]1C[C@](F)(COc2ccc3c(Nc4cnc(Cl)nc4)nccc3c2)C1]

### Step 1. Preparation of 3-(bromomethyl)-3-hydroxycyclobutane-1-carbonitrile

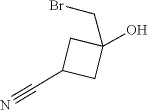

To a solution of 3-methylenecyclobutane-1-carbonitrile (5.00 g, 53.7 mmol) in water (20 mL) and 1,4-dioxane (20 mL) was added N-bromosuccinimide (9.56 g, 53.7 mmol) at 0° C. The reaction mixture was stirred at ambient temperature for 12 h and then concentrated in vacuo. The residue was purified by silica gel column chromatography, eluting with a gradient of 30% to 100% of ethyl acetate in petroleum ether, to provide the title compound as a colorless oil (1.90 g, 19% yield).

### Step 2. Preparation of 3-(((1-chloroisoquinolin-6-yl)oxy)methyl)-3-hydroxycyclobutane-1-carbonitrile

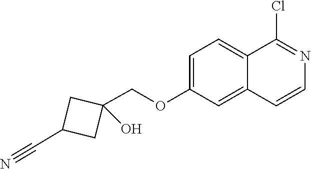 [SMILES: N#CC1CC(O)(COc2ccc3c(Cl)nccc3c2)C1]

A solution of 3-(bromomethyl)-3-hydroxycyclobutane-1-carbonitrile (1.50 g, 7.89 mmol), 1-chloroisoquinolin-6-ol (0.709 mg, 3.95 mmol), and potassium carbonate (2.18 g, 15.6 mmol) in N,N-dimethylformamide (12 mL) was heated to 80° C. for 12 h. After cooling to ambient temperature, the reaction mixture was diluted with water (25 mL) and extracted with ethyl acetate (2×30 mL). The combined organic phase was washed with brine (25 mL), dried over anhydrous sodium sulfate, and filtered. The filtrate was concentrated in vacuo and the obtained residue was purified by silica gel column chromatography, eluting with a gradient of 50 to 100% of ethyl acetate in petroleum ether, to provide the title compound as a colorless solid (0.680 g, 30% yield): MS (ES+) m/z 289.1 (M+1), 291.1 (M+1).

### Step 3. Preparation of 3-(((1-chloroisoquinolin-6-yl)oxy)methyl)-3-fluorocyclobutane-1-carbonitrile

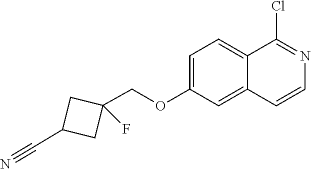 [SMILES: N#CC1CC(F)(COc2ccc3c(Cl)nccc3c2)C1]

To a solution of 3-(((1-chloroisoquinolin-6-yl)oxy)methyl)-3-hydroxycyclobutane-1-carbonitrile (0.680 g, 2.36 mmol) in dichloromethane (10 mL) was added diethylaminosulfur trifluoride (0.759 g, 4.71 mmol) at 0° C. The reaction mixture was allowed to warm to ambient temperature and stirred for 12 h. The reaction mixture was diluted with saturated sodium bicarbonate solution (15 mL) and extracted with dichloromethane (2×10 mL). The combined organic phase was washed with brine (15 mL), dried over anhydrous sodium sulfate, and filtered. The filtrate was concentrated in vacuo and the obtained residue was purified by silica gel column chromatography, eluting with a gradient of 50 to 100% of ethyl acetate in petroleum ether, to provide the title compound as a colorless solid (0.460 g, 67% yield): MS (ES+) m/z 291.1 (M+1), 293.1 (M+1).

### Step 4. Preparation of (1s,3s)-3-(((1-((2-chloropyrimidin-5-yl)amino)isoquinolin-6-yl)oxy)methyl)-3-fluorocyclobutane-1-carbonitrile

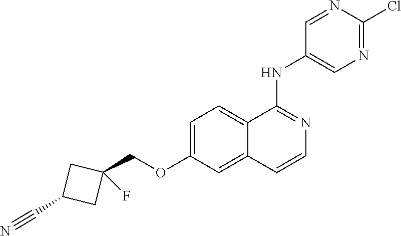 [SMILES: N#C[C@H]1C[C@](F)(COc2ccc3c(Nc4cnc(Cl)nc4)nccc3c2)C1]

A mixture of 3-(((1-chloroisoquinolin-6-yl)oxy)methyl)-3-fluorocyclobutane-1-carbonitrile (0.460 g, 1.58 mmol), 2-chloropyrimidin-5-amine (0.205 g, 1.58 mmol), [(2-di-tert-butylphosphino-2′,4′,6′-triisopropyl-1,1′-biphenyl)-2-(2′-amino-1,1′-biphenyl)] palladium(II) methanesulfonate (0.132 g, 0.158 mmol), and cesium carbonate (1.55 g, 4.75 mmol) in 2-methylbutan-2-ol (12 mL) was stirred at 45° C. for 2 h. After cooling to ambient temperature, the reaction mixture was concentrated in vacuo. The residue was purified by reverse-phase preparative HPLC (Phenomenex Luna C18 150 mm×25 mm, 10 μm column), eluting with a gradient of 28% to 58% of acetonitrile in water (containing 0.225% of formic acid), to provide the title compound as a yellow solid (0.013 g, 2% yield). The relative configuration of the title compound was assigned based on 2D-NOESY NMR. ¹H NMR (400 MHz, DMSO-d₆) 59.59 (s, 1H), 9.30 (d, J=3.4 Hz, 2H), 8.46 (d, J=9.0 Hz, 1H), 8.02 (d, J=5.8 Hz, 1H), 7.40-7.35 (m, 2H), 7.24 (d, J=5.8 Hz, 1H), 4.39 (d, J=23.3 Hz, 2H), 3.27-3.20 (m, 1H), 2.87-2.66 (m, 4H); ¹⁹F NMR (376 MHz, DMSO-d₆) 5-135.4 (s); MS (ES+) m/z 384.2 (M+1), 386.2 (M+1).

### Example 65

### Synthesis of N-(6-chloropyridin-3-yl)-6-((1-methyl-1H-imidazol-5-yl)methoxy)isoquinolin-1-amine

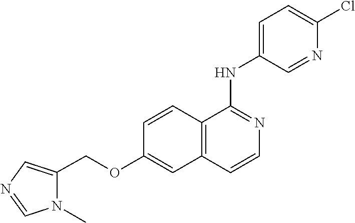 [SMILES: Cn1cncc1COc1ccc2c(Nc3ccc(Cl)nc3)nccc2c1]

A solution of 1-chloroisoquinolin-6-ol (0.180 g, 1.38 mmol), 5-(chloromethyl)-1-methyl-1H-imidazole (0.246 g, 1.38 mmol), and cesium carbonate (0.900 g, 2.76 mmol) in N,N-dimethylformamide (6 mL) was stirred at 90° C. for 12 h. After cooling to ambient temperature, water (15 mL) and ethyl acetate (15 mL) were added to the reaction mixture. The mixture was extracted with ethyl acetate (2×15 mL). The combined organic phase was washed with brine (10 mL), dried over anhydrous sodium sulfate, and filtered. The filtrate was concentrated in vacuo and the residue was purified by silica gel column chromatography, eluting with 25% of methanol in ethyl acetate, to provide a colorless solid (0.060 g). To the obtained residue was added 6-chloropyridin-3-amine (0.043 g, 0.335 mmol) and propan-2-ol (2 mL), followed by a 4 M solution of hydrogen chloride in dioxane (0.20 mL, 0.800 mmol). The reaction mixture was stirred at 70° C. for 12 h. After cooling to ambient temperature, the mixture was diluted with water (15 mL) and extracted with ethyl acetate (3×15 mL). The combined organic phase was washed with brine (10 mL), dried over anhydrous sodium sulfate, and filtered. The filtrate was concentrated in vacuo and the obtained residue was by silica gel column chromatography, eluting with 25% of methanol in ethyl acetate. The residue was then purified by reverse-phase preparative HPLC (Waters Xbridge 150 mm×25 mm, 5 μm column), eluting with a gradient of 26% to 56% of acetonitrile in water (containing 0.1% of ammonium hydroxide), to provide the title compound as a colorless solid (0.011 g, 2% yield): ¹H NMR (400 MHz, DMSO-d₆) 59.38 (s, 1H), 8.88 (d, J=2.8 Hz, 1H), 8.52-8.38 (m, 2H), 7.98 (d, J=5.6 Hz, 1H), 7.68 (s, 1H), 7.48-7.42 (m, 2H), 7.32 (dd, J=2.4, 9.2 Hz, 1H), 7.21 (d, J=6.0 Hz, 1H), 7.11 (s, 1H), 5.27 (s, 2H), 3.68 (s, 3H); MS (ES+) m/z 366.0 (M+1), 368.0 (M+1).

### Example 66

### Synthesis of N-(2-chloropyrimidin-5-yl)-6-(2,2-difluoroethoxy)isoquinolin-1-amine

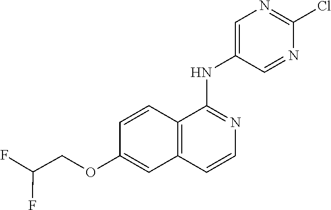 [SMILES: FC(F)COc1ccc2c(Nc3cnc(Cl)nc3)nccc2c1]

### Step 1. Preparation of 1-chloro-6-(2,2-difluoroethoxy)isoquinoline

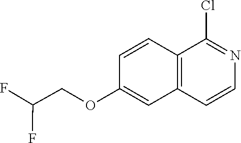 [SMILES: FC(F)COc1ccc2c(Cl)nccc2c1]

To a solution of 1-chloroisoquinolin-6-ol (0.250 g, 1.39 mmol), 2,2-difluoroethanol (0.171 g, 2.08 mmol), and triphenylphosphine (0.547 g, 2.09 mmol) in tetrahydrofuran (14 mL) was slowly added diisopropyl azodicarboxylate (0.41 mL, 2.08 mmol) at 0° C. The reaction mixture was allowed to warm to ambient temperature and stirred for 24 h, and then concentrated in vacuo. The residue was purified by silica gel column chromatography, eluting with a gradient of 0% to 30% of ethyl acetate in heptane, to afford the title compound as a colorless solid (0.270 g, 80% yield): MS (ES+) m/z 244.6 (M+1), 246.0 (M+1).

### Step 2. Preparation of N-(2-chloropyrimidin— 5-yl)₆-(2,2-difluoroethoxy)isoquinolin-1-amine

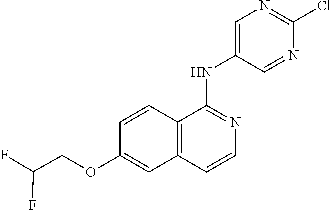 [SMILES: FC(F)COc1ccc2c(Nc3cnc(Cl)nc3)nccc2c1]

A mixture of 1-chloro-6-(2,2-difluoroethoxy)isoquinoline (0.109 g, 0.447 mmol), 5-amino-2-chloropyrimidine (0.058 mg, 0.447 mmol), and potassium phosphate tribasic (0.285 g, 1.34 mmol) in 1,2-dimethoxyethane (5 mL) was purged with argon for 20 minutes. To it was then added 2-dicyclohexylphosphino-2′,4′,6′-tri-isopropyl-1,1′-biphenyl (0.021 g, 0.045 mmol), followed by tris(dibenzylideneacetone)dipalladium(0) (0.021 g, 0.022 mmol), and the mixture was purged with argon for additional 5 minutes. The reaction mixture was then stirred at 110° C. for 16 h. After cooling to ambient temperature, the mixture was filtered through a pad of diatomaceous earth. The pad was washed with ethyl acetate (2×20 mL) and the combined filtrate was concentrated in vacuo. The residue was purified by silica gel column chromatography, eluting with a gradient of 0% to 50% of ethyl acetate in heptane, to afford the title compound as a colorless solid (0.027 g, 18% yield): ¹H NMR (300 MHz, DMSO-d₆) 59.61 (s, 1H), 9.29 (s, 2H), 8.46 (d, J=9.0 Hz, 1H), 8.02 (d, J=5.8 Hz, 1H), 7.45-7.37 (m, 2H), 7.25 (d, J=5.6 Hz, 1H), 6.49 (tt, J=54.4, 3.5 Hz, 1H), 4.50 (td, J=14.6, 3.6 Hz, 2H); MS (ES+) m/z 337.0 (M+1), 339.0 (M+1).

### Example 67

### Synthesis of N-(6-methoxypyridin-3-yl)-6-((3-methyloxetan-3-yl)methoxy)isoquinolin-1-amine

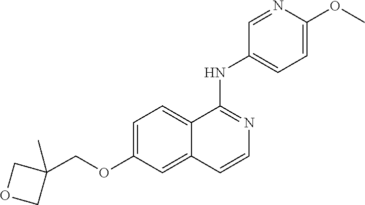 [SMILES: COc1ccc(Nc2nccc3cc(OCC4(C)COC4)ccc23)cn1]

Following the procedure as described for EXAMPLE 36, Step 3, and making variations as required to replace 6-chloro-5-methoxypyridin-3-amine with 5-amino-2-methoxypyridine, the title compound was obtained as a colorless solid (0.0036 g, 3% yield): ¹H NMR (400 MHz, CDCl₃) δ 8.28 (d, J=2.7 Hz, 1H), 8.00 (dt, J=12.6, 4.7 Hz, 2H), 7.86 (d, J=9.1 Hz, 1H), 7.21 (dd, J=9.1, 2.4 Hz, 1H), 7.08 (d, J=2.3 Hz, 1H), 7.02 (d, J=5.8 Hz, 1H), 6.80 (d, J=8.9 Hz, 1H), 4.66 (d, J=6.0 Hz, 2H), 4.51 (d, J=6.0 Hz, 2H), 4.17 (s, 2H), 3.94 (s, 3H), 1.49 (s, 3H), NH not observed; MS (ES+) m/z 352.0 (M+1).

### Example 68

### Synthesis of N-(2-chloropyrimidin-5-yl)-6-((1-fluorocyclopropyl)methoxy)isoquinolin-1-amine

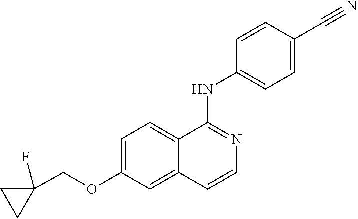 [SMILES: N#Cc1ccc(Nc2nccc3cc(OCC4(F)CC4)ccc23)cc1]

### Step 1. Preparation of (1-fluorocyclopropyl)methanol

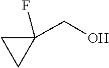

To a solution of 1-fluoro-cyclopropanecarboxylic acid (1.00 g, 9.61 mmol) in anhydrous tetrahydrofuran (20 mL) was added lithium aluminum hydride (1.0 M solution in tetrahydrofuran, 14.0 mL, 14.4 mmol) at 0° C. The reaction mixture was stirred at 0° C. for 30 minutes and then heated to reflux for 2 h. The reaction was cooled to 0° C. and quenched by portionwise addition of 3.0 g of sodium sulfate decahydrate. The mixture was stirred at ambient temperature for 16 h.

The solid was filtered off and the filtrate was concentrated in vacuo to afford the title compound as a colorless oil (0.850 g, 98% yield): ¹H NMR (400 MHz, CDCl₃) δ3.79 (d, J=22.1 Hz, 2H), 1.09-1.01 (m, 2H), 0.70-0.64 (m, 2H), OH not observed.

### Step 2. Preparation of (1-fluorocyclopropyl)methyl 4-methylbenzenesulfonate

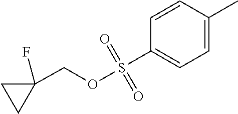 [SMILES: Cc1ccc(S(=O)(=O)OCC2(F)CC2)cc1]

To a solution of (1-fluorocyclopropyl)methanol (0.850 g, 9.43 mmol) in dichloromethane (10 mL) was added p-toluenesulfonyl chloride (2.698 g, 14.2 mmol) and triethylamine (3.90 mL, 28.3 mmol). The reaction mixture was stirred at ambient temperature for 16 h. The reaction mixture was diluted with dichloromethane (30 mL), washed with water (20 mL), and saturated ammonium chloride (30 mL). The organic phase was dried over anhydrous sodium sulfate, filtered, and the filtrate was concentrated in vacuo to afford the title compound as a colorless oil (1.60 g, 69% yield): ¹H NMR (400 MHz, CDCl₃) δ7.80 (d, J=8.3 Hz, 2H), 7.34 (d, J=8.0 Hz, 2H), 4.26 (d, J=21.6 Hz, 2H), 2.44 (s, 3H), 1.15-1.07 (m, 2H), 0.76-0.70 (m, 2H).

### Step 3. Preparation of 1-chloro-6-((1-fluorocyclopropyl)methoxy)isoquinoline

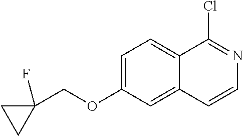 [SMILES: FC1(COc2ccc3c(Cl)nccc3c2)CC1]

To a solution of (3-methyloxetan-3-yl)methyl 4-methylbenzenesulfonate (0.550 g, 2.25 mmol) in N,N-dimethylformamide (6 mL), was added potassium carbonate (0.622 g, 4.50 mmol) and 1-chloroisoquinolin-6-ol (0.404 g, 2.25 mmol). The reaction mixture was heated to 80° C. for 8 h. After cooling to ambient temperature, the reaction mixture was diluted with ethyl acetate (20 mL) and washed with water (20 mL) and saturated sodium chloride (20 mL). The organic phase was dried over anhydrous sodium sulfate, filtered, and the filtrate was concentrated in vacuo.

The residue was purified by silica gel column chromatography, eluting with a gradient of 0 to 40% of ethyl acetate in heptane, to provide the title compound as a colorless oil (0.370 g, 65% yield): ¹H NMR (400 MHz, CDCl₃): δ 8.25 (d, J=9.3 Hz, 1H), 8.19 (d, J=5.7 Hz, 1H), 7.47 (d, J=5.7 Hz, 1H), 7.38 (dd, J=9.3, 2.5 Hz, 1H), 7.10 (d, J=2.5 Hz, 1H), 4.37 (d, J=20.6 Hz, 2H), 1.28 (dt, J=18.5, 7.3 Hz, 2H), 0.92-0.86 (m, 2H); MS (ES+) m/z 252.0 (M+1), 254.0 (M+1).

### Step 4. Preparation of N-(2-chloropyrimidin-5-yl)-6-((1-fluorocyclopropyl)methoxy)isoquinolin-1-amine

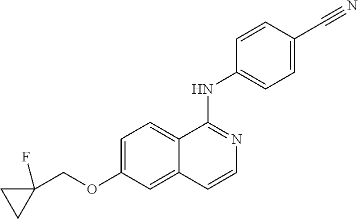 [SMILES: N#Cc1ccc(Nc2nccc3cc(OCC4(F)CC4)ccc23)cc1]

To a solution of 1-chloro-6-((1-fluorocyclopropyl)methoxy)isoquinoline (0.760 g, 3.01 mmol) in 1,4-dioxane (20 mL) was added 2-chloropyrimidin-5-amine (0.430 g, 3.32 mmol)), tris(dibenzylideneacetone)dipalladium(0) (0.276 g, 0.302 mmol), 2-dicyclohexylphosphino-2′,6′-dimethoxy-1,1′-biphenyl (0.274 g, 0.604 mmol) and potassium phosphate tribasic (0.961 g, 4.52 mmol). The reaction mixture was degassed by passing a stream of nitrogen through it for 5 minutes and was then heated to 120° C. in microwave reactor for 2 h. After cooling to ambient temperature, the reaction mixture was filtered through celite, and the filtrate was concentrated in vacuo. The obtained residue was purified by silica gel column chromatography, eluting with a gradient of 0 to 60% of ethyl acetate in heptane, to provide the title compound as a colourless solid (0.511 g, 48% yield): ¹H NMR (400 MHz, CDCl₃) δ 9.58 (s, 1H), 9.29 (s, 2H), 8.44 (d, J=9.3 Hz, 1H), 8.00 (d, J=5.8 Hz, 1H), 7.39 (dd, J=9.2, 2.5 Hz, 1H), 7.33 (d, J=2.4 Hz, 1H), 7.22 (d, J=5.8 Hz, 1H), 4.46 (d, J=22.7 Hz, 2H), 1.22-1.14 (m, 2H), 0.95-0.89 (m, 2H); MS (ES+) m/z 345.0 (M+1), 347.0 (M+1).

### Example 69

### Synthesis of 1-((6-chloropyridin-3-yl)amino)isoquinolin-6-ol

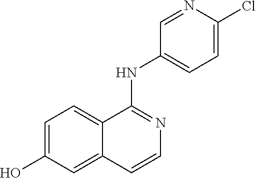 [SMILES: Oc1ccc2c(Nc3ccc(Cl)nc3)nccc2c1]

To a solution of 1-chloroisoquinolin-6-ol (0.400 g, 2.23 mmol) in 1,4-dioxane (20 mL), was added tris(dibenzylideneacetone)dipalladium(0) (0.204 g, 0.223 mmol), 2-dicyclohexylphosphino-2′,6′-dimethoxy-1,1′-biphenyl (0.183 g, 0.445 mmol), potassium phosphate tribasic (0.946 mg, 4.45 mmol) and 5-amino-2-chloropyridine (0.429 g, 3.34 mmol).

The mixture was degassed by passing a stream of nitrogen through it for 5 minutes. The reaction was heated to 120° C. in microwave reactor for 2 h. After cooling to ambient temperature, the reaction mixture was filtered through celite, and the filtrate was concentrated in vacuo. The residue was purified by silica gel column chromatography, eluting with a gradient of 0 to 40% of ethyl acetate in heptane, to provide the title compound as a colorless solid (0.330 g, 54% yield): ¹H NMR (400 MHz, DMSO-d₆) δ 10.29 (s, 1H), 9.31 (s, 1H), 8.87 (d, J=2.8 Hz, 1H), 8.42 (dd, J=8.7, 2.9 Hz, 1H), 8.38-8.36 (m, 1H), 7.89 (d, J=5.8 Hz, 1H), 7.69 (d, J=3.0 Hz, 1H), 7.15 (dd, J=9.1, 2.5 Hz, 1H), 7.05 (d, J=2.4 Hz, 1H), 6.98 (dd, J=8.5, 3.0 Hz, 1H); MS (ES+) m/z 272.4 (M+1), 274.4 (M+1).

### Example 70

### Synthesis of 6-(2-(1-oxa-6-azaspiro[3.3]heptan-6-yl)ethoxy)-N-(6-chloropyridin-3-yl)isoquinolin-1-amine

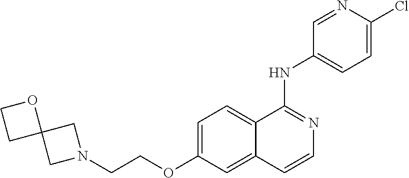 [SMILES: Clc1ccc(Nc2nccc3cc(OCCN4CC5(CCO5)C4)ccc23)cn1]

### Step 1. Preparation of 6-(2-bromoethoxy)-N-(6-chloropyridin-3-yl)isoquinolin-5-amine

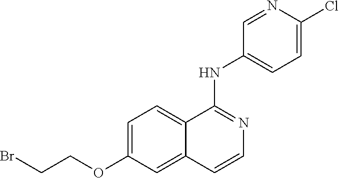 [SMILES: Clc1ccc(Nc2nccc3cc(OCCBr)ccc23)cn1]

To a solution of 1-((6-chloropyridin-3-yl)amino)isoquinolin-6-ol (0.095 g, 0.350 mmol) in N,N-dimethylformamide (5 mL) was added sodium hydride (60 wt. % dispersion in mineral oil, 0.042 g, 1.05 mmol) at 0° C. The reaction mixture was stirred allowed to warm to ambient temperature and stirred for 30 minutes. To the resulting reaction mixture was added 1,2-dibromoethane (0.120 mL, 1.40 mmol). The reaction mixture was stirred at 60° C. for 16 h. After cooling the ambient temperature, the reaction mixture was quenched by addition of water (20 mL). The mixture was diluted with ethyl acetate (20 mL) and washed with saturated aqueous ammonium chloride (20 mL) and brine (20 mL). The organic phase was dried over anhydrous sodium sulfate, filtered, and the filtrate was concentrated in vacuo. The obtained residue was purified by silica gel column chromatography, eluting with a gradient of 0 to 60% of ethyl acetate in heptane, to provide 6-(2-bromoethoxy)-N-(6-chloropyridin-3-yl)isoquinolin-1-amine as a colorless oil (0.045 g, 33% yield): MS (ES+) m/z 379.4 (M+1), 381.4 (M+1).

### Step 2. Preparation of 6-(2-(1-oxa-6-azaspiro[3.3]heptan-6-yl)ethoxy)-N-(6-chloropyridin-3-yl)isoquinolin-1-amine

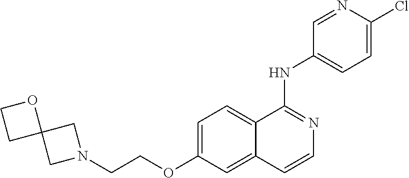 [SMILES: Clc1ccc(Nc2nccc3cc(OCCN4CC5(CCO5)C4)ccc23)cn1]

To a solution of 6-(2-bromoethoxy)-N-(6-chloropyridin-3-yl)isoquinolin-1-amine (0.035 g, 0.0928 mmol) in N,N-dimethylformamide (5 mL) was added 1-oxa-6-azaspiro[3.3]heptane hemioxalate (0.027 g, 0.0928 mmol) and potassium carbonate (0.026 g, 0.186 mmol). The reaction mixture was stirred at 80° C. for 16 h. After cooling to ambient temperature, the reaction mixture was diluted with ethyl acetate (20 mL) and washed with saturated ammonium chloride (20 mL). The organic phase was dried over anhydrous sodium sulfate, filtered, and the filtrate was concentrated in vacuo. The residue was purified by silica gel column chromatography, eluting with a gradient of 0 to 10% of methanol in dichloromethane, to provide the title compound as a colorless solid (0.0053 g, 13% yield): ¹H NMR (400 MHz, CDCl₃) δ 8.50 (d, J=2.8 Hz, 1H), 8.35 (dd, J=8.7, 2.8 Hz, 1H), 8.01 (d, J=5.7 Hz, 1H), 7.85 (d, J=9.2 Hz, 1H), 7.30 (d, J=8.7 Hz, 1H), 7.20 (dd, J=9.1, 2.5 Hz, 1H), 7.09 (t, J=5.5 Hz, 2H), 7.02 (d, J=2.4 Hz, 1H), 4.52 (t, J=7.6 Hz, 2H), 4.12 (t, J=5.3 Hz, 2H), 3.83-3.81 (m, 2H), 3.35 (dd, J=7.6, 2.2 Hz, 2H), 2.95 (t, J=5.3 Hz, 2H), 2.88 (t, J=7.6 Hz, 2H); MS (ES+) m/z 397.2 (M+1), 399.2 (M+1).

### Example 71

### Synthesis of 6-(2-(2-azaspiro[3.3]heptan-2-yl)ethoxy)-N-(6-chloropyridin-3-yl)isoquinolin-1-amine

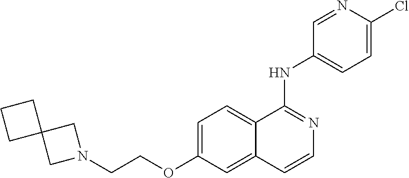 [SMILES: Clc1ccc(Nc2nccc3cc(OCCN4CC5(CCC5)C4)ccc23)cn1]

Following the procedure as described for EXAMPLE 70, Step 2, and making variations as required to replace 1-oxa-6-azaspiro[3.3]heptane hemioxalate with 2-azaspiro[3.3]heptane hemioxalate, the title compound was obtained as a colorless solid (0.0144 g, 18% yield): ¹H NMR (400 MHz, DMSO-d₆) δ 9.37 (s, 1H), 8.88 (d, J=2.8 Hz, 1H), 8.44-8.40 (m, 2H), 7.95 (d, J=5.8 Hz, 1H), 7.44 (d, J=8.7 Hz, 1H), 7.24 (t, J=3.1 Hz, 1H), 7.17 (d, J=5.8 Hz, 1H), 4.07 (t, J=5.5 Hz, 2H), 3.25 (s, 4H), 2.80 (t, J=5.1 Hz, 2H), 2.04 (t, J=7.6 Hz, 4H), 1.75 (quintet, J=7.5 Hz, 2H), NH not observed; MS (ES+) m/z 395.2 (M+1), 397.2 (M+1).

### Example 72

### Synthesis of N-(6-chloropyridin-3-yl)-6-(2-(3-methoxyazetidin-1-yl)ethoxy)isoquinolin-1-amine

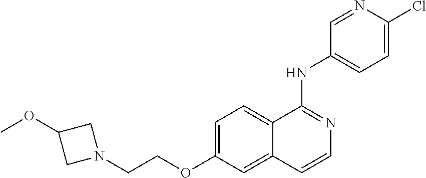 [SMILES: COC1CN(CCOc2ccc3c(Nc4ccc(Cl)nc4)nccc3c2)C1]

Following the procedure as described for EXAMPLE 70, Step 2, and making variations as required to replace 1-oxa-6-azaspiro[3.3]heptane hemioxalate with 3-methoxyazetidine hydrochloride, the title compound was obtained as a colorless solid (0.006 g, 10% yield): ¹H NMR (400 MHz, CD₃OD) δ 8.76-8.73 (m, 1H), 8.29-8.26 (m, 2H), 7.92 (d, J=5.9 Hz, 1H), 7.41 (dd, J=8.7, 0.5 Hz, 1H), 7.26-7.17 (m, 3H), 4.20 (t, J=5.2 Hz, 2H), 4.12 (quintet, J=5.8 Hz, 1H), 3.83-3.79 (m, 2H), 3.30 (s, 3H), 3.27-3.23 (m, 2H), 3.05 (t, J=5.2 Hz, 2H), NH not observed; MS (ES+) m/z 385.4 (M+1), 387.4 (M+1).

### Example 73

### Synthesis of 6-(2-(2-oxa-6-azaspiro[3.3]heptan-6-yl)ethoxy)-N-(6-chloropyridin-3-yl)isoquinolin-1-amine

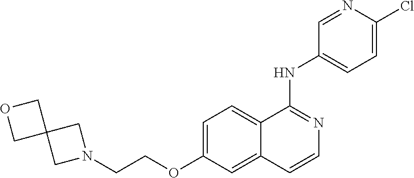 [SMILES: Clc1ccc(Nc2nccc3cc(OCCN4CC5(COC5)C4)ccc23)cn1]

Following the procedure as described for EXAMPLE 70, Step 2, and making variations as required to replace 1-oxa-6-azaspiro[3.3]heptane hemioxalate with 2-oxa-6-azaspiro[3.3]heptane hemioxalate, the title compound was obtained as a colorless solid (0.0041 g, 7.8% yield): ¹H NMR (400 MHz, CD₃OD) δ 8.74 (d, J=2.7 Hz, 1H), 8.28 (dd, J=8.8, 2.4 Hz, 2H), 7.92 (d, J=5.8 Hz, 1H), 7.41 (d, J=8.7 Hz, 1H), 7.25 (dd, J=9.2, 2.5 Hz, 1H), 7.20 (dd, J=9.3, 4.1 Hz, 2H), 4.77 (s, 4H), 4.17 (t, J=5.1 Hz, 2H), 3.59 (s, 4H), 2.93 (t, J=5.1 Hz, 2H), NH not observed; MS (ES+) m/z 397.2 (M+1), 399.2 (M+1).

### Example 74

### Synthesis of 6-(2-(1H-imidazol-1-yl)ethoxy)-N-(6-chloropyridin-3-yl)isoquinolin-1-amine

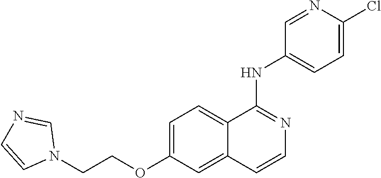 [SMILES: Clc1ccc(Nc2nccc3cc(OCCn4ccnc4)ccc23)cn1]

Following the procedure as described for EXAMPLE 70, Step 2, and making variations as required to replace 1-oxa-6-azaspiro[3.3]heptane hemioxalate with imidazole, the title compound was obtained as a colorless solid (0.0047 g, 9.7% yield): ¹H NMR (400 MHz, CD₃OD) δ 8.72 (d, J=2.7 Hz, 1H), 8.26 (td, J=5.8, 3.0 Hz, 2H), 7.99 (s, 1H), 7.90 (d, J=5.9 Hz, 1H), 7.41-7.36 (m, 2H), 7.25-7.21 (m, 2H), 7.16 (d, J=5.9 Hz, 1H), 7.09 (t, J=0.3 Hz, 1H), 4.56 (t, J=4.8 Hz, 2H), 4.46-4.44 (m, 2H), NH not observed; MS (ES+) m/z 366.2 (M+1), 368.2 (M+1).

### Example 75

### Synthesis of 2-(2-((1-((6-chloropyridin-3-yl)amino)isoquinolin-6-yl)oxy)ethyl)-1,2-dihydro-3H-pyrazol-3-one

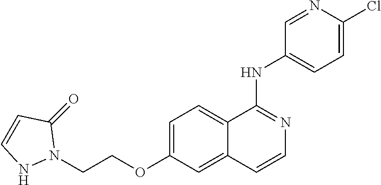 [SMILES: O=c1cc[nH]n1CCOc1ccc2c(Nc3ccc(Cl)nc3)nccc2c1]

Following the procedure as described for EXAMPLE 70, Step 2, and making variations as required to replace 1-oxa-6-azaspiro[3.3]heptane hemioxalate with 3H-pyrazol-3-one, the title compound was obtained as a colorless solid (0.0097 g, 19% yield): ¹H NMR (400 MHz, DMSO-d₆) δ 11.94 (s, 1H), 9.40 (s, 1H), 8.89 (d, J=2.8 Hz, 1H), 8.46-8.41 (m, 2H), 7.97 (d, J=5.7 Hz, 1H), 7.55 (dt, J=0.8, 0.4 Hz, 1H), 7.45 (d, J=8.8 Hz, 1H), 7.35-7.32 (m, 2H), 7.19 (d, J=5.8 Hz, 1H), 5.72 (dd, J=1.1, 0.6 Hz, 1H), 4.46 (s, 4H); MS (ES+) m/z 382.4 (M+1), 384.4 (M+1).

### Example 76

### Synthesis of N-(6-chloropyridin-3-yl)-6-fluoro-N-((2-(trimethylsilyl)ethoxy)-methyl)isoquinolin-1-amine

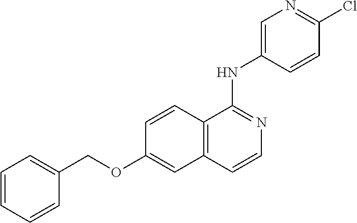 [SMILES: Clc1ccc(Nc2nccc3cc(OCc4ccccc4)ccc23)cn1]

### Step 1. Preparation of N-(6-chloropyridin-3-yl)-6-fluoroisoquinolin-1-amine

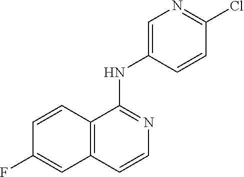 [SMILES: Fc1ccc2c(Nc3ccc(Cl)nc3)nccc2c1]

To a solution of 1-chloro-6-fluoroisoquinoline (3.0 g, 16.5 mmol) in 1,4-dioxane (150 mL) was added 5-amino-2-chloropyridine (3.18 g, 24.5 mmol), tris(dibenzylideneacetone)dipalladium(0) (1.51 g, 1.65 mmol), 2-dicyclohexylphosphino-2′,6′-dimethoxy-1,1′-biphenyl (1.35 g, 3.30 mmol), and potassium phosphate tribasic (7.01 g, 33.0 mmol). The reaction mixture was degassed by passing a stream of nitrogen through it for 10 minutes and then heated to 110° C. for 8 h. After cooling to ambient temperature, the reaction mixture was filtered. The filter cake was rinsed with ethyl acetate (50 mL) and the combined filtrate was concentrated in vacuo. The residue was purified by silica gel column chromatography, eluting with a gradient of 0 to 60% of ethyl acetate in heptane, to afford the title compound as a colorless solid (4.0 g, 88% yield): ¹H NMR (400 MHz, DMSO-d₆) δ9.54 (s, 1H), 8.88 (d, J=2.8 Hz, 1H), 8.64-8.61 (m, 1H), 8.41 (dd, J=8.7, 2.8 Hz, 1H), 8.05 (d, J=5.7 Hz, 1H), 7.69 (dd, J=9.9, 2.6 Hz, 1H), 7.62-7.56 (m, 1H), 7.48 (d, J=8.7 Hz, 1H), 7.28 (d, J=5.8 Hz, 1H); MS (ES+) m/z 274.5 (M+1), 276.5 (M+1).

### Step 2. Preparation of N-(6-chloropyridin-3-yl)-6-fluoro-N-((2-(trimethylsilyl)ethoxy)methyl)isoquinolin-1-amine

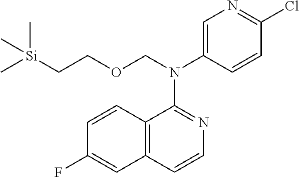 [SMILES: C[Si](C)(C)CCOCN(c1ccc(Cl)nc1)c1nccc2cc(F)ccc12]

To a solution of N-(6-chloropyridin-3-yl)-6-fluoroisoquinolin-1-amine (4.0 g, 12.4 mmol) in N,N-dimethylformamide (40 mL) was added sodium hydride (60% dispersion in mineral oil, 0.99 g, 24.8 mmol) at 0° C. The reaction mixture was allowed to warm to ambient temperature and stirred for 20 minutes. To the reaction mixture was then added 2-(trimethylsilyl)ethoxymethyl chloride (3.10 g, 18.6 mmol). The reaction mixture was stirred at ambient temperature for 16 h.

The reaction mixture was diluted with saturated ammonium chloride solution (50 mL) and extracted with ethyl acetate (2×50 mL). The combined organic phase was washed with brine (50 mL), dried over anhydrous sodium sulfate, and filtered. The filtrate was concentrated in vacuo to provide a residue which was purified by silica gel chromatography, eluting with a gradient of 0 to 30% of ethyl acetate in heptane, to afford the title compound as a colorless oil (3.8 g, 75% yield): ¹H NMR (400 MHz, CDCl₃) δ 8.36 (d, J=5.7 Hz, 1H), 8.02 (d, J=2.8 Hz, 1H), 7.81 (dd, J=9.3, 5.4 Hz, 1H), 7.51 (d, J=5.7 Hz, 1H), 7.46 (dd, J=9.0, 2.5 Hz, 1H), 7.20-7.11 (m, 3H), 5.41 (s, 2H), 3.54 (t, J=8.2 Hz, 2H), 0.87 (t, J=8.2 Hz, 2H), −0.12 (s, 9H); MS (ES+) m/z 404.4 (M+1), 406.4 (M+1).

### Step 3. Preparation of 6-(benzyloxy)-N-(6-chloropyridin-3-yl)isoquinolin-1-amine

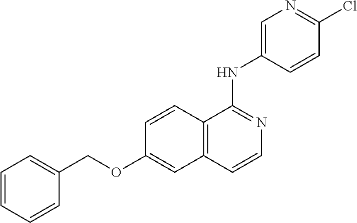 [SMILES: Clc1ccc(Nc2nccc3cc(OCc4ccccc4)ccc23)cn1]

To a solution of phenylmethanol (0.0482 g, 0.445 mmol) in N,N-dimethylformamide (6 mL) was added sodium hydride (60% dispersion in mineral oil, 0.0178 g, 0.445 mmol) at 0° C. and the resulting mixture was stirred for 20 minutes at this temperature. To the reaction mixture was then added N-(6-chloropyridin-3-yl)-6-fluoro-N-((2-(trimethylsilyl)ethoxy)methyl)isoquinolin-1-amine (0.063 g, 0.148 mmol) at 0° C. The reaction mixture was allowed to warm to ambient temperature and then heated to 60° C. for 16 h. The mixture was quenched by addition of water (20 mL). After dilution with ethyl acetate (20 mL), the mixture was washed with saturated aqueous ammonium chloride (20 mL) and brine (20 mL). The organic phase was dried over anhydrous sodium sulfate, filtered, and the filtrate was concentrated in vacuo. The obtained residue was dissolved in dichloromethane (3 mL). To this mixture was added trifluoroacetic acid (1.13 mL, 14.8 mmol) at ambient temperature. The reaction mixture was stirred at ambient temperature for 1 h. To the mixture was added saturated sodium bicarbonate solution (20 mL), and the mixture was extracted with ethyl acetate (2×20 mL). The combined organic phase was washed with brine (20 mL), dried over anhydrous sodium sulfate, and filtered. The filtrate was concentrated in vacuo to provide a residue which was purified by silica gel chromatography, eluting with a gradient of 10 to 100% of ethyl acetate in heptane, afford the title compound as a colorless solid (0.031 g, 58% yield): ¹H NMR (400 MHz, CDCl₃) δ 7.80 (d, J=2.7 Hz, 1H), 7.66 (d, J=10.3 Hz, 1H), 7.22 (dd, J=8.5, 2.8 Hz, 1H), 6.85 (dd, J=8.6, 0.2 Hz, 1H), 6.74-6.70 (i, 5H), 6.63-6.51 (m, 4H), 4.53 (, 2H), NH not observed; MS (ES+) m/z 362.2 (M+1), 364.2 (M+1).

### Examples 77-110

In a similar manner as described in EXAMPLE 76, Step 3, utilizing the appropriately substituted starting materials and intermediates, the following compounds were prepared:

| | | MS | |
|---|---|---|---|
| | | (ES+) | |
| Example No | Name | m/z | NMR |
| 77 | 1-(((1-((6-chloropyridin- | 365.2 | ¹H NMR (400 MHz, DMSO-d₆) δ 9.43 |
| | 3-yl)amino)isoquinolin- | (M + 1), | (s, 1H), 8.90 (d, J = 2.7 Hz, 1H), 8.49 |
| | 6-yl)oxy)methyl)cyclo- | 367.2 | (d, J = 8.9 Hz, 1H), 8.44 (dd, J = 8.8, |
| | butane-1-carbonitrile | (M + 1). | 2.9 Hz, 1H), 7.99 (d, J = 5.7 Hz, 1H), |
| | | | 7.46 (d, J = 8.7 Hz, 1H), 7.38-7.35 (m, |
| | | | 2H), 7.20 (d, J = 5.9 Hz, 1H), 4.45 (s, |
| | | | 2H), 2.57-2.54 (m, 2H), 2.34-2.28 (m, |
| | | | 2H), 2.20-2.08 (m, 2H). |
| 78 | 3-((1-((6-chloropyridin- | 353.2 | ¹H NMR (400 MHz, DMSO-d₆) δ 9.43 |
| | 3-yl)amino)isoquinolin- | (M + 1), | (s, 1H), 8.89 (d, J = 2.8 Hz, 1H), 8.49 |
| | 6-yl)oxy)-2,2- | 355.0 | (d, J = 8.9 Hz, 1H), 8.44 (dd, J = 8.8, |
| | dimethylpropanenitrile | (M + 1). | 2.9 Hz, 1H), 7.99 (d, J = 5.8 Hz, 1H), |
| | | | 7.46 (d, J = 8.8 Hz, 1H), 7.38-7.36 (m, |
| | | | 2H), 7.20 (d, J = 5.8 Hz, 1H), 4.20 (s, |
| | | | 2H), 1.48 (s, 6H). |
| 79 | rac-(3R,4S)-4-((1-((6- | 358.0 | ¹H NMR (400 MHz, DMSO-d₆) δ 9.39 |
| | chloropyridin-3- | (M + 1), | (s, 1H), 8.89 (d, J = 2.8 Hz, 1H), 8.43 |
| | yl)amino)isoquinolin-6- | 360.0 | (dd, J = 8.8, 2.9 Hz, 2H), 7.96 (d, J = |
| | yl)oxy)tetrahydrofuran- | (M + 1). | 5.8 Hz, 1H), 7.45 (d, J = 8.7 Hz, 1H), |
| | 3-ol | | 7.36-7.31 (m, 2H), 7.18 (d, J = 5.8 Hz, |
| | | | 1H), 5.18 (d, J = 6.0 Hz, 1H), 4.99- |
| | | | 4.96 (m, 1H), 4.50 (quintet, J = 5.5 |
| | | | Hz, 1H), 4.14 (dd, J = 9.5, 5.6 Hz, |
| | | | 1H), 3.95 (dd, J = 8.7, 5.8 Hz, 1H), |
| | | | 3.80 (dd, J = 9.5, 4.5 Hz, 1H), 3.63 |
| | | | (dd, J = 8.7, 5.6 Hz, 1H). |
| 80 | 1-(((1-((6-chloropyridin- | 351.2 | ¹H NMR (400 MHz, DMSO-d₆): δ |
| | 3-yl)amino)isoquinolin- | (M + 1), | 9.42 (s, 1H), 8.89 (d, J = 2.8 Hz, 1H), |
| | 6-yl)oxy)methyl)cyclo- | 353.2 | 8.48 (d, J = 9.2 Hz, 1H), 8.43 (dd, J = |
| | propane-1-carbonitrile | (M + 1). | 8.8, 2.8 Hz, 1H), 7.98 (d, J = 5.8 Hz, |
| | | | 1H), 7.46 (d, J = 8.7 Hz, 1H), 7.37 |
| | | | (dd, J = 9.2, 2.6 Hz, 1H), 7.27 (d, J = |
| | | | 2.6 Hz, 1H), 7.17 (d, J = 5.8 Hz, 1H), |
| | | | 4.22 (s, 2H), 1.43 (m, 2H), 1.23 (m, 2H). |
| 81 | 6-((2- | 382.0 | ¹H NMR (400 MHz, DMSO-d₆) δ 9.38 |
| | oxaspiro[3.3]heptan-6- | (M + 1), | (s, 1H), 8.88 (d, J = 2.8 Hz, 1H), 8.44- |
| | yl)methoxy)-N-(6- | 384.0 | 8.41 (m, 2H), 7.96 (d, J = 5.8 Hz, 1H), |
| | chloropyridin-3- | (M + 1). | 7.45 (d, J = 8.7 Hz, 1H), 7.26 (dd, J = |
| | yl)isoquinolin-1-amine | | 7.1, 2.5 Hz, 2H), 7.17 (d, J = 5.8 Hz, |
| | | | 1H), 4.60 (s, 2H), 4.56 (s, 2H), 4.05 |
| | | | (d, J = 6.3 Hz, 2H), 2.59-2.55 (m, 1H), |
| | | | 2.39 (t, J = 6.4 Hz, 2H), 2.12-2.07 (m, 2H). |
| 82 | 6-((1H-pyrazol-1- | 352.0 | ¹H NMR (400 MHz, DMSO-d₆) δ 8.83 |
| | yl)methoxy)-N-(6- | (M + 1), | (dd, J = 2.3, 0.3 Hz, 1H), 8.51 (d, J = |
| | chloropyridin-3- | 354.0 | 9.3 Hz, 1H), 8.36-8.33 (m, 1H), 8.12 |
| | yl)isoquinolin-1-amine | (M + 1). | (d, J = 2.3 Hz, 1H), 7.92-7.90 (m, 1H), |
| | | | 7.64-7.62 (m, 2H), 7.55-7.52 (m, 1H), |
| | | | 7.46-7.43 (m, 1H), 7.23 (d, J = 6.0 Hz, |
| | | | 1H), 6.38 (t, J = 2.1 Hz, 1H), 6.29 (s, |
| | | | 2H), NH not observed. |
| 83 | N-(6-chloropyridin-3- | 390.2 | ¹H NMR (400 MHz, DMSO-d₆) δ 9.40 |
| | yl)-6-((3,3-difluorocyclo- | (M + 1), | (s, 1H), 8.88 (d, J = 2.8 Hz, 1H), 8.43 |
| | hexyl)oxy)isoquinolin- | 392.2 | (dt, J = 9.0, 4.6 Hz, 2H), 7.96 (d, J = |
| | 1-amine | (M + 1). | 5.8 Hz, 1H), 7.46 (s, 1H), 7.36 (d, J = |
| | | | 2.6 Hz, 1H), 7.27 (dd, J = 9.2, 2.6 Hz, |
| | | | 1H), 7.20 (d, J = 5.9 Hz, 1H), 4.80- |
| | | | 4.75 (m, 1H), 2.12-1.85 (m, 6H), 1.67- |
| | | | 1.54 (m, 2H). |
| 84 | N-(6-chloropyridin-3- | 408.0 | ¹H NMR (400 MHz, CD₃CN) δ 8.81 (d, |
| | yl)-6-((6,7-dihydro-5H- | (M + 1), | J = 0.2 Hz, 1H), 8.37-8.34 (m, 1H), |
| | pyrazolo[5,1- | 410.0 | 8.17-8.14 (m, 1H), 7.99-7.98 (m, 2H), |
| | b][1,3]oxazin-3- | (M + 1). | 7.40 (s, 1H), 7.36-7.33 (m, 1H), 7.24- |
| | yl)methoxy)isoquinolin- | | 7.18 (m, 2H), 4.96 (s, 2H), 4.38-4.37 |
| | 1-amine | | (m, 2H), 4.13-4.10 (m, 2H), 1.81-1.78 |
| | | | (m, 2H), NH not observed. |
| 85 | 3-(((1-((6-chloropyridin- | 367.0 | ¹H NMR (400 MHz, DMSO-d₆) δ 9.44 |
| | 3-yl)amino)isoquinolin- | (M + 1), | (s, 1H), 8.90 (d, J = 2.8 Hz, 1H), 8.50 |
| | 6-yl)oxy)methyl)oxetane- | 369.0 | (d, J = 9.3 Hz, 1H), 8.44 (dd, J = 8.8, |
| | 3-carbonitrile | (M + 1). | 2.9 Hz, 1H), 8.00 (d, J = 5.8 Hz, 1H), |
| | | | 7.46 (d, J = 8.7 Hz, 1H), 7.41-7.35 (m, |
| | | | 2H), 7.21 (d, J = 5.8 Hz, 1H), 4.94 (d, |
| | | | J = 6.6 Hz, 2H), 4.71 (d, J = 6.4 Hz, 4H). |
| 86 | N-(6-chloropyridin-3- | 378.0 | ¹H NMR (400 MHz, DMSO-d₆) δ 9.96- |
| | yl)-6-((4,4- | (M + 1), | 9.84 (m, 1H), 8.83-8.82 (m, 1H), 8.55 |
| | difluorotetrahydrofuran- | 380.0 | (d, J = 10.0 Hz, 1H), 8.35-8.32 (m, |
| | 3-yl)oxy)isoquinolin-1- | (M + 1). | 1H), 7.90-7.89 (m, 1H), 7.55 (dd, J = |
| | amine 2,2,2- | | 8.7, 0.4 Hz, 1H), 7.46-7.44 (m, 2H), |
| | trifluoroacetate | | 7.26-7.24 (m, 1H), 5.44-5.38 (m, 1H), |
| | | | 4.49-4.45 (m, 1H), 4.21-3.98 (m, 3H); |
| | | | ¹⁹F NMR (376 MHz, DMSO-d₆) δ −74.09 |
| | | | (s, 4F), -103.92 (d, J = 239.5 |
| | | | Hz, 1F), -119.71 (d, J = 239.6 Hz, 1F). |
| 87 | N-(6-chloropyridin-3- | 367.2 | ¹H NMR (400 MHz, DMSO-d₆) δ 9.40 |
| | yl)-6-((4-methyl-4H- | (M + 1), | (s, 1H), 8.88 (d, J = 2.8 Hz, 1H), 8.54 |
| | 1,2,4-triazol-3- | 369.2 (M | (s, 1H), 8.47 (d, J = 9.2 Hz, 1H), 8.42 |
| | yl)methoxy)isoquinolin- | + 1). | (dd, J = 8.8, 2.8 Hz, 1H), 7.99 (d, J = |
| | 1-amine | | 5.6 Hz, 1H), 7.51 (d, J = 2.8 Hz, 1H), |
| | | | 7.45 (d, J = 8.8 Hz, 1H), 7.37 (dd, J = |
| | | | 9.2, 2.8 Hz, 1H), 7.19 (d, J = 5.6 Hz, |
| | | | 1H), 5.48 (s, 2H), 3.74 (s, 3H). |
| 88 | 1-(((1-((2- | 381.0 | ¹H NMR (400 MHz, DMSO-d₆) δ 9.95- |
| | methylpyrimidin-5- | (M + 1), | 9.87 (m, 1H), 8.83 (d, J = 2.7 Hz, 1H), |
| | yl)amino)isoquinolin-6- | 383.0 | 8.55-8.53 (m, 1H), 8.35-8.33 (m, 1H), |
| | yl)oxy)methyl)cyclopro- | (M + 1). | 7.89 (d, J = 6.0 Hz, 1H), 7.54 (d, J = |
| | pane-1-carbonitrile | | 8.7 Hz, 1H), 7.44 (d, J = 8.4 Hz, 2H), |
| | | | 7.23 (d, J = 6.2 Hz, 1H), 4.48 (d, J = |
| | | | 10.4 Hz, 1H), 4.40 (d, J = 10.3 Hz, |
| | | | 1H), 4.00 (td, J = 6.7, 1.9 Hz, 2H), |
| | | | 2.46 (m, 1H), 2.26 (m, 1H), 2.10 (m, 2H). |
| 89 | N-(6-chloropyridin-3- | 356.2 | ¹H NMR (400 MHz, DMSO-d₆) δ |
| | yl)-6-(2-(oxetan-3- | (M + 1), | 10.18 (s, 1H), 8.78 (d, J = 2.6 Hz, |
| | yl)ethoxy)isoquinolin-1- | 358.2 | 1H), 8.52 (d, J = 9.2 Hz, 1H), 8.28- |
| | amine | (M + 1). | 8.25 (m, 1H), 7.80 (d, J = 6.2 Hz, 1H), |
| | | | 7.59 (d, J = 8.6 Hz, 1H), 7.39-7.34 (m, |
| | | | 2H), 7.26 (d, J = 6.3 Hz, 1H), 4.70 |
| | | | (dd, J = 7.9, 5.9 Hz, 2H), 4.40 (t, J= |
| | | | 6.2 Hz, 2H), 4.19-4.11 (m, 2H), 3.22- |
| | | | 3.15 (m, 1H), 2.17 (q, J = 6.9 Hz, 2H). |
| 90 | 6-((1-benzylpiperidin-4- | 445.2 | ¹H NMR (400 MHz, DMSO-d₆) δ |
| | yl)oxy)-N-(6- | (M + 1), | 10.09-9.51 (m, 2H), 8.83-8.81 (m, |
| | chloropyridin-3- | 447.2 | 1H), 8.56-8.30 (m, 2H), 7.89-7.86 (m, |
| | yl)isoquinolin-1-amine | (M + 1). | 1H), 7.60-7.33 (m, 8H), 7.21-7.17 (m, |
| | trifluoroacetate | | 1H), 4.43-4.27 (m, 3H), 3.34-3.10 (m, |
| | | | 4H), 2.23-2.05 (m, 2H), 1.99-1.72 (m, 2H). |
| 91 | N-(6-chloropyridin-3- | 358.2 | ¹H NMR (400 MHz, DMSO-d₆) δ |
| | yl)-6-(1-(1- | (M + 1), | 10.09 (s, 1H), 8.79 (d, J = 2.6 Hz, |
| | fluorocyclopropyl)ethoxy)isoquinolin- | 360.2 | 1H), 8.53 (d, J = 9.1 Hz, 1H), 8.30- |
| | 1-amine | (M + 1). | 8.27 (m, 1H), 7.82-7.81 (m, 1H), 7.58 |
| | | | (d, J = 8.6 Hz, 1H), 7.47-7.41 (m, 2H), |
| | | | 7.22 (d, J = 6.2 Hz, 1H), 4.88-4.77 (m, |
| | | | 1H), 1.44 (d, J = 6.3 Hz, 3H), 1.15- |
| | | | 1.05 (m, 2H), 0.93-0.87 (m, 2H). |
| 92 | N-(6-chloropyridin-3- | 354.2 | ¹H NMR (400 MHz, DMSO-d₆) δ |
| | yl)-6-fluoro-N-((2- | (M + 1), | 10.14 (s, 1H), 8.78-8.77 (m, 1H), 8.50 |
| | (trimethylsilyl)ethoxy)- | 356.2 | (d, J = 9.1 Hz, 1H), 8.26 (d, J = 9.4 |
| | methyl)isoquinolin-1- | (M + 1). | Hz, 1H), 7.78 (d, J = 6.2 Hz, 1H), 7.59 |
| | amine | | (d, J = 8.4 Hz, 1H), 7.43 (s, 1H), 7.37 |
| | | | (d, J = 9.9 Hz, 1H), 7.26 (d, J = 6.3 |
| | | | Hz, 1H), 4.64-4.60 (m, 1H), 2.05-2.01 |
| | | | (m, 2H), 1.79-1.73 (m, 2H), 1.61-1.23 |
| | | | (m, 6H). |
| 93 | N-(6-chloropyridin-3- | 390.2 | ¹H NMR (400 MHz, CD₃OD) δ 8.74 (d, |
| | yl)-6-((4,4- | (M + 1), | J = 2.7 Hz, 1H), 8.28 (td, J = 5.7, 3.1 |
| | difluorocyclohexyl)oxy)isoquinolin- | 392.2 | Hz, 2H), 7.92 (d, J = 5.9 Hz, 1H), 7.41 |
| | 1-amine | (M + 1). | (dd, J = 8.7, 0.4 Hz, 1H), 7.29-7.25 |
| | | | (m, 2H), 7.19-7.17 (m, 1H), 4.84-4.81 |
| | | | (m, 1H), 2.24-2.10 (m, 2H), 2.09-1.99 |
| | | | (m, 6H), NH not observed. |
| 94 | N-(6-chloropyridin-3- | 364.2 | ¹H NMR (400 MHz, CD₃OD) δ 8.88 (s, |
| | yl)-6-(pyrazin-2- | (M + 1), | 1H), 8.73 (d, J = 2.8 Hz, 1H), 8.67 |
| | ylmethoxy)isoquinolin- | 366.2 | (dd, J = 2.3, 1.6 Hz, 1H), 8.61 (d, J = |
| | 1-amine | (M + 1). | 2.6 Hz, 1H), 8.32-8.26 (m, 2H), 7.92 |
| | | | (d, J = 5.9 Hz, 1H), 7.41-7.35 (m, 3H), |
| | | | 7.18 (d, J = 5.8 Hz, 1H), 5.43 (s, 2H), |
| | | | NH not observed. |
| 95 | rac-(1R,3S)-3-((1-((6- | 370.2 | ¹H NMR (400 MHz, CD₃OD) δ 8.78- |
| | chloropyridin-3- | (M + 1), | 8.62 (m, 1H), 8.27 (d, J = 8.8 Hz, 1H), |
| | yl)amino)isoquinolin-6- | 372.2 | 8.24-8.21 (m, 1H), 8.17 (t, J = 0.5 Hz, |
| | yl)oxy)cyclohexan-1-ol | (M + 1). | 1H), 7.85 (s, 1H), 7.51-7.37 (m, 1H), |
| | | | 7.22 (d, J = 2.0 Hz, 2H), 7.18 (d, J = |
| | | | 6.0 Hz, 1H), 4.57-4.50 (m, 1H), 3.78- |
| | | | 3.71 (m, 1H), 2.51-2.48 (m, 1H), 2.21- |
| | | | 2.18 (m, 1H), 2.02-1.99 (m, 1H), 1.92- |
| | | | 1.87 (m, 1H), 1.49-1.25 (m, 4H), NH |
| | | | not observed. |
| 96 | (1r,3r)-3-((1-((6- | 342.2 | ¹H NMR (400 MHz, CD₃OD) δ 8.74 (s, |
| | chloropyridin-3- | (M + 1), | 1H), 8.28-8.25 (m, 2H), 7.92-7.90 (m, |
| | yl)amino)isoquinolin-6- | 344.2 | 1H), 7.41 (d, J = 8.7 Hz, 1H), 7.21- |
| | yl)oxy)cyclobutan-1-ol | (M + 1). | 7.16 (m, 2H), 7.01 (s, 1H), 5.06-5.01 |
| | | | (m, 1H), 4.59-4.53 (m, 1H), 2.57-2.46 |
| | | | (m, 4H), NH and OH not observed. |
| 97 | 6-((8-benzyl-8- | 471.2 | ¹H NMR (400 MHz, DMSO-d₆) δ 9.39 |
| | azabicyclo[3.2.1]octan- | (M + 1), | (s, 1H), 8.87 (d, J = 2.7 Hz, 1H), 8.46- |
| | 3-yl)oxy)-N-(6- | 473.2 | 8.41 (m, 2H), 8.26 (s, 1H), 7.94 (d, |
| | chloropyridin-3- | (M + 1). | J = 5.8 Hz, 1H), 7.44 (d, J = 8.7 Hz, |
| | yl)isoquinolin-1-amine | | 1H), 7.36-7.30 (m, 5H), 7.25-7.22 (m, |
| | | | 1H), 7.19 (d, J = 5.8 Hz, 1H), 4.65 (t, |
| | | | J = 4.5 Hz, 1H), 2.59 (d, J = 10.2 Hz, |
| | | | 2H), 2.44-2.36 (m, 6H), 1.82-1.76 (m, 4H). |
| 98 | N-(6-chloropyridin-3- | 370.2 | ¹H NMR (400 MHz, DMSO-d₆) δ 9.39 |
| | yl)-6-(2-(3- | (M + 1), | (s, 1H), 8.88 (d, J = 2.6 Hz, 1H), 8.43 |
| | methyloxetan-3- | 372.2 | (dt, J = 8.9, 2.5 Hz, 2H), 7.96 (d, J = |
| | yl)ethoxy)isoquinolin-1- | (M + 1). | 5.8 Hz, 1H), 7.45 (d, J = 8.7 Hz, 1H), |
| | amine | | 7.31 (d, J = 2.5 Hz, 1H), 7.24 (dd, J = |
| | | | 9.2, 2.6 Hz, 1H), 7.18 (d, J = 5.8 Hz, |
| | | | 1H), 4.50 (d, J = 5.7 Hz, 2H), 4.27 (d, |
| | | | J = 5.7 Hz, 2H), 4.20 (t, J = 6.7 Hz, |
| | | | 2H), 2.15 (t, J = 6.6 Hz, 2H), 1.36 (s, 3H). |
| 99 | N-(6-chloropyridin-3- | 380.2 | ¹H NMR (400 MHz, CDCl₃) δ 8.51 (d, |
| | yl)-6-(2-(1-methyl-1H- | (M + 1), | J = 2.8 Hz, 1H), 8.37 (dd, J = 8.7, 2.8 |
| | pyrazol-4- | 382.0 | Hz, 1H), 8.03 (d, J = 5.8 Hz, 1H), 7.89 |
| | yl)ethoxy)isoquinolin-1- | (M + 1). | (d, J = 9.2 Hz, 1H), 7.47 (d, J = 6.9 |
| | amine | | Hz, 1H), 7.33 (s, 1H), 7.29 (d, J = 9.1 |
| | | | Hz, 1H), 7.22 (dd, J = 9.1, 2.4 Hz, |
| | | | 1H), 7.11 (d, J = 5.8 Hz, 1H), 7.06 (d, |
| | | | J = 2.4 Hz, 1H), 4.24 (t, J = 6.7 Hz, |
| | | | 2H), 3.90 (s, 3H), 3.04 (t, J = 6.6 Hz, |
| | | | 2H), (NH not observed). |
| 100 | Cis-4-((1-((6- | 370.2 | ¹H NMR (400 MHz, DMSO-d₆) δ 9.61 |
| | chloropyridin-3- | (M + 1), | (br s, 1H), 8.84-8.84 (m, 1H), 8.46 |
| | yl)amino)isoquinolin-6- | 372.2 | (dd, J = 9.1, 0.3 Hz, 1H), 8.38-8.35 |
| | yl)oxy)cyclohexan-1-ol | (M + 1). | (m, 1H), 7.89-7.87 (m, 1H), 7.52-7.49 |
| | | | (m, 1H), 7.35-7.30 (m, 2H), 7.21-7.19 |
| | | | (m, 1H), 4.67-4.65 (m, 1H), 4.56 (br s, |
| | | | 1H), 3.69-3.64 (m, 1H), 1.95-1.87 (m, |
| | | | 2H), 1.75-1.69 (m, 2H), 1.65-1.61 (m, 4H). |
| 101 | N-(6-chloropyridin-3- | 413.2 | ¹H NMR (400 MHz, DMSO-d₆) δ 9.39 |
| | yl)-6-(2-methyl-2- | (M + 1), | (s, 1H), 8.89 (d, J = 2.8 Hz, 1H), 8.46- |
| | morpholinopropoxy)isoquinolin- | 415.2 | 8.42 (m, 2H), 7.97 (d, J = 5.7 Hz, 1H), |
| | 1-amine | (M + 1). | 7.45 (d, J = 8.7 Hz, 1H), 7.34-7.30 (m, |
| | | | 2H), 7.19 (d, J = 5.9 Hz, 1H), 4.02- |
| | | | 4.01 (m, 2H), 3.58-3.56 (m, 4H), 2.68- |
| | | | 2.62 (m, 4H), 1.17 (d, J = 0.3 Hz, 6H). |
| 102 | rac-(1S,3R)-3-((1-((6- | 356.2 (M | ¹H NMR (400 MHz, DMSO-d₆) δ 9.39 |
| | chloropyridin-3- | + 1), | (s, 1H), 8.89 (d, J = 2.8 Hz, 1H), 8.43 |
| | yl)amino)isoquinolin-6- | 358.2 (M | (dd, J = 8.8, 2.8 Hz, 2H), 7.95 (d, J = |
| | yl)oxy)cyclopentan-1-ol | + 1). | 5.8 Hz, 1H), 7.45 (d, J = 8.7 Hz, 1H), |
| | | | 7.23 (dd, J = 9.1, 2.5 Hz, 1H), 7.21 (d, |
| | | | J = 2.5 Hz, 1H), 7.18 (d, J = 5.8 Hz, |
| | | | 1H), 4.93-4.87 (m, 1H), 4.71 (d, J = |
| | | | 4.0 Hz, 1H), 4.19-4.12 (m, 1H), 2.48- |
| | | | 2.43 (m, 1H), 2.12-2.03 (m, 1H), 1.94- |
| | | | 1.86 (m, 1H), 1.82-1.74 (m, 1H), 1.72- |
| | | | 1.65 (m, 1H), 1.64-1.58 (m, 1H). |
| 103 | rac-(1R,3R)-3-((1-((6- | 356.2 | ¹H NMR (400 MHz, DMSO-d₆) |
| | chloropyridin-3- | (M + 1), | δ 8.94-8.75 (m, 1H), 8.54-8.23 (m, |
| | yl)amino)isoquinolin-6- | 358.2 | 2H), 7.94-7.84 (m, 1H), 7.57-7.21 (m, |
| | yl)oxy)cyclopentan-1-ol | (M + 1). | 4H), 5.13-5.05 (m, 1H), 4.33-4.30 (m, |
| | | | 1H), 2.53 (d, J = 1.8 Hz, 1H), 2.32- |
| | | | 2.24 (m, 1H), 2.13-2.06 (m, 1H), 1.98- |
| | | | 1.89 (m, 1H), 1.77-1.69 (m, 1H), 1.63- |
| | | | 1.56 (m, 1H), OH and NH not |
| | | | observed. |
| 104 | N-(6-chloropyridin-3- | 384.0 | ¹H NMR (400 MHz, DMSO-d₆) δ 9.36 |
| | yl)-6-(((1s,4s)-4- | (M + 1), | (s, 1H), 8.88 (d, J = 2.7 Hz, 1H), 8.43 |
| | methoxycyclohexyl)oxy)isoquinolin- | 386.0 | (dd, J = 8.8, 2.5 Hz, 2H), 7.95 (d, J = |
| | 1-amine | (M + 1). | 5.8 Hz, 1H), 7.45 (d, J = 8.7 Hz, 1H), |
| | | | 7.33 (d, J = 2.5 Hz, 1H), 7.25 (dd, J = |
| | | | 9.2, 2.4 Hz, 1H), 7.18 (d, J = 5.8 Hz, |
| | | | 1H), 4.64-4.57 (m, 1H), 3.31-3.25 (m, |
| | | | 4H), 2.11-1.97 (m, 4H), 1.56-1.38 (m, |
| | | | 4H). |
| 105 | N-(6-chloropyridin-3- | 384.0 | ¹H NMR (400 MHz, DMSO-d₆) δ 9.37 |
| | yl)-6-(((1r,4r)-4- | (M + 1), | (s, 1H), 8.88 (d, J = 2.8 Hz, 1H), 8.43 |
| | methoxycyclohexyl)oxy)isoquinolin- | 386.0 | (dd, J = 8.8, 2.8 Hz, 2H), 7.95 (d, J = |
| | 1-amine | (M + 1). | 5.8 Hz, 1H), 7.45 (d, J = 8.7 Hz, 1H), |
| | | | 7.32 (d, J = 2.5 Hz, 1H), 7.27 (dd, J = |
| | | | 9.2, 2.5 Hz, 1H), 7.17 (d, J = 5.8 Hz, |
| | | | 1H), 4.69-4.63 (m, 1H), 3.30-3.24 (m, |
| | | | 4H), 1.84-1.66 (m, 8H). |
| 106 | N-(6-chloropyridin-3- | 391.2 | ¹H NMR (400 MHz, DMSO-d₆) δ 9.37 |
| | yl)-6-((1-(pyridin-3- | (M + 1), | (s, 1H), 8.87 (d, J = 2.8 Hz, 1H), 8.54 |
| | yl)propan-2- | 393.2 | (d, J = 1.9 Hz, 1H), 8.48-8.40 (m, 3H), |
| | yl)oxy)isoquinolin-1- | (M + 1). | 7.95 (d, J = 5.8 Hz, 1H), 7.77-7.74 (m, |
| | amine | | 1H), 7.45 (d, J = 8.8 Hz, 1H), 7.35- |
| | | | 7.32 (m, 2H), 7.23 (dd, J = 9.2, 2.5 |
| | | | Hz, 1H), 7.17 (d, J = 5.7 Hz, 1H), |
| | | | 5.01-4.93 (m, 1H), 3.05-3.03 (m, 2H), |
| | | | 1.35-1.28 (m, 3H). |
| 107 | 3-(2-((1-((6- | 385.2 | ¹H NMR (400 MHz, DMSO-d₆) δ 9.40 |
| | chloropyridin-3- | (M + 1), | (d, J = 2.4 Hz, 1H), 8.88 (d, J = 2.8 |
| | yl)amino)isoquinolin-6- | 387.2 | Hz, 1H), 8.47-8.41 (m, 2H), 7.97 (d, |
| | yl)oxy)ethyl)oxazolidin- | (M + 1). | J = 5.7 Hz, 1H), 7.45 (d, J = 8.7 Hz, |
| | 2-one | | 1H), 7.34-7.28 (m, 2H), 7.19 (d, J = |
| | | | 5.8 Hz, 1H), 4.31-4.26 (m, 4H), 3.72- |
| | | | 3.68 (m, 2H), 3.62 (t, J = 5.2 Hz, 2H). |
| 108 | (R)-6-((1- | 445.2 | ¹H NMR (400 MHz, DMSO-d₆) |
| | benzylpiperidin-3- | (M + 1), | δ 9.70-9.64 (m, 1H), 9.48-9.42 (m, |
| | yl)oxy)-N-(6- | 447.2 | 1H), 8.88 (d, J = 2.8 Hz, 1H), 8.50- |
| | chloropyridin-3- | (M + 1). | 8.41 (m, 2H), 7.98 (d, J = 5.7 Hz, 1H), |
| | yl)isoquinolin-1-amine | | 7.56-7.46 (m, 5H), 7.37-7.29 (m, 2H), |
| | | | 7.16 (d, J = 5.9 Hz, 1H), 5.10 (dd, J = |
| | | | 1.4, 0.7 Hz, 1H), 4.47-4.31 (m, 2H), |
| | | | 3.56-3.27 (m, 2H), 3.11-2.99 (m, 2H), |
| | | | 2.06-2.00 (m, 2H), 1.85-1.74 (m, 2H). |
| 109 | N-(6-chloropyridin-3- | 399.2 | ¹H NMR (400 MHz, DMSO-d₆) δ 9.37 |
| | yl)-6-((1- | (M + 1), | (s, 1H), 8.88 (d, J = 2.8 Hz, 1H), 8.44- |
| | morpholinopropan-2- | 401.2 | 8.41 (m, 2H), 7.95 (d, J = 5.7 Hz, 1H), |
| | yl)oxy)isoquinolin-1- | (M + 1). | 7.45 (d, J = 8.7 Hz, 1H), 7.35-7.34 (m, |
| | amine | | 1H), 7.27-7.24 (m, 1H), 7.17-7.16 (m, |
| | | | 1H), 4.91-4.87 (m, 1H), 3.56-3.54 (m, |
| | | | 4H), 2.68-2.62 (m, 1H), 2.54-2.52 (m, |
| | | | 1H), 2.49-2.45 (m, 4H), 1.33 (d, J = |
| | | | 6.1 Hz, 3H). |
| 110 | ethyl 1-(((1-((6- | 398.2 | ¹H NMR (400 MHz, DMSO-d₆) δ 9.38 |
| | chloropyridin-3- | (M + 1), | (s, 1H), 8.89 (d, J = 2.8 Hz, 1H), 8.44 |
| | yl)amino)isoquinolin-6- | 400.2 | (dd, J = 8.8, 2.8 Hz, 2H), 7.97 (d, J = |
| | yl)oxy)methyl)cyclopropane- | (M + 1). | 5.8 Hz, 1H), 7.45 (d, J = 8.8 Hz, 1H), |
| | 1-carboxylate | | 7.32-7.27 (m, 2H), 7.17 (d, J = 5.8 Hz, |
| | | | 1H), 4.26 (s, 2H), 4.09 (q, J = 7.1 Hz, |
| | | | 2H), 1.29 (q, J = 3.5 Hz, 2H), 1.16- |
| | | | 1.12 (m, 5H). |

### Example 111

### Synthesis of (S)-1-(3-((1-((6-chloropyridin-3-yl)amino)isoquinolin-6-yl)oxy)pyrrolidin-1-yl)ethan-1-one

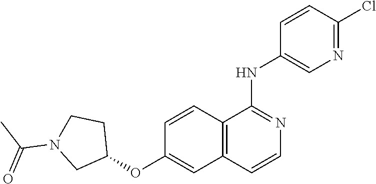 [SMILES: CC(=O)N1CC[C@H](Oc2ccc3c(Nc4ccc(Cl)nc4)nccc3c2)C1]

### Step 1. Preparation of (S)—N-(6-chloropyridin-3-yl)-6-(pyrrolidin-3-yloxy)isoquinolin-1-amine

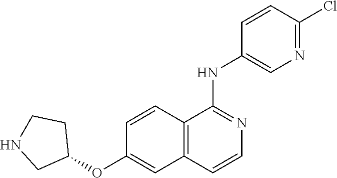 [SMILES: Clc1ccc(Nc2nccc3cc(O[C@H]4CCNC4)ccc23)cn1]

To a solution of tert-butyl (S)-3-hydroxypyrrolidine-1-carboxylate (0.056 g, 0.297 mmol) in N,N-dimethylformamide (6 mL) was added sodium hydride (60% dispersion in mineral oil, 0.018 g, 0.446 mmol) at 0° C. and the resulting mixture was stirred for 20 minutes at this temperature. To the reaction mixture was then added N-(6-chloropyridin-3-yl)-6-fluoro-N-((2-(trimethylsilyl)ethoxy)methyl)isoquinolin-1-amine (0.060 g, 0.149 mmol) at 0° C. The reaction mixture was allowed to warm to ambient temperature and then heated to 60° C. for 16 h. The mixture was quenched by addition of water (20 mL). After dilution with ethyl acetate (20 mL), the organic phase was washed with saturated aqueous ammonium chloride (20 mL) and brine (20 mL). The organic phase was dried over anhydrous sodium sulfate, filtered, and the filtrate was concentrated in vacuo. The obtained residue was dissolved in dichloromethane (3 mL). To this mixture was then added trifluoroacetic acid (0.228 mL, 2.98 mmol) at ambient temperature and the reaction mixture was stirred at ambient temperature for 1 h. The reaction mixture was concentrated in vacuo to afford the title compound as a colorless oil (0.050 g, 50% yield): MS (ES+) m/z 341.2 (M+1), 343.2 (M+1).

### Step 2. Preparation of (S)-1-(3-((1-((6-chloropyridin-3-yl)amino)isoquinolin-6-yl)oxy)pyrrolidin-1-yl)ethan-1-one

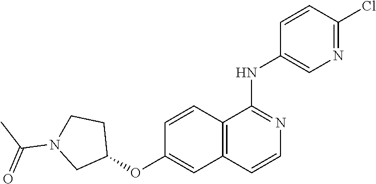 [SMILES: CC(=O)N1CC[C@H](Oc2ccc3c(Nc4ccc(Cl)nc4)nccc3c2)C1]

To a solution of (S)—N-(6-chloropyridin-3-yl)-6-(pyrrolidin-3-yloxy)isoquinolin-1-amine (0.050 g, 0.147 mmol) in dichloromethane (5 mL), was added triethylamine (0.10 mL, 0.725 mmol) and acetyl chloride (0.023 g, 0.294 mmol). The reaction mixture was stirred at ambient temperature 30 minutes and then concentrated in vacuo. The obtained residue was purified by silica gel column chromatography, eluting with a gradient of 0 to 60% of ethyl acetate in heptane, to provide the title compound as a colorless solid (0.022 g, 18% yield): ¹H NMR (400 MHz, DMSO-d₆) δ 9.40 (s, 1H), 8.88 (s, 1H), 8.47-8.42 (m, 2H), 7.98-7.97 (m, 1H), 7.45 (d, J=8.7 Hz, 1H), 7.36-7.27 (m, 2H), 7.21-7.19 (m, 1H), 5.31-5.20 (m, 1H), 3.92-3.56 (m, 4H), 2.35-2.08 (m, 2H), 2.00 (s, 3H); MS (ES+) m/z 383.2 (M+1), 385.2 (M+1).

### Example 112

### Synthesis of (R)-1-(3-((1-((6-chloropyridin-3-yl)amino)isoquinolin-6-yl)oxy)pyrrolidin-1-yl)ethan-1-one

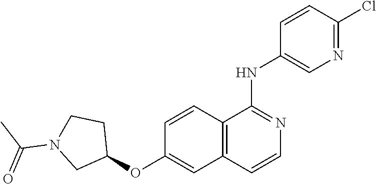 [SMILES: CC(=O)N1CC[C@@H](Oc2ccc3c(Nc4ccc(Cl)nc4)nccc3c2)C1]

Following the procedure as described for EXAMPLE 87, Step 1 and 2 and making variations as required to replace tert-butyl (S)-3-hydroxypyrrolidine-1-carboxylate with tert-butyl (R)-3-hydroxypyrrolidine-1-carboxylate, the title compound was obtained as a colorless solid (0.0269 g, 10% yield): ¹H NMR (400 MHz, DMSO-d₆) δ 9.53 (s, 1H), 8.87 (s, 1H), 8.47 (d, J=9.2 Hz, 1H), 8.42-8.39 (m, 1H), 7.95 (dd, J=5.8, 2.0 Hz, 1H), 7.47 (d, J=8.7 Hz, 1H), 7.36 (dd, J=7.1, 2.5 Hz, 1H), 7.35-7.28 (m, 1H), 7.21 (dd, J=5.8, 2.6 Hz, 1H), 5.31-5.21 (m, 1H), 3.67-3.55 (m, 4H), 2.35-2.12 (m, 2H), 2.00 (s, 3H); MS (ES+) m/z 383.2 (M+1), 385.2 (M+1).

### Example 113

### Synthesis of 6-((2-oxaspiro[3.3]heptan-6-yl)oxy)-N-(6-chloropyridin-3-yl)isoquinolin-1-amine

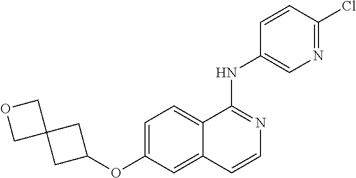 [SMILES: Clc1ccc(Nc2nccc3cc(OC4CC5(COC5)C4)ccc23)cn1]

To the mixture of 1-((6-chloropyridin-3-yl)amino)isoquinolin-6-ol (0.080 g, 0.294 mmol,) and potassium carbonate (0.049 g, 0.353 mmol) in N,N-dimethylformamide (2 mL) was added 6-iodo-2-oxaspiro[3.3]heptane (0.079 g, 0.353 mmol). The reaction mixture was stirred at 80° C. for 4 h. After cooling to ambient temperature, the reaction mixture was diluted with ethyl acetate (20 mL) and washed with water (20 mL) and brine (20 mL). The organic phase was dried over anhydrous sodium sulfate and filtered. The filtrate was concentrated in vacuo to give a residue. The obtained residue was first purified by silica gel column chromatography, eluting with a gradient of 0 to 100% of ethyl acetate in heptane, and then purified by preparative HPLC, eluting with a gradient of 20 to 60% of acetonitrile in water containing 0.5% of formic acid, to provide the title compound as a colorless solid (0.0093 g, 8% yield): ¹H NMR (400 MHz, CDCl₃) δ 8.51 (d, J=2.4 Hz, 1H), 8.30-8.28 (m, 1H), 7.97 (td, J=0.5, 0.3 Hz, 2H), 7.31 (d, J=8.5 Hz, 1H), 7.15 (dd, J=9.1, 2.4 Hz, 1H), 7.11 (d, J=6.0 Hz, 1H), 6.90 (d, J=2.3 Hz, 1H), 4.82 (s, 2H), 4.75 (s, 2H), 4.72-4.67 (m, 1H), 2.95-2.90 (m, 2H), 2.49-2.44 (m, 2H), NH not observed; MS (ES+) m/z 368.0 (M+1), 370.0 (M+1).

### Example 114

### Synthesis of 6-((1-oxaspiro[3.3]heptan-6-yl)methoxy)-N-(6-chloropyridin-3-yl)isoquinolin-1-amine

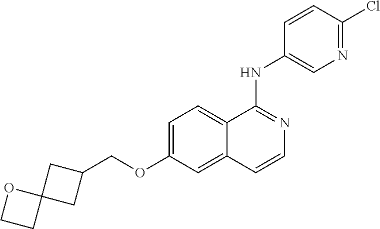 [SMILES: Clc1ccc(Nc2nccc3cc(OCC4CC5(CCO5)C4)ccc23)cn1]

To the mixture of 1-((6-chloropyridin-3-yl)amino)isoquinolin-6-ol (0.080 g, 0.294 mmol,) and potassium carbonate (0.049 g, 0.589 mmol) in N,N-dimethylformamide (2 mL) was added 6-(bromomethyl)-1-oxaspiro[3.3]heptane (0.113 g, 0.589 mmol) and the reaction mixture was heated to 80° C. for 2 h. After cooling to ambient temperature, the reaction mixture was diluted with ethyl acetate (20 mL) and washed with water (20 mL) and brine (20 mL). The organic phase was dried over anhydrous sodium sulfate and filtered. The filtrate was concentrated in vacuo to give a residue. The obtained residue was purified by silica gel column chromatography, eluting with a gradient of 0 to 100% of ethyl acetate in heptane, to provide the title compound as a colorless solid (0.0442 g, 39% yield): ¹H NMR (400 MHz, DMSO-d₆) δ9.37 (s, 1H), 8.89 (d, J=2.8 Hz, 1H), 8.43 (dd, J=8.8, 2.7 Hz, 2H), 7.96 (dd, J=5.8, 2.1 Hz, 1H), 7.45 (d, J=8.7 Hz, 1H), 7.28-7.25 (m, 2H), 7.17 (dd, J=5.6, 4.0 Hz, 1H), 4.38 (td, J=7.5, 3.2 Hz, 2H), 4.08 (dd, J=11.9, 6.6 Hz, 2H), 2.70 (q, J=7.4 Hz, 2H), 2.47-2.41 (m, 2H), 2.25 (dd, J=13.8, 3.6 Hz, 2H), 2.08-2.02 (m, 1H); MS (ES+) m/z 382.0 (M+1), 384.0 (M+1).

### Example 115

### Synthesis of N-(6-chloropyridin-3-yl)-6-((tetrahydro-1H-pyrrolizin-7a(5H)-yl)methoxy)isoquinolin-1-amine

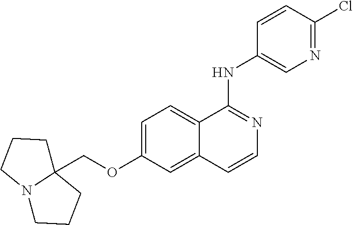 [SMILES: Clc1ccc(Nc2nccc3cc(OCC45CCCN4CCC5)ccc23)cn1]

### Step 1. Preparation of N-(6-chloropyridin-3-yl)-6-fluoroisoquinolin-1-amine

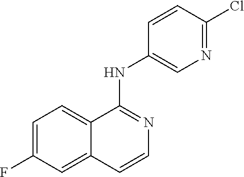 [SMILES: Fc1ccc2c(Nc3ccc(Cl)nc3)nccc2c1]

To a mixture of 1-chloro-6-fluoroisoquinoline (0.050 g, 0.275 mmol), 6-chloropyridin-3-amine (0.039 g, 0.303 mmol) and potassium phosphate tribasic (0.234 g, 1.10 mmol) in 1,4-dioxane (4 mL) were added dicyclohexyl(2′,6′-dimethoxy-[1,1′-biphenyl]-2-yl)phosphane (0.023 g, 0.055 mmol) and tris(dibenzylideneacetone)dipalladium (0.025 g, 0.028 mmol). The resulting mixture was degassed by passing a stream of nitrogen through it for 5 minutes and then heated to 120° C. in microwave reactor for 1 h. After cooling to ambient temperature, the reaction mixture was filtered through celite, and the filtrate was concentrated in vacuo to give a residue. The residue was purified by silica gel column chromatography, eluting with a gradient of 0 to 100% of ethyl acetate in heptane, to provide the title compound as a colorless solid (0.0450 g, 41% yield): MS (ES+) m/z 274.6 (M+1), 276.6 (M+1).

### Step 2. Preparation of N-(6-chloropyridin-3-yl)-6-fluoro-N-(4-methoxybenzyl)isoquinolin-1-amine

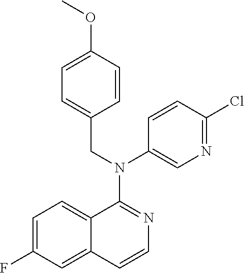 [SMILES: COc1ccc(CN(c2ccc(Cl)nc2)c2nccc3cc(F)ccc23)cc1]

To a solution of N-(6-chloropyridin-3-yl)-6-fluoroisoquinolin-1-amine (0.600 g, 1.86 mmol) in N,N-dimethylformamide (10 mL) was added sodium hydride (60 wt. % dispersion in mineral oil, 0.112 g, 2.80 mmol) at ambient temperature. The reaction mixture was stirred for 10 minutes, then 4-methoxybenzyl chloride (0.38 mL, 2.80 mmol) was added to it. The resulting mixture was stirred at ambient temperature for 2 h and then diluted with ethyl acetate (20 mL). The mixture was washed with saturated sodium bicarbonate (20 mL) and brine (20 mL). The organic phase was dried over anhydrous sodium sulfate and filtered. The filtrate was concentrated in vacuo to give a residue. The residue was purified by silica gel column chromatography, eluting with a gradient of 0 to 30% of ethyl acetate in heptane, to provide the title compound as yellow oil (0.495 g, 67% yield): ¹H NMR (400 MHz, DMSO-d₆) δ 8.37 (d, J=5.7 Hz, 1H), 7.88 (d, J=2.9 Hz, 1H), 7.80 (dt, J=9.5, 4.7 Hz, 2H), 7.67 (d, J=5.7 Hz, 1H), 7.43-7.40 (m, 3H), 7.27 (d, J=8.7 Hz, 1H), 7.20 (dd, J=8.7, 3.0 Hz, 1H), 6.85-6.83 (m, 2H), 5.26 (s, 2H), 3.69 (s, 3H).

### Step 3. Preparation of N-(6-chloropyridin-3-yl)-N-(4-methoxybenzyl)-6-((tetrahydro-1H-pyrrolizin-7a(5H)-yl)methoxy)isoquinolin-1-amine

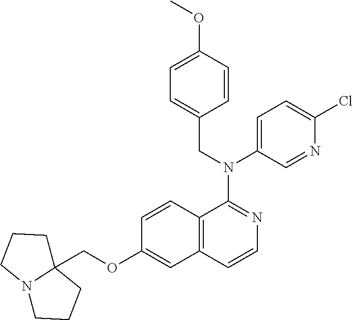 [SMILES: COc1ccc(CN(c2ccc(Cl)nc2)c2nccc3cc(OCC45CCCN4CCC5)ccc23)cc1]

To a solution of (tetrahydro-1H-pyrrolizin-7a(5H)-yl)methanol (0.108 g, 0.762 mmol) in anhydrous N,N-dimethylformamide (1 mL) was added sodium hydride (60 wt. % dispersion in mineral oil, 0.030 g, 0.762 mmol, 3.0 eq) at ambient temperature. The resulting mixture was stirred for 15 minutes, and then added a solution of N-(6-chloropyridin-3-yl)-6-fluoro-N-(4-methoxybenzyl)isoquinolin-1-amine (0.100 g, 0.254 mmol, 1.0 eq) in anhydrous N,N-dimethylformamide (2 mL) was added to it. The reaction mixture was stirred at ambient temperature for 30 minutes. The mixture was diluted with ethyl acetate (20 mL) and washed with saturated sodium bicarbonate (20 mL) and brine (20 mL). The organic phase was dried over anhydrous sodium sulfate and filtered. The filtrate was concentrated in vacuo to give a residue. The residue was purified by silica gel column chromatography, eluting with a gradient of 5% to 100% ethyl acetate in heptane, followed by a gradient of 0 to 20% of methanol in dichloromethane, to provide the title compound as a yellow oil (0.102 g, 77% yield): ¹H NMR (400 MHz, DMSO-d₆) δ 8.28 (d, J=5.7 Hz, 1H), 7.81 (d, J=2.9 Hz, 1H), 7.58 (dd, J=15.6, 7.6 Hz, 2H), 7.42-7.40 (m, 3H), 7.23 (d, J=8.7 Hz, 1H), 7.13 (dd, J=9.3, 2.6 Hz, 1H), 7.10 (dd, J=8.8, 3.1 Hz, 1H), 6.84 (d, J=8.7 Hz, 2H), 3.77 (s, 2H), 3.69 (s, 3H), 3.34 (d, J=0.3 Hz, 2H), 2.97-2.92 (m, 2H), 2.59-2.53 (m, 2H), 1.92-1.73 (m, 6H), 1.62-1.57 (m, 2H); MS (ES+) m/z 515.6 (M+1), 517.6 (M+1).

### Step 4. Preparation of N-(6-chloropyridin-3-yl)-6-((tetrahydro-1H-pyrrolizin-7a(5H)-yl)methoxy)isoquinolin-1-amine

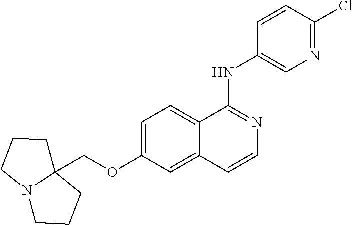 [SMILES: Clc1ccc(Nc2nccc3cc(OCC45CCCN4CCC5)ccc23)cn1]

A mixture of N-(6-chloropyridin-3-yl)-N-(4-methoxybenzyl)-6-((tetrahydro-1H-pyrrolizin-7a(5H)-yl)methoxy)isoquinolin-1-amine (0.050 g, 0.097 mmol) and trifluoroacetic acid (1 mL, 10.1 mmol) in dichloromethane (1 mL) was heated to 50° C. for 16 h. After cooling to ambient temperature, the mixture was diluted with ethyl acetate (20 mL) and washed with saturated sodium bicarbonate (20 mL) and brine (20 mL). The organic phase was dried over anhydrous sodium sulfate and filtered. The filtrate was concentrated in vacuo to give a residue. The residue was purified by reverse-phase preparative HPLC, eluting with a gradient of 15 to 30% of acetonitrile in water containing 0.5% of formic acid, to provide the title compound as a colorless solid (0.0192 g, 50% yield): ¹H NMR (400 MHz, DMSO-d₆) δ 9.51 (s, 1H), 8.92 (d, J=2.8 Hz, 1H), 8.55 (d, J=9.3 Hz, 1H), 8.46 (dd, J=8.8, 2.9 Hz, 1H), 7.99 (d, J=5.8 Hz, 1H), 7.45 (d, J=8.7 Hz, 1H), 7.37-7.31 (m, 2H), 7.18 (d, J=5.8 Hz, 1H), 4.32 (s, 2H), 3.54-3.48 (m, 2H), 3.21-3.15 (m, 2H), 2.21-1.97 (m, 8H); MS (ES+) m/z 395.2 (M+1), 397.2 (M+1).

### Example 116

### Synthesis of N-(6-chloropyridin-3-yl)-6-(2-(3-fluoroazetidin-1-yl)ethoxy)isoquinolin-1-amine

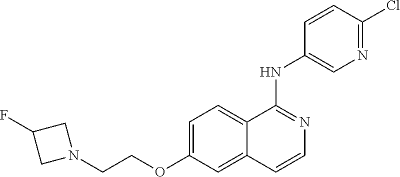 [SMILES: FC1CN(CCOc2ccc3c(Nc4ccc(Cl)nc4)nccc3c2)C1]

Following the procedure as described for EXAMPLE 76, Step 3 and making variations as required to replace phenylmethanol with 2-(3-fluoroazetidin-1-yl)ethan-1-ol, the title compound was obtained as a colorless solid (0.023 g, 41% yield): ¹H NMR (400 MHz, DMSO-d₆) δ9.38 (s, 1H), 8.88 (d, J=2.8 Hz, 1H), 8.42 (dd, J=8.7, 2.9 Hz, 2H), 7.96 (d, J=5.8 Hz, 1H), 7.45 (d, J=8.7 Hz, 1H), 7.26 (t, J=3.4 Hz, 1H), 7.18 (d, J=5.8 Hz, 1H), 5.27-5.07 (m, 1H), 4.12 (t, J=5.4 Hz, 2H), 3.70-3.62 (m, 2H), 3.23 (ddd, J=23.9, 9.6, 4.6 Hz, 2H), 2.89 (t, J=5.4 Hz, 2H); ¹⁹F NMR (376 MHz, DMSO-d₆) δ −177.76 (s, 1F); MS (ES+) m/z 373.0 (M+1), 375.0 (M+1).

### Example 117

### Synthesis of N-(6-chloropyridin-3-yl)-6-((1-(oxetan-3-yl)-1H-pyrazol-4-yl)methoxy)isoquinolin-1-amine

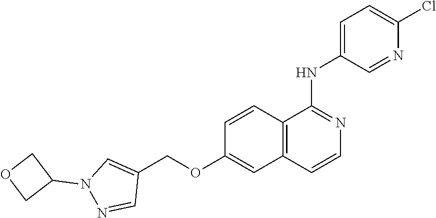 [SMILES: Clc1ccc(Nc2nccc3cc(OCc4cnn(C5COC5)c4)ccc23)cn1]

To a solution of (1-(oxetan-3-yl)-1H-pyrazol-4-yl)methanol (0.057 g, 0.371 mmol) in dichloromethane (1 mL) was added sodium hydride (60% dispersion in mineral oil, 0.015 g, 0.371 mmol) at ambient temperature and the resulting mixture was stirred for 30 minutes at this temperature. To the reaction mixture was then added a solution of N-(6-chloropyridin-3-yl)-6-fluoro-N-((2-(trimethylsilyl)ethoxy)methyl)isoquinolin-1-amine (0.050 g, 0.124 mmol) in N,N-dimethylformamide (1 mL) and the reaction mixture was stirred at ambient temperature for 16 h. The reaction mixture was diluted with ethyl acetate (20 mL) and washed with saturated bicarbonate (20 mL) and brine (20 mL). The organic phase was washed with saturated sodium bicarbonate (20 mL) and brine (20 mL), and dried over anhydrous sodium sulfate and filtered. The filtrate was concentrated in vacuo to give a residue. To the obtained residue was added dichloromethane (1 mL) and trifluoroacetic acid (0.8 mL, 10.8 mmol) at ambient temperature. The reaction mixture was stirred at ambient temperature for 3 h and then concentrated in vacuo. The obtained residue was purified by silica gel chromatography, eluting with a gradient of 0 to 100% of ethyl acetate in heptane, followed by a gradient of 0 to 5% of methanol in dichloromethane, to provide the title compound as a colorless solid (0.012 g, 23% yield): ¹H NMR (400 MHz, DMSO-d₆) δ 9.38 (s, 1H), 8.88 (d, J=2.8 Hz, 1H), 8.43 (td, J=5.7, 3.4 Hz, 2H), 8.08 (s, 1H), 7.97 (d, J=5.8 Hz, 1H), 7.76 (s, 1H), 7.45 (d, J=8.7 Hz, 1H), 7.42 (d, J=2.5 Hz, 1H), 7.28 (dd, J=9.2, 2.5 Hz, 1H), 7.21 (d, J=5.7 Hz, 1H), 5.63-5.56 (m, 1H), 5.14 (s, 2H), 4.90 (quintet, J=6.7 Hz, 4H); MS (ES+) m/z 408.0 (M+1), 410.0 (M+1).

### Example 118

### Synthesis of N-(6-chloropyridin-3-yl)-6-((1-(methylsulfonyl)piperidin-4-yl)oxy)isoquinolin-1-amine

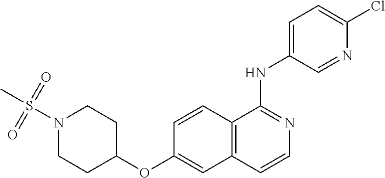 [SMILES: CS(=O)(=O)N1CCC(Oc2ccc3c(Nc4ccc(Cl)nc4)nccc3c2)CC1]

### Step 1. Preparation of N-(6-chloropyridin-3-yl)-6-(piperidin-4-yloxy)isoquinolin-1-amine

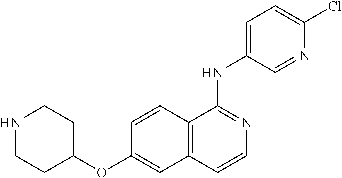 [SMILES: Clc1ccc(Nc2nccc3cc(OC4CCNCC4)ccc23)cn1]

Following the procedure as described for EXAMPLE 76, Step 3 and making variations as required to replace phenylmethanol with tert-butyl 4-hydroxy-1-piperidinecarboxylate, the title compound was obtained as a yellow oil (0.058 g, 100% yield), that was used in the next step without purification.

### Step 2. Preparation of N-(6-chloropyridin-3-yl)-6-((1-(methylsulfonyl)piperidin-4-yl)oxy)isoquinolin-1-amine

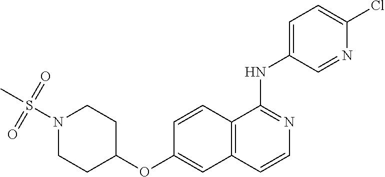 [SMILES: CS(=O)(=O)N1CCC(Oc2ccc3c(Nc4ccc(Cl)nc4)nccc3c2)CC1]

To a solution of N-(6-chloropyridin-3-yl)-6-(piperidin-4-yloxy)isoquinolin-1-amine (0.058 g, 0.163 mmol) in dichloromethane (1 mL) was added triethylamine (0.068 mL, 0.490 mmol) and methanesulfonyl chloride (0.014 mL, 0.180 mmol) at ambient temperature. The reaction mixture was stirred at ambient temperature for 30 minutes, and then concentrated in vacuo to afford a residue. The obtained residue was purified by reverse-phase preparative HPLC, eluting with a gradient of 44 to 64% of acetonitrile in water containing 10 mM ammonium formate, to provide the title compound as a colorless solid (0.016 g, 21% yield): ¹H NMR (400 MHz, DMSO-d₆) δ 9.40 (s, 1H), 8.88 (d, J=2.8 Hz, 1H), 8.47-8.45 (m, 1H), 8.42 (t, J=4.4 Hz, 1H), 7.97 (d, J=5.8 Hz, 1H), 7.45 (d, J=8.7 Hz, 1H), 7.38 (d, J=2.5 Hz, 1H), 7.31 (dd, J=9.2, 2.5 Hz, 1H), 7.17 (d, J=5.8 Hz, 1H), 4.83-4.77 (m, 1H), 3.43-3.39 (m, 2H), 3.21-3.15 (m, 2H), 2.94 (s, 3H), 2.13-2.08 (m, 2H), 1.87-1.78 (m, 2H); MS (ES+) m/z 434.0 (M+1), 436.0 (M+1).

### Example 119

### Synthesis of N-(6-chloropyridin-3-yl)-6-((1-(2-methoxyethyl)piperidin-4-yl)oxy)isoquinolin-1-amine

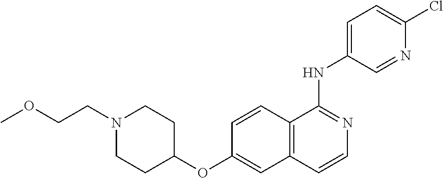 [SMILES: COCCN1CCC(Oc2ccc3c(Nc4ccc(Cl)nc4)nccc3c2)CC1]

To a solution of N-(6-chloropyridin-3-yl)-6-(piperidin-4-yloxy)isoquinolin-1-amine (0.058 g, 0.163 mmol) in N,N-dimethylformamide (1 mL) was added potassium carbonate (0.068 g, 0.490 mmol) and 1-bromo-2-methoxyethane (0.030 mL, 0.327 mmol) at ambient temperature. The reaction mixture was heated to 50° C. for 30 minutes. After cooling to ambient temperature, the mixture was concentrated in vacuo to afford a residue. The obtained residue was purified by reverse-phase preparative HPLC, eluting with a gradient of 10 to 30% of acetonitrile in water (containing 10 mM ammonium formate), to provide the title compound as a colorless solid (0.018 g, 26% yield): ¹H NMR (400 MHz, DMSO-d₆) δ 9.38 (d, J=4.8 Hz, 1H), 8.89-8.88 (m, 1H), 8.45-8.41 (m, 2H), 7.95-7.94 (m, 1H), 7.45 (d, J=8.8 Hz, 1H), 7.33 (dd, J=3.0, 1.7 Hz, 1H), 7.28-7.25 (m, 1H), 7.18 (d, J=5.9 Hz, 1H), 4.62-4.57 (m, 1H), 3.48-3.43 (m, 4H), 3.26-3.22 (m, 3H), 2.80-2.73 (m, 2H), 2.41-2.29 (m, 2H), 2.06-1.99 (m, 2H), 1.72-1.64 (m, 2H); MS (ES+) m/z 414.2 (M+1), 416.2 (M+1).

### Example 120

### Synthesis of 6-(2-(1H-pyrazol-1-yl)ethoxy)-N-(6-chloropyridin-3-yl)isoquinolin-1-amine

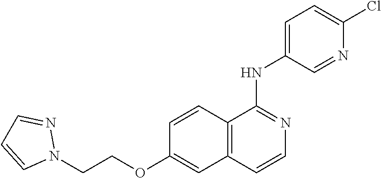 [SMILES: Clc1ccc(Nc2nccc3cc(OCCn4cccn4)ccc23)cn1]

### Step 1. Preparation of 6-(2-bromoethoxy)-1-chloroisoquinoline

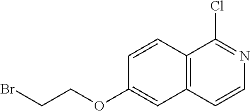 [SMILES: Clc1nccc2cc(OCCBr)ccc12]

To a solution of 1-chloroisoquinolin-6-ol (0.100 g, 0.557 mmol) in N,N-dimethylformamide (5 mL) was added sodium hydride (60% dispersion in mineral oil, 0.022 g, 0.557 mmol) at 0° C. The reaction mixture was stirred at ambient temperature for 30 minutes. To the reaction mixture was then added 1,2-dibromoethane (0.053 mL, 0.612 mmol) and the resulting mixture was stirred at ambient temperature for 1 h. Another portion of sodium hydride (60% dispersion in mineral oil, 0.022 g, 0.557 mmol) and 1,2-dibromoethane (0.053 mL, 0.612 mmol) was added to the reaction mixture, and the mixture was stirred for 3 h. This process was repeated three times. The reaction mixture was then diluted with ethyl acetate (20 mL) and washed with saturated ammonium chloride (20 mL). The organic phase was dried over anhydrous sodium sulfate and filtered. The filtrate was concentrated in vacuo. The obtained residue was purified by silica gel column chromatography, eluting with a gradient of 0 to 50% of ethyl acetate in heptane, to provide the title compound as a colorless solid (0.091 g, 55% yield): MS (ES+) m/z 286.5 (M+1), 288.5 (M+1).

### Step 2. Preparation of 6-(2-(1H-pyrazol-1-yl)ethoxy)-1-chloroisoquinoline

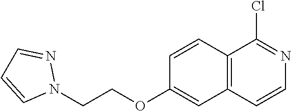 [SMILES: Clc1nccc2cc(OCCn3cccn3)ccc12]

To a solution of pyrazole (0.018 g, 0.262 mmol) in tetrahydrofuran (2 mL) was added sodium hydride (60% dispersion in mineral oil, 0.010 g, 0.262 mmol) at 0° C. The reaction mixture was allowed to warm to ambient temperature and stirred for 30 minutes. To the reaction mixture was then added a solution of 6-(2-bromoethoxy)-1-chloroisoquinoline (0.050 g, 0.174 mmol) in tetrahydrofuran (2 mL). The resulting mixture was stirred at ambient temperature for 16 h. The reaction mixture was diluted with ethyl acetate (20 mL) and washed with saturated sodium bicarbonate (20 mL) and brine (20 mL). The organic phase was dried over anhydrous sodium sulfate and filtered. The filtrate was concentrated in vacuo to give a residue. The residue was purified by silica gel column chromatography, eluting with a gradient of 0 to 100% of ethyl acetate in heptane, to provide the title compound as a yellow oil (0.008 g, 20% yield): MS (ES+) m/z 274.6 (M+1), 276.6 (M+1).

### Step 3. Preparation of 6-(2-(1H-pyrazol-1-yl)ethoxy)-N-(6-chloropyridin-3-yl)isoquinolin-1-amine

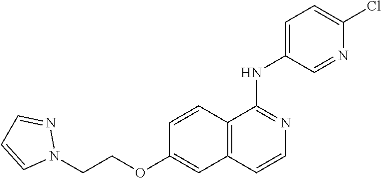 [SMILES: Clc1ccc(Nc2nccc3cc(OCCn4cccn4)ccc23)cn1]

To a mixture of 6-(2-(1H-pyrazol-1-yl)ethoxy)-1-chloroisoquinoline (0.008 g, 0.035 mmol), 6-chloropyridin-3-amine (0.005 g, 0.038 mmol) and potassium phosphate tribasic (0.029 g, 0.139 mmol) in 1,4-dioxane (5 mL) were added dicyclohexyl(2′,6′-dimethoxy-[1,1′-biphenyl]-2-yl)phosphane (0.003 g, 0.007 mmol) and tris(dibenzylideneacetone)dipalladium (0.003 g, 0.003 mmol). The resulting mixture was degassed by passing a stream of nitrogen through it for 5 minutes and then heated to 100° C. for 2 h. After cooling to ambient temperature, the reaction mixture was filtered through celite and the filtrate was concentrated in vacuo to give a residue. The obtained residue was purified by reverse-phase preparative HPLC, eluting with a gradient of 40 to 60% of acetonitrile in water (containing 10 mM ammonium formate), to provide the title compound as a colorless solid (0.005 g, 34% yield): ¹H NMR (400 MHz, DMSO-d₆) δ 9.41 (s, 1H), 8.88 (d, J=2.6 Hz, 1H), 8.44-8.41 (m, 2H), 7.96 (d, J=5.7 Hz, 1H), 7.84 (d, J=2.1 Hz, 1H), 7.49 (m, 1H), 7.45 (d, J=8.8 Hz, 1H), 7.30 (m, 1H), 7.25-7.22 (m, 1H), 7.17 (d, J=5.9 Hz, 1H), 6.27 (s, 1H), 4.60-4.57 (m, 2H), 4.52-4.49 (m, 2H); MS (ES+) m/z 366.2 (M+1), 368.0 (M+1).

### Example 121

### Synthesis of (4-((1-((6-chloropyridin-3-yl)amino)isoquinolin-6-yl)oxy)piperidin-1-yl)(cyclopropyl)methanone

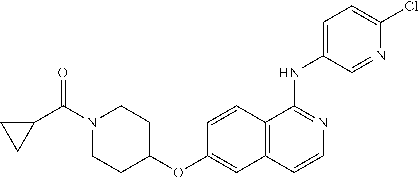 [SMILES: O=C(C1CC1)N1CCC(Oc2ccc3c(Nc4ccc(Cl)nc4)nccc3c2)CC1]

To a mixture of N-(6-chloropyridin-3-yl)-6-(piperidin-4-yloxy)isoquinolin-1-amine (0.058 g, 0.163 mmol), cyclopropanecarboxylic acid (0.017 g, 0.196 mmol), and diisopropylamine (0.067 mL, 0.490 mmol) in dichloromethane (1 mL) was added 1-[bis(dimethylamino)methylene]-1H-1,2,3-triazolo[4,5-b]pyridinium 3-oxide hexafluorophosphate (0.075 mg, 0.196 mmol). The reaction mixture was stirred at ambient temperature for 3 days. The reaction mixture was diluted with ethyl acetate (20 mL) and washed with water (20 mL) and brine (20 mL). The organic phase was dried over anhydrous sodium sulfate and filtered. The filtrate was concentrated in vacuo to give a residue. The obtained residue was purified by reverse-phase preparative HPLC, eluting with a gradient of 45 to 65% of acetonitrile in water (containing 10 mM ammonium formate), to provide the title compound as a colorless solid (0.015 g, 20% yield): ¹H NMR (400 MHz, DMSO-d₆) δ 9.39 (s, 1H), 8.88 (d, J=2.8 Hz, 1H), 8.46-8.42 (m, 2H), 7.96 (d, J=5.8 Hz, 1H), 7.45 (d, J=8.8 Hz, 1H), 7.40 (d, J=2.6 Hz, 1H), 7.31 (dd, J=9.2, 2.5 Hz, 1H), 7.18 (d, J=5.9 Hz, 1H), 4.90-4.85 (m, 1H), 4.07-4.02 (m, 1H), 3.98-3.92 (m, 1H), 3.61-3.55 (m, 1H), 3.46-3.42 (m, 1H), 2.15-2.11 (m, 1H), 2.05-2.00 (m, 2H), 1.72-1.67 (m, 1H), 1.62-1.55 (m, 1H), 0.75-0.71 (m, 4H); MS (ES+) m/z 424.2 (M+1), 426.0 (M+1).

### Example 122

### Synthesis of (S)-1-(3-((1-((6-chloropyridin-3-yl)amino)isoquinolin-6-yl)oxy)piperidin-1-yl)ethan-1-one

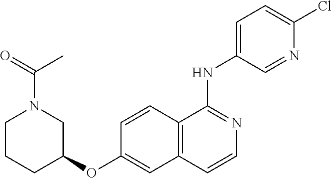 [SMILES: CC(=O)N1CCC[C@H](Oc2ccc3c(Nc4ccc(Cl)nc4)nccc3c2)C1]

### Step 1: Preparation of (S)—N-(6-chloropyridin-3-yl)-6-(piperidin-3-yloxy)isoquinolin-1-amine

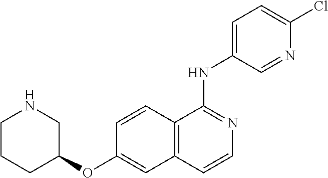 [SMILES: Clc1ccc(Nc2nccc3cc(O[C@H]4CCCNC4)ccc23)cn1]

Following the procedure as described for EXAMPLE 76, Step 3 and making variations as required to replace phenylmethanol with (S)-1-Boc-3-hydroxypiperidine, the title compound was obtained as a yellow oil. The obtained residue was used in the in the next step without purification.

### Step 2: Preparation of (S)-1-(3-((1-((6-chloropyridin-3-yl)amino)isoquinolin-6-yl)oxy)piperidin-1-yl)ethan-1-one

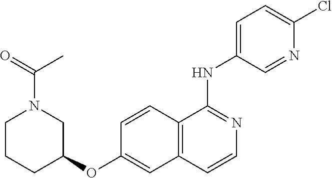 [SMILES: CC(=O)N1CCC[C@H](Oc2ccc3c(Nc4ccc(Cl)nc4)nccc3c2)C1]

To a solution of (S)—N-(6-chloropyridin-3-yl)-6-(piperidin-3-yloxy)isoquinolin-1-amine (0.072 g, 0.123 mmol) in dichloromethane (2 mL) was added triethylamine (0.086 mL, 0.615 mmol) and acetyl chloride (0.011 mL, 0.148 mmol) at ambient temperature. The reaction was stirred at ambient temperature for 30 minutes, then concentrated in vacuo to afford a residue. The obtained residue was purified by reverse phase preparative HPLC, eluting with a gradient of 24 to 44% of acetonitrile in water (containing 10 mM ammonium formate), to provide the title compound as a colorless solid (0.013 g, 24% yield): ¹H NMR (400 MHz, DMSO-d₆) δ9.41-9.40 (m, 1H), 8.88 (d, J=2.8 Hz, 1H), 8.47-8.41 (m, 2H), 7.96 (d, J=5.7 Hz, 1H), 7.45 (d, J=8.8 Hz, 1H), 7.41-7.34 (m, 1H), 7.29-7.25 (m, 1H), 7.19-7.14 (m, 1H), 4.80-4.75 (m, 0.5H), 4.60-4.54 (m, 0.5H), 4.01-3.96 (m, 0.5H), 3.68-3.67 (m, 2H), 3.58-3.50 (m, 0.5H), 2.09-2.02 (m, 1H), 1.88 (s, 3H), 1.82-1.47 (m, 4H); MS (ES+) m/z 397.0 (M+1), 399.0 (M+1).

### Example 123

### Synthesis of (R)-1-(3-((1-((6-chloropyridin-3-yl)amino)isoquinolin-6-yl)oxy)piperidin-1-yl)ethan-1-one

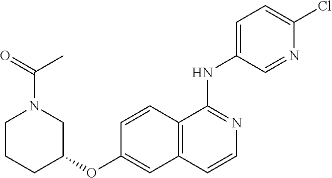 [SMILES: CC(=O)N1CCC[C@@H](Oc2ccc3c(Nc4ccc(Cl)nc4)nccc3c2)C1]

### Step 1: Preparation of (R)—N-(6-chloropyridin-3-yl)-6-(piperidin-3-yloxy)isoquinolin-1-amine

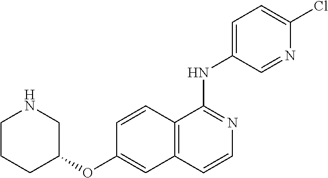 [SMILES: Clc1ccc(Nc2nccc3cc(O[C@@H]4CCCNC4)ccc23)cn1]

Following the procedure as described for EXAMPLE 76, Step 3 and making variations as required to replace phenylmethanol with (R)-1-Boc-3-hydroxypiperidine, the title compound was obtained as a yellow oil. The obtained residue was used in the in the next step without purification.

### Step 2: Preparation of (R)-1-(3-((1-((6-chloropyridin-3-yl)amino)isoquinolin-6-yl)oxy)piperidin-1-yl)ethan-1-one

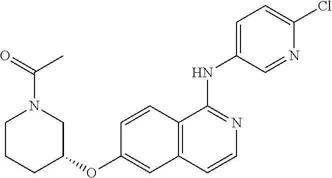 [SMILES: CC(=O)N1CCC[C@@H](Oc2ccc3c(Nc4ccc(Cl)nc4)nccc3c2)C1]

Following the procedure as described for EXAMPLE 122 and making variations as required to replace (S)—N-(6-chloropyridin-3-yl)-6-(piperidin-3-yloxy)isoquinolin-1-amine with (R)—N-(6-chloropyridin-3-yl)-6-(piperidin-3-yloxy)isoquinolin-1-amine, the title compound was obtained as a colorless solid (0.014 g, 28% yield): ¹H NMR (400 MHz, DMSO-d₆) δ 9.41-9.40 (m, 1H), 8.88 (d, J=2.8 Hz, 1H), 8.47-8.42 (m, 2H), 7.96 (d, J=5.8 Hz, 1H), 7.45 (d, J=8.7 Hz, 1H), 7.41-7.34 (m, 1H), 7.31-7.25 (m, 1H), 7.19-7.14 (m, 1H), 4.78-4.77 (m, 0.5H), 4.59-4.55 (m, 0.5H), 4.00-3.96 (m, 0.5H), 3.73-3.66 (m, 2H), 3.58-3.49 (m, 0.5H), 2.12-1.98 (m, 1H), 1.88-1.86 (m, 3H), 1.81-1.46 (m, 4H); MS (ES+) m/z 397.0 (M+1), 399.0 (M+1).

### Example 124

### Synthesis of 3-(((1-((6-chloropyridin-3-yl)amino)isoquinolin-6-yl)oxy)methyl)tetrahydrofuran-3-carbonitrile

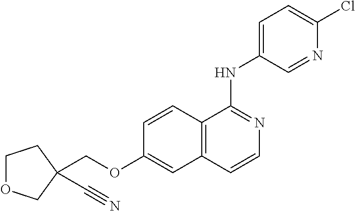 [SMILES: N#CC1(COc2ccc3c(Nc4ccc(Cl)nc4)nccc3c2)CCOC1]

Following the procedure as described for EXAMPLE 76, Step 3 and making variations as required to replace phenylmethanol with 3-(hydroxymethyl)tetrahydrofuran-3-carbonitrile, the title compound was obtained as a colorless solid (0.022 g, 47% yield): ¹H NMR (400 MHz, DMSO-d₆) δ 9.43 (s, 1H), 8.89 (d, J=2.6 Hz, 1H), 8.48 (s, 1H), 8.44 (dd, J=8.8, 2.9 Hz, 1H), 7.99 (d, J=5.8 Hz, 1H), 7.46 (d, J=8.7 Hz, 1H), 7.37 (dd, J=6.7, 2.7 Hz, 2H), 7.19 (d, J=5.8 Hz, 1H), 4.41 (d, J=9.6 Hz, 1H), 4.34 (d, J=9.6 Hz, 1H), 4.10 (d, J=9.2 Hz, 1H), 3.96-3.91 (m, 3H), 2.46-2.41 (m, 1H), 2.26 (dt, J=13.1, 7.3 Hz, 1H); MS (ES+) m/z 381.0 (M+1), 383.0 (M+1).

### Examples 125 AND 126

### Synthesis of (R)-3-(((1-((6-chloropyridin-3-yl)amino)isoquinolin-6-yl)oxy)methyl)tetrahydrofuran-3-carbonitrile and (S)-3-(((1-((6-chloropyridin-3-yl)amino)isoquinolin-6-yl)oxy)methyl)tetrahydrofuran-3-carbonitrile

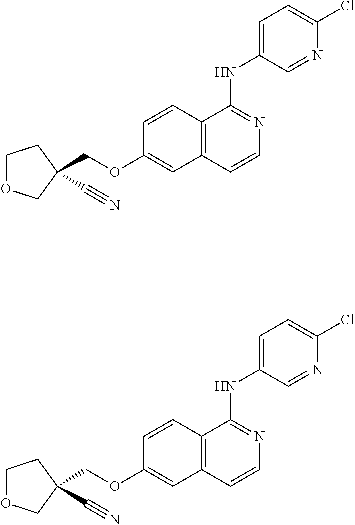 [SMILES: N#C[C@@]1(COc2ccc3c(Nc4ccc(Cl)nc4)nccc3c2)CCOC1 ; N#C[C@]1(COc2ccc3c(Nc4ccc(Cl)nc4)nccc3c2)CCOC1]

Racemic 3-(((1-((6-chloropyridin-3-yl)amino)isoquinolin-6-yl)oxy)methyl)tetrahydrofuran-3-carbonitrile was synthesized as described in EXAMPLE 124. Resolution of the enantiomers by chiral SFC (ChiralPak OJ 250×10 mm, 5 μm column), eluting with 35% of methanol (containing 10 mM ammonium formate) in supercritical carbon dioxide, afforded the title compounds as single enantiomers as colorless solids. First eluting enantiomer (0.046 g, 16% yield): ¹H NMR (400 MHz, DMSO-d₆) δ 9.44 (s, 1H), 8.90 (d, J=2.8 Hz, 1H), 8.50 (d, J=10.0 Hz, 1H), 8.44 (dd, J=8.8, 2.8 Hz, 1H), 8.00 (d, J=5.7 Hz, 1H), 7.46 (d, J=8.8 Hz, 1H), 7.38-7.36 (m, 2H), 7.19 (d, J=5.8 Hz, 1H), 4.41 (d, J=9.6 Hz, 1H), 4.34 (d, J=9.6 Hz, 1H), 4.10 (d, J=9.2 Hz, 1H), 3.96-3.91 (m, 3H), 2.47-2.41 (m, 1H), 2.26 (m, 1H); MS (ES+) m/z 381.0 (M+1), 383.0 (M+1). Second eluting enantiomer (0.048 g, 16% yield): ¹H NMR (400 MHz, DMSO-d₆) δ 9.43 (s, 1H), 8.89 (d, J=2.6 Hz, 1H), 8.48 (s, 1H), 8.44 (dd, J=8.8, 2.9 Hz, 1H), 7.99 (d, J=5.8 Hz, 1H), 7.46 (d, J=8.7 Hz, 1H), 7.37 (dd, J=6.7, 2.7 Hz, 2H), 7.19 (d, J=5.8 Hz, 1H), 4.41 (d, J=9.6 Hz, 1H), 4.34 (d, J=9.6 Hz, 1H), 4.10 (d, J=9.2 Hz, 1H), 3.96-3.91 (m, 3H), 2.46-2.41 (m, 1H), 2.26 (m, 1H); MS (ES+) m/z 381.0 (M+1), 383.0 (M+1).

### Example 127

### Synthesis of 1-(((1-((2-methylpyrimidin-5-yl)amino)isoquinolin-6-yl)oxy)methyl)cyclopropane-1-carbonitrile

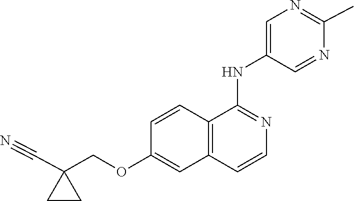 [SMILES: Cc1ncc(Nc2nccc3cc(OCC4(C#N)CC4)ccc23)cn1]

To a mixture of 1-(((1-chloroisoquinolin-6-yl)oxy)methyl)cyclopropane-1-carbonitrile (0.050 g, 0.193 mmol), 2-dicyclohexylphosphino-2′,6′-dimethoxy-1,1′-biphenyl (0.016 g, 0.0387 mmol), 2-methylpyrimidin-5-amine (0.023 mg, 0.213 mmol), and tris(dibenzylideneacetone)dipalladium(0) (0.018 g, 0.0193 mmol) in 1,4-dioxane (5 mL) was added potassium phosphate tribasic (0.164 g, 0.773 mmol) and the mixture was degassed with a stream of nitrogen for 5 minutes. The reaction mixture was then heated at 120° C. for 2 h. After cooling to ambient temperature, the reaction mixture was filtered through celite, and the filtrate was concentrated in vacuo to afford a residue. The residue was purified by silica gel column chromatography, eluting with a gradient of 0 to 100% of ethyl acetate in heptane, to provide the title compound as a colorless solid (0.028 g, 42% yield): ¹H NMR (400 MHz, DMSO-d₆) δ 9.39-9.38 (m, 1H), 9.16 (s, 2H), 8.47 (d, J=9.3 Hz, 1H), 7.95 (d, J=5.8 Hz, 1H), 7.38 (dd, J=9.2, 2.6 Hz, 1H), 7.27 (d, J=2.5 Hz, 1H), 7.15 (d, J=5.8 Hz, 1H), 4.22 (s, 2H), 2.59 (s, 3H), 1.35-1.45 (m, 2H), 1.15-1.25 (m, 2H); MS (ES+) m/z 332.0 (M+1).

### Example 128

### Synthesis of N-(6-chloropyridin-3-yl)-6-((3-fluoro-1-methylazetidin-3-yl)methoxy)isoquinolin-1-amine

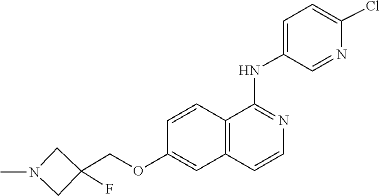 [SMILES: CN1CC(F)(COc2ccc3c(Nc4ccc(Cl)nc4)nccc3c2)C1]

To a solution of N-(6-chloropyridin-3-yl)-6-((3-fluoroazetidin-3-yl)methoxy)isoquinolin-1-amine hydrochloride (0.0750 g, 0.190 mmol) and paraformaldehyde (0.0280 g, 0.933 mmol) in dichloromethane (5 mL) and methanol (2 mL) was added one drop of acetic acid and sodium acetate (0.0310 g, 0.378 mmol). The mixture was stirred at ambient temperature for 1 h, then sodium triacetoxyborohydride (0.125 g, 0.590 mmol) was added to it. The reaction mixture was stirred at ambient temperature for 12 g, and then diluted with ethyl acetate (20 mL). The mixture was washed with brine (3×20 mL), and the organic phase was dried over anhydrous sodium sulfate and filtered. The filtrate was concentrated under reduced pressure.

The residue was purified by reverse-phase preparative HPLC, eluting with a gradient of 24 to 52% of acetonitrile in water containing 0.1% of ammonium hydroxide, to afford the title compound as a white solid (0.0168 g, 23% yield): ¹H NMR (400 MHz, DMSO-d₆) δ9.39 (s, 1H), 8.88 (d, J=2.4 Hz, 1H), 8.49-8.37 (m, 2H), 7.97 (d, J=5.6 Hz, 1H), 7.44 (d, J=8.8 Hz, 1H), 7.38-7.30 (m, 2H), 7.17 (d, J=5.6 Hz, 1H), 4.52-4.38 (m, 2H), 3.60-3.52 (m, 2H), 3.23-3.12 (m, 2H), 2.34 (s, 3H); MS (ES+) m/z 373.1 (M+1), 375.1 (M+1).

### Example 129

### Synthesis of (5-((6-((1-fluorocyclopropyl)methoxy)isoquinolin-1-yl)amino)pyrimidin-2-yl)methanol

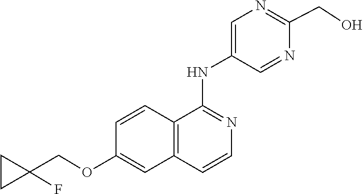 [SMILES: OCc1ncc(Nc2nccc3cc(OCC4(F)CC4)ccc23)cn1]

### Step 1: Preparation of methyl 5-((6-((1-fluorocyclopropyl)methoxy)isoquinolin-1-yl)amino)pyrimidine-2-carboxylate

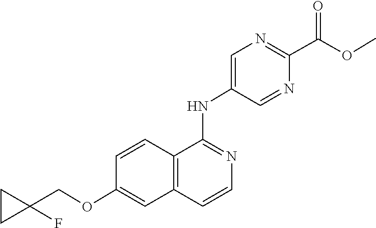 [SMILES: COC(=O)c1ncc(Nc2nccc3cc(OCC4(F)CC4)ccc23)cn1]

To a mixture of 1-chloro-6-((1-fluorocyclopropyl)methoxy)isoquinoline (0.110 g, 0.435 mmol) and methyl 5-aminopyrimidine-2-carboxylate (0.0800 g, 0.522 mmol) in 2-methylbutan-2-ol (10 mL) was added methanesulfonato(2-dicyclohexylphosphino-2′,6′-di-isopropoxy-1,1′-biphenyl)(2′-amino-1,1′-biphenyl-2-yl)palladium(II) ((0.036 g, 0.044 mmol) and cesium carbonate (0.426 g, 1.31 mmol). The reaction mixture was heated to 100° C. for 2.5 h in a microwave reactor. After cooling to ambient temperature, the reaction mixture was diluted with water (20 mL) and extracted with ethyl acetate (3×30 mL). The combined organic layers were washed with brine (3×20 mL), dried over anhydrous sodium sulfate, and filtered. Concentration of the filtrate under reduced pressure and purification of the obtained residue by silica gel column chromatography, eluting with a gradient of 0 to 80% of ethyl acetate in petroleum ether, afforded the title compound as a yellowish solid (0.115 g, 45% yield): ¹H NMR (400 MHz, DMSO-d₆) δ 9.81 (s, 1H), 9.49 (s, 2H), 8.49 (d, J=9.2 Hz, 1H), 8.15-8.02 (m, 1H), 7.42 (dd, J=2.4, 9.2 Hz, 1H), 7.37 (d, J=2.4 Hz, 1H), 7.29 (d, J=5.6 Hz, 1H), 4.52-4.49 (m, 1H), 4.45 (s, 1H), 3.88 (s, 3H), 1.21-1.14 (m, 2H), 0.96-0.90 (m, 2H); MS (ES+) m/z 369.1 (M+1).

### Step 2: Preparation of (5-((6-((1-fluorocyclopropyl)methoxy)isoquinolin-1-yl)amino)pyrimidin-2-yl)methanol

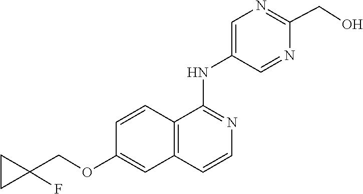 [SMILES: OCc1ncc(Nc2nccc3cc(OCC4(F)CC4)ccc23)cn1]

To a solution of methyl 5-((6-((1-fluorocyclopropyl)methoxy)isoquinolin-1-yl)amino)pyrimidine-2-carboxylate (0.080 g, 0.217 mmol) in tetrahydrofuran (8 mL) was added lithium aluminum hydride (1.0 M in tetrahydrofuran, 0.434 mL) dropwise at 0° C. The mixture was allowed to warm to ambient temperature and stirred for 3 h. To it was then added sodium sulfate decahydrate (0.030 g) and the mixture was stirred at ambient temperature for 30 minutes. The mixture was filtered through a plug of celite, and the filtrate was concentrated under reduced pressure to give a residue. The residue was purified by reverse-phase preparative HPLC, eluting with a gradient of 25 to 55% of acetonitrile in water containing 0.1% of ammonium hydroxide, to provide the title compound as a colorless solid (0.0217 g, 29% yield): ¹H NMR (400 MHz, DMSO-d₆) δ 9.39 (s, 1H), 9.25 (s, 2H), 8.45 (d, J=9.2 Hz, 1H), 7.97 (d, J=5.6 Hz, 1H), 7.37 (dd, J=2.4, 9.2 Hz, 1H), 7.32 (d, J=2.0 Hz, 1H), 7.17 (d, J=5.6 Hz, 1H), 5.20 (t, J=6.0 Hz, 1H), 4.57 (d, J=6.0 Hz, 2H), 4.49 (s, 1H), 4.44 (s, 1H), 1.22-1.14 (m, 2H), 0.95-0.90 (m, 2H); MS (ES+) m/z 369.1 (M+1).

### Examples 130 AND 131

### Synthesis of (R)—N-(6-chloropyridin-3-yl)-6-(1-methoxypropyl)isoquinolin-1-amine and (S)—N-(6-chloropyridin-3-yl)-6-(1-methoxypropyl)isoquinolin-1-amine

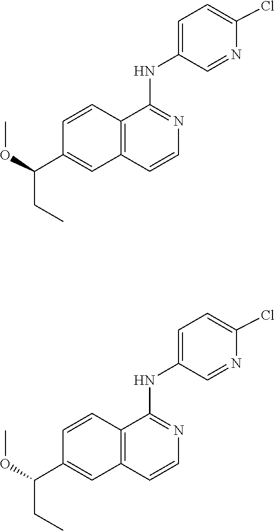 [SMILES: CC[C@@H](OC)c1ccc2c(Nc3ccc(Cl)nc3)nccc2c1 ; CC[C@H](OC)c1ccc2c(Nc3ccc(Cl)nc3)nccc2c1]

### Step 1: Preparation of 1-(1-chloroisoquinolin-6-yl)propan-1-one

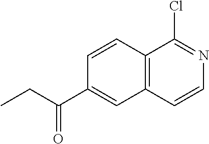

To a solution of 6-bromo-1-chloro-isoquinoline (5.00 g, 20.6 mmol) in tetrahydrofuran (50 mL) was added n-butyllithium (2.50 M in hexane, 9.90 mL) dropwise at −60° C. The reaction mixture was stirred at this temperature for 30 minutes, and then N-methoxy-N-methyl-propanamide (2.90 g, 24.7 mmol) was added dropwise to it. The resulting mixture was stirred at −60° C. for 1 h. The reaction mixture was poured into ice water (200 mL), and the aqueous layer was extracted with dichloromethane (3×200 mL). The combined organic layers were washed with brine (50 mL), dried over anhydrous sodium sulfate, and filtered. Concentration of the filtrate under reduced pressure and purification of the residue by silica gel column chromatography, eluting with a gradient of 10 to 25% of ethyl acetate in petroleum ether, provided the title compound as a colorless solid (2.00 g, 40% yield): ¹H NMR (400 MHz, CDCl₃) δ 8.44-8.41 (m, 2H), 8.37 (d, J=5.6 Hz, 1H), 8.22 (dd, J=8.8, 1.6 Hz, 1H), 7.73 (d, J=5.6 Hz, 1H), 3.17 (q, J=7.2 Hz, 2H), 1.31 (t, J=7.2 Hz, 3H).

### Step 2: Preparation of 1-(1-chloroisoquinolin-6-yl)propan-1-ol

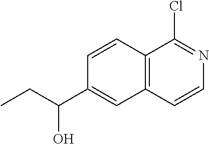

To a solution of 1-(1-chloro-6-isoquinolyl)propan-1-one (1.00 g, 4.55 mmol) in tetrahydrofuran (30 mL) was slowly added (S)-1-methyl-3,3-diphenyl-3a,4,5,6-tetrahydropyrrolo[1,2-c][1,3,2]oxazaborole (1.0 M, 1.78 mL), followed by borane tetrahydrofuran complex (1.0 M, 5.01 mL). The reaction mixture was stirred at ambient temperature for 16 h. The reaction mixture was cooled to 0° C. and methanol (3 mL) was added to it. After stirring for 20 minutes, the reaction mixture was concentrated in vacuo. The obtained residue was dissolved in dichloromethane (40 mL) and washed with brine (30 mL). The organic phase was dried over sodium sulfate, filtered, and the filtrate concentrated in vacuo. The residue was purified by silica gel column chromatography, eluting with a gradient of 33 to 48% of ethyl acetate in petroleum ether, to provide the title compound as a colorless liquid (1.00 g, 99% yield): ¹H NMR (400 MHz, CDCl₃) δ 8.33-8.26 (m, 2H), 7.82 (s, 1H), 7.68 (dd, J=8.8, 1.6 Hz, 1H), 7.61-7.58 (m, 1H), 4.86 (t, J=6.4 Hz, 1H), 1.93-1.84 (m, 2H), 0.97 (t, J=7.2 Hz, 3H).

### Step 3: Preparation of 1-chloro-6-(1-methoxypropyl)isoquinoline

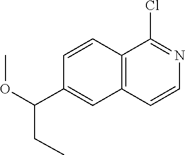 [SMILES: CCC(OC)c1ccc2c(Cl)nccc2c1]

To a mixture of sodium hydride (60% dispersion in mineral oil, 0.271 g, 6.77 mmol) in tetrahydrofuran (20 mL) was added a solution of 1-(1-chloro-6-isoquinolyl)propan-1-ol (1.00 g, 4.51 mmol) in tetrahydrofuran (20 mL) and the mixture was stirred for 1 h. To it was then added iodomethane (2.56 g, 18.0 mmol) and the resulting mixture was stirred at ambient temperature for 16 h. The reaction mixture was poured into water (100 mL). The aqueous phase was extracted with ethyl acetate (3×30 mL). The combined organic phase was washed with brine (30 mL), dried with anhydrous sodium sulfate, filtered and the filtrate concentrated under reduced pressure. The obtained residue was purified by silica gel column chromatography, eluting with a gradient of 5 to 10% of ethyl acetate in petroleum ether, to provide the title compound as yellow oil (0.900 g, 85% yield): ¹H NMR (400 MHz, CDCl₃) δ 8.35 (d, J=2.8 Hz, 1H), 8.28 (dd, J=8.8, 2.8 Hz, 1H), 7.74 (s, 1H), 7.66-7.63 (m, 1H), 7.60 (d, J=8.8 Hz, 1H), 4.25 (t, J=6.4 Hz, 1H), 3.28 (s, 3H), 1.93-1.86 (m, 1H), 1.80-1.74 (m, 1H), 0.92 (t, J=7.2 Hz, 3H).

### Step 4: Preparation of N-(6-chloropyridin-3-yl)-6-(1-methoxypropyl)isoquinolin-1-amine

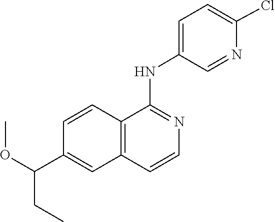 [SMILES: CCC(OC)c1ccc2c(Nc3ccc(Cl)nc3)nccc2c1]

To a solution of 1-chloro-6-(1-methoxypropyl)isoquinoline (0.800 g, 3.39 mmol), 6-chloropyridin-3-amine (0.524 g, 4.07 mmol), and cesium carbonate (2.76 g, 8.49 mmol) in 2-methylbutan-2-ol (20 mL) was added [(2-di-tert-butylphosphino-2′,4′,6′-triisopropyl-1,1′-biphenyl)-2-(2′-amino-1,1′-biphenyl)]palladium(II) methanesulfonate (0.404 g, 0.509 mmol). The reaction mixture was heated to 100° C. for 16 h under nitrogen. After cooling to ambient temperature, the reaction mixture was poured into ice-water (30 mL). The aqueous layer was extracted with ethyl acetate (3×30 mL). The combined organic layers were washed with brine (30 mL), dried over anhydrous sodium sulfate, filtered, and the filtrate concentrated in vacuo. The obtained residue was purified by silica gel column chromatography, eluting with a gradient of 10 to 33% of ethyl acetate in petroleum ether, to provide the title compound as a yellow solid (0.310 g, 26% yield): ¹H NMR (400 MHz, CDCl₃) δ 8.55 (d, J=2.8 Hz, 1H), 8.40 (dd, J=8.4, 2.8 Hz, 1H), 8.09 (d, J=5.6 Hz, 1H), 7.97 (d, J=8.8 Hz, 1H), 7.67 (d, J=0.8 Hz, 1H), 7.57 (d, J=1.6 Hz, 1H), 7.33 (d, J=8.4 Hz, 1H), 7.21 (d, J=5.6 Hz, 1H), 4.22 (t, J=6.4 Hz, 1H), 3.28 (s, 3H), 2.00-1.92 (m, 1H), 1.82-1.72 (m, 1H), 0.91 (t, J=7.2 Hz, 3H), NH not observed.

### Step 5: Preparation of (R) and (S)—N-(6-chloropyridin-3-yl)-6-(1-methoxypropyl)isoquinolin-1-amine

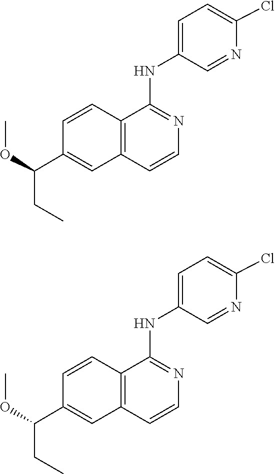 [SMILES: CC[C@@H](OC)c1ccc2c(Nc3ccc(Cl)nc3)nccc2c1 ; CC[C@H](OC)c1ccc2c(Nc3ccc(Cl)nc3)nccc2c1]

Racemic N-(6-chloropyridin-3-yl)-6-(1-methoxypropyl)isoquinolin-1-amine (0.400 g, 1.22 mmol) was purified by chiral SFC using a Daicel Chiralpak AD column (250×30 mm, 10 μM), eluting with of 45% methanol containing 0.1% of ammonium hydroxide in supercritical carbon dioxide, to afford the title compounds as single enantiomers as yellowish solids. First eluting enantiomer (0.206 g, 51% yield): ¹H NMR (400 MHz, DMSO-d₆) δ 9.47 (s, 1H), 8.90 (d, J=2.8 Hz, 1H), 8.53 (d, J=8.8 Hz, 1H), 8.44 (dd, J=8.8, 2.8 Hz, 1H), 8.03 (d, J=5.6 Hz, 1H), 7.75 (s, 1H), 7.59 (d, J=8.4 Hz, 1H), 7.47 (d, J=8.4 Hz, 1H), 7.29 (d, J=5.6 Hz, 1H), 4.29 (t, J=6.4 Hz, 1H), 3.19 (s, 3H), 1.89-1.62 (m, 2H), 0.84 (t, J=7.2 Hz, 3H); MS (ES+) m/z 328.1 (M+1), 330.1 (M+1). Second eluting enantiomer (0.0760 g, 19% yield): ¹H NMR (400 MHz, DMSO-d₆) δ 9.47 (s, 1H), 8.90 (d, J=2.8 Hz, 1H), 8.53 (d, J=8.8 Hz, 1H), 8.44 (dd, J=8.8, 2.8 Hz, 1H), 8.03 (d, J=5.6 Hz, 1H), 7.75 (s, 1H), 7.59 (d, J=8.4 Hz, 1H), 7.47 (d, J=8.4 Hz, 1H), 7.29 (d, J=5.6 Hz, 1H), 4.29 (t, J=6.4 Hz, 1H), 3.19 (s, 3H), 1.89-1.62 (m, 2H), 0.84 (t, J=7.2 Hz, 3H); MS (ES+) m/z 328.1, 330.1 (M+1).

### Examples 132 and 133

In a similar manner as described in EXAMPLES 130 and 131, utilizing the appropriately substituted starting materials and intermediates, the following compounds were prepared:

| | | MS | |
|---|---|---|---|
| | | (ES+) | |
| Example No | Name | m/z | NMR |
| 132 | (R)-N-(6-chloropyridin- | 314.0 | ¹H NMR (400 MHz, DMSO-d₆) δ 9.46 |
| | 3-yl)-6-(1- | (M + 1), | (s, 1H), 8.90 (d, J = 2.8 Hz, 1H), 8.52 |
| | methoxyethyl)isoquinolin- | 316.0 | (d, J = 8.8 Hz, 1H), 8.44 (dd, J = 2.8, |
| | 1-amine | (M + 1). | 8.8 Hz, 1H), 8.03 (d, J = 5.6 Hz, 1H), |
| | | | 7.77 (d, J = 1.2 Hz, 1H), 7.61 (dd, J = |
| | | | 1.6, 8.8 Hz, 1H), 7.46 (d, J = 8.8 Hz, |
| | | | 1H), 7.28 (d, J = 5.6 Hz, 1H), 4.52 (q, |
| | | | J = 6.4 Hz, 1H), 3.19 (s, 3H), 1.42 (d, |
| | | | J = 6.5 Hz, 3H). |
| 133 | (S)-N-(6-chloropyridin- | 314.0 | ¹H NMR (400 MHz, DMSO-d₆) δ 9.78 |
| | 3-yl)-6-(1- | (M + 1), | (s, 1H), 8.86 (d, J = 2.8 Hz, 1H), 8.56 |
| | methoxyethyl)isoquinolin- | 316.0 | (d, J = 8.8 Hz, 1H), 8.38 (d, J = 8.4 |
| | 1-amine | (M + 1). | Hz, 1H), 7.96 (d, J = 6.0 Hz, 1H), 7.81 |
| | | | (s, 1H), 7.65 (d, J = 9.2 Hz, 1H), 7.51 |
| | | | (d, J = 8.8 Hz, 1H), 7.31 (d, J = 6.0 |
| | | | Hz, 1H), 4.54 (q, J = 6.4 Hz, 1H), 3.20 |
| | | | (s, 3H), 1.43 (d, J = 6.4 Hz, 3H). |

### Example 134

### Synthesis of 2-(3-(((1-((6-chloropyridin-3-yl)amino)isoquinolin-6-yl)oxy)methyl)oxetan-3-yl)acetonitrile

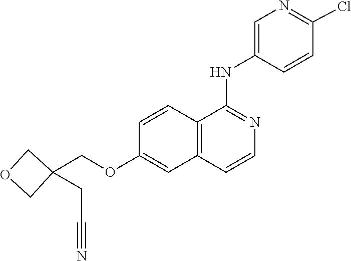 [SMILES: N#CCC1(COc2ccc3c(Nc4ccc(Cl)nc4)nccc3c2)COC1]

### Step 1. Preparation of 2-(3-(((1-((6-chloropyridin-3-yl)((2-(trimethylsilyl)ethoxy)methyl)amino)isoquinolin-6-yl)oxy)methyl)oxetan-3-yl)acetonitrile

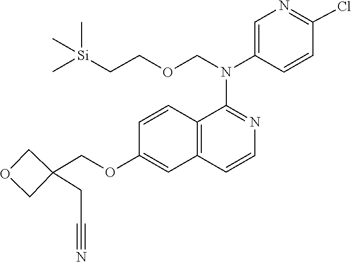 [SMILES: C[Si](C)(C)CCOCN(c1ccc(Cl)nc1)c1nccc2cc(OCC3(CC#N)COC3)ccc12]

To a solution of 2-(3-(hydroxymethyl)oxetan-3-yl)acetonitrile (0.0480 g, 0.378 mmol) in tetrahydrofuran (6 mL) was added sodium hydride (60% dispersion in mineral oil, 0.0160 g, 0.400 mmol) at 0° C. and the resulting mixture was stirred for 30 minutes at this temperature. To the reaction mixture was then added a solution of N-(6-chloropyridin-3-yl)-6-fluoro-N-((2-(trimethylsilyl)ethoxy)methyl)isoquinolin-1-amine (0.050 g, 0.124 mmol) in tetrahydrofuran at 0° C. The reaction mixture was allowed to warm to ambient temperature and then heated to 60° C. for 16 h. After cooling to ambient temperature, the mixture was quenched by addition of saturated ammonium chloride solution (20 mL) and extracted by ethyl acetate (3×5 mL). The combined organic phase was washed with brine (3×5 mL), dried over anhydrous sodium sulfate, and filtered. Concentration of the filtrate in vacuo and purification of the obtained residue by silica gel column chromatography, eluting with a gradient of 0 to 60% of ethyl acetate in petroleum ether, provided the title compounds as a yellowish gum (0.0640 g, 84% yield): ¹H NMR (400 MHz, DMSO-d₆) δ 8.32 (d, J=5.6 Hz, 1H), 7.91 (d, J=2.8 Hz, 1H), 7.69 (s, 1H), 7.67 (d, J=4.8 Hz, 1H), 7.54 (d, J=2.4 Hz, 1H), 7.31 (d, J=8.8 Hz, 1H), 7.22 (dd, J=9.2, 2.4 Hz, 1H), 7.15 (dd, J=8.8, 3.2 Hz, 1H), 5.36 (s, 2H), 4.54 (q, J=6.4 Hz, 4H), 4.39 (s, 2H), 3.54 (t, J=8.0 Hz, 2H), 3.11 (s, 2H), 0.79 (t, J=8.0 Hz, 2H), 0.14 (s, 9H).

### Step 2. Preparation of 2-(3-(((1-((6-chloropyridin-3-yl)amino)isoquinolin-6-yl)oxy)methyl)oxetan-3-yl)acetonitrile

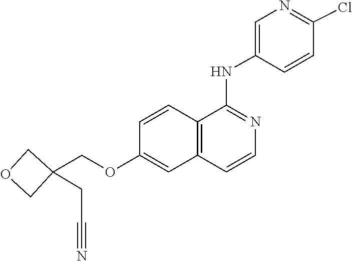 [SMILES: N#CCC1(COc2ccc3c(Nc4ccc(Cl)nc4)nccc3c2)COC1]

To a solution of 2-(3-(((1-((6-chloropyridin-3-yl)((2-(trimethylsilyl)ethoxy)methyl)amino)isoquinolin-6-yl)oxy)methyl)oxetan-3-yl)acetonitrile (0.0600 g, 0.0974 mmol) in dichloromethane (4 mL) was added trifluoroacetic acid (1 mL) and the mixture was stirred at ambient temperature for 16 h. Concentration of the mixture in vacuo and purification of the obtained residue by reserve-phase preparative HPLC, eluting with a gradient of 36 to 66% of acetonitrile in water containing 0.1% ammonium hydroxide, afforded the title compound as an off-white solid (0.0225 g, 60% yield): ¹H NMR ((400 MHz, DMSO-d₆) δ 9.40 (s, 1H), 8.89 (d, J=2.4 Hz, 1H), 8.47 (d, J=9.2 Hz, 1H), 8.43 (dd, J=8.8, 2.4 Hz, 1H), 7.98 (d, J=5.6 Hz, 1H), 7.45 (d, J=8.8 Hz, 1H), 7.38 (d, J=1.6 Hz, 1H), 7.32 (d, J=9.2 Hz, 1H), 7.20 (d, J=5.6 Hz, 1H), 4.60-4.56 (m, 2H), 4.55-4.50 (m, 2H), 4.39 (s, 2H), 3.13 (s, 2H); MS (ES+) m/z 381.2 (M+1), 383.2 (M+1).

### Examples 135-143

In a similar manner as described in EXAMPLE 134, utilizing the appropriately substituted starting materials and intermediates, the following compounds were prepared:

| | | MS | |
|---|---|---|---|
| | | (ES+) | |
| Example No | Name | m/z | NMR |
| 135 | (S)-6-((2- | 382.2 | ¹H NMR (400 MHz, CD₃OD) δ 8.71 |
| | oxaspiro[3.4]octan-6- | (M + 1), | (d, J = 2.8 Hz, 1H), 8.24 (dt, J = 8.8, |
| | yl)oxy)-N-(6- | 384.2 | 4.4 Hz, 2H), 7.89 (d, J = 5.9 Hz, 1H), |
| | chloropyridin-3- | (M + 1) | 7.38 (d, J = 8.7 Hz, 1H), 7.16-7.11 (m, |
| | yl)isoquinolin-1-amine | | 3H), 5.02-4.98 (m, 1H), 4.69 (t, J = |
| | | | 6.1 Hz, 2H), 4.62 (dd, J = 5.8, 3.2 Hz, |
| | | | 2H), 2.31-2.21 (m, 3H), 2.18-2.10 (m, |
| | | | 1H), 2.07-2.01 (m, 1H), 1.97-1.90 (m, |
| | | | 1H), NH not observed. |
| 136 | N-(6-chloropyridin-3-yl)- | 352.1 | ¹H NMR (400 MHz, DMSO-d₆) δ 9.38 |
| | 6-((2,2-dimethylbut-3- | (M + 1), | (s, 1H), 8.89 (d, J = 2.8 Hz, 1H), |
| | yn-1-yl)oxy)isoquinolin- | 354.1 | 8.51-8.38 (m, 2H), 7.96 (d, J = 5.6 |
| | 1-amine | (M + 1). | Hz, 1H), 7.51-7.39 (m, 1H), 7.37- |
| | | | 7.28 (m, 2H), 7.19 (d, J = 6.0 Hz, 1H), |
| | | | 4.01 (s, 2H), 3.03 (s, 1H), 1.33 (s, |
| | | | 6H). |
| 137 | 6-([1,1′- | 366.1 | ¹H NMR (400 MHz, DMSO-d₆) δ 9.36 |
| | bi(cyclopropan)]-1- | (M + 1), | (s, 1H), 8.88 (d, J = 2.8 Hz, 1H), |
| | ylmethoxy)-N-(6- | 368.0 | 8.45-8.40 (m, 2H), 7.95 (d, J = 5.6 |
| | chloropyridin-3- | (M + 1). | Hz, 1H), 7.44 (d, J = 8.8 Hz, 1H), |
| | yl)isoquinolin-1-amine | | 7.31-7.25 (m, 2H), 7.16 (d, J = 5.6 |
| | | | Hz, 1H), 4.03 (s, 2H), 1.35-1.28 (m, |
| | | | 1H), 0.50-0.47 (m, 2H), 0.39-0.32 |
| | | | (m, 4H), 0.13-0.07 (m, 2H). |
| 138 | (R)-6-((2- | 382.2 | ¹H NMR (400 MHz, CD₃OD) δ 8.71 |
| | oxaspiro[3.4]octan-6- | (M + 1), | (d, J = 2.8 Hz, 1H), 8.25-8.21 (m, 2H), |
| | yl)oxy)-N-(6- | 384.2 | 7.88 (d, J = 5.9 Hz, 1H), 7.38 (d, J = |
| | chloropyridin-3- | (M + 1). | 8.7 Hz, 1H), 7.15-7.11 (m, 3H), 4.99 |
| | yl)isoquinolin-1-amine | | (tt, J = 5.2, 2.6 Hz, 1H), 4.69 (t, J = |
| | | | 6.1 Hz, 2H), 4.62 (dd, J = 5.8, 3.2 Hz, |
| | | | 2H), 2.30-2.21 (m, 3H), 2.18-2.00 (m, |
| | | | 2H), 1.96-1.89 (m, 1H), NH not |
| | | | observed. |
| 139 | (R)-N-(6-chloropyridin- | 358.2 | ¹H NMR (400 MHz, CD₃OD) δ 8.74 (d, |
| | 3-yl)-6-(1-(1- | (M + 1), | J = 2.8 Hz, 1H), 8.28 (td, J = 6.0, 3.3 |
| | fluorocyclopropyl)ethoxy)isoquinolin- | 360.2 | Hz, 2H), 7.91 (d, J = 5.9 Hz, 1H), 7.41 |
| | 1-amine | (M + 1). | (d, J = 8.7 Hz, 1H), 7.27-7.25 (m, 2H), |
| | | | 7.17 (d, J = 5.9 Hz, 1H), 4.74 (dq, J = |
| | | | 18.7, 6.3 Hz, 1H), 1.52 (d, J = 6.3 Hz, |
| | | | 3H), 1.17-1.03 (m, 2H), 0.92-0.82 (m, |
| | | | 2H), NH not observed. |
| 140 | (S)-N-(6-chloropyridin- | 358.2 | ¹H NMR (400 MHz, CD₃OD) δ 8.72 |
| | 3-yl)-6-(1-(1- | (M + 1), | (d, J = 2.7 Hz, 1H), 8.28-8.24 (m, 2H), |
| | fluorocyclopropyl)ethoxy)isoquinolin- | 360.2 | 7.89 (d, J = 5.9 Hz, 1H), 7.39 (d, J = |
| | 1-amine | (M + 1). | 8.7 Hz, 1H), 7.24 (dd, J = 7.1, 2.7 Hz, |
| | | | 2H), 7.15 (d, J = 5.9 Hz, 1H), 4.76- |
| | | | 4.67 (m, 1H), 1.51-1.48 (m, 3H), 1.15- |
| | | | 1.01 (m, 2H), 0.90-0.81 (m, 2H), NH |
| | | | not observed. |
| 141 | N-(6-chloropyridin-3-yl)- | 358.2 | ¹H NMR (400 MHz, DMSO-d₆) |
| | 6-((1- | (M + 1), | δ 10.12 (s, 1H), 8.79 (d, J = 2.6 Hz, |
| | fluorocyclobutyl)methoxy)isoquinolin- | 360.2 | 1H), 8.53 (d, J = 9.1 Hz, 1H), 8.30- |
| | 1-amine | (M + 1). | 8.27 (m, 1H), 7.84-7.82 (m, 1H), 7.58 |
| | | | (d, J = 8.6 Hz, 1H), 7.47-7.43 (m, 2H), |
| | | | 7.24 (d, J = 6.2 Hz, 1H), 4.35-4.45 (m, |
| | | | 2H), 2.39-2.29 (m, 4H), 1.87-1.79 (m, |
| | | | 1H), 1.69-1.61 (m, 1H). |
| 142 | cis-2-((1-((6- | 370.2 | ¹H NMR (400 MHz, DMSO-d₆) δ 9.58 |
| | chloropyridin-3- | (M + 1), | (s, 1H), 8.84 (d, J = 2.7 Hz, 1H), 8.44 |
| | yl)amino)isoquinolin-6- | 372.2 | (d, J = 9.2 Hz, 1H), 8.39-8.36 (m, 1H), |
| | yl)oxy)cyclohexan-1-ol | (M + 1). | 7.90-7.88 (m, 1H), 7.51-7.49 (m, 1H), |
| | | | 7.38-7.38 (m, 1H), 7.33-7.30 (m, 1H), |
| | | | 7.20-7.19 (m, 1H), 4.99 (s, 1H), 4.34- |
| | | | 4.28 (m, 1H), 3.62-3.56 (m, 1H), 2.12- |
| | | | 2.07 (m, 1H), 1.93-1.88 (m, 1H), 1.68- |
| | | | 1.63 (m, 2H), 1.42-1.23 (m, 4H). |
| 143 | trans-2-((1-((6- | 370.2 | ¹H NMR (400 MHz, DMSO-d₆) δ 9.36 |
| | chloropyridin-3- | (M + 1), | (s, 1H), 8.88 (d, J = 2.8 Hz, 1H), 8.43 |
| | yl)amino)isoquinolin-6- | 372.2 | (td, J = 5.8, 3.1 Hz, 2H), 7.93 (d, J = |
| | yl)oxy)cyclohexan-1-ol | (M + 1). | 5.8 Hz, 1H), 7.44 (d, J = 8.7 Hz, 1H), |
| | | | 7.32-7.27 (m, 2H), 7.16 (d, J = 5.8 Hz, |
| | | | 1H), 4.71 (s, 1H), 4.62-4.59 (m, 1H), |
| | | | 3.89-3.87 (m, 1H), 1.98-1.90 (m, 1H), |
| | | | 1.80-1.73 (m, 1H), 1.67-1.51 (m, 4H), |
| | | | 1.41-1.31 (m, 2H). |

### Example 144

### Synthesis of N-(2-chloropyrimidin-5-yl)-6-((1-fluorocyclopropyl)methoxy)-N-methylisoquinolin-1-amine

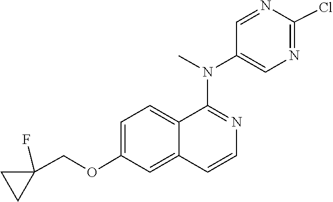 [SMILES: CN(c1cnc(Cl)nc1)c1nccc2cc(OCC3(F)CC3)ccc12]

To a solution of N-(2-chloropyrimidin-5-yl)-6-((1-fluorocyclopropyl)methoxy)isoquinolin-1-amine (0.050 g, 0.145 mmol) in N,N-dimethylformamide (5 mL) was added sodium hydride (60 dispersion in mineral oil, 0.017 g, 0.435 mmol) at 0° C. and the resulting mixture was stirred for 20 minutes at this temperature. To the reaction mixture was then added iodomethane (0.027 mL, 0.435 mmol) at 0° C. The reaction mixture was allowed to warm to ambient temperature and stirred for 16 h. The mixture was then diluted with ethyl acetate (20 mL) and washed with saturated aqueous ammonium chloride (20 mL) and brine (20 mL). The organic phase was dried over anhydrous sodium sulfate, filtered, and the filtrate was concentrated in vacuo. The obtained residue was purified by silica gel column chromatography, eluting with a gradient of 0 to 60% of ethyl acetate in heptane, to provide the title compound as a colorless solid (0.0357 g, 68% yield): ¹H NMR (400 MHz, DMSO-d₆) δ 8.26 (d, J=5.7 Hz, 1H), 8.24 (s, 2H), 7.83 (d, J=9.3 Hz, 1H), 7.64 (d, J=5.5 Hz, 1H), 7.48 (d, J=2.5 Hz, 1H), 7.32 (dd, J=9.2, 2.6 Hz, 1H), 4.48 (d, J=22.6 Hz, 2H), 3.47 (s, 3H), 1.17 (dt, J=18.7, 6.9 Hz, 2H), 0.95-0.89 (m, 2H); MS (ES+) m/z 359.2 (M+1), 361.2 (M+1).

### Example 145

### Synthesis of rac-(1R,3S)-3-((1-((2-chloropyrimidin-5-yl)amino)isoquinolin-6-yl)oxy)-1-(trifluoromethyl)cyclohexan-1-ol

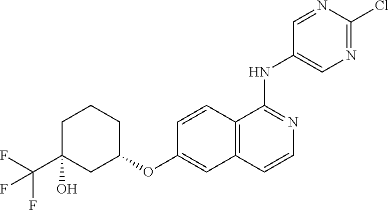 [SMILES: O[C@]1(C(F)(F)F)CCC[C@H](Oc2ccc3c(Nc4cnc(Cl)nc4)nccc3c2)C1]

### Step 1. Preparation of 1-(trifluoromethyl)cyclohexane-1,3-diol

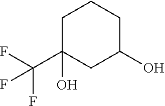 [SMILES: OC1CCCC(O)(C(F)(F)F)C1]

To a solution of 3-hydroxycyclohexan-1-one (0.100 g, 0.876 mmol) in tetrahydrofuran (2 mL) was added trimethyl(trifluoromethyl)silane (0.65 mL, 4.38 mmol) at −40° C. To the mixture was then slowly added a 1.0 M solution of tetrabutylammonium fluoride (0.88 mL, 0.876 mmol) in tetrahydrofuran. The reaction mixture was allowed to warm to ambient temperature and stirred for 16 h. The mixture was poured into saturated ammonium chloride (30 mL) and extracted with dichloromethane (2×20 mL). The combined organic phase was washed with brine (40 mL), dried over anhydrous sodium sulfate, and filtered. The filtrate was concentrated in vacuo to give the title compound as colorless oil (0.185 g, 92% yield): ¹H NMR (400 MHz, DMSO-d₆) δ 5.81 (s, 1H), 5.03 (d, J=4.5 Hz, 1H), 3.94-3.91 (m, 1H), 1.81-1.74 (m, 2H), 1.68-1.51 (m, 4H), 1.31 (q, J=7.3 Hz, 2H).

### Step 2. Preparation of 3-((1-chloroisoquinolin-6-yl)oxy)-1-(trifluoromethyl)cyclohexan-1-ol

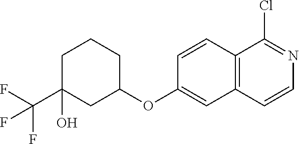 [SMILES: OC1(C(F)(F)F)CCCC(Oc2ccc3c(Cl)nccc3c2)C1]

To a solution of 1-(trifluoromethyl)cyclohexane-1,3-diol (0.093 g, 0.405 mmol) in N,N-dimethylformamide (4 mL) was added sodium hydride (60% dispersion in mineral oil, 0.062 g, 1.54 mmol). The reaction mixture was stirred at ambient temperature for 5 minutes, and to it was then added 1-chloro-6-fluoroisoquinoline (0.070 g, 0.385 mmol). The reaction mixture was stirred at ambient temperature for 4 h. The mixture was diluted with ethyl acetate (50 mL) and washed with saturated aqueous sodium bicarbonate (50 mL) and brine (50 mL). The organic phase was dried over anhydrous sodium sulfate and filtered. The filtrate was concentrated in vacuo to give a residue, which was purified by silica gel column chromatography, eluting with a gradient of 0 to 80% of ethyl acetate in heptane, to afford the title compound as a colorless solid (0.100 g, 75% yield): ¹H NMR (400 MHz, DMSO-d₆) δ 8.18 (t, J=7.5 Hz, 2H), 7.76 (d, J=5.7 Hz, 1H), 7.47 (d, J=2.4 Hz, 1H), 7.40 (dd, J=9.2, 2.5 Hz, 1H), 5.71 (s, 1H), 4.93-4.89 (m, 1H), 2.11-1.94 (m, 3H), 1.88-1.67 (m, 4H), 1.57-1.49 (m, 1H); MS (ES+) m/z 346.4 (M+1), 348.4 (M+1).

### Step 3. Preparation of rac-(1R,3S)-3-((1-((2-chloropyrimidin-5-yl)amino)isoquinolin-6-yl)oxy)-1-(trifluoromethyl)cyclohexan-1-ol

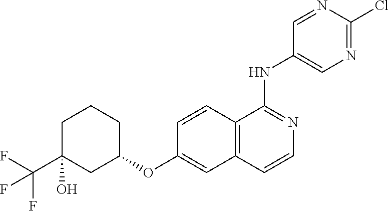 [SMILES: O[C@]1(C(F)(F)F)CCC[C@H](Oc2ccc3c(Nc4cnc(Cl)nc4)nccc3c2)C1]

To a solution of 3-((1-chloroisoquinolin-6-yl)oxy)-1-(trifluoromethyl)cyclohexan-1-ol (0.100 g, 0.289 mmol) in 1,4-dioxane (4 mL) was added was added 5-amino-2-chloropyrimidine (0.034 g, 0.260 mmol), 2-dicyclohexylphosphino-2′,6′-dimethoxy-1,1′-biphenyl (0.018 g, 0.0.043 mmol), tris(dibenzylideneacetone)dipalladium(0) (0.020 g, 0.022 mmol), and potassium phosphate tribasic (0.092 g, 0.434 mmol). The reaction mixture was degassed by passing a stream of nitrogen through it for 5 minutes and then heated to 110° C. for 2 h. After cooling to ambient temperature, the reaction mixture was filtered through celite, and the filtrate was concentrated in vacuo. The obtained residue was purified by silica gel column chromatography, eluting with a gradient of 0 to 100% of ethyl acetate in heptane, to provide the title compound as a colorless solid ((0.016 g, 13% yield)). The relative configuration of the title compound was determined by NOESY NMR. ¹H NMR (400 MHz, DMSO-d₆) δ9.57 (s, 1H), 9.30 (s, 2H), 8.42 (d, J=10.0 Hz, 1H), 7.99 (d, J=5.8 Hz, 1H), 7.31-7.23 (m, 3H), 5.71 (s, 1H), 4.90-4.86 (m, 1H), 2.04-2.01 (m, 2H), 2.00-1.91 (m, 1H), 1.87-1.79 (m, 2H), 1.75-1.72 (m, 2H), 1.56-1.50 (m, 1H); ¹⁹F NMR (376 MHz, DMSO-d₆) δ −81.5 (s); MS (ES+) m/z 439.0 (M+1), 441.0 (M+1).

### Example 146

### Synthesis of 3-((1-((2-chloropyrimidin-5-yl)amino)isoquinolin-6-yl)oxy)-2-cyclopropyl-2-fluoropropanenitrile

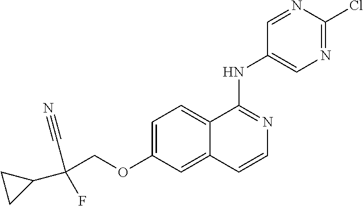 [SMILES: N#CC(F)(COc1ccc2c(Nc3cnc(Cl)nc3)nccc2c1)C1CC1]

### Step 1. Preparation of 2-cyclopropyl-2-fluoro-3-hydroxypropanenitrile

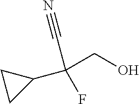

To a mixture of ethyl 2-cyano-2-cyclopropylacetate (0.255 g, 1.58 mmol) in N,N-dimethylformamide (4.5 mL) was added potassium carbonate (0.267 g, 1.93 mmol) and N-fluorobenzenesulfonimide and the reaction mixture was stirred at ambient temperature for 48 h. The reaction mixture was filtered, and the filtrate was poured into water (5 mL). The mixture was extracted with diethyl ether (3×15 mL). The combined organic phase was washed with brine (20 mL), dried over anhydrous sodium sulfate, and filtered. The filtrate was concentrated in vacuo to provide a yellowish oil. To the obtained residue was added methanol (7.5 mL) followed by sodium borohydride (0.150 g, 3.95 mmol). The reaction mixture was stirred at ambient temperature for 2h. The reaction mixture was then concentrated in vacuo. To the obtained residue was added dichloromethane (25 mL). The organic phase was washed with saturated ammonium chloride solution (15 mL), brine (15 mL), and dried over anhydrous sodium sulfate. Concentration of the filtrate in vacuo provided the title compound as a yellowish oil (0.200 g, 98% yield): ¹H NMR (400 MHz, DMSO-d₆) δ 5.95 (t, J=6.2 Hz, 1H), 3.84-3.82 (m, 1H), 3.78 (dd, J=9.1, 6.2 Hz, 1H), 1.48 (dtt, J=10.9, 8.2, 5.3 Hz, 1H), 0.77-0.67 (m, 2H), 0.63-0.58 (m, 1H), 0.56-0.51 (m, 1H); ¹⁹F NMR (376 MHz, DMSO-d₆) δ−157.0 (s).

### Step 2. Preparation of 3-((1-chloroisoquinolin-6-yl)oxy)-2-cyclopropyl-2-fluoropropanenitrile

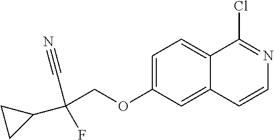 [SMILES: N#CC(F)(COc1ccc2c(Cl)nccc2c1)C1CC1]

Following the procedure as described for EXAMPLE 208, Step 1, and making variations as required to replace 3-(hydroxymethyl)oxetane-3-carbonitrile with 2-cyclopropyl-2-fluoro-3-hydroxypropanenitrile, the title compound was obtained as a colorless solid (0.148 g, 21% yield): MS (ES+) m/z 291.6 (M+1), 293.6 (M+1).

### Step 3. Preparation of 3-(((1-((2-chloropyrimidin-5-yl)amino)isoquinolin-6-yl)oxy)methyl)oxetane-3-carbonitrile

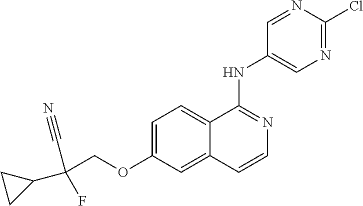 [SMILES: N#CC(F)(COc1ccc2c(Nc3cnc(Cl)nc3)nccc2c1)C1CC1]

Following the procedure as described for EXAMPLE 208, Step 2 and making variations as required to replace 3-(((1-chloroisoquinolin-6-yl)oxy)methyl)oxetane-3-carbonitrile with 3-((1-chloroisoquinolin-6-yl)oxy)-2-cyclopropyl-2-fluoropropanenitrile, the title compound was obtained as a colorless solid (0.021 g, 11% yield): ¹H NMR (400 MHz, DMSO-d₆) δ9.63 (s, 1H), 9.30 (s, 2H), 8.49 (d, J=9.1 Hz, 1H), 8.03 (d, J=5.8 Hz, 1H), 7.47-7.43 (m, 2H), 7.26 (d, J=5.7 Hz, 1H), 4.80 (q, J=9.5 Hz, 1H), 4.75 (s, 1H), 1.77-1.68 (m, 1H), 0.87-0.80 (m, 2H), 0.80-0.75 (m, 1H), 0.74-0.69 (m, 1H); ¹⁹F NMR (376 MHz, DMSO-d₆) δ−155.5 (s); MS (ES+) m/z 384.0 (M+1), 386.0 (M+1).

### Example 147

### Synthesis of 2-(1-(((1-((2-chloropyrimidin-5-yl)amino)isoquinolin-6-yl)oxy)methyl)cyclopropyl)acetonitrile

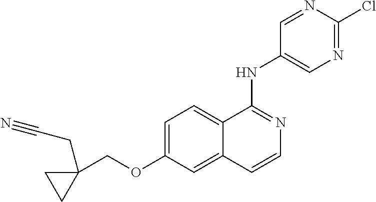 [SMILES: N#CCC1(COc2ccc3c(Nc4cnc(Cl)nc4)nccc3c2)CC1]

### Step 1. Preparation of (1-(cyanomethyl)cyclopropyl)methyl 4-methylbenzenesulfonate

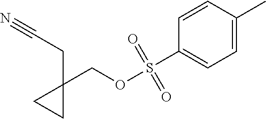 [SMILES: Cc1ccc(S(=O)(=O)OCC2(CC#N)CC2)cc1]

Following the procedure as described for EXAMPLE 207, Step 1, and making variations as required to replace 1-(hydroxymethyl)cyclopropanecarbonitrile with 2-(1-(hydroxymethyl)cyclopropyl)acetonitrile, the title compound was obtained as a colorless solid (1.642 g, 64% yield): ¹H NMR (400 MHz, DMSO-d₆) δ7.81 (d, J=8.3 Hz, 2H), 7.49 (d, J=8.0 Hz, 2H), 3.95 (s, 2H), 2.62 (s, 2H), 2.42 (s, 3H), 0.60 (s, 4H).

### Step 2. Preparation of 2-(1-(((1-chloroisoquinolin-6-yl)oxy)methyl)cyclopropyl)acetonitrile

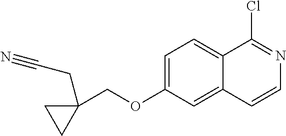 [SMILES: N#CCC1(COc2ccc3c(Cl)nccc3c2)CC1]

Following the procedure as described for EXAMPLE 207, Step 2, and making variations as required to replace (1-cyanocyclopropyl)methyl 4-methylbenzenesulfonate with (1-(cyanomethyl)cyclopropyl)methyl 4-methylbenzenesulfonate, the title compound was obtained as a colorless solid (0.490 g, 58% yield): MS (ES+) m/z 273.6 (M+1), 275.6 (M+1).

### Step 3. Preparation of 2-(1-(((1-((2-chloropyrimidin-5-yl)amino)isoquinolin-6-yl)oxy)methyl)cyclopropyl acetonitrile

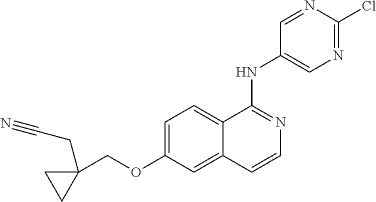 [SMILES: N#CCC1(COc2ccc3c(Nc4cnc(Cl)nc4)nccc3c2)CC1]

Following the procedure as described for EXAMPLE 207, Step 3, and making variations as required to replace 1-(((1-chloroisoquinolin-6-yl)oxy)methyl)cyclopropane-1-carbonitrile with 2-(1-(((1-chloroisoquinolin-6-yl)oxy)methyl)cyclopropyl)acetonitrile, the title compound was obtained as a colorless solid (0.082 g, 13% yield): ¹H NMR (400 MHz, DMSO-d₆) δ9.59 (s, 1H), 9.30 (s, 2H), 8.44 (d, J=9.1 Hz, 1H), 8.00 (d, J=5.8 Hz, 1H), 7.36-7.31 (m, 2H), 7.23 (d, J=5.8 Hz, 1H), 4.06 (s, 2H), 2.82 (s, 2H), 0.77-0.70 (m, 4H); MS (ES+) m/z 366.0 (M+1), 368.0 (M+1).

### Examples 148 and 149

### Synthesis of (1S,3R)-3-((1-((6-chloropyridin-3-yl)amino)isoquinolin-6-yl)oxy)-1-methylcyclohexan-1-ol and (1R,3S)-3-((1-((6-chloropyridin-3-yl)amino)isoquinolin-6-yl)oxy)-1-methylcyclohexan-1-ol

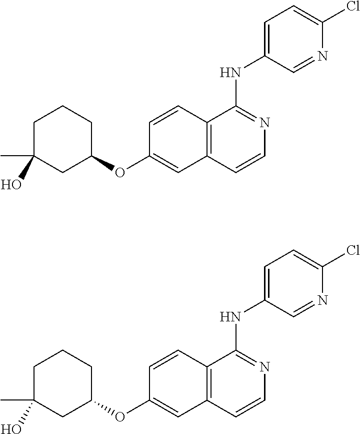 [SMILES: C[C@]1(O)CCC[C@@H](Oc2ccc3c(Nc4ccc(Cl)nc4)nccc3c2)C1 ; C[C@@]1(O)CCC[C@H](Oc2ccc3c(Nc4ccc(Cl)nc4)nccc3c2)C1]

### Step 1. Preparation of 1-methylcyclohexane-1,3-diol

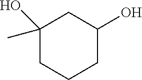

To a solution of 3-hydroxycyclohexan-1-one (0.410 g, 3.59 mmol) in tetrahydrofuran (12 mL) was added a 3.0 M solution of methylmagnesium bromide in tetrahydrofuran (1.40 mL, 4.31 mmol) at 0° C. and the mixture stirred for 2 h. The mixture was diluted with dichloromethane (20 mL) and washed with saturated ammonium chloride (20 mL) and brine (20 mL). The organic phase was dried over anhydrous sodium sulfate and filtered. The filtrate was concentrated in vacuo to give the title compound as a colorless oil (0.430 g, 91% yield): ¹H NMR (400 MHz, CDCl₃) δ 3.84-3.79 (m, 1H), 1.82-1.66 (m, 4H), 1.53-1.38 (m, 4H), 1.21 (s, 3H), OH not observed.

### Step 2. Preparation of (1S,3R)-3-((1-((6-chloropyridin-3-yl)amino)isoquinolin-6-yl)oxy)-1-methylcyclohexan-1-ol and (1R,3S)-3-((1-((6-chloropyridin-3-yl)amino)isoquinolin-6-yl)oxy)-1-methylcyclohexan-1-ol

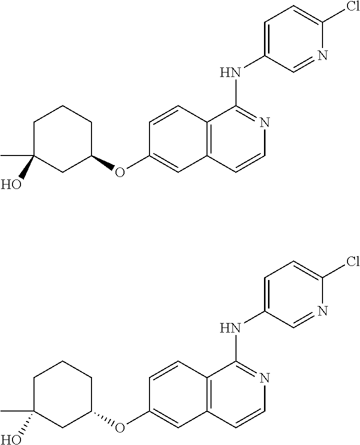 [SMILES: C[C@]1(O)CCC[C@@H](Oc2ccc3c(Nc4ccc(Cl)nc4)nccc3c2)C1 ; C[C@@]1(O)CCC[C@H](Oc2ccc3c(Nc4ccc(Cl)nc4)nccc3c2)C1]

To a mixture of 1-methylcyclohexane-1,3-diol (0.103 g, 0.792 mmol) in N,N-dimethylformamide (5 mL) was added sodium hydride (60% dispersion in mineral oil, 0.048 g, 1.19 mmol) at ambient temperature and the reaction mixture was stirred for 10 minutes. To the mixture was then added N-(6-chloropyridin-3-yl)-6-fluoro-N-((2-(trimethylsilyl)ethoxy)methyl)isoquinolin-1-amine (0.160 g, 0.396 mmol). The reaction mixture was stirred at 60° C. for 16 h. After cooling the ambient temperature, the reaction mixture was quenched by addition of water (20 mL). The mixture was diluted with ethyl acetate (20 mL) and washed with saturated aqueous ammonium chloride (20 mL) and brine (20 mL). The organic phase was dried over anhydrous sodium sulfate, filtered, and the filtrate was concentrated in vacuo. To the obtained residue was added dichloromethane (2 mL) and trifluoroacetic acid (1 mL). The mixture was stirred at ambient temperature for 2 h, and then concentrated in vacuo. Purification and resolution by chiral SFC (Lux Cell-4, 10×250 mm, 5 μm column), eluting with 45% of methanol (containing 10 mM ammonium formate) in supercritical carbon dioxide, afforded the title compounds as single enantiomers as colorless solids. The relative configuration of the title compounds was determined by NOE NMR experiments. First eluting enantiomer (0.029 g, 19% yield): ¹H NMR (400 MHz, DMSO-d₆) δ 9.38 (s, 1H), 8.88 (d, J=2.7 Hz, 1H), 8.42 (dd, J=8.8, 2.1 Hz, 2H), 7.94 (d, J=5.8 Hz, 1H), 7.44 (d, J=8.7 Hz, 1H), 7.29 (d, J=2.4 Hz, 1H), 7.24 (dd, J=9.1, 2.5 Hz, 1H), 7.17 (d, J=5.8 Hz, 1H), 4.60-4.53 (m, 1H), 2.12-2.01 (m, 2H), 1.73-1.69 (m, 1H), 1.57-1.30 (m, 5H), 1.23 (s, 3H), OH not observed; MS (ES+) m/z 384.2 (M+1), 386.2 (M+1). Second eluting enantiomer (0.033 g, 20% yield): ¹H NMR (400 MHz, DMSO-d₆) δ 9.37 (s, 1H), 8.88 (d, J=2.7 Hz, 1H), 8.42 (dd, J=8.8, 2.7 Hz, 2H), 7.94 (d, J=5.8 Hz, 1H), 7.44 (d, J=8.7 Hz, 1H), 7.29 (d, J=2.5 Hz, 1H), 7.24 (dd, J=9.2, 2.5 Hz, 1H), 7.17 (d, J=5.8 Hz, 1H), 4.59-4.53 (m, 1H), 2.13-2.02 (m, 2H), 1.74-1.69 (m, 1H), 1.57-1.27 (m, 5H), 1.23 (d, J=5.7 Hz, 3H), OH not observed; MS (ES+) m/z 384.2 (M+1), 386.2 (M+1).

### Example 150

### Synthesis of N-(6-chloropyridin-3-yl)-6-((1-methyl-1H-pyrazol-3-yl)methoxy)isoquinolin-1-amine

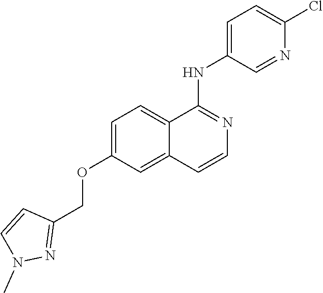 [SMILES: Cn1ccc(COc2ccc3c(Nc4ccc(Cl)nc4)nccc3c2)n1]

Following the procedure as described for EXAMPLE 113 making variations as required to replace 6-iodo-2-oxaspiro[3.3]heptane with 3-(chloromethyl)-1-methyl— pyrazole, the title compound was obtained as a colorless solid (0.036 g, 25% yield): ¹H NMR (400 MHz, DMSO-d₆) δ 9.37 (s, 1H), 8.87 (d, J=2.4 Hz, 1H), 8.49-8.33 (m, 2H), 7.96 (d, J=5.6 Hz, 1H), 7.69 (d, J=2.0 Hz, 1H), 7.51-7.36 (m, 2H), 7.35-7.24 (m, 1H), 7.18 (d, J=5.6 Hz, 1H), 6.38 (d, J=2.0 Hz, 1H), 5.15 (s, 2H), 3.85 (s, 3H); MS (ES+) m/z 366.2 (M+1), 368.2 (M+1).

### Example 151

### Synthesis of N-(6-chloropyridin-3-yl)-6-((1-ethynylcyclopropyl)methoxy)isoquinolin-1-amine

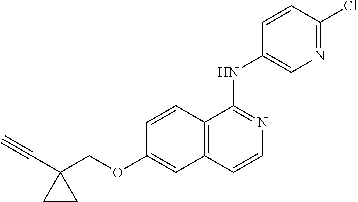 [SMILES: C#CC1(COc2ccc3c(Nc4ccc(Cl)nc4)nccc3c2)CC1]

### Step 1. Preparation of (1-ethynylcyclopropyl)methanol

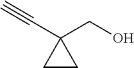

To a solution of 1-ethynylcyclopropane-1-carboxylic acid (0.550 g, 4.99 mmol) in tetrahydrofuran (8 mL) was added a 2.5 M solution of lithium aluminum hydride in tetrahydrofuran (3.0 mL, 7.5 mmol) at 0° C. The reaction mixture was allowed to warm to ambient temperature and stirred for 12 h. The reaction mixture was then cooled to 0° C., and sodium sulfate decahydrate (0.900 g) was added to it. The mixture was filtered through a pad of celite, and the filter cake was washed with tetrahydrofuran (40 mL). The combined filtrate was concentrated in vacuo to afford the title compound as a colorless oil (0.300 g, 63% yield): ¹H NMR (400 MHz, CDCl₃) δ3.50 (s, 2H), 1.96 (s, 1H), 1.91 (s, 1H), 0.99 (d, J=2.4 Hz, 2H), 0.77 (d, J=2.4 Hz, 2H).

### Step 2. Preparation of (1-ethynylcyclopropyl)methyl 4-methylbenzenesulfonate

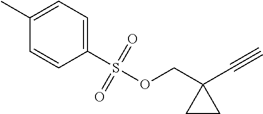 [SMILES: C#CC1(COS(=O)(=O)c2ccc(C)cc2)CC1]

To a solution of (1-ethynylcyclopropyl)methanol (0.300 g, 3.12 mmol) in dichloromethane (6 mL) was added triethylamine (0.316 g, 3.12 mmol), 4-methylbenzenesulfonyl chloride (0.892 g, 4.68 mmol), and 4-dimethylaminopyridine (0.0381 g, 0.312 mmol) at 0° C. The reaction mixture was allowed warm to ambient temperature and stirred for 12 h. To the reaction mixture was then added water (15 mL), and the mixture was extracted with dichloromethane (2×15 mL). The combined organic layers were washed with brine (15 mL), dried over anhydrous sodium sulfate, and filtered. Concentration of the filtrate in vacuo and purification of the obtained residue by silica gel column chromatography, eluting with a gradient of 20 to 40% of ethyl acetate in petroleum ether, afforded the title compound as a colorless oil (0.0800 g, 9% yield): ¹H NMR (400 MHz, CDCl₃) δ7.82 (d, J=8.0 Hz, 2H), 7.35 (d, J=8.0 Hz, 2H), 3.94 (s, 2H), 2.51 (s, 1H), 2.46 (s, 3H), 1.04 (d, J=2.4 Hz, 2H), 0.86-0.80 (m, 2H).

### Step 3. Preparation of N-(6-chloropyridin-3-yl)-6-((1-ethynylcyclopropyl)methoxy)isoquinolin-1-amine

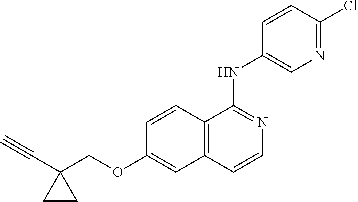 [SMILES: C#CC1(COc2ccc3c(Nc4ccc(Cl)nc4)nccc3c2)CC1]

To a solution of (1-ethynylcyclopropyl)methyl 4-methylbenzenesulfonate (0.0800 g, 0.320 mmol) in N,N-dimethyl formamide (3 mL) was added potassium carbonate (0.0402 g, 0.290 mmol), and 1-((6-chloro-3-pyridyl)amino)isoquinolin-6-ol (0.0789 g, 0.290 mmol). The reaction mixture was heated to 70° C. for 12 h. After cooling to ambient temperature, water (10 mL) was added to the mixture, and the mixture was extracted with ethyl acetate (3×20 mL). The combined organic layers were washed with brine (3×10 mL), dried over anhydrous sodium sulfate, and filtered. The filtrate was concentrated in vacuo. The obtained residue was purified by silica gel column chromatography, eluting with a gradient of 20 to 40% of ethyl acetate in petroleum, and was then purified by reserve-phase preparative HPLC, eluting with a gradient of 16 to 466% of acetonitrile in water containing 0.1% of formic acid, to afford the title compound as a colorless solid (0.0154 g, 12% yield): ¹H NMR (400 MHz, DMSO-d₆) δ9.38 (s, 1H), 8.89 (d, J=2.8 Hz, 1H), 8.54-8.35 (m, 2H), 7.97 (d, J=5.6 Hz, 1H), 7.45 (d, J=8.8 Hz, 1H), 7.33 (dd, J=2.4, 9.2 Hz, 1H), 7.25 (d, J=2.8 Hz, 1H), 7.16 (d, J=5.6 Hz, 1H), 4.06 (s, 2H), 2.83 (s, 1H), 1.01 (d, J=3.6 Hz, 4H); MS (ES+) m/z 350.1 (M+1), 352.1 (M+1).

### Examples 152-155

In a similar manner as described in EXAMPLE 151, Step 3, utilizing the appropriately substituted starting materials and intermediates, the following compounds were prepared:

| | | MS | |
|---|---|---|---|
| | | (ES+) | |
| Example No | Name | m/z | NMR |
| 152 | N-(6-chloropyridin-3- | 384.2 | ¹H NMR (400 MHz, DMSO-d₆) δ 8.80 |
| | yl)-6-((3- | (M + 1), | (s, 1H), 8.59-8.50 (m, 1H), 8.31 (s, |
| | isopropyloxetan-3- | 386.2 | 1H), 7.83 (dd, J = 4.4, 1.6 Hz, 1H), |
| | yl)methoxy)isoquinolin- | (M + 1). | 7.60-7.53 (m, 1H), 7.50 (s, 1H), 7.45- |
| | 1-amine | | 7.39 (m, 1H), 7.25 (d, J = 6.0 Hz, 1H), |
| | | | 4.51 (d, J = 6.0 Hz, 2H), 4.44 (d, J = |
| | | | 6.0 Hz, 2H), 4.30 (s, 2H), 2.25-2.18 |
| | | | (m, 1H), 0.99 (d, J = 6.8 Hz, 6H), NH |
| | | | not observed. |
| 153 | 6-((1-((6-chloropyridin- | 382.2 | ¹H NMR (400 MHz, DMSO-d₆) δ 8.78 |
| | 3-yl)amino)isoquinolin- | (M + 1), | (s, 1H), 8.49 (d, J = 8.4 Hz, 1H), |
| | 6- | 384.2 | 8.31-8.27 (m, 1H), 7.81 (d, J = 2.4 |
| | yl)oxy)spiro[3.3]heptan- | (M + 1). | Hz, 1H), 7.55 (d, J = 8.0 Hz, 1H), |
| | 2-ol | | 7.31-7.20 (m, 3H), 4.80 (q, J = 6.8 |
| | | | Hz, 1H), 4.01 (q, J = 7.2 Hz, 1H), |
| | | | 2.68-2.63 (m, 1H), 2.60-2.53 (m, |
| | | | 1H), 2.44-2.38 (m, 1H), 2.27-2.21 |
| | | | (m, 1H), 2.12-2.06 (m, 2H), 1.94- |
| | | | 1.86 (m, 2H), NH and OH not |
| | | | observed. |
| 154 | 6-((1,4-dioxan-2- | 372.1 | ¹H NMR (400 MHz, DMSO-d₆) δ 9.43 |
| | yl)methoxy)-N-(6- | (M + 1), | (m, 1H), 8.87 (d, J = 2.8 Hz, 1H), |
| | chloropyridin-3- | 374.0 | 8.46-8.39 (m, 2H), 7.95 (d, J = 5.6 |
| | yl)isoquinolin-1-amine | (M + 1). | Hz, 1H), 7.46 (d, J = 8.8 Hz, 1H), |
| | | | 7.30-7.32 (m, 2H), 7.18 (d, J = 6.0 |
| | | | Hz, 1H), 4.12 (d, J = 4.8 Hz, 2H), 3.94 |
| | | | (qd, J = 4.8, 7.6 Hz, 1H), 3.88 (dd, J = |
| | | | 2.0, 11.2 Hz, 1H), 3.81-3.77 (m, 1H), |
| | | | 3.71-3.62 (m, 2H), 3.56-3.50 (m, |
| | | | 1H), 3.48-3.42 (m, 1H). |
| 155 | 3-((1-((6-chloropyridin- | 366.1 | ¹H NMR (400 MHz, DMSO-d₆) δ 9.42 |
| | 3-yl)amino)isoquinolin- | (M + 1), | (s, 1H), 8.89 (d, J = 2.7 Hz, 1H), 8.49 |
| | 6-yl)oxy)-2,2- | 368.1 | (d, J = 9.3 Hz, 1H), 8.43 (dd, J = 8.9, |
| | difluoropropan-1-ol | (M + 1). | 2.7 Hz, 1H), 7.99 (d, J = 5.8 Hz, 1H), |
| | | | 7.46 (d, J = 8.8 Hz, 1H), 7.41 (d, J= |
| | | | 2.3 Hz, 1H), 7.39-7.36 (m, 1H), 7.20 |
| | | | (d, J = 5.8 Hz, 1H), 5.77 (br s), 4.51 |
| | | | (t, J = 12.8 Hz, 2H), 3.82 (t, J = 13.8 |
| | | | Hz, 2H); ¹⁹F NMR (376 MHz, DMSO- |
| | | | d₆) δ −114.0 (s). |

### Example 156 Synthesis of N-(6-chloropyridin-3-yl)-6-((5-methylisoxazol-4-yl)methoxy)isoquinolin-1-amine

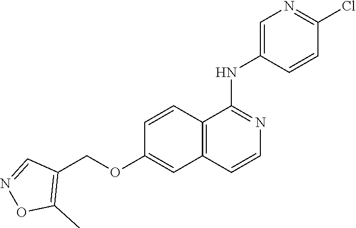 [SMILES: Cc1oncc1COc1ccc2c(Nc3ccc(Cl)nc3)nccc2c1]

### Step 1. Preparation of 4-(((1-chloroisoquinolin-6-yl)oxy)methyl)-5-methylisoxazole

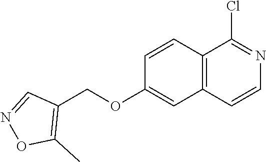 [SMILES: Cc1oncc1COc1ccc2c(Cl)nccc2c1]

A mixture of 1-chloroisoquinolin-6-ol (0.290 g, 1.61 mmol), (5-methylisoxazol-4-yl)methanol (0.219 g, 1.94 mmol), and triphenylphosphine (0.508 g, 1.94 mmol) in tetrahydrofuran (3.2 mL) was stirred for 10 minutes at the ambient temperature. The mixture was then cooled to 0° C., and diethyl azodicarboxylate (0.30 mL, 1.94 mmol) was slowly added to it. The mixture was allowed to warm up to ambient temperature and stirred for 2 h. The mixture was diluted with saturated sodium bicarbonate solution (20 mL) and extracted with ethyl acetate (3×20 mL). The combined organic layers were dried over anhydrous sodium sulfate and filtered. The filtrate was concentrated in vacuo and the obtained residue was purified by silica gel column chromatography, eluting with a gradient of 0 to 60% of ethyl acetate in heptane, to provide the title compound as a colorless solid (0.288 g, 65% yield): ¹H NMR (400 MHz, CDCl₃) δ 8.31 (s, 1H), 8.27 (d, J=9.2 Hz, 1H), 8.22 (d, J=5.7 Hz, 1H), 7.50 (d, J=5.7 Hz, 1H), 7.31 (dd, J=9.3, 2.5 Hz, 1H), 7.16 (d, J=2.5 Hz, 1H), 5.01 (s, 2H), 2.51 (s, 3H); MS (ES+) m/z 275.5 (M+1), 277.5 (M+1).

### Step 2. Preparation of N-(6-chloropyridin-3-yl)-6-((5-methylisoxazol-4-yl)methoxy)isoquinolin-1-amine

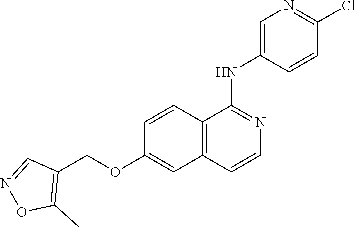 [SMILES: Cc1oncc1COc1ccc2c(Nc3ccc(Cl)nc3)nccc2c1]

To a solution of 4-(((1-chloroisoquinolin-6-yl)oxy)methyl)-5-methylisoxazole (0.130 g, 0.473 mmol) in 1,4-dioxane (6.8 mL) was added 5-amino-2-chloropyridine (0.064 g, 0.497 mmol), tris(dibenzylideneacetone)dipalladium(0) (0.043 g, 0.047 mmol), 2-dicyclohexylphosphino-2′,6′-dimethoxy-1,1′-biphenyl (0.039 g, 0.095 mmol), and potassium phosphate tribasic (0.402 g, 1.89 mmol). The mixture was degassed by passing a stream of nitrogen through it for 5 minutes, and then the reaction mixture was heated to 70° C. for 2 h. After cooling to ambient temperature, the reaction mixture was filtered through celite, and the filtrate was concentrated in vacuo. The residue was purified by silica gel column chromatography, eluting with a gradient of 0 to 100% of ethyl acetate in heptane. The obtained residue was then purified by reverse-phase column chromatography, eluting with a gradient of 5 to 60% of acetonitrile in water (containing 0.5% of formic acid), to provide the title compound as a colorless solid (0.014 g, 7% yield): ¹H NMR (400 MHz, CDCl₃) δ8.50 (d, J=2.8 Hz, 1H), 8.32 (q, J=3.0 Hz, 2H), 8.03 (d, J=5.9 Hz, 1H), 7.89 (d, J=9.1 Hz, 1H), 7.31 (d, J=8.7 Hz, 1H), 7.20 (dd, J=9.1, 2.5 Hz, 1H), 7.14-7.13 (m, 2H), 5.00 (s, 2H), 2.51 (s, 3H), NH not observed; MS (ES+) m/z 367.0 (M+1), 369.0 (M+1).

### Example 157

### Synthesis of N-(6-chloropyridin-3-yl)-6-(isoxazol-5-ylmethoxy)isoquinolin-1-amine

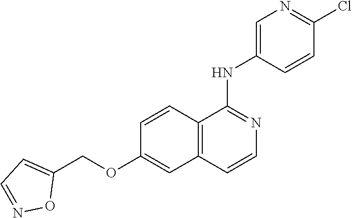 [SMILES: Clc1ccc(Nc2nccc3cc(OCc4ccno4)ccc23)cn1]

### Step 1. Preparation of 5-(((1-chloroisoquinolin-6-yl)oxy)methyl)isoxazole

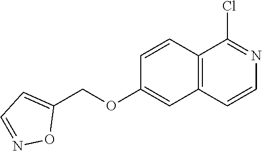 [SMILES: Clc1nccc2cc(OCc3ccno3)ccc12]

Following the procedure as described for EXAMPLE 156, Step 1, and making variations as required to replace (5-methylisoxazol-4-yl)methanol with 5-isoxazolemethanol, the title compound was obtained as a colorless solid (0.376 g, quantitative yield): ¹H NMR (400 MHz, CDCl₃) δ 8.29-8.26 (m, 2H), 8.21 (d, J=5.7 Hz, 1H), 7.50 (d, J=5.7 Hz, 1H), 7.35 (dd, J=9.3, 2.5 Hz, 1H), 7.16 (d, J=2.5 Hz, 1H), 6.42-6.42 (m, 1H), 5.35 (s, 2H); MS (ES+) m/z 261.6 (M+1), 263.6 (M+1).

### Step 2. Preparation of N-(6-chloropyridin-3-yl)-6-(isoxazol-5-ylmethoxy)isoquinolin-1-amine

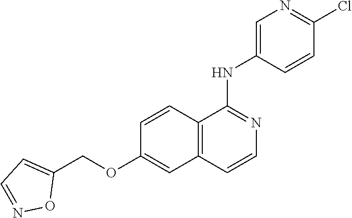 [SMILES: Clc1ccc(Nc2nccc3cc(OCc4ccno4)ccc23)cn1]

Following the procedure as described for EXAMPLE 156, Step 2, and making variations as required to replace 4-(((1-chloroisoquinolin-6-yl)oxy)methyl)-5-methylisoxazole with 5-(((1-chloroisoquinolin-6-yl)oxy)methyl)isoxazole, the title compound was obtained as a colorless solid (0.018 g, 7% yield): ¹H NMR (400 MHz, DMSO-d₆) δ9.42 (s, 1H), 8.88 (d, J=2.8 Hz, 1H), 8.64 (d, J=1.8 Hz, 1H), 8.48 (d, J=9.3 Hz, 1H), 8.42 (dd, J=8.8, 2.9 Hz, 1H), 7.99 (d, J=5.7 Hz, 1H), 7.47-7.45 (m, 2H), 7.37 (dd, J=9.2, 2.4 Hz, 1H), 7.20 (d, J=5.8 Hz, 1H), 6.73 (d, J=1.8 Hz, 1H), 5.49 (s, 2H); MS (ES+) m/z 353.4 (M+1), 355.4 (M+1).

### Example 158

### Synthesis of N-(6-chloropyridin-3-yl)-6-((2-(pyridin-3-ylmethyl)oxazol-5-yl)methoxy)isoquinolin-1-amine

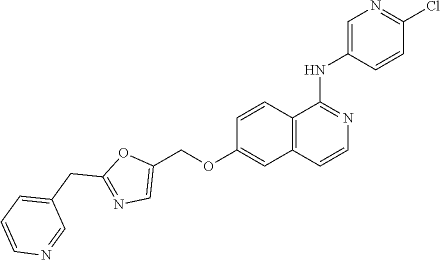 [SMILES: Clc1ccc(Nc2nccc3cc(OCc4cnc(Cc5cccnc5)o4)ccc23)cn1]

### Step 1. Preparation of N-(prop-2-yn-1-yl)-2-(pyridin-3-yl)acetamide

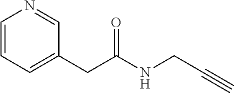

To a solution of 2-propyn-1-amine (0.26 mL, 4.01 mmol) and 3-pyridylacetic acid (0.500 g, 3.65 mmol) in dichloromethane (5.6 mL) was added benzotriazol-1-yloxytripyrrolidinophosphonium hexafluorophosphate (2.09 mg, 4.01 mmol) and N,N-diisopropylethylamine (1.9 mL, 10.9 mmol), and the reaction mixture was stirred at ambient temperature for 16 h. The reaction mixture was then diluted with methanol (5 mL) and stirred for 30 minutes. After addition of saturated sodium bicarbonate solution (30 mL), the mixture was extracted with dichloromethane (3×30 mL). The combined organic phase was washed with brine (30 mL), dried over anhydrous sodium sulfate, and filtered. The filtrate was concentrated in vacuo to provide the title compound as a colorless solid (2.25 g, quantitative yield) which was used for the next step without further purification: MS (ES+) m/z 175.2 (M+1).

### Step 2. Preparation of (2-(pyridin-3-ylmethyl)oxazol-5-yl)methyl acetate

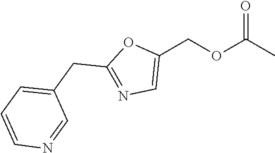 [SMILES: CC(=O)OCc1cnc(Cc2cccnc2)o1]

To a solution of N-(prop-2-yn-1-yl)-2-(pyridin-3-yl)acetamide (2.25 g, 12.9 mmol) in acetic acid (15 mL) was added iodobenzene diacetate (2.080 g, 6.46 mmol), and the reaction mixture was stirred at 90° C. for 20 h. After cooling to ambient temperature, the reaction mixture was diluted with ethyl acetate (20 mL), and saturated sodium bicarbonate solution was added to it until pH 8 was reached. The mixture was extracted with ethyl acetate (3×100 mL), and the combined organic layers were dried over anhydrous sodium sulfate and filtered. The filtrate was concentrated in vacuo to afford a residue which was purified by silica gel column chromatography, eluting with a gradient of 0 to 100% of ethyl acetate in heptane and 0 to 20% of methanol in dichloromethane, to provide the title compound as a dark oil (0.849 g, 20% yield): ¹H NMR (400 MHz, CDCl₃) δ 8.59 (d, J=1.7 Hz, 1H), 8.55 (dd, J=4.8, 1.3 Hz, 1H), 7.69-7.66 (m, 1H), 7.31-7.29 (m, 1H), 7.04 (s, 1H), 5.06 (s, 2H), 4.14 (s, 2H), 2.09 (s, 3H); MS (ES+) m/z 233.2 (M+1).

### Step 3. Preparation of (2-(pyridin-3-ylmethyl)oxazol-5-yl)methanol

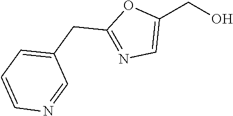 [SMILES: OCc1cnc(Cc2cccnc2)o1]

To a solution of (2-(pyridin-3-ylmethyl)oxazol-5-yl)methyl acetate (0.489 g, 2.11 mmol) in methanol (4 mL) was added potassium carbonate (0.582 g, 4.21 mmol) and the reaction mixture was stirred at ambient temperature for 4 h. The mixture was concentrated in vacuo to afford a residue which was purified by silica gel column chromatography, eluting with a gradient of 0 to 100% of ethyl acetate in heptane and 0 to 15% of methanol in dichloromethane, to provide the title compound as a yellowish oil (0.101 g, 24% yield): ¹H NMR (400 MHz, CDCl₃) δ8.53-8.50 (m, 2H), 7.67 (d, J=7.9 Hz, 1H), 7.30-7.27 (m, 1H), 6.91 (s, 1H), 4.63 (s, 2H), 4.11 (s, 2H), OH not observed; MS (ES+) m/z 191.2 (M+1).

### Step 4. Preparation of 5-(((1-chloroisoquinolin-6-yl)oxy)methyl)-2-(pyridin-3-ylmethyl)oxazole

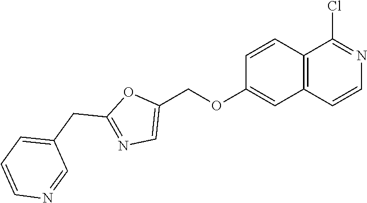 [SMILES: Clc1nccc2cc(OCc3cnc(Cc4cccnc4)o3)ccc12]

Following the procedure as described for EXAMPLE 156, Step 1, and making variations as required to replace (5-methylisoxazol-4-yl)methanol with (2-(pyridin-3-ylmethyl)oxazol-5-yl)methanol, the title compound was obtained as a colorless solid (0.020 g, 27% yield): ¹H NMR (400 MHz, CDCl₃) δ 8.61 (d, J=1.6 Hz, 1H), 8.55 (d, J=4.8 Hz, 1H), 8.24 (dd, J=15.2, 7.5 Hz, 2H), 7.69-7.67 (m, 1H), 7.49 (d, J=5.7 Hz, 1H), 7.32 (dd, J=9.3, 2.5 Hz, 1H), 7.30-7.28 (m, 1H), 7.18-7.16 (m, 2H), 5.17 (s, 2H), 4.18 (s, 2H); MS (ES+) m/z 352.2 (M+1), 354.2 (M+1).

### Step 5. Preparation of N-(6-chloropyridin-3-yl)-6-((2-(pyridin-3-ylmethyl)oxazol-5-yl)methoxy)isoquinolin-1-amine

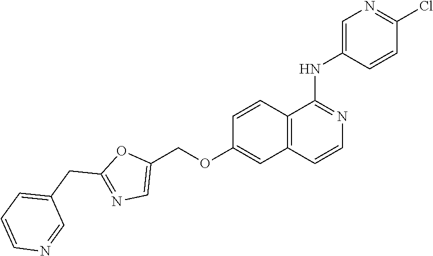 [SMILES: Clc1ccc(Nc2nccc3cc(OCc4cnc(Cc5cccnc5)o4)ccc23)cn1]

Following the procedure as described for EXAMPLE 156, Step 2, and making variations as required to replace 4-(((1-chloroisoquinolin-6-yl)oxy)methyl)-5-methylisoxazole with 5-(((1-chloroisoquinolin-6-yl)oxy)methyl)-2-(pyridin-3-ylmethyl)oxazole, the title compound was obtained as a colorless solid (0.040 g, 25% yield): ¹H NMR (400 MHz, CDCl₃) δ8.60 (d, J=2.0 Hz, 1H), 8.56 (dd, J=4.8, 1.6 Hz, 1H), 8.54 (d, J=2.9 Hz, 1H), 8.39 (dd, J=8.7, 2.9 Hz, 1H), 8.06 (d, J=5.8 Hz, 1H), 7.91 (d, J=9.1 Hz, 1H), 7.70-7.67 (m, 1H), 7.34 (d, J=8.7 Hz, 1H), 7.31-7.30 (m, 1H), 7.24 (dd, J=9.1, 2.6 Hz, 1H), 7.16 (s, 2H), 7.13 (d, J=5.9 Hz, 1H), 5.17 (s, 2H), 4.18 (s, 2H), NH not observed; MS (ES+) m/z 444.0 (M+1), 446.0 (M+1).

### Examples 159 and 160

### Synthesis of (R)—N-(6-chloropyridin-3-yl)-6-(2-(1-methyl-1H-pyrazol-4-yl)propoxy)isoquinolin-1-amine and (S)—N-(6-chloropyridin-3-yl)-6-(2-(1-methyl-1H-pyrazol-4-yl)propoxy)isoquinolin-1-amine

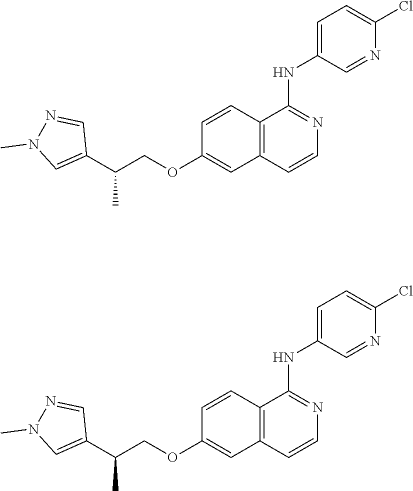 [SMILES: C[C@@H](COc1ccc2c(Nc3ccc(Cl)nc3)nccc2c1)c1cnn(C)c1 ; C[C@H](COc1ccc2c(Nc3ccc(Cl)nc3)nccc2c1)c1cnn(C)c1]

### Step 1. Preparation of 2-(1-methyl-1H-pyrazol-4-yl)propan-1-ol

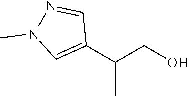

To a solution of methyl 2-(1-methyl-1H-pyrazol-4-yl)propanoate (0.500 g, 2.97 mmol) in tetrahydrofuran (20 mL) was added a 1.0 M solution of lithium aluminum hydride in tetrahydrofuran (2.97 mL, 2.97 mmol) at 0° C. The mixture was allowed to warm to ambient temperature and stirred for 2 h. The reaction mixture was quenched by addition of sodium sulfate decahydrate and filtered. The filtrate was concentrated in vacuo to give the title compound as a colorless gum (0.400 g, 88% yield): ¹H NMR (400 MHz, DMSO-d₆) δ7.44 (s, 1H), 7.25 (s, 1H), 4.62 (t, J=5.2 Hz, 1H), 3.75 (s, 3H), 3.47-3.42 (m, 1H), 3.31-3.25 (m, 1H), 2.68 (m, 1H), 1.13 (d, J=7.2 Hz, 3H).

### Step 2. Preparation of 1-chloro-6-(2-(1-methyl-1H-pyrazol-4-yl)propoxy)isoquinoline

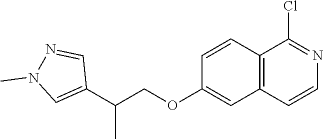 [SMILES: CC(COc1ccc2c(Cl)nccc2c1)c1cnn(C)c1]

Following the procedure as described for EXAMPLE 156, Step 1 and making variations as required to replace (5-methylisoxazol-4-yl)methanol with 2-(1-methyl-1H-pyrazol-4-yl)propan-1-ol, the title compound were obtained as a yellowish solid (0.400 g, 63% yield): ¹H NMR (400 MHz, DMSO-d₆) δ 8.21-8.13 (m, 2H), 7.75 (d, J=5.6 Hz, 1H), 7.62 (s, 1H), 7.51 (d, J=2.4 Hz, 1H), 7.46-7.39 (m, 2H), 4.21-4.14 (m, 1H), 4.12-4.05 (m, 1H), 3.79 (s, 3H), 3.24-3.17 (m, 1H), 1.32 (d, J=6.8 Hz, 3H).

### Step 3. Preparation of (R)—N-(6-chloropyridin-3-yl)-6-(2-(1-methyl-1H-pyrazol-4-yl)propoxy)isoquinolin-1-amine and (S)—N-(6-chloropyridin-3-yl)-6-(2-(1-methyl-1H-pyrazol-4-yl)propoxy)isoquinolin-1-amine

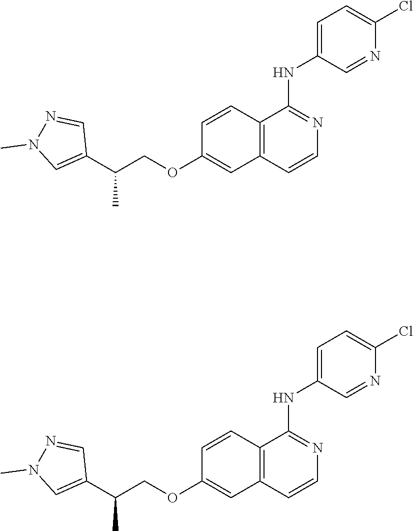 [SMILES: C[C@@H](COc1ccc2c(Nc3ccc(Cl)nc3)nccc2c1)c1cnn(C)c1 ; C[C@H](COc1ccc2c(Nc3ccc(Cl)nc3)nccc2c1)c1cnn(C)c1]

To a solution of 1-chloro-6-(2-(1-methyl-1H-pyrazol-4-yl)propoxy)isoquinoline (0.160 g, 0.530 mmol) and 6-chloropyridin-3-amine (0.0950 g, 0.742 mmol) in propan-2-ol (10 mL) was added a 4 M solution of hydrogen chloride in dioxane (1.00 mL, 4.00 mmol) and the reaction mixture was stirred at 70° C. for 16 h. After cooling to ambient temperature, the mixture was diluted with water (20 mL) and extracted with ethyl acetate (3×20 mL). The combined organic phase was washed with brine (3×10 mL), dried over anhydrous sodium sulfate, and filtered. The filtrate was concentrated in vacuo and the obtained residue was purified by reverse-phase preparative HPLC (Phenomenex Luna C18 150 mm×25 mm, 10 μm column), eluting with a gradient of 16 to 46% of acetonitrile in water (containing 0.5% of formic acid) to provide the title compounds as a mixture of enantiomers (0.140 g, 65% yield). Resolution of the enantiomers by chiral SFC (Daicel Chiralpak AS 250×30 mm, 10 μm column), eluting with 60% of acetonitrile and isopropanol (containing 0.1% ammonium hydroxide) in supercritical carbon dioxide, afforded the title compounds as single enantiomers as colorless solids. First eluting enantiomer (0.050 g, 36% yield): ¹H NMR (400 MHz, DMSO-d₆) δ 9.37 (s, 1H), 8.88 (d, J=2.8 Hz, 1H), 8.46-8.41 (m, 2H), 7.96 (d, J=6.0 Hz, 1H), 7.63 (s, 1H), 7.45 (d, J=8.8 Hz, 1H), 7.42 (s, 1H), 7.32-7.28 (m, 2H), 7.18 (d, J=5.6 Hz, 1H), 4.19-4.14 (m, 1H), 4.09-4.04 (m, 1H), 3.80 (s, 3H), 3.23-3.17 (m, 1H), 1.33 (d, J=6.8 Hz, 3H); MS (ES+) m/z 394.1 (M+1), 396.1 (M+1). Second eluting enantiomer (0.058 g, 42% yield): ¹H NMR (400 MHz, DMSO-d₆) δ9.41 (s, 1H), 8.87 (d, J=2.0 Hz 1H), 8.45-0.839 (m, 2H), 7.93 (d, J=4.4 Hz, 1H), 7.62 (s, 1H), 7.45 (d, J=8.8 Hz, 1H), 7.41 (s, 1H), 7.33-7.27 (m, 2H), 7.18 (d, J=5.6 Hz, 1H), 4.18-4.13 (m, 1H), 4.09-4.03 (m, 1H), 3.79 (s, 3H), 3.23-3.17 (m, 1H), 1.33 (d, J=6.8 Hz, 3H); MS (ES+) m/z 394.1 (M+1), 396.1 (M+1).

### Example 161

### Synthesis of N-(6-chloropyridin-3-yl)-6-((2,2-dimethylpent-3-yn-1-yl)oxy)isoquinolin-1-amine

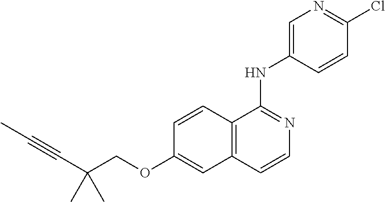 [SMILES: CC#CC(C)(C)COc1ccc2c(Nc3ccc(Cl)nc3)nccc2c1]

### Step 1. Preparation of 2,2-dimethylpent-3-ynoic acid

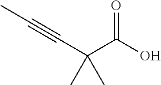

To a solution of 2,2-dimethylpent-4-ynoic acid (0.800 g, 6.34 mmol) in dimethyl sulfoxide (12.0 mL) was added potassium tert-butoxide (1.42 g, 12.7 mmol) at ambient temperature, and the mixture was heated to 75° C. for 16 h. The reaction mixture was diluted with water (10 mL) and then acidified with 1 M hydrochloric acid to pH=3. The mixture was extracted with ethyl acetate (3×20 mL). The combined organic layers were washed with brine (3×30 mL), dried over anhydrous sodium sulfate, and filtered. The filtrate was concentrated in vacuo to afford the title compound as a colorless solid (0.780 g, 98% yield): ¹H NMR (400 MHz, CDCl₃) δ 1.84 (s, 3H), 1.49 (s, 6H), OH not observed.

### Step 2. Preparation of 2,2-dimethylpent-3-yn-1-ol

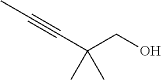

Following the procedure as described for EXAMPLE 151, Step 1, and making variations as required to replace 1-ethynylcyclopropane-1-carboxylic acid with 2,2-dimethylpent-3-ynoic acid, the title compound was obtained as a colorless oil (0.230 g, 65% yield): ¹H NMR (400 MHz, CDCl₃) 93.36 (s, 2H), 1.81 (s, 3H), 1.18 (s, 6H), OH not observed.

### Step 3. Preparation of N-(6-chloropyridin-3-yl)-6-((2,2-dimethylpent-3-yn-1-yl)oxy)isoquinolin-1-amine

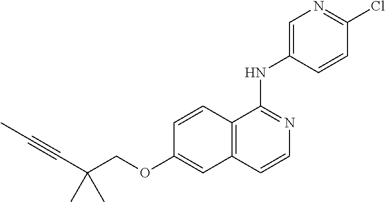 [SMILES: CC#CC(C)(C)COc1ccc2c(Nc3ccc(Cl)nc3)nccc2c1]

Following the procedure as described for EXAMPLE 76, Step 3, and making variations as required to replace phenylmethanol with 2,2-dimethylpent-3-yn-1-ol, the title compound was obtained as a colorless solid (0.021 g, 61% yield): ¹H NMR (400 MHz, DMSO-d₆) δ9.38 (s, 1H), 8.89 (d, J=2.8 Hz, 1H), 8.46-8.41 (m, 2H), 7.96 (d, J=5.6 Hz, 1H), 7.44 (d, J=8.8 Hz, 1H), 7.33-7.31 (m, 2H), 7.19 (d, J=5.6 Hz, 1H), 3.97 (s, 2H), 1.76 (s, 3H), 1.30 (s, 6H); MS (ES+) m/z 366.1 (M+1), 368.1 (M+1).

### Example 162

### Synthesis of 2-chloro-N³-methyl-N⁵-(6-((3-methyloxetan-3-yl)methoxy)isoquinolin-1-yl)pyridine-3,5-diamine

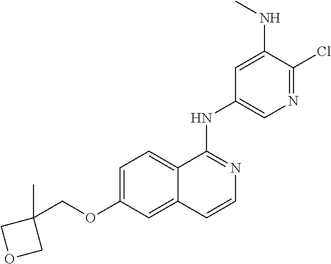 [SMILES: CNc1cc(Nc2nccc3cc(OCC4(C)COC4)ccc23)cnc1Cl]

### Step 1. Preparation of tert-butyl (2-chloro-5-nitropyridin-3-yl)carbamate

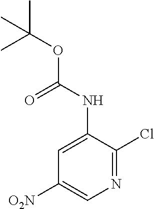 [SMILES: CC(C)(C)OC(=O)Nc1cc([N+](=O)[O-])cnc1Cl]

To a solution of 2-chloro-5-nitronicotinic acid (4.00 g, 19.8 mmol) in toluene (80 mL) was added diphenylphosphoryl azide (8.15 g, 29.6 mmol), triethylamine (2.80 g, 27.7 mmol), and 2-methylpropan-2-ol (14.6 g, 197 mmol). The mixture was stirred at 110° C. for 12 h. After cooling to ambient temperature, saturated sodium bicarbonate (100 mL) was added to the mixture. The mixture was extracted with ethyl acetate (3×100 mL). The combined organic phase was dried over anhydrous sodium sulfate, filtered, and the filtrate was concentrated in vacuo. The residue was purified by silica gel column chromatography, eluting with 20% of ethyl acetate in petroleum ether, to provide the title compound as a yellowish solid (2.50 g, 23% yield): ¹H NMR (400 MHz, CDCl₃) δ9.40 (s, 1H), 8.94 (d, J=2.4 Hz, 1H), 8.90 (d, J=2.4 Hz, 1H), 1.51 (s, 9H).

### Step 2. Preparation of tert-butyl (2-chloro-5-nitropyridin-3-yl)(methyl)carbamate

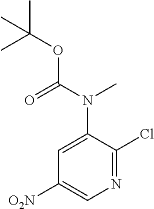 [SMILES: CN(C(=O)OC(C)(C)C)c1cc([N+](=O)[O-])cnc1Cl]

To a solution of tert-butyl (2-chloro-5-nitropyridin-3-yl)carbamate (1.00 g, 3.65 mmol) in tetrahydrofuran (20 mL) was added sodium hydride (60% dispersion in mineral oil, 0.440 g, 11.0 mmol) and iodomethane (1.55 g, 10.9 mmol) at 0° C. The reaction mixture was allowed to warm to ambient temperature and stirred for 16 h. The reaction mixture was quenched with water (20 mL) and extracted with ethyl acetate (3×20 mL). The combined organic phase was washed with brine (20 mL), dried over sodium sulfate, and filtered. The filtrate was concentrated in vacuo and the obtained residue was purified by silica gel column chromatography, eluting with a gradient of 0 to 10% of ethyl acetate in petroleum ether, to provide the title compound as a colorless solid (0.650 g, 62% yield): ¹H NMR (400 MHz, CDCl₃) δ9.18 (d, J=2.4 Hz, 1H), 8.83 (d, J=2.4 Hz, 1H), 3.15 (s, 3H), 1.59-1.17 (m, 9H).

### Step 3. Preparation of tert-butyl (5-amino-2-chloropyridin-3-yl)(methyl)carbamate

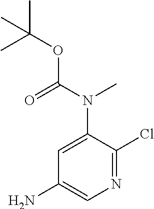 [SMILES: CN(C(=O)OC(C)(C)C)c1cc(N)cnc1Cl]

To a mixture of tert-butyl (2-chloro-5-nitropyridin-3-yl)(methyl)carbamate (0.622 g, 2.16 mmol), iron powder (0.362 g, 6.49 mmol), and ammonium chloride (0.578 g, 10.8 mmol) was added methanol (40 mL) and water (20 mL). The reaction mixture was stirred at 70° C. for 1 h and then filtered through a pad of celite. The filtrate was concentrated in vacuo. The obtained residue was diluted with water (20 mL) and extracted with ethyl acetate (3×30 mL). The combined organic phase was washed with saturated sodium bicarbonate (20 mL) and brine (20 mL), dried over anhydrous sodium sulfate, and filtered. The filtrate was concentrated in vacuo and the obtained residue was purified by silica gel column chromatography, eluting with a gradient of 0 to 100% of ethyl acetate in petroleum ether, to provide the title compound as a yellow oil (0.230 g, 39% yield): ¹H NMR (400 MHz, CDCl₃) δ7.64 (s, 1H), 6.95 (d, J=2.8 Hz, 1H), 5.59 (s, 2H), 1.45 (s, 3H), 1.30 (s, 9H).

### Step 4. Preparation of tert-butyl (2-chloro-5-((6-((3-methyloxetan-3-yl)methoxy)isoquinolin-1-yl)amino)pyridin-3-yl)(methyl)carbamate

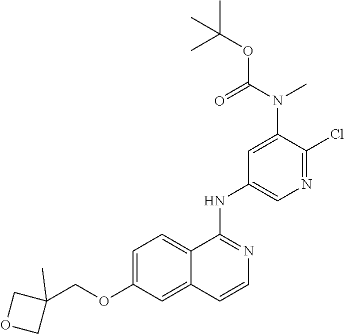 [SMILES: CN(C(=O)OC(C)(C)C)c1cc(Nc2nccc3cc(OCC4(C)COC4)ccc23)cnc1Cl]

A mixture of tert-butyl (5-amino-2-chloropyridin-3-yl)(methyl)carbamate (0.100 g, 0.388 mmol), 1-chloro-6-((3-methyloxetan-3-yl)methoxy)isoquinoline (0.102 g, 0.388 mmol), (2-dicyclohexylphosphino-2′,6′-diisopropoxy-1,1′-biphenyl)[2-(2′-amino-1,1′-biphenyl)]palladium(II) methanesulfonate (0.032 g, 0.039 mmol), and cesium carbonate (0.379 g, 1.16 mmol) in 2-methylbutan-2-ol (1 mL) was degassed by passing a stream of nitrogen through it for 5 minutes. The reaction mixture was heated to 100° C. for 2 h in a microwave reactor. After cooling to ambient temperature, the reaction mixture was diluted with water (20 mL) and extracted with ethyl acetate (3×30 mL). The combined organic phase was washed with brine (20 mL), dried over anhydrous sodium sulfate, and filtered. The filtrate was concentrated in vacuo and the obtained residue was purified by silica gel column chromatography, eluting with a gradient of 0 to 50% of ethyl acetate in petroleum ether, to provide the title compound as a colorless solid (0.160 g, 85% yield): ¹H NMR (400 MHz, DMSO-d₆) δ 9.47 (s, 1H), 8.87 (s, 1H), 8.55-8.41 (m, 2H), 8.01 (d, J=5.6 Hz, 1H), 7.42-7.32 (m, 2H), 7.22 (d, J=6.0 Hz, 1H), 4.61-4.47 (m, 2H), 4.36 (d, J=6.0 Hz, 2H), 4.28-4.21 (m, 2H), 3.12 (s, 3H), 1.43 (s, 3H), 1.36-1.31 (br s, 9H).

### Step 5. Preparation of 2-chloro-N³-methyl-N⁵-(6-((3-methyloxetan-3-yl)methoxy)isoquinolin-1-yl)pyridine-3,5-diamine

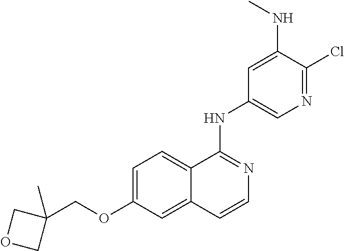 [SMILES: CNc1cc(Nc2nccc3cc(OCC4(C)COC4)ccc23)cnc1Cl]

To a solution of tert-butyl (2-chloro-5-((6-((3-methyloxetan-3-yl)methoxy)isoquinolin-1-yl)amino)pyridin-3-yl)(methyl)carbamate (0.130 g, 0.268 mmol) in dichloromethane (5 mL) was added trifluoroacetic acid (0.8 mL, 10.8 mmol). The reaction mixture was diluted with water (20 mL) and extracted with ethyl acetate (3×30 mL). The combined organic phase was washed with saturated sodium bicarbonate solution (20 mL) and brine (20 mL), and dried over anhydrous sodium sulfate. Filtration and concentration of the filtrate in vacuo gave a residue which was purified by reverse-phase preparative HPLC (Phenomenex Luna C18 150 mm×25 mm, 10 μm column), eluting with a gradient of 14% to 45% of acetonitrile in water (containing 0.5% of formic acid), to provide the title compound as a colorless solid (0.056 g, 53% yield): ¹H NMR (400 MHz, DMSO-d₆) δ 8.50 (d, J=9.2 Hz, 1H), 8.33-8.07 (m, 1H), 7.90 (d, J=2.0 Hz, 1H), 7.56 (s, 1H), 7.45-7.26 (m, 2H), 7.18 (d, J=6.0 Hz, 1H), 5.74 (s, 1H), 4.55 (d, J=5.6 Hz, 2H), 4.36 (d, J=6.0 Hz, 2H), 4.24 (s, 2H), 2.78 (d, J=4.4 Hz, 3H), 1.42 (s, 3H), one NH not observed; MS (ES+) m/z 385.1 (M+1), 387.1 (M+1).

### Example 163

### Synthesis of 2-chloro-N⁵-(6-((3-methyloxetan-3-yl)methoxy)isoquinolin-1-yl)pyridine-3,5-diamine

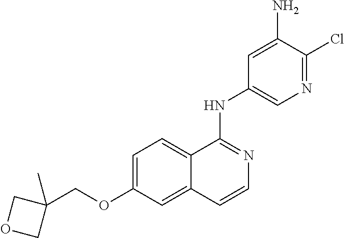 [SMILES: CC1(COc2ccc3c(Nc4cnc(Cl)c(N)c4)nccc3c2)COC1]

### Step 1. Preparation of methyl 5-amino-2-chloronicotinate

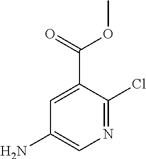

Following the procedure as described for EXAMPLE 162, Step 3, and making variations as required to replace tert-butyl (2-chloro-5-nitropyridin-3-yl)(methyl)carbamate with methyl 2-chloro-5-nitronicotinate, the title compound was obtained as a yellow solid (1.30 g, 75% yield): ¹H-NMR (400 MHz, DMSO-d₆) δ 7.85 (d, J=2.8 Hz, 1H), 7.36 (d, J=2.8 Hz, 1H), 5.80 (s, 2H), 3.83 (s, 3H).

### Step 2. Preparation of methyl 2-chloro-5-((6-hydroxyisoquinolin-1-yl)amino)nicotinate

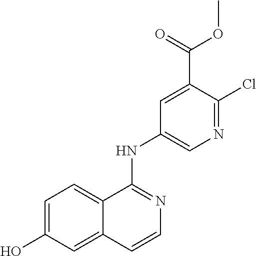 [SMILES: COC(=O)c1cc(Nc2nccc3cc(O)ccc23)cnc1Cl]

To a solution of methyl 5-amino-2-chloronicotinate (0.250 g, 1.34 mmol) and 1-chloroisoquinolin-6-ol (0.290 g, 1.61 mmol) in propan-2-ol (15 mL) was added a 4 M solution of hydrogen chloride in dioxane (1.50 mL, 6.00 mmol). The reaction mixture was stirred at 70° C. for 12 h. After cooling to ambient temperature, the mixture was diluted with water (10 mL) and extracted with ethyl acetate (3×15 mL). The combined organic phase was washed with brine (3×10 mL), dried over anhydrous sodium sulfate, and filtered. The filtrate was concentrated in vacuo and the obtained residue was purified by silica gel column chromatography, eluting with 25% of methanol in ethyl acetate, to provide the title compound as a yellow solid (0.360 g, 81% yield): ¹H NMR (400 MHz, DMSO-d₆) δ8.91 (s, 1H), 8.63-8.54 (m, 2H), 7.70-7.61 (m, 1H), 7.36-7.16 (m, 4H), 3.91 (s, 3H), OH not observed

### Step 3. Preparation of methyl 2-chloro-5-((6-((3-methyloxetan-3-yl)methoxy)isoquinolin-1-yl)amino)nicotinate

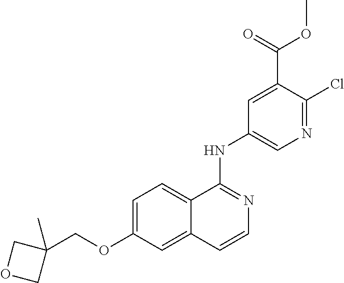 [SMILES: COC(=O)c1cc(Nc2nccc3cc(OCC4(C)COC4)ccc23)cnc1Cl]

Following the procedure as described for EXAMPLE 36, Step 2, and making variations as required to replace 1-chloroisoquinolin-6-ol with methyl 2-chloro-5-((6-hydroxyisoquinolin-1-yl)amino)nicotinate, the title compound was obtained as a colorless solid (0.130 g, 43% yield): ¹H-NMR (400 MHz, DMSO-d₆) δ 9.63-9.51 (m, 1H), 9.20-9.10 (m, 1H), 8.87 (d, J=2.4 Hz, 1H), 8.47 (d, J=8.8 Hz, 1H), 8.01 (d, J=6.0 Hz, 1H), 7.40-7.35 (m, 2H), 7.24 (d, J=5.6 Hz, 1H), 4.55 (d, J=5.6 Hz, 2H), 4.35 (d, J=6.0 Hz, 2H), 4.23 (s, 2H), 3.91 (s, 3H), 1.42 (s, 3H).

### Step 4. Preparation of 2-chloro-5-((6-((3-methyloxetan-3-yl)methoxy)isoquinolin-1-yl)amino)nicotinic acid

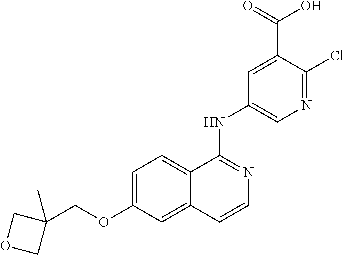 [SMILES: CC1(COc2ccc3c(Nc4cnc(Cl)c(C(=O)O)c4)nccc3c2)COC1]

To a solution of methyl 2-chloro-5-((6-((3-methyloxetan-3-yl)methoxy)isoquinolin-1-yl)amino)nicotinate (0.130 g, 0.314 mmol) in tetrahydrofuran (3 mL) and water (3 mL) was added lithium hydroxide (0.023 g, 0.960 mmol). The reaction mixture was stirred at ambient temperature for 1 h and then the pH was adjusted to pH 7 with 1 M hydrochloric acid. The reaction mixture was diluted with water (10 mL) and ethyl acetate (10 mL). The aqueous phase was extracted with a mixture of ethyl acetate and methanol (5:1, 2×15 mL). The combined organic phase was dried over anhydrous sodium sulfate, filtered, and the filtrate was concentrated in vacuo. The obtained residue was purified by silica gel column chromatography, eluting with 25% of methanol in ethyl acetate, to provide the title compound as a colorless solid (0.090 g, 72% yield): ¹H NMR (400 MHz, DMSO-d₆) δ9.46 (s, 1H), 8.99 (s, 1H), 8.74-8.63 (m, 1H), 8.48 (d, J=9.2 Hz, 1H), 7.99 (d, J=5.6 Hz, 1H), 7.71-7.51 (m, 1H), 7.42-7.30 (m, 2H), 4.55 (d, J=5.6 Hz, 2H), 4.35 (d, J=6.0 Hz, 2H), 4.23 (s, 2H), 1.42 (s, 3H), COOH not observed.

### Step 5. Preparation of tert-butyl (2-chloro-5-((6-((3-methyloxetan-3-yl)methoxy)isoquinolin-1-yl)amino)pyridin-3-yl)carbamate

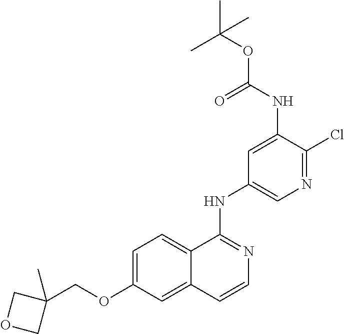 [SMILES: CC1(COc2ccc3c(Nc4cnc(Cl)c(NC(=O)OC(C)(C)C)c4)nccc3c2)COC1]

To a solution of 2-chloro-5-((6-((3-methyloxetan-3-yl)methoxy)isoquinolin-1-yl)amino)nicotinic acid (0.090 g, 0.225 mmol) in toluene (2 mL) was added diphenylphosphoryl azide (0.100 mL, 0.463 mmol), triethylamine (0.050 mL, 0.359 mmol), and 2-methylpropan-2-ol (0.250 mL, 2.61 mmol). The mixture was stirred at 110° C. for 12 h. After cooling to ambient temperature, water (10 mL) and ethyl acetate (10 mL) were added to the reaction mixture. The mixture was extracted with ethyl acetate (2×15 mL). The combined organic phase was washed with brine (30 mL), dried over anhydrous sodium sulfate, and filtered. The filtrate was concentrated in vacuo and the obtained residue was purified by silica gel column chromatography, eluting with 50% of ethyl acetate in petroleum ether, to provide a colorless solid (0.050 g, 47% yield): MS (ES+) m/z 471.2 (M+1), 473.2 (M+1).

### Step 6. Preparation of 2-chloro-N⁵-(6-((3-methyloxetan-3-yl)methoxy)isoquinolin-1-yl)pyridine-3,5-diamine

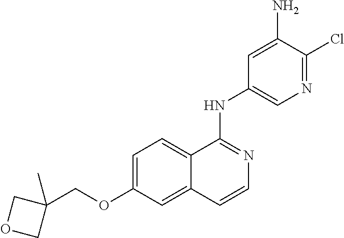 [SMILES: CC1(COc2ccc3c(Nc4cnc(Cl)c(N)c4)nccc3c2)COC1]

To a solution of tert-butyl (2-chloro-5-((6-((3-methyloxetan-3-yl)methoxy)isoquinolin-1-yl)amino)pyridin-3-yl)carbamate (0.05 g, 0.106 mmol) in dichloromethane (5 mL) was added trifluoroacetic acid (1.0 mL, 13.5 mmol) and the mixture was stirred at ambient temperature for 3 h. The reaction mixture was diluted with water (10 mL) and extracted with ethyl acetate (3×10 mL). The combined organic phase was washed with brine (30 mL), dried over anhydrous sodium sulfate, and filtered. The filtrate was concentrated in vacuo and the obtained residue was purified by silica gel column chromatography, eluting with 50% of ethyl acetate in petroleum ether. The residue was then purified by reverse-phase preparative HPLC (Phenomenex Luna C18 150 mm×25 mm, 10 μm column), eluting with a gradient of 8% to 28% of acetonitrile in water (containing 0.5% of formic acid), to provide the title compound as a colorless solid (0.014 g, 19% yield): ¹H-NMR (400 MHz, DMSO-d₆) δ 9.10 (s, 1H), 8.45 (d, J=9.2 Hz, 1H), 8.03 (d, J=2.4 Hz, 1H), 7.95 (d, J=5.6 Hz, 1H), 7.87 (d, J=2.4 Hz, 1H), 7.37-7.22 (m, 2H), 7.14 (d, J=5.6 Hz, 1H), 5.46 (s, 2H), 4.55 (d, J=5.6 Hz, 2H), 4.35 (d, J=6.0 Hz, 2H), 4.22 (s, 2H), 1.41 (s, 3H); MS (ES+) m/z 371.3 (M+1), 373.1 (M+1).

### Example 164

### Synthesis of N-(6-chloropyridin-3-yl)-6-((1,5-dimethyl-1H-pyrazol-4-yl)methoxy)isoquinolin-1-amine

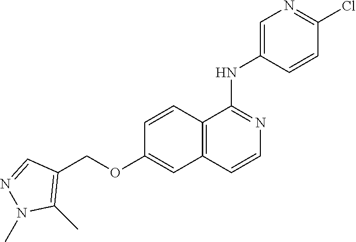 [SMILES: Cc1c(COc2ccc3c(Nc4ccc(Cl)nc4)nccc3c2)cnn1C]

### Step 1: Preparation of 1-chloro-6-((1,5-dimethyl-1H-pyrazol-4-yl)methoxy)isoquinoline

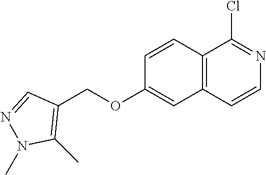 [SMILES: Cc1c(COc2ccc3c(Cl)nccc3c2)cnn1C]

To a solution of (1,5-dimethylpyrazol-4-yl)methanol (0.076 g, 0.606 mmol) in N,N-dimethylformamide (5.5 mL) was added sodium hydride (60% dispersion in mineral oil, 0.026 g, 0.661 mmol) and the resulting mixture was stirred for 20 minutes at ambient temperature. To the reaction mixture was then added 1-chloro-6-fluoroisoquinoline (0.063 g, 0.148 mmol), and the reaction mixture was stirred at ambient temperature for 16 h. The mixture was quenched by addition of water (10 mL). After dilution with ethyl acetate (20 mL), the mixture was washed with saturated ammonium chloride (20 mL) and brine (20 mL). The organic phase was dried over anhydrous sodium sulfate, filtered, and the filtrate was concentrated in vacuo. The obtained residue was purified by silica gel column chromatography, eluting with a gradient of 0 to 100% of ethyl acetate in heptane, to afford the title compound as a colorless oil (0.083 g, 52% yield): ¹H NMR (400 MHz, CDCl₃) δ8.24-8.19 (m, 2H), 7.53 (s, 1H), 7.49 (d, J=5.8 Hz, 1H), 7.30 (dd, J=9.3, 2.4 Hz, 1H), 7.20 (d, J=2.3 Hz, 1H), 5.02 (s, 2H), 3.82 (s, 3H), 2.32 (s, 3H); MS (ES+) m/z 288.5 (M+1), 290.5 (M+1).

### Step 2: Preparation of N-(6-chloropyridin-3-yl)-6-((1,5-dimethyl-1H-pyrazol-4-yl)methoxy)isoquinolin-1-amine

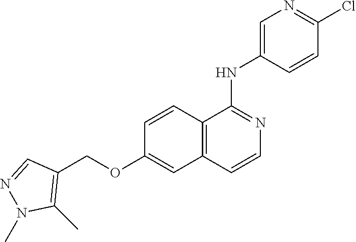 [SMILES: Cc1c(COc2ccc3c(Nc4ccc(Cl)nc4)nccc3c2)cnn1C]

To a solution of 1-chloro-6-((1,5-dimethyl-1H-pyrazol-4-yl)methoxy)isoquinoline (0.083 g, 0.289 mmol) in 1,4-dioxane (5 mL) was added 5-amino-2-chloropyridine (0.041 g, 0.318 mmol), tris(dibenzylideneacetone)dipalladium(0) (0.026 g, 0.029 mmol), 2-dicyclohexylphosphino-2′,6′-dimethoxy-1,1′-biphenyl (0.024 g, 0.058 mmol) and potassium phosphate tribasic (0.245 g, 1.16 mmol). The mixture was degassed by passing a stream of nitrogen through it for 5 minutes, and then the reaction mixture was heated to 100° C. for 2 h. After cooling to ambient temperature, the reaction mixture was filtered through celite, and the filtrate was concentrated in vacuo. The residue was purified by silica gel column chromatography, eluting with a gradient of 0 to 100% of ethyl acetate in heptane, to provide the title compound as a colorless solid (0.040 g, 36% yield): ¹H NMR (400 MHz, CDCl₃) δ 8.50 (d, J=2.8 Hz, 1H), 8.35 (dd, J=8.7, 2.7 Hz, 1H), 8.02 (d, J=5.8 Hz, 1H), 7.87 (d, J=9.1 Hz, 1H), 7.53 (s, 1H), 7.30 (d, J=8.7 Hz, 1H), 7.22-7.17 (m, 2H), 7.12 (d, J=5.9 Hz, 1H), 5.01 (s, 2H), 3.81 (s, 3H), 2.32 (s, 3H), NH not observed; MS (ES+) m/z 380.2 (M+1), 382.2 (M+1).

### Examples 165-166

In a similar manner as described in EXAMPLE 164, utilizing the appropriately substituted starting materials and intermediates, the following compounds were prepared:

| | | MS | |
|---|---|---|---|
| | | (ES+) | |
| Compound No. | Name | m/z | NMR |
| 165 | N-(6-chloropyridin-3- | 380.2 | ¹H NMR (400 MHz, CDCl₃) δ 8.50 |
| | yl)-6-((1,3-dimethyl- | (M + 1), | (d, J = 2.8 Hz, 1H), 8.35 (dd, J = |
| | 1H-pyrazol-4- | 382.2 | 8.6, 2.9 Hz, 1H), 8.02 (d, J = 5.8 |
| | yl)methoxy)isoquinolin- | (M + 1) | Hz, 1H), 7.87 (d, J = 9.1 Hz, 1H), |
| | 1-amine | | 7.41 (s, 1H), 7.31 (d, J = 8.7 Hz, |
| | | | 1H), 7.21 (dd, J = 9.1, 2.5 Hz, |
| | | | 1H), 7.14 (dd, J = 12.2, 4.1 Hz, |
| | | | 2H), 5.02 (s, 2H), 3.85 (s, 3H), |
| | | | 2.32 (s, 3H), NH not observed. |
| 166 | N-(6-chloropyridin-3- | 396.0 | ¹H NMR (400 MHz, DMSO-d₆) δ |
| | yl)-6-((3-methoxy-1- | (M + 1), | 9.37 (s, 1H), 8.88 (d, J = 2.8 Hz, |
| | methyl-1H-pyrazol- | 398.0 | 1H), 8.43-8.40 (m, 2H), 7.96 (d, J = |
| | 4- | (M + 1) | 5.7 Hz, 1H), 7.72 (s, 1H), 7.44 |
| | yl)methoxy)isoquinolin- | | (d, J = 8.8 Hz, 1H), 7.39 (d, J = |
| | 1-amine | | 2.4 Hz, 1H), 7.24 (dd, J = 9.2, 2.5 |
| | | | Hz, 1H), 7.18 (d, J = 5.8 Hz, 1H), |
| | | | 4.93 (s, 2H), 3.83 (s, 3H), 3.69 (s, 3H). |

### Example 167

### Synthesis of N-((1s,4s)-4-((1-((6-chloropyridin-3-yl)amino)isoquinolin-6-yl)oxy)cyclohexyl)-4-methylthiazole-5-carboxamide

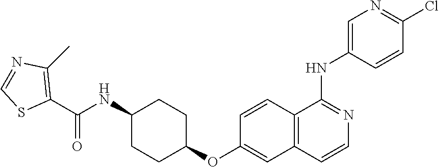 [SMILES: Cc1ncsc1C(=O)N[C@H]1CC[C@@H](Oc2ccc3c(Nc4ccc(Cl)nc4)nccc3c2)CC1]

### Step 1. Preparation of tert-butyl ((1s,4s)-4-((1-chloroisoquinolin-6-yl)oxy)cyclohexyl)carbamate

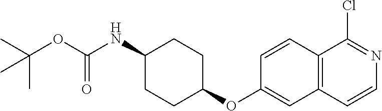 [SMILES: CC(C)(C)OC(=O)N[C@H]1CC[C@@H](Oc2ccc3c(Cl)nccc3c2)CC1]

Following the procedure as described or EXAMPLE 164, Step 1, and making variations as required to replace (1,5-dimethylpyrazol-4-yl)methanol with tert-butyl cis-4-hydroxycyclohexylcarbamate, the title compound was obtained as a colorless oil (0.213 g, 51% yield): ¹H NMR (400 MHz, CDCl₃) δ 8.21 (d, J=9.2 Hz, 1H), 8.15 (d, J=5.7 Hz, 1H), 7.43 (d, J=5.7 Hz, 1H), 7.26 (dd, J=9.2, 2.4 Hz, 1H), 7.06 (d, J=2.4 Hz, 1H), 4.65 (s, 1H), 4.58-4.56 (m, 1H), 3.62-3.56 (m, 1H), 2.09-2.05 (m, 2H), 1.84-1.72 (m, 4H), 1.62 (dd, J=10.5, 2.4 Hz, 2H), 1.44 (s, 9H); MS (ES+) m/z 377.8 (M+1), 379.8 (M+1).

### Step 2. Preparation of N-((1s,4s)-4-((1-chloroisoquinolin-6-yl)oxy)cyclohexyl)-4-methylthiazole-5-carboxamide

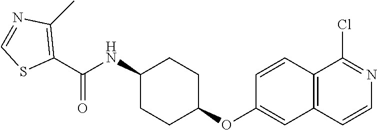 [SMILES: Cc1ncsc1C(=O)N[C@H]1CC[C@@H](Oc2ccc3c(Cl)nccc3c2)CC1]

To a solution of tert-butyl ((1s,4s)-4-((1-chloroisoquinolin-6-yl)oxy)cyclohexyl)carbamate (0.213 g, 0.564 mmol) in dichloromethane (5.6 mL) was added trifluoroacetic acid (0.86 mL, 11.3 mmol) and the reaction mixture was stirred at ambient temperature for 16 h. The mixture was concentrated in vacuo and co-distilled with toluene (3×10 mL). The obtained residue was dissolved in dichloromethane (5.6 mL), followed by addition of 4-methyl-5-thiazolecarboxylic acid (0.0970 g, 0.677 mmol), benzotriazol-1-yloxytripyrrolidinophosphonium hexafluorophosphate (0.352 g, 0.677 mmol), and N,N-diisopropylethylamine (0.29 mL, 1.69 mmol). The reaction mixture was stirred at ambient temperature for 2 h and then diluted with methanol (5 mL). The resulting mixture was stirred at ambient temperature for 30 minutes and then diluted with saturated sodium bicarbonate solution (30 mL). The mixture was extracted with dichloromethane (3×30 mL). The combined organic phase was washed with brine (20 mL), dried over anhydrous sodium sulfate, and filtered. The filtrate was concentrated in vacuo, and the obtained residue was purified by silica gel column chromatography, eluting with a gradient of 0 to 60% of ethyl acetate in heptane, to provide the title compound as a colorless solid (0.201 g, 88% yield): ¹H NMR (400 MHz, CDCl₃) δ 8.74 (s, 1H), 8.26 (d, J=9.2 Hz, 1H), 8.20 (d, J=5.7 Hz, 1H), 7.49 (d, J=5.9 Hz, 1H), 7.33 (dd, J=9.3, 2.5 Hz, 1H), 7.13 (d, J=2.4 Hz, 1H), 5.79 (d, J=7.8 Hz, 1H), 3.72 (td, J=6.6, 4.2 Hz, 1H), 3.42-3.38 (m, 1H), 3.18 (dt, J=7.4, 3.7 Hz, 2H), 2.76 (s, 2H), 2.21-2.16 (m, 2H), 2.00-1.96 (m, 2H), 1.89-1.82 (m, 2H), NH not observed; MS (ES+) m/z 402.0 (M+1), 404.0 (M+1).

### Step 3. Preparation of N-((1s,4s)-4-((1-((6-chloropyridin-3-yl)amino)isoquinolin-6-yl)oxy)cyclohexyl)-4-methylthiazole-5-carboxamide

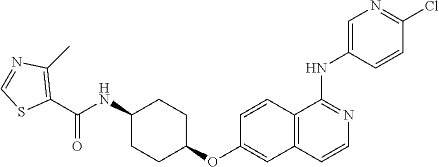 [SMILES: Cc1ncsc1C(=O)N[C@H]1CC[C@@H](Oc2ccc3c(Nc4ccc(Cl)nc4)nccc3c2)CC1]

Following the procedure as described for EXAMPLE 164, Step 2, and making variations as required to replace 1-chloro-6-((1,5-dimethyl-1H-pyrazol-4-yl)methoxy)isoquinoline with N-((1s,4s)-4-((1-chloroisoquinolin-6-yl)oxy)cyclohexyl)-4-methylthiazole-5-carboxamide, the title compound was obtained as a yellow solid (0.005 g, 2% yield): ¹H NMR (500 MHz, CDCl₃) δ8.71 (s, 1H), 8.49 (d, J=2.7 Hz, 1H), 8.32 (dd, J=8.7, 2.8 Hz, 1H), 7.98 (d, J=5.8 Hz, 1H), 7.91 (d, J=9.1 Hz, 1H), 7.29 (d, J=8.7 Hz, 1H), 7.20 (d, J=2.4 Hz, 1H), 7.09-7.07 (m, 2H), 5.79 (d, J=7.8 Hz, 1H), 4.71 (s, 1H), 4.12-4.07 (m, 1H), 2.74 (s, 3H), 2.17-2.14 (m, 2H), 1.96-1.93 (m, 2H), 1.82-1.75 (m, 4H), NH not observed; MS (ES+) m/z 494.2 (M+1), 496.0 (M+1).

### Example 168

### Synthesis of 6-((5-(1H-1,2,4-triazol-1-yl)pentyl)oxy)-N-(6-chloropyridin-3-yl)isoquinolin-1-amine

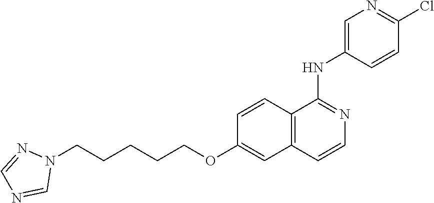 [SMILES: Clc1ccc(Nc2nccc3cc(OCCCCCn4cncn4)ccc23)cn1]

### Step 1. Preparation of ethyl 5-((1-chloroisoquinolin-6-yl)oxy)pentanoate

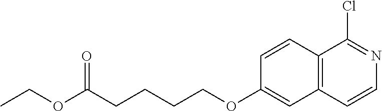 [SMILES: CCOC(=O)CCCCOc1ccc2c(Cl)nccc2c1]

Following the procedure as described for EXAMPLE 36, Step 2 and making variations as required to replace (3-methyloxetan-3-yl)methyl 4-methylbenzenesulfonate with ethyl 5-bromopentanoate, the title compound was obtained as a colorless oil (0.380 g, quantitative yield): ¹H NMR (400 MHz, CDCl₃) δ 8.21 (dd, J=9.3, 3.2 Hz, 1H), 8.17 (dd, J=5.7, 2.7 Hz, 1H), 7.46 (dd, J=5.7, 2.4 Hz, 1H), 7.29-7.26 (m, 1H), 7.06 (t, J=2.5 Hz, 1H), 4.15-4.10 (m, 4H), 2.42 (t, J=6.5 Hz, 2H), 1.93-1.84 (m, 4H), 1.26 (t, J=7.1 Hz, 3H); MS (ES+) m/z 308.4 (M+1), 310.4 (M+1).

### Step 2. Preparation of 5-((1-chloroisoquinolin-6-yl)oxy)pentan-1-ol

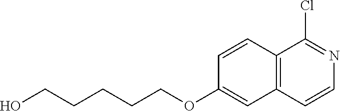 [SMILES: OCCCCCOc1ccc2c(Cl)nccc2c1]

To a solution of ethyl 5-((1-chloroisoquinolin-6-yl)oxy)pentanoate (0.380 g, 1.16 mmol) in tetrahydrofuran (5.8 mL) was added lithium borohydride (4 M in tetrahydrofuran, 0.87 mL, 3.48 mmol) and methanol (0.38 mL, 3.48 mmol) at 0° C. The resulting mixture was allowed to warm up to the ambient temperature and stirred for 5 h. The reaction mixture was cooled to 0° C. and quenched by addition of saturated ammonium chloride solution (20 mL). The mixture was extracted with ethyl acetate (3×20 mL), and the combined organic layers were dried over anhydrous sodium sulfate and filtered. The filtrate was concentrated in vacuo and the obtained residue was purified by silica gel column chromatography, eluting with a gradient of 0 to 60% of ethyl acetate in heptane, to provide the title compound as a colorless oil (0.212 g, 69% yield): MS (ES+) m/z 266.5 (M+1), 268.5 (M+1).

### Step 3. Preparation of 5-((1-chloroisoquinolin-6-yl)oxy)pentyl 4-methylbenzenesulfonate

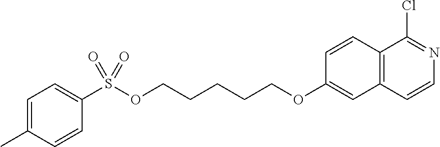 [SMILES: Cc1ccc(S(=O)(=O)OCCCCCOc2ccc3c(Cl)nccc3c2)cc1]

Following the procedure as described for EXAMPLE 36, Step 1 and making variations as required to replace 3-methyl-3-oxetanemethanol with 5-((1-chloroisoquinolin-6-yl)oxy)pentan-1-01, the title compound was obtained as a colorless solid (0.146 g, 62% yield): MS (ES+) m/z 420.0 (M+1), 422.0 (M+1).

### Step 4. Preparation of 6-((5-(1H-1,2,4-triazol-1-yl)pentyl)oxy)-1-chloroisoquinoline

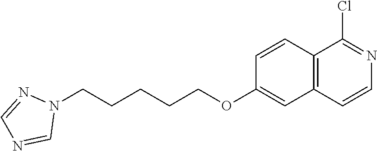 [SMILES: Clc1nccc2cc(OCCCCCn3cncn3)ccc12]

To the solution of 5-((1-chloroisoquinolin-6-yl)oxy)pentyl 4-methylbenzenesulfonate (0.146 g, 0.347 mmol) in acetonitrile (3 mL) was added 1,2,4-triazole (0.020 g, 0.290 mmol) and potassium carbonate (0.048 g, 0.347 mmol) and the reaction mixture was heated to 80° C. for 4 h. After cooling to ambient temperature, the reaction mixture was diluted with saturated sodium bicarbonate solution (20 mL) and extracted with ethyl acetate (3×20 mL). The combined organic layers were dried over anhydrous sodium sulfate and filtered, and the filtrate was concentrated in vacuo. The obtained residue was purified by silica gel column chromatography, eluting with a gradient of 0 to 100% of ethyl acetate in heptane, to provide the title compound as a colorless oil (0.049 g, 53% yield): MS (ES+) m/z 317.5 (M+1), 319.5 (M+1).

### Step 5. Preparation of 6-((5-(1H-1,2,4-triazol-1-yl)pentyl)oxy)-N-(6-chloropyridin-3-yl)isoquinolin-1-amine

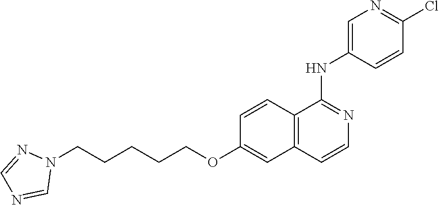 [SMILES: Clc1ccc(Nc2nccc3cc(OCCCCCn4cncn4)ccc23)cn1]

Following the procedure as described for EXAMPLE 164, Step 2 and making variations as required to replace 1-chloro-6-((1,5-dimethyl-1H-pyrazol-4-yl)methoxy)isoquinoline with 6-((5-(1H-1,2,4-triazol-1-yl)pentyl)oxy)-1-chloroisoquinoline, the title compound was obtained as a yellow solid (0.020 g, 31% yield): ¹H NMR (400 MHz, CDCl₃) δ 8.49 (d, J=2.7 Hz, 1H), 8.29 (dd, J=8.7, 2.8 Hz, 1H), 8.09 (s, 1H), 7.98 (d, J=5.8 Hz, 1H), 7.96 (s, 1H), 7.88-7.85 (m, 1H), 7.30 (d, J=8.6 Hz, 1H), 7.17-7.14 (m, 1H), 7.11-7.09 (m, 1H), 7.02-7.02 (m, 1H), 4.23 (t, J=7.0 Hz, 2H), 4.09 (t, J=6.2 Hz, 2H), 2.03-1.98 (m, 2H), 1.92-1.87 (m, 2H), 1.58-1.50 (m, 2H), NH not observed; MS (ES+) m/z 409.0 (M+1), 411.0 (M+1).

### Example 169

### Synthesis of 6-(1-(1-methyl-1H-pyrazol-4-yl)ethoxy)-N-(2-methylpyrimidin-5-yl)isoquinolin-1-amine

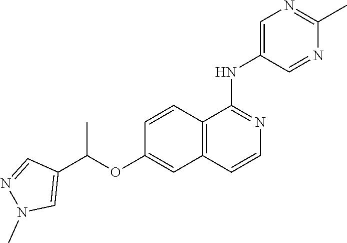 [SMILES: Cc1ncc(Nc2nccc3cc(OC(C)c4cnn(C)c4)ccc23)cn1]

### Step 1. Preparation of 1-chloro-6-(1-(1-methyl-1H-pyrazol-4-yl)ethoxy)isoquinoline

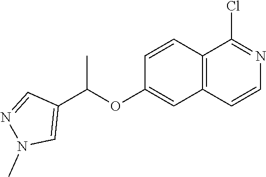 [SMILES: CC(Oc1ccc2c(Cl)nccc2c1)c1cnn(C)c1]

Following the procedure as described for EXAMPLE 164, Step 1, and making variations as required to replace (1,5-dimethylpyrazol-4-yl)methanol with 1-(1-methylpyrazol-4-yl)ethanol, the title compound was obtained as a colorless solid (0.399 g, 63% yield): MS (ES+) m/z 288.2 (M+1), 290.2 (M+1).

### Step 2. Preparation of 6-(1-(1-methyl-1H-pyrazol-4-yl)ethoxy)-N-(2-methylpyrimidin-5-yl)isoquinolin-1-amine

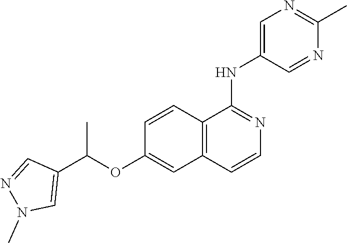 [SMILES: Cc1ncc(Nc2nccc3cc(OC(C)c4cnn(C)c4)ccc23)cn1]

To a solution of 1-chloro-6-(1-(1-methyl-1H-pyrazol-4-yl)ethoxy)isoquinoline (0.633 g, 2.20 mmol) in 1,4-dioxane (11 mL) was added 2-methylpyrimidin-5-amine (0.204 g, 1.87 mmol)), tris(dibenzylideneacetone)dipalladium(0) (0.210 g, 0.220 mmol), 2-dicyclohexylphosphino-2′,6′-dimethoxy-1,1′-biphenyl (0.181 g, 0.440 mmol), and potassium phosphate tribasic (1.87 g, 8.80 mmol). The mixture was degassed by passing a stream of nitrogen through it for 5 minutes, and then heated to 100° C. for 4 h. After cooling to ambient temperature, the reaction mixture was filtered through celite, and the filtrate was concentrated in vacuo. The residue was purified by silica gel column chromatography, eluting with a gradient of 0 to 100% of ethyl acetate in heptane, to provide the title compound as a colorless solid (0.298 g, 37% yield): ¹H NMR (400 MHz, CDCl₃) δ 9.00 (s, 2H), 8.12 (s, 1H), 7.95 (d, J=5.8 Hz, 1H), 7.50 (s, 1H), 7.35 (s, 1H), 7.16 (dd, J=9.1, 2.3 Hz, 1H), 7.04 (dd, J=11.1, 4.0 Hz, 2H), 5.55 (q, J=6.3 Hz, 1H), 3.84 (s, 3H), 2.68 (s, 3H), 1.69 (d, J=6.4 Hz, 3H), NH not observed; MS (ES+) m/z 361.2 (M+1).

### Example 170

### Synthesis of N-(6-chloropyridin-3-yl)-6-(1-(1-methyl-1H-pyrazol-4-yl)ethoxy)isoquinolin-1-amine

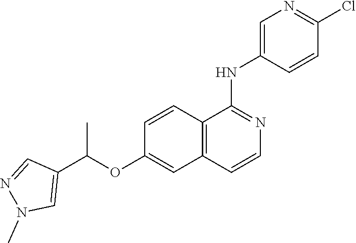 [SMILES: CC(Oc1ccc2c(Nc3ccc(Cl)nc3)nccc2c1)c1cnn(C)c1]

### Step 1. Preparation of 1-chloro-6-(1-(1-methyl-1H-pyrazol-4-yl)ethoxy)isoquinoline

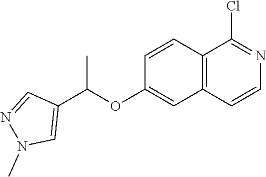 [SMILES: CC(Oc1ccc2c(Cl)nccc2c1)c1cnn(C)c1]

Following the procedure as described for EXAMPLE 164, Step 1, and making variations as required to replace (1,5-dimethylpyrazol-4-yl)methanol with 1-(1-methyl-1H-pyrazol-4-yl)ethan-1-ol, the title compound was obtained as a colorless oil (0.100 g, 12% yield): MS (ES+) m/z 288.5 (M+1), 290.5 (M+1).

### Step 2. Preparation of N-(6-chloropyridin-3-yl)-6-(1-(1-methyl-1H-pyrazol-4-yl)ethoxy)isoquinolin-1-amine

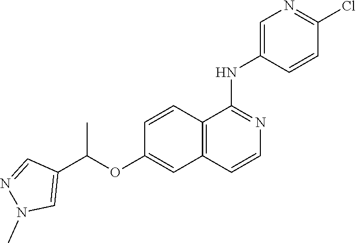 [SMILES: CC(Oc1ccc2c(Nc3ccc(Cl)nc3)nccc2c1)c1cnn(C)c1]

Following the procedure as described for EXAMPLE 164, Step 2, and making variations as required to replace 1-chloro-6-((1,5-dimethyl-1H-pyrazol-4-yl)methoxy)isoquinoline with 1-chloro-6-(1-(1-methyl-1H-pyrazol-4-yl)ethoxy)isoquinoline, the title compound was obtained as a yellow solid (0.083 g, 63% yield): ¹H NMR (400 MHz, CDCl₃) δ 8.48 (d, J=2.9 Hz, 1H), 8.34 (dd, J=8.7, 2.9 Hz, 1H), 7.99 (d, J=5.8 Hz, 1H), 7.84 (d, J=9.1 Hz, 1H), 7.51 (s, 1H), 7.35 (s, 1H), 7.29 (d, J=8.7 Hz, 1H), 7.20 (dd, J=9.1, 2.5 Hz, 1H), 7.06 (dd, J=9.0, 4.2 Hz, 2H), 5.57 (q, J=6.4 Hz, 1H), 3.86 (s, 3H), 1.71 (d, J=6.4 Hz, 3H), NH not observed; MS (ES+) m/z 380.0 (M+1), 382.0 (M+1).

### Examples 171 and 172

### Synthesis of (R)—N-(6-chloropyridin-3-yl)-6-(1-(1-methyl-1H-pyrazol-4-yl)ethoxy)isoquinolin-1-amine and (S)—N-(6-chloropyridin-3-yl)-6-(1-(1-methyl-1H-pyrazol-4-yl)ethoxy)isoquinolin-1-amine

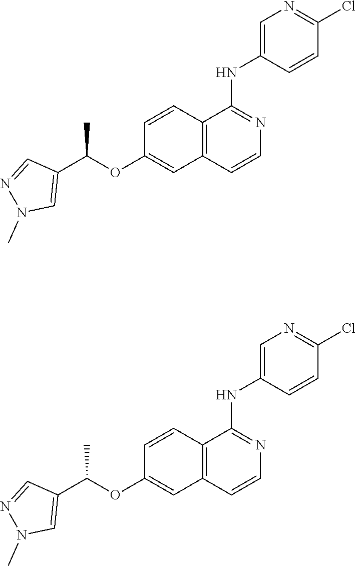 [SMILES: C[C@@H](Oc1ccc2c(Nc3ccc(Cl)nc3)nccc2c1)c1cnn(C)c1 ; C[C@H](Oc1ccc2c(Nc3ccc(Cl)nc3)nccc2c1)c1cnn(C)c1]

Racemic N-(6-chloropyridin-3-yl)-6-(1-(1-methyl-1H-pyrazol-4-yl)ethoxy)isoquinolin-1-amine was synthesized as described in EXAMPLE 170. Resolution of the enantiomers by chiral SFC (ChiralCel OJ 150×4.6 mm, 5 μm column), eluting with a gradient of 5 to 60% of isopropanol (containing 10 mM of ammonium formate) in supercritical carbon dioxide, afforded the title compounds as single enantiomers as colorless solids. First eluting enantiomer (0.027 g, 34% yield): ¹H NMR (400 MHz, CDCl₃) δ 8.48 (d, J=2.9 Hz, 1H), 8.36-8.33 (m, 1H), 7.99 (d, J=5.8 Hz, 1H), 7.84 (d, J=9.1 Hz, 1H), 7.51 (s, 1H), 7.36 (s, 1H), 7.30 (d, J=8.7 Hz, 1H), 7.20 (dd, J=9.1, 2.4 Hz, 1H), 7.07 (dd, J=8.3, 4.1 Hz, 2H), 5.59-5.55 (m, 1H), 3.86 (s, 3H), 1.71 (d, J=6.3 Hz, 3H), NH not observed; MS (ES+) m/z 380.0 (M+1), 382.2 (M+1). Second eluting enantiomer (0.026 g, 33% yield): ¹H NMR (400 MHz, CDCl₃) δ8.50 (d, J=2.6 Hz, 1H), 8.36 (dd, J=8.7, 2.8 Hz, 1H), 8.02-8.00 (m, 1H), 7.86 (d, J=9.1 Hz, 1H), 7.54 (s, 1H), 7.38 (s, 1H), 7.32 (d, J=8.6 Hz, 1H), 7.24-7.21 (m, 1H), 7.09 (dd, J=7.9, 4.0 Hz, 2H), 5.62-5.57 (m, 1H), 3.89 (s, 3H), 1.73 (d, J=6.4 Hz, 3H), NH not observed; MS (ES+) m/z 380.0 (M+1), 382.2 (M+1).

### Example 173

### Synthesis of N-(6-chloropyridin-3-yl)-6-(1-(1,3-dimethyl-1H-pyrazol-4-yl)ethoxy)isoquinolin-1-amine

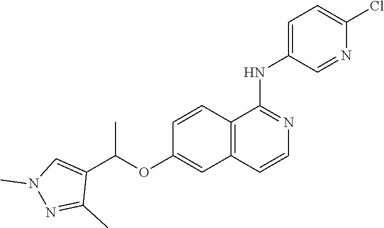 [SMILES: Cc1nn(C)cc1C(C)Oc1ccc2c(Nc3ccc(Cl)nc3)nccc2c1]

### Step 1. Preparation of 1-chloro-6-(1-(1,3-dimethyl-1H-pyrazol-4-yl)ethoxy)isoquinoline

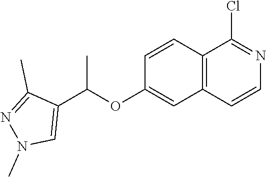 [SMILES: Cc1nn(C)cc1C(C)Oc1ccc2c(Cl)nccc2c1]

Following the procedure as described for EXAMPLE 164, Step 1, and making variations as required to replace (1,5-dimethylpyrazol-4-yl)methanol with 1-(1,3-dimethyl-1H-pyrazol-4-yl)ethan-1-ol, the title compound was obtained as a colorless oil (0.214 g, 34% yield): MS (ES+) m/z 302.5 (M+1), 304.5 (M+1).

### Step 2. Preparation of N-(6-chloropyridin-3-yl)-6-(1-(1,3-dimethyl-1H-pyrazol-4-yl)ethoxy)isoquinolin-1-amine

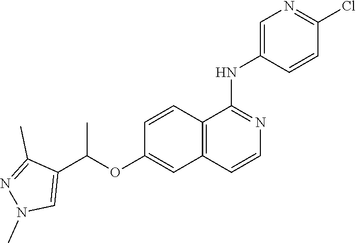 [SMILES: Cc1nn(C)cc1C(C)Oc1ccc2c(Nc3ccc(Cl)nc3)nccc2c1]

Following the procedure as described for EXAMPLE 164, Step 2, and making variations as required to replace 1-chloro-6-((1,5-dimethyl-1H-pyrazol-4-yl)methoxy)isoquinoline with 1-chloro-6-(1-(1,3-dimethyl-1H-pyrazol-4-yl)ethoxy)isoquinoline, the title compound was obtained as a yellow solid (0.096 g, 31% yield): ¹H NMR (400 MHz, CDCl₃) δ 8.48 (d, J=2.6 Hz, 1H), 8.35-8.32 (m, 1H), 7.98-7.97 (m, 1H), 7.85 (d, J=9.1 Hz, 1H), 7.29 (d, J=8.2 Hz, 2H), 7.18 (dd, J=9.1, 2.0 Hz, 1H), 7.03 (dd, J=9.6, 3.9 Hz, 2H), 5.48 (q, J=6.3 Hz, 1H), 3.78 (s, 3H), 2.30 (s, 3H), 1.68 (d, J=6.4 Hz, 3H), NH not observed; MS (ES+) m/z 394.2 (M+1), 396.2 (M+1).

### Examples 174 and 175

### Synthesis of (R)—N-(6-chloropyridin-3-yl)-6-(1-(1,3-dimethyl-1H-pyrazol-4-yl)ethoxy)isoquinolin-1-amine and (S)—N-(6-chloropyridin-3-yl)-6-(1-(1,3-dimethyl-1H-pyrazol-4-yl)ethoxy)isoquinolin-1-amine

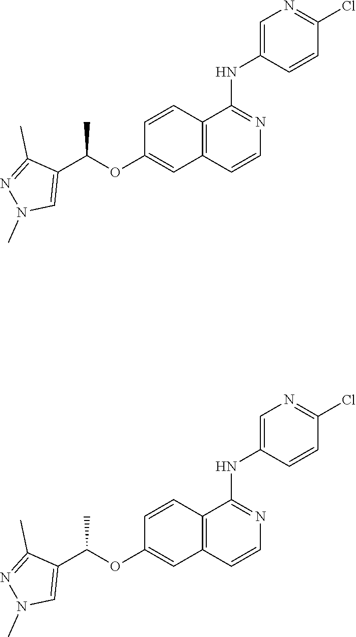 [SMILES: Cc1nn(C)cc1[C@@H](C)Oc1ccc2c(Nc3ccc(Cl)nc3)nccc2c1 ; Cc1nn(C)cc1[C@H](C)Oc1ccc2c(Nc3ccc(Cl)nc3)nccc2c1]

Racemic N-(6-chloropyridin-3-yl)-6-(1-(1,3-dimethyl-1H-pyrazol-4-yl)ethoxy)isoquinolin-1-amine was synthesized as described in EXAMPLE 173. Resolution of the enantiomers by chiral (ChiralPak IC 150×4.6 mm, 5 μm column), eluting with eluting with a gradient of 5 to 60% of methanol (containing 10 mM of ammonium formate) in supercritical carbon dioxide, afforded the title compounds as single enantiomers as colorless solids. First eluting enantiomer (0.030 g, 38% yield): ¹H NMR (400 MHz, CDCl₃) δ 8.47 (d, J=2.8 Hz, 1H), 8.34 (dd, J=8.7, 2.8 Hz, 1H), 7.98 (d, J=5.8 Hz, 1H), 7.83 (d, J=9.2 Hz, 1H), 7.29 (d, J=8.4 Hz, 2H), 7.18 (dd, J=9.1, 2.5 Hz, 1H), 7.04 (d, J=5.9 Hz, 1H), 7.02 (d, J=2.4 Hz, 1H), 5.48 (q, J=6.4 Hz, 1H), 3.78 (s, 3H), 2.30 (s, 3H), 1.68 (d, J=6.4 Hz, 3H), NH not observed; MS (ES+) m/z 394.2 (M+1), 396.2 (M+1). Second eluting enantiomer (0.029 g, 36% yield): ¹H NMR (400 MHz, CDCl₃) δ 8.47 (d, J=2.8 Hz, 1H), 8.34 (dd, J=8.7, 2.8 Hz, 1H), 7.98 (d, J=5.8 Hz, 1H), 7.83 (d, J=9.1 Hz, 1H), 7.29 (d, J=8.4 Hz, 2H), 7.19-7.17 (m, 1H), 7.05 (s, 1H), 7.02 (d, J=2.3 Hz, 1H), 5.48 (q, J=6.3 Hz, 1H), 3.78 (s, 3H), 2.30 (s, 3H), 1.68 (d, J=6.4 Hz, 3H), NH not observed; MS (ES+) m/z 394.2 (M+1), 396.2 (M+1).

### Example 176

### Synthesis of 6-(2-amino-2,3-dimethylbutoxy)-N-(6-chloropyridin-3-yl)isoquinolin-1-amine hydrochloride

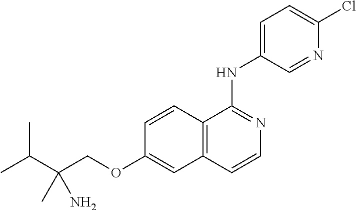 [SMILES: CC(C)C(C)(N)COc1ccc2c(Nc3ccc(Cl)nc3)nccc2c1]

### Step 1: Preparation of 2-amino-2,3-dimethylbutan-1-ol

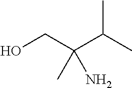

To a solution of 2-amino-2,3-dimethylbutanoic acid (1.50 g, 11.4 mmol) in tetrahydrofuran (15 mL) was add lithium aluminum hydride (0.868 g, 22.9 mmol) at 0° C. The reaction mixture was then heated to 70° C. for 4 h. After cooling to ambient temperature, the mixture was quenched by addition of a mixture of sodium sulfate decahydrate and celite (1:1 by weight) until gas evolution ceased. The reaction mixture was then filtered, and the filter cake was washed with tetrahydrofuran (20 mL) and methanol (20 mL). The combined filtrate was concentrated in vacuo to give the title compound as colorless oil (1.30 g, 87% yield): ¹H NMR (400 MHz, DMSO-d₆) δ 4.73-4.13 (m, 1H), 3.69-3.20 (m, 2H), 3.14 (d, J=2.4 Hz, 2H), 1.63-1.53 (m, 1H), 0.86-0.76 (m, 9H).

### Step 2: Preparation of tert-butyl (1-hydroxy-2,3-dimethylbutan-2-yl)carbamate

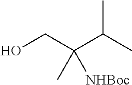

To a mixture of 2-amino-2,3-dimethylbutan-1-ol (0.500 g, 4.27 mmol) and triethylamine (0.475 g, 4.69 mmol) in dichloromethane (10 mL) was added di-tert-butyl dicarbonate (1.02 g, 4.69 mmol) and the reaction mixture was stirred at ambient temperature for 12 h. After concentration of the mixture in vacuo, the obtained residue was purified by silica gel column chromatography, eluting with a gradient of 0 to 20% of ethyl acetate in petroleum ether, to provide the title compound as colorless oil. (0.590 g, 60% yield): ¹H NMR (400 MHz, DMSO-d₆) δ 6.06 (s, 1H), 4.65 (t, J=5.2 Hz, 1H), 3.51-3.44 (m, 1H), 3.38 (d, J=5.2 Hz, 1H), 2.27-2.11 (m, 1H), 1.36 (s, 9H), 1.00 (s, 3H), 0.83-0.79 (m, 6H).

### Step 3: Preparation of tert-butyl 4-isopropyl-4-methyl-1,2,3-oxathiazolidine-3-carboxylate 2,2-dioxide

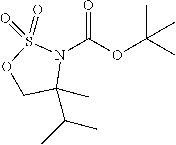 [SMILES: CC(C)C1(C)COS(=O)(=O)N1C(=O)OC(C)(C)C]

To a solution of sulfurous dichloride (0.737 g, 6.19 mmol) in dichloromethane (20 mL) was added tert-butyl (1-hydroxy-2,3-dimethylbutan-2-yl)carbamate (1.10 g, 5.06 mmol) in dichloromethane (10 mL) at −20° C. The mixture was stirred at −20° C. for 10 minutes, then pyridine (1.61 g, 20.3 mmol) was added to it. The reaction mixture was stirred at −20° C. for 1 h, and then at 0° C. for 1 h. The mixture was diluted with dichloromethane (20 mL) and washed with 1 M hydrochloric acid (20 mL) and saturated sodium bicarbonate solution (20 mL). The organic phase was dried over anhydrous sodium sulfate and filtered. The filtrate was concentrated in vacuo to give a colorless oil (1.30 g). To the oil was added acetonitrile (10 mL), followed by addition of ruthenium(III) chloride (0.0520 g, 0.251 mmol), sodium periodate (1.58 g, 7.40 mmol), and water (10 mL) at 0° C. The mixture was stirred at 0° C. for 1 h, and then diluted with ethyl acetate (50 mL). The mixture was washed with saturated disodium sulfite solution (3×50 mL). The organic phase was dried over anhydrous sodium sulfate and filtered. The filtrate was concentrated under in vacuo. The obtained residue was purified by silica gel column chromatography, eluting with a gradient of 0 to 10% of ethyl acetate in petroleum ether, to provide the title compound as a colorless solid (1.30 g, 90% yield): ¹H NMR (400 MHz, DMSO-d₆) d 4.63 (d, J=10.0 Hz, 1H), 4.37 (d, J=10.0 Hz, 1H), 2.34-2.37 (m, 1H), 1.50 (s, 3H), 1.47 (s, 9H), 0.94-0.86 (m, 6H).

### Step 5: Preparation of tert-butyl (1-((1-chloroisoquinolin-6-yl)oxy)-2,3-dimethylbutan-2-yl)carbamate

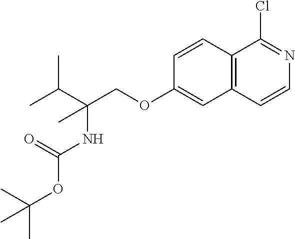 [SMILES: CC(C)C(C)(COc1ccc2c(Cl)nccc2c1)NC(=O)OC(C)(C)C]

To a solution of tert-butyl 4-isopropyl-4-methyl-1,2,3-oxathiazolidine-3-carboxylate 2,2-dioxide (0.778 g, 2.79 mmol) and 1-chloroisoquinolin-6-ol (0.500 g, 2.78 mmol) in N,N-dimethylformamide (10 mL) was added potassium carbonate (1.17 g, 8.44 mmol) and the reaction mixture was heated to 100° C. for 12 h. After cooling to ambient temperature, the reaction mixture was diluted with ethyl acetate (20 mL) and washed with brine (3×20 mL). The organic phase was dried over anhydrous sodium sulfate and filtered. The filtrate was concentrated in vacuo and the obtained residue was purified by silica gel column chromatography, eluting with a gradient of 0 to 30% of ethyl acetate in petroleum ether, to provide the title compound as colorless oil (0.500 g, 45% yield): ¹H NMR (400 MHz, DMSO-d₆) δ 8.19 (d, J=5.6 Hz, 1H), 8.15 (d, J=9.2 Hz, 1H), 7.77 (d, J=5.6 Hz, 1H), 7.51 (d, J=2.4 Hz, 1H), 7.41 (dd, J=9.2, 2.4 Hz, 1H), 6.52 (s, 1H), 4.41 (d, J=9.2 Hz, 1H), 4.18 (d, J=9.2 Hz, 1H), 2.42-2.29 (m, 1H), 1.29 (s, 9H), 1.19 (s, 3H), 0.90 (dd, J=6.8, 1.6 Hz, 6H).

### Step 6: Preparation of tert-butyl (1-((1-((6-chloropyridin-3-yl)amino)isoquinolin-6-yl)oxy)-2,3-dimethylbutan-2-yl)carbamatebutyl (2,3-dimethyl-1-((1-((2-methylpyrimidin-5-yl)amino)isoquinolin-6-yl)oxy)butan-2-yl)carbamate

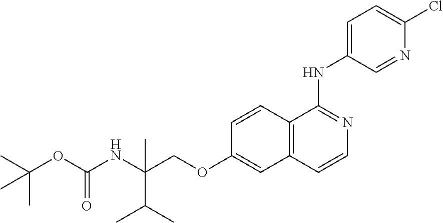 [SMILES: CC(C)C(C)(COc1ccc2c(Nc3ccc(Cl)nc3)nccc2c1)NC(=O)OC(C)(C)C]

To a solution of tert-butyl (1-((1-chloroisoquinolin-6-yl)oxy)-2,3-dimethylbutan-2-yl)carbamate (0.100 g, 0.264 mmol), 6-chloropyridin-3-amine (0.0410 g, 0.319 mmol), and cesium carbonate (0.258 g, 0.792 mmol) in 2-methylbutan-2-ol (2 mL) was added [(2-di-tert-butylphosphino-2′,4′,6′-triisopropyl-1,1′-biphenyl)-2-(2′-amino-1,1′-biphenyl)]palladium(II) methanesulfonate (0.0210 g, 0.0264 mmol) and the reaction mixture was heated to 70° C. for 12 h. After cooling to ambient temperature, the mixture was concentrated under reduced pressure. The obtained residue was purified by silica gel column chromatography, eluting with a gradient of 0 to 100% of ethyl acetate in petroleum ether, and then purified by reverse-phase preparative HPLC, eluing with a gradient of 38-68% of acetonitrile in water containing 0.1% of formic acid, to provide the title compound as a colorless solid (0.050 g, 38% yield): ¹H NMR (400 MHz, DMSO-d₆) δ 9.36 (s, 1H), 8.87 (d, J=2.8 Hz, 1H), 8.42 (dd, J=8.8, 2.8 Hz, 2H), 7.95 (d, J=5.6 Hz, 1H), 7.44 (d, J=8.8 Hz, 1H), 7.30 (d, J=2.4 Hz, 1H), 7.26 (dd, J=9.2, 2.4 Hz, 1H), 7.19 (d, J=6.0 Hz, 1H), 6.48 (s, 1H), 4.37 (d, J=9.6 Hz, 1H), 4.14 (d, J=9.6 Hz, 1H), 2.42-2.25 (m, 1H), 1.31 (s, 9H), 1.20 (s, 3H), 0.90 (dd, J=6.8, 2.0 Hz, 6H).

### Step 7: Preparation of 6-(2-amino-2,3-dimethylbutoxy)-N-(6-chloropyridin-3-yl)isoquinolin-1-amine hydrochloride

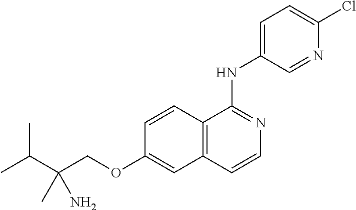 [SMILES: CC(C)C(C)(N)COc1ccc2c(Nc3ccc(Cl)nc3)nccc2c1]

To a solution of tert-butyl (1-((1-((6-chloropyridin-3-yl)amino)isoquinolin-6-yl)oxy)-2,3-dimethylbutan-2-yl)carbamate (0.050 g, 0.106 mmol) in dichloromethane (1 mL) was added 4 M hydrogen chloride in ethyl acetate (1.43 mL, 5.72 mmol) and the reaction mixture was stirred at ambient temperature for 1 h. The mixture was then concentrated under reduced pressure to afford the title compound as a colorless solid (0.032 g, 69% yield): ¹H NMR (400 MHz, DMSO-d₆) δ 8.84-8.68 (m, 2H), 8.38 (s, 3H), 8.25-8.15 (m, 1H), 7.80-7.71 (m, 1H), 7.65 (d, J=8.4 Hz, 1H), 7.56 (s, 1H), 7.48 (d, J=9.2 Hz, 1H), 7.30 (d, J=6.8 Hz, 1H), 4.28 (s, 2H), 2.28-2.13 (m, 1H), 1.28 (s, 3H), 1.03 (d, J=6.8 Hz, 3H), 0.97 (d, J=7.2 Hz, 3H); MS (ES+) m/z 371.2 (M+1), 373.2 (M+1).

### Example 177

### Synthesis of N-(6-chloropyridin-3-yl)-6-((1-methoxycyclopropyl)methoxy)isoquinolin-1-amine

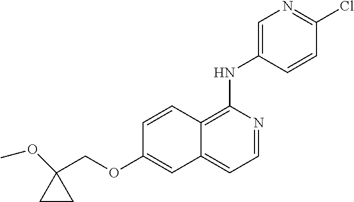 [SMILES: COC1(COc2ccc3c(Nc4ccc(Cl)nc4)nccc3c2)CC1]

### Step 1: Preparation of 1-chloro-6-((1-methoxycyclopropyl)methoxy)isoquinoline

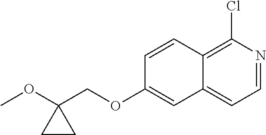 [SMILES: COC1(COc2ccc3c(Cl)nccc3c2)CC1]

To a solution of 1-chloroisoquinolin-6-ol (0.200 g, 1.11 mmol) and (1-methoxycyclopropyl)methanol (0.148 g, 1.45 mmol) in tetrahydrofuran (10 mL) was added triphenylphosphine (0.438 g, 1.67 mmol) followed by diisopropyl azodicarboxylate (0.338 g, 1.67 mmol) at 0° C. The reaction was allowed to warm to ambient temperature and stirred for 12 h. To the mixture was then added saturated ammonium chloride solution (20 mL) and the mixture was extracted with ethyl acetate (3×20 mL). The combined organic layers were washed with brine (3×20 mL), dried over anhydrous sodium sulfate, and concentrated in vacuo. The obtained residue was purified by silica gel column chromatography, eluting with a gradient of 9 to 33% of ethyl acetate in petroleum ether, to afford the title compound as a colorless solid (0.600 g, 82% yield): ¹H NMR (400 MHz, CDCl₃) δ 8.25 (d, J=9.2 Hz, 1H), 8.20 (d, J=6.0 Hz, 1H), 7.48 (d, J=5.6 Hz, 1H), 7.38 (dd, J=9.2, 2.4 Hz, 1H), 7.09 (d, J=2.4 Hz, 1H), 4.22 (s, 2H), 3.45 (s, 3H), 1.09-1.00 (m, 2H), 0.83-0.75 (m, 2H).

### Step 2. Preparation of N-(6-chloropyridin-3-yl)-6-((1-methoxycyclopropyl)methoxy)isoquinolin-1-amine

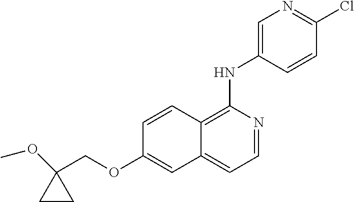 [SMILES: COC1(COc2ccc3c(Nc4ccc(Cl)nc4)nccc3c2)CC1]

To a solution of 11-chloro-6-((1-methoxycyclopropyl)methoxy)isoquinoline (0.030 g, 0.114 mmol) and 6-chloropyridin-3-amine (0.018 g, 0.137 mmol) in 2-methylbutan-2-ol (2 mL) was added cesium carbonate (0.074 g, 0.228 mmol) followed by methanesulfonato(2-dicyclohexylphosphino-2′,6′-di-i-propoxy-1,1′-biphenyl)(2′-amino-1,1′-biphenyl-2-yl)palladium(II) (0.001 g, 0.011 mmol). The reaction was heated to 100° C. for 2 h in a microwave reactor. After cooling to ambient temperature, the mixture was diluted with water (20 mL) and extracted with ethyl acetate (3×20 mL). The combined organic layers were washed with brine (3×20 mL), dried over anhydrous sodium sulfate, and concentrated in vacuo. The obtained residue was purified by silica gel column chromatography, eluting with a gradient of 17 to 50% of ethyl acetate in petroleum ether, and then by reverse-phase preparative HPLC, eluting with a gradient of 46 to 76% of acetonitrile in water containing 0.1% of ammonium hydroxide, to provide the title compound as a colorless solid (0.011 g, 27% yield): ¹H NMR (400 MHz, CDCl₃) δ 8.52 (d, J=2.8 Hz, 1H), 8.36 (dd, J=8.8, 2.8 Hz, 1H), 8.03 (d, J=5.6 Hz, 1H), 7.88 (d, J=9.6 Hz, 1H), 7.32 (d, J=9.2 Hz, 1H), 7.30-7.27 (m, 1H), 7.11 (d, J=5.6 Hz, 2H), 7.07 (d, J=2.4 Hz, 1H), 4.22 (s, 2H), 3.46 (s, 3H), 1.11-0.99 (m, 2H), 0.84-0.74 (m, 2H); MS (ES+) m/z 356.1 (M+1), 358.1 (M+1).

### Examples 178-187

In a similar manner as described in EXAMPLE 177, utilizing the appropriately substituted starting materials and intermediates, the following compounds were prepared:

| | | MS | |
|---|---|---|---|
| | | (ES+) | |
| Compound No. | Name | m/z | NMR |
| 178 | 6-((1- | 337.2 | ¹H NMR (400 MHz, CDCl₃) δ 9.01 (s, |
| | methoxycyclopropyl)methoxy)- | (M + 1). | 2H), 8.01-7.94 (m, 1H), 7.91-7.83 |
| | N-(2- | | (m, 1H), 7.31-7.28 (m, 1H), 7.27- |
| | methylpyrimidin-5- | | 7.24 (m, 1H), 7.13 (d, J = 5.6 Hz, 1H), |
| | yl)isoquinolin-1-amine | | 7.08 (d, J = 1.6 Hz, 1H), 4.22 (s, 2H), |
| | | | 3.45 (s, 3H), 2.74 (s, 3H), 1.07-1.00 |
| | | | (m, 2H), 0.83-0.73 (m, 2H). |
| 179 | N-(6-chloropyridin-3- | 340.1 | ¹H NMR (400 MHz, DMSO-d₆) δ 9.37 |
| | yl)-6-((1- | (M + 1), | (s, 1H), 8.88 (d, J = 2.8 Hz, 1H), |
| | methylcyclopropyl)methoxy)isoquinolin- | 342.1 | 8.45-8.41 (m, 2H), 7.95 (d, J = 5.6 |
| | 1- | (M + 1). | Hz, 1H), 7.44 (d, J = 8.8 Hz, 1H), 7.30 |
| | amine | | (dd, J = 9.2, 2.4 Hz, 1H), 7.23 (d, J = |
| | | | 2.4 Hz, 1H), 7.15 (d, J = 6.0 Hz, 1H), |
| | | | 3.92 (s, 2H), 1.22 (s, 3H), 0.59-0.56 |
| | | | (m, 2H), 0.45-0.42 (m, 2H). |
| 180 | N-(6-chloropyridin-3- | 339.9 | ¹H NMR (400 MHz, CDCl₃) δ 8.51 (d, |
| | yl)-6-(2- | (M + 1), | J = 2.4 Hz, 1H), 8.36 (dd, J = 8.8, 2.8 |
| | cyclopropylethoxy)isoquinolin- | 341.9 | Hz, 1H), 8.02 (d, J = 5.8 Hz, 1H), 7.85 |
| | 1-amine | (M + 1). | (d, J = 9.2 Hz, 1H), 7.32 (d, J = 8.4 |
| | | | Hz, 1H), 7.22 (dd, J = 9.2, 2.4 Hz, |
| | | | 1H), 7.14-7.07 (m, 2H), 4.19 (t, J = |
| | | | 6.4 Hz, 2H), 1.78 (q, J = 6.4 Hz, 2H), |
| | | | 0.97-0.84 (m, 1H), 0.58-0.50 (m, |
| | | | 2H), 0.21-0.14 (m, 2H), NH not |
| | | | observed. |
| 181 | N-(6-chloropyridin-3- | 344.2 | ¹H NMR (400 MHz, DMSO-d₆) δ 9.39 |
| | yl)-6-((1- | (M + 1), | (s, 1H), 8.89 (d, J = 2.4 Hz, 1H), 8.46 |
| | fluorocyclopropyl)methoxy)isoquinolin- | 346.2 | (d, J = 9.3 Hz, 1H), 8.43 (dd, J = 8.7, |
| | 1- | (M + 1). | 2.9 Hz, 1H), 7.97 (d, J = 5.8 Hz, 1H), |
| | amine | | 7.45 (d, J = 8.7 Hz, 1H), 7.36 (dd, J = |
| | | | 9.2, 2.6 Hz, 1H), 7.32 (d, J = 2.6 Hz, |
| | | | 1H), 7.17 (d, J = 5.8 Hz, 1H), 4.47 (d, |
| | | | J = 22.6 Hz, 2H), 1.22-1.13 (m, 2H), |
| | | | 0.98-0.90 (m, 2H); ¹⁹F NMR (376 |
| | | | MHz, DMSO-d₆) δ −185.3 (s). |
| 182 | N-(6-chloropyridin-3- | 363.9 | ¹H NMR (400 MHz, DMSO-d₆) δ 9.41 |
| | yl)-6-(pyrimidin-5- | (M + 1), | (s, 1H), 9.22 (s, 1H), 9.01 (s, 2H), |
| | ylmethoxy)isoquinolin- | 371.9 | 8.89 (d, J = 2.5 Hz, 1H), 8.48 (d, J = |
| | 1-amine | (M + 1). | 9.0 Hz, 1H), 8.43 (dd, J = 8.7, 2.7 Hz, |
| | | | 1H), 7.99 (d, J = 5.7 Hz, 1H), 7.50- |
| | | | 7.43 (m, 2H), 7.39 (dd, J = 9.3, 2.0 |
| | | | Hz, 1H), 7.22 (d, J = 5.8 Hz, 1H), 5.36 |
| | | | (s, 2H). |
| 183 | N-(6-chloropyridin-3- | 377.2 | ¹H NMR (400 MHz, CDCl₃) δ 8.59 (d, |
| | yl)-6-(2-(pyridin-2- | (M + 1), | J = 4.4 Hz, 1H), 8.48 (s, 1H), 8.34 (d, |
| | yl)ethoxy)isoquinolin-1- | 379.2 | J = 8.3 Hz, 1H), 7.99 (d, J = 5.8 Hz, |
| | amine | (M + 1). | 1H), 7.83 (d, J = 9.0 Hz, 1H), 7.66 (t, |
| | | | J = 7.5 Hz, 1H), 7.30 (d, J = 8.1 Hz, |
| | | | 2H), 7.20-7.15 (m, 3H), 7.09 (d, J = |
| | | | 2.6 Hz, 2H), 4.52 (t, J = 6.7 Hz, 2H), |
| | | | 3.35 (t, J = 6.6 Hz, 2H). |
| 184 | N-(6-chloropyridin-3- | 384.2 | ¹H NMR (400 MHz, DMSO-d₆) δ 9.41 |
| | yl)-6-(2- | (M + 1), | (s, 1H), 8.88 (d, J = 2.8 Hz, 1H), 8.46 |
| | (trifluoromethoxy)ethoxy)isoquinolin- | 386.0 | (d, J = 10.0 Hz, 1H), 8.42 (dd, J = 8.8, |
| | 1-amine | (M + 1). | 2.9 Hz, 1H), 7.98 (d, J = 5.8 Hz, 1H), |
| | | | 7.45 (d, J = 8.7 Hz, 1H), 7.34-7.32 (m, |
| | | | 2H), 7.19 (d, J = 5.8 Hz, 1H), 4.50 (t, |
| | | | J = 3.9 Hz, 2H), 4.43-4.41 (m, 2H); |
| | | | ¹⁹F NMR (376 MHz, DMSO-d₆) δ −58.9 (s). |
| 185 | 6-(3-(1H-imidazol-1- | 380.1 | ¹H NMR (400 MHz, DMSO-d₆) δ 9.37 |
| | yl)propoxy)-N-(6- | (M + 1), | (s, 1H), 8.88 (d, J = 2.8 Hz, 1H), |
| | chloropyridin-3- | 382.1 | 8.57-8.29 (m, 2H), 7.96 (d, J = 5.6 |
| | yl)isoquinolin-1-amine | (M + 1). | Hz, 1H), 7.65 (s, 1H), 7.45 (d, J = 8.8 |
| | | | Hz, 1H), 7.33-7.21 (m, 3H), 7.18 (d, J = |
| | | | 5.6 Hz, 1H), 6.91 (s, 1H), 4.19 (t, J = |
| | | | 7.2 Hz, 2H), 4.08 (t, J = 6.0 Hz, 2H), |
| | | | 2.28-2.24 (m, 2H). |
| 186 | N-(6-chloropyridin-3- | 394.2 | ¹H NMR (400 MHz, DMSO-d₆) δ 9.40 |
| | yl)-6-((2- | (M + 1), | (s, 1H), 8.88 (d, J = 2.8 Hz, 1H), 8.81 |
| | methoxypyrimidin-5- | 396.2 | (s, 2H), 8.50-8.45 (m, 1H), 8.42 (dd, J = |
| | yl)methoxy)isoquinolin- | (M + 1). | 8.8, 2.9 Hz, 1H), 7.98 (d, J = 5.8 Hz, |
| | 1-amine | | 1H), 7.45 (dd, J = 5.6, 3.0 Hz, 2H), |
| | | | 7.36-7.33 (m, 1H), 7.22 (d, J = 5.9 Hz, |
| | | | 1H), 5.25 (s, 2H), 3.94 (s, 3H). |
| 187 | N-(5-methoxy-6- | 366.2 | ¹H NMR (400 MHz, DMSO-d₆) 8 |
| | methylpyridin-3-yl)-6- | (M + 1), | 9.34-9.22 (m, 1H), 8.59-8.57 (m, 1H), |
| | ((3-methyloxetan-3- | 368.2 | 8.50-8.46 (m, 1H), 8.13 (s, 0.5H), |
| | yl)methoxy)isoquinolin- | (M + 1). | 7.98-7.91 (m, 2H), 7.35-7.30 (m, 2H), |
| | 1-amine formate | | 7.14 (d, J = 5.7 Hz, 1H), 4.55 (d, J = |
| | | | 5.8 Hz, 2H), 4.35 (d, J = 5.8 Hz, 2H), |
| | | | 4.23 (s, 2H), 3.83 (s, 3H), 2.33 (s, |
| | | | 3H), 1.42 (s, 3H), COOH not |
| | | | observed. |

### Example 188

### Synthesis of N-(6-chloropyridin-3-yl)-6-((1-ethyl-1H-pyrazol-3-yl)methoxy)isoquinolin-1-amine formate

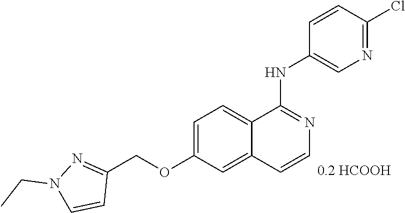 [SMILES: CCn1ccc(COc2ccc3c(Nc4ccc(Cl)nc4)nccc3c2)n1]

### Step 1. Preparation of 1-chloro-6-((1-ethyl-1H-pyrazol-3-yl)methoxy)isoquinoline

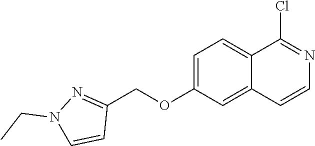 [SMILES: CCn1ccc(COc2ccc3c(Cl)nccc3c2)n1]

To a solution of 3-(chloromethyl)-1-ethyl-1H-pyrazole (0.242 g, 1.67 mmol) in N,N-dimethylformamide (3 mL) was added potassium carbonate (0.462 g, 3.34 mmol) and 1-chloroisoquinolin-6-ol (0.200 g, 1.11 mmol) at ambient temperature. The reaction mixture was heated up to 90° C. for 12 h. After cooling to ambient temperature, the reaction mixture was concentrated in vacuo and poured into water (30 mL). The mixture was extracted with ethyl acetate (3×15 mL). The combined organic layers were dried over anhydrous sodium sulfate, filtered, and the filtrate was concentrated in vacuo. The residue was purified by silica gel column chromatography, eluting with a gradient of 0 to 33% of ethyl acetate in petroleum ether, to provide the title compound as a yellowish solid (0.320 g, 77% yield): MS (ES+) m/z 288.2 (M+1), 290.2 (M+1).

### Step 2. Preparation of N-(6-chloropyridin-3-yl)-6-((1-ethyl-1H-pyrazol-3-yl)methoxy)isoquinolin-1-amine formate

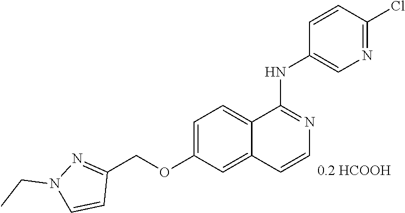 [SMILES: CCn1ccc(COc2ccc3c(Nc4ccc(Cl)nc4)nccc3c2)n1]

A mixture of 1-chloro-6-((1-ethyl-1H-pyrazol-3-yl)methoxy)isoquinoline (0.0500 g, 0.174 mmol), 6-chloropyridin-3-amine (0.0246 g, 0.191 mmol), [(2-di-tert-butylphosphino-2′,4′,6′-triisopropyl-1,1′-biphenyl)-2-(2′-amino-1,1′-biphenyl)] palladium(II) methanesulfonate (0.0014 g, 0.0017 mmol), and cesium carbonate (0.170 g, 0.521 mmol) in 2-methylbutan-2-ol (6 mL) was stirred at 90° C. for 12 h. After cooling to ambient temperature, the reaction mixture was concentrated in vacuo. The residue was poured into water (15 mL). The mixture was extracted with ethyl acetate (3×15 mL). The combined organic layers were dried over anhydrous sodium sulfate, filtered, and the filtrate was concentrated in vacuo. The residue was purified by reverse-phase preparative HPLC (Shim-pack C18 150 mm×25 mm, 10 μm column), eluting with a gradient of 13% to 43% of acetonitrile in water containing 0.225% of formic acid, to provide the title compound as a colorless solid (0.0178 g, 24% yield): ¹H NMR (400 MHz, CDCl₃) δ8.47 (d, J=2.8 Hz, 1H), 8.29 (dd, J=8.7, 2.9 Hz, 1H), 8.13 (s, 0.2H), 7.99 (d, J=6.0 Hz, 1H), 7.84 (d, J=9.3 Hz, 1H), 7.41 (d, J=2.2 Hz, 1H), 7.30 (d, J=8.8 Hz, 1H), 7.28-7.25 (m, 1H), 7.23-7.22 (m, 1H), 7.13 (d, J=6.1 Hz, 1H), 6.37 (d, J=2.3 Hz, 1H), 5.22 (s, 2H), 4.23-4.18 (m, 2H), 1.51 (t, J=7.3 Hz, 3H), NH and COOH not observed; MS (ES+) m/z 380.2 (M+1), 382.2 (M+1).

### Example 189

### Synthesis of 6-((1-fluorocyclopropyl)methoxy)-N-(2-methylpyrimidin-5-yl)isoquinolin-1-amine

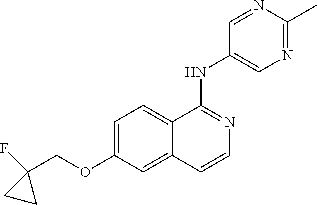 [SMILES: Cc1ncc(Nc2nccc3cc(OCC4(F)CC4)ccc23)cn1]

To a mixture of 1-chloro-6-((1-fluorocyclopropyl)methoxy)isoquinoline (0.500 g, 1.99 mmol) and 2-methylpyrimidin-5-amine (0.260 g, 2.38 mmol) in dioxane (30 mL) were added sodium carbonate (0.632 g, 5.96 mmol), and chloro(2-dicyclohexylphosphino-2′,6′-diisopropoxy-1,1′-biphenyl)[2-(2′-amino-1,1′-biphenyl)]palladium(II) (RuPhos Pd G2, 0.154 g, 0.199 mmol) and the mixture was heated at 90° C. for 12 h. After cooling to ambient temperature, the mixture was poured into water (30 mL) and extracted with ethyl acetate (3×50 mL). The combined organic phase was washed with brine (30 mL), dried over anhydrous sodium sulfate, and filtered. The filtrate was concentrated under reduced pressure and the obtained residue was purified by silica gel column chromatography, eluting with a gradient of 10-100% of ethyl acetate in petroleum ether. Further purification of the residue by reverse-phase preparative HPLC (Phenomenex Luna C18 150 mm×40 mm×15 μm column), eluting with 10-40% of acetonitrile in water (containing 0.225% of formic acid), provided the title compound as a colorless solid (0.550 g, 84% yield): ¹H NMR (400 MHz, DMSO-d₆) δ9.31 (s, 1H), 9.16 (s, 2H), 8.44 (d, J=9.2 Hz, 1H), 7.95 (d, J=5.6 Hz, 1H), 7.35 (dd, J=9.2, 2.6 Hz, 1H), 7.30 (d, J=2.4 Hz, 1H), 7.14 (d, J=5.6 Hz, 1H), 4.56-4.39 (m, 2H), 2.57 (s, 3H), 1.17 (dt, J=18.6, 6.8 Hz, 2H), 0.98-0.85 (m, 2H); ¹⁹F NMR (376 MHz, DMSO-d₆) δ−185.3 (s); MS (ES+) m/z 325.1 (M+1).

### Example 190

### Synthesis of 6-((1-fluorocyclopropyl)methoxy)-N-(6-methylpyridin-3-yl)isoquinolin-1-amine

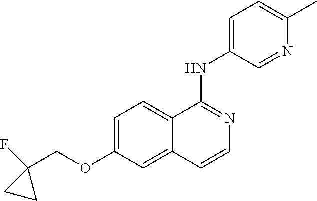 [SMILES: Cc1ccc(Nc2nccc3cc(OCC4(F)CC4)ccc23)cn1]

Following the procedure as described for EXAMPLE 189, and making variations as required to replace 2-methylpyrimidin-5-amine with 6-methylpyridin-3-amine, the title compound was obtained as a colorless solid (1.26 g, 89% yield): ¹H NMR (400 MHz, DMSO-d₆) δ9.14 (s, 1H), 8.83 (d, J=2.4 Hz, 1H), 8.45 (d, J=9.2 Hz, 1H), 8.19 (dd, J=2.4, 8.4 Hz, 1H), 7.91 (d, J=5.4 Hz, 1H), 7.35-7.24 (m, 2H), 7.18 (d, J=8.4 Hz, 1H), 7.08 (d, J=6.0 Hz, 1H), 4.52-4.40 (m, 2H), 2.42 (s, 3H), 1.17 (td, J=6.8, 13.6 Hz, 2H), 0.92 (q, J=7.8 Hz, 2H); ¹⁹F NMR (376 MHz, DMSO-d₆) δ−185.3 (s); MS (ES+) m/z 324.3 (M+1).

### Example 191

### Synthesis of N-(6-chloropyridin-3-yl)-6-(3-(methylsulfonyl)propoxy)isoquinolin-1-amine

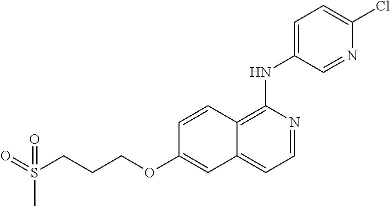 [SMILES: CS(=O)(=O)CCCOc1ccc2c(Nc3ccc(Cl)nc3)nccc2c1]

### Step 1. Preparation of N-(6-chloropyridin-3-yl)-6-(3-(methylthio)propoxy)isoquinolin-1-amine

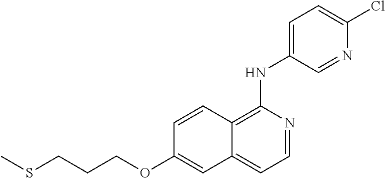 [SMILES: CSCCCOc1ccc2c(Nc3ccc(Cl)nc3)nccc2c1]

Following the procedure as described for EXAMPLE 177, and making variations as required to replace (1-methoxycyclopropyl)methanol with 3-(methylthio)propan-1-ol, the title compound was obtained as a colorless solid (0.080 g, 77% yield): ¹H NMR (400 MHz, CDCl₃) δ 8.51 (d, J=6.4 Hz, 1H), 8.38 (dd, J=8.8, 2.8 Hz, 1H), 8.03 (d, J=5.6 Hz, 1H), 7.85 (d, J=9.2 Hz, 1H), 7.32 (d, J=8.8 Hz, 1H), 7.21 (dd, J=9.2, 2.8 Hz, 1H), 7.12 (d, J=5.6 Hz, 1H), 7.09 (d, J=2.4 Hz, 1H), 7.03 (s, 1H), 4.23 (t, J=6.2 Hz, 2H), 2.75 (t, J=7.0 Hz, 2H), 2.21-2.14 (m, 5H).

### Step 1. Preparation of N-(6-chloropyridin-3-yl)-6-(3-(methylsulfonyl)propoxy)isoquinolin-1-amine

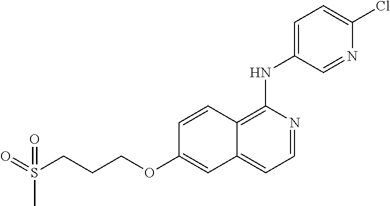 [SMILES: CS(=O)(=O)CCCOc1ccc2c(Nc3ccc(Cl)nc3)nccc2c1]

To a solution of N-(6-chloropyridin-3-yl)-6-(3-(methylthio)propoxy)isoquinolin-1-amine (0.028 g, 0.078 mmol) in dichloromethane (5 mL) was added 3-chloroperbenzoic acid (0.033 g, 0.163 mmol) at 0° C. The reaction was allowed to warm to ambient temperature and stirred for 16 h. To it was then added saturated sodium sulfite solution (10 mL) and the mixture was extracted with ethyl acetate (3×10 mL). The combined organic layers were washed with brine (3×10 mL), dried over anhydrous sodium sulfate and concentrated in vacuo. under vacuum. Purification of the obtained residue by reverse-phase preparative HPLC, eluting with a gradient of 30 to 60% of acetonitrile in water containing 0.1% of ammonium hydroxide, afforded the title compound as a light orange solid (0.012 g, 38% yield): ¹H NMR (400 MHz, DMSO-d₆) δ9.37 (s, 1H), 8.88 (d, J=2.8 Hz, 1H), 8.47-8.39 (m, 2H), 7.97 (d, J=5.6 Hz, 1H), 7.44 (d, J=8.8 Hz, 1H), 7.32-7.25 (m, 2H), 7.19 (d, J=6.4 Hz, 1H), 4.26 (t, J=6.4 Hz, 2H), 3.40-3.35 (m, 2H), 3.04 (s, 3H), 2.26-2.18 (m, 2H); MS (ES+) m/z 392.3 (M+1), 394.3 (M+1).

### Example 192

### Synthesis of N-(6-chloropyridin-3-yl)-6-(pyridin-4-ylmethoxy)isoquinolin-1-amine

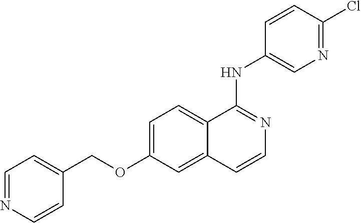 [SMILES: Clc1ccc(Nc2nccc3cc(OCc4ccncc4)ccc23)cn1]

### Step 1. Preparation of 1-chloro-6-(pyridin-4-ylmethoxy)isoquinoline

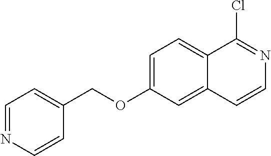 [SMILES: Clc1nccc2cc(OCc3ccncc3)ccc12]

To a mixture of 1-chloroisoquinolin-6-ol (0.100 g, 0.557 mmol) and 4-(chloromethyl)pyridine hydrochloride (0.091 g, 0.557 mmol) in N,N-dimethylformamide (1 mL) was added potassium carbonate (0.231 g, 1.67 mmol) and the reaction mixture was heated to 90° C. for 2 h. After cooling to ambient temperature, the reaction mixture was poured into water (30 mL). The mixture was extracted with ethyl acetate (3×30 mL). The combined organic layers were washed with brine (5×30 mL), dried over anhydrous sodium sulfate, and filtered. Concentration of the filtrate gave the title compound as a brownish solid (0.160 g, 99% yield): ¹H NMR (400 MHz, DMSO-d₆) δ8.64-8.58 (m, 2H), 8.24-8.18 (m, 2H), 7.78-7.72 (m, 1H), 7.58-7.48 (m, 4H), 5.42-5.36 (m, 2H).

### Step 2. Preparation of N-(6-chloropyridin-3-yl)-6-(pyridin-4-ylmethoxy)isoquinolin-1-amine

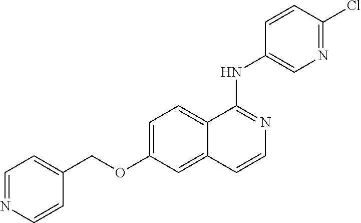 [SMILES: Clc1ccc(Nc2nccc3cc(OCc4ccncc4)ccc23)cn1]

To a mixture of 6-chloropyridin-3-amine (0.0617 g, 0.480 mmol), 1-chloro-6-(pyridin-4-ylmethoxy)isoquinoline (0.130 g, 0.480 mmol), and cesium carbonate (0.469 mg, 1.44 mmol) in 2-methylbutan-2-ol (1 mL) was added methanesulfonato (2-di-t-butylphosphino-2′,4′,6′-tri-i-propyl-1,1-biphenyl)(2′-amino-1,1-biphenyl-2-yl)palladium(II) (0.038 g, 0.048 mmol) and the reaction mixture was heated to 70° C. for 12 h. After cooling to ambient temperature, the mixture was purified by reverse-phase preparative HPLC, eluting with a gradient of 0 to 30% of acetonitrile in water containing 0.225% of formic acid, and then purified by reverse-phase preparative HPLC, eluting with a gradient of 29 to 59% of acetonitrile in water containing 0.05% of ammonium hydroxide, to give the title compound as an off-white solid (0.029 g, 16% yield): ¹H NMR (400 MHz, DMSO-d₆) δ 9.40 (s, 1H), 8.88 (d, J=2.8 Hz, 1H), 8.61 (d, J=5.8 Hz, 2H), 8.48 (d, J=9.0 Hz, 1H), 8.42 (dd, J=8.8, 2.8 Hz, 1H), 7.97 (d, J=5.8 Hz, 1H), 7.51 (d, J=5.8 Hz, 2H), 7.45 (d, J=8.8 Hz, 1H), 7.42-7.35 (m, 2H), 7.17 (d, J=5.8 Hz, 1H), 5.36 (s, 2H); MS (ES+) m/z 363.0 (M+1), 365.0 (M+1).

### Examples 193-206

In a similar manner as described in EXAMPLE 192, utilizing the appropriately substituted starting materials and intermediates, the following compounds were prepared:

| | | MS | |
|---|---|---|---|
| | | (ES+) | |
| Compound No. | Name | m/z | NMR |
| 193 | N-(6-chloropyridin-3- | 364.2 | ¹H NMR (400 MHz, CD₃OD) δ 8.87 (d, |
| | yl)-6-(pyrimidin-2- | (M + 1), | J = 5.0 Hz, 2H), 8.74 (d, J = 2.8 Hz, |
| | ylmethoxy)isoquinolin- | 366.3 | 1H), 8.34-8.25 (m, 2H), 7.92 (d, J = |
| | 1-amine | (M + 1). | 5.8 Hz, 1H), 7.50 (t, J = 4.9 Hz, 1H), |
| | | | 7.44-7.36 (m, 2H), 7.28 (d, J = 2.2 Hz, |
| | | | 1H), 7.15 (d, J = 5.8 Hz, 1H), 5.47 (s, |
| | | | 2H), NH not observed. |
| 194 | N-(6-chloropyridin-3- | 377.3 | ¹H NMR (400 MHz, CD₃OD) 88.74 (d, |
| | yl)-6-((6-methylpyridin- | (M + 1), | J = 2.7 Hz, 1H), 8.57 (d, J = 1.9 Hz, |
| | 3- | 379.3 | 1H), 8.32-8.26 (m, 2H), 7.93 (d, J = |
| | yl)methoxy)isoquinolin- | (M + 1). | 5.8 Hz, 1H), 7.90 (dd, J = 8.2, 2.1 Hz, |
| | 1-amine | | 1H), 7.44-7.37 (m, 2H), 7.35-7.29 (m, |
| | | | 2H), 7.20 (d, J = 6.0 Hz, 1H), 5.28 (s, |
| | | | 2H), 2.58 (s, 3H), NH not observed. |
| 195 | N-(6-chloropyridin-3- | 363.3 | ¹H NMR (400 MHz, DMSO-d₆) δ 9.39 |
| | yl)-6-(pyridin-3- | (M + 1), | (s, 1H), 8.89 (d, J = 2.8 Hz, 1H), 8.76 |
| | ylmethoxy)isoquinolin- | 365.3 | (d, J = 1.8 Hz, 1H), 8.59 (dd, J = 4.8, |
| | 1-amine | (M + 1). | 1.6 Hz, 1H), 8.47 (d, J = 9.3 Hz, 1H), |
| | | | 8.43 (dd, J = 8.7, 2.8 Hz, 1H), 8.01- |
| | | | 7.93 (m, 2H), 7.50-7.41 (m, 3H), 7.37 |
| | | | (dd, J = 9.2, 2.6 Hz, 1H), 7.20 (d, = |
| | | | 5.8 Hz, 1H), 5.33 (s, 2H). |
| 196 | N-(6-chloropyridin-3- | 366.1 | ¹H NMR (400 MHz, DMSO-d₆) δ 9.36 |
| | yl)-6-((1-methyl-1H- | (M + 1), | (s, 1H), 8.88 (d, J = 2.6 Hz, 1H), 8.42 |
| | pyrazol-4- | 368.1 | (dd, J = 8.8, 2.9 Hz, 2H), 7.97 (d, J = |
| | yl)methoxy)isoquinolin- | (M + 1). | 5.8 Hz, 1H), 7.86 (s, 1H), 7.58 (s, 1H), |
| | 1-amine | | 7.45 (d, J = 8.7 Hz, 1H), 7.40 (d, J = |
| | | | 2.6 Hz, 1H), 7.27 (dd, J = 9.2, 2.6 Hz, |
| | | | 1H), 7.20 (d, J = 5.8 Hz, 1H), 5.11 (s, |
| | | | 2H), 3.84 (s, 3H). |
| 197 | N-(6-chloropyridin-3- | 356.1 | ¹H NMR (400 MHz, DMSO-d₆) δ 9.38 |
| | yl)-6-((tetrahydro-2H- | (M + 1), | (s, 1H), 8.88 (d, J = 2.7 Hz, 1H), 8.46- |
| | pyran-4- | 358.1 | 8.40 (m, 2H), 7.95 (d, J = 5.8 Hz, 1H), |
| | yl)oxy)isoquinolin-1- | (M + 1). | 7.45 (d, J = 8.7 Hz, 1H), 7.38 (d, J = |
| | amine | | 2.5 Hz, 1H), 7.29 (dd, J = 9.2, 2.6 Hz, |
| | | | 1H), 7.18 (d, J = 5.8 Hz, 1H), 4.84- |
| | | | 4.77 (m, 1H), 3.94-3.86 (m, 2H), 3.60- |
| | | | 3.51 (m, 2H), 2.13-2.03 (m, 2H), 1.72- |
| | | | 1.59 (m, 2H). |
| 198 | N-(6-chloropyridin-3- | 366.2 | ¹H NMR (400 MHz, CD₃OD) 88.74 (d, |
| | yl)-6-((1-methyl-1H- | (M + 1), | J = 2.8 Hz, 1H), 8.33-8.25 (m, 2H), |
| | imidazol-2- | 368.2 | 7.94 (d, J = 5.8 Hz, 1H), 7.44-7.38 (m, |
| | yl)methoxy)isoquinolin- | (M + 1). | 2H), 7.30 (dd, J = 9.3, 2.4 Hz, 1H), |
| | 1-amine | | 7.21 (d, J = 5.9 Hz, 1H), 7.17 (d, J = |
| | | | 1.2 Hz, 1H), 7.00 (d, J = 1.3 Hz, 1H), |
| | | | 5.34 (s, 2H), 3.82 (s, 3H), NH not |
| | | | observed. |
| 199 | N-(2-methylpyrimidin-5- | 344.1 | ¹H NMR (400 MHz, DMSO-d₆) δ 9.33 |
| | yl)-6-(pyridin-4- | (M + 1). | (s, 1H), 9.16 (s, 2H), 8.62 (dd, J = 4.4, |
| | ylmethoxy)isoquinolin- | | 1.6 Hz, 2H), 8.47 (d, J = 9.2 Hz, 1H), |
| | 1-amine | | 7.96 (d, J = 5.8 Hz, 1H), 7.54-7.51 (m, |
| | | | 2H), 7.41 (dd, J = 9.1, 2.6 Hz, 1H), |
| | | | 7.37 (d, J = 2.5 Hz, 1H), 7.16 (d, J = |
| | | | 5.8 Hz, 1H), 5.36 (s, 2H), 2.58 (s, 3H). |
| 200 | N-(6-methylpyridin-3- | 343.2 | ¹H NMR (400 MHz, DMSO-d₆) δ 9.17 |
| | yl)-6-(pyridin-4- | (M + 1). | (br s, 1H), 8.84 (d, J = 2.0 Hz, 1H), |
| | ylmethoxy)isoquinolin- | | 8.61 (d, J = 4.8 Hz, 2H), 8.48 (d, J = |
| | 1-amine | | 8.8 Hz, 1H), 8.29-8.09 (m, 1H), 7.91 |
| | | | (d, J = 5.6 Hz, 1H), 7.50 (d, J = 5.2 |
| | | | Hz, 2H), 7.41-7.27 (m, 2H), 7.19 (d, J = |
| | | | 8.4 Hz, 1H), 7.09 (d, J = 5.6 Hz, |
| | | | 1H), 5.35 (s, 2H), 2.43 (s, 3H). |
| 201 | N-(6-chloropyridin-3- | 385.2 | ¹H NMR (400 MHz, CD₃OD3) 88.73 |
| | yl)-6-(2- | (M + 1), | (d, J = 2.6 Hz, 1H), 8.30-8.24 (m, 2H), |
| | morpholinoethoxy)isoquinolin- | 387.2 | 7.92 (d, J = 5.9 Hz, 1H), 7.40 (dd, J = |
| | 1-amine | (M + 1). | 8.7, 0.5 Hz, 1H), 7.28-7.22 (m, 2H), |
| | | | 7.19-7.17 (m, 1H), 4.31 (t, J = 5.5 Hz, |
| | | | 2H), 3.75 (t, J = 4.7 Hz, 4H), 2.90 (t, J = |
| | | | 5.4 Hz, 2H), 2.72-2.62 (m, 4H), NH |
| | | | not observed. |
| 202 | N-(2-chloropyrimidin-5- | 364.2 | ¹H NMR (400 MHz, DMSO-d₆) δ 9.63 |
| | yl)-6-(pyridin-4- | (M + 1). | (s, 1H), 9.30 (s, 2H), 8.62 (dd, J = 4.4, |
| | ylmethoxy)isoquinolin- | | 1.6 Hz, 2H), 8.48 (d, J = 9.2 Hz, 1H), |
| | 1-amine | | 8.01 (d, J = 5.8 Hz, 1H), 7.54-7.50 (m, |
| | | | 2H), 7.44 (dd, J = 9.2, 2.6 Hz, 1H), |
| | | | 7.41 (d, J = 2.5 Hz, 1H), 7.24 (d, J = |
| | | | 5.8 Hz, 1H), 5.37 (s, 2H). |
| 203 | N-(2-chloropyrimidin-5- | 367.0 | ¹H NMR (400 MHz, DMSO-d₆) δ 9.19 |
| | yl)-6-((1-methyl-1H- | (M + 1), | (s, 2H), 8.52-8.45 (m, 1H), 8.26 (d, J = |
| | pyrazol-3- | 369.0 | 9.2 Hz, 1H), 7.96 (d, J = 5.6 Hz, |
| | yl)methoxy)isoquinolin- | (M + 1). | 1H), 7.59 (d, J = 2.2 Hz, 1H), 7.34 (d, |
| | 1-amine | | J = 2.4 Hz, 1H), 7.28 (dd, J = 2.4, 9.2 |
| | | | Hz, 1H), 7.22 (d, J = 6.0 Hz, 1H), 6.41 |
| | | | (d, J = 2.2 Hz, 1H), 5.20 (s, 2H), 3.91 |
| | | | (s, 3H). |
| 204 | N-(6-chloropyridin-3- | 380.2 | ¹H NMR (400 MHz, CD₃OD) δ 8.73 (d, |
| | yl)-6-((1,4-dimethyl-1H- | (M + 1), | J = 2.7 Hz, 1H), 8.30-8.25 (m, 2H), |
| | pyrazol-3- | 382.2 | 7.92 (d, J = 5.9 Hz, 1H), 7.44-7.40 (m, |
| | yl)methoxy)isoquinolin- | (M + 1). | 2H), 7.37 (d, J = 2.5 Hz, 1H), 7.27 |
| | 1-amine | | (dd, J = 9.2, 2.6 Hz, 1H), 7.20 (d, J = |
| | | | 5.9 Hz, 1H), 5.19 (s, 2H), 3.86 (s, 3H), |
| | | | 2.14 (s, 3H), NH not observed. |
| 205 | 6-((1-methyl-1H- | 347.2 | ¹H NMR (400 MHz, CD₃OD) δ 9.15 |
| | pyrazol-4-yl)methoxy)- | (M + 1). | (s, 2H), 8.27 (d, J = 9.3 Hz, 1H), 7.94 |
| | N-(2-methylpyrimidin-5- | | (d, J = 5.9 Hz, 1H), 7.77 (s, 1H), 7.62 |
| | yl)isoquinolin-1-amine | | (s, 1H), 7.32 (d, J = 2.5 Hz, 1H), 7.25 |
| | | | (dd, J = 9.2, 2.6 Hz, 1H), 7.21 (d, J = |
| | | | 5.8 Hz, 1H), 5.16 (s, 2H), 3.92 (s, 3H), |
| | | | 2.68 (s, 3H), NH not observed. |
| 206 | 6-((1-methyl-1H- | 347.3 | ¹H NMR (400 MHz, CD₃OD) δ 9.12 |
| | pyrazol-3-yl)methoxy)- | (M + 1). | (s, 2H), 8.23 (d, J = 9.2 Hz, 1H), 7.91 |
| | N-(2-methylpyrimidin-5- | | (d, J = 6.0 Hz, 1H), 7.58 (d, J = 2.0 |
| | yl)isoquinolin-1-amine | | Hz, 1H), 7.30 (d, J = 2.4 Hz, 1H), 7.24 |
| | | | (dd, J = 9.2, 2.4 Hz, 1H), 7.16 (d, J = |
| | | | 6.0 Hz, 1H), 6.40 (d, J = 2.0 Hz, 1H), |
| | | | 5.18 (s, 2H), 3.91 (s, 3H), 2.65 (s, |
| | | | 3H), NH not observed. |

### Example 207

### Synthesis of 1-(((1-((2-chloropyrimidin-5-yl)amino)isoquinolin-6-yl)oxy)methyl)cyclopropane-1-carbonitrile

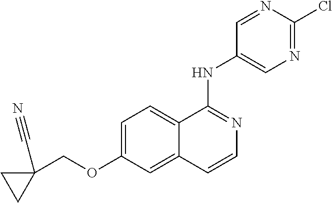 [SMILES: N#CC1(COc2ccc3c(Nc4cnc(Cl)nc4)nccc3c2)CC1]

### Step 1. Preparation of (1-cyanocyclopropyl)methyl 4-methylbenzenesulfonate

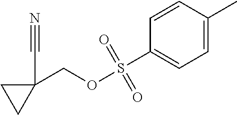 [SMILES: Cc1ccc(S(=O)(=O)OCC2(C#N)CC2)cc1]

To a solution of 1-(hydroxymethyl)cyclopropanecarbonitrile (1.04 g, 10.3 mmol) in dichloromethane (40 mL) was added p-toluenesulfonyl chloride (2.16 g, 11.3 mmol), 4-dimethylaminopyridine (0.126 g, 1.03 mmol), and triethylamine (1.65 mL, 11.8 mmol), and the reaction mixture was stirred at ambient temperature for 16 h. The reaction mixture was diluted with dichloromethane (40 mL), washed with water (50 mL), and saturated sodium chloride (50 mL). The organic phase was dried over anhydrous sodium sulfate, filtered, and the filtrate was concentrated in vacuo. The residue was purified by silica gel column chromatography, eluting with a gradient of 15 to 80% of ethyl acetate in heptane, to provide the title compound as a colorless oil (1.91 g, 74% yield): NMR (400 MHz, DMSO-d₆) δ7.81 (d, J=8.3 Hz, 2H), 7.50 (d, J=8.1 Hz, 2H), 4.12 (s, 2H), 2.43 (s, 3H), 1.32 (q, J=3.8 Hz, 2H), 1.08 (q, J=3.8 Hz, 2H).

### Step 2. Preparation of 1-(((1-chloroisoquinolin-6-yl)oxy)methyl)cyclopropane-1-carbonitrile

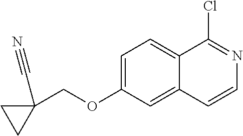 [SMILES: N#CC1(COc2ccc3c(Cl)nccc3c2)CC1]

To a solution of (1-cyanocyclopropyl)methyl 4-methylbenzenesulfonate (0.500 g, 1.99 mmol) in N,N-dimethylformamide (6.5 mL) was added potassium carbonate (0.550 g, 3.98 mmol) and 1-chloroisoquinolin-6-ol (0.393 mg, 2.19 mmol). The reaction mixture was heated to 80° C. for 16 h. After cooling to ambient temperature, the reaction mixture was diluted with ethyl acetate (20 mL), washed with water (20 mL), and saturated sodium chloride (20 mL). The organic phase was dried over anhydrous sodium sulfate, filtered, and the filtrate concentrated in vacuo. The residue was purified by silica gel column chromatography, eluting with a gradient of 0 to 40% of ethyl acetate in heptane, to provide the title compound as a colorless solid (0.491 g, 95% yield): NMR (400 MHz, DMSO-d₆) δ 8.23 (d, J=5.7 Hz, 1H), 8.20 (d, J=9.2 Hz, 1H), 7.75 (d, J=5.8 Hz, 1H), 7.52-7.49 (m, 1H), 7.47 (d, J=2.4 Hz, 1H), 4.25 (s, 2H), 1.43 (q, J=3.7 Hz, 2H), 1.23 (q, J=3.7 Hz, 2H). MS (ES+) m/z 258.8 (M+1), 260.8 (M+1).

### Step 3. Preparation of 1-(((1-((2-chloropyrimidin-5-yl)amino)isoquinolin-6-yl)oxy)methyl)cyclopropane-1-carbonitrile

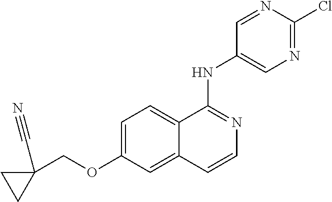 [SMILES: N#CC1(COc2ccc3c(Nc4cnc(Cl)nc4)nccc3c2)CC1]

To a solution of 1-(((1-chloroisoquinolin-6-yl)oxy)methyl)cyclopropane-1-carbonitrile (0.491 g, 1.90 mmol) in 1,4-dioxane (18 mL) was added 5-amino-2-chloropyrimidine (0.245 g, 1.90 mmol), tris(dibenzylideneacetone)dipalladium(0) (0.174 g, 0.189 mmol), 2-dicyclohexylphosphino-2′,6′-dimethoxy-1,1′-biphenyl (0.156 g, 0.380 mmol), and potassium phosphate tribasic (0.805 g, 3.80 mmol). The mixture was degassed by passing a stream of nitrogen through it for 5 minutes. The reaction mixture was heated to 110° C. for 1 h. After cooling to ambient temperature, the reaction mixture was filtered through a pad of celite, and the filtrate was concentrated in vacuo. The residue was purified by silica gel column chromatography, eluting with a gradient of 5 to 25% of methanol in dichloromethane. The obtained residue was then purified by reverse-phase preparative HPLC (Phenomenex Gemini-NX C18 150 mm×30 mm, 5 μm column), eluting with a gradient of 10 to 50% of acetonitrile in water containing 0.5% of formic acid, to provide the title compound as a colorless solid (0.085 g, 12% yield): ¹H NMR (400 MHz, DMSO-d₆) δ9.62 (s, 1H), 9.30 (s, 2H), 8.46 (d, J=9.2 Hz, 1H), 8.01 (d, J=5.8 Hz, 1H), 7.40 (dd, J=9.2, 2.5 Hz, 1H), 7.29 (d, J=2.5 Hz, 1H), 7.22 (d, J=5.8 Hz, 1H), 4.22 (s, 2H), 1.43 (q, J=3.7 Hz, 2H), 1.24-1.21 (m, 2H); MS (ES+) m/z 352.0 (M+1), 354.0 (M+1).

### Example 208

### Synthesis of 3-(((1-((2-chloropyrimidin-5-yl)amino)isoquinolin-6-yl)oxy)methyl)oxetane-3-carbonitrile

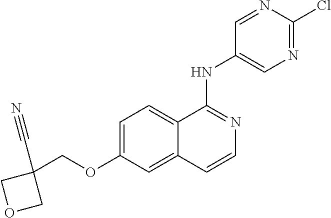 [SMILES: N#CC1(COc2ccc3c(Nc4cnc(Cl)nc4)nccc3c2)COC1]

### Step 1. Preparation of 3-(((1-chloroisoquinolin-6-yl)oxy)methyl)oxetane-3-carbonitrile

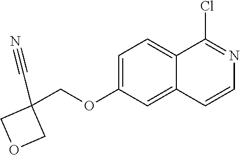 [SMILES: N#CC1(COc2ccc3c(Cl)nccc3c2)COC1]

To a solution of 3-(hydroxymethyl)oxetane-3-carbonitrile (0.260 g, 2.30 mmol) in N,N-dimethylformamide (9.5 mL) was added sodium hydride (60% dispersion in mineral oil, 0.092 g, 2.30 mmol) at 0° C. and the resulting mixture was stirred at this temperature for 30 minutes. To it was then added 1-chloro-6-fluoroisoquinoline (0.380 g, 2.09 mmol). The reaction mixture was allowed to warm to ambient temperature and stirred for 16h. The reaction mixture was diluted with ethyl acetate (50 mL), washed with saturated sodium bicarbonate solution (30 mL), and saturated sodium chloride (30 mL). The organic phase was dried over anhydrous sodium sulfate, filtered, and the filtrate was concentrated in vacuo. The obtained residue was purified by silica gel column chromatography, eluting with a gradient of 0 to 80% of ethyl acetate in heptane, to provide the title compound as a colorless solid (0.282 g, 49% yield): MS (ES+) m/z 275.6 (M+1), 277.6 (M+1).

### Step 2. Preparation of 3-(((1-((2-chloropyrimidin-5-yl)amino)isoquinolin-6-yl)oxy)methyl)oxetane-3-carbonitrile

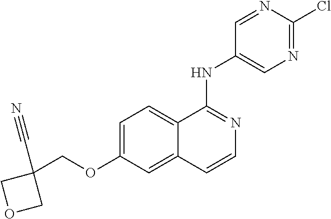 [SMILES: N#CC1(COc2ccc3c(Nc4cnc(Cl)nc4)nccc3c2)COC1]

To a solution of 3-(((1-chloroisoquinolin-6-yl)oxy)methyl)oxetane-3-carbonitrile (0.279 g, 1.016 mmol) in 1,4-dioxane (9 mL) was added 5-amino-2-chloropyrimidine (0.125 g, 0.965 mmol), tris(dibenzylideneacetone)dipalladium(0) (0.093 g, 0.102 mmol), 2-dicyclohexylphosphino-2′,6′-dimethoxy-1,1′-biphenyl (0.083 g, 0.203 mmol), and potassium phosphate tribasic (0.410 g, 1.93 mmol). The mixture was degassed by passing a stream of nitrogen through it for 5 minutes. The reaction mixture was heated to 110° C. for 1 h. After cooling to ambient temperature, the reaction mixture was filtered through a pad of celite, and the filtrate was concentrated in vacuo. The residue was purified by silica gel column chromatography, eluting with a gradient of 20 to 100% of ethyl acetate in heptane. The obtained residue was then purified by reverse-phase preparative HPLC (Phenomenex Gemini-NX C18 150 mm×30 mm, 5 μm column), eluting with a gradient of 15 to 80% of acetonitrile in water (containing 0.5% of formic acid), to provide the title compound as a colorless solid (0.065 g, 18% yield): ¹H NMR (400 MHz, DMSO-d₆) δ 9.63 (s, 1H), 9.30 (s, 2H), 8.48 (d, J=9.1 Hz, 1H), 8.03 (d, J=5.8 Hz, 1H), 7.43-7.38 (m, 2H), 7.26 (d, J=5.8 Hz, 1H), 4.94 (d, J=6.7 Hz, 2H), 4.72-4.70 (m, 4H); MS (ES+) m/z 368.0 (M+1), 370.0 (M+1).

### Example 209

### Synthesis of N-(6-chloropyridin-3-yl)-6-((4,4-dimethyloxetan-2-yl)methoxy)isoquinolin-1-amine

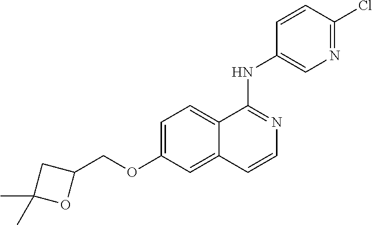 [SMILES: CC1(C)CC(COc2ccc3c(Nc4ccc(Cl)nc4)nccc3c2)O1]

### Step 1. Preparation of 1-chloro-6-((4,4-dimethyloxetan-2-yl)methoxy)isoquinoline

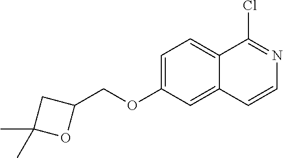 [SMILES: CC1(C)CC(COc2ccc3c(Cl)nccc3c2)O1]

To a solution of (4,4-dimethyloxetan-2-yl)methanol (0.100 g, 0.860 mmol) and triethylamine (0.262 g, 2.59 mmol) in dichloromethane (5 mL) was added methanesulfonyl chloride (0.200 g, 1.75 mmol) dropwise at 0° C. The reaction mixture was allowed to warm to ambient temperature and stirred for 1 h. The mixture was then poured into saturated sodium bicarbonate solution (20 mL) and extracted with ethyl acetate (3×20 mL). The combined organic layers were washed with brine (20 mL), dried over anhydrous sodium sulfate, and filtered. The filtrate was concentrated in vacuo to afford a colorless oil (0.160 g). To the oil was added N,N-dimethylformamide (2 mL), followed by 1-chloroisoquinolin-6-ol (0.149 g, 0.829 mmol) and potassium carbonate (0.342 g, 2.47 mmol). The reaction mixture was then heated to 90° C. for 2 h. After cooling to ambient temperature, the reaction mixture was poured into water (20 mL) and extracted with ethyl acetate (3×20 mL). The combined organic layers were washed with brine (10 mL), dried over anhydrous sodium sulfate, and filtered. Concentration of the filtrate and purification of the obtained residue by silica gel column chromatography, eluting with a gradient of 20 to 30% of ethyl acetate in petroleum ether, afforded the title compound as a colorless solid (0.260 g, quantitative yield): MS (ES+) m/z 278.1 (M+1), 280.1 (M+1).

### Step 2. Preparation of N-(6-chloropyridin-3-yl)-6-((4,4-dimethyloxetan-2-yl)methoxy)isoquinolin-1-amine

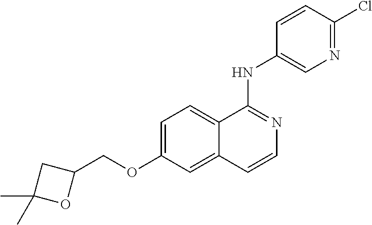 [SMILES: CC1(C)CC(COc2ccc3c(Nc4ccc(Cl)nc4)nccc3c2)O1]

To a mixture of 1-chloro-6-((4,4-dimethyloxetan-2-yl)methoxy)isoquinoline (0.220 g, 0.792 mmol) and 6-chloropyridin-3-amine (0.110 g, 0.855 mmol) in 2-methyl-2-butanol (3 mL) was added cesium carbonate (0.770 g, 2.36 mmol) and methanesulfonato(2-di-t-butylphosphino-2′,4′,6′-tri-i-propyl-1,1′-biphenyl)(2′-amino-1,1′-biphenyl-2-yl)palladium(II) (0.066 g, 0.083 mmol). The reaction mixture was heated to 90° C. for 12 h. After cooling to ambient temperature, the mixture was poured into water (20 mL) and extracted with ethyl acetate (3×20 mL). The combined organic layers were washed with brine (10 mL), dried over anhydrous sodium sulfate, and filtered. The filtrate was concentrated in vacuo. The obtained residue was purified by silica gel column chromatography, eluting with a gradient of 10 to 38% of ethyl acetate in petroleum ether, and then by reverse-phase preparative HPLC (Phenomenex Gemini-NX C18 75 mm×30 mm, 3 μm column), eluting with a gradient of 38 to 68% of acetonitrile in water containing 0.05% of ammonium hydroxide, to afford the title compound as a colorless solid (0.126 g, 42% yield): ¹H NMR (400 MHz, DMSO-d₆) δ9.39 (s, 1H), 8.88 (d, J=2.8 Hz, 1H), 8.49-8.36 (m, 2H), 7.96 (d, J=5.6 Hz, 1H), 7.44 (d, J=8.8 Hz, 1H), 7.35-7.26 (m, 2H), 7.17 (d, J=5.6 Hz, 1H), 4.95-4.79 (m, 1H), 4.30-4.16 (m, 2H), 2.45 (dd, J=8.0, 10.8 Hz, 1H), 2.40-2.33 (m, 1H), 1.41 (d, J=14 Hz, 6H); MS (ES+) m/z 370.1 (M+1), 372.1 (M+1).

### Examples 210-225

In a similar manner as described in EXAMPLE 209, utilizing the appropriately substituted starting materials and intermediates, the following compounds were prepared:

| | | MS | |
|---|---|---|---|
| | | (ES+) | |
| Compound No. | Name | m/z | NMR |
| 210 | N-(6-chloropyridin-3- | 356.1 | ¹H NMR (400 MHz, DMSO-d₆) δ 9.39 |
| | yl)-6-((3-methyloxetan- | (M + 1). | (s, 1H), 8.90 (dd, J = 2.8, 0.5 Hz, 1H), |
| | 3- | 358.1 | 8.47-8.42 (m, 2H), 7.98 (d, J = 5.8 Hz, |
| | yl)methoxy)isoquinolin- | (M + 1). | 1H), 7.45 (d, J = 8.7 Hz, 1H), 7.36- |
| | 1-amine | | 7.32 (m, 2H), 7.20 (d, J = 5.7 Hz, 1H), |
| | | | 4.56 (d, J = 5.8 Hz, 2H), 4.36 (d, J = |
| | | | 5.8 Hz, 2H), 4.23 (s, 2H), 3.33 (s, 3H). |
| 211 | N-(6-chloropyridin-3- | 353.2 | ¹H NMR (400 MHz, CD₃OD) 88.74 (d, |
| | yl)-6-(oxazol-2- | (M + 1), | J = 2.4 Hz, 1H), 8.31 (d, J = 9.3 Hz, |
| | ylmethoxy)isoquinolin- | 355.2 | 1H), 8.28 (dd, J = 8.7, 2.8 Hz, 1H), |
| | 1-amine | (M + 1). | 8.02 (d, J = 0.9 Hz, 1H), 7.94 (d, J = |
| | | | 5.9 Hz, 1H), 7.41 (dd, J = 8.7, 0.5 Hz, |
| | | | 1H), 7.37 (d, J = 2.6 Hz, 1H), 7.32 |
| | | | (dd, J = 9.2, 2.6 Hz, 1H), 7.27 (d, J = |
| | | | 0.8 Hz, 1H), 7.20 (d, J = 5.8 Hz, 1H), |
| | | | 5.38 (s, 2H), NH not observed. |
| 212 | N-(6-chloropyridin-3- | 369.0 | ¹H NMR (400 MHz, CD₃OD) δ 8.63- |
| | yl)-6-(thiazol-2- | (M + 1), 371 | 8.59 (m, 2H), 8.03 (dd, J = 8.5, 2.8 |
| | ylmethoxy)isoquinolin- | (M + 1). | Hz, 1H), 7.97 (d, J = 3.4 Hz, 1H), 7.81 |
| | 1-amine | | (d, J = 3.3 Hz, 1H), 7.73 (dd, J = 8.5, |
| | | | 0.6 Hz, 1H), 7.68-7.62 (m, 2H), 7.54 |
| | | | (d, J = 7.0 Hz, 1H), 7.41 (d, J = 6.6 |
| | | | Hz, 1H), 5.75 (s, 2H), NH not |
| | | | observed. |
| 213 | N-(2-chloropyrimidin-5- | 343.2 | ¹H NMR (400 MHz, DMSO-d₆) δ 9.62 |
| | yl)-6-(oxetan-3- | (M + 1), | (s, 1H), 9.31 (s, 2H), 8.45 (d, J = 9.1 |
| | ylmethoxy)isoquinolin- | 345.2 | Hz, 1H), 8.01 (d, J = 5.8 Hz, 1H), 7.37 |
| | 1-amine | (M + 1). | (d, J = 2.4 Hz, 1H), 7.34 (dd, J = 9.1, |
| | | | 2.6 Hz, 1H), 7.25 (d, J = 5.8 Hz, 1H), |
| | | | 4.76 (dd, J = 7.9, 6.1 Hz, 2H), 4.49 (t, |
| | | | J = 6.1 Hz, 2H), 4.39 (d, J = 6.7 Hz, |
| | | | 2H), 3.53-3.43 (m, 1H). |
| 214 | N-(6-methylpyridin-3- | 322.2 | ¹H NMR (400 MHz, DMSO-d₆) δ 9.14 |
| | yl)-6-(oxetan-3- | (M + 1). | (s, 1H), 8.84 (d, J = 2.4 Hz, 1H), 8.45 |
| | ylmethoxy)isoquinolin- | | (d, J = 9.2 Hz, 1H), 8.20 (dd, J = 8.4, |
| | 1-amine | | 2.7 Hz, 1H), 7.92 (d, J = 5.8 Hz, 1H), |
| | | | 7.30 (d, J = 2.6 Hz, 1H), 7.26 (dd, J = |
| | | | 9.2, 2.6 Hz, 1H), 7.19 (d, J = 8.4 Hz, |
| | | | 1H), 7.11 (d, J = 5.7 Hz, 1H), 4.76 |
| | | | (dd, J = 7.9, 6.1 Hz, 2H), 4.48 (t, J = |
| | | | 6.1 Hz, 2H), 4.37 (d, J = 6.7 Hz, 2H), |
| | | | 3.51-3.43 (m, 1H), 2.43 (s, 3H). |
| 215 | N-(6-chloropyridin-3- | 353.2 | ¹H NMR (400 MHz, DMSO-d₆) δ 9.40 |
| | yl)-6-(isoxazol-3- | (M + 1), | (s, 1H), 9.00 (d, J = 1.6 Hz, 1H), 8.88 |
| | ylmethoxy)isoquinolin- | 355.2 | (d, J = 2.8 Hz, 1H), 8.48 (d, J = 9.3 |
| | 1-amine | (M + 1). | Hz, 1H), 8.42 (dd, J = 8.7, 2.9 Hz, |
| | | | 1H), 7.99 (d, J = 5.7 Hz, 1H), 7.49- |
| | | | 7.42 (m, 2H), 7.38-7.36 (m, 1H), 7.19 |
| | | | (d, J = 5.6 Hz, 1H), 6.78 (d, J = 1.6 |
| | | | Hz, 1H), 5.41 (s, 2H). |
| 216 | N-(6-chloropyridin-3- | 356.1 | ¹H NMR (400 MHz, CD₃OD) 88.74 (d, |
| | yl)-6-((tetrahydro-2H- | (M + 1), | J = 2.8 Hz, 1H), 8.32-8.23 (m, 2H), |
| | pyran-3- | 358.1 | 7.91 (d, J = 5.9 Hz, 1H), 7.41 (d, J = |
| | yl)oxy)isoquinolin-1- | (M + 1). | 8.7 Hz, 1H), 7.31-7.23 (m, 2H), 7.17 |
| | amine | | (d, J = 6.0 Hz, 1H), 4.69-4.58 (m, 1H), |
| | | | 4.04-3.93 (m, 1H), 3.83-3.64 (m, 3H), |
| | | | 2.25-2.10 (m, 1H), 2.04-1.87 (m, 2H), |
| | | | 1.77-1.62 (m, 1H), NH not observed. |
| 217 | N-(6-chloropyridin-3- | 356.2 | ¹H NMR (400 MHz, DMSO-d₆) δ 11.5 |
| | yl)-6-((tetrahydrofuran- | (M + 1), | (s, 1H), 8.84 (d, J = 9.3 Hz, 1H), 8.68 |
| | 2- | 358.2 | (d, J = 2.7 Hz, 1H), 8.11 (dd, J = 8.5, |
| | yl)methoxy)isoquinolin- | (M + 1). | 2.7 Hz, 1H), 7.73 (d, J = 8.6 Hz, 1H), |
| | 1-amine | | 7.62 (d, J = 6.9 Hz, 1H), 7.55 (d, J = |
| | | | 2.4 Hz, 1H), 7.49 (dd, J = 9.2, 2.5 Hz, |
| | | | 1H), 7.32 (d, J = 6.9 Hz, 1H), 4.30- |
| | | | 4.20 (m, 2H), 4.20-4.13 (m, 1H), 3.86- |
| | | | 3.77 (m, 1H), 3.77-3.67 (m, 1H), 2.11- |
| | | | 2.02 (m, 1H), 1.99-1.82 (m, 2H), 1.76- |
| | | | 1.67 (m, 1H). |
| 218 | 6-((3-methyloxetan-3- | 337.2 | ¹H NMR (400 MHz, DMSO-d₆) δ 9.30 |
| | yl)methoxy)-N-(2- | (M + 1). | (s, 1H), 9.16 (s, 2H), 8.44 (d, J = 8.8 |
| | methylpyrimidin-5- | | Hz, 1H), 7.95 (d, J = 5.8 Hz, 1H), |
| | yl)isoquinolin-1-amine | | 7.35-7.30 (m, 2H), 7.17 (d, J = 5.8 |
| | | | Hz, 1H), 4.55 (d, J = 5.8 Hz, 2H), 4.35 |
| | | | (d, J = 5.8 Hz, 2H), 4.22 (s, 2H), 2.57 |
| | | | (s, 3H), 1.41 (s, 3H). |
| 219 | 6-((3-methoxyoxetan-3- | 353.3 | ¹H NMR (400 MHz, DMSO-d₆) δ 9.31 |
| | yl)methoxy)-N-(2- | (M + 1) | (s, 1H), 9.16 (s, 2H), 8.45 (d, J = 9.2 |
| | methylpyrimidin-5- | | Hz, 1H), 8.19 (s, 0.3H), 7.96 (d, J = |
| | yl)isoquinolin-1-amine | | 5.8 Hz, 1H), 7.38 (d, J = 2.4 Hz, 1H), |
| | formate | | 7.33 (dd, J=9.2, 2.4, Hz, 1H), 7.16 (d, |
| | | | J = 5.6 Hz, 1H), 4.65 (d, J = 7.2 Hz, |
| | | | 2H), 4.53 (d, J = 7.2 Hz, 2H), 4.47 (s, |
| | | | 2H), 3.32 (s, 3H), 2.57 (s, 3H). |
| 220 | N-(6-chloropyridin-3- | 372.2 | ¹H NMR (400 MHz, DMSO-d₆) δ 9.40 |
| | yl)-6-((3- | (M + 1), | (s, 1H), 8.89 (d, J = 2.4 Hz, 1H), |
| | methoxyoxetan-3- | 374.1 | 8.49-8.41 (m, 2H), 7.98 (d, J = 5.6 |
| | yl)methoxy)isoquinolin- | (M + 1). | Hz, 1H), 7.44 (d, J = 8.4 Hz, 1H), 7.39 |
| | 1-amine | | (d, J = 2.4 Hz, 1H), 7.33 (dd, J = 9.2, |
| | | | 2.4 Hz, 1H), 7.18 (d, J = 5.6 Hz, 1H), |
| | | | 4.65 (d, J = 7.2 Hz, 2H), 4.53 (d, J = |
| | | | 7.2 Hz, 2H), 4.47 (s, 2H), 3.32 (s, 3H). |
| 221 | N-(2-methylpyrimidin-5- | 323.2 | ¹H NMR (400 MHz, DMSO-d₆) δ 9.31 |
| | yl)-6-(oxetan-3- | (M + 1). | (s, 1H), 9.16 (s, 2H), 8.43 (d, J = 9.2 |
| | ylmethoxy)isoquinolin- | | Hz, 1H), 7.95 (d, J = 5.6 Hz, 1H), |
| | 1-amine | | 7.33-7.28 (m, 2H), 7.16 (d, J = 5.6 |
| | | | Hz, 1H), 4.77-4.73 (m, 2H), 4.48 (t, J = |
| | | | 6.0 Hz, 2H), 4.37 (d, J = 6.8 Hz, |
| | | | 2H), 3.50-3.43 (m, 1H), 2.57 (s, 3H). |
| 222 | 6-(isoxazol-3- | 334.3 | ¹H NMR (400 MHz, DMSO-d₆) δ 9.32 |
| | ylmethoxy)-N-(2- | (M + 1). | (s, 1H), 9.15 (s, 2H), 8.99 (d, J = 1.6 |
| | methylpyrimidin-5- | | Hz, 1H), 8.45 (d, J = 9.2 Hz, 1H), 7.96 |
| | yl)isoquinolin-1-amine | | (d, J = 5.6 Hz, 1H), 7.43 (d, J = 2.4 |
| | | | Hz, 1H), 7.36 (dd, J = 9.2, 2.4 Hz, |
| | | | 1H), 7.17 (d, J = 6.0 Hz, 1H), 6.77 (d, |
| | | | J = 1.6 Hz, 1H), 5.41 (s, 2H), 2.58 (s, 3H). |
| 223 | N-(6-chloropyridin-3- | 342.2 | ¹H NMR (400 MHz, DMSO-d₆) δ 9.39 |
| | yl)-6-(oxetan-3- | (M + 1), | (s, 1H), 8.89 (d, J = 2.8 Hz, 1H), 8.46- |
| | ylmethoxy)isoquinolin- | 344.2 | 8.42 (m, 2H), 7.98 (d, J = 5.8 Hz, 1H), |
| | 1-amine | (M + 1). | 7.45 (d, J = 8.7 Hz, 1H), 7.34 (d, J = |
| | | | 2.4 Hz, 1H), 7.30 (dd, J = 9.1, 2.5 Hz, |
| | | | 1H), 7.19 (d, J = 5.8 Hz, 1H), 4.76 |
| | | | (dd, J = 7.9, 6.1 Hz, 2H), 4.49 (t, J = |
| | | | 6.0 Hz, 2H), 4.38 (d, J = 6.7 Hz, 2H), |
| | | | 3.51-3.44 (m, 1H). |
| 224 | (R)-3-((1-((6- | 398.1 | ¹H NMR (400 MHz, DMSO-d₆) δ 9.41 |
| | chloropyridin-3- | (M + 1), | (s, 1H), 8.89-8.89 (m, 1H), 8.48-8.42 |
| | yl)amino)isoquinolin-6- | 400.1 | (m, 2H), 7.98 (d, J = 5.7 Hz, 1H), 7.45 |
| | yl)oxy)-1,1,1-trifluoro-2- | (M + 1). | (d, J = 8.8 Hz, 1H), 7.38-7.38 (m, 1H), |
| | methylpropan-2-ol | | 7.34-7.31 (m, 1H), 7.21 (d, J = 5.7 Hz, |
| | | | 1H), 6.43 (s, 1H), 4.24-4.16 (m, 2H), |
| | | | 1.48 (s, 3H); ¹⁹F NMR ( 376 MHz, |
| | | | DMSO-d₆) δ −79.5 (s). |
| 225 | (S)-3-((1-((6- | 398.1 | ¹H NMR (400 MHz, DMSO-d₆) δ 9.40 |
| | chloropyridin-3- | (M + 1), | (s, 1H), 8.89 (d, J = 2.8 Hz, 1H), 8.48- |
| | yl)amino)isoquinolin-6- | 400.1 | 8.42 (m, 2H), 7.98 (d, J = 5.7 Hz, 1H), |
| | yl)oxy)-1,1,1-trifluoro-2- | (M + 1). | 7.45 (d, J = 8.7 Hz, 1H), 7.38 (d, J = |
| | methylpropan-2-ol | | 2.4 Hz, 1H), 7.32 (dd, J = 9.2, 2.4 Hz, |
| | | | 1H), 7.21 (d, J = 5.9 Hz, 1H), 6.43- |
| | | | 6.42 (m, 1H), 4.25-4.16 (m, 2H), 1.48 |
| | | | (s, 3H); ¹⁹F NMR ( 376 MHz, DMSO- |
| | | | d₆) δ −79.5 (s). |

### Example 226

### Synthesis of 6-(2-amino-3,3,3-trifluoropropoxy)-N-(6-chloropyridin-3-yl)isoquinolin-1-amine

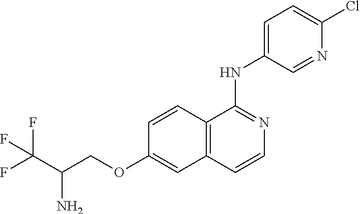 [SMILES: NC(COc1ccc2c(Nc3ccc(Cl)nc3)nccc2c1)C(F)(F)F]

### Step 1. Preparation of tert-butyl (1,1,1-trifluoro-3-hydroxypropan-2-yl)carbamate

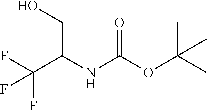

A mixture of 2-amino-3,3,3-trifluoro-propan-1-ol hydrochloride (0.500 g, 3.02 mmol), triethylamine (0.916 g, 9.05 mmol), and di-tert-butyl dicarbonate (0.660 g, 3.02 mmol) in dichloromethane (10.0 mL) was stirred at ambient temperature for 12 h. The mixture diluted with water (20 mL) and extracted with ethyl acetate (3×50 mL). The combined organic phase was washed with saturated citric acid solution (50 mL), saturated sodium bicarbonate solution (50 mL), and brine (100 mL). The organic phase was dried over anhydrous sodium sulfate and filtered. The filtrate was concentrated in vacuo to afford the title compound as yellowish oil (0.500 g), which was used for the next step without further purification: ¹H NMR (400 MHz, CDCl₃) δ5.22-5.02 (m, 1H), 4.68-4.50 (m, 1H), 4.40-4.24 (m, 1H), 4.05-3.79 (m, 2H), 1.49-1.47 (m, 9H).

### Step 2. Preparation of 2-((tert-butoxycarbonyl)amino)-3,3,3-trifluoropropyl 4-methylbenzenesulfonate

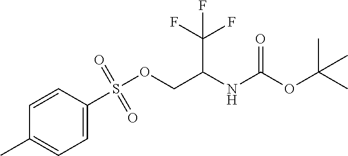 [SMILES: Cc1ccc(S(=O)(=O)OCC(NC(=O)OC(C)(C)C)C(F)(F)F)cc1]

To a solution of tert-butyl (1,1,1-trifluoro-3-hydroxypropan-2-yl)carbamate (0.800 g, 3.49 mmol) and 4-methylbenzenesulfonyl chloride (0.670 g, 3.51 mmol) in dichloromethane (15.0 mL) was added N,N-dimethylpyridin-4-amine (0.0420 g, 0.344 mmol) and triethylamine (1.06 g, 10.5 mmol) at 0° C. The mixture was allowed to warm to ambient temperature and stirred for 12 h. The mixture was concentrated in vacuo to afford a residue which was purified by silica gel column chromatography, eluting with a gradient of 0 to 10% of ethyl acetate in petroleum ether, to provide the title compound as a colorless solid (0.480 g, 36% yield): ¹H NMR (400 MHz, CDCl₃) δ7.80 (d, J=8.4 Hz, 2H), 7.38 (d, J=8.4 Hz, 2H), 5.04 (d, J=9.6 Hz, 1H), 4.62-4.37 (m, 1H), 4.28-4.16 (m, 2H), 2.47 (s, 3H), 1.46 (s, 9H).

### Step 3. Preparation of tert-butyl (3-((1-((6-chloropyridin-3-yl)amino)isoquinolin-6-yl)oxy)-1,1,1-trifluoropropan-2-yl)carbamate

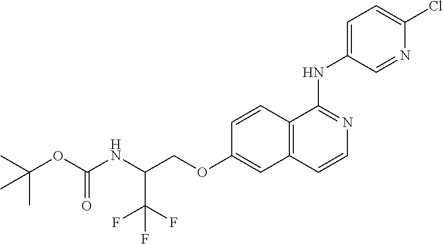 [SMILES: CC(C)(C)OC(=O)NC(COc1ccc2c(Nc3ccc(Cl)nc3)nccc2c1)C(F)(F)F]

A mixture of 2-((tert-butoxycarbonyl)amino)-3,3,3-trifluoropropyl 4-methylbenzenesulfonate (0.480 g, 1.25 mmol), 1-((6-chloropyridin-3-yl)amino)isoquinolin-6-ol (0.380 g, 1.40 mmol), and potassium carbonate (0.345 g, 2.50 mmol) in N,N-dimethylformamide (5 mL) was heated to 80° C. for 12 h. After cooling to ambient temperature, the mixture was diluted with water (20 mL) and extracted with ethyl acetate (3×30 mL). The combined organic layers were washed with brine (3×100 mL), dried over anhydrous sodium sulfate, and filtered. The filtrate was concentrated in vacuo to afford a residue which was purified by silica gel column chromatography, eluting with a gradient of 0 to 45% of ethyl acetate in petroleum ether, to provide the title compound as a colorless solid (0.100 g, 17% yield): ¹H NMR (400 MHz, CDCl₃) δ 8.54 (d, J=2.8 Hz, 1H), 8.39 (dd, J=2.8, 8.8 Hz, 1H), 8.08 (d, J=5.6 Hz, 1H), 7.91 (d, J=9.2 Hz, 1H), 7.34 (d, J=8.8 Hz, 1H), 7.25 (dd, J=2.4, 9.2 Hz, 1H), 7.15 (d, J=6.0 Hz, 1H), 7.09 (d, J=2.0 Hz, 1H), 5.32-5.23 (m, 1H), 4.84-4.69 (m, 1H), 4.43-4.27 (m, 2H), 1.49 (s, 9H), NH not observed.

### Step 4. 6-(2-amino-3,3,3-trifluoropropoxy)-N-(6-chloropyridin-3-yl)isoquinolin-1-amine

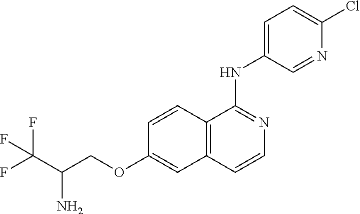 [SMILES: NC(COc1ccc2c(Nc3ccc(Cl)nc3)nccc2c1)C(F)(F)F]

To tert-butyl (3-((1-((6-chloropyridin-3-yl)amino)isoquinolin-6-yl)oxy)-1,1,1-trifluoropropan-2-yl)carbamate (0.0500 g, 0.103 mmol) was added a 4 M solution of hydrogen chloride in 1,4-dioxane (2.8 mL, 11.2 mmol) and the mixture was stirred at ambient temperature for 12 h. The mixture was concentrated in vacuo to afford a residue which was purified by reverse-phase preparative HPLC (Waters XBridge 150 mm×25 mm, 10 μm column), eluting with a gradient of 33 to 63% of acetonitrile in water (containing 10 mM of ammonium bicarbonate), to provide the title compound as an off-white solid (0.0169 g, 42% yield): ¹H NMR (400 MHz, CD₃OD) δ 8.73 (d, J=2.8 Hz, 1H), 8.41-8.20 (m, 2H), 7.92 (d, J=5.6 Hz, 1H), 7.40 (d, J=8.8 Hz, 1H), 7.32-7.27 (m, 1H), 7.27-7.25 (m, 1H), 7.20 (d, J=5.6 Hz, 1H), 4.42-4.22 (m, 2H), 3.86-3.70 (m, 1H), NH and NH₂ not observed; ¹⁹F NMR (376 MHz, CD₃OD) δ−77.0 (s); MS (ES+) m/z 383.1 (M+1), 385.1 (M+1).

### Example 227

### Synthesis of (R)—N-(6-chloropyridin-3-yl)-6-((4,4-difluoropyrrolidin-2-yl)methoxy)isoquinolin-1-amine trifluoroacetate

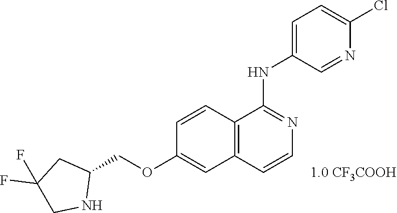 [SMILES: FC1(F)CN[C@@H](COc2ccc3c(Nc4ccc(Cl)nc4)nccc3c2)C1]

To a solution of (R)-(4,4-difluoropyrrolidin-2-yl)methanol hydrochloride (0.179 g, 1.03 mmol) in N,N-dimethylformamide (4.2 mL) was added potassium tert-butoxide (0.231 g, 2.06 mmol) at ambient temperature and the resulting mixture was stirred for 15 minutes at this temperature. To the reaction mixture was then added N-(6-chloropyridin-3-yl)-6-fluoro-N-((2-(trimethylsilyl)ethoxy)methyl)isoquinolin-1-amine (0.170 g, 0.421 mmol) and the reaction mixture was stirred at ambient temperature for 4h. The reaction mixture was quenched by addition of water (10 mL) and diluted with ethyl acetate (25 mL). The organic phase was washed with saturated sodium bicarbonate solution (15 mL), and brine (15 mL), dried over anhydrous sodium sulfate and filtered. The filtrate was concentrated in vacuo to give a colorless oil. To the residue was added dichloromethane (2 mL) and trifluoroacetic acid (0.65 mL) and the reaction mixture was stirred at ambient temperature for 2 h. Concentration of the mixture under reduced pressure and purification of the obtained residue by silica gel column chromatography, eluting with a gradient of 10 to 100% of ethyl acetate in heptane, provided the title compound as a colorless solid (0.207 g, 97% yield): ¹H NMR (400 MHz, DMSO-d₆) δ10.11 (m, 2H), 8.81 (d, J=2.7 Hz, 1H), 8.55 (d, J=9.2 Hz, 1H), 8.32 (dd, J=8.7, 2.6 Hz, 1H), 7.88 (d, J=6.0 Hz, 1H), 7.55 (d, J=8.7 Hz, 1H), 7.43 (d, J=2.3 Hz, 1H), 7.43-7.37 (m, 1H), 7.26 (d, J=6.2 Hz, 1H), 4.55-4.50 (m, 1H), 4.45-4.37 (m, 2H), 3.96-3.78 (m, 2H), 2.91-2.79 (m, 1H), 2.62-2.51 (m, 1H); ¹⁹F NMR (376 MHz, DMSO-d₆) δ−74.1 (s, 3F), −94.6 (d, J=234.5 Hz, 1F), −97.0 (d, J=234.5 Hz, 1F); MS (ES+) m/z 391.0 (M+1), 393.0 (M+1).

### Example 228

### Synthesis of 3-(((1-((2-methylpyrimidin-5-yl)amino)isoquinolin-6-yl)oxy)methyl)oxetane-3-carbonitrile

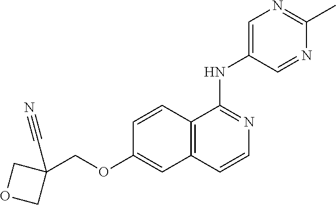 [SMILES: Cc1ncc(Nc2nccc3cc(OCC4(C#N)COC4)ccc23)cn1]

### Step 1. Preparation of 6-fluoro-N-(2-methylpyrimidin-5-yl)-N-((2-(trimethylsilyl)ethoxy)methyl)isoquinolin-1-amine

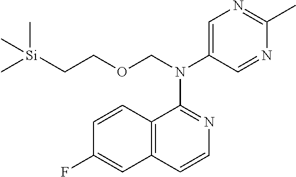

Following the procedure as described for EXAMPLE 76, Step 1 and 2, and making variations as required to replace 5-amino-2-chloropyridine with 2-methylpyrimidin-5-amine, the title compound was obtained as a colorless solid (30.0 g, 51% yield): ¹H NMR (400 MHz, DMSO-d₆) δ 8.35 (d, J=5.6 Hz, 1H), 8.25 (s, 2H), 7.92-7.83 (m, 2H), 7.74 (d, J=5.6 Hz, 1H), 7.48 (dt, J=8.0, 2.4 Hz, 1H), 5.37 (s, 2H), 3.54 (t, J=8.0 Hz, 2H), 2.52 (s, 3H), 0.78 (t, J=8.0 Hz, 2H), −0.15 (s, 9H); MS (ES+) m/z 385.2 (M+1).

### Step 2. Preparation of 3-(((1-((2-methylpyrimidin-5-yl)amino)isoquinolin-6-yl)oxy)methyl)oxetane-3-carbonitrile

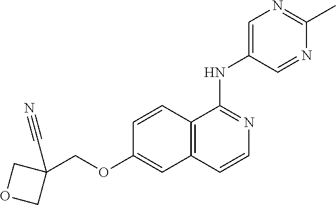 [SMILES: Cc1ncc(Nc2nccc3cc(OCC4(C#N)COC4)ccc23)cn1]

To a solution of 3-(hydroxymethyl)oxetane-3-carbonitrile (0.055 g, 0.488 mmol) in N,N-dimethylformamide (3 mL) was added sodium hydride (60% dispersion in mineral oil, 0.0260 g, 0.650 mmol) at 0° C. and the resulting mixture was stirred for 15 minutes at this temperature. To the reaction mixture was added 6-fluoro-N-(2-methylpyrimidin-5-yl)-N-((2-(trimethylsilyl)ethoxy)methyl)isoquinolin-1-amine (0.125 g, 0.325 mmol) and the reaction mixture was stirred at ambient temperature for 1h and then heated to 60° C. for 1 h. After cooling to ambient temperature, the reaction mixture was concentrated in vacuo to provide a colorless residue. To this residue was added dichloromethane (3 mL) and trifluoroacetic acid (1.25 mL) and the mixture was stirred at ambient temperature for 2 h. The reaction mixture was diluted with ethyl acetate (20 mL) and washed with saturated sodium bicarbonate solution (15 mL) and saturated sodium chloride (15 mL). The organic phase was dried over anhydrous sodium sulfate, filtered, and the filtrate was concentrated in vacuo. The residue was purified by silica gel column chromatography, eluting with a gradient of 10 to 100% of ethyl acetate in heptane, to provide the title compound as a colorless solid (0.0560 g, 50% yield): ¹H NMR (400 MHz, DMSO-d₆) δ 9.36 (s, 1H), 9.17 (s, 2H), 8.47 (d, J=9.2 Hz, 1H), 7.97 (d, J=5.8 Hz, 1H), 7.39-7.35 (m, 2H), 7.18 (d, J=5.8 Hz, 1H), 4.94 (d, J=6.6 Hz, 2H), 4.70 (m 4H), 2.58 (s, 3H); MS (ES+) m/z 348.2 (M+1).

### Example 229

### Synthesis of N-(6-chloropyridin-3-yl)-6-(cyclopropylmethoxy)-5-fluoroisoquinolin-1-amine

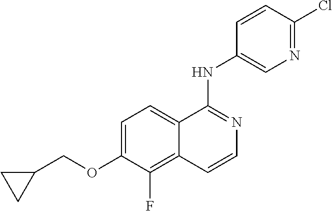 [SMILES: Fc1c(OCC2CC2)ccc2c(Nc3ccc(Cl)nc3)nccc12]

### Step 1. Preparation of 1-chloro-5-fluoroisoquinolin-6-ol)

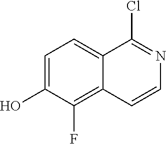 [SMILES: Oc1ccc2c(Cl)nccc2c1F]

To a solution of 1-chloro-5-fluoro-6-methoxy-isoquinoline (2.00 g, 9.45 mmol, prepared according to PCT Published Patent Application No. WO 2003/099274 A1) in dichloromethane (20 mL) was added tribromoborane (23.7 g, 94.5 mmol) dropwise at 15° C. The mixture was stirred at 15° C. for 12 h, poured into ice-water (100 mL), and extracted with ethyl acetate (5×50 mL). The combined organic layer was washed with brine (50 mL), dried over anhydrous sodium sulfate, filtered, and concentrated in vacuo to afford the title compound as a pale yellow solid (2.40 g, crude) that was used in the next step without further purification: ¹H NMR (400 MHz, DMSO-d₆) δ11.17 (s, 1H), 8.23 (d, J=6.0 Hz, 1H), 8.05-7.96 (m, 1H), 7.79 (d, J=6.0 Hz, 1H), 7.58-7.46 (m, 1H); MS (ES+) m/z 198.1 (M+1), 200.1 (M+1).

### Step 2. Preparation of 1-chloro-6-(cyclopropylmethoxy)-5-fluoroisoquinoline

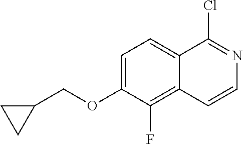 [SMILES: Fc1c(OCC2CC2)ccc2c(Cl)nccc12]

To a mixture of 1-chloro-5-fluoro-isoquinolin-6-ol (1.00 g, 5.06 mmol) and potassium carbonate (1.40 g, 10.1 mmol) in N,N-dimethylformamide (10 mL) was added bromomethylcyclopropane (0.820 g, 6.07 mmol) dropwise at 15° C. The mixture was stirred at 90° C. for 4 h, cooled to ambient temperature, and poured into water (20 mL). The mixture was extracted with ethyl acetate (3×20 mL). The combined organic layers were washed with brine (20 mL), dried over anhydrous sodium sulfate, filtered and concentrated in vacuo. The residue was purified by silica gel column chromatography, using 5% ethyl acetate in heptane as eluent, to afford the final compound as a colorless oil: ¹H NMR (400 MHz, CDCl₃) δ8.24 (d, J=6.0 Hz, 1H), 8.10 (d, J=9.2 Hz, 1H), 7.78 (d, J=5.6 Hz, 1H), 7.49-7.39 (m, 1H), 4.12 (d, J=7.2 Hz, 2H), 1.42-1.30 (m, 1H), 0.75-0.65 (m, 2H), 0.45-0.38 (m, 2H).

### Step 3. Preparation of N-(6-chloropyridin-3-yl)-6-(cyclopropylmethoxy)-5-fluoroisoquinolin-1-amine

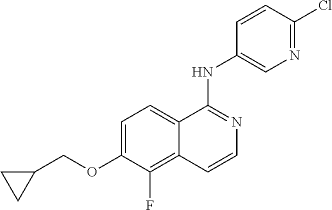 [SMILES: Fc1c(OCC2CC2)ccc2c(Nc3ccc(Cl)nc3)nccc12]

Following the procedure as described for EXAMPLE 1, Step 2 and making variations as required to replace 1-chloro-6-isopropoxyisoquinoline with 1-chloro-6-(cyclopropylmethoxy)-5-fluoroisoquinoline, the title compound was obtained as a colorless solid (0.257 g, 36% yield): ¹H NMR (400 MHz, DMSO-d₆) δ 9.49 (s, 1H), 8.87 (d, J=2.8 Hz, 1H), 8.44-8.38 (m, 1H), 8.34 (d, J=9.2 Hz, 1H), 8.01 (d, J=6.0 Hz, 1H), 7.61 (t, J=8.8 Hz, 1H), 7.46 (d, J=8.8 Hz, 1H), 7.23 (d, J=6.0 Hz, 1H), 4.12 (d, J=7.2 Hz, 2H), 1.36-1.24 (m, 1H), 0.65-0.58 (m, 2H), 0.42-0.35 (m, 2H); MS (ES+) m/z 344.2 (M+1), 346.2 (M+1).

### Example 230

### Synthesis of N¹-(6-chloropyridin-3-yl)-N⁶-(cyclopropylmethyl)isoquinoline-1,6-diamine formate

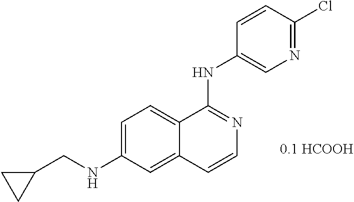 [SMILES: Clc1ccc(Nc2nccc3cc(NCC4CC4)ccc23)cn1]

### Step 1. Preparation of 6-((cyclopropylmethyl)amino)isoquinolin-1(2H)-one

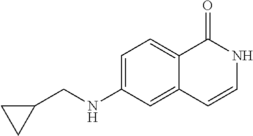 [SMILES: O=c1[nH]ccc2cc(NCC3CC3)ccc12]

To a mixture of 6-bromoisoquinolin-1(2H)-one (0.500 g, 2.23 mmol) and cyclopropylmethanamine (0.317 g, 4.46 mmol) in 1,4-dioxane (5 mL) was added [(2-di-tert-butylphosphino-2′,4′,6′-triisopropyl-1,1′-biphenyl)-2-(2′-amino-1,1′-biphenyl)] palladium(II) methanesulfonate (0.202 g, 0.223 mmol) and sodium tert-butoxide (0.643 g, 6.69 mmol) in glove box. The mixture was stirred at 90° C. for 12 h, cooled to ambient temperature, diluted with saturated ammonium chloride, and extracted with ethyl acetate (3×20 mL). The combined organic layers were washed with brine (20 mL), dried over anhydrous sodium sulfate, filtered, and concentrated in vacuo. The residue was purified by silica gel column chromatography, eluting with 17% ethyl acetate in petroleum ether, to afford the title compound as a yellow solid (0.340 g, 68% yield): ¹H NMR (400 MHz, DMSO-d₆) δ 10.63 (d, J=5.2 Hz, 1H), 7.82 (d, J=8.8 Hz, 1H), 6.98-6.92 (m, 1H), 6.76 (dd, J=2.2, 8.8 Hz, 1H), 6.53-6.46 (m, 2H), 6.25 (d, J=7.2 Hz, 1H), 2.97 (t, J=6.0 Hz, 2H), 1.13-1.02 (m, 1H), 0.53-0.44 (m, 2H), 0.23 (q, J=4.8 Hz, 2H).

### Step 2. Preparation of 1-chloro-N-(cyclopropylmethyl)-1,2-dihydroisoquinolin-6-amine

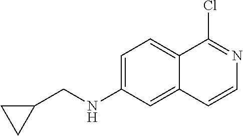 [SMILES: Clc1nccc2cc(NCC3CC3)ccc12]

A mixture of 6-((cyclopropylmethyl)amino)isoquinolin-1(2H)-one (0.100 g, 0.467 mmol) and phosphorus (V) oxychloride (1.65 g, 10.8 mmol) was stirred at 100° C. for 4 h. The reaction mixture was cooled to ambient temperature and concentrated in vacuo. The residue was cooled to 0° C., diluted with ice-water and dichloromethane, and a 10% aqueous sodium carbonate solution was added slowly with stirring until the aqueous layer was made alkaline. The mixture was extracted with ethyl acetate (3×10 mL). The combined organic layers were dried over anhydrous sodium sulfate, filtered, and concentrated in vacuo. The residue was purified by preparative thin layer chromatography, eluting with 25% ethyl acetate in petroleum ether, to afford the title compound as a dark brown solid (0.0600 g, 43% yield): ¹H NMR (400 MHz, CDCl₃) δ 8.10-8.01 (m, 2H), 7.31 (d, J=5.6 Hz, 1H), 6.98 (d, J=9.2 Hz, 1H), 6.64 (s, 1H), 4.43 (s, 1H), 3.09 (t, J=6.0 Hz, 2H), 1.22-1.09 (m, 1H), 0.69-0.53 (m, 2H), 0.32 (d, J=4.8 Hz, 2H); MS (ES+) m/z 233.2 (M+1), 235.2 (M+1).

### Step 3. Preparation of N¹-(6-chloropyridin-3-yl)-N⁶-(cyclopropylmethyl)isoquinoline-1,6-diamine formate

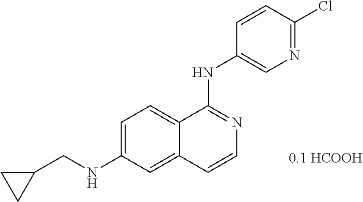 [SMILES: Clc1ccc(Nc2nccc3cc(NCC4CC4)ccc23)cn1]

To a mixture of 1-chloro-N-(cyclopropylmethyl)isoquinolin-6-amine (0.0400 g, 0.172 mmol) and 6-chloropyridin-3-amine (0.0221 g, 0.172 mmol) in 2-methylbutan-2-ol (0.4 mL)) was added [(2-di-tert-butylphosphino-2′,4′,6′-triisopropyl-1,1′-biphenyl)-2-(2′-amino-1,1′-biphenyl)]palladium(II) methanesulfonate (0.0137 g, 0.172 mmol) and cesium carbonate (0.280 g, 0.859 mmol) in glove box. The mixture was stirred at 70° C. for 12 h, cooled to ambient temperature, and concentrated in vacuo. The residue was purified by preparative reverse-phase HPLC, using acetonitrile in water containing 0.225% of formic acid as eluent, to afford the title compound as a grey solid (0.0075 g, 12% yield): ¹H NMR (400 MHz, DMSO-d₆) δ8.78 (br s, 1H), 8.31 (dd, J=1.6, 3.6 Hz, 1H), 8.21 (d, J=9.2 Hz, 1H), 8.14 (s, 0.1H), 7.72-7.63 (m, 1H), 7.49 (d, J=8.0 Hz, 1H), 7.09 (d, J=8.4 Hz, 1H), 7.00 (d, J=6.0 Hz, 1H), 6.68 (s, 1H), 3.05 (t, J=5.6 Hz, 2H), 1.16-1.08 (m, 1H), 0.55-0.50 (m, 2H), 0.27 (q, J=4.8 Hz, 2H), exchangeable protons not observed; MS (ES+) m/z 325.1 (M+1), 327.1 (M+1).

### Example 231

### Synthesis of N-(6-chloropyridin-3-yl)-6-((1-(trifluoromethyl)cyclopropyl)methoxy)isoquinolin-1-amine formate

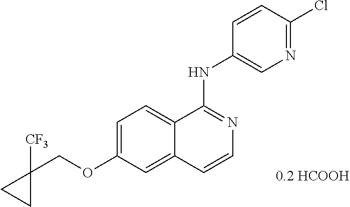 [SMILES: FC(F)(F)C1(COc2ccc3c(Nc4ccc(Cl)nc4)nccc3c2)CC1]

### Step 1. Preparation of 1-chloro-6-((1-(trifluoromethyl)cyclopropyl)methoxy)isoquinoline

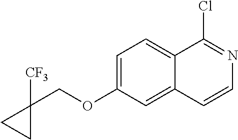 [SMILES: FC(F)(F)C1(COc2ccc3c(Cl)nccc3c2)CC1]

Following the procedure as described for EXAMPLE 1, Step 1 and making non-critical variations as required to replace 2-propanol with (1-(trifluoromethyl)cyclopropyl)methanol, the title compound was obtained as a colorless solid (0.070 g, 28% yield): MS (ES+) m/z 302.1 (M+1), 304.1 (M+1).

### Step 2. Preparation of N-(6-chloropyridin-3-yl)-6-((1-(trifluoromethyl)cyclopropyl)methoxy)isoquinolin-1-amine formate

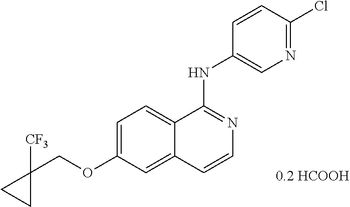 [SMILES: FC(F)(F)C1(COc2ccc3c(Nc4ccc(Cl)nc4)nccc3c2)CC1]

Following the procedure as described for EXAMPLE 230, step 3 and making variations as required to replace 1-chloro-N-(cyclopropylmethyl)isoquinolin-6-amine with 1-chloro-6-((1-(trifluoromethyl)cyclopropyl)methoxy)isoquinoline, the title compound was obtained as a colorless solid (0.0337 g, 32% yield): ¹H NMR (400 MHz, CD₃OD) δ 8.73-8.71 (m, 1H), 8.44 (s, 0.2H), 8.29-8.23 (m, 2H), 7.90 (d, J=5.9 Hz, 1H), 7.42-7.37 (m, 1H), 7.26-7.14 (m, 3H), 4.27 (s, 2H), 1.20-1.15 (m, 2H), 1.03-1.02 (m, 2H), exchangeable protons not observed; MS (ES+) m/z 394.1 (M+1), 396.1 (M+1).

### Example 232

### Synthesis of N¹-(6-chloropyridin-3-yl)-N⁶-(cyclopropylmethyl)-5-fluoroisoquinoline-1,6-diamine

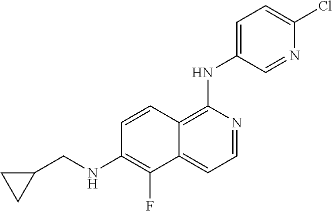 [SMILES: Fc1c(NCC2CC2)ccc2c(Nc3ccc(Cl)nc3)nccc12]

### Step 1. Preparation of 6-((cyclopropylmethyl)amino)-5-fluoroisoquinolin-1-ol

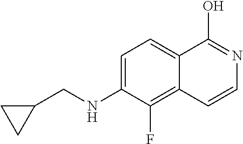 [SMILES: Oc1nccc2c(F)c(NCC3CC3)ccc12]

To a solution of 6-bromo-5-fluoroisoquinolin-1-ol (0.600 g, 2.48 mmol, prepared according to PCT Published Patent Application No. WO 2013/092756 A1) and cyclopropylmethanamine (0.353 g, 4.96 mmol) in dioxane (10 mL) was added sodium tert-butoxide (0.476 g, 4.96 mmol) and [(2-di-tert-butylphosphino-2′,4′,6′-triisopropyl-1,1′-biphenyl)-2-(2′-amino-1,1′-biphenyl)]palladium(II) methanesulfonate ((0.394 g, 0.496 mmol). The reaction mixture was stirred at 100° C. for 20 h. After cooling to ambient temperature, the reaction mixture was poured into saturated ammonium chloride solution (20 mL) and extracted with ethyl acetate (3×50 mL). The combined organic phase was washed with brine (20 mL), dried over anhydrous sodium sulfate, and filtered. Concentration of the filtrate under reduced pressure and purification of the obtained residue by silica gel column chromatography, eluting with a gradient of 30 to 45% of ethyl acetate in petroleum ether, afforded the title compound as a colorless solid (0.250 g, 37% yield): ¹H NMR (400 MHz, DMSO-d₆) δ 10.84 (br s, 1H), 7.81 (d, J=8.9 Hz, 1H), 7.07 (dd, J=7.2, 6.0 Hz, 1H), 6.97 (t, J=8.4 Hz, 1H), 6.40 (d, J=7.1 Hz, 1H), 6.24-6.20 (m, 1H), 3.11 (t, J=6.3 Hz, 2H), 1.13-1.06 (m, 1H), 0.48-0.43 (m, 2H), 0.27-0.23 (m, 2H); ¹⁹F NMR (376 MHz, DMSO-d₆) δ−147.6 (s).

### Step 2. Preparation of 1-chloro-N-(cyclopropylmethyl)-5-fluoroisoquinolin-6-amine

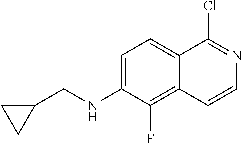 [SMILES: Fc1c(NCC2CC2)ccc2c(Cl)nccc12]

To a mixture of 6-((cyclopropylmethyl)amino)-5-fluoroisoquinolin-1-ol (0.050 mg, 0.215 mmol) in phosphoryl chloride (1.65 g, 10.8 mmol) was added dropwise triethylamine (0.022 mg, 0.215 mmol). The mixture was stirred at 85° C. for 4 h. After cooling to ambient temperature, the reaction mixture was concentrated under reduced pressure. To the obtained residue was then added saturated sodium bicarbonate solution (10 mL), and the mixture was extracted with ethyl acetate (3×10 mL). The combined organic phase was washed with brine (10 mL), dried over anhydrous sodium sulfate, and filtered. Concentration of the filtrate under pressure and purification of the obtained residue by preparative thin layer chromatography, eluting with 25% of ethyl acetate in petroleum ether, afforded the title compound as a yellowish solid (0.030 g, 56% yield): ¹H NMR (400 MHz, CDCl₃) δ 8.10 (d, J=6.0 Hz, 1H), 7.98 (d, J=9.2 Hz, 1H), 7.56 (d, J=5.6 Hz, 1H), 7.22-7.09 (m, 1H), 4.94-4.23 (m, 1H), 3.18 (d, J=6.8 Hz, 2H), 1.21-1.09 (m, 1H), 0.68-0.58 (m, 2H), 0.37-0.28 (m, 2H); ¹⁹F NMR (376 MHz, CDCl₃) δ−150.3 (s).

### Step 3. Preparation of N¹-(6-chloropyridin-3-yl)-N⁶-(cyclopropylmethyl)-5-fluoroisoquinoline-1,6-diamine

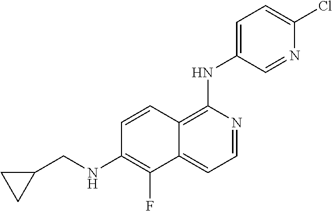 [SMILES: Fc1c(NCC2CC2)ccc2c(Nc3ccc(Cl)nc3)nccc12]

To a solution of 1-chloro-N-(cyclopropylmethyl)-5-fluoroisoquinolin-6-amine (0.100 mg, 0.399 mmol) and 6-chloropyridin-3-amine (0.056 g, 0.439 mmol) in N,N-dimethylacetamide (1 mL) was added a 4 M solution of hydrogen chloride in dioxane (0.110 mL, 0.440 mmol), and the reaction mixture was stirred at 90° C. for 5 h. After cooling to ambient temperature, the mixture was filtered. The obtained solid was washed with ethyl acetate (10 mL) and collected.

Purification of the residue by reverse-phase preparative HPLC (Phenomenex Gemini-NX C18 75 mm×30 mm, 3 μm column), eluting with a gradient of 15 to 45% of acetonitrile in water (containing 0.225% of formic acid), provided the title compound as a colorless solid (0.074 g, 51% yield): ¹H NMR (400 MHz, DMSO-d₆) δ9.31 (s, 1H), 8.86 (d, J=2.8 Hz, 1H), 8.47-8.36 (m, 1H), 8.18 (d, J=9.2 Hz, 1H), 7.89 (d, J=6.0 Hz, 1H), 7.43 (d, J=8.8 Hz, 1H), 7.27 (t, J=8.8 Hz, 1H), 7.08 (d, J=6.0 Hz, 1H), 6.19 (br s, 1H), 3.19 (t, J=6.4 Hz, 2H), 1.19-1.04 (m, 1H), 0.53-0.42 (m, 2H), 0.35-0.23 (m, 2H); ¹⁹F NMR (376 MHz, DMSO-d₆) δ−149.1 (s); MS (ES+) m/z 343.2 (M+1), 345.2 (M+1).

### Example 233

### Synthesis of 6-(cyclopropylmethoxy)-5-fluoro-N-(2-methylpyrimidin-5-yl)isoquinolin-1-amine

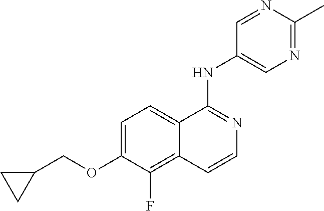 [SMILES: Cc1ncc(Nc2nccc3c(F)c(OCC4CC4)ccc23)cn1]

Following the procedure as described for EXAMPLE 24 and making variations as required to replace 1-chloro-6-(cyclopropylmethoxy)isoquinoline with 1-chloro-6-(cyclopropylmethoxy)-5-fluoroisoquinoline, the title compound was obtained as a colorless solid (0.032 g, 22% yield): ¹H NMR (400 MHz, DMSO-d₆) δ9.43 (s, 1H), 9.14 (s, 2H), 8.33 (d, J=9.2 Hz, 1H), 8.00 (d, J=6.0 Hz, 1H), 7.62 (t, J=8.8 Hz, 1H), 7.22 (d, J=6.0 Hz, 1H), 4.13 (d, J=7.2 Hz, 2H), 2.58 (s, 3H), 1.30 (s, 1H), 0.68-0.55 (m, 2H), 0.44-0.31 (m, 2H); ¹⁹F NMR (376 MHz, DMSO-d₆) δ−146.1 (s); MS (ES+) m/z 325.2 (M+1).

### Example 234

### Synthesis of 1-(((1-((6-chloropyridin-3-yl)amino)isoquinolin-6-yl)oxy)methyl)-3,3-difluorocyclobutane-1-carbonitrile

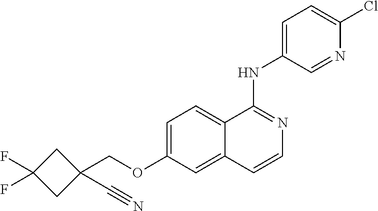 [SMILES: N#CC1(COc2ccc3c(Nc4ccc(Cl)nc4)nccc3c2)CC(F)(F)C1]

### Step 1. Preparation of 1-(chloromethyl)-3,3-difluorocyclobutane-1-carbonitrile

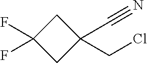

To a solution of 3,3-difluorocyclobutane-1-carbonitrile (0.200 g, 1.71 mmol) in tetrahydrofuran (10 mL) was added dropwise a 2 M solution of lithium diisopropylamide in tetrahydrofuran (1.02 mL, 2.04 mmol) at −60° C. and the reaction mixture was stirred at −60° C. for 1 h. To it was then added dropwise a solution of bromochloromethane (0.442 g, 3.42 mmol) in tetrahydrofuran (1 mL) at −60° C. The reaction mixture was allowed to warm to ambient temperature and stirred for 3 h. The reaction mixture was quenched with water (10 mL) and extracted with ethyl acetate (3×30 mL). The combined organic extracts were washed with brine (3×30 mL), dried over anhydrous sodium sulfate, and filtered. Concentration of the filtrate under reduced pressure provided the title compound as a brownish oil (0.170 g, 60% yield), which was used in the next step without further purification: ¹H NMR (400 MHz, CDCl₃) δ 3.79 (d, J=0.8 Hz, 2H), 3.24-3.14 (m, 2H), 2.94-2.84 (m, 2H).

### Step 2. Preparation of 1-(((1-chloroisoquinolin-6-yl)oxy)methyl)-3,3-difluorocyclobutane-1-carbonitrile

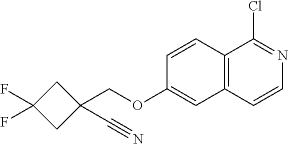 [SMILES: N#CC1(COc2ccc3c(Cl)nccc3c2)CC(F)(F)C1]

Following the procedure as described for EXAMPLE 192, Step 1, and making variations as required to replace 4-(chloromethyl)pyridine hydrochloride with 1-(chloromethyl)-3,3-difluorocyclobutane-1-carbonitrile, the title compound was obtained as a colorless solid (0.035 g, 17% yield): ¹H NMR (400 MHz, DMSO-d₆) δ 8.28-8.20 (m, 2H), 7.78 (d, J=5.6 Hz, 1H), 7.56 (d, J=2.4 Hz, 1H), 7.49 (dd, J=9.2, 2.4 Hz, 1H), 4.53 (s, 2H), 3.29-3.13 (m, 4H).

### Step 3. Preparation of 1-(((1-((6-chloropyridin-3-yl)amino)isoquinolin-6-yl)oxy)methyl)-3,3-difluorocyclobutane-1-carbonitrile

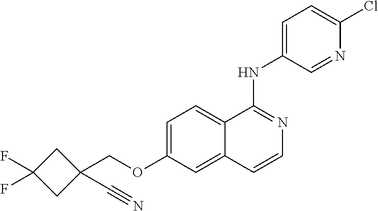 [SMILES: N#CC1(COc2ccc3c(Nc4ccc(Cl)nc4)nccc3c2)CC(F)(F)C1]

To a solution of 1-(((1-chloroisoquinolin-6-yl)oxy)methyl)-3,3-difluorocyclobutane-1-carbonitrile (0.035 g, 0.111 mmol), and 6-chloropyridin-3-amine (0.0175 mg, 0.136 mmol) in 2-methylbutan-2-ol (2 mL) was added (2-dicyclohexylphosphino-2′,6′-diisopropoxy-1,1′-biphenyl)[2-(2′-amino-1,1′-biphenyl)]palladium(II) methanesulfonate (0.0010 g, 0.0113 mmol) and cesium carbonate (0.111 g, 0.340 mmol). The reaction mixture was stirred at 100° C. in a microwave reactor for 2 h. After cooling to ambient temperature, the reaction mixture was diluted with water (10 mL) and extracted with ethyl acetate (3×10 mL). The combined organic extracts were washed with brine (3×10 mL), dried over anhydrous sodium sulfate, and filtered. The filtrate was concentrated under reduced pressure, and the obtained residue was purified by silica gel column chromatography, eluting with a gradient of 0 to 100% of ethyl acetate in petroleum ether. The desired fraction was collected and concentrated under reduced pressure, and the obtained residue was then purified by reverse-phase preparative HPLC (Phenomenex Luna C18 150 mm×25 mm, 10 μm column), eluting with a gradient of 17 to 47% of acetonitrile in water (containing formic acid) to provide the title compound as a colorless solid (0.013 g, 29% yield): ¹H NMR (400 MHz, DMSO-d₆) δ9.42 (s, 1H), 8.89 (d, J=2.8 Hz, 1H), 8.52-8.47 (m, 1H), 8.43 (dd, J=8.8, 2.8 Hz, 1H), 7.99 (d, J=5.6 Hz, 1H), 7.45 (d, J=8.8 Hz, 1H), 7.38-7.33 (m, 2H), 7.19 (d, J=5.6 Hz, 1H), 4.49 (s, 2H), 3.29-3.10 (m, 4H); ¹⁹F NMR (376 MHz 376 MHz, DMSO-d₆) δ−84.8 (d, J=195.2 Hz, 1F), −89.4 (d, J=195.2 Hz, 1F); MS (ES+) m/z 401.1 (M+1), 403.1 (M+1).

### Example 235

### Synthesis of 3,3-difluoro-1-(((1-((2-methylpyrimidin-5-yl)amino)isoquinolin-6-yl)oxy)methyl)cyclobutane-1-carbonitrile formate

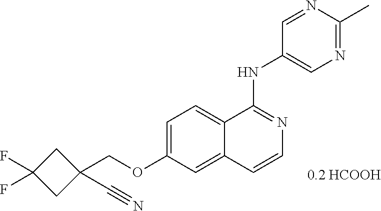 [SMILES: Cc1ncc(Nc2nccc3cc(OCC4(C#N)CC(F)(F)C4)ccc23)cn1]

Following the procedure as described for EXAMPLE 234, Step 3, and making variations as required to replace 6-chloropyridin-3-amine with 2-methylpyrimidin-5-amine, the title compound was obtained as a colorless solid (0.038 g, 26% yield): ¹H NMR (400 MHz, DMSO-d₆) δ 9.34 (s, 1H), 9.16 (s, 2H), 8.47 (d, J=10.0 Hz, 1H), 8.15 (s, 0.2H), 7.97 (d, J=5.6 Hz, 1H), 7.38-7.33 (m, 2H), 7.17 (d, J=5.6 Hz, 1H), 4.49 (s, 2H), 3.27-3.09 (m, 4H), 2.58 (s, 3H); ¹⁹F NMR (376 MHz, DMSO) δ−84.8 (d, J=195.5 Hz, 1F), −89.4 (d, J=195.3 Hz, 1F); MS (ES+) m/z 382.2 (M+1).

### Example 236

### Synthesis of N-(6-chloropyridin-3-yl)-6-(2-(2-methoxyethoxy)ethoxy)isoquinolin-1-amine

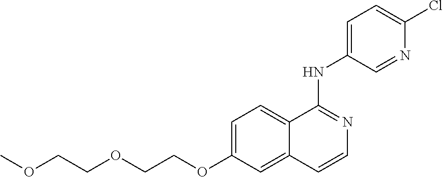 [SMILES: COCCOCCOc1ccc2c(Nc3ccc(Cl)nc3)nccc2c1]

### Step 1. Preparation of 1-chloro-6-(2-(2-methoxyethoxy)ethoxy)isoquinoline

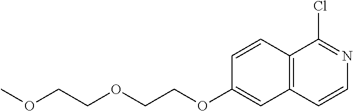 [SMILES: COCCOCCOc1ccc2c(Cl)nccc2c1]

To a solution of 1-chloroisoquinolin-6-ol (0.0250 g, 0.139 mmol), 2-(2-methoxyethoxy)ethan-1-ol (0.0500 g, 0.418 mmol), and triphenylphosphine (0.110 g, 0.418 mmol) in tetrahydrofuran (2 mL) was added diisopropyl azodicarboxylate (0.0840 g, 0.418 mmol) at 0° C. The reaction mixture was allowed to warm to ambient temperature and stirred for 16 h. To it was added ethyl acetate (5 mL) and water (5 mL) and the layers were separated. The aqueous phase was extracted with ethyl acetate (2×5 mL). The combined extracts were washed with brine (10 mL), dried over anhydrous sodium sulfate, and filtered. The filtrate was concentrated under reduced pressure and the obtained residue was purified by silica gel column chromatography, eluting with a gradient of 50% of ethyl acetate, to provide the title compound as a colorless oil (0.0550 g, quantitative yield): ¹H NMR (400 MHz, DMSO-d₆) δ8.21 (d, J=5.6 Hz, 1H), 8.17 (d, J=9.2 Hz, 1H), 7.75 (d, J=5.6 Hz, 1H), 7.53-7.49 (m, 1H), 7.44 (dd, J=9.2, 2.4 Hz, 1H), 4.31-4.26 (m, 2H), 3.85-3.79 (m, 2H), 3.62 (dd, J=5.6, 4.0 Hz, 2H), 3.45-3.38 (m, 2H), 3.25 (s, 3H).

### Step 2. Preparation of N-(6-chloropyridin-3-yl)-6-(2-(2-methoxyethoxy)ethoxy)isoquinolin-1-amine

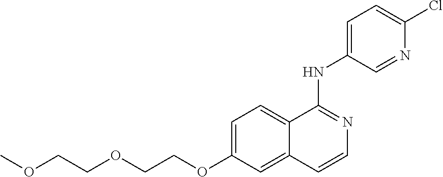 [SMILES: COCCOCCOc1ccc2c(Nc3ccc(Cl)nc3)nccc2c1]

To a solution of 1-chloro-6-(2-(2-methoxyethoxy)ethoxy)isoquinoline (0.055 g, 0.195 mmol) and 6-chloropyridin-3-amine (0.0300 g, 0.234 mmol) in propan-2-ol (4.00 mL) was added a 4 M solution of hydrogen chloride in dioxane (0.400 mL, 1.6 mmol). The reaction mixture was stirred at 70° C. for 4 h. After cooling to ambient temperature, the mixture was concentrated in vacuo. The obtained residue was purified by reverse-phase preparative HPLC (YMC Triart C18 150 mm×25 mm, 5 μm column), eluting with a gradient of 16 to 48% of acetonitrile in water (containing hydrochloric acid), to provide the title compound as a colorless solid (0.0078 g, 11% yield): ¹H NMR (400 MHz, DMSO-d₆) δ 8.81-8.70 (m, 1H), 8.64-8.54 (m, 1H), 8.27-8.14 (m, 1H), 7.82-7.61 (m, 2H), 7.52-7.43 (m, 2H), 7.28 (d, J=6.8 Hz, 1H), 4.35-4.29 (m, 2H), 3.86-3.80 (m, 2H), 3.61 (s, 2H), 3.50-3.48 (m, 2H), 3.25 (s, 3H), NH not observed; MS (ES+) m/z 374.1 (M+1), 376.1 (M+1).

### Example 237

### Synthesis of (S)—N-(6-chloropyridin-3-yl)-6-(1-(pyridin-4-yl)ethoxy)isoquinolin-1-amine

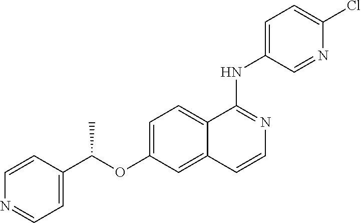 [SMILES: C[C@H](Oc1ccc2c(Nc3ccc(Cl)nc3)nccc2c1)c1ccncc1]

Following the procedure as described for EXAMPLE 236 and making variations as required to replace 2-(2-methoxyethoxy)ethan-1-ol with (R)-1-(pyridin-4-yl)ethan-1-ol, the title compound was obtained as a colorless solid (0.049 g, 13% yield): ¹H NMR (400 MHz, DMSO-d₆) d 9.34 (s, 1H), 8.85 (d, J=2.8 Hz, 1H), 8.56-8.54 (m, 2H), 8.44-8.37 (m, 2H), 7.91 (d, J=5.6 Hz, 1H), 7.47 (d, J=6.0 Hz, 2H), 7.43 (d, J=8.8 Hz, 1H), 7.35 (dd, J=9.2, 2.4 Hz, 1H), 7.18 (d, J=2.4 Hz, 1H), 7.06 (d, J=5.6 Hz, 1H), 5.82-5.76 (m, 1H), 1.63 (d, J=6.4 Hz, 3H); MS (ES+) m/z 377.0 (M+1), 379.0 (M+1).

### Example 238

### Synthesis of (1-(((1-((6-chloropyridin-3-yl)amino)isoquinolin-6-yl)oxy)methyl)cyclopropyl)methanol

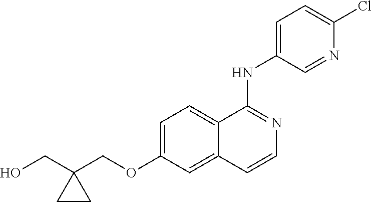 [SMILES: OCC1(COc2ccc3c(Nc4ccc(Cl)nc4)nccc3c2)CC1]

Following the procedure as described for EXAMPLE 236 and making variations as required to replace 2-(2-methoxyethoxy)ethan-1-ol with (1-(((tert-butyldiphenylsilyl)oxy)methyl)cyclopropyl)methanol, the title compound was obtained as a colorless solid (0.082 g, 14% yield): ¹H NMR (400 MHz, DMSO-d₆) δ9.36 (s, 1H), 8.89-8.87 (m, 1H), 8.44-8.41 (m, 2H), 7.96-7.94 (m, 1H), 7.46-7.42 (m, 1H), 7.31-7.26 (m, 2H), 7.18-7.15 (m, 1H), 4.69-4.65 (m, 1H), 4.03 (s, 2H), 3.44 (d, J=5.6 Hz, 2H), 0.57-0.53 (m, 4H); MS (ES+) m/z 356.2 (M+1), 358.2 (M+1).

### Example 239

### Synthesis of N-(6-chloropyridin-3-yl)-6-((4-fluorotetrahydro-2H-pyran-4-yl)methoxy)isoquinolin-1-amine

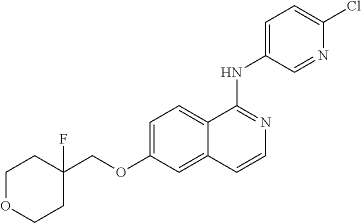 [SMILES: FC1(COc2ccc3c(Nc4ccc(Cl)nc4)nccc3c2)CCOCC1]

### Step 1. Preparation of (4-fluorotetrahydro-2H-pyran-4-yl) methyl 4-methyl benzenesulfonate

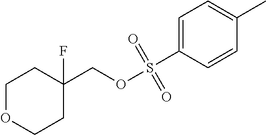 [SMILES: Cc1ccc(S(=O)(=O)OCC2(F)CCOCC2)cc1]

Following the procedure as described for EXAMPLE 36, Step 1, and making variations as required to replace 3-methyl-3-oxetanemethanol with (4-fluorotetrahydro-2H-pyran-4-yl)methanol, the title compound was obtained as a colorless solid (0.40 g, 93% yield): ¹H NMR (400 MHz, DMSO-d₆) δ 7.81 (d, J=8.4 Hz, 2H), 7.49 (d, J=8.4 Hz, 2H), 4.18-4.12 (m, 2H), 3.67 (td, J=11.6, 3.6 Hz, 2H), 3.48 (dt, J=10.8, 4.0 Hz, 2H), 2.43 (s, 3H), 1.75-1.56 (m, 4H).

### Step 2. Preparation of 1-chloro-6-((4-fluorotetrahydro-2H-pyran-4-yl)methoxy)isoquinoline

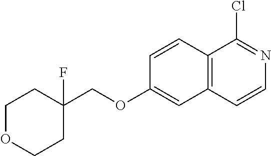 [SMILES: FC1(COc2ccc3c(Cl)nccc3c2)CCOCC1]

Following the procedure as described for EXAMPLE 36, Step 2, and making variations as required to replace (3-methyloxetan-3-yl)methyl 4-methylbenzenesulfonate with (4-fluorotetrahydro-2H-pyran-4-yl)methyl 4-methylbenzenesulfonate, the title compound was obtained as a colorless solid (0.10 g, 50% yield): ¹H NMR (400 MHz, DMSO-d₆) δ8.23-8.17 (m, 2H), 7.76 (d, J=5.6 Hz, 1H), 7.54 (d, J=2.4 Hz, 1H), 7.47 (dd, J=9.2, 2.4 Hz, 1H), 4.34-4.28 (m, 2H), 3.79 (td, J=11.6, 3.6 Hz, 2H), 3.62 (dt, J=10.8, 4.0 Hz, 2H), 1.95-1.84 (m, 4H).

### Step 3. Preparation of N-(6-chloropyridin-3-yl)-6-((4-fluorotetrahydro-2H-pyran-4-yl)methoxy)isoquinolin-1-amine

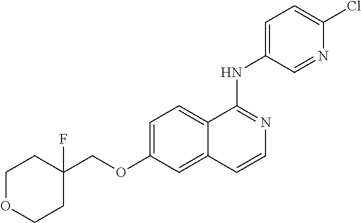 [SMILES: FC1(COc2ccc3c(Nc4ccc(Cl)nc4)nccc3c2)CCOCC1]

Following the procedure as described for EXAMPLE 236, Step 2, and making variations as required to replace 1-chloro-6-(2-(2-methoxyethoxy)ethoxy)isoquinoline with 1-chloro-6-((4-fluorotetrahydro-2H-pyran-4-yl)methoxy)isoquinoline, the title compound was obtained as a colorless solid (0.020 g, 30% yield): ¹H NMR (400 MHz, DMSO-d₆) δ 9.39 (s, 1H), 8.88 (d, J=2.4 Hz, 1H), 8.47-8.41 (m, 2H), 7.97 (d, J=5.6 Hz, 1H), 7.45 (d, J=8.8 Hz, 1H), 7.34-7.31 (m, 2H), 7.18 (d, J=5.6 Hz, 1H), 4.30-4.24 (m, 2H), 3.80 (td, J=11.6, 3.6 Hz, 2H), 3.66-3.59 (m, 2H), 1.96-1.85 (m, 4H); ¹⁹F NMR (376 MHz, DMSO-d₆) δ−161.9 (s); MS (ES+) m/z 388.0 (M+1), 390.0 (M+1).

### Example 240

### Synthesis of 3-((1-((6-chloropyridin-3-yl)amino)isoquinolin-6-yl)oxy)-1,1,1-trifluoropropan-2-ol

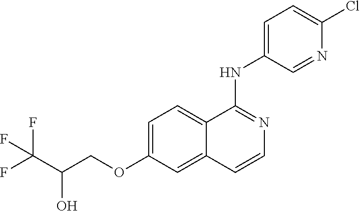 [SMILES: OC(COc1ccc2c(Nc3ccc(Cl)nc3)nccc2c1)C(F)(F)F]

A solution of 1-((6-chloropyridin-3-yl)amino)isoquinolin-6-ol (0.050 g, 0.184 mmol), 2-(trifluoromethyl)oxirane (0.0410 g, 0.368 mmol), and cesium carbonate (0.179 g, 0.552 mmol) in acetonitrile (2 mL) was stirred at 80° C. for 12 h. After cooling to ambient temperature, the was concentrated under reduced pressure. The obtained residue was purified by reverse-phase preparative HPLC (Phenomenex Luna C18 150 mm×25 mm, 10 μm column), eluting with a gradient of 12 to 42% of acetonitrile in water (containing formic acid), to give the title compound as an off-white solid (0.0160 g, 22% yield): ¹H NMR (400 MHz, DMSO-d₆) δ9.39 (s, 1H), 8.91-8.85 (m, 1H), 8.51-8.34 (m, 2H), 7.97 (d, J=5.6 Hz, 1H), 7.45 (d, J=8.8 Hz, 1H), 7.38 (d, J=2.8 Hz, 1H), 7.30 (dd, J=9.2, 2.8 Hz, 1H), 7.20 (d, J=6.0 Hz, 1H), 6.79-6.70 (m, 1H), 4.50-4.43 (m, 1H), 4.40-4.31 (m, 1H), 4.30-4.19 (m, 1H); ¹⁹F NMR (376 MHz, DMSO-d₆) δ−76.0 (s); MS (ES+) m/z 384.1 (M+1), 386.1 (M+1).

### Example 241

### Synthesis of 6-(isoxazol-4-ylmethoxy)-N-(2-methylpyrimidin-5-yl)isoquinolin-1-amine

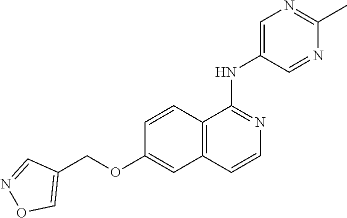 [SMILES: Cc1ncc(Nc2nccc3cc(OCc4cnoc4)ccc23)cn1]

### Step 1. Preparation of 1-((2-methylpyrimidin-5-yl)amino)isoquinolin-6-ol

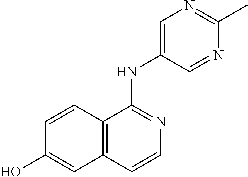 [SMILES: Cc1ncc(Nc2nccc3cc(O)ccc23)cn1]

To a solution of 1-chloroisoquinolin-6-ol (0.135 g, 0.752 mmol) in 1,4-dioxane (6.5 mL) was added 2-methylpyrimidin-5-amine (0.086 g, 0.789 mmol), tris(dibenzylideneacetone)dipalladium(0) (0.0688 g, 0.075 mmol), 2-dicyclohexylphosphino-2′,6′-dimethoxy-1,1′-biphenyl (0.062 g, 0.150 mmol), and potassium phosphate tribasic (0.638 g, 3.01 mmol). The reaction mixture was degassed by passing a stream of nitrogen through it for 10 minutes and then heated to 110° C. for 1 h. After cooling to ambient temperature, the reaction mixture was filtered. The filter cake was rinsed with ethyl acetate (15 mL) and the combined filtrate was concentrated in vacuo. The residue was purified by silica gel column chromatography, eluting with a gradient of 0 to 20% of methanol in dichloromethane, to afford the title compound was obtained as a colorless solid (0.160 g, 84% yield): ¹H NMR (400 MHz, DMSO-d₆) δ 10.29 (s, 1H), 9.22 (s, 1H), 9.14 (s, 2H), 8.35 (d, J=9.2 Hz, 1H), 7.86 (d, J=5.8 Hz, 1H), 7.14 (dd, J=9.1, 2.5 Hz, 1H), 7.05 (m, 2H), 2.57 (s, 3H); MS (ES+) m/z 253.2 (M+1).

### Step 2. Preparation of 6-(isoxazol-4-ylmethoxy)-N-(2-methylpyrimidin-5-yl)isoquinolin-1-amine

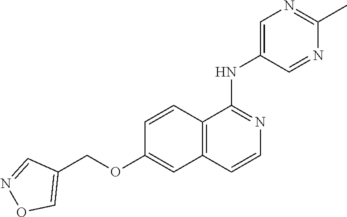 [SMILES: Cc1ncc(Nc2nccc3cc(OCc4cnoc4)ccc23)cn1]

A solution of 1-((2-methylpyrimidin-5-yl)amino)isoquinolin-6-ol (0.0500 g, 0.198 mmol), isoxazol-4-ylmethanol (0.0236 g, 0.238 mmol), and triphenylphosphine (0.0624 g, 0.238 mmol) in tetrahydrofuran (1 mL) was stirred at ambient temperature for 10 minutes. The mixture was then cooled to 0° C., and diethyl azodicarboxylate (0.037 mL, 0.238 mmol) was added dropwise to it. The reaction mixture was allowed to warm to ambient temperature and stirred for 2 h. To the mixture was then added saturated sodium bicarbonate solution (15 mL) and the mixture was extracted with ethyl acetate (3×15 mL). The combined organic layers were washed with brine (2×10 mL), dried over anhydrous sodium sulfate, and concentrated in vacuo. The obtained residue was purified by silica gel column chromatography, eluting with a gradient of 0 to 20% of methanol in dichloromethane. The residue was then purified by reverse-phase column chromatography, eluting with a gradient of 5 to 40% of acetonitrile in water (containing 0.5% of formic acid), to provide the title compound as a colorless solid (0.0060 g, 9% yield): ¹H NMR (400 MHz, DMSO-d₆) δ 9.32 (s, 1H), 9.16 (s, 1H), 9.15 (bs, 2H), 8.83 (s, 1H), 8.44 (d, J=9.3 Hz, 1H), 7.96 (d, J=5.8 Hz, 1H), 7.42 (d, J=2.6 Hz, 1H), 7.32 (dd, J=9.2, 2.6 Hz, 1H), 7.19 (d, J=5.8 Hz, 1H), 5.20 (s, 2H), 2.57 (s, 3H); MS (ES+) m/z 334.0 (M+1).

### Example 242

### Synthesis of N-(2-chloropyrimidin-5-yl)-6-(isoxazol-4-ylmethoxy)isoquinolin-1-amine

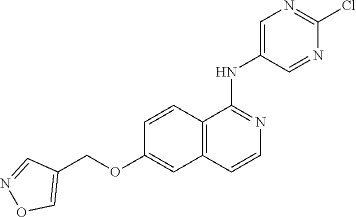 [SMILES: Clc1ncc(Nc2nccc3cc(OCc4cnoc4)ccc23)cn1]

### Step 1. Preparation of 4-(((1-chloroisoquinolin-6-yl)oxy)methyl)isoxazole

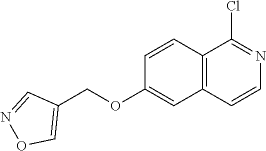 [SMILES: Clc1nccc2cc(OCc3cnoc3)ccc12]

A solution of 1-chloroisoquinolin-6-ol (0.220 g, 1.23 mmol), isoxazol-4-ylmethanol (0.146 g, 1.47 mmol) and triphenylphosphine (0.386 g, 1.47 mmol) in tetrahydrofuran (4.7 mL) was stirred at ambient temperature for 10 minutes. The mixture was then cooled to 0° C. and diethyl azodicarboxylate (0.231 mL, 1.47 mmol) was added dropwise to it. The reaction was allowed to warm to ambient temperature and stirred for 2 h. To the mixture was then added saturated sodium bicarbonate solution (20 mL) and the mixture was extracted with ethyl acetate (3×20 mL). The combined organic layers were washed with brine (2×20 mL), dried over anhydrous sodium sulfate, and concentrated in vacuo. The obtained residue was purified by silica gel column chromatography, eluting with a gradient of 5 to 100% of ethyl acetate in heptane, to provide the title compound as a colorless solid (0.320 g, quantitative yield): MS (ES+) m/z 261.2 (M+1), 263.2 (M+1).

### Step 2. Preparation of N-(2-chloropyrimidin-5-yl)-6-(isoxazol-4-ylmethoxy)isoquinolin-1-amine

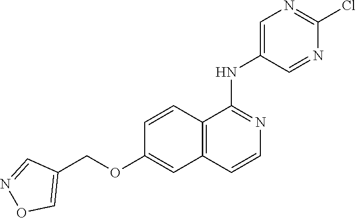 [SMILES: Clc1ncc(Nc2nccc3cc(OCc4cnoc4)ccc23)cn1]

Following the procedure as described for EXAMPLE 207, Step 3 and making variations as required to replace 1-(((1-chloroisoquinolin-6-yl)oxy)methyl)cyclopropane-1-carbonitrile with 4-(((1-chloroisoquinolin-6-yl)oxy)methyl)isoxazole, the title compound was obtained as a colorless solid (0.022 g, 11% yield): ¹H NMR (400 MHz, DMSO-d₆) δ9.59 (s, 1H), 9.29 (s, 2H), 9.17 (s, 1H), 8.83 (s, 1H), 8.44 (d, J=9.3 Hz, 1H), 8.02 (d, J=5.8 Hz, 1H), 7.46 (d, J=2.5 Hz, 1H), 7.35 (dd, J=9.2, 2.6 Hz, 1H), 7.27 (d, J=5.8 Hz, 1H), 5.21 (s, 2H); MS (ES+) m/z 354.0 (M+1), 356.2 (M+1).

### Example 243

### Synthesis of 1-((2-chloropyrimidin-5-yl)amino)isoquinolin-6-ol

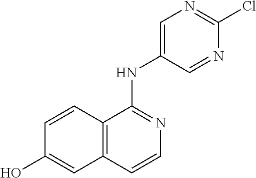 [SMILES: Oc1ccc2c(Nc3cnc(Cl)nc3)nccc2c1]

### Step 1. Preparation of N-(2-chloropyrimidin-5-yl)-6-methoxyisoquinolin-1-amine

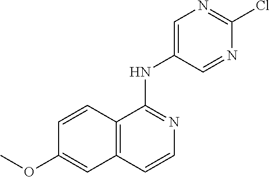 [SMILES: COc1ccc2c(Nc3cnc(Cl)nc3)nccc2c1]

Following the procedure as described for EXAMPLE 207, Step 3 and making variations as required to replace 1-(((1-chloroisoquinolin-6-yl)oxy)methyl)cyclopropane-1-carbonitrile with 1-chloro-6-methoxyisoquinoline, the title compound was obtained as a colorless solid (0.530 g, 29% yield): MS (ES+) m/z 287.6 (M+1), 289.6 (M+1).

### Step 2. Preparation of 1-((2-chloropyrimidin-5-yl)amino)isoquinolin-6-ol

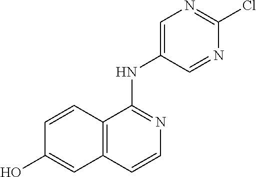 [SMILES: Oc1ccc2c(Nc3cnc(Cl)nc3)nccc2c1]

To a solution of N-(2-chloropyrimidin-5-yl)-6-methoxyisoquinolin-1-amine (0.150 g, 0.523 mmol) in dichloromethane (9 mL) was added dropwise boron tribromide (0.403 mL, 4.19 mmol, 8.0 eq) at 0° C. The reaction mixture was allowed to warm to ambient temperature and stirred for 16 h. The reaction mixture was then poured into iced water, stirred for 15 minutes, and carefully neutralized by addition of saturated sodium bicarbonate solution (20 mL). The aqueous phase was extracted with dichloromethane (2×30 mL). The combined organic phase was dried over anhydrous sodium sulfate and filtered. The filtrate was concentrated in vacuo and the obtained residue was purified by silica gel column chromatography, eluting with a gradient of 0 to 20% of methanol in dichloromethane. The obtained residue was then purified by reverse-phase preparative HPLC (Phenomenex Gemini-NX C18 150 mm×30 mm, 5 μm column), eluting with a gradient of 10 to 30% of acetonitrile in water (containing 0.5% of formic acid), to give the title compound as a colorless solid (0.009 g, 6% yield): ¹H NMR (400 MHz, DMSO-d₆) δ 10.38 (s, 1H), 9.51 (s, 1H), 9.28 (s, 2H), 8.35 (d, J=9.2 Hz, 1H), 7.92 (d, J=5.8 Hz, 1H), 7.18 (dd, J=9.1, 2.5 Hz, 1H), 7.13 (d, J=5.8 Hz, 1H), 7.07 (d, J=2.4 Hz, 1H); MS (ES+) m/z 273.0 (M+1), 275.0 (M+1).

### Example 244

### Synthesis of N′-(6-chloropyridin-3-yl)-N⁶-((1-methyl-1H-pyrazol-4-yl)methyl)isoquinoline-1,6-diamine

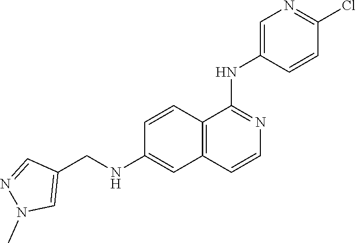 [SMILES: Cn1cc(CNc2ccc3c(Nc4ccc(Cl)nc4)nccc3c2)cn1]

A solution of N-(6-chloro-3-pyridyl)-6-fluoro-N-(2-trimethylsilylethoxymethyl)isoquinolin-1-amine (0.030 g, 0.074 mmol) and (1-methyl-1H-pyrazol-4-yl)methanamine (0.047 mL, 0.371 mmol) in dimethyl sulfoxide (0.74 mL) was stirred at 140° C. for 24 h. After cooling to ambient temperature, the reaction mixture was diluted with water (20 mL) and extracted with ethyl acetate (3×20 mL). The combined organic layers were dried over anhydrous sodium sulfate, filtered, and the filtrate was concentrated in vacuo. The residue was purified by silica gel column chromatography, eluting with a gradient of 0 to 100% of ethyl acetate in heptane, to afford N′-(6-chloropyridin-3-yl)-N⁶-((1-methyl-1H-pyrazol-4-yl)methyl)isoquinoline-1,6-diamine as a colorless solid (0.014 g, 44% yield): ¹H NMR (400 MHz, CDCl₃) δ8.43 (d, J=2.8 Hz, 1H), 8.32 (dd, J=8.7, 2.9 Hz, 1H), 7.92 (d, J=5.9 Hz, 1H), 7.72 (d, J=9.0 Hz, 1H), 7.51 (s, 1H), 7.37 (s, 1H), 7.29 (s, 1H), 7.00 (d, J=5.9 Hz, 1H), 6.89 (dd, J=9.0, 2.4 Hz, 1H), 6.73 (d, J=2.3 Hz, 1H), 4.30 (s, 2H), 3.89 (s, 3H), (NH not observed); MS (ES+) m/z 365.2 (M+1), 367.2 (M+1).

### Example 245

### Synthesis of N′-(6-chloropyridin-3-yl)-N⁶-((3-methyloxetan-3-yl)methyl)isoquinoline-1,6-diamine

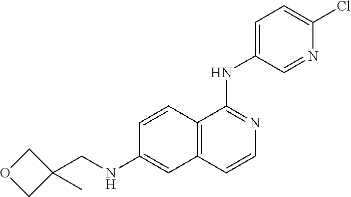 [SMILES: CC1(CNc2ccc3c(Nc4ccc(Cl)nc4)nccc3c2)COC1]

Following the procedure as described for EXAMPLE 244 and making variations as required to replace (1-methyl-1H-pyrazol-4-yl)methanamine with (3-methyloxetan-3-yl)methanamine, the title compound was obtained as a colorless solid (0.007 g, 14% yield): ¹H NMR (400 MHz, DMSO-d₆) δ 9.14 (s, 1H), 8.85 (d, J=2.7 Hz, 1H), 8.42 (dd, J=8.7, 2.9 Hz, 1H), 8.20-8.18 (m, 1H), 7.80 (d, J=5.8 Hz, 1H), 7.41 (d, J=8.6 Hz, 1H), 7.10 (dd, J=9.2, 2.4 Hz, 1H), 6.96 (d, J=6.0 Hz, 1H), 6.71 (d, J=2.3 Hz, 1H), 6.44 (t, J=5.6 Hz, 1H), 4.45 (d, J=5.8 Hz, 2H), 4.28 (d, J=5.8 Hz, 2H), 3.37 (s, 2H), 1.37 (s, 3H); MS (ES+) m/z 355.2 (M+1), 357.2 (M+1).

### Example 246

### Synthesis of (E)-3-(1-((6-chloropyridin-3-yl)amino)isoquinolin-6-yl)-N,N-dimethylacrylamide

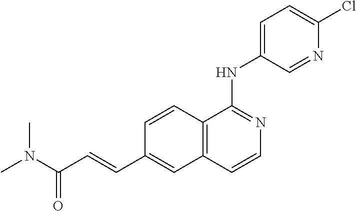 [SMILES: CN(C)C(=O)/C=C/c1ccc2c(Nc3ccc(Cl)nc3)nccc2c1]

To a solution of 6-bromo-N-(6-chloropyridin-3-yl)isoquinolin-1-amine (0.200 g, 0.598 mmol), N,N-dimethylprop-2-enamide (0.119 g, 1.20 mmol), tris(o-tolyl)phosphine (0.073 g, 0.239 mmol), and N,N-diisopropylethylamine (0.232 g, 1.79 mmol) in N,N-dimethylformamide (3 mL) was added palladium(II) acetate(26.8 mg, 0.120 mmol) and the mixture was stirred at 100° C. for 16 h. After cooling to ambient temperature, the mixture was filtered through a pad of celite. The filter cake was washed with a dichloromethane/methanol (1/1, 30 mL), and the combined filtrate was concentrated in vacuo. Purification of the residue by reverse-phase preparative HPLC, eluting with a gradient of 37 to 75% of acetonitrile in water (containing 10 mM of ammonium bicarbonate) as eluent, afforded the title compound as a colorless solid (0.150 g, 68% yield): ¹H NMR (500 MHz, DMSO-d₆) δ 9.49 (s, 1H), 8.90 (d, J=2.8 Hz, 1H), 8.52 (d, J=8.8 Hz, 1H), 8.43 (dd, J=8.7, 2.8 Hz, 1H), 8.11 (s, 1H), 8.08-8.00 (m, 2H), 7.61 (d, J=15.4 Hz, 1H), 7.48-7.46 (m, 1H), 7.46-7.40 (m, 1H), 7.26 (d, J=5.8 Hz, 1H), 3.22 (s, 3H), 2.96 (s, 3H); MS (ES+) m/z 353.2 (M+1), 355.1 (M+1).

### Example 247

### Synthesis of N-(6-chloropyridin-3-yl)-7-fluoro-6-((1-fluorocyclopropyl)methoxy)isoquinolin-1-amine

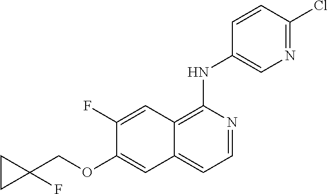 [SMILES: Fc1cc2c(Nc3ccc(Cl)nc3)nccc2cc1OCC1(F)CC1]

### Step 1. Preparation of N-(6-chloropyridin-3-yl)-6,7-difluoroisoquinolin-1-amine

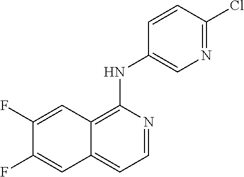 [SMILES: Fc1cc2ccnc(Nc3ccc(Cl)nc3)c2cc1F]

A mixture of 1-chloro-6,7-difluoro-isoquinoline (0.400 g, 2.00 mmol), 6-chloropyridin-3-amine (0.309 g, 2.40 mmol), 2-dicyclohexylphosphino-2′,6′-dimethoxybiphenyl (0.164 g, 0.401 mmol) and potassium phosphate tribasic (0.851 g, 4.01 mmol) in toluene (5 mL) was degassed and purged with nitrogen, and then tris(dibenzylideneacetone)dipalladium(0) (0.184 g, 0.200 mmol) was added to it. The reaction mixture was stirred at 100° C. for 16 h. After cooling to ambient temperature, the mixture was poured into ice water (5 mL) and extracted with ethyl acetate (3×2 mL). The combined organic phase was washed with brine (2 mL), dried over anhydrous sodium sulfate, and filtered. The filtrate was concentrated in vacuo and the obtained residue was purified by silica gel column chromatography, eluting with a gradient of 2 to 33% of ethyl acetate in petroleum ether, to give the title compound as a yellowish solid (0.380 g, 65% yield): ¹H NMR (400 MHz, DMSO-d₆) δ 9.41 (s, 1H), 8.86 (d, J=2.8 Hz, 1H), 8.62 (dd, J=12.4, 8.0 Hz, 1H), 8.39 (dd, J=8.8, 2.8 Hz, 1H), 8.06 (d, J=5.6 Hz, 1H), 7.98 (dd, J=11.2, 8.0 Hz, 1H), 7.48 (d, J=8.8 Hz, 1H), 7.29 (d, J=5.6 Hz, 1H).

### Step 2. Preparation of N-(6-chloropyridin-3-yl)-7-fluoro-6-((1-fluorocyclopropyl)methoxy)isoquinolin-1-amine

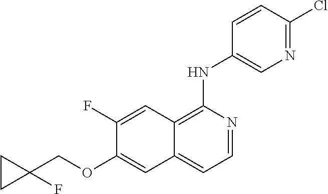 [SMILES: Fc1cc2c(Nc3ccc(Cl)nc3)nccc2cc1OCC1(F)CC1]

To a solution of N-(6-chloropyridin-3-yl)-6,7-difluoroisoquinolin-1-amine (0.100 g, 0.343 mmol) and (1-fluorocyclopropyl)methanol (0.093 g, 1.03 mmol) in 2-methyltetrahydrofuran (5 mL) was added potassium tert-butoxide (0.077 g, 0.686 mmol) and the mixture was stirred at 80° C. for 18 h. The mixture was poured into ice water (5 mL) and extracted with ethyl acetate (3×3 mL). The combined organic phase was washed with brine (3 mL), dried over anhydrous sodium sulfate, and filtered. The filtrate was concentrated in vacuo and the obtained residue was reverse-phase preparative HPLC (Waters XBridge OBD C18 150×40 mm, 10 μm column), eluting with a gradient of 30 to 65% of acetonitrile in water (containing 10 mM of ammonium bicarbonate), to yield the title compound as a colorless solid (0.032 g, 25% yield): ¹H NMR (400 MHz, CD₃OD) δ 8.74 (d, J=2.8 Hz, 1H), 8.28 (dd, J=8.8, 2.8 Hz, 1H), 8.14 (d, J=12.4 Hz, 1H), 7.93 (d, J=6.0 Hz, 1H), 7.40 (dd, J=8.4, 4.4 Hz, 2H), 7.19 (d, J=6.0 Hz, 1H), 4.57-4.43 (m, 2H), 1.27-1.15 (m, 2H), 0.80-0.95 (m, 2H), NH not observed; ¹H NMR (376 MHz 376 MHz, CD₃OD) δ−131.6 (s, 1F), −185.5 (s, 1F); MS (ES+) m/z 362.1 (M+1), 364.1 (M+1).

### Example 248

### Synthesis of 6-(((1H-pyrazol-4-yl)amino)methyl)-N-(6-chloropyridin-3-yl)isoquinolin-1-amine

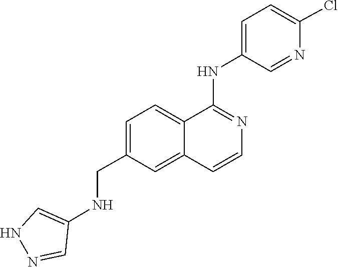 [SMILES: Clc1ccc(Nc2nccc3cc(CNc4cn[nH]c4)ccc23)cn1]

### Step 1. Preparation of 1-((6-chloropyridin-3-yl)amino)isoquinoline-6-carbaldehyde

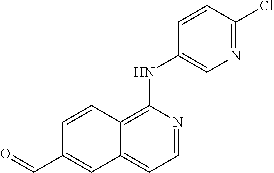 [SMILES: O=Cc1ccc2c(Nc3ccc(Cl)nc3)nccc2c1]

Following the procedure as described for EXAMPLE 247, Step 1, and making variations as required to replace 1-chloro-6,7-difluoro-isoquinoline with 1-chloroisoquinoline-6-carbaldehyde, the title compound was obtained as a brownish solid (0.270 g, 61% yield).

### Step 2. Preparation of 6-(((1H-pyrazol-4-yl)amino)methyl)-N-(6-chloropyridin-3-yl)isoquinolin-1-amine

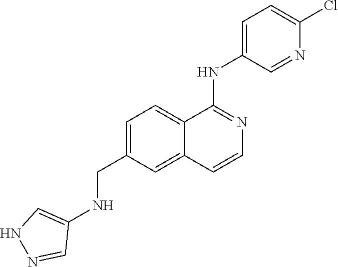 [SMILES: Clc1ccc(Nc2nccc3cc(CNc4cn[nH]c4)ccc23)cn1]

To a solution of 1-((6-chloropyridin-3-yl)amino)isoquinoline-6-carbaldehyde (0.200 g, 0.705 mmol) and 1H-pyrazol-4-amine (0.070 g, 0.846 mmol) in methanol (5 mL) was added acetic acid (0.085 g, 1.41 mmol) and the mixture was stirred at ambient temperature for 30 minutes. To it was then added sodium cyanoborohydride (0.089 g, 1.41 mmol) and the mixture was stirred at ambient temperature for 2 h. The mixture was concentrated under reduce pressure and the obtained residue was purified by reverse-phase preparative HPLC (Waters XBridge OBD C18 150×40 mm, 10 μm column), eluting with a gradient of 20 to 55% of acetonitrile in water (containing 10 mM of ammonium bicarbonate), to yield the title compound as a colorless solid (0.022 g, 9% yield): ¹H NMR (400 MHz, DMSO-d₆) δ12.01 (s, 1H), 9.42 (s, 1H), 8.89 (d, J=2.8 Hz, 1H), 8.41-8.48 (m, 2H), 8.00 (d, J=5.6 Hz, 1H), 7.81 (s, 1H), 7.67 (d, J=8.8 Hz, 1H), 7.45 (d, J=8.8 Hz, 1H), 7.22 (d, J=5.6 Hz, 1H), 7.04 (s, 2H), 5.11 (t, J=6.4 Hz, 1H), 4.25 (d, J=6.4 Hz, 2H); MS (ES+) m/z 351.1 (M+1), 353.1 (M+1).

### Example 249

### Synthesis of N-(6-chloropyridin-3-yl)-6-(((1-methyl-1H-pyrazol-4-yl)oxy)methyl)isoquinolin-1-amine

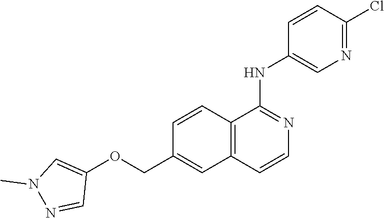 [SMILES: Cn1cc(OCc2ccc3c(Nc4ccc(Cl)nc4)nccc3c2)cn1]

### Step 1. Preparation of (1-((6-chloropyridin-3-yl)amino)isoquinolin-6-yl)methanol

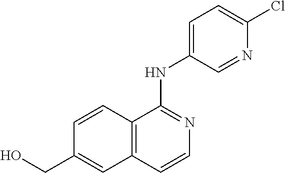 [SMILES: OCc1ccc2c(Nc3ccc(Cl)nc3)nccc2c1]

To a solution of 1-((6-chloropyridin-3-yl)amino)isoquinoline-6-carbaldehyde (0.300 g, 1.06 mmol) in tetrahydrofuran (1 mL) and methanol (1 mL) was added sodium borohydride (0.190 g, 5.02 mmol) at 0° C., and the mixture was stirred at 0° C. for 2 h. To the mixture was then added 2 M hydrochloric acid (0.5 mL) and the mixture was concentrated in vacuo. The obtained residue was purified by preparative thin layer chromatography, eluting with 10% of methanol in dichloromethane, to provide the title compound as a yellowish solid (0.120 g, 40% yield).

### Step 2. Preparation of N-(6-chloropyridin-3-yl)-6-(((1-methyl-1H-pyrazol-4-yl)oxy)methyl)isoquinolin-1-amine

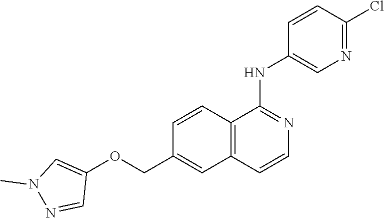 [SMILES: Cn1cc(OCc2ccc3c(Nc4ccc(Cl)nc4)nccc3c2)cn1]

To a mixture of (1-((6-chloropyridin-3-yl)amino)isoquinolin-6-yl)methanol (0.100 g, 0.349 mmol) and 1-methylpyrazol-4-ol (0.052 g, 0.524 mmol) in toluene (5 mL) was added 1,1′-(azodicarbonyl)dipiperidine (0.132 g, 0.524 mmol) followed by tributylphosphine (0.106 g, 0.524 mmol), and the mixture was stirred at 50° C. for 2 h. After cooling to ambient temperature, the mixture was poured into water (20 mL). The mixture was extracted with dichloromethane (3×5 mL). The combined organic phase was washed with brine (10 mL), dried over anhydrous sodium sulfate, and filtered. The filtrate was concentration in vacuo and the obtained residue was purified by reverse-phase preparative HPLC (Waters XBridge OBD C18 150×40 mm, 10 μm column), eluting with a gradient of 30 to 60% of acetonitrile in water (containing 10 mM of ammonium bicarbonate), to yield the title compound as a colorless solid (0.018 g, 14% yield): ¹H NMR (400 MHz, CDCl₃) δ 8.55 (d, J=2.8 Hz, 1H), 8.39 (dd, J=8.8, 2.8 Hz, 1H), 8.09 (d, J=5.6 Hz, 1H), 7.98 (d, J=8.4 Hz, 1H), 7.81 (s, 1H), 7.63 (dd, J=8.4, 1.2 Hz, 1H), 7.36-7.27 (m, 3H), 7.22 (d, J=5.6 Hz, 1H), 7.09 (s, 1H), 5.12 (s, 2H), 3.82 (s, 3H); MS (ES+) m/z 366.1 (M+1), 368.1 (M+1).

### Example 250

### Synthesis of N-(6-chloropyridin-3-yl)-6-((methyl(1H-pyrazol-4-yl)amino)methyl)isoquinolin-1-amine

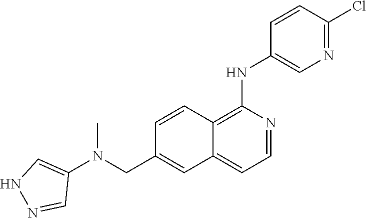 [SMILES: CN(Cc1ccc2c(Nc3ccc(Cl)nc3)nccc2c1)c1cn[nH]c1]

### Step 1. Preparation of N-methyl-1H-pyrazol-4-amine

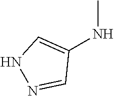

To a lithium aluminium hydride (2.5 M in tetrahydrofuran, 2.18 mL, 5.45 mmol) in tetrahydrofuran (5 mL) was added a solution of tert-butyl (1H-pyrazol-4-yl)carbamate (0.500 g, 2.73 mmol) in tetrahydrofuran (5 mL) dropwise at 0° C. The mixture was allowed to warm to ambient temperature and stirred for 1 h, and then heated to 70° C. for 12 h. After cooling to ambient temperature, the mixture was quenched with water (20 mL). To the mixture was then added di-tert-butyl dicarbonate (1.19 g, 5.46 mmol), and the mixture was stirred at ambient temperature for 2 h. The mixture was concentrated in vacuo and the residue was purified by preparative thin layer chromatography, eluting with 50% of ethyl acetate in petroleum ether, to provide a colorless solid (0.265 g). To it was added a 1 M solution hydrogen chloride in ethyl acetate (5 mL), and the mixture was stirred at ambient temperature for 2 h. To it was added potassium carbonate (0.010 g), and the mixture was filtered. Concentration of the filtrate in vacuo provided the title compound as a yellowish oil (0.075 g, 28% yield).

### Step 2. Preparation of N-(6-chloropyridin-3-yl)-6-((methyl(1H-pyrazol-4-yl)amino)methyl)isoquinolin-1-amine

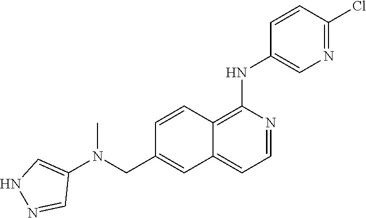 [SMILES: CN(Cc1ccc2c(Nc3ccc(Cl)nc3)nccc2c1)c1cn[nH]c1]

Following the procedure as described for EXAMPLE 248, Step 2, and making variations as required to replace 1H-pyrazol-4-amine with N-methyl-1H-pyrazol-4-amine, the title compound was obtained as a colorless solid (0.035 g, 18% yield): ¹H NMR (400 MHz, CDCl₃) δ 12.53-11.86 (m, 1H), 9.44 (s, 1H), 8.89 (d, J=2.4 Hz, 1H), 8.49-8.40 (m, 2H), 8.01 (d, J=6.0 Hz, 1H), 7.76 (s, 1H), 7.60 (d, J=8.8 Hz, 1H), 7.46 (d, J=8.8 Hz, 1H), 7.26-7.20 (m, 3H), 4.28 (s, 2H), 2.65 (s, 3H); MS (ES+) m/z 365.1 (M+1), 367.1 (M+1).

### Example 251

### Synthesis of N⁸-(6-chloropyridin-3-yl)-N³-((1-methyl-1H-pyrazol-4-yl)methyl)-1,7-naphthyridine-3,8-diamine

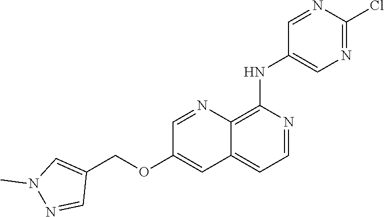 [SMILES: Cn1cc(COc2cnc3c(Nc4cnc(Cl)nc4)nccc3c2)cn1]

### Step 1. Preparation of 5-fluoro-3-methylpicolinonitrile

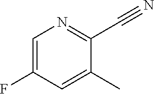

To a solution of 2-bromo-5-fluoro-3-methylpyridine (18.0 g, 94.7 mmol) in N,N-dimethylformamide (108 mL) was added zinc cyanide (6.67 g, 56.8 mmol) and [1,1′-bis(diphenylphosphino)ferrocene]dichloropalladium(II), complex with dichloromethane (4.64 g, 5.68 mmol). The reaction mixture was heated to 120° C. for 16 h. After cooling to ambient temperature, the mixture was poured into ice water (300 mL) and extracted with ethyl acetate (3×100 mL). The combined organic layers were washed with brine (100 mL) and concentrated in vacuo. The obtained residue was purified by silica gel column chromatography, eluting with a gradient of 0 to 100% of ethyl acetate in petroleum ether, to provide the title compound as a greenish solid (8.10 g, 63% yield): ¹H NMR (400 MHz, CDCl₃) δ 8.34 (s, 1H), 7.37-7.32 (m, 1H), 2.53 (s, 3H); MS (ES+) m/z 137 (M+1).

### Step 2. Preparation of 5-fluoro-3-methylpicolinamide

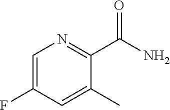

A solution of 5-fluoro-3-methylpicolinonitrile (1.00 g, 7.35 mmol) in concentrated sulfuric acid (5 mL) was heated to 80° C. for 30 minutes. After cooling to ambient temperature, the mixture was poured into ice water (50.0 mL), followed by addition of saturated sodium carbonate solution (50 mL) until pH 7 was reached. The resulting mixture was extracted with dichloromethane (3×100 mL). The combined organic phase was dried over anhydrous sodium sulfate, filtered, and the filtrate was concentrated in vacuo to provide the title compound as an off-white solid (1.10 g, 97% yield): ¹H NMR (400 MHz, DMSO-d₆) δ 8.41 (d, J=2.4 Hz, 1H), 7.91 (br s, 1H), 7.71 (dd, J=9.6, 2.4 Hz, 1H), 7.49 (br s, 1H), 2.55 (s, 3H); MS (ES+) m/z 155 (M+1).

### Step 3. Preparation of (E)-N-((dimethylamino)methylene)-5-fluoro-3-methylpicolinamide

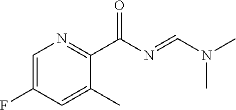

To a solution of 5-fluoro-3-methylpicolinamide (0.550 g, 3.57 mmol) in tetrahydrofuran (5 mL) was added N,N-dimethylformamide dimethyl acetal (1.28 g, 10.7 mmol, 1.42 mL) and the reaction mixture was heated to 80° C. for 18 h. The reaction was repeated on the same scale (0.55 g of 5-fluoro-3-methylpicolinamide). After cooling to ambient temperature, the mixtures were combined, filtered, and concentrated in vacuo to provide the title compound as a brown solid (0.70 g, 94% yield).

### Step 4. Preparation of 3-fluoro-1,7-naphthyridin-8(7H)-one

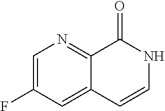 [SMILES: O=c1[nH]ccc2cc(F)cnc12]

To a solution of (E)-N-((dimethylamino)methylene)-5-fluoro-3-methylpicolinamide (0.700 g, 3.35 mmol) in tetrahydrofuran (1 mL) was added potassium tert-butoxide (0.751 g, 6.69 mmol) and the reaction mixture was heated to 80° C. for 12 h. After cooling to ambient temperature, the solid was filtered off to give the title compound as a yellow solid (0.495 g, 90% yield): ¹H NMR (400 MHz, DMSO-d₆) δ8.31 (d, J=2.8 Hz, 1H), 7.61 (d, J=5.5 Hz, 1H), 7.51 (dd, J=10.3, 2.8 Hz, 1H), 6.10 (d, J=5.6 Hz, 1H), NH not observed.

### Step 5. Preparation of 8-chloro-3-fluoro-1,7-naphthyridine

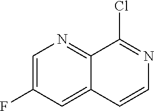 [SMILES: Fc1cnc2c(Cl)nccc2c1]

To a solution of 3-fluoro-1,7-naphthyridin-8(7H)-one (0.940 g, 5.73 mmol) in toluene (6 mL) was added N,N-diisopropylethylamine (2.22 g, 17.2 mmol) and phosphorus(V) oxychloride (2.63 g, 17.2 mmol). The reaction mixture was stirred at 130° C. for 16 h. After cooling to ambient temperature, the mixture was poured into ice water (10 mL) and was extracted with ethyl acetate (3×3 mL). The combined organic phase was washed with brine (3 mL), dried over anhydrous sodium sulfate, and filtered. The filtrate was concentrated in vacuo to afford the title compound as a brown solid (0.630 g, 60% yield): ¹H NMR (400 MHz, DMSO-d₆) δ9.19 (d, J=2.8 Hz, 1H), 8.47-8.42 (m, 2H), 7.98 (d, J=5.6 Hz, 1H); MS (ES+) m/z 183.0 (M+1), 185.0 (M+1).

### Step 6. Preparation of N-(6-chloropyridin-3-yl)-3-fluoro-1,7-naphthyridin-8-amine

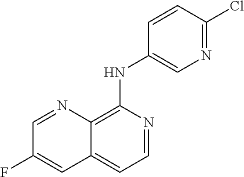 [SMILES: Fc1cnc2c(Nc3ccc(Cl)nc3)nccc2c1]

To a mixture of 8-chloro-3-fluoro-1,7-naphthyridine (0.200 g, 1.10 mmol), 6-chloropyridin-3-amine (0.127 g, 0.986 mmol), and potassium phosphate tribasic (0.465 g, 2.19 mmol) in toluene (3 mL) was added 2-dicyclohexylphosphino-2′,6′-dimethoxybiphenyl (0.090 g, 0.219 mmol) and tris(dibenzylideneacetone)dipalladium(0) (0.050 g, 0.055 mmol). The reaction mixture was degassed by purging with nitrogen, and then stirred at 100° C. for 6 h. After cooling to ambient temperature, the mixture was poured into ice water (3 mL) and extracted with ethyl acetate (3×1 mL). The combined organic phase was washed with brine (1 mL) and concentrated in vacuo. The residue purified by preparative thin-layer chromatography, eluting with 33% of ethyl acetate in petroleum ether, to afford the title compound as a yellow solid (0.140 g, 47% yield): ¹H NMR (400 MHz, CDCl₃) δ9.05-9.00 (m, 1H), 8.76-8.66 (m, 3H), 8.17 (d, J=5.6 Hz, 1H), 7.70 (dd, J=8.4, 2.4 Hz, 1H), 7.35 (d, J=8.4 Hz, 1H), 7.08 (d, J=6.0 Hz, 1H).

### Step 7. Preparation of N⁸-(6-chloropyridin-3-yl)-N³-((1-methyl-1H-pyrazol-4-yl)methyl)-1,7-naphthyridine-3,8-diamine

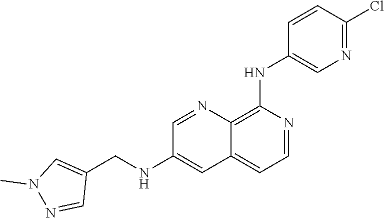 [SMILES: Cn1cc(CNc2cnc3c(Nc4ccc(Cl)nc4)nccc3c2)cn1]

To a solution of N-(6-chloropyridin-3-yl)-3-fluoro-1,7-naphthyridin-8-amine (0.156 g, 0.568 mmol) and (1-methylpyrazol-4-yl)methanamine (0.189 g, 1.70 mmol) in dimethyl sulfoxide (2 mL) was added potassium carbonate (0.157 g, 1.14 mmol). The reaction mixture was stirred at 140° C. for 16 h. After cooling to ambient temperature, the mixture was filtered. Purification of the filtrate by reverse-phase preparative HPLC (Waters XBridge BEH C18 100×30 mm, 10 μm column), eluting with a gradient of 35 to 65% of acetonitrile in water (containing 10 mM of ammonium carbonate), provided the title compound was a colorless solid (0.032 g, 15% yield): ¹H NMR (400 MHz, DMSO-d₆) δ 9.66 (s, 1H), 9.06 (d, J=2.8 Hz, 1H), 8.65 (dd, J=8.8, 2.8 Hz, 1H), 8.50 (d, J=2.8 Hz, 1H), 7.90 (d, J=5.6 Hz, 1H), 7.71 (s, 1H), 7.46 (s, 1H), 7.42 (d, J=8.8 Hz, 1H), 7.05-6.98 (m, 3H), 4.21 (d, J=5.2 Hz, 2H), 3.80 (s, 3H); MS (ES+) m/z 366.1 (M+1), 368.1 (M+1).

### Example 252

### Synthesis of N-(6-chloropyridin-3-yl)-3-((1-methyl-1H-pyrazol-4-yl)methoxy)-1,7-naphthyridin-8-amine

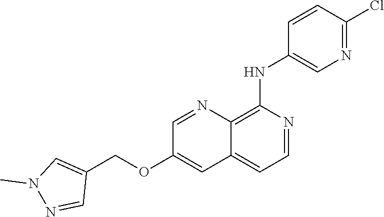 [SMILES: Cn1cc(COc2cnc3c(Nc4ccc(Cl)nc4)nccc3c2)cn1]

Following the procedure as described for EXAMPLE 251, and making variations as required to replace (1-methylpyrazol-4-yl)methanamine with (1-methyl-1H-pyrazol-4-yl)methanol, the title compound was obtained as a yellow solid (0.093 g, 26% yield): ¹H NMR (400 MHz, DMSO-d₆) δ 9.91 (s, 1H), 9.10 (d, J=2.8 Hz, 1H), 8.65 (dd, J=8.8, 2.8 Hz, 1H), 8.62 (d, J=2.4 Hz, 1H), 8.08 (d, J=5.6 Hz, 1H), 7.90 (s, 1H), 7.88 (d, J=2.8 Hz, 1H), 7.60 (s, 1H), 7.46 (d, J=8.8 Hz, 1H), 7.22 (d, J=5.6 Hz, 1H), 5.18 (s, 2H), 3.84 (s, 3H); MS (ES+) m/z 367.1 (M+1), 369.0 (M+1).

### Example 253

### Synthesis of N-(2-chloropyrimidin-5-yl)-3-((1-methyl-1H-pyrazol-4-yl)methoxy)-1,7-naphthyridin-8-amine

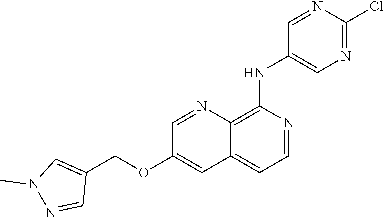 [SMILES: Cn1cc(COc2cnc3c(Nc4cnc(Cl)nc4)nccc3c2)cn1]

### Step 1. Preparation of N-(2-chloropyrimidin-5-yl)-3-fluoro-1,7-naphthyridin-8-amine

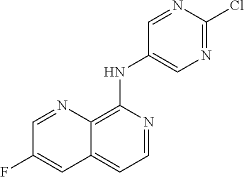 [SMILES: Fc1cnc2c(Nc3cnc(Cl)nc3)nccc2c1]

To a solution of 8-chloro-3-fluoro-1,7-naphthyridine (0.850 g, 4.66 mmol) and 2-chloropyrimidin-5-amine (0.724 g, 5.59 mmol) in toluene (5 mL) was added was added potassium phosphate tribasic (1.98 g, 9.31 mmol), 2-dicyclohexylphosphino-2′,6′-dimethoxybiphenyl (0.382 g, 0.931 mmol) and tris(dibenzylideneacetone)dipalladium(0) (0.213 g, 0.233 mmol). The reaction mixture was degassed by purging with nitrogen, and then stirred at 100° C. for 6 h. After cooling to ambient temperature, the mixture was poured into ice water (5 mL) and extracted with ethyl acetate (3×2 mL). The combined organic phase was washed with brine (2 mL) and concentrated in vacuo. The residue was triturated with acetonitrile (5 mL) to provide the title compound as a brown solid (0.880 g, 69% yield).

### Step 2. Preparation of N-(2-chloropyrimidin-5-yl)-3-((1-methyl-1H-pyrazol-4-yl)methoxy)-1,7-naphthyridin-8-amine

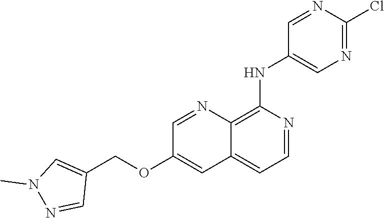 [SMILES: Cn1cc(COc2cnc3c(Nc4cnc(Cl)nc4)nccc3c2)cn1]

To a mixture of N-(2-chloropyrimidin-5-yl)-3-fluoro-1,7-naphthyridin-8-amine (0.100 g, 0.363 mmol) and (1-methyl-1H-pyrazol-4-yl)methanol (0.041 g, 0.363 mmol) in dimethyl sulfoxide (2 mL) was added potassium carbonate (0.100 g, 0.726 mmol). The reaction mixture was stirred at 100° C. for 36 h. After cooling to ambient temperature, the mixture was filtered and the filtrate was concentrated in vacuo. Purification of the residue by reverse-phase preparative HPLC (Waters XBridge BEH C18 250×50 mm×10 μm column), eluting with a gradient of 30 to 55% of acetonitrile in water (containing 10 mM of ammonium carbonate), followed by trituration in tert-butyl methyl ether (3 mL), provided the title compound was a yellow solid (0.100 g, 75% yield): ¹H NMR (400 MHz, DMSO-d₆) δ 10.18 (s, 1H), 9.51 (s, 2H), 8.65 (d, J=2.8 Hz, 1H), 8.11 (d, J=5.6 Hz, 1H), 7.91 (s, 2H), 7.61 (s, 1H), 7.28 (d, J=5.6 Hz, 1H), 5.18 (s, 2H), 3.84 (s, 3H); MS (ES+) m/z 368.0 (M+1), 370.0 (M+1).

### Example 254

### Synthesis of 1-(((8-((2-chloropyrimidin-5-yl)amino)-1,7-naphthyridin-3-yl)oxy)methyl)cyclopropane-1-carbonitrile

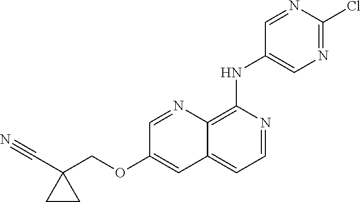 [SMILES: N#CC1(COc2cnc3c(Nc4cnc(Cl)nc4)nccc3c2)CC1]

To a solution of 1-(hydroxymethyl)cyclopropane-1-carbonitrile (0.232 g, 2.39 mmol) in THF (5 mL) was added NaH (60% dispersion in mineral oil, 0.958 g, 2.39 mmol) at 0° C. and the mixture was stirred at 0° C. for 30 minutes. To it was then added N-(2-chloropyrimidin-5-yl)-3-fluoro-1,7-naphthyridin-8-amine (0.330 g, 1.20 mmol). The mixture was stirred at 0° C. for 30 minutes, allowed to warm to ambient temperature, and stirred for 16 h. The mixture was poured into ice water (20 mL) and extracted with ethyl acetate (3×10 mL). The combined organic phase was washed with brine (10 mL) and concentrated in vacuo to afford the title compound as a colorless solid (0.025 g, 6% yield): ¹H NMR (400 MHz, DMSO-d₆) δ 10.24 (s, 1H), 9.52 (s, 2H), 8.76 (d, J=2.8 Hz, 1H), 8.12 (d, J=5.6 Hz, 1H), 7.75 (d, J=2.8 Hz, 1H), 7.24 (d, J=5.6 Hz, 1H), 4.30 (s, 2H), 1.43-1.49 (m, 2H), 1.23-1.28 (m, 2H); MS (ES+) m/z 353.0 (M+1), 355.0 (M+1).

### Example 255

### Synthesis of N-(6-chloropyridin-3-yl)-3-(cyclopropylmethoxy)-1,7-naphthyridin-8-amine

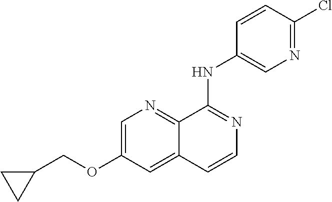 [SMILES: Clc1ccc(Nc2nccc3cc(OCC4CC4)cnc23)cn1]

### Step 1. Preparation of 3-chloro-5-methoxypicolinonitrile

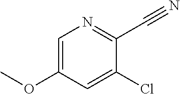

To a solution of 3,5-dichloropicolinonitrile (50.0 g, 289 mmol) in N,N-dimethylformamide (150 mL) was added sodium methoxide (15.6 g, 289 mmol) portion wise at 0° C. The mixture was stirred at 0° C. for 5 minutes, allowed to warm to ambient temperature, and stirred for 30 minutes. The mixture was then poured into water (200 mL) and extracted with ethyl acetate (4×150 mL). The combined organic extracts were dried over anhydrous sodium sulfate, filtered, and the filtrate was concentrated in vacuo. The obtained residue was purified by silica gel column chromatography, eluting with 33% of ethyl acetate in petroleum ether, to give the title compound as a light yellow solid (18.0 g, 37% yield): ¹H NMR (400 MHz, CDCl₃) δ8.30-8.27 (m, 1H), 7.31 (d, J=2.4 Hz, 1H), 3.96 (s, 3H).

### Step 2. Preparation of 5-methoxy-3-((triethylsilyl)ethynyl)picolinonitrile

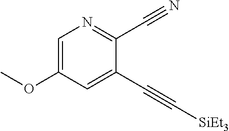

To a mixture of 3-chloro-5-methoxypicolinonitrile (6.00 g, 35.6 mmol), 2-triethylsilylacetylene (5.99 g, 42.7 mmol) and cesium carbonate (29.0 g, 89.0 mmol) in in tetrahydrofuran (100 mL) was added 2-dicyclohexylphosphino-2,4,6-triisopropylbiphenyl (3.39 g, 7.12 mmol) and palladium(II) acetate (0.799 g, 3.56 mmol) and the mixture was stirred at 60° C. for 12 h. After cooling to ambient temperature, the mixture was concentrated under reduced pressure. The obtained residue was purified by silica gel column chromatography, eluting with a gradient of 3-9% of ethyl acetate in petroleum ether, to give the title compound as a yellow solid (4.30 g, 44% yield): ¹H NMR (400 MHz, CDCl₃) δ8.21 (d, J=2.8 Hz, 1H), 7.19 (d, J=2.8 Hz, 1H), 3.85 (s, 3H), 1.01 (t, J=7.8 Hz, 9H), 0.66 (q, J=8.0 Hz, 6H).

### Step 3. Preparation of 3-(2,2-dimethoxyethyl)-5-methoxypicolinonitrile

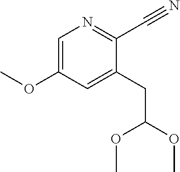

To a mixture of 5-methoxy-3-((triethylsilyl)ethynyl)picolinonitrile (5.00 g, 18.4 mmol) in methanol (50 mL) was added a 5 M solution of sodium methoxide in methanol (9.2 mL, 46.0 mmol) and the reaction mixture was stirred at 60° C. for 12 h. After cooling to ambient temperature, the reaction mixture was concentrated under reduced pressure. The obtained residue was diluted with water (100) mL and extracted with ethyl acetate (3×200 mL). The combined organic layer was washed with brine (3×200 mL), dried over anhydrous sodium sulfate, and filtered. Concentration of the filtrate under reduced pressure afforded the title compound as a yellow solid (3.70 g, 74% yield): ¹H NMR (400 MHz, CDCl₃) δ8.18 (d, J=2.8 Hz, 1H), 7.14 (d, J=2.8 Hz, 1H), 4.50 (t, J=5.2 Hz, 1H), 3.84 (s, 3H), 3.33 (s, 6H), 3.05 (d, J=5.1 Hz, 2H).

### Step 4. Preparation of 3-(2,2-dimethoxyethyl)-5-methoxypicolinamide

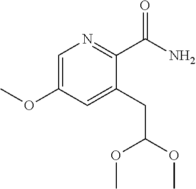

To a mixture of 3-(2,2-dimethoxyethyl)-5-methoxypicolinonitrile (3.70 g, 16.7 mmol) and 3 M aqueous sodium carbonate (67 mL, 201 mmol) in water (200 mL) and acetone (50 mL) was added 35% hydrogen peroxide (48 mL, 583 mmol) and the mixture was stirred at ambient temperature for 12 h. The reaction mixture was then quenched by addition of saturated sodium sulfite solution (500 mL) and extracted with dichloromethane (3×300 mL). The combined organic layers were washed with brine (300 mL), dried over anhydrous sodium sulfate, and filtered. Concentration of the filtrate under reduced pressure afforded the title compound as a yellow solid (3.80 g, 95% yield) as a yellow solid: ¹H NMR (400 MHz, CDCl₃) δ 8.12 (d, J=2.8 Hz, 1H), 7.82 (br s, 1H), 7.14 (d, J=2.8 Hz, 1H), 5.54 (br s, 1H), 4.65 (t, J=5.4 Hz, 1H), 3.90 (s, 3H), 3.44 (d, J=5.4 Hz, 2H), 3.38 (s, 6H).

### Step 5. Preparation of 3-methoxy-1,7-naphthyridin-8(7H)-one

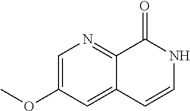

To a mixture of 3-(2,2-dimethoxyethyl)-5-methoxypicolinamide (3.80 g, 15.8 mmol) in toluene (50 mL) was added p-toluenesulfonic acid (0.272 g, 1.58 mmol) and the mixture was stirred at 120° C. for 12 h. After cooling to ambient temperature, the mixture was concentrated under reduced pressure. The obtained residue was triturated with ethyl acetate (5 mL) for 1 h. The mixture was filtered and the filter cake was collected and dried under reduced pressure. The solid residue was dissolved in a mixture of dichloromethane (100 mL) and methanol (100 mL) and then filtered through a pad of aluminium hydroxide. The filtrate was concentrated under reduced pressure to give the title compound as a grey solid (1.70 g, 61% yield): ¹H NMR (400 MHz, DMSO-d₆) δ 11.35 (br s, 1H), 8.42 (d, J=2.8 Hz, 1H), 7.55 (d, J=2.8 Hz, 1H), 7.23 (d, J=1.6 Hz, 1H), 6.48 (d, J=7.0 Hz, 1H), 3.92 (s, 3H).

### Step 6. Preparation of 8-chloro-3-methoxy-1,7-naphthyridine

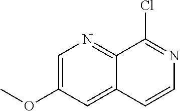

To a solution of 3-methoxy-1,7-naphthyridin-8(7H)-one (1.70 g, 9.65 mmol) in acetonitrile (40 mL) was added phosphorus oxychloride (7.30 g, 47.6 mmol) and the reaction mixture was heated to 85° C. for 12 h. After cooling to ambient temperature, the reaction mixture was poured into saturated sodium bicarbonate (100 mL). The mixture was stirred at ambient temperature for 30 minutes and was then extracted with dichloromethane (3×100 mL). The combined organic phase was washed with brine (200 mL), dried over anhydrous sodium sulfate, and filtered. Concentration of the filtrate under reduced pressure afforded the title compound as a yellow solid (1.50 g, 80% yield): ¹H NMR (400 MHz, DMSO-d₆) δ8.83 (d, J=2.8 Hz, 1H), 8.31 (d, J=5.6 Hz, 1H), 7.89 (d, J=2.8 Hz, 1H), 7.84 (d, J=5.6 Hz, 1H), 3.99 (s, 3H).

### Step 7. Preparation of 8-chloro-1,7-naphthyridin-3-ol

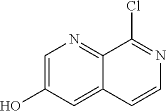 [SMILES: Oc1cnc2c(Cl)nccc2c1]

To a mixture of 8-chloro-3-methoxy-1,7-naphthyridine (1.00 g, 5.14 mmol) and tetrabutylammonium iodide (2.47 g, 6.68 mmol) in dichloromethane (20 mL) was added a solution of boron tribromide (12.9 g, 51.4 mmol) in dichloromethane (20 mL) and the reaction mixture was stirred at ambient temperature for 12 h. The mixture was then cooled to 0° C. and carefully quenched by dropwise addition of water (10 mL) until bubbling ceased. The mixture was allowed to warm to ambient temperature and stirred for 1 h, before dichloromethane (50 mL) and water (50 mL) were added to it. The mixture then was neutralized by addition of solid sodium bicarbonate. The layers were separated, and the aqueous layer was extracted with dichloromethane (3×50 mL). The combined organic layers were washed with brine (10 mL), dried over anhydrous sodium sulfate, and filtered. Concentration of the filtrate under reduced pressure and purification of the residue by reversed phase column chromatography, eluting with acetonitrile in water (containing 0.1% of formic acid), afforded the title compound as a yellow solid (0.500 g, 54% yield): ¹H NMR (400 MHz, DMSO-d₆) δ11.32 (s, 1H), 8.75 (d, J=2.8 Hz, 1H), 8.22 (d, J=5.6 Hz, 1H), 7.78 (d, J=5.6 Hz, 1H), 7.58 (d, J=2.8 Hz, 1H).

### Step 8. Preparation of 8-chloro-3-(cyclopropylmethoxy)-1,7-naphthyridine

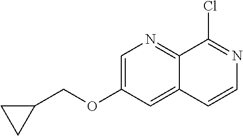 [SMILES: Clc1nccc2cc(OCC3CC3)cnc12]

To a mixture of 8-chloro-3-methoxy-1,7-naphthyridine (0.500 g, 2.77 mmol) and potassium carbonate (0.765 g, 5.54 mmol) in N,N-dimethylformamide (2 mL) was slowly added (bromomethyl)cyclopropane (0.748 g, 5.54 mmol) and the reaction mixture was heated to 90° C. for 2 h. After cooling to ambient temperature, the reaction mixture was poured into water (20 mL). The mixture was extracted with ethyl acetate (3×50 mL). The combined organic layers were washed with brine (50 mL), dried over anhydrous sodium sulfate, and filtered. Concentration of the filtrate under reduced pressure and purification of the residue by silica gel column chromatography, eluting with 50% of ethyl acetate in petroleum ether, afforded the title compound as a colorless solid (0.500 g, 77% yield): ¹H NMR (400 MHz, DMSO-d₆) δ8.86 (d, J=2.8 Hz, 1H), 8.30 (d, J=5.6 Hz, 1H), 7.86 (d, J=2.8 Hz, 1H), 7.81 (d, J=5.6 Hz, 1H), 4.07 (d, J=7.2 Hz, 2H), 1.43-1.28 (m, 1H), 0.69-0.59 (m, 2H), 0.45-0.37 (m, 2H).

### Step 9. Preparation of N-(6-chloropyridin-3-yl)-3-(cyclopropylmethoxy)-1,7-naphthyridin-8-amine

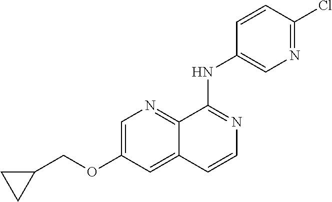 [SMILES: Clc1ccc(Nc2nccc3cc(OCC4CC4)cnc23)cn1]

To a mixture of 8-chloro-3-(cyclopropylmethoxy)-1,7-naphthyridine(0.100 g, 0.426 mmol), 6-chloropyridin-3-amine (0.055 g, 0.426 mmol), and cesium carbonate (0.417 g, 1.28 mmol) in 2-methyl-2-butanol (5 mL) was added methanesulfonato(2-di-t— butylphosphino-2,4,6-tri-i-propyl-1,1-biphenyl)(2-amino-1,1-biphenyl-2-yl)palladium(II) (0.034 g, 0.043 mmol) and the reaction mixture was stirred at 90° C. for 12 h. After cooling to ambient temperature, water (10 mL) was added, and the mixture was extracted with ethyl acetate (3×20 mL). The combined organic layer was washed with brine (3×10 mL), dried over anhydrous sodium sulfate, and filtered. The filtrate was concentrated in vacuo and the obtained residue purified by silica gel column chromatography, eluting with a gradient of 0-50% of ethyl acetate in petroleum, followed by trituration with methanol (3 mL), to afford the title compound as a colorless solid (0.030 g, 20% yield): ¹H NMR (400 MHz, DMSO-d₆) δ 9.92 ((, 1H), 9.11 (d, J=2.8 Hz, 1H), 8.72-8.57 (m, 2H), 8.06 (d, J=5.6 Hz, 1H), 7.70 (d, J=2.8 Hz, 1H), 7.45 (d, J=8.8 Hz, 1H), 7.18 (d, J=5.6 Hz, 12H), 4.10-4.00 (m, 2H), 1.38-1.27 (m, 1H), 0.67-0.57 (, 2H), 0.46-0.31 (, 2H); MS (ES+) m/z 327.1 (M+1), 329.1 (M+1)

### Examples 256-259

In a similar manner as described in EXAMPLE 255, utilizing the appropriately substituted starting materials and intermediates, the following compounds were prepared:

| | | MS | |
|---|---|---|---|
| Compound | | (ES+) | |
| No. | Name | m/z | NMR |
| 256 | 3-(cyclopropylmethoxy)- | 307.1 | ¹H NMR (400 MHZ, DMSO-d₆) δ |
| | N-(6-methylpyridin- | (M + 1). | 9.58 (s, 1H), 9.04 (d, J = 2.5 Hz, |
| | 3-yl)-1,7-naphthyridin- | | 1H), 8.64 (t, J = 2.5 Hz, 1H), 8.42 |
| | 8-amine | | (dd, J = 8.4, 2.7 Hz, 1H), 8.02 |
| | | | (dd, J = 5.7, 1.0 Hz, 1H), 7.67 (d, |
| | | | J = 2.7 Hz, 1H), 7.20 (d, J = 8.4 |
| | | | Hz, 1H), 7.11 (dd, J = 5.8, 2.1 Hz, |
| | | | 1H), 4.04 (dd, J = 7.1, 2.6 Hz, |
| | | | 2H), 2.43 (s, 3H), 1.37-1.30 (m, |
| | | | 1H), 0.67-0.62 (m, 2H), 0.43-0.39 |
| | | | (m, 2H). |
| 257 | 3-(cyclopropylmethoxy)- | 308.1 | ¹H NMR (400 MHZ, DMSO-d₆) δ |
| | N-(2-methylpyrimidin- | (M + 1). | 9.84 (s, 1H), 9.37 (s, 2H), 8.67 (d, |
| | 5-yl)-1,7-naphthyridin- | | J = 2.8 Hz, 1H), 8.05 (d, J = 5.7 |
| | 8-amine | | Hz, 1H), 7.71 (d, J = 2.8 Hz, 1H), |
| | | | 7.17 (d, J = 5.9 Hz, 1H), 4.06 (d, J = |
| | | | 7.1 Hz, 2H), 2.58 (s, 3H), 1.39- |
| | | | 1.29 (m, 1H), 0.67-0.60 (m, 2H), |
| | | | 0.46-0.37 (m, 2H). |
| 258 | 3-((1-methyl-1H-pyrazol- | 348.2 | ¹H NMR (400 MHZ, DMSO-d₆) δ |
| | 4-yl)methoxy)-N-(2- | (M + 1). | 9.84 (s, 1H), 9.36 (s, 2H), 8.63 (d, |
| | methylpyrimidin-5- | | J = 2.8 Hz, 1H), 8.07 (d, J = 5.7 |
| | yl)-1,7-naphthyridin- | | Hz, 1H), 7.91 (s, 1H), 7.88 (d, J = |
| | 8-amine | | 2.8 Hz, 1H), 7.61 (d, J = 0.7 Hz, |
| | | | 1H), 7.21 (d, J = 5.8 Hz, 1H), 5.19 |
| | | | (s, 2H), 3.85 (s, 3H), 2.58 (s, 3H). |
| 259 | N-(6-chloropyridin-3- | 357.0 | ¹H NMR (400 MHZ, DMSO-d₆) δ |
| | yl)-3-((3-methyloxetan- | (M + 1), | 9.95 (s, 1H), 9.11 (d, J = 2.8 Hz, |
| | 3-yl)methoxy)-1,7- | 359.0 | 1H), 8.72-8.61 (m, 2H), 8.07 (d, J = |
| | naphthyridin-8-amine | (M + 1). | 5.6 Hz, 1H), 7.81 (d, J = 2.8 Hz, |
| | | | 1H), 7.45 (d, J = 8.8 Hz, 1H), 7.20 |
| | | | (d, J = 6.0 Hz, 1H), 4.56 (d, J = |
| | | | 5.6 Hz, 2H), 4.36 (d, J = 5.6 Hz, |
| | | | 2H), 4.28 (s, 2H), 1.43 (s, 3H). |

### Example 260

### Synthesis of 6-(3-(1H-pyrazol-4-yl)propoxy)-N-(6-chloropyridin-3-yl)isoquinolin-1-amine

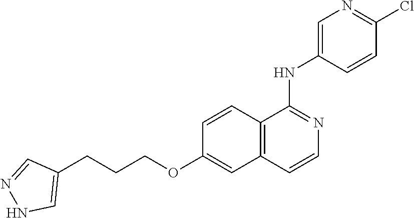 [SMILES: Clc1ccc(Nc2nccc3cc(OCCCc4cn[nH]c4)ccc23)cn1]

A mixture of 1-((6-chloropyridin-3-yl)amino)isoquinolin-6-ol (0.070 g, 0.258 mmol), 3-(1H-pyrazol-4-yl)propan-1-ol (0.039 g, 0.309 mmol), and triphenylphosphine (0.081 g, 0.309 mmol) in tetrahydrofuran (0.6 mL) was stirred for 10 minutes at the ambient temperature. The mixture was cooled to 0° C., and diethyl azodicarboxylate (0.049 mL, 0.309 mmol) was slowly added to it. The reaction mixture was allowed to warm up to ambient temperature and stirred for 2 h. The mixture was diluted with saturated sodium bicarbonate solution (20 mL) and extracted with ethyl acetate (3×20 mL). The combined organic layers were dried over anhydrous sodium sulfate and filtered. The filtrate was concentrated in vacuo and the obtained residue was purified by silica gel column chromatography, eluting with a gradient of 0 to 100% of ethyl acetate in heptane, to provide the title compound as a colorless solid (0.0278 g, 28% yield): ¹H NMR (400 MHz, DMSO-d₆) δ 12.59 (br s, 1H), 9.38 (s, 1H), 8.88 (d, J=2.8 Hz, 1H), 8.45-8.41 (m, 2H), 7.96 (d, J=5.7 Hz, 1H), 7.46-7.44 (m, 3H), 7.30-7.27 (m, 2H), 7.18 (d, J=5.8 Hz, 1H), 4.14 (t, J=6.4 Hz, 2H), 2.64 (t, J=7.5 Hz, 2H), 2.09-2.01 (m, 2H); MS (ES+) m/z 380.2 (M+1), 382.2 (M+1).

### Example 261

### Synthesis of (R)-3-((1-((6-chloropyridin-3-yl)amino)isoquinolin-6-yl)oxy)tetrahydrothiophene 1,1-dioxide

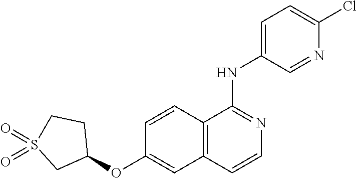 [SMILES: O=S1(=O)CC[C@@H](Oc2ccc3c(Nc4ccc(Cl)nc4)nccc3c2)C1]

### Step 1. Preparation of (R)—N-(6-chloropyridin-3-yl)-6-((tetrahydrothiophen-3-yl)oxy)isoquinolin-1-amine

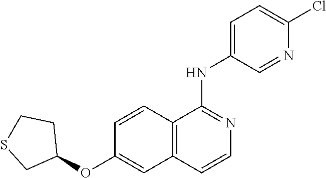 [SMILES: Clc1ccc(Nc2nccc3cc(O[C@@H]4CCSC4)ccc23)cn1]

To a solution of (3R)-thiolan-3-ol (0.014 g, 0.136 mmol) in N,N-dimethylformamide (2 mL) was added a 1.0 M solution of potassium tert-butoxide (0.16 mL, 0.16 mmol) in tetrahydrofuran, and the resulting mixture was stirred at ambient temperature for 5 minutes. To the reaction mixture was then added a solution of N-(6-chloropyridin-3-yl)-6-fluoro-N-((2-(trimethylsilyl)ethoxy)methyl)isoquinolin-1-amine (0.055 g, 0.136 mmol). The reaction mixture was stirred at ambient temperature for 1 h, and then concentrated in vacuo. The obtained residue was dissolved in dichloromethane (2 mL), and to it was added trifluoroacetic acid (1 mL, 13.0 mmol). The resulting mixture was stirred at ambient temperature for 2 h, and then concentrated in vacuo to give a residue. The residue was diluted with ethyl acetate (30 mL), and the mixture was washed with saturated sodium bicarbonate solution (20 mL) and brine (20 mL), dried over anhydrous sodium sulfate, and filtered. The filtrate was concentrated in vacuo to provide a residue, which was purified by silica gel column chromatography, eluting with a gradient of 10 to 100% of ethyl acetate in heptane, to afford the title compound as a colorless solid (0.053 g, quantitative yield): ¹H NMR (400 MHz, CDCl₃) δ8.53-8.52 (m, 1H), 8.38-8.35 (m, 1H), 8.04 (d, J=6.0 Hz, 1H), 7.88 (d, J=9.3 Hz, 1H), 7.34 (d, J=8.6 Hz, 1H), 7.24-7.21 (m, 1H), 7.14-7.11 (m, 2H), 5.32-5.29 (m, 1H), 3.28 (dd, J=11.9, 4.7 Hz, 1H), 3.19-3.12 (m, 2H), 3.05-3.00 (m, 1H), 2.58-2.52 (m, 1H), 2.20-2.11 (m, 1H), NH not observed; MS (ES+) m/z 358.2 (M+1), 360.2 (M+1).

### Step 2. Preparation of (R)-3-((1-((6-chloropyridin-3-yl)amino)isoquinolin-6-yl)oxy)tetrahydrothiophene 1,1-dioxide

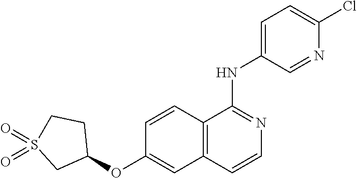 [SMILES: O=S1(=O)CC[C@@H](Oc2ccc3c(Nc4ccc(Cl)nc4)nccc3c2)C1]

To a mixture of (R)—N-(0-chloropyridin-3-yl)-6-((tetrahydrothiophen-3-yl)oxy)isoquinolin-1-amine (0.040 g, 0.112 mmol) in dichloromethane (2 mL) and acetonitrile (1 mL) was added 3-chloroperoxybenzoic acid (0.063 g, 0.279 mmol) at 0° C. and the reaction mixture was stirred at that temperature for 1 h. The reaction mixture was then diluted with ethyl acetate (30 mL) and washed with saturated sodium bicarbonate (30 mL) and brine (30 mL). The organic phase was dried over anhydrous sodium sulfate and filtered. The filtrate was concentrated in vacuo to give the title compound as a colourless solid (0.014 g, 30% yield): ¹H NMR (400 MHz, DMSO-d₆) δ 9.45-9.42 (m, 1H), 8.89 (t, J=3.3 Hz, 1H), 8.51-8.48 (m, 1H), 8.44-8.41 (m, 1H), 8.00-7.98 (m, 1H), 7.49-7.45 (m, 1H), 7.39-7.35 (m, 1H), 7.33-7.30 (m, 1H), 7.20-7.19 (m, 1H), 5.49-5.45 (m, 1H), 3.72 (dd, J=14.4, 6.3 Hz, 1H), 3.32-3.29 (m, 2H), 3.14-3.00 (m, 1H), 2.68-2.57 (m, 1H), 2.48-2.45 (m, 1H); MS (ES+) m/z 390.2 (M+1), 392.2 (M+1).

### Example 262

### Synthesis of (S)-3-((1-((6-chloropyridin-3-yl)amino)isoquinolin-6-yl)oxy)tetrahydrothiophene 1,1-dioxide

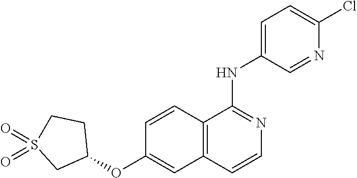 [SMILES: O=S1(=O)CC[C@H](Oc2ccc3c(Nc4ccc(Cl)nc4)nccc3c2)C1]

Following the procedure as described for EXAMPLE 261, and making variations as required to replace (3R)-thiolan-3-ol with (3S)-thiolan-3-ol, the title compound was obtained as a colorless solid (0.011 g, 24% yield): ¹H NMR (400 MHz, DMSO-d₆) δ9.46-9.44 (m, 1H), 8.88 (d, J=2.7 Hz, 1H), 8.49 (d, J=9.2 Hz, 1H), 8.44-8.41 (m, 1H), 7.98 (d, J=5.8 Hz, 1H), 7.46 (d, J=8.7 Hz, 1H), 7.37 (d, J=2.5 Hz, 1H), 7.32 (dd, J=9.1, 2.5 Hz, 1H), 7.20 (d, J=5.7 Hz, 1H), 5.49-5.45 (m, 1H), 3.73 (dd, J=14.4, 6.4 Hz, 1H), 3.32-3.29 (m, 2H), 3.10 (qd, J=7.3, 4.8 Hz, 1H), 2.68-2.57 (m, 1H), 2.48-2.44 (m, 1H); MS (ES+) m/z 390.2 (M+1), 392.2 (M+1).

### Example 263

### Synthesis of N-(6-chloropyridin-3-yl)-6-((tetrahydrofuran-3-yl)oxy)isoquinolin-1-amine

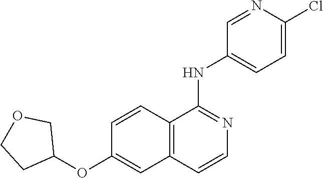 [SMILES: Clc1ccc(Nc2nccc3cc(OC4CCOC4)ccc23)cn1]

To a solution of 1-chloro-6-fluoroisoquinoline (0.100 g, 0.551 mmol) and tetrahydrofuran-3-ol (0.045 mL, 0.551 mmol) in N,N-dimethylformamide (2 mL) was added a 1.0 M solution of potassium tert-butoxide in tetrahydrofuran (0.55 mL, 0.551 mmol). The reaction mixture was stirred at ambient temperature for 15 minutes, and then diluted with 1,4-dioxane (4 mL). To the mixture was added 5-amino-2-chloropyridine (0.071 g, 0.551 mmol), tris(dibenzylideneacetone)dipalladium(0) (0.038 g, 0.041 mmol), 2-dicyclohexylphosphino-2′,6′-dimethoxy-1,1′-biphenyl (0.034 g, 0.083 mmol), and potassium phosphate tribasic (0.234 g, 1.10 mmol). The resulting mixture was degassed by passing a stream of nitrogen through it for 5 minutes and then heated to 110° C. for 5 h. After cooling to ambient temperature, the reaction mixture was filtered through celite, and the filtrate was concentrated in vacuo. The obtained residue was purified by silica gel chromatography, eluting with a gradient of 0 to 100% of ethyl acetate in heptane, to afford the title compound as a colorless solid (0.027 g, 14% yield): ¹H NMR (400 MHz, DMSO-d₆) δ 9.39 (s, 1H), 8.88 (d, J=2.7 Hz, 1H), 8.46-8.41 (m, 2H), 7.97 (d, J=5.8 Hz, 1H), 7.45 (d, J=8.7 Hz, 1H), 7.26 (dd, J=7.5, 2.4 Hz, 2H), 7.19 (d, J=5.8 Hz, 1H), 5.23-5.21 (m, 1H), 3.98 (dd, J=10.3, 4.5 Hz, 1H), 3.89 (dd, J=15.1, 8.3 Hz, 2H), 3.80 (td, J=8.4, 4.7 Hz, 1H), 2.38-2.29 (m, 1H), 2.08-2.01 (m, 1H); MS (ES+) m/z 342.5 (M+1), 344.5 (M+1).

### Examples 264 and 265

### Synthesis of trans-4-((1-((6-chloropyridin-3-yl)amino)isoquinolin-6-yl)oxy)-1-methylcyclohexan-1-ol and cis-4-((1-((6-chloropyridin-3-yl)amino)isoquinolin-6-yl)oxy)-1-methylcyclohexan-1-ol

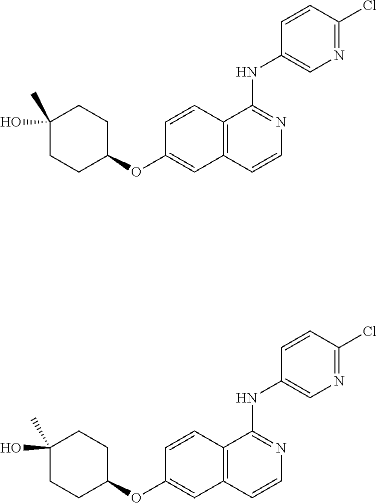 [SMILES: C[C@]1(O)CC[C@H](Oc2ccc3c(Nc4ccc(Cl)nc4)nccc3c2)CC1 ; C[C@]1(O)CC[C@@H](Oc2ccc3c(Nc4ccc(Cl)nc4)nccc3c2)CC1]

### Step 1. Preparation of 4-((1-((6-chloropyridin-3-yl)((2-(trimethylsilyl)ethoxy)methyl)amino)isoquinolin-6-yl)oxy)cyclohexan-1-one

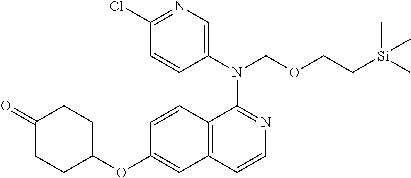 [SMILES: C[Si](C)(C)CCOCN(c1ccc(Cl)nc1)c1nccc2cc(OC3CCC(=O)CC3)ccc12]

To a solution of 4-hydroxycyclohexanone (0.060 g, 0.526 mmol) in N,N-dimethylformamide (2 mL) was added sodium hydride (60% dispersion in mineral oil, 0.022 g, 0.557 mmol). The reaction mixture was stirred at ambient temperature for 5 minutes, and then a solution of N-(6-chloropyridin-3-yl)-6-fluoro-N-((2-(trimethylsilyl)ethoxy)-methyl)isoquinolin-1-amine (0.150 g, 0.371 mmol) in N,N-dimethylformamide (3 mL) was added to it. The mixture was stirred at ambient temperature for 2 h. The mixture was diluted with ethyl acetate (40 mL) and washed with saturated aqueous sodium bicarbonate (40 mL) and brine (40 mL). The organic phase was dried over anhydrous sodium sulfate and filtered. The filtrate was concentrated in vacuo to provide the title product as a light-yellow oil (0.145 g, 78% yield): MS (ES+) m/z 498.8 (M+1), 500.8 (M+1).

### Step 2. Preparation of trans-4-((1-((6-chloropyridin-3-yl)amino)isoquinolin-6-yl)oxy)-1-methylcyclohexan-1-ol and cis-4-((1-((6-chloropyridin-3-yl)amino)isoquinolin-6-yl)oxy)-1-methylcyclohexan-1-ol

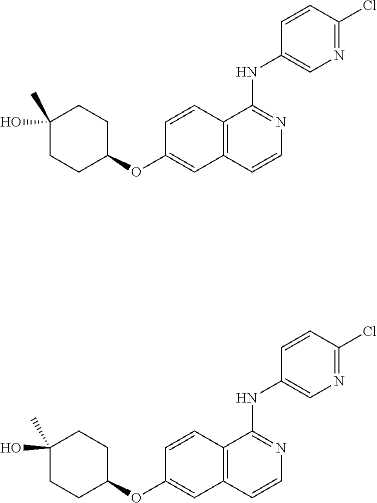 [SMILES: C[C@]1(O)CC[C@H](Oc2ccc3c(Nc4ccc(Cl)nc4)nccc3c2)CC1 ; C[C@]1(O)CC[C@@H](Oc2ccc3c(Nc4ccc(Cl)nc4)nccc3c2)CC1]

To a solution of 4-((1-((6-chloropyridin-3-yl)((2-(trimethylsilyl)ethoxy)methyl)amino)isoquinolin-6-yl)oxy)cyclohexan-1-one (0.145 g, 0.291 mmol) in tetrahydrofuran (2 mL) was added a sodium hydride (60% dispersion in mineral oil, 0.99 g, 24.8 mmol) at 0° C. The reaction mixture was allowed to warm to ambient temperature and stirred for 20 minutes. To the reaction mixture was then added a 3 M solution of methylmagnesium bromide in tetrahydrofuran (0.37 mL, 1.11 mmol). The mixture was stirred at ambient temperature for 2 h, and then concentrated in vacuo. The obtained residue was dissolved in dichloromethane (2 mL) and to it was added trifluoroacetic acid (1 mL). The mixture was stirred at ambient temperature for 2 h, and then concentrated in vacuo to give a residue. Purification and separation of the mixture by chiral SFC (Lux Cell-4, 10×250 mm, 5 μm column), eluting with 45% of methanol (containing 10 mM ammonium formate) in supercritical carbon dioxide, afforded the title compounds as single isomers as colorless solids. First eluting isomer (0.0026 g, 2% yield): ¹H NMR (400 MHz, CD₃CN) δ8.80 (d, J=2.8 Hz, 1H), 8.35 (dd, J=8.7, 2.9 Hz, 1H), 8.17-8.15 (m, 1H), 7.96 (d, J=5.8 Hz, 2H), 7.36 (dd, J=8.7, 0.4 Hz, 1H), 7.26-7.23 (m, 2H), 7.15 (d, J=5.9 Hz, 1H), 4.74-4.70 (m, 1H), 2.44 (br s, 1H), 2.08-2.00 (m, 2H), 1.84-1.72 (m, 4H), 1.55-1.49 (m, 2H), 1.23 (s, 3H); MS (ES+) m/z 384.2 (M+1), 386.2 (M+1). Second eluting isomer (0.0033 g, 2% yield): ¹H NMR (400 MHz, CD₃CN) δ8.80 (d, J=2.8 Hz, 1H), 8.35 (dd, J=8.7, 2.8 Hz, 1H), 8.17-8.14 (m, 1H), 7.96 (d, J=5.8 Hz, 2H), 7.36 (dd, J=8.7, 0.4 Hz, 1H), 7.24-7.21 (m, 2H), 7.15 (d, J=5.7 Hz, 1H), 4.53-4.46 (m, 1H), 1.95-1.93 (m, 2H), 1.90-1.80 (m, 2H), 1.76-1.70 (m, 2H), 1.61-1.53 (m, 2H), 1.23 (s, 3H), OH not observed; MS (ES+) m/z 384.2 (M+1), 386.2 (M+1).

### Example 266

### Synthesis of N-(6-chloropyridin-3-yl)-6-(pyridin-3-yloxy)isoquinolin-1-amine

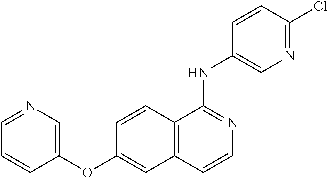 [SMILES: Clc1ccc(Nc2nccc3cc(Oc4cccnc4)ccc23)cn1]

To a solution of 3-hydroxypyridine (0.014 g, 0.149 mmol) and N-(6-chloropyridin-3-yl)-6-fluoro-N-((2-(trimethylsilyl)ethoxy)methyl)isoquinolin-1-amine (0.050 g, 0.124 mmol) in N,N-dimethylformamide (2 mL) was added sodium carbonate (0.051 g, 0.371 mmol). The resulting mixture was stirred at 100° C. for 4 h. After cooling to ambient temperature, the reaction mixture was filtered, and the filtrate was concentrated in vacuo. The obtained residue was dissolved in dichloromethane (2 mL), and to it was added trifluoroacetic acid (1 mL, 7.4 mmol) at ambient temperature. The mixture was stirred at ambient temperature for 2 h, and then concentrated in vacuo to provide a residue. Purification of the residue by reverse-phase preparative HPLC, eluting with a gradient of 15 to 60% of acetonitrile in water (containing 0.5% of formic acid), provided the title compound as a colorless solid (0.0044 g, 10% yield): ¹H NMR (400 MHz, CD₃CN) δ 8.82 (d, J=2.8 Hz, 1H), 8.52-8.48 (m, 2H), 8.37-8.34 (m, 1H), 8.31 (d, J=9.2 Hz, 1H), 8.08-8.06 (m, 1H), 8.01 (d, J=5.8 Hz, 1H), 7.56 (ddd, J=8.4, 2.8, 1.4 Hz, 1H), 7.48-7.45 (m, 1H), 7.42 (dd, J=9.1, 2.5 Hz, 1H), 7.38 (d, J=8.7 Hz, 1H), 7.27 (d, J=2.5 Hz, 1H), 7.14 (d, J=5.7 Hz, 1H); MS (ES+) m/z 349.2 (M+1), 351.2 (M+1).

### Example 267

### Synthesis of 6-((1H-pyrazol-4-yl)oxy)-N-(6-chloropyridin-3-yl)isoquinolin-1-amine

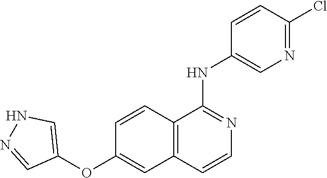 [SMILES: Clc1ccc(Nc2nccc3cc(Oc4cn[nH]c4)ccc23)cn1]

Following the procedure as described for EXAMPLE 266, and making variations as required to replace 3-hydroxypyridine with 1H-pyrazol-4-ol, the title compound was obtained as a colorless solid (0.011 g, 8% yield): ¹H NMR (400 MHz, DMSO-d₆) δ 8.81-8.80 (m, 1H), 8.60-8.57 (m, 1H), 8.33-8.28 (m, 1H), 7.86-7.75 (m, 2H), 7.61-7.50 (m, 3H), 7.33-7.25 (m, 2H); MS (ES+) m/z 338.0 (M+1), 340.0 (M+1).

### Example 268

### Synthesis of ((1-((6-chloropyridin-3-yl)amino)isoquinolin-6-yl)imino)(cyclopropyl)(methyl)-λ⁶-sulfanone

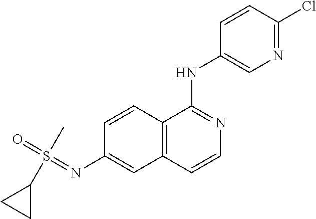 [SMILES: CS(=O)(=Nc1ccc2c(Nc3ccc(Cl)nc3)nccc2c1)C1CC1]

### Step 1. Preparation of (methylsulfinyl)cyclopropane

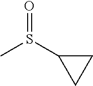

To a solution of bromocyclopropane (2.00 g, 16.5 mmol) in dimethyl sulfoxide (20 mL) was added sodium thiomethoxide (3.04 g, 43.3 mmol) and potassium tert-butoxide (2.23 g, 19.9 mmol) and the mixture was stirred at 100° C. for 12 h. After cooling to ambient temperature, the reaction mixture was diluted with water (20 mL) and extracted with ethyl acetate (40 mL). The organic layer was washed with brine (20 mL), dried over anhydrous sodium sulfate, and filtered. To the filtrate was added methanol (20 mL), water (20 mL), and sodium periodate (3.54 g, 16.5 mmol) and the resulting mixture was stirred at ambient temperature for 12 h. The reaction mixture was diluted with water (100 mL) and extracted with ethyl acetate (3×100 mL). The combined organic extracts were washed with brine (100 mL), dried over anhydrous sodium sulfate, and filtered. The filtrate was concentrated under reduced pressure and the obtained residue was purified by silica gel column chromatography, eluting with a gradient of 0 to 20% of methanol in ethyl acetate to give the title compound as yellow oil (0.900 g, 52% yield): ¹H NMR (400 MHz, CDCl₃) δ2.64 (d, J=1.6 Hz, 3H), 2.20-2.12 (m, 1H), 1.07-1.05 (m, 1H), 0.99-0.96 (m, 2H), 0.87-0.80 (m, 1H).

### Step 2. Preparation of benzyl (cyclopropyl(methyl)(oxo)-λ⁶-sulfaneylidene)carbamate

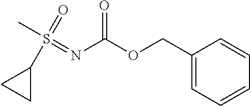 [SMILES: CS(=O)(=NC(=O)OCc1ccccc1)C1CC1]

A mixture of (methylsulfinyl)cyclopropane (0.200 g, 1.92 mmol), benzyl carbamate (0.435 g, 2.88 mmol), rhodium(II) acetate dimer (0.0765 g, 0.173 mmol), iodobenzene diacetate (0.928 g, 2.88 mmol), and magnesium oxide (0.310 g, 7.68 mmol) in dichloromethane (3 mL) was stirred at ambient temperature for 24 h. The reaction mixture was filtered through a bed of celite, and the filtrate was concentrated in vacuo. The obtained residue was purified by reverse phase preparative HPLC (Phenomenex Luna C18 150×25 mm, 10 μm column), eluting with a gradient of 22 to 52% of acetonitrile in water (containing 0.1% of formic acid) to yield the title compound as a colorless solid (0.090 g, 17% yield): ¹H NMR (400 MHz, DMSO-d₆) δ7.40-7.30 (m, 5H), 5.00 (s, 2H), 3.39 (s, 3H), 2.99-2.90 (m, 1H), 1.15-1.06 (m, 4H).

### Step 3. Preparation of cyclopropyl(imino)(methyl)-λ⁶-sulfanone

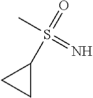

To a mixture of benzyl (cyclopropyl(methyl)(oxo)-λ⁶-sulfaneylidene)carbamate (0.440 g, 1.74 mmol) in methanol (20 mL) was added 10% palladium on carbon (0.739 g). The mixture was purged with hydrogen three times and then stirred at 50° C. for 16 h under a hydrogen atmosphere of 15 psi. After cooling to ambient temperature, the reaction mixture was filtered through a pad of celite, and the filter cake was washed with methanol (30 mL). The combined filtrate was concentrated under reduced pressure to give the title compound was as colorless oil (0.300 g, quantitative yield): ¹H NMR (400 MHz, CDCl₃) δ3.04 (s, 3H), 2.70-2.55 (m, 1H), 2.38 (br s, 1H), 1.95-1.21-1.14 (m, 2H), 1.09-1.02 (m, 2H).

### Step 4. Preparation of 6-bromo-N-(6-chloropyridin-3-yl)isoquinolin-1-amine

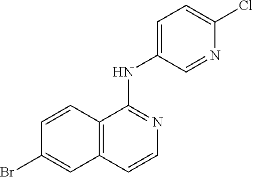 [SMILES: Clc1ccc(Nc2nccc3cc(Br)ccc23)cn1]

To a solution of 6-bromo-1-chloroisoquinoline (10.1 g, 41.6 mmol) in propan-2-ol (100 mL) was added a 4 M solution of hydrogen chloride in dioxane (15.6 mL, 62.4 mmol) and 6-chloropyridin-3-amine (5.88 g, 45.7 mmol) and the reaction mixture was stirred at 70° C. for 18 h. After cooling to ambient temperature, saturated sodium bicarbonate solution (600 mL) was added to it and the mixture was extracted with ethyl acetate (3×600 mL). The combined organic phase was washed with brine (400 mL), dried over anhydrous sodium sulfate, and filtered. The filtrate was concentrated in vacuo and the obtained residue was triturated with ethyl acetate (400 mL) at ambient temperature for 12 h to give the title compound as an off-white solid (11.5 g, 83% yield): ¹H NMR (400 MHz, DMSO-d₆) δ9.55 (s, 1H), 8.88 (d, J=2.8 Hz, 1H), 8.51-8.44 (m, 1H), 8.41 (dd, J=8.8, 2.8 Hz, 1H), 8.16 (d, J=1.6 Hz, 1H), 8.06 (d, J=5.6 Hz, 1H), 7.82 (dd, J=8.8, 1.6 Hz, 1H), 7.46 (d, J=8.8 Hz, 1H), 7.28-7.22 (m, 1H).

### Step 5. Preparation of ((1-((6-chloropyridin-3-yl)amino)isoquinolin-6-yl)imino)(cyclopropyl)(methyl)-λ⁶-sulfanone

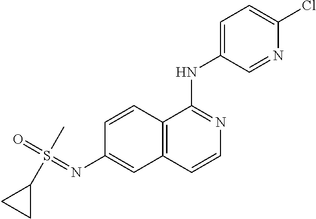 [SMILES: CS(=O)(=Nc1ccc2c(Nc3ccc(Cl)nc3)nccc2c1)C1CC1]

To a mixture of cyclopropyl(imino)(methyl)-λ⁶-sulfanone (0.220 g, 1.85 mmol) and 6-bromo-N-(6-chloropyridin-3-yl)isoquinolin-1-amine (0.617 g, 1.85 mmol) in dioxane (22 mL) was added tris(dibenzylideneacetone)dipalladium(0) (0.169 g, 0.185 mmol), sodium tert-butoxide (0.355 g, 3.69 mmol) and 4,5-bis(diphenylphosphino)-9,9-dimethylxanthene (0.214 g, 0.369 mmol). The reaction mixture was stirred at 100° C. for 16 h. After cooling to ambient temperature, the mixture was passed through a bed of silica gel, and the filtrate was concentrated in vacuo. The obtained residue was purified by silica gel column chromatography, eluting with a gradient of 0 to 100% of ethyl acetate in petroleum ether gradient, and then by reverse phase preparative HPLC (Phenomenex Luna C18 150×25 mm, 5 μm column), eluting with a gradient of 28 to 58% of acetonitrile in water (containing 0.1% of ammonium hydroxide) to yield the title compound as a colorless solid (0.240 g, 35% yield): ¹H NMR (400 MHz, DMSO-d₆) δ9.29 (s, 1H), 8.87 (d, J=2.8 Hz, 1H), 8.42 (dd, J=8.8, 2.8 Hz, 1H), 8.32 (d, J=9.2 Hz, 1H), 7.89 (d, J=5.6 Hz, 1H), 7.44 (d, J=8.8 Hz, 1H), 7.31-7.16 (m, 2H), 7.09 (d, J=5.6 Hz, 1H), 3.31 (s, 3H), 2.94-2.86 (m, 1H), 1.35-1.23 (m, 1H), 1.18-0.99 (m, 3H); MS (ES+) m/z 373.1 (M+1), 375.1 (M+1).

### Examples 269-272

In a similar manner as described in EXAMPLE 268, utilizing the appropriately substituted starting materials and intermediates, the following compounds were prepared:

| | | MS | |
|---|---|---|---|
| Compound | | (ES+) | |
| No. | Name | m/z | NMR |
| 269 | ((1-((6-chloropyridin-3- | 387.2 | ¹H NMR (400 MHZ, DMSO-d₆) δ |
| | yl)amino)isoquinolin- | (M + 1), | 9.28 (s, 1H), 8.86 (d, J = 2.8 Hz, |
| | 6-yl)imino)(cyclopropyl | 389.2 | 1H), 8.42 (dd, J = 8.8, 2.8 Hz, |
| | methyl)(methyl)-λ⁶-sulfanone | (M + 1). | 1H), 8.32 (d, J = 8.8 Hz, 1H), 7.88 |
| | | | (d, J = 5.6 Hz, 1H), 7.43 (d, J = |
| | | | 8.8 Hz, 1H), 7.28 (d, J = 2.4 Hz, |
| | | | 1H), 7.21 (dd, J = 8.8, 2.4 Hz, |
| | | | 1H), 7.08 (d, J = 6.0 Hz, 1H), |
| | | | 3.48-3.42 (m, 2H), 3.28 (s, 3H), |
| | | | 1.18-1.06 (m, 1H), 0.66-0.58 (m, |
| | | | 2H), 0.45-0.31 (m, 2H). |
| 270 | ((1-((6-chloropyridin-3- | 417.0 | ¹H NMR (400 MHZ, DMSO-d₆) δ |
| | yl)amino)isoquinolin-6- | (M + 1), | 9.29 (s, 1H), 8.86 (d, J = 2.8 Hz, |
| | yl)imino)(methyl)(tetrahydro- | 419.0 | 1H), 8.42 (dd, J = 8.8, 2.8 Hz, |
| | 2H-pyran-4-yl)-λ⁶-sulfanone | (M + 1). | 1H), 8.32 (d, J = 9.2 Hz, 1H), 7.89 |
| | | | (d, J = 5.6 Hz, 1H), 7.43 (d, J = |
| | | | 8.8 Hz, 1H), 7.30 (d, J = 2.4 Hz, |
| | | | 1H), 7.24 (dd, J = 8.8, 2.0 Hz, |
| | | | 1H), 7.08 (d, J = 5.6 Hz, 1H), |
| | | | 4.09-3.91 (m, 2H), 3.80-3.62 (m, |
| | | | 1H), 3.42-3.36 (m, 2H), 3.20 (s, |
| | | | 3H), 2.11-1.99 (m, 2H), 1.89- |
| | | | 1.74 (m, 2H). |
| 271 | ((1-((6-chloropyridin-3- | 417.1 | ¹H NMR (400 MHZ, CD₃OD) δ |
| | yl)amino)isoquinolin-6- | (M + 1), | 8.70 (d, J = 2.2 Hz, 1H), 8.24- |
| | yl)imino)(cyclopropyl)(2- | 419.1 | 8.18 (m, 2H), 7.82 (d, J = 6.4 Hz, |
| | methoxyethyl)-λ⁶-sulfanone | (M + 1). | 1H), 7.42-7.33 (m, 3H), 7.08 (d, J = |
| | | | 6.0 Hz, 1H), 3.91-3.84 (m, 2H), |
| | | | 3.68-3.64 (m, 2H), 3.37 (s, 3H), |
| | | | 2.89-2.79 (m, 1H), 1.39-1.34 (m, |
| | | | 1H), 1.23 (s, 3H). |
| 272 | ((1-((6-chloropyridin-3- | 403.1 | ¹H NMR (400 MHZ, DMSO-d₆) δ |
| | yl)amino)isoquinolin-6- | (M + 1), | 9.30 (s, 1H), 8.87 (d, J = 2.8 Hz, |
| | yl)imino)(methyl)(oxetan-3- | 405.1 | 1H), 8.42 (dd, J = 8.8, 2.8 Hz, |
| | ylmethyl)-λ⁶-sulfanone | (M + 1). | 1H), 8.32 (d, J = 8.8 Hz, 1H), 7.89 |
| | | | (d, J = 5.6 Hz, 1H), 7.43 (d, J = |
| | | | 8.8 Hz, 1H), 7.25 (d, J = 2.0 Hz, |
| | | | 1H), 7.17 (dd, J = 8.8, 2.0 Hz, |
| | | | 1H), 7.09 (d, J = 6.0 Hz, 1H), 4.66 |
| | | | (dd, J = 8.0, 6.0 Hz, 2H), 4.52 (t, J = |
| | | | 6.4 Hz, 1H), 4.43 (t, J = 6.4 Hz, |
| | | | 1H), 3.90 (d, J = 7.2 Hz, 2H), |
| | | | 3.67-3.63 (m, 1H), 3.25 (s, 3H). |

### Examples 273 and 274

### Synthesis of (R)-((1-((6-chloropyridin-3-yl)amino)isoquinolin-6-yl)imino)(cyclopropyl)(methyl)-λ⁶-sulfanone and (S)-((1-((6-chloropyridin-3-yl)amino)isoquinolin-6-yl)imino)(cyclopropyl)(methyl)-λ⁶-sulfanone

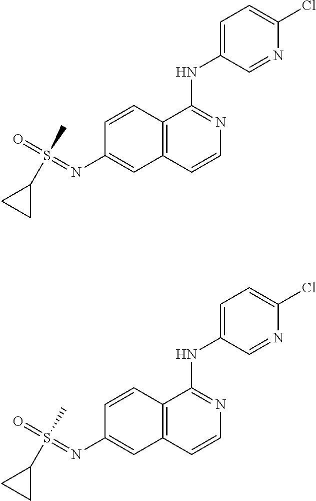 [SMILES: C[S@@](=O)(=Nc1ccc2c(Nc3ccc(Cl)nc3)nccc2c1)C1CC1 ; C[S@](=O)(=Nc1ccc2c(Nc3ccc(Cl)nc3)nccc2c1)C1CC1]

Racemic ((1-((6-chloropyridin-3-yl)amino)isoquinolin-6-yl)imino)(cyclopropyl)(methyl)-λ⁶-sulfanone was synthesized as described in EXAMPLE 268. Resolution of the enantiomers by chiral SFC (ChiralPak 00 250×30 mm, 10 μm column), eluting with 70% of a mixture of methanol and acetonitrile (containing 0.1% of ammonium hydroxide) in supercritical carbon dioxide, afforded the title compounds as single enantiomers as colorless solids. First eluting enantiomer (0.092 g, 36% yield): ¹H NMR (400 MHz, DMSO-d₆) δ9.28 (s, 1H), 8.87 (d, J=2.8 Hz, 1H), 8.42 (dd, J=8.8, 2.8 Hz, 1H), 8.31 (d, J=8.8 Hz, 1H), 7.89 (d, J=5.6 Hz, 1H), 7.43 (d, J=8.8 Hz, 1H), 7.30-7.20 (m, 2H), 7.08 (d, J=6.0 Hz, 1H), 3.30 (s, 3H), 3.00-2.81 (m, 1H), 1.39-1.20 (m, 1H), 1.19-0.96 (m, 3H); MS (ES+) m/z 373.0 (M+1), 375.0 (M+1). Second eluting enantiomer (0.112 g, 44% yield): ¹H NMR (400 MHz, DMSO-d₆) δ9.29 (s, 1H), 8.87 (d, J=2.8 Hz, 1H), 8.42 (dd, J=8.8, 2.8 Hz, 1H), 8.32 (d, J=9.2 Hz, 1H), 7.89 (d, J=5.6 Hz, 1H), 7.44 (d, J=8.8 Hz, 1H), 7.31-7.16 (m, 2H), 7.09 (d, J=6.0 Hz, 1H), 3.31 (s, 3H), 2.98-2.86 (m, 1H), 1.38-1.23 (m, 1H), 1.20-0.92 (m, 3H); MS (ES+) m/z 373.0 (M+1), 375.0 (M+1).

### Example 275

### Synthesis of ((1-((6-chloropyridin-3-yl)amino)isoquinolin-6-yl)imino)(methyl)(oxetan-3-yl)-λ⁶-sulfanone

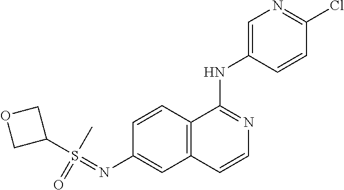 [SMILES: CS(=O)(=Nc1ccc2c(Nc3ccc(Cl)nc3)nccc2c1)C1COC1]

### Step 1. Preparation of 3-(methylsulfinyl)oxetane

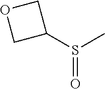

To a solution of 3-bromooxetane (2.00 g, 14.6 mmol) in acetonitrile (10 mL) was added sodium thiomethoxide (1.23 g, 17.5 mmol), and the mixture was heated in a microwave reactor at 100° C. for 1.5 h. After cooling to ambient temperature, methanol (29 mL) was added followed by a solution of sodium periodate (4.69 g, 21.9 mmol) in water (21 mL). The reaction mixture was stirred at ambient temperature for 24 h and then filtered through a pad of celite. The filter cake was washed with methanol (100 mL) and the combined filtrate was concentrated in vacuo to afford the title compound (1.70 g, 87% yield): ¹H NMR (500 MHz, CDCl₃) δ5.16-5.10 (m, 1H), 4.92 (t, J=7.7 Hz, 2H), 4.77 (t, J=7.7 Hz, 1H), 4.04-3.95 (m, 1H), 2.45 (s, 3H); MS (ES+) m/z 121.1 (M+1).

### Step 2. Preparation of benzyl (methyl(oxetan-3-yl)(oxo)-λ⁶-sulfaneylidene)carbamate

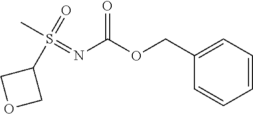 [SMILES: CS(=O)(=NC(=O)OCc1ccccc1)C1COC1]

To a mixture of 3-(methylsulfinyl)oxetane (1.70 g, 13.4 mmol), benzyl carbamate (3.21 g, 20.2 mmol), iodobenzene diacetate (6.83 g, 20.2 mmol), and magnesium oxide (2.17 g, 53.8 mmol) in dichloromethane (56 mL) was added rhodium(II) acetate dimer (0.535 g, 1.21 mmol), and the mixture was stirred at ambient temperature for 24 h. The mixture was filtered through a pad of celite, and the filter cake was washed with dichloromethane (80 mL). The combined filtrate was concentrated in vacuo and the obtained residue was purified by silica gel column chromatography, eluting with a gradient of 0-100% of ethyl acetate in hexanes, followed by a gradient of 0-30% of methanol in dichloromethane. The obtained residue was further purified by silica gel column chromatography, eluting with a gradient of 0-100% of ethyl acetate in hexanes, to afford the title compound as a colorless solid (0.500 g, 13% yield): ¹H NMR (300 MHz, CDCl₃) δ 7.45-7.26 (m, 5H), 5.11 (s, 2H), 5.09-5.03 (m, 1H), 5.01-4.88 (m, 2H), 4.76-4.64 (m, 1H), 4.43-4.30 (m, 1H), 3.26 (s, 3H); MS (ES+) m/z 270.0 (M+1).

### Step 3. Preparation of imino(methyl)(oxetan-3-yl)-λ⁶-sulfanone

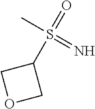

To a mixture of palladium (10% on carbon, 0.373 g) in methanol (5 mL) was added a solution of benzyl (methyl(oxetan-3-yl)(oxo)-A6-sulfaneylidene)carbamate (0.150 g, 0.501 mmol) in methanol (5 mL), and the mixture was stirred at ambient temperature under an atmosphere of 50 psi of hydrogen for 24 h. The mixture was then filtered through a pad of celite, and the filter cake was washed with methanol (10 mL). Concentration of the combined filtrate in vacuo afforded the title compound (0.0700 g, 83% yield): ¹H NMR (300 MHz, DMSO-d₆) δ4.85-4.58 (m, 5H), 4.58-4.46 (m, 1H), 2.97 (s, 3H).

### Step 4. Preparation of ((1-((6-chloropyridin-3-yl)amino)isoquinolin-6-yl)imino)(methyl)(oxetan-3-yl)-λ⁶-sulfanone

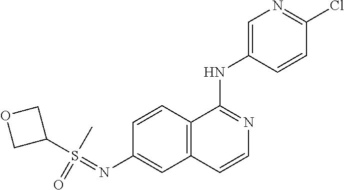 [SMILES: CS(=O)(=Nc1ccc2c(Nc3ccc(Cl)nc3)nccc2c1)C1COC1]

To a solution of 6-bromo-N-(6-chloropyridin-3-yl)isoquinolin-1-amine (0.100 g, 0.299 mmol), imino(methyl)(oxetan-3-yl)-λ⁶-sulfanone (0.056 g, 0.332 mmol), tris(dibenzylideneacetone)dipalladium(0) (0.027 g, 0.030 mmol), and 4,5-bis(diphenylphosphino)-9,9-dimethylxanthene (0.035 g, 0.060 mmol) in 1,4-dioxane (3 mL) was added sodium tert-butoxide (0.057 g, 0.597 mmol), and the mixture was stirred at 90° C. for 3 h. After cooling to ambient temperature, the mixture was passed through a bed of celite. The filter cake was washed with ethyl acetate (10 mL) and the combined filtrate was concentrated in vacuo. Purification of the residue by silica gel column chromatography, eluting with a gradient of 0-30% of methanol in dichloromethane, afforded the title compound as a colorless solid (0.042 g, 35% yield): ¹H NMR (300 MHz, DMSO-d₆) δ 9.29 (s, 1H), 8.86 (d, J=2.8 Hz, 1H), 8.40 (dd, J=8.7, 2.8 Hz, 1H), 8.33 (d, J=9.0 Hz, 1H), 7.89 (d, J=5.8 Hz, 1H), 7.42 (d, J=8.7 Hz, 1H), 7.30 (d, J=2.1 Hz, 1H), 7.22 (dd, J=9.0, 2.2 Hz, 1H), 7.09 (d, J=5.8 Hz, 1H), 5.02-4.83 (m, 5H), 3.28 (s, 3H); MS (ES+) m/z 389.1 (M+1), 391.1 (M+1).

### Examples 276 and 277

### Synthesis of (S)-((1-((6-chloropyridin-3-yl)amino)isoquinolin-6-yl)imino)(methyl)(oxetan-3-yl)-λ⁶-sulfanone) and (R)-((1-((6-chloropyridin-3-yl)amino)isoquinolin-6-yl)imino)(methyl)(oxetan-3-yl)-λ⁶-sulfanone)

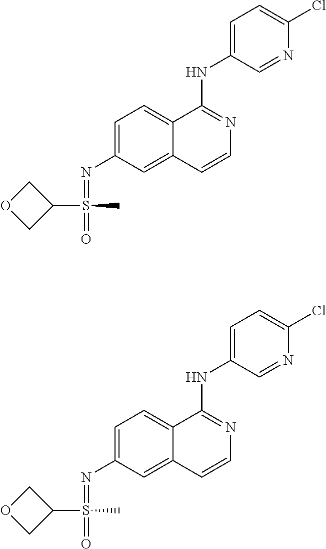 [SMILES: C[S@](=O)(=Nc1ccc2c(Nc3ccc(Cl)nc3)nccc2c1)C1COC1 ; C[S@@](=O)(=Nc1ccc2c(Nc3ccc(Cl)nc3)nccc2c1)C1COC1]

Racemic ((1-((6-chloropyridin-3-yl)amino)isoquinolin-6-yl)imino)(methyl)(oxetan-3-yl)-λ⁶-sulfanone was synthesized as described in EXAMPLE 275. Resolution of the enantiomers by chiral SFC (ChiralPak AS 250×30 mm, 10 μm column), eluting with 40% of a mixture of propan-2-ol and acetonitrile (containing 0.1% of ammonium hydroxide) in supercritical carbon dioxide, afforded the title compounds as single enantiomers as colorless solids. First eluting enantiomer (0.048 g, 34% yield): ¹H NMR (400 MHz, DMSO-d₆) δ9.31 (s, 1H), 8.87 (d, J=2.8 Hz, 1H), 8.42 (dd, J=8.8, 2.8 Hz, 1H), 8.34 (d, J=9.2 Hz, 1H), 7.91 (d, J=5.6 Hz, 1H), 7.43 (d, J=8.8 Hz, 1H), 7.32 (d, J=2.0 Hz, 1H), 7.24 (dd, J=8.8, 2.0 Hz, 1H), 7.11 (d, J=6.0 Hz, 1H), 5.01-4.96 (m, 1H), 4.94-4.85 (m, 4H), 3.30 (s, 3H); MS (ES+) m/z 389.1 (M+1), 391.1 (M+1). Second eluting enantiomer (0.058 g, 41% yield): ¹H NMR (400 MHz, DMSO-d₆) δ9.31 (s, 1H), 8.87 (d, J=2.8 Hz, 1H), 8.42 (dd, J=8.8, 2.8 Hz, 1H), 8.34 (d, J=9.2 Hz, 1H), 7.91 (d, J=6.0 Hz, 1H), 7.43 (d, J=8.8 Hz, 1H), 7.32 (d, J=2.0 Hz, 1H), 7.24 (dd, J=8.8, 2.0 Hz, 1H), 7.11 (d, J=6.0 Hz, 1H), 5.01-4.96 (m, 1H), 4.94-4.84 (m, 4H), 3.30 (s, 3H); MS (ES+) m/z 389.1 (M+1), 391.1 (M+1).

### Example 278

### Synthesis of cyclopropyl(methyl)((1-((2-methylpyrimidin-5-yl)amino)isoquinolin-6-yl)imino)-λ⁶-sulfanone

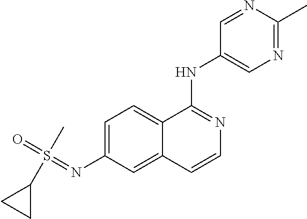 [SMILES: Cc1ncc(Nc2nccc3cc(N=S(C)(=O)C4CC4)ccc23)cn1]

### Step 1. Preparation of 6-bromo-N-(2-methylpyrimidin-5-yl)isoquinolin-1-amine

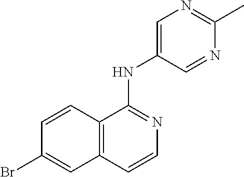 [SMILES: Cc1ncc(Nc2nccc3cc(Br)ccc23)cn1]

To a solution of 2-methylpyrimidin-5-amine (0.810 g, 7.42 mmol) in tetrahydrofuran (16.9 mL) was added lithium bis(trimethylsilyl)amide (1 M in tetrahydrofuran, 12.4 mL, 12.4 mmol) at −78° C. The reaction mixture was stirred for 5 minutes at that temperature, and then at 0° C. for 10 minutes. To it was then added 6-bromo-1-chloro-isoquinoline (1.20 g, 4.95 mmol), and the mixture was heated to 75° C. for 2 h. After cooling to ambient temperature, the mixture was diluted with water (40 mL), and the aqueous phase was extracted with ethyl acetate (3×80 mL). The combined organic phase was washed with brine (20 mL), dried over anhydrous sodium sulfate, and filtered. Concentration of the filtrate in vacuo and purification of the residue by silica gel column chromatography, eluting with a gradient of 0-100% of ethyl acetate in hexanes, afforded the title compound as a solid (1.20 g, 77% yield): ¹H NMR (400 MHz, DMSO-d₆) δ 9.48 (s, 1H), 9.15 (s, 2H), 8.46 (d, J=9.0 Hz, 1H), 8.16 (d, J=2.0 Hz, 1H), 8.05 (d, J=5.7 Hz, 1H), 7.82 (dd, J=9.0, 2.1 Hz, 1H), 7.23 (d, J=5.6 Hz, 1H), 2.58 (s, 3H); MS (ES+) m/z 316.1 (M+1).

### Step 2. Preparation of cyclopropyl(methyl)((1-((2-methylpyrimidin-5-yl)amino)isoquinolin-6-yl)imino)-λ⁶-sulfanone

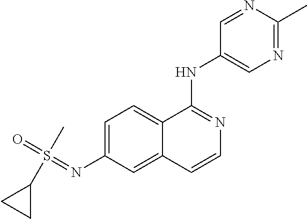 [SMILES: Cc1ncc(Nc2nccc3cc(N=S(C)(=O)C4CC4)ccc23)cn1]

To a solution of 6-bromo-N-(2-methylpyrimidin-5-yl)isoquinolin-1-amine (0.0670 g, 0.210 mmol), cyclopropyl(imino)(methyl)-λ⁶-sulfanone (0.0280 g, 0.230 mmol), tris(dibenzylideneacetone)dipalladium(0) (0.0200 g, 0.0210 mmol), and 4,5-bis(diphenylphosphino)-9,9-dimethylxanthene (0.0250 g, 0.0430 mmol) in 1,4-dioxane (2.10 mL) was added sodium tert-butoxide (0.041 g, 0.430 mmol), and the mixture was stirred at 90° C. for 3 h. After cooling to ambient temperature, the mixture was concentrated in vacuo. Purification of the residue by silica gel column chromatography, eluting with a gradient of 0-15% of methanol in dichloromethane, followed by reverse-phase preparative HPLC, eluting with a gradient of 19-29% of acetonitrile in water (containing 10 mM of ammonium formate), afforded the title compound as a solid (0.033 g, 44% yield): ¹H NMR (400 MHz, DMSO-d₆) δ9.19 (s, 1H), 9.15 (s, 2H), 8.30 (d, J=9.0 Hz, 1H), 7.87 (d, J=5.8 Hz, 1H), 7.25 (d, J=2.2 Hz, 1H), 7.22 (dd, J=8.9, 2.3 Hz, 1H), 7.06 (d, J=5.7 Hz, 1H), 3.30 (s, 3H), 2.93-2.86 (m, 1H), 2.57 (s, 3H), 1.31-1.22 (m, 1H), 1.17-1.02 (m, 3H); MS (ES+) m/z 354.2 (M+1).

### Example 279

### Synthesis of N-(6-chloropyridin-3-yl)-6-((1-(difluoromethyl)-1H-pyrazol-4-yl)methoxy)isoquinolin-1-amine

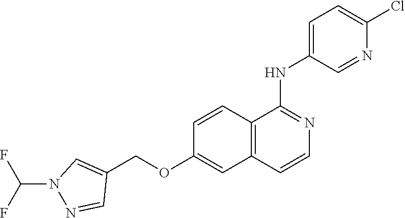 [SMILES: FC(F)n1cc(COc2ccc3c(Nc4ccc(Cl)nc4)nccc3c2)cn1]

### Step 1. Preparation of (1-(difluoromethyl)-1H-pyrazol-4-yl)methanol

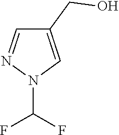

Following the procedure as described for EXAMPLE 151, Step 1, and making variations as required to replace 1-ethynylcyclopropane-1-carboxylic acid with 1-(difluoromethyl)-1H-pyrazole-4-carboxylic acid, the title compound was obtained as a yellow oil (0.0855 g, 62% yield): ¹H NMR (400 MHz, CDCl₃) δ 7.80 (s, 1H), 7.65 (s, 1H), 7.16 (t, J=60 Hz, 1H), 4.63 (s, 2H), OH not observed; ¹⁹F NMR (376 MHz, CDCl₃) δ−93.3 (s).

### Step 2. Preparation of N-(6-chloropyridin-3-yl)-6-((1-(difluoromethyl)-1H-pyrazol-4-yl)methoxy)isoquinolin-1-amine

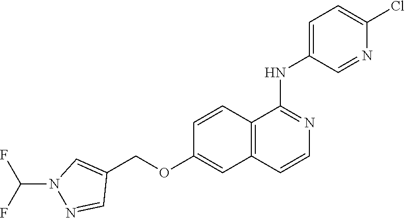 [SMILES: FC(F)n1cc(COc2ccc3c(Nc4ccc(Cl)nc4)nccc3c2)cn1]

To a solution of (1-(difluoromethyl)-1H-pyrazol-4-yl)methanol (0.0220 g, 0.149 mmol) in N,N-dimethylformamide (1 mL) was added N-(6-chloropyridin-3-yl)-6-fluoro-N-((2-(trimethylsilyl)ethoxy)methyl)isoquinolin-1-amine (0.0500 g, 0.124 mmol) and potassium tert-butoxide (1 M in tetrahydrofuran, 0.186 mL, 0.186 mmol) at ambient temperature, and the resulting mixture was heated to 60° C. for 2 h. After cooling to ambient temperature, the mixture was concentrated in vacuo. To the obtained residue was added dichloromethane (1 mL) and trifluoroacetic acid (0.190 mL, 2.48 mmol), and the reaction mixture was stirred at ambient temperature for 5 h. To the mixture was added saturated sodium bicarbonate solution (20 mL), and the mixture was extracted with dichloromethane (3×20 mL). The combined organic phase was washed with brine (20 mL), dried over anhydrous sodium sulfate, and filtered. The filtrate was concentrated in vacuo to provide a residue which was purified by silica gel column chromatography, eluting with a gradient of 0 to 60% of ethyl acetate in heptane, to afford the title compound as a colorless solid (0.0167 g, 33% yield): ¹H NMR (400 MHz, DMSO-d₆) δ9.39 (s, 1H), 8.88-8.88 (m, 1H), 8.46-8.41 (m, 3H), 7.99-7.97 (m, 2H), 7.84 (t, J=59.2 Hz, 1H), 7.46-7.43 (m, 2H), 7.33-7.30 (m, 1H), 7.21 (d, J=5.8 Hz, 1H), 5.19 (s, 2H); ¹⁹F NMR (376 MHz, DMSO-d₆) δ−94.0 (s); MS (ES+) m/z 402.2 (M+1), 404.2 (M+1).

### Examples 280-301

In a similar manner as described in EXAMPLE 279, utilizing the appropriately substituted starting materials and intermediates, the following compounds were prepared:

| | | MS | |
|---|---|---|---|
| Compound | | (ES+) | |
| No. | Name | m/z | NMR |
| 280 | (R)-6-((1- | 445.2 | ¹H NMR (400 MHZ, DMSO-d₆) δ 9.38 |
| | benzylpyrrolidin-2- | (M + 1), | (s, 1H), 8.88 (d, J = 2.7 Hz, 1H), 8.42 |
| | yl)methoxy)-N-(6- | 447.2 | (dd, J = 8.6, 2.7 Hz, 2H), 7.96 (d, J = |
| | chloropyridin-3- | (M + 1). | 5.8 Hz, 1H), 7.44 (d, J = 8.7 Hz, 1H), |
| | yl)isoquinolin-1-amine | | 7.33-7.26 (m, 7H), 7.18 (d, J = 6.0 Hz, |
| | | | 1H), 4.19-4.14 (m, 2H), 4.03-4.00 (m, |
| | | | 1H), 3.49-3.46 (m, 1H), 3.04-3.01 (m, |
| | | | 1H), 2.85-2.82 (m, 1H), 2.29-2.23 (m, |
| | | | 1H), 2.06-1.99 (m, 1H), 1.72-1.70 (m, 3H). |
| 281 | N-(6-chloropyridin-3- | 392.0 | ¹H NMR (400 MHZ, DMSO-d₆) δ 9.38 |
| | yl)-6-((5,6-dihydro-4H- | (M + 1), | (s, 1H), 8.88 (d, J = 2.6 Hz, 1H), 8.44- |
| | pyrrolo[1,2-b]pyrazol-3- | 394.0 | 8.41 (m, 2H), 7.96 (d, J = 5.7 Hz, 1H), |
| | yl)methoxy)isoquinolin- | (M + 1). | 7.57 (s, 1H), 7.45 (d, J = 8.7 Hz, 1H), |
| | 1-amine | | 7.38 (d, J = 2.0 Hz, 1H), 7.27 (dd, J = |
| | | | 9.2, 2.0 Hz, 1H), 7.19 (d, J = 5.8 Hz, |
| | | | 1H), 5.08 (s, 2H), 4.06 (t, J = 7.3 Hz, |
| | | | 2H), 2.88-2.85 (m, 2H), 2.56-2.53 (m, 2H). |
| 282 | 6-((1-benzylpyrrolidin- | 431.2 | ¹H NMR (400 MHZ, DMSO-d₆) δ 9.37 |
| | 3-yl)oxy)-N-(6- | (M + 1), | (d, J = 1.0 Hz, 1H), 8.88 (dd, J = 2.3 |
| | chloropyridin-3- | 433.2 | 0.4 Hz, 1H), 8.44-8.41 (m, 2H), 7.95 |
| | yl)isoquinolin-1-amine | (M + 1). | (d, J = 5.8 Hz, 1H), 7.45 (d, J = 8.7 |
| | | | Hz, 1H), 7.33 (d, J = 4.5 Hz, 4H), |
| | | | 7.26-7.17 (m, 4H), 5.06-5.04 (m, 1H), |
| | | | 3.63 (s, 2H), 2.95-2.90 (m, 1H), 2.75- |
| | | | 2.69 (m, 2H), 2.46-2.38 (m, 2H), 1.87- |
| | | | 1.82 (m, 1H). |
| 283 | (R)-6-((1- | 445.2 | ¹H NMR (400 MHZ, DMSO-d₆) δ 9.38 |
| | benzylpyrrolidin-3- | (M + 1), | (d, J = 0.2 Hz, 1H), 8.89-8.88 (m, 1H), |
| | yl)methoxy)-N-(6- | 447.2 | 8.43 (d, J = 9.0 Hz, 2H), 7.96 (dd, J = |
| | chloropyridin-3- | (M + 1). | 5.7, 0.2 Hz, 1H), 7.46-7.44 (m, 1H), |
| | yl)isoquinolin-1-amine | | 7.32-7.17 (m, 8H), 4.03 (d, J = 6.0 Hz, |
| | | | 2H), 3.60 (s, 2H), 2.66-2.64 (m, 3H), |
| | | | 2.41-2.39 (m, 2H), 2.02-1.99 (m, 1H), |
| | | | 1.58-1.55 (m, 1H). |
| 284 | (S)-6-((1- | 445.2 | ¹H NMR (400 MHZ, DMSO-d₆) δ 9.38 |
| | benzylpyrrolidin-3- | (M + 1), | (s, 1H), 8.88 (d, J = 2.7 Hz, 1H), 8.42 |
| | yl)methoxy)-N-(6- | 447.2 | (d, J = 9.0 Hz, 2H), 7.96 (d, J = 5.8 |
| | chloropyridin-3- | (M + 1). | Hz, 1H), 7.45 (d, J = 8.7 Hz, 1H), |
| | yl)isoquinolin-1-amine | | 7.32-7.24 (m, 7H), 7.18 (d, J = 5.7 Hz, |
| | | | 1H), 4.03 (d, J = 5.9 Hz, 2H), 3.59 (s, |
| | | | 2H), 2.66-2.58 (m, 3H), 2.42-2.39 (m, |
| | | | 2H), 2.03-1.99 (m, 1H), 1.59-1.55 (m, 1H). |
| 285 | N-(6-chloropyridin-3- | 406.2 | ¹H NMR (400 MHZ, DMSO-d₆) δ 9.39 |
| | yl)-6-((4,5,6, 7- | (M + 1), | (s, 1H), 8.88 (d, J = 2.3 Hz, 1H), 8.43 |
| | tetrahydropyrazolo[1,5- | 408.2 | (d, J = 9.0 Hz, 2H), 7.97 (d, J = 5.8 |
| | alpyridin-3- | (M + 1). | Hz, 1H), 7.54 (s, 1H), 7.45 (d, J = 8.7 |
| | yl)methoxy)isoquinolin- | | Hz, 1H), 7.40 (d, J = 1.9 Hz, 1H), 7.27 |
| | 1-amine | | (dd, J = 9.2, 2.1 Hz, 1H), 7.20 (d, J = |
| | | | 5.8 Hz, 1H), 5.06 (s, 2H), 4.06 (t, J = |
| | | | 6.0 Hz, 2H), 2.79 (t, J = 6.2 Hz, 2H), |
| | | | 1.98-1.94 (m, 2H), 1.82-1.79 (m, 2H). |
| 286 | N-(6-chloropyridin-3- | 404.2 | ¹H NMR (400 MHZ, DMSO-d₆) δ 8.82 |
| | yl)-6-((1-(methylsulfo- | (M + 1), | (s, 1H), 8.53 (d, J = 9.0 Hz, 1H), 8.33 |
| | nyl)cyclopropyl)me- | 406.2 | (d, J = 7.3 Hz, 1H), 7.88 (d, J = 4.8 |
| | thoxy)isoquinolin-1-amine | (M + 1). | Hz, 1H), 7.54 (d, J = 7.0 Hz, 1H), 7.44 |
| | | | (t, J = 7.1 Hz, 2H), 7.22 (d, J = 6.2 Hz, |
| | | | 1H), 4.51 (s, 2H), 3.19 (s, 3H), 1.49- |
| | | | 1.46 (m, 2H), 1.30-1.27 (m, 2H), NH |
| | | | not observed. |
| 287 | N-(6-chloropyridin-3- | 418.0 | ¹H NMR (400 MHZ, DMSO-d₆) δ 9.86 |
| | yl)-6-((1-(methylsulfo- | (M + 1), | (brs, 1H), 8.83 (d, J = 2.7 Hz, 1H), |
| | nyl)cyclobutyl)me- | 420.0 | 8.54 (s, 1H), 8.34 (d, J = 9.3 Hz, 1H), |
| | thoxy)isoquinolin- | (M + 1). | 7.90 (d, J = 6.5 Hz, 1H), 7.53 (d, J = |
| | 1-amine | | 9.0 Hz, 2H), 7.44 (d, J = 11.3 Hz, 1H), |
| | | | 7.24 (d, J = 6.1 Hz, 1H), 4.61 (s, 2H), |
| | | | 3.03 (s, 3H), 2.69-2.62 (m, 2H), 2.21- |
| | | | 2.17 (m, 3H), 2.02-1.98 (m, 1H). |
| 288 | N-(6-chloropyridin-3- | 370.2 | ¹H NMR (400 MHZ, CD₃OD) δ 8.62 |
| | yl)-6-((1-methoxycyclo- | (M + 1), | (d, J = 2.7 Hz, 1H), 8.46 (d, J = 10.1 |
| | butyl)methoxy)isoquinolin- | 372.2 | Hz, 1H), 8.05 (dd, J = 8.6, 2.8 Hz, |
| | 1-amine | (M + 1). | 1H), 7.64 (d, J = 8.5 Hz, 1H), 7.58 (d, |
| | | | J = 6.7 Hz, 1H), 7.49-7.47 (m, 2H), |
| | | | 7.34 (d, J = 6.7 Hz, 1H), 4.35 (s, 2H), |
| | | | 3.30 (s, 3H), 2.29 (dd, J = 10.1, 2.5 |
| | | | Hz, 2H), 2.19-2.13 (m, 2H), 1.89-1.86 |
| | | | (m, 1H), 1.79-1.74 (m, 1H), NH not |
| | | | observed. |
| 289 | N-(6-chloropyridin-3- | 392.2 | 1H NMR (400 MHZ, DMSO-d₆) δ 9.37 |
| | yl)-6-((1-cyclopropyl- | (M + 1), | (s, 1H), 8.87 (d, J = 2.8 Hz, 1H), 8.42 |
| | 1H-pyrazol-4- | 394.2 | (dd, J = 8.8, 2.9 Hz, 2H), 7.97 (d, J = |
| | yl)methoxy)isoquinolin- | (M + 1). | 5.6 Hz, 2H), 7.57 (s, 1H), 7.45 (d, J = |
| | 1-amine | | 8.7 Hz, 1H), 7.27 (dd, J = 9.2, 2.6 Hz, |
| | | | 1H), 7.20 (d, J = 5.8 Hz, 1H), 5.08 (s, |
| | | | 2H), 3.75-3.69 (m, 1H), 1.03-0.94 (m, |
| | | | 4H), NH not observed. |
| 290 | 6-((2-oxaspiro[3.3]heptan- | 368.2 | ¹H NMR (400 MHZ, DMSO-d₆) δ 9.40 |
| | 5-yl)oxy)-N-(6- | (M + 1), | (s, 1H), 8.88 (d, J = 2.7 Hz, 1H), 8.47 |
| | chloropyridin-3- | 370.2 | (d, J = 9.2 Hz, 1H), 8.43 (dd, J = 8.8, |
| | yl)isoquinolin-1-amine | (M + 1). | 2.8 Hz, 1H), 7.97 (d, J = 5.8 Hz, 1H), |
| | | | 7.45 (d, J = 8.7 Hz, 1H), 7.35 (dd, J = |
| | | | 9.2, 2.6 Hz, 1H), 7.28 (d, J = 2.5 Hz, |
| | | | 1H), 7.20 (d, J = 5.8 Hz, 1H), 5.07 (d, |
| | | | J = 6.0 Hz, 1H), 4.91 (t, J = 7.3 Hz, |
| | | | 1H), 4.68 (d, J = 7.0 Hz, 1H), 4.56 (d, |
| | | | J = 6.1 Hz, 1H), 4.53 (d, J = 7.0 Hz, |
| | | | 1H), 2.46-2.43 (m, 1H), 2.18-2.12 (m, |
| | | | 1H), 1.96-1.90 (m, 1H), 1.77-1.69 (m, 1H). |
| 291 | 6-((5-chloro-1-methyl- | 400.0 | ¹H NMR (400 MHZ, DMSO-d₆) δ |
| | 1H-pyrazol-4-yl)methoxy)- | (M + 1), | 10.23 (brs, 1H), 8.77 (d, J = 2.2 Hz, |
| | N-(6-chloropyridin-3- | 402.0 | 1H), 8.53 (d, J = 9.1 Hz, 1H), 8.26 |
| | yl)isoquinolin-1-amine | (M + 1). | (dd, J= 8.8, 1.2 Hz, 1H), 7.82-7.79 |
| | | | (m, 2H), 7.59-7.55 (m, 2H), 7.39 (d, |
| | | | J = 8.5 Hz, 1H), 7.27 (d, J = 6.3 Hz, |
| | | | 1H), 5.11 (s, 2H), 3.83 (s, 3H). |
| 292 | 6-((3-chloro-1-methyl- | 400.2 | ¹H NMR (400 MHZ, DMSO-d₆) δ 9.39 |
| | 1H-pyrazol-4- | (M + 1), | (s, 1H), 8.88 (d, J = 2.8 Hz, 1H), 8.45- |
| | yl)methoxy)-N-(6- | 402.2 | 8.43 (m, 2H), 8.02 (s, 1H), 7.98 (d, |
| | chloropyridin-3- | (M + 1). | J = 5.8 Hz, 1H), 7.44 (t, J = 6.0 Hz, 2H), |
| | yl)isoquinolin-1-amine | | 7.28 (dd, J = 9.3, 2.5 Hz, 1H), 7.20 (d, |
| | | | J = 5.8 Hz, 1H), 5.05 (s, 2H), 3.83 (s, |
| | | | 3H). |
| 293 | 3-(((1-((6-chloropyridin- | 404.0 | ¹H NMR (400 MHZ, DMSO-d₆) δ |
| | 3-yl)amino)isoquinolin- | (M + 1), | 10.14 (brs, 1H), 8.80 (d, J = 2.2 Hz, |
| | 6-yl)oxy)methyl)-3- | 406.0 | 1H), 8.55 (d, J = 9.1 Hz, 1H), 8.31- |
| | methylthietane 1,1-dioxide | (M + 1). | 8.28 (m, 1H), 7.85-7.83 (m, 1H), 7.58 |
| | | | (d, J = 8.7 Hz, 1H), 7.45-7.41 (m, 2H), |
| | | | 7.26 (d, J = 6.2 Hz, 1H), 4.27 (d, J = |
| | | | 11.6 Hz, 4H), 4.04 (d, J = 14.3 Hz, |
| | | | 2H), 1.55 (s, 3H). |
| 294 | 1-(((1-((6-chloropyridin- | 419.0 | ¹H NMR (400 MHZ, DMSO-d₆) δ 9.42 |
| | 3-yl)amino)isoquinolin- | (M + 1), | (s, 1H), 8.89 (d, J = 2.7 Hz, 1H), 8.49- |
| | 6-yl)oxy)methyl)cyclobutane- | 421.0 | 8.43 (m, 2H), 7.99 (d, J = 5.8 Hz, 1H), |
| | 1-sulfonamide | (M + 1). | 7.46 (d, J = 8.8 Hz, 1H), 7.40-7.37 (m, |
| | | | 2H), 7.21 (d, J = 5.8 Hz, 1H), 6.97 (s, |
| | | | 2H), 4.43 (s, 2H), 2.62-2.59 (m, 2H), |
| | | | 2.33-2.31 (m, 2H), 2.06-1.98 (m, 2H). |
| 295 | 6-((1-benzyl-1H-pyrazol- | 442.2 | ¹H NMR (400 MHZ, DMSO-d₆) δ 9.40 |
| | 4-yl)methoxy)-N-(6- | (M + 1), | (s, 1H), 8.87 (s, 1H), 8.43-8.42 (m, |
| | chloropyridin-3- | 444.2 | 2H), 8.00 (s, 1H), 7.96 (d, J = 4.7 Hz, |
| | yl)isoquinolin-1-amine | (M + 1). | 1H), 7.62 (s, 1H), 7.46-7.25 (m, 9H), |
| | | | 5.33 (s, 2H), 5.12 (s, 2H). |
| 296 | N-(6-chloropyridin-3- | 394.2 | ¹H NMR (400 MHZ, DMSO-d₆) δ 9.38 |
| | yl)-6-((1-(1-methyl-1H- | (M + 1), | (s, 1H), 8.88 (d, J = 2.8 Hz, 1H), 8.43 |
| | pyrazol-4-yl)propan-2- | 396.2 | (dd, J = 8.4, 3.7 Hz, 2H), 7.95 (d, J = |
| | yl)oxy)isoquinolin-1- | (M + 1). | 5.7 Hz, 1H), 7.57 (s, 1H), 7.45 (d, J = |
| | amine | | 8.5 Hz, 1H), 7.30 (dd, J = 17.3, 8.5 |
| | | | Hz, 3H), 7.18 (d, J = 6.2 Hz, 1H), |
| | | | 4.81-4.77 (m, 1H), 3.78 (s, 3H), 2.81 |
| | | | (qd, J = 13.4, 5.8 Hz, 2H), 1.30 (d, J = |
| | | | 5.9 Hz, 3H). |
| 297 | (R)-N-(6-chloropyridin- | 342.2 | ¹H NMR (400 MHZ, DMSO-d₆) δ |
| | 3-yl)-6-((tetrahydrofuran- | (M + 1), | 10.56 (s, 1H), 8.73 (d, J = 2.7 Hz, |
| | 3-yl)oxy)isoquinolin-1- | 344.2 | 1H), 8.57 (d, J = 9.1 Hz, 1H), 8.20 |
| | amine trifluoroacetate | (M + 1) | (dd, J = 8.6, 2.7 Hz, 1H), 7.74 (d, J = |
| | | | 6.5 Hz, 1H), 7.65 (d, J = 8.6 Hz, 1H), |
| | | | 7.46-7.41 (m, 2H), 7.31 (d, J = 6.6 Hz |
| | | | 1H), 5.29-5.26 (m, 1H), 3.99 (dd, J = |
| | | | 10.4, 4, 4 Hz, 1H), 3.91-3.87 (m, 2H), |
| | | | 3.81 (td, J = 8.4, 4.7 Hz, 1H), 2.37 |
| | | | (dtd, J = 13.9, 7.9, 6.1 Hz, 1H), 2.08- |
| | | | 2.02 (m, 1H); ¹⁹F NMR (376 MHZ, |
| | | | DMSO-d₆) δ −74.5 (s). |
| 298 | (S)-N-(6-chloropyridin- | 342.2 | ¹H NMR (400 MHZ, DMSO-d₆) δ |
| | 3-yl)-6-((tetrahydrofuran- | (M + 1), | 10.45 (s, 1H), 8.74 (d, J = 2.7 Hz, |
| | 3-yl)oxy)isoquinolin-1- | 344.2 | 1H), 8.55 (d, J = 9.1 Hz, 1H), 8.21 |
| | amine trifluoroacetate | (M + 1) | (dd, J = 8.6, 2.6 Hz, 1H), 7.76 (d, J = |
| | | | 6.4 Hz, 1H), 7.64 (d, J = 8.6 Hz, 1H), |
| | | | 7.44-7.41 (m, 2H), 7.30 (d, J = 6.5 Hz, |
| | | | 1H), 5.27 (td, J = 4.3, 1.8 Hz, 1H), |
| | | | 3.99 (dd, J = 10.3, 4.5 Hz, 1H), 3.89 |
| | | | (dd, J = 9.5, 5.2 Hz, 2H), 3.81 (td, J = |
| | | | 8.3, 4.7 Hz, 1H), 2.37 (dtd, J = 13.9, |
| | | | 7.9, 6.1 Hz, 1H), 2.08-2.02 (m, 1H); |
| | | | ¹⁹F NMR (376 MHZ, DMSO-d₆) δ −74.4 (s). |
| 299 | N-(6-chloropyridin-3- | 386.0 | ¹H NMR (400 MHz, DMSO-d₆) δ 9.39 |
| | yl)-6-((2,2-dimethyl-1,3- | (M + 1), | (s, 1H), 8.88 (d, J = 2.7 Hz, 1H), 8.45- |
| | dioxolan-4- | 388.0 | 8.41 (m, 2H), 7.96 (d, J = 5.8 Hz, 1H), |
| | yl)methoxy)isoquinolin- | (M + 1). | 7.45 (d, J = 8.7 Hz, 1H), 7.33-7.28 (m, |
| | 1-amine | | 2H), 7.18 (d, J = 5.8 Hz, 1H), 4.50- |
| | | | 4.46 (m, 1H), 4.22-4.18 (m, 1H), 4.16- |
| | | | 4.12 (m, 2H), 3.81 (dd, J = 8.4, 6.3 |
| | | | Hz, 1H), 1.38 (s, 3H), 1.33 (s, 3H). |
| 300 | 3-(((1-((6-chloropyridin- | 389.9 | ¹H NMR (400 MHZ, DMSO-d₆) δ 9.43 |
| | 3-yl)amino)isoquinolin- | (M + 1), | (s, 1H), 8.88 (d, J = 2.8 Hz, 1H), 8.46 |
| | 6-yl)oxy)methyl)thietane | 392.0 | (d, J = 9.2 Hz, 1H), 8.43 (dd, J = 8.8, |
| | 1,1-dioxide | (M + 1). | 2.8 Hz, 1H), 7.97 (d, J = 5.8 Hz, 1H), |
| | | | 7.45 (d, J = 8.7 Hz, 1H), 7.32 (d, J = |
| | | | 2.4 Hz, 1H), 7.29 (dd, J = 9.1, 2.5 Hz, |
| | | | 1H), 7.19 (d, J = 5.8 Hz, 1H), 4.37 |
| | | | (dd, J = 14.6, 9.7 Hz, 2H), 4.32 (d, J = |
| | | | 7.0 Hz, 2H), 4.11 (d, J = 6.1 Hz, 1H), |
| | | | 4.08 (d, J = 6.0 Hz, 1H), 3.05 (dtt, J = |
| | | | 12.9, 9.6, 6.5 Hz, 1H). |
| 301 | N-(6-chloropyridin-3- | 369.0 | ¹H NMR (400 MHZ, DMSO-d₆) δ 9.67 |
| | yl)-6-(isothiazol-4- | (M + 1), | (br s, 1H), 9.20 (s, 1H), 8.86 (d, J = |
| | ylmethoxy)isoquinolin- | 371.0 | 1.8 Hz, 1H), 8.75 (s, 1H), 8.51 (d, J = |
| | 1-amine | (M + 1). | 9.2 Hz, 1H), 8.38 (dt, J = 8.6, 0.9 Hz, |
| | | | 1H), 7.93 (d, J = 5.9 Hz, 1H), 7.51- |
| | | | 7.47 (m, 2H), 7.40-7.37 (m, 1H), 7.23 |
| | | | (d, J = 5.9 Hz, 1H), 5.42 (s, 2H). |

### Example 302 and 303

### Synthesis of (R)—N-(6-chloropyridin-3-yl)-6-((2-methyltetrahydrofuran-2-yl)methoxy)isoquinolin-1-amine and (S)—N-(6-chloropyridin-3-yl)-6-((2-methyltetrahydrofuran-2-yl)methoxy)isoquinolin-1-amine

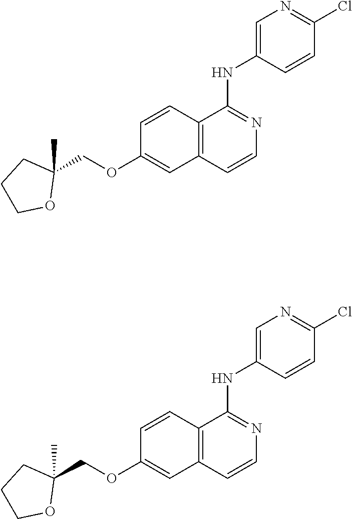 [SMILES: C[C@]1(COc2ccc3c(Nc4ccc(Cl)nc4)nccc3c2)CCCO1 ; C[C@@]1(COc2ccc3c(Nc4ccc(Cl)nc4)nccc3c2)CCCO1]

### Step 1. Preparation of N-(6-chloropyridin-3-yl)-6-((2-methyltetrahydrofuran-2-yl)methoxy)isoquinolin-1-amine

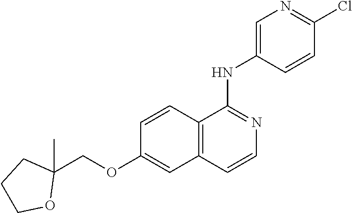 [SMILES: CC1(COc2ccc3c(Nc4ccc(Cl)nc4)nccc3c2)CCCO1]

Following the procedure as described for EXAMPLE 279, Step 2, and making variations as required to replace (1-(difluoromethyl)-1H-pyrazol-4-yl)methanol with (2-methyloxolan-2-yl)methanol, the title compound was obtained as a colorless solid (0.075 g, 55% yield): MS (ES+) m/z 370.2 (M+1), 372.2 (M+1).

### Step 2. Preparation of (R)—N-(6-chloropyridin-3-yl)-6-((2-methyltetrahydrofuran-2-yl)methoxy)isoquinolin-1-amine and (S)—N-(6-chloropyridin-3-yl)-6-((2-methyltetrahydrofuran-2-yl)methoxy)isoquinolin-1-amine

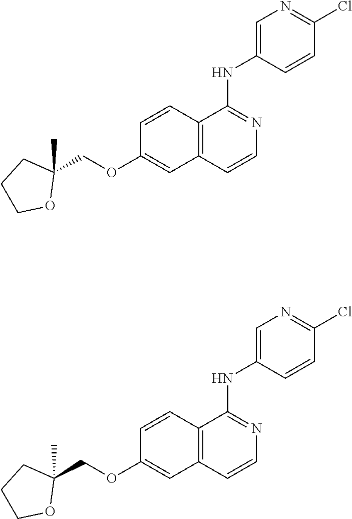 [SMILES: C[C@]1(COc2ccc3c(Nc4ccc(Cl)nc4)nccc3c2)CCCO1 ; C[C@@]1(COc2ccc3c(Nc4ccc(Cl)nc4)nccc3c2)CCCO1]

Resolution of the enantiomers of N-(6-chloropyridin-3-yl)-6-((2-methyltetrahydrofuran-2-yl)methoxy)isoquinolin-1-amine by chiral SFC (LuxCel-2 250×10 mm, 5 μm column), eluting with 45% of methanol (containing 10 mM ammonium formate) in supercritical carbon dioxide, afforded the title compounds as single enantiomers as colorless solids. First eluting enantiomer (0.032 g, 42% yield): ¹H NMR (400 MHz, DMSO-d₆) δ9.38 (s, 1H), 8.88 (d, J=2.8 Hz, 1H), 8.43 (dt, J=9.1, 2.5 Hz, 2H), 7.95 (d, J=5.8 Hz, 1H), 7.43 (d, J=8.7 Hz, 1H), 7.29-7.28 (m, 2H), 7.17 (d, J=5.8 Hz, 1H), 3.97 (d, J=1.6 Hz, 2H), 3.78 (dd, J=6.2, 3.6 Hz, 2H), 2.02-1.91 (m, 3H), 1.73-1.69 (m, 1H), 1.29 (s, 3H); MS (ES+) m/z 370.2 (M+1), 372.2 (M+1). Second eluting enantiomer (0.033 g, 44% yield): ¹H NMR (400 MHz, DMSO-d₆) δ 9.38 (s, 1H), 8.88 (d, J=0.2 Hz, 1H), 8.45-8.42 (m, 2H), 7.96 (d, J=5.9 Hz, 1H), 7.44 (d, J=8.6 Hz, 1H), 7.30-7.28 (m, 2H), 7.17 (d, J=5.4 Hz, 1H), 3.98 (s, 2H), 3.79 (dt, J=2.2, 1.1 Hz, 2H), 1.99-1.92 (m, 3H), 1.72-1.69 (m, 1H), 1.29 (s, 3H); MS (ES+) m/z 370.2 (M+1), 372.2 (M+1).

### Example 304

### Synthesis of 6-(((1H-pyrazol-4-yl)oxy)methyl)-N-(6-chloropyridin-3-yl)isoquinolin-1-amine

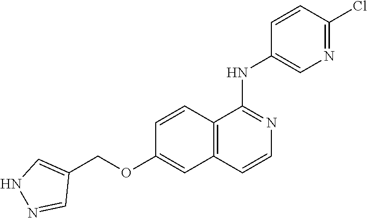 [SMILES: Clc1ccc(Nc2nccc3cc(OCc4cn[nH]c4)ccc23)cn1]

### Step 1. Preparation of methyl 1-((6-chloropyridin-3-yl)((2-(trimethylsilyl)ethoxy)methyl)amino)isoquinoline-6-carboxylate

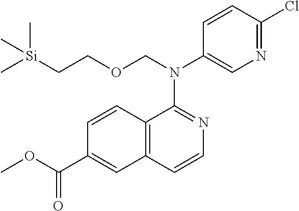 [SMILES: COC(=O)c1ccc2c(N(COCC[Si](C)(C)C)c3ccc(Cl)nc3)nccc2c1]

Following the procedure as described for EXAMPLE 76, Step 2, making variations as required to replace N-(6-chloropyridin-3-yl)-6-fluoroisoquinolin-1-amine with methyl 1-((6-chloropyridin-3-yl)amino)isoquinoline-6-carboxylate, the title compound was obtained as a brown liquid (0.318 g, 33% yield): MS (ES+) m/z 444.5 (M+1), 446.5 (M+1).

### Step 2. Preparation of (1-((6-chloropyridin-3-yl)((2-(trimethylsilyl)ethoxy)methyl)amino)isoquinolin-6-yl)methanol

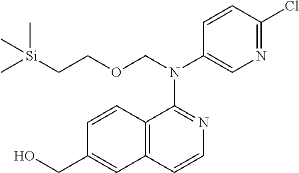 [SMILES: C[Si](C)(C)CCOCN(c1ccc(Cl)nc1)c1nccc2cc(CO)ccc12]

To a solution of methyl 1-((6-chloropyridin-3-yl)((2-(trimethylsilyl)ethoxy)methyl)amino)isoquinoline-6-carboxylate (0.318 g, 0.645 mmol) in tetrahydrofuran (4 mL) was added a 4 M solution of lithium borohydride (0.64 mL, 2.58 mmol) in tetrahydrofuran and the reaction mixture was stirred at ambient temperature for 2 h. The mixture was diluted with ethyl acetate (30 mL) and washed with saturated sodium bicarbonate (30 mL) and brine (30 mL). The organic phase was dried over anhydrous sodium sulfate and filtered. The filtrate was concentrated in vacuo to give the title compound as a yellowish solid (0.257 g, 96% yield): MS (ES+) m/z 416.4 (M+1), 418.4 (M+1).

### Step 3. Preparation of 6-(chloromethyl)-N-(6-chloropyridin-3-yl)-N-((2-(trimethylsilyl)ethoxy)methyl)isoquinolin-1-amine

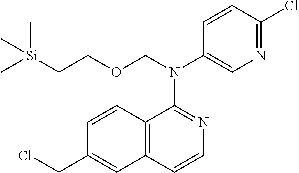 [SMILES: C[Si](C)(C)CCOCN(c1ccc(Cl)nc1)c1nccc2cc(CCl)ccc12]

To a solution of (1-((6-chloropyridin-3-yl)((2-(trimethylsilyl)ethoxy)methyl)amino)isoquinolin-6-yl)methanol (0.257 g, 0.618 mmol) in dichloromethane (4 mL) was added triethylamine (0.26 mL, 1.85 mmol) and p-toluenesulfonyl chloride (0.141 g, 0.741 mmol) and the reaction mixture was stirred at ambient temperature for 16 h. The mixture was diluted with ethyl acetate (30 mL) and washed with saturated sodium bicarbonate (30 mL) and brine (30 mL). The organic phase was dried over anhydrous sodium sulfate and filtered. The filtrate was concentrated in vacuo to give the title compound as a yellowish solid (0.305 g, quantitative yield) and it was used in the next step without purification: MS (ES+) m/z 434.5 (M+1), 436.5 (M+1), 438.5 (M+1).

### Step 4. Preparation of 6-(((1H-pyrazol-4-yl)oxy)methyl)-N-(6-chloropyridin-3-yl)isoquinolin-1-amine

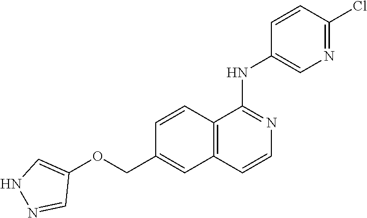 [SMILES: Clc1ccc(Nc2nccc3cc(COc4cn[nH]c4)ccc23)cn1]

To a solution of 6-(chloromethyl)-N-(6-chloropyridin-3-yl)-N-((2-(trimethylsilyl)ethoxy)methyl)isoquinolin-1-amine (0.305 g, 0.702 mmol) in N,N-dimethylformamide (3 mL) was added 1H-pyrazol-4-ol (0.077 g, 0.916 mmol) and potassium carbonate (0.291 g, 2.11 mmol). The reaction was stirred at ambient temperature for 16 h. The mixture was diluted with ethyl acetate (30 mL) and washed with saturated sodium bicarbonate (30 mL) and brine (30 mL). The organic phase was dried over anhydrous sodium sulfate and filtered. The filtrate was concentrated in vacuo to give a residue. To the residue was added dichloromethane (2 mL) and trifluoroacetic acid (1 mL, 13.1 mmol). The resulting mixture was stirred at ambient temperature for 2 h, and then concentrated in vacuo to give a residue. The obtained residue was purified by reverse-phase preparative HPLC (Phenomenex Gemini-NX C18 150 mm×30 mm, 5 μm column), eluting with a gradient of 15 to 50% of acetonitrile in water (containing 0.5% of formic acid), to provide the title compound as a colorless solid (0.011 g, 4% yield): ¹H NMR (400 MHz, DMSO-d₆) δ 12.41 (s, 1H), 9.50 (s, 1H), 8.91-8.90 (m, 1H), 8.54 (d, J=8.8 Hz, 1H), 8.45 (dd, J=8.8, 2.9 Hz, 1H), 8.05 (d, J=5.7 Hz, 1H), 7.90 (d, J=0.7 Hz, 1H), 7.71-7.68 (m, 1H), 7.55-7.46 (m, 2H), 7.38-7.29 (m, 2H), 5.15 (s, 2H); MS (ES+) m/z 352.4 (M+1), 354.2 (M+1).

### Example 305

### Synthesis of 1-(4-((1-((6-chloropyridin-3-yl)amino)isoquinolin-6-yl)oxy)piperidin-1-yl)ethan-1-one

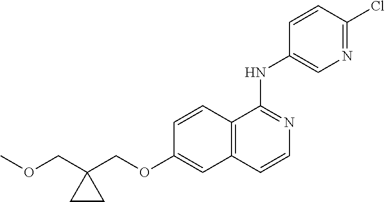 [SMILES: COCC1(COc2ccc3c(Nc4ccc(Cl)nc4)nccc3c2)CC1]

### Step 1. Preparation of ethyl 1-(((1-((6-chloropyridin-3-yl)((2-(trimethylsilyl)ethoxy)methyl)amino)isoquinolin-6-yl)oxy)methyl)cyclopropane-1-carboxylate

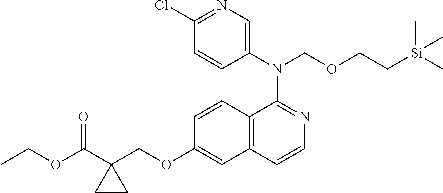 [SMILES: CCOC(=O)C1(COc2ccc3c(N(COCC[Si](C)(C)C)c4ccc(Cl)nc4)nccc3c2)CC1]

Following the procedure as described for EXAMPLES 264 and 265, Step 1, and making variations as required to replace 4-hydroxycyclohexanone with ethyl 1-(hydroxymethyl)cyclopropane-1-carboxylate, the title compound was obtained as a colorless solid (0.274 g, 56% yield): ¹H NMR (400 MHz, CDCl₃) δ8.32 (dd, J=5.7, 1.6 Hz, 1H), 8.05 (t, J=3.0 Hz, 1H), 7.71-7.68 (m, 1H), 7.54-7.53 (m, 1H), 7.47-7.45 (m, 1H), 7.18-7.15 (m, 1H), 7.14-7.10 (m, 2H), 5.41 (s, 2H), 4.28 (s, 1H), 4.21-4.16 (m, 2H), 3.59-3.54 (m, 3H), 1.51 (t, J=7.1 Hz, 2H), 1.28 (t, J=7.2 Hz, 3H), 0.92-0.87 (m, 4H), −0.09 (s, 9H); MS (ES+) m/z 528.6 (M+1), 530.6 (M+1).

### Step 2. Preparation of (1-(((1-((6-chloropyridin-3-yl)((2-(trimethylsilyl)ethoxy)methyl)amino)isoquinolin-6-yl)oxy)methyl)cyclopropyl)methanol

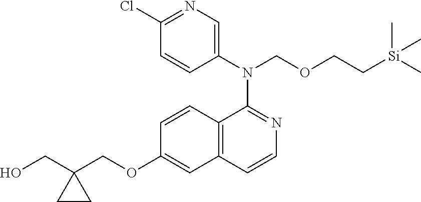 [SMILES: C[Si](C)(C)CCOCN(c1ccc(Cl)nc1)c1nccc2cc(OCC3(CO)CC3)ccc12]

To a solution of ethyl 1-(((1-((6-chloropyridin-3-yl)((2-(trimethylsilyl)ethoxy)methyl)amino)isoquinolin-6-yl)oxy)methyl)cyclopropane-1-carboxylate (0.274 g, 0.519 mmol) in tetrahydrofuran (4 mL) was added a 4 M solution of lithium borohydride (0.26 mL, 1.04 mmol) in tetrahydrofuran and the reaction mixture was stirred at ambient temperature for 16 h. The mixture was then diluted with ethyl acetate (30 mL), and washed with saturated aqueous sodium bicarbonate (30 mL) and brine (30 mL). The organic phase was dried over anhydrous sodium sulfate and filtered. The filtrate was concentrated in vacuo to give a residue. The obtained residue was purified by silica gel column chromatography, eluting with a gradient of 0 to 80% of ethyl acetate in heptane, to provide the title compound as a colourless solid (0.088 g, 25% yield): MS (ES+) m/z 486.6 (M+1), 488.6 (M+1).

### Step 3. Preparation of 1-(4-((1-((6-chloropyridin-3-yl)amino)isoquinolin-6-yl)oxy)piperidin-1-yl)ethan-1-one

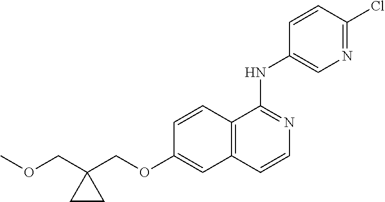 [SMILES: COCC1(COc2ccc3c(Nc4ccc(Cl)nc4)nccc3c2)CC1]

To a solution of (1-(((1-((6-chloropyridin-3-yl)((2-(trimethylsilyl)ethoxy)methyl)amino)isoquinolin-6-yl)oxy)methyl)cyclopropyl)methanol (0.088 g, 0.181 mmol) in tetrahydrofuran (2 mL) was added sodium hydride (60% dispersion in mineral oil, 0.029 g, 0.724 mmol). The mixture was stirred for 2 minutes, and then iodomethane (0.023 mL, 0.362 mmol) was added to it. The reaction mixture was stirred at ambient temperature for 30 minutes and then concentrated in vacuo to give a residue. To it was added dichloromethane (2 mL) and trifluoroacetic acid (1.0 mL, 13.1 mmol), and the resulting mixture was stirred at ambient temperature for 2 h. The mixture was concentrated in vacuo and the obtained residue was purified by silica gel column chromatography, eluting with a gradient of 0 to 100% of ethyl acetate in heptane, to provide the title compound as a colourless solid (0.010 g, 15% yield): ¹H NMR (400 MHz, DMSO-d₆) δ9.39 (s, 1H), 8.89 (d, J=2.8 Hz, 1H), 8.44 (dd, J=8.8, 2.9 Hz, 2H), 7.96 (d, J=5.8 Hz, 1H), 7.45 (d, J=8.7 Hz, 1H), 7.32-7.27 (m, 2H), 7.17 (d, J=5.8 Hz, 1H), 4.02 (s, 2H), 3.37 (s, 2H), 3.27 (s, 3H), 0.66-0.57 (m, 4H); MS (ES+) m/z 370.2 (M+1), 372.2 (M+1).

### Example 306

### Synthesis of N-(6-chloropyridin-3-yl)-6-((3-(methoxymethyl)oxetan-3-yl)methoxy)isoquinolin-1-amine

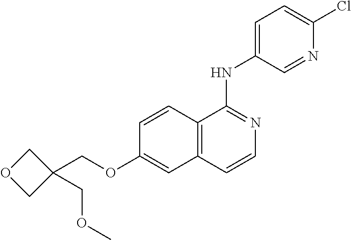 [SMILES: COCC1(COc2ccc3c(Nc4ccc(Cl)nc4)nccc3c2)COC1]

### Step 1. Preparation of (3-(((1-((6-chloropyridin-3-yl)((2-(trimethylsilyl)ethoxy)methyl)amino)isoquinolin-6-yl)oxy)methyl)oxetan-3-yl)methanol

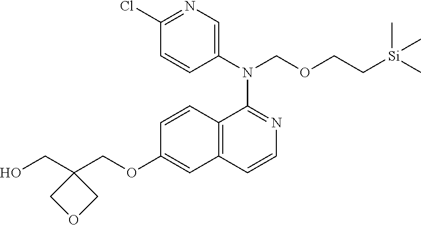 [SMILES: C[Si](C)(C)CCOCN(c1ccc(Cl)nc1)c1nccc2cc(OCC3(CO)COC3)ccc12]

Following the procedure as described for EXAMPLES 264 and 265, Step 1, and making variations as required to replace 4-hydroxycyclohexanone with oxetane-3,3-diyldimethanol, the title compound was obtained as a colorless solid (0.125 g, 39% yield): ¹H NMR (400 MHz, DMSO-d₆) δ8.31 (d, J=5.7 Hz, 1H), 7.91-7.90 (m, 1H), 7.66 (dd, J=14.0, 7.5 Hz, 2H), 7.52 (d, J=2.6 Hz, 1H), 7.31 (d, J=8.8 Hz, 1H), 7.21 (dd, J=9.3, 2.5 Hz, 1H), 7.15 (dd, J=8.8, 3.1 Hz, 1H), 5.38-5.36 (m, 2H), 5.07-5.04 (m, 1H), 4.44 (q, J=6.2 Hz, 4H), 4.31 (s, 2H), 3.75-3.73 (m, 2H), 3.54 (t, J=8.0 Hz, 2H), 0.81-0.77 (m, 2H), −0.14 (s, 9H); MS (ES+) m/z 502.2 (M+1), 504.2 (M+1).

### Step 2. Preparation of N-(6-chloropyridin-3-yl)-6-((3-(methoxymethyl)oxetan-3-yl)methoxy)isoquinolin-1-amine

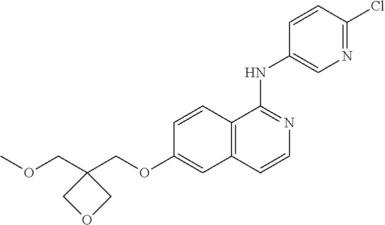 [SMILES: COCC1(COc2ccc3c(Nc4ccc(Cl)nc4)nccc3c2)COC1]

Following the procedure as described for EXAMPLE 305, Step 3, and making variations as required to replace (1-(((1-((6-chloropyridin-3-yl)((2-(trimethylsilyl)ethoxy)methyl)amino)isoquinolin-6-yl)oxy)methyl)cyclopropyl)methanol with (3-(((1-((6-chloropyridin-3-yl)((2-(trimethylsilyl)ethoxy)methyl)amino)isoquinolin-6-yl)oxy)methyl)oxetan-3-yl)methanol, the title compound was obtained as a colorless solid (0.019 g, 39% yield): ¹H NMR (400 MHz, DMSO-d₆) δ9.51 (br s, 1H), 8.88-8.87 (m, 1H), 8.49-8.47 (m, 1H), 8.43-8.40 (m, 1H), 7.96-7.93 (m, 1H), 7.49-7.47 (m, 1H), 7.40-7.34 (m, 2H), 7.23-7.21 (m, 1H), 4.52 (d, J=6.0 Hz, 2H), 4.46 (d, J=6.0 Hz, 2H), 4.33 (s, 2H), 3.70 (s, 2H), 3.34 (s, 3H); MS (ES+) m/z 386.0 (M+1), 388.0 (M+1).

### Example 307

### Synthesis of N-(6-chloropyridin-3-yl)-6-((1-methyl-1H-1,2,4-triazol-3-yl)methoxy)isoquinolin-1-amine

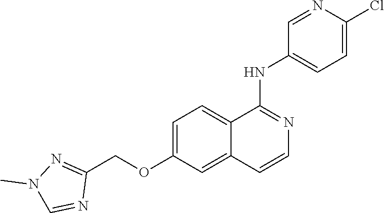 [SMILES: Cn1cnc(COc2ccc3c(Nc4ccc(Cl)nc4)nccc3c2)n1]

A mixture of 3-(chloromethyl)-1-methyl-1H-1,2,4-triazole hydrochloride (0.200 g, 1.19 mmol), 1-chloroisoquinolin-6-ol (0.144 g, 0.802 mmol), and potassium carbonate (0.439 g, 3.17 mmol) in N,N-dimethylformamide (5 mL) was stirred at 90° C. for 12 h. After cooling to ambient temperature, the reaction mixture was poured into water (10 mL). The mixture was extracted with ethyl acetate (3×20 mL). The combined organic layers were washed with brine (3×10 mL), dried over anhydrous sodium sulfate, and filtered. The filtrate was concentrated in vacuo to provide a residue (0.100 g). To it was added 6-chloropyridin-3-amine (0.0468 g, 0.364 mmol), [(2-di-tert-butylphosphino-2′,4′,6′-triisopropyl-1,1′-biphenyl)-2-(2′-amino-1,1′-biphenyl)]palladium(II) methanesulfonate (0.029 g, 0.0364 mmol) cesium carbonate (0.356 g, 1.09 mmol), and 2-methylbutan-2-ol (5 mL), and the mixture was stirred at 90° C. for 12 h. After cooling to ambient temperature, the reaction mixture was concentrated in vacuo. The obtained residue was purified by reverse-phase preparative HPLC (Waters XBridge 150 mm×25 mm, 5 μm column), eluting with a gradient of 25% to 55% of acetonitrile in water (containing 10 mM of ammonium carbonate), to provide the title compound as a yellowish solid (0.010 g, 3% yield): ¹H NMR (400 MHz, CD₃OD) δ 8.72 (d, J=2.7 Hz, 1H), 8.43 (d, J=0.2 Hz, 1H), 8.28-8.25 (m, 2H), 7.92-7.91 (m, 1H), 7.40-7.38 (m, 1H), 7.36-7.35 (m, 1H), 7.30-7.27 (m, 1H), 7.19-7.17 (m, 1H), 5.27 (s, 2H), 3.96 (s, 3H), NH not observed; MS (ES+) m/z 367.3 (M+1), 369.3 (M+1).

### Example 308

### Synthesis of N-(2-methylpyrimidin-5-yl)-6-(pyrimidin-5-ylmethoxy)isoquinolin-1-amine

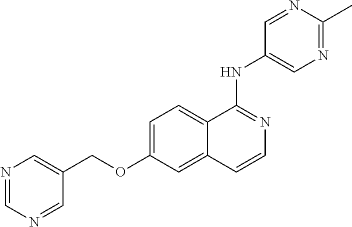 [SMILES: Cc1ncc(Nc2nccc3cc(OCc4cncnc4)ccc23)cn1]

To a solution of 5-(chloromethyl)pyrimidine hydrochloride (0.200 g, 1.21 mmol) in N,N-dimethylformamide (5 mL) was added potassium carbonate (0.492 g, 3.56 mmol) and 1-chloroisoquinolin-6-ol (0.160 g, 0.890 mmol) at ambient temperature. The reaction mixture was heated up to 90° C. for 12 h. After cooling to ambient temperature, the reaction mixture was poured into water (50 mL). The mixture was extracted with ethyl acetate (3×50 mL). The combined organic layers were washed with brine (3×100 mL), dried over anhydrous sodium sulfate, and filtered. The filtrate was concentrated in vacuo to provide a residue (0.193 g). To it was added 2-methylpyrimidin-5-amine (0.116 g, 1.06 mmol), [(2-di-tert-butylphosphino-2′,4′,6′-triisopropyl-1,1′-biphenyl)-2-(2′-amino-1,1′-biphenyl)]palladium(II) methanesulfonate (0.058 g, 0.0730 mmol), cesium carbonate (0.695 g, 2.13 mmol), and 2-methylbutan-2-ol (6 mL) and the reaction mixture was stirred at 70° C. for 12 h. After cooling to ambient temperature, the reaction mixture was concentrated in vacuo. The obtained residue was purified by reverse-phase preparative HPLC (Phenomenex Luna C18 150×25 mm, 10 μm column), eluting with a gradient of 2% to 32% of acetonitrile in water (containing 0.225% of formic acid), to provide the title compound as a yellow solid (0.043 g, 14% yield): ¹H NMR (400 MHz, CD₃OD) δ 9.16 (s, 1H), 9.13 (s, 2H), 8.97 (s, 2H), 8.30 (d, J=9.0 Hz, 1H), 7.94 (d, J=5.8 Hz, 1H), 7.36-7.32 (m, 2H), 7.20 (d, J=5.9 Hz, 1H), 5.34 (s, 2H), 2.66 (s, 3H), NH not observed; MS (ES+) m/z 345.2 (M+1).

### Example 309

### Synthesis of 1-(3-(((1-((6-chloropyridin-3-yl)amino)isoquinolin-6-yl)oxy)methyl)azetidin-1-yl)ethan-1-one

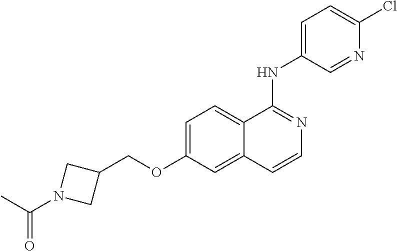 [SMILES: CC(=O)N1CC(COc2ccc3c(Nc4ccc(Cl)nc4)nccc3c2)C1]

### Step 1. Preparation of tert-butyl 3-(((1-chloroisoquinolin-6-yl)oxy)methyl)azetidine-1-carboxylate

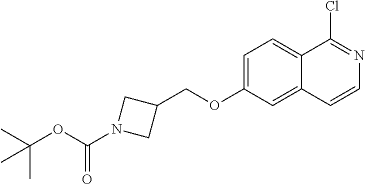 [SMILES: CC(C)(C)OC(=O)N1CC(COc2ccc3c(Cl)nccc3c2)C1]

Following the procedure as described for EXAMPLE 36, Step 2, and making variations as required to replace (3-methyloxetan-3-yl)methyl 4-methylbenzenesulfonate with tert-butyl 3-(iodomethyl)azetidine-1-carboxylate, the title compound was obtained as a colorless solid (1.20 g, quantitative yield): ¹H NMR (400 MHz, CDCl₃) δ 8.23 (d, J=9.4 Hz, 1H), 8.19 (d, J=5.4 Hz, 1H), 7.47 (d, J=5.6 Hz, 1H), 7.31-7.27 (m, 1H), 7.09 (d, J=2.4 Hz, 1H), 4.24 (d, J=6.6 Hz, 2H), 4.14 (t, J=8.6 Hz, 2H), 3.84 (dd, J=8.8, 5.2 Hz, 2H), 3.11-3.00 (m, 1H), 1.46 (s, 9H).

### Step 2. Preparation of 6-(azetidin-3-ylmethoxy)-1-chloroisoquinoline hydrochloride

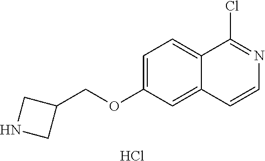 [SMILES: Clc1nccc2cc(OCC3CNC3)ccc12 ; Cl]

A mixture of tert-butyl 3-(((1-chloroisoquinolin-6-yl)oxy)methyl)azetidine-1-carboxylate (1.15 g, 3.30 mmol) in a 4 M solution of hydrogen chloride in ethyl acetate (10 mL, 40 mmol) was stirred at ambient temperature for 30 minutes. The solid was filtered off and dried to provide the title compound as a colorless solid (0.9 g, 96% yield): ¹H NMR (400 MHz, DMSO-d₆) δ 9.61-9.33 (m, 2H), 8.47 (s, 1H), 8.21 (d, J=5.6 Hz, 1H), 8.18 (d, J=8.8 Hz, 1H), 7.78 (d, J=5.6 Hz, 1H), 7.57-7.51 (m, 1H), 4.58-4.51 (m, 1H), 4.36 (d, J=6.0 Hz, 2H), 4.16-4.06 (m, 1H), 4.03-3.90 (m, 1H), 3.90-3.80 (m, 1H), 3.31-3.21 (m, 1H).

### Step 3. Preparation of 1-(3-(((1-chloroisoquinolin-6-yl)oxy)methyl)azetidin-1-yl)ethan-1-one

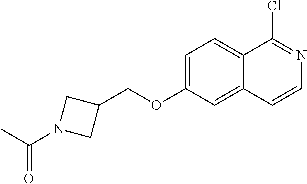 [SMILES: CC(=O)N1CC(COc2ccc3c(Cl)nccc3c2)C1]

A mixture of 6-(azetidin-3-ylmethoxy)-1-chloroisoquinoline hydrochloride (0.400 g, 1.40 mmol) and acetyl chloride (0.220 g, 2.81 mmol) in pyridine (1 mL) was stirred at ambient temperature for 12 h. The reaction mixture was poured into water (30 mL) and extracted with ethyl acetate (3×30 mL). The combined organic phase was washed with brine (30 mL), dried over anhydrous sodium sulfate, and filtered. The filtrate was concentrated in vacuo and the residue was purified by silica gel column chromatography, eluting with 30% of methanol in ethyl acetate: methanol=2:1). The desired fraction was collected and concentrated in vacuo. To the obtained residue was added water (30 mL) and the pH of resulting mixture was adjusted to 7 with 1 N hydrochloric acid. The mixture was extracted with ethyl acetate (3×30 mL). The combined organic phase washed with brine (30 mL), dried over anhydrous sodium sulfate, and filtered. Concentration of the filtrate in vacuo gave the title compounds as a yellow oil (0.200 g, 39% yield): ¹H NMR (400 MHz, DMSO-d₆) δ 8.21 (d, J=5.6 Hz, 1H), 8.17 (d, J=9.2 Hz, 1H), 7.76 (d, J=5.6 Hz, 1H), 7.53 (d, J=2.4 Hz, 1H), 7.43 (dd, J=9.2, 2.4 Hz, 1H), 4.37-4.30 (m, 2H), 4.26 (t, J=8.4 Hz, 1H), 4.01-3.95 (m, 2H), 3.69 (dd, J=9.6, 5.6 Hz, 1H), 3.08 (t, J=6.2 Hz, 1H), 1.76 (s, 3H); MS (ES+) m/z 291.1 (M+1), 293.1 (M+1).

### Step 4. Preparation of 1-(3-(((1-((6-chloropyridin-3-yl)amino)isoquinolin-6-yl)oxy)methyl)azetidin-1-yl)ethan-1-one

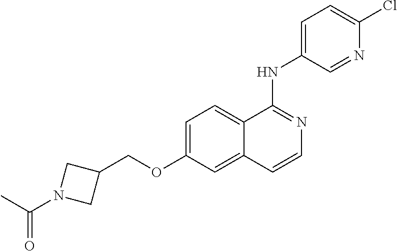 [SMILES: CC(=O)N1CC(COc2ccc3c(Nc4ccc(Cl)nc4)nccc3c2)C1]

A mixture of 1-(3-(((1-chloroisoquinolin-6-yl)oxy)methyl)azetidin-1-yl)ethan-1-one (0.170 g, 0.585 mmol), 6-chloropyridin-3-amine (0.075 g, 0.585 mmol), methanesulfonato(2-di-t-butylphosphino-2′,4′,6′-tri-i-propyl-1,1′-biphenyl)(2′-amino-1,1-biphenyl-2-yl)palladium(II) (0.047 g, 0.059 mmol), and cesium carbonate (0.381 g, 1.17 mmol) in 2-methylbutan-2-ol (1 mL) was stirred at 70° C. for 12 h. After cooling to ambient temperature, the reaction mixture was poured into water (30 mL) and extracted with ethyl acetate (3×30 mL). The combined organic phase was washed with brine (30 mL), dried over anhydrous sodium sulfate, and filtered. The filtrate was concentrated in vacuo and the obtained residue was purified by reverse-phase preparative HPLC (Phenomenex Luna C18 150 mm×25 mm, 10 μm column), eluting with a gradient of 5 to 35% of acetonitrile in water (containing 0.225% of formic acid). The obtained residue was then purified by reverse-phase preparative HPLC (Phenomenex Gemini-NX C18 75 mm×30 mm, 3 μm column), eluting with a gradient of 22 to 52% of acetonitrile in water (containing 10 mM ammonium bicarbonate, to give the title compound as an off-white solid (0.007 g, 3% yield): ¹H NMR (400 MHz, CDCl₃) δ 8.54 (d, J=2.8 Hz, 1H), 8.35 (dd, J=8.8, 2.8 Hz, 1H), 8.03-7.97 (m, 2H), 7.76-7.45 (m, 1H), 7.31 (d, J=8.8 Hz, 1H), 7.21 (dd, J=9.2, 2.4 Hz, 1H), 7.11 (d, J=5.8 Hz, 1H), 7.07 (d, J=2.4 Hz, 1H), 4.34 (t, J=8.4 Hz, 1H), 4.30-4.16 (m, 3H), 4.09 (dd, J=8.4, 5.4 Hz, 1H), 3.95 (dd, J=9.8, 5.4 Hz, 1H), 3.19-3.08 (m, 1H), 1.91 (s, 3H); MS (ES+) m/z 383.1 (M+1), 385.1 (M+1).

### Example 310

### Synthesis of N-(6-chloropyridin-3-yl)-6-((1-(2,2,2-trifluoroethyl)azetidin-3-yl)methoxy)isoquinolin-1-amine

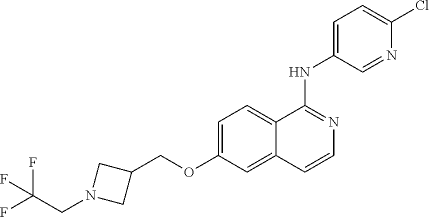 [SMILES: FC(F)(F)CN1CC(COc2ccc3c(Nc4ccc(Cl)nc4)nccc3c2)C1]

### Step 1. Preparation of 1-chloro-6-((1-(2,2,2-trifluoroethyl)azetidin-3-yl)methoxy)isoquinoline

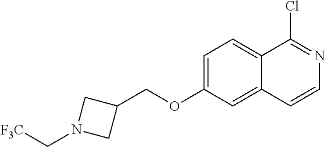 [SMILES: FC(F)(F)CN1CC(COc2ccc3c(Cl)nccc3c2)C1]

A mixture of 6-(azetidin-3-ylmethoxy)-1-chloroisoquinoline hydrochloride (0.400 g, 1.40 mmol), 2,2,2-trifluoroethyl trifluoromethanesulfonate (0.391 g, 1.68 mmol), and triethylamine (0.710 g, 7.01 mmol) in tetrahydrofuran (1 mL) was stirred at 70° C. for 12 h. After cooling to ambient temperature, the reaction mixture was poured into water (30 mL) and extracted with ethyl acetate (3×30 mL). The combined organic phase was washed with brine (30 mL), dried over anhydrous sodium sulfate, and filtered. The filtrate was concentrated in vacuo and the obtained residue was purified by silica gel column chromatography, eluting with a 50% of ethyl acetate in petroleum the, to give the title compound as an off-white solid (0.180 g, 38% yield): ¹H NMR (400 MHz, DMSO-d₆) δ 8.20 (d, J=5.6 Hz, 1H), 8.16 (d, J=9.2 Hz, 1H), 7.75 (d, J=5.6 Hz, 1H), 7.52 (d, J=2.4 Hz, 1H), 7.42 (dd, J=9.2, 2.4 Hz, 1H), 4.30 (d, J=6.8 Hz, 2H), 3.54 (t, J=7.6 Hz, 2H), 3.27-3.17 (m, 4H), 2.92-2.97 (m, 1H); MS (ES+) m/z 331.0 (M+1), 333.0 (M+1).

### Step 2. Preparation of N-(6-chloropyridin-3-yl)-6-((1-(2,2,2-trifluoroethyl)azetidin-3-yl)methoxy)isoquinolin-1-amine

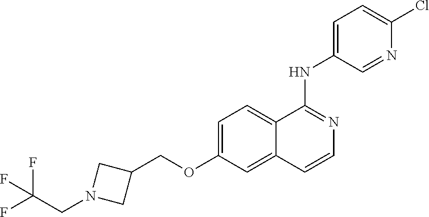 [SMILES: FC(F)(F)CN1CC(COc2ccc3c(Nc4ccc(Cl)nc4)nccc3c2)C1]

Following the procedure as described for EXAMPLE 309, Step 4, and making variations as required to replace 1-(3-(((1-chloroisoquinolin-6-yl)oxy)methyl)azetidin-1-yl)ethan-1-one with 1-chloro-6-((1-(2,2,2-trifluoroethyl)azetidin-3-yl)methoxy)isoquinoline, the title compound was obtained as a colorless solid (0.018 g, 8% yield): ¹H NMR (400 MHz, CDCl₃) δ8.51 (d, J=2.8 Hz, 1H), 8.34 (dd, J=8.8, 2.8 Hz, 1H), 8.01 (d, J=5.8 Hz, 1H), 7.88 (d, J=9.2 Hz, 1H), 7.32 (d, J=8.8 Hz, 1H), 7.21 (dd, J=9.2, 2.4 Hz, 1H), 7.12 (d, J=5.8 Hz, 1H), 7.08 (d, J=2.4 Hz, 1H), 4.34-4.26 (m, 2H), 3.65 (t, J=7.6 Hz, 2H), 3.37 (t, J=6.6 Hz, 2H), 3.12-3.00 (m, 3H), NH not observed; ¹⁹F NMR (376 MHz, DMSO-d₆) δ−71.0 (s); MS (ES+) m/z 423.1 (M+1), 425.1 (M+1).

### Example 311

### Synthesis of N-(6-chloropyridin-3-yl)-6-((1-methyl-1H-pyrazol-4-yl)oxy)isoquinolin-1-amine

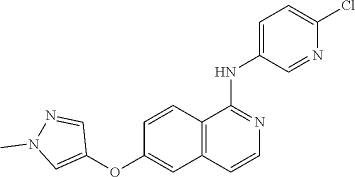 [SMILES: Cn1cc(Oc2ccc3c(Nc4ccc(Cl)nc4)nccc3c2)cn1]

To a solution of 6-((1H-pyrazol-4-yl)oxy)-N-(6-chloropyridin-3-yl)isoquinolin-1-amine (0.051 g, 0.109 mmol) in N,N-dimethylformamide (2 mL) was added sodium hydride (60% dispersion in mineral oil, 0.009 g, 0.218 mmol) and iodomethane (0.010 mL, 0.163 mmol). The reaction mixture was stirred at ambient temperature for 1 h, and then concentrated in vacuo. The obtained residue was dissolved in dichloromethane (2 mL), and to it was added trifluoroacetic acid (1 mL, 7.4 mmol) at ambient temperature. The mixture was stirred at ambient temperature for 2 h, and then concentrated in vacuo. The obtained residue was purified by reverse-phase preparative HPLC, eluting with a gradient of 15 to 60% of acetonitrile in water (containing 0.5% of formic acid), to provide the title compound as a colorless solid (0.014 g, 35% yield): ¹H NMR (400 MHz, DMSO-d₆) δ 9.46 (s, 1H), 8.88 (d, J=2.8 Hz, 1H), 8.52 (d, J=9.3 Hz, 1H), 8.42 (dd, J=8.7, 2.8 Hz, 1H), 7.96 (d, J=5.8 Hz, 1H), 7.90 (s, 1H), 7.50-7.42 (m, 3H), 7.23 (d, J=2.6 Hz, 1H), 7.18 (d, J=5.8 Hz, 1H), 3.87 (s, 3H); MS (ES+) m/z 352.0 (M+1), 354.0 (M+1).

### Example 312

### Synthesis of 1-(4-((1-((6-chloropyridin-3-yl)amino)isoquinolin-6-yl)oxy)piperidin-1-yl)ethan-1-one

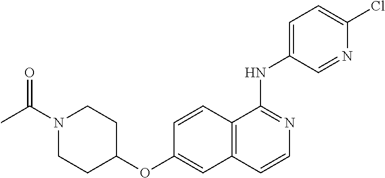 [SMILES: CC(=O)N1CCC(Oc2ccc3c(Nc4ccc(Cl)nc4)nccc3c2)CC1]

### Step 1. Preparation of tert-butyl 4-((1-((6-chloropyridin-3-yl)((2-(trimethylsilyl)ethoxy)methyl)amino)isoquinolin-6-yl)oxy)piperidine-1-carboxylate

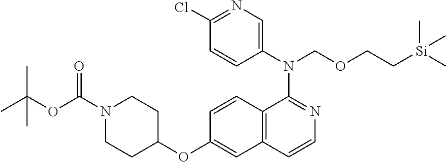 [SMILES: CC(C)(C)OC(=O)N1CCC(Oc2ccc3c(N(COCC[Si](C)(C)C)c4ccc(Cl)nc4)nccc3c2)CC1]

Following the procedure as described for EXAMPLES 264 and 265, Step 1, and making variations as required to replace 4-hydroxycyclohexanone with tert-butyl 4-hydroxy-1-piperidinecarboxylate, the title compound was obtained as a colorless solid (0.288 g, 46% yield): ¹H NMR (400 MHz, CDCl₃) δ 8.32 (d, J=5.7 Hz, 1H), 8.03 (d, J=2.8 Hz, 1H), 7.71 (d, J=9.2 Hz, 1H), 7.45 (d, J=5.8 Hz, 1H), 7.18 (dd, J=8.7, 2.9 Hz, 1H), 7.12 (dd, J=5.5, 3.1 Hz, 2H), 7.08 (dd, J=9.2, 2.5 Hz, 1H), 5.42 (s, 2H), 4.71-4.65 (m, 1H), 3.76-3.70 (m, 2H), 3.56 (t, J=8.2 Hz, 2H), 3.46-3.40 (m, 2H), 2.05-1.97 (m, 2H), 1.88-1.80 (m, 2H), 1.50 (s, 9H), 0.89 (t, J=8.2 Hz, 2H), −0.09 (s, 9H); MS (ES+) m/z 585.6 (M+1), 587.6 (M+1).

### Step 2. Preparation of 1-(4-((1-((6-chloropyridin-3-yl)amino)isoquinolin-6-yl)oxy)piperidin-1-yl)ethan-1-one

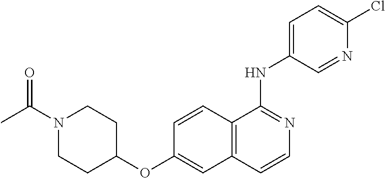 [SMILES: CC(=O)N1CCC(Oc2ccc3c(Nc4ccc(Cl)nc4)nccc3c2)CC1]

To a solution of tert-butyl 4-((1-((6-chloropyridin-3-yl)((2-(trimethylsilyl)ethoxy)methyl)amino)isoquinolin-6-yl)oxy)piperidine-1-carboxylate (0.08 g, 0.136 mmol) in dichloromethane (2 mL) was added trifluoroacetic acid (1 mL, 13.1 mmol) at ambient temperature. The mixture was stirred at ambient temperature for 4 h, and then concentrated in vacuo to provide a residue. The residue was dissolved in dichloromethane (2 mL), and to it was added triethylamine (0.11 mL, 0.817 mmol) and acetyl chloride (0.012 mL, 0.163 mmol). The mixture was stirred at ambient temperature for 1 h, and then concentrated in vacuo. The obtained residue was purified by reverse-phase preparative HPLC, eluting with a gradient of 15 to 80% of acetonitrile in water (containing 0.5% of formic acid), to provide the title compound as a colorless solid (0.010 g, 18% yield): ¹H NMR (400 MHz, DMSO-d₆) δ 9.41-9.37 (m, 1H), 8.91-8.88 (m, 1H), 8.49-8.39 (m, 2H), 7.99-7.96 (m, 1H), 7.51-7.44 (m, 1H), 7.41-7.38 (m, 1H), 7.33-7.28 (m, 1H), 7.21-7.17 (m, 1H), 4.87-4.83 (m, 1H), 3.95-3.89 (m, 1H), 3.76-3.70 (m, 1H), 3.41-3.36 (m, 1H), 3.30-3.23 (m, 1H), 2.10-1.96 (m, 5H), 1.73-1.53 (m, 2H); MS (ES+) m/z 397.2 (M+1), 399.2 (M+1).

### Example 313

### Synthesis of N-(6-chloropyridin-3-yl)-6-((1-(oxetan-3-yl)piperidin-4-yl)oxy)isoquinolin-1-amine

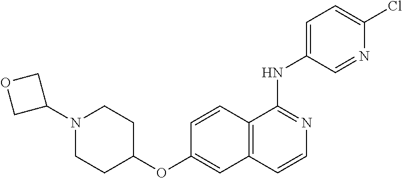 [SMILES: Clc1ccc(Nc2nccc3cc(OC4CCN(C5COC5)CC4)ccc23)cn1]

To a solution of tert-butyl 4-((1-((6-chloropyridin-3-yl)((2-(trimethylsilyl)ethoxy)methyl)amino)isoquinolin-6-yl)oxy)piperidine-1-carboxylate (0.096 g, 0.164 mmol) in dichloromethane (2 mL) was added trifluoroacetic acid (1 mL, 13.1 mmol) at ambient temperature. The mixture was stirred at ambient temperature for 1 h, and then concentrated in vacuo to provide a residue. The residue was dissolved in dichloromethane (2 mL), and to it was added 3-oxetanone (0.35 g, 0.492 mmol). The mixture was stirred at ambient temperature for 10 minutes, followed by addition of sodium triacetoxyborohydride (0.104 g, 0.492 mmol). The reaction mixture was stirred at ambient temperature for 16 h. The mixture was then diluted with ethyl acetate (20 mL), and washed with saturated aqueous sodium bicarbonate (20 mL) and brine (20 mL). The organic phase was dried over anhydrous sodium sulfate and filtered. The filtrate was concentrated in vacuo to give a residue. The residue was purified by silica gel column chromatography, eluting with a gradient of 0 to 100% of ethyl acetate in heptane, to provide the title compound as a colourless solid (0.012 g, 18% yield): ¹H NMR (400 MHz, DMSO-d₆) δ9.37 (s, 1H), 8.88 (d, J=2.8 Hz, 1H), 8.44-8.41 (m, 2H), 7.95 (d, J=5.8 Hz, 1H), 7.45 (d, J=8.8 Hz, 1H), 7.34 (d, J=2.5 Hz, 1H), 7.28 (dd, J=9.2, 2.6 Hz, 1H), 7.17 (d, J=5.9 Hz, 1H), 4.66-4.62 (m, 1H), 4.57-4.53 (m, 2H), 4.46-4.42 (m, 2H), 3.47-3.40 (m, 1H), 2.59-2.53 (m, 2H), 2.18-2.12 (m, 2H), 2.08-2.02 (m, 2H), 1.76-1.68 (m, 2H); MS (ES+) m/z 411.0 (M+1), 413.0 (M+1).

### Example 314

### Synthesis of N-(6-chloropyridin-3-yl)-6-((1-(pyrimidin-2-ylmethyl)piperidin-4-yl)oxy)isoquinolin-1-amine

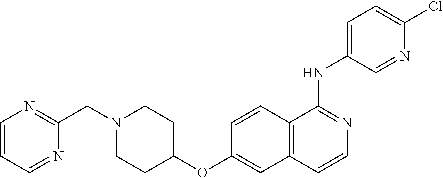 [SMILES: Clc1ccc(Nc2nccc3cc(OC4CCN(Cc5ncccn5)CC4)ccc23)cn1]

Following the procedure as described for EXAMPLE 313, and making variations as required to replace 3-oxetanone with 2-pyrimidinecarboxaldehyde, the title compound was obtained as a colorless solid (0.024 g, 33% yield): ¹H NMR (400 MHz, DMSO-d₆) δ9.37 (s, 1H), 8.88 (d, J=2.8 Hz, 1H), 8.80 (d, J=4.9 Hz, 2H), 8.44-8.41 (m, 2H), 7.94 (d, J=5.8 Hz, 1H), 7.43 (dd, J=11.5, 6.7 Hz, 2H), 7.33 (d, J=2.6 Hz, 1H), 7.27 (dd, J=9.2, 2.5 Hz, 1H), 7.17 (d, J=5.8 Hz, 1H), 4.63-4.55 (m, 1H), 3.77 (d, J=0.2 Hz, 2H), 2.87-2.82 (m, 2H), 2.48-2.43 (m, 2H), 2.07-2.01 (m, 2H), 1.74-1.65 (m, 2H); MS (ES+) m/z 447.0 (M+1), 449.0 (M+1).

### Example 315

### Synthesis of N-(6-chloropyridin-3-yl)-6-((1-(2,2-difluoroethyl)piperidin-4-yl)oxy)isoquinolin-1-amine

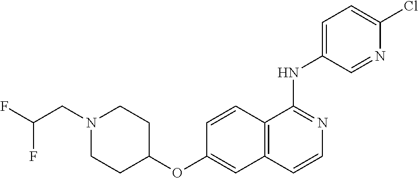 [SMILES: FC(F)CN1CCC(Oc2ccc3c(Nc4ccc(Cl)nc4)nccc3c2)CC1]

Following the procedure as described for EXAMPLE 119, and making variations as required to replace 1-bromo-2-methoxyethane with 2-iodo-1,1-difluoroethane, the title compound was obtained as a colorless solid (0.040 g, 55% yield): ¹H NMR (400 MHz, DMSO-d₆) δ9.37 (s, 1H), 8.88 (d, J=2.8 Hz, 1H), 8.44-8.41 (m, 2H), 7.95 (d, J=5.8 Hz, 1H), 7.45 (d, J=8.8 Hz, 1H), 7.34 (d, J=2.5 Hz, 1H), 7.28 (dd, J=9.2, 2.6 Hz, 1H), 7.18 (d, J=5.7 Hz, 1H), 6.31-6.01 (m, 1H), 4.65-4.59 (m, 1H), 2.87-2.73 (m, 4H), 2.48-2.47 (m, 2H), 2.05-1.99 (m, 2H), 1.75-1.66 (m, 2H); MS (ES+) m/z 419.2 (M+1), 421.2 (M+1).

### Examples 316 and 317

### Synthesis of N-(cis-4-((1-((6-chloropyridin-3-yl)amino)isoquinolin-6-yl)oxy)cyclohexyl)acetamide and N-(trans-4-((1-((6-chloropyridin-3-yl)amino)isoquinolin-6-yl)oxy)cyclohexyl)acetamide

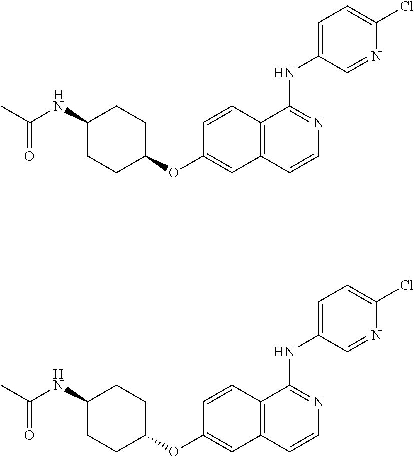 [SMILES: CC(=O)N[C@H]1CC[C@@H](Oc2ccc3c(Nc4ccc(Cl)nc4)nccc3c2)CC1 ; CC(=O)N[C@H]1CC[C@H](Oc2ccc3c(Nc4ccc(Cl)nc4)nccc3c2)CC1]

### Step 1. Preparation of 6-((4-aminocyclohexyl)oxy)-N-(6-chloropyridin-3-yl)-N-((2-(trimethylsilyl)ethoxy)methyl)isoquinolin-1-amine

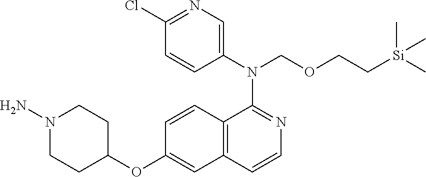 [SMILES: C[Si](C)(C)CCOCN(c1ccc(Cl)nc1)c1nccc2cc(OC3CCN(N)CC3)ccc12]

To a solution of 4-((1-((6-chloropyridin-3-yl)((2-(trimethylsilyl)ethoxy)methyl)amino)isoquinolin-6-yl)oxy)cyclohexan-1-one (0.085 g, 0.171 mmol) in dichloromethane (1 mL) and methanol (1 mL) was added ammonium acetate (0.15 mL, 1.71 mmol). The mixture was stirred at ambient temperature for 1 h and then to it was then added sodium cyanoborohydride (0.021 g, 0.341 mmol). The reaction mixture was stirred at ambient temperature for 30 minutes. The mixture was then diluted with ethyl acetate (30 mL) and washed with saturated sodium bicarbonate (30 mL) and brine (30 mL). The organic phase was dried over anhydrous sodium sulfate and filtered. The filtrate was concentrated in vacuo to give the title compound as colorless solid (0.075 g, 31% yield): MS (ES+) m/z 499.2 (M+1), 501.2 (M+1).

### Step 2. Preparation of N-(cis-4-((1-((6-chloropyridin-3-yl)amino)isoquinolin-6-yl)oxy)cyclohexyl)acetamide and N-(trans-4-((1-((6-chloropyridin-3-yl)amino)isoquinolin-6-yl)oxy)cyclohexyl)acetamide

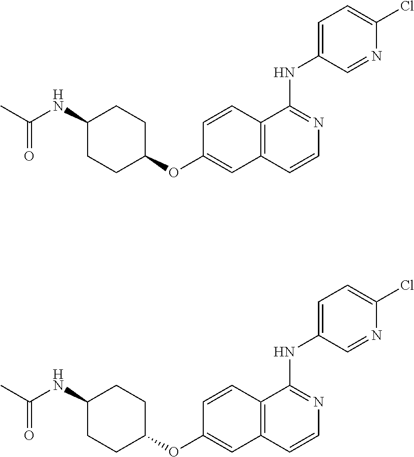 [SMILES: CC(=O)N[C@H]1CC[C@@H](Oc2ccc3c(Nc4ccc(Cl)nc4)nccc3c2)CC1 ; CC(=O)N[C@H]1CC[C@H](Oc2ccc3c(Nc4ccc(Cl)nc4)nccc3c2)CC1]

Following the procedure as described for EXAMPLE 312, Step 2, and making variations as required to replace tert-butyl 4-((1-((6-chloropyridin-3-yl)((2-(trimethylsilyl)ethoxy)methyl)amino)isoquinolin-6-yl)oxy)piperidine-1-carboxylate with 6-((4-aminocyclohexyl)oxy)-N-(6-chloropyridin-3-yl)-N-((2-(trimethylsilyl)ethoxy)methyl)isoquinolin-1-amine, a mixture of the title compounds was obtained. Separation of the mixture by chiral SFC (ChiralPak AS, 10×250 mm, 5 μm column), eluting with 40% of methanol (containing 10 mM ammonium formate) in supercritical carbon dioxide, afforded the title compounds as single diastereoisomers as colorless solids. First eluting diastereomer (0.009 g, 13% yield): ¹H NMR (400 MHz, DMSO-d₆) δ 9.37 (s, 1H), 8.88 (d, J=2.7 Hz, 1H), 8.42 (dd, J=8.8, 2.8 Hz, 2H), 7.95 (d, J=5.8 Hz, 1H), 7.85-7.83 (m, 1H), 7.45 (d, J=8.8 Hz, 1H), 7.35 (d, J=2.5 Hz, 1H), 7.25 (dd, J=9.2, 2.5 Hz, 1H), 7.19 (d, J=5.7 Hz, 1H), 4.57-4.50 (m, 1H), 3.63-3.57 (m, 1H), 2.17-2.13 (m, 2H), 1.89-1.85 (m, 2H), 1.81 (s, 3H), 1.55-1.34 (m, 4H); MS (ES+) m/z 411.2 (M+1), 413.2 (M+1). Second eluting diastereomer (0.012 g, 19% yield): ¹H NMR (400 MHz, DMSO-d₆) d 9.38 (s, 1H), 8.88 (d, J=2.8 Hz, 1H), 8.43 (dt, J=8.6, 4.3 Hz, 2H), 7.95 (d, J=5.8 Hz, 1H), 7.85 (d, J=7.5 Hz, 1H), 7.45 (d, J=8.8 Hz, 1H), 7.31 (d, J=2.4 Hz, 1H), 7.26 (dd, J=9.2, 2.5 Hz, 1H), 7.17 (d, J=5.8 Hz, 1H), 4.75-4.72 (m, 1H), 3.75-3.68 (m, 1H), 1.99-1.93 (m, 2H), 1.81-1.79 (m, 3H), 1.79-1.71 (m, 2H), 1.67-1.56 (m, 4H); MS (ES+) m/z 411.2 (M+1), 413.2 (M+1).

### Example 318

### Synthesis of 1-(4-(((1-((6-chloropyridin-3-yl)amino)isoquinolin-6-yl)oxy)methyl)piperidin-1-yl)ethan-1-one

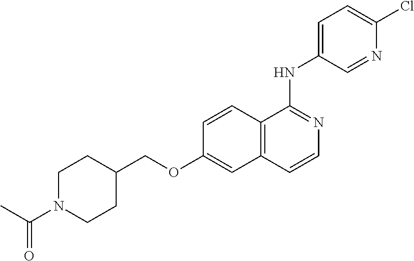 [SMILES: CC(=O)N1CCC(COc2ccc3c(Nc4ccc(Cl)nc4)nccc3c2)CC1]

### Step 1. Preparation of tert-butyl 4-(((1-((6-chloropyridin-3-yl)((2-(trimethylsilyl)ethoxy)methyl)amino)isoquinolin-6-yl)oxy)methyl)piperidine-1-carboxylate

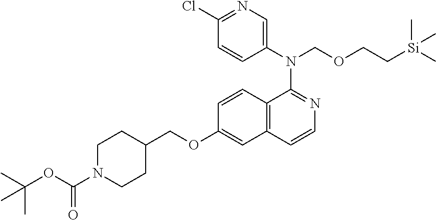 [SMILES: CC(C)(C)OC(=O)N1CCC(COc2ccc3c(N(COCC[Si](C)(C)C)c4ccc(Cl)nc4)nccc3c2)CC1]

Following the procedure as described for EXAMPLE 264 and 265, Step 1, and making variations as required to replace 4-hydroxycyclohexanone with tert-butyl 4-(hydroxymethyl)piperidine-1-carboxylate, the title compound was obtained as a colorless solid (0.418 g, 82% yield): ¹H NMR (400 MHz, CDCl₃) δ 8.32 (d, J=5.7 Hz, 1H), 8.03 (dd, J=3.0, 0.3 Hz, 1H), 7.70-7.68 (m, 1H), 7.47-7.45 (m, 1H), 7.17-7.12 (m, 2H), 7.10-7.06 (m, 2H), 5.42 (s, 2H), 4.24-4.12 (m, 2H), 3.95 (d, J=6.2 Hz, 2H), 3.56 (dd, J=8.7, 7.7 Hz, 2H), 2.84-2.73 (m, 2H), 2.08-2.02 (m, 1H), 1.90-1.85 (m, 2H), 1.49 (s, 9H), 1.30-1.26 (m, 2H), 0.89 (t, J=8.2 Hz, 2H), −0.09 (s, 9H); MS (ES+) m/z 599.5 (M+1), 601.5 (M+1).

### Step 2. Preparation of 1-(4-(((1-((6-chloropyridin-3-yl)amino)isoquinolin-6-yl)oxy)methyl)piperidin-1-yl)ethan-1-one

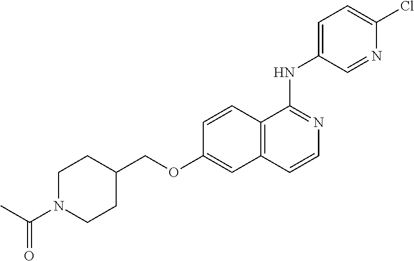 [SMILES: CC(=O)N1CCC(COc2ccc3c(Nc4ccc(Cl)nc4)nccc3c2)CC1]

Following the procedure as described for EXAMPLE 312, Step 2, and making variations as required to replace tert-butyl 4-((1-((6-chloropyridin-3-yl)((2-(trimethylsilyl)ethoxy)methyl)amino)isoquinolin-6-yl)oxy)piperidine-1-carboxylate with tert-butyl 4-(hydroxymethyl)piperidine-1-carboxylate with tert-butyl 4-(((1-((6-chloropyridin-3-yl)((2-(trimethylsilyl)ethoxy)methyl)amino)isoquinolin-6-yl)oxy)methyl)piperidine-1-carboxylate, the title compound was obtained as a colorless solid (0.028 g, 39% yield): ¹H NMR (400 MHz, DMSO-d₆) δ9.38 (s, 1H), 8.88 (d, J=2.8 Hz, 1H), 8.45-8.42 (m, 2H), 7.96 (d, J=5.8 Hz, 1H), 7.45 (d, J=8.8 Hz, 1H), 7.28 (dd, J=7.8, 2.3 Hz, 2H), 7.19 (d, J=5.7 Hz, 1H), 4.45-4.41 (m, 1H), 4.01 (d, J=6.3 Hz, 2H), 3.89-3.85 (m, 1H), 3.11-3.04 (m, 1H), 2.61-2.53 (m, 1H), 2.12-2.04 (m, 1H), 2.05-2.01 (m, 3H), 1.88-1.79 (m, 2H), 1.35-1.11 (m, 2H); MS (ES+) m/z 411.2 (M+1), 413.2 (M+1).

### Examples 319

### Synthesis of N-(6-chloropyridin-3-yl)-6-(2-((2S,6R)-2,6-dimethylmorpholino)ethoxy)isoquinolin-1-amine

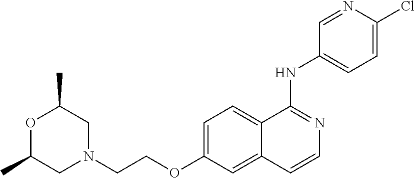 [SMILES: C[C@@H]1CN(CCOc2ccc3c(Nc4ccc(Cl)nc4)nccc3c2)C[C@H](C)O1]

To a solution of 6-(2-bromoethoxy)-N-(6-chloropyridin-3-yl)isoquinolin-1-amine (0.050 g, 0.132 mmol) in acetonitrile (1 mL) was added (2R,6S)-2,6-dimethylmorpholine (0.033 mL, 0.264 mmol) and potassium carbonate (0.036 g, 0.264 mmol). The reaction mixture was stirred at 80° C. for 5 h. After cooling to ambient temperature, the reaction mixture was filtered, and the filtrate was concentrated in vacuo. The obtained residue was purified by reverse-phase preparative HPLC, eluting with a gradient of 10 to 60% of acetonitrile in water (containing 0.5% of formic acid), to provide the title compound as a colorless solid (0.050 g, 89% yield): ¹H NMR (400 MHz, CDCl₃) δ 9.38 (s, 1H), 8.89-8.88 (m, 1H), 8.43 (dd, J=8.8, 2.9 Hz, 2H), 7.97 (d, J=5.7 Hz, 1H), 7.45 (d, J=8.6 Hz, 1H), 7.33-7.27 (m, 2H), 7.18 (d, J=5.8 Hz, 1H), 4.24 (t, J=5.8 Hz, 2H), 3.60-3.54 (m, 2H), 2.88-2.85 (m, 2H), 2.75 (dd, J=6.7, 5.0 Hz, 2H), 1.75 (dd, J=11.0, 10.4 Hz, 2H), 1.06 (d, J=6.3 Hz, 6H); MS (ES+) m/z 413.2 (M+1), 415.2 (M+1).

### Examples 320-323

In a similar manner as described in EXAMPLE 319, utilizing the appropriately substituted starting materials and intermediates, the following compounds were prepared:

| Example No. | Name | MS (ES+) m/z |
|---|---|---|
| 320 | methyl 4-(2-((1-((6-chloropyridin-3- | 443.2 (M + 1), |
| | yl)amino)isoquinolin-6-yl)oxy)ethyl)morpholine- | 445.2 (M + 1). |
| | 3-carboxylate | |
| 321 | (S)-N-(6-chloropyridin-3-yl)-6-(2-(3- | 399.2 (M + 1), |
| | methylmorpholino) ethoxy)isoquinolin-1-amine | 401.2 (M + 1). |
| 322 | (R)-N-(6-chloropyridin-3-yl)-6-(2-(3- | 399.2 (M + 1), |
| | methylmorpholino)ethoxy)isoquinolin-1-amine | 401.2 (M + 1). |
| 323 | N-(6-chloropyridin-3-yl)-6-(2-(2- | 399.2 (M + 1), |
| | methylmorpholino)ethoxy)isoquinolin-1-amine | 401.2 (M + 1). |

### Example 324

### Synthesis of 2-methyl-5-((6-((1-methyl-1H-pyrazol-4-yl)methoxy)isoquinolin-1-yl)amino)pyridin-3-ol

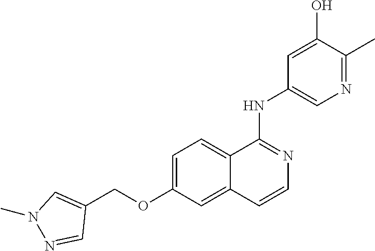

### Step 1. Preparation of 6-((1-methyl-1H-pyrazol-4-yl)methoxy)isoquinolin-1-amine

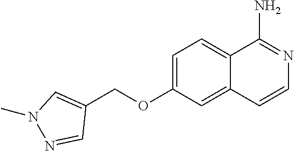 [SMILES: Cn1cc(COc2ccc3c(N)nccc3c2)cn1]

To a mixture of 1-chloro-6-((1-methyl-1H-pyrazol-4-yl)methoxy)isoquinoline (0.300 g, 1.10 mmol) and tert-butyl carbamate (0.257 g, 2.19 mmol) in 1,4-dioxane (10 mL) was added potassium tert-butoxide (0.369 g, 3.29 mmol) and [(2-di-tert-butylphosphino-2′,4′,6′-triisopropyl-1,1′-biphenyl)-2-(2′-amino-1,1′-biphenyl)] palladium(II) methanesulfonate (0.087 g, 0.110 mmol). The reaction mixture was stirred at 90° C. for 18 h. After cooling to ambient temperature, the reaction mixture was poured into water (10 mL). The mixture was extracted with ethyl acetate (3×10 mL). The combined organic layers were washed with brine (10 mL), dried over anhydrous sodium sulfate, and filtered. The filtrate was concentrated in vacuo and the obtained residue was purified by normal-phase preparative HPLC (Welch Ultimate XB SiO₂ 100 mm×30 mm, 10 μm column), eluting with a gradient of 20% to 60% of ethanol in n-hexane containing 0.1% of ammonium hydroxide, to provide the title compound as a brownish solid (0.100 g, 36% yield): MS (ES+) m/z 255.2 (M+1).

### Step 2. Preparation of 2-methyl-5-((6-((1-methyl-1H-pyrazol-4-yl)methoxy)isoquinolin-1-yl)amino)pyridin-3-ol

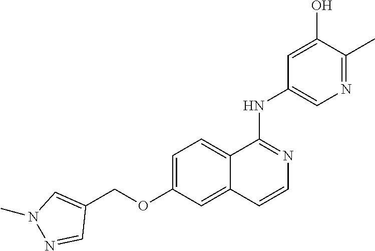

To a mixture of 6-((1-methyl-1H-pyrazol-4-yl)methoxy)isoquinolin-1-amine (0.080 g, 0.315 mmol), 5-bromo-2-methylpyridin-3-ol (0.071 g, 0.378 mmol) in 2-methylbutan-2-ol (8 mL) was added potassium tert-butoxide (0.106 g, 0.943 mmol) and [(2-di-tert-butylphosphino-2′,4′,6′-triisopropyl-1,1′-biphenyl)-2-(2′-amino-1,1′-biphenyl)] palladium(II) methanesulfonate (0.050 g, 0.063 mmol). The reaction mixture was stirred at 100° C. for 20 h. After cooling to ambient temperature, the reaction mixture was poured into water (10 mL). The mixture was extracted with ethyl acetate (3×10 mL). The combined organic layers were washed with brine (10 mL), dried over anhydrous sodium sulfate, and filtered. The filtrate was concentrated in vacuo and the obtained residue was purified by reverse-phase preparative HPLC (Waters XBridge 150 mm×25 mm, 5 μm column), eluting with a gradient of 20% to 50% of acetonitrile in water (containing 10 mM of ammonium bicarbonate), to provide the title compound as a yellow solid (0.009 g, 7% yield): ¹H NMR (400 MHz, DMSO-d₆) δ 9.57 (s, 1H), 9.01 (s, 1H), 8.43 (d, J=9.3 Hz, 1H), 8.29 (d, J=2.1 Hz, 1H), 7.90 (dd, J=8.0, 3.9 Hz, 2H), 7.85 (s, 1H), 7.56 (s, 1H), 7.35 (d, J=2.5 Hz, 1H), 7.21 (dd, J=9.2, 2.5 Hz, 1H), 7.10 (d, J=5.8 Hz, 1H), 5.10 (s, 2H), 3.83 (s, 3H), 2.28 (s, 3H); MS (ES+) m/z 362.2 (M+1).

### Example 325

### Synthesis of N-(6-chloropyridin-3-yl)-6-((1-(2,2,2-trifluoroethyl)azetidin-3-yl)oxy)isoquinolin-1-amine

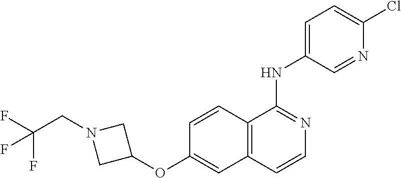 [SMILES: FC(F)(F)CN1CC(Oc2ccc3c(Nc4ccc(Cl)nc4)nccc3c2)C1]

### Step 1. Preparation of tert-butyl 3-((1-chloroisoquinolin-6-yl)oxy)azetidine-1-carboxylate

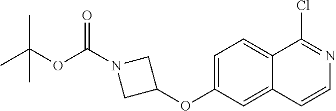 [SMILES: CC(C)(C)OC(=O)N1CC(Oc2ccc3c(Cl)nccc3c2)C1]

To a solution of tert-butyl 3-iodoazetidine-1-carboxylate (0.950 g, 3.36 mmol) in N,N-dimethylformamide (5 mL) was added potassium carbonate (1.15 g, 8.35 mmol) and 1-chloroisoquinolin-6-ol (0.500 g, 2.78 mmol) at ambient temperature. The reaction mixture was heated up to 90° C. for 2 h. After cooling to ambient temperature, the reaction mixture was diluted with ethyl acetate (20 mL). The organic phase was washed with water (3×20 mL), dried over anhydrous sodium sulfate, and filtered. The filtrate was concentrated in vacuo and the obtained residue was purified by silica gel column chromatography, eluting with a gradient of 0 to 34% of ethyl acetate in petroleum ether, to provide the title compound as a yellow oil (0.922 g, 98% yield): MS (ES+) m/z 335.2 (M+1), 337.2 (M+1).

### Step 2. Preparation of 6-(azetidin-3-yloxy)-1-chloroisoquinoline hydrochloride

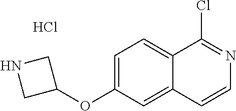 [SMILES: Clc1nccc2cc(OC3CNC3)ccc12 ; Cl]

To a solution of tert-butyl 3-((1-chloroisoquinolin-6-yl)oxy)azetidine-1-carboxylate (0.922 g, 2.75 mmol) in dichloromethane (5 mL) was added a 4 M solution of hydrogen chloride in dioxane (5 mL, 20.0 mmol). The mixture was stirred at ambient temperature for 1 h and then concentrated in vacuo to give the title compound as a colorless solid (0.740 g, 94% yield): ¹H NMR (400 MHz, DMSO-d₆) δ9.57-9.37 (m, 2H), 8.29-8.19 (m, 2H), 7.79 (d, J=5.6 Hz, 1H), 7.46 (dd, J=9.2, 2.4 Hz, 1H), 7.37 (d, J=2.4 Hz, 1H), 5.31-5.23 (m, 1H), 4.53 (dd, J=12.0, 6.0 Hz, 2H), 4.08-4.04 (m, 2H); MS (ES+) m/z 235.2 (M+1), 237.2 (M+1).

### Step 3. Preparation of 1-chloro-6-((1-(2,2,2-trifluoroethyl)azetidin-3-yl)oxy)isoquinoline

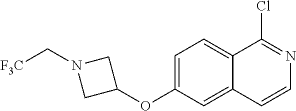 [SMILES: FC(F)(F)CN1CC(Oc2ccc3c(Cl)nccc3c2)C1]

To a solution of 6-(azetidin-3-yloxy)-1-chloroisoquinoline hydrochloride (0.150 g, 0.553 mmol) and triethylamine (0.285 g, 2.82 mmol) in tetrahydrofuran (2 mL) was added 2,2,2-trifluoroethyl trifluoromethanesulfonate (0.154 g, 0.664 mmol) at ambient temperature. The mixture was then stirred for 12 h at 75° C. After cooling to ambient temperature, the mixture was concentrated in vacuo. The residue was purified by silica gel column chromatography, eluting with a gradient of 0 to 25% of ethyl acetate in petroleum ether, to provide the title compound as a colorless oil (0.120 g, 66% yield): ¹H NMR (400 MHz, DMSO-d₆) δ8.24-8.15 (m, 2H), 7.78 (d, J=5.6 Hz, 1H), 7.41 (dd, J=9.2, 2.4 Hz, 1H), 7.32 (d, J=2.4 Hz, 1H), 5.05 (t, J=5.6 Hz, 1H), 4.00 (dd, J=8.4, 6.4 Hz, 2H), 3.34 (s, 4H); MS (ES+) m/z 317.1 (M+1), 319.1 (M+1).

### Step 4. Preparation of N-(6-chloropyridin-3-yl)-6-((1-(2,2,2-trifluoroethyl)azetidin-3-yl)oxy)isoquinolin-1-amine

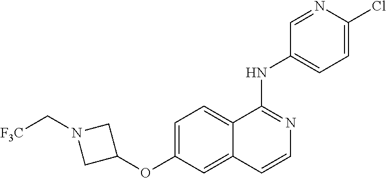 [SMILES: FC(F)(F)CN1CC(Oc2ccc3c(Nc4ccc(Cl)nc4)nccc3c2)C1]

A mixture of 1-chloro-6-((1-(2,2,2-trifluoroethyl)azetidin-3-yl)oxy)isoquinoline (0.110 g, 0.347 mmol), 6-chloropyridin-3-amine (0.045 g, 0.350 mmol), [(2-di-tert-butylphosphino-2′,4′,6′-triisopropyl-1,1′-biphenyl)-2-(2′-amino-1,1′-biphenyl)]palladium(II) methanesulfonate (0.0280 g, 0.0352 mmol), and cesium carbonate (0.340 g, 1.04 mmol) in 2-methylbutan-2-ol (2 mL) was stirred at 90° C. for 12 h. After cooling to ambient temperature, the reaction mixture was poured into water (20 mL). The mixture was extracted with ethyl acetate (3×20 mL). The combined organic layers were washed with brine (10 mL), dried over anhydrous sodium sulfate, and filtered. The filtrate was concentrated in vacuo and the obtained residue was purified by reverse-phase preparative HPLC (Waters XBridge 150 mm×25 mm, 5 μm column), eluting with a gradient of 45% to 75% of acetonitrile in water (containing of 0.05% ammonium hydroxide). The residue was further purified by reverse-phase preparative HPLC (Shim-pack C18 150 mm×25 mm, 10 μm column), eluting with a gradient of 13% to 43% of acetonitrile in water (containing 0.23% of formic acid) to provide the title compound as a colorless solid (0.043 g, 30% yield): ¹H NMR (400 MHz, DMSO-d₆) δ9.39 (m, 1H), 8.90-8.84 (m, 1H), 8.46-8.39 (m, 2H), 7.99-7.93 (m, 1H), 7.44 (dd, J=8.7, 3.3 Hz, 1H), 7.26-7.17 (m, 2H), 7.12-7.09 (m, 1H), 5.04-5.01 (m, 1H), 4.00-3.97 (m, 2H), 3.35-3.27 (m, 4H); ¹⁹F NMR (376 MHz, DMSO-d₆) δ−69.9 (s); MS (ES+) m/z 409.2 (M+1), 411.2 (M+1).

### Example 326

### Synthesis of N-(6-chloropyridin-3-yl)-6-((1-(difluoromethyl)cyclopropyl)methoxy)isoquinolin-1-amine

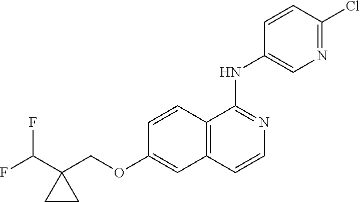 [SMILES: FC(F)C1(COc2ccc3c(Nc4ccc(Cl)nc4)nccc3c2)CC1]

To a solution of (1-(difluoromethyl)cyclopropyl)methanol (0.040 g, 0.290 mmol) in N,N-dimethylformamide (2 mL) was added sodium hydride (60% dispersion in mineral oil, 0.035 g, 0.875 mmol), and the resulting mixture was stirred at ambient temperature for 5 minutes. To the reaction mixture was then added a solution of N-(6-chloropyridin-3-yl)-6-fluoro-N-((2-(trimethylsilyl)ethoxy)methyl)isoquinolin-1-amine (0.100 g, 0.248 mmol) in N,N-dimethylformamide (1 mL) and the mixture was heated to 60° C. for 2 h. After cooling to ambient temperature, the mixture was concentrated in vacuo. To the obtained residue was added dichloromethane (2 mL) and trifluoroacetic acid (1 mL, 13.1 mmol) at ambient temperature. The reaction mixture was stirred at ambient temperature for 2 h, and then concentrated in vacuo. To the residue was added saturated aqueous sodium bicarbonate (20 mL), and the mixture was extracted with ethyl acetate (30 mL). The organic phase was washed with brine (20 mL), dried over anhydrous sodium sulfate, and filtered. The filtrate was concentrated in vacuo to provide a residue, which was purified by silica gel column chromatography, eluting with a gradient of 10 to 100% of ethyl acetate in heptane, to afford the title compound as a colorless solid (0.085 g, 89% yield): ¹H NMR (400 MHz, DMSO-d₆) δ 9.40 (s, 1H), 8.89 (d, J=2.8 Hz, 1H), 8.44 (dt, J=8.8, 4.4 Hz, 2H), 7.97 (d, J=5.8 Hz, 1H), 7.45 (d, J=8.7 Hz, 1H), 7.31 (dd, J=6.1, 2.5 Hz, 2H), 7.17 (d, J=5.8 Hz, 1H), 6.06 (t, J=56.5 Hz, 1H), 4.21 (s, 2H), 0.95-0.87 (m, 4H); MS (ES+) m/z 376.0 (M+1), 378.0 (M+1).

### Examples 327-333

In a similar manner as described in EXAMPLE 326, utilizing the appropriately substituted starting materials and intermediates, the following compounds were prepared:

| | | MS | |
|---|---|---|---|
| Example | | (ES+) | |
| No. | Name | m/z | NMR |
| 327 | N-(6-chloropyridin-3- | 392.0 | ¹H NMR (400 MHZ, DMSO-d₆) δ 9.43 |
| | yl)-6-((3-(difluoro- | (M + 1), | (dt, J = 0.7, 0.4 Hz, 1H), 8.89 (d, J = |
| | methyl)oxetan-3- | 394.0 | 2.8 Hz, 1H), 8.50-8.42 (m, 2H), 7.99 |
| | yl)methoxy)isoquinolin- | (M + 1). | (d, J = 5.8 Hz, 1H), 7.46 (d, J = 8.7 |
| | 1-amine | | Hz, 1H), 7.41 (d, J = 2.5 Hz, 1H), 7.35 |
| | | | (dd, J = 9.3, 2.6 Hz, 1H), 7.21 (d, J = |
| | | | 5.8 Hz, 1H), 6.48 (t, J = 55.8 Hz, 1H), |
| | | | 4.67 (d, J = 6.7 Hz, 2H), 4.62 (d, J = |
| | | | 6.7 Hz, 2H), 4.49 (s, 2H). |
| 328 | N-(6-chloropyridin-3- | 370.2 | ¹H NMR (400 MHZ, DMSO-d₆) δ 9.52 |
| | yl)-6-((3-ethyloxetan-3- | (M + 1), | (s, 1H), 8.88 (d, J = 2.7 Hz, 1H), 8.48 |
| | yl)methoxy)isoquinolin- | 372.2 | (d, J = 9.3 Hz, 1H), 8.42 (dd, J = 8.7, |
| | 1-amine | (M + 1). | 2.6 Hz, 1H), 7.95 (d, J = 5.9 Hz, 1H), |
| | | | 7.48 (d, J = 8.7 Hz, 1H), 7.41 (d, J = |
| | | | 2.4 Hz, 1H), 7.35 (dd, J = 9.2, 2.5 Hz, |
| | | | 1H), 7.21 (d, J = 5.9 Hz, 1H), 4.51 (d, |
| | | | J = 6.0 Hz, 2H), 4.39 (d, J = 6.0 Hz, |
| | | | 2H), 4.28 (s, 2H), 1.85 (q, J = 7.5 Hz, |
| | | | 2H), 0.94 (t, J = 7.5 Hz, 3H). |
| 329 | (3-(((1-((6- | 372.2 | ¹H NMR (400 MHZ, DMSO-d₆) δ 9.40 |
| | chloropyridin-3- | (M + 1), | (s, 1H), 8.89 (d, J = 2.8 Hz, 1H), 8.45 |
| | yl)amino)isoquinolin-6- | 374.0 | (dt, J = 8.9, 4.5 Hz, 2H), 7.98 (d, J = |
| | yl)oxy)methyl)oxetan-3- | (M + 1). | 5.8 Hz, 1H), 7.45 (d, J = 8.7 Hz, 1H), |
| | yl)methanol | | 7.37 (d, J = 2.6 Hz, 1H), 7.32 (dd, J = |
| | | | 9.2, 2.6 Hz, 1H), 7.21 (d, J = 5.8 Hz, |
| | | | 1H), 5.07 (t, J = 5.4 Hz, 1H), 4.49 (d, |
| | | | J = 6.0 Hz, 2H), 4.44 (d, J = 5.9 Hz, |
| | | | 2H), 4.32 (s, 2H), 3.77 (d, J = 5.4 Hz, |
| | | | 2H). |
| 330 | 1-(((1-((6-chloropyridin- | 379.7 | ¹H NMR (400 MHZ, DMSO-d₆) δ 9.58 |
| | 3-yl)amino)isoquinolin- | (M + 1), | (s, 1H), 8.87 (d, J = 2.7 Hz, 1H), 8.50 |
| | 6-yl)oxy)methyl)-2,2- | 381.6 | (d, J = 9.3 Hz, 1H), 8.41-8.39 (m, 1H), |
| | dimethylcyclopropane- | (M + 1). | 7.94 (d, J = 5.9 Hz, 1H), 7.49 (d, J = |
| | 1-carbonitrile 2,2,2- | | 8.7 Hz, 1H), 7.40 (dd, J = 9.2, 2.3 Hz, |
| | trifluoroacetate | | 1H), 7.34 (d, J = 2.3 Hz, 1H), 7.19 (d, |
| | | | J = 5.9 Hz, 1H), 4.50 (d, J = 10.9 Hz, |
| | | | 1H), 4.27 (d, J = 10.9 Hz, 1H), 1.37 |
| | | | (s, 3H), 1.33 (d, J = 5.2 Hz, 1H), 1.26 |
| | | | (d, J = 3.3 Hz, 4H), COOH not |
| | | | observed; ¹⁹F NMR (376 MHZ, |
| | | | DMSO-d₆) δ −73.7 (s). |
| 331 | 3-((1-((6-chloropyridin- | 422.0 | ¹H NMR (400 MHZ, DMSO-d₆) |
| | 3-yl)amino)isoquinolin- | (M + 1), | δ 8.78-8.78 (m, 1H), 8.51 (d, J = 9.2 |
| | 6-yl)oxy)adamantan-1-ol | 424.0 | Hz, 1H), 8.29-8.26 (m, 1H), 7.83-7.81 |
| | | (M + 1). | (m, 1H), 7.61-7.53 (m, 2H), 7.40-7.37 |
| | | | (m, 1H), 7.33-7.31 (m, 1H), 2.27-2.25 |
| | | | (m, 2H), 1.95-1.81 (m, 6H), 1.59-1.41 |
| | | | (m, 6H), OH and NH not observed. |
| 332 | N-(6-chloropyridin-3- | 366.2 | ¹H NMR (400 MHZ, DMSO-d₆) δ 9.37 |
| | yl)-6-(spiro[2.3]hexan- | (M + 1), | (d, J = 0.7 Hz, 1H), 8.89 (d, J = 2.7 |
| | 1-ylmethoxy)isoquinolin- | 368.2 | Hz, 1H), 8.45-8.41 (m, 2H), 7.96 (d, |
| | 1-amine | (M + 1). | J = 5.8 Hz, 1H), 7.45 (d, J = 8.7 Hz, |
| | | | 1H), 7.33-7.28 (m, 2H), 7.18 (d, J = |
| | | | 5.9 Hz, 1H), 4.08-4.04 (m, 1H), 3.89- |
| | | | 3.85 (m, 1H), 2.24-2.02 (m, 6H), 1.26- |
| | | | 1.19 (m, 1H), 0.77-0.74 (m, 1H), 0.47 |
| | | | (t, J = 5.2 Hz, 1H). |
| 333 | 6-(((1-((6-chloropyridin- | 407.1 | ¹H NMR (400 MHZ, CDCl₃) δ 8.52 (d, |
| | 3-yl)amino)isoquinolin- | (M + 1), | J = 2.7 Hz, 1H), 8.34 (dd, J = 8.7, 2.9 |
| | 6-yl)oxy)methyl)-2- | 409.1 | Hz, 1H), 8.04 (d, J = 5.9 Hz, 1H), 7.90 |
| | oxaspiro[3.3]heptane- | (M + 1). | (d, J = 9.1 Hz, 1H), 7.34 (d, J = 8.8 |
| | 6-carbonitrile | | Hz, 1H), 7.22 (dd, J = 9.2, 2.5 Hz, |
| | | | 1H), 7.12 (d, J = 5.9 Hz, 1H), 7.02 (d, |
| | | | J = 2.5 Hz, 1H), 4.13 (s, 2H), 3.97 (d, |
| | | | J = 21.5 Hz, 4H), 2.62 (dd, J = 6.7, |
| | | | 2.4 Hz, 2H), 2.23 (dd, J = 6.6, 2.6 Hz, |
| | | | 2H), NH not observed. |

### Example 334

### Synthesis of 6-((1H-pyrazol-4-yl)methoxy)-N-(6-chloropyridin-3-yl)isoquinolin-1-amine

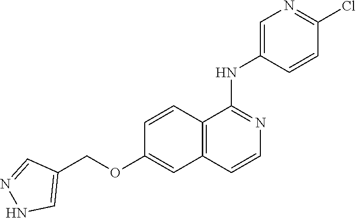 [SMILES: Clc1ccc(Nc2nccc3cc(OCc4cn[nH]c4)ccc23)cn1]

### Step 1. Preparation of ethyl 1-((2-(trimethylsilyl)ethoxy)methyl)-1H-pyrazole-4-carboxylate

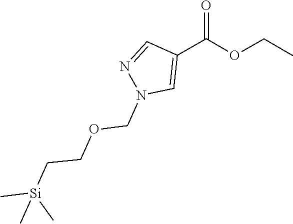

To a solution of ethyl pyrazole-4-carboxylate (2.00 g, 14.3 mmol) in tetrahydrofuran (28.5 mL) was added sodium hydride (60% dispersion in mineral oil, 0.685 g, 17.1 mmol) at 0° C. and the resulting mixture was stirred at 0° C. for 30 minutes. To it was then added 2-(trimethylsilyl)ethoxymethyl chloride (3.00 mL, 17.1 mmol). The reaction mixture was allowed to warm up to ambient temperature and stirred for 16 h. The reaction mixture was quenched by addition of 1 M sodium hydroxide solution (20 mL) and extracted with ethyl acetate (3×20 mL). The combined organic layers were dried over anhydrous sodium sulfate and filtered, and the filtrate was concentrated in vacuo. The obtained residue was purified by silica gel column chromatography, eluting with a gradient of 0 to 40% of ethyl acetate in heptane, to provide the title compound as a colorless oil (3.37 g, 87% yield): ¹H NMR (400 MHz, CDCl₃) δ8.05 (s, 1H), 7.93 (s, 1H), 5.42 (s, 2H), 4.30 (q, J=7.1 Hz, 2H), 3.57 (t, J=8.3 Hz, 2H), 1.34 (t, J=7.1 Hz, 3H), 0.93-0.87 (m, 2H), −0.03 (s, 9H); MS (ES+) m/z 271.6 (M+1).

### Step 2. Preparation of (1-((2-(trimethylsilyl)ethoxy)methyl)-1H-pyrazol-4-yl)methanol

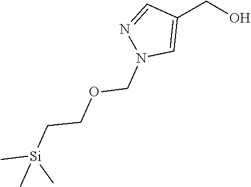

Following the procedure as described for EXAMPLE 151, Step 1, and making variations as required to replace 1-ethynylcyclopropane-1-carboxylic acid with ethyl 1-((2-(trimethylsilyl)ethoxy)methyl)-1H-pyrazole-4-carboxylate, the title compound was obtained as a colorless oil (2.63 g, 92% yield): ¹H NMR (400 MHz, CDCl₃) δ 7.55 (s, 1H), 7.53 (s, 1H), 5.38 (s, 2H), 4.60 (s, 2H), 3.57-3.52 (m, 2H), 0.91-0.87 (m, 2H), −0.03 (s, 9H), OH not observed; MS (ES+) m/z 229.2 (M+1).

### Step 3. Preparation of 6-((1H-pyrazol-4-yl)methoxy)-N-(6-chloropyridin-3-yl)isoquinolin-1-amine

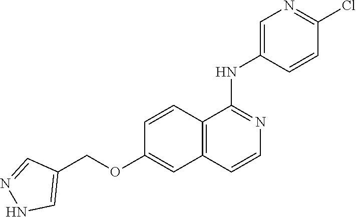

Following the procedure as described for EXAMPLE 279, Step 2, and making variations as required to replace (1-(difluoromethyl)-1H-pyrazol-4-yl)methanol with (1-((2-(trimethylsilyl)ethoxy)methyl)-1H-pyrazol-4-yl)methanol, the title compound was obtained as a colorless solid (0.0774 g, 22% yield): ¹H NMR (400 MHz, DMSO-d₆) δ 12.89 (br s, 1H), 9.51 (s, 1H), 8.86 (t, J=0.7 Hz, 1H), 8.45-8.38 (m, 2H), 7.93 (d, J=5.6 Hz, 1H), 7.79 (t, J=0.6 Hz, 2H), 7.47-7.42 (m, 2H), 7.29 (d, J=8.4 Hz, 1H), 7.21 (d, J=5.7 Hz, 1H), 5.15 (s, 2H); MS (ES+) m/z 352.0 (M+1), 354.0 (M+1).

### Example 335

### Synthesis of 6-(1-(1H-pyrazol-4-yl)ethoxy)-N-(6-chloropyridin-3-yl)isoquinolin-1-amine

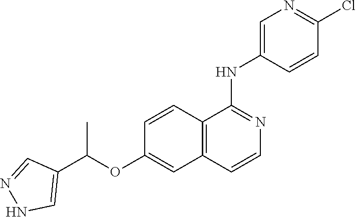

### Step 1. Preparation of 1-(1-((2-(trimethylsilyl)ethoxy)methyl)-1H-pyrazol-4-yl)ethan-1-one

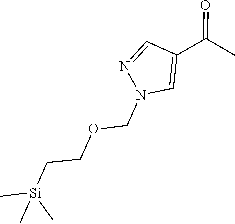

Following the procedure as described for EXAMPLE 334, Step 1, and making variations as required to replace ethyl pyrazole-4-carboxylate with 4-acetylpyrazole, the title compound was obtained as a colorless oil (0.934 g, 78% yield): ¹H NMR (400 MHz, CDCl₃) δ8.04 (s, 1H), 7.93 (s, 1H), 5.44 (s, 2H), 3.60-3.56 (m, 2H), 2.44 (s, 3H), 0.93-0.89 (m, 2H), −0.03 (s, 9H); MS (ES+) m/z 241.4 (M+1).

### Step 2. Preparation of 1-(1-((2-(trimethylsilyl)ethoxy)methyl)-1H-pyrazol-4-yl)ethan-1-ol

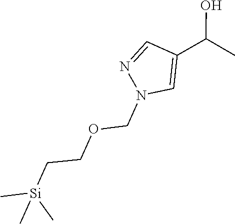

To a solution of 1-(1-((2-(trimethylsilyl)ethoxy)methyl)-1H-pyrazol-4-yl)ethan-1-one (0.930 g, 3.87 mmol) in methanol (39 mL) was added sodium borohydride (0.293 g, 7.74 mmol) at 0° C., and the resulting mixture was stirred at 0° C. for 1h. The mixture was quenched by addition of water (30 mL) and concentrated in vacuo. To the obtained residue was added ethyl acetate (100 mL) and the mixture was washed with saturated sodium bicarbonate solution (50 mL). The aqueous phase was extracted with ethyl acetate (3×100 mL), and the combined organic phases were washed with brine (100 mL), dried over anhydrous sodium sulfate, and filtered. The filtrate was concentrated in vacuo to provide the title compound as a colorless oil (0.946 g, quantitative yield): ¹H NMR (400 MHz, CDCl₃) δ7.55 (s, 2H), 5.41 (s, 2H), 4.95 (q, J=6.5 Hz, 1H), 3.58 (t, J=8.3 Hz, 2H), 1.54 (d, J=6.5 Hz, 3H), 0.93 (t, J=8.3 Hz, 2H), −0.00 (s, 9H), OH not observed; MS (ES+) m/z 243.4 (M+1).

### Step 3. Preparation of 6-(1-(1H-pyrazol-4-yl)ethoxy)-N-(6-chloropyridin-3-yl)isoquinolin-1-amine

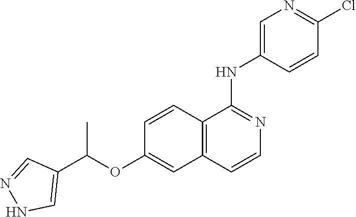

Following the procedure as described for EXAMPLE 279, Step 2, and making variations as required to replace (1-(difluoromethyl)-1H-pyrazol-4-yl)methanol with 1-(1-((2-(trimethylsilyl)ethoxy)methyl)-1H-pyrazol-4-yl)ethan-1-ol, the title compound was obtained as a colorless solid (0.004 g, 4% yield): ¹H NMR (400 MHz, CD₃CN) δ8.75 (d, J=2.8 Hz, 1H), 8.30 (dd, J=8.7, 2.9 Hz, 1H), 8.12-8.10 (m, 1H), 7.92 (d, J=5.8 Hz, 1H), 7.62 (s, 2H), 7.32 (d, J=8.7 Hz, 1H), 7.21 (dd, J=4.9, 2.4 Hz, 2H), 7.09 (d, J=5.7 Hz, 1H), 5.70 (q, J=6.4 Hz, 1H), 1.68 (d, J=6.4 Hz, 3H), two NH not observed; MS (ES+) m/z 366.2 (M+1), 368.2 (M+1).

### Example 336

### Synthesis of N-(6-chloropyridin-3-yl)-6-((1-(2-methoxyethyl)-1H-pyrazol-4-yl)methoxy)isoquinolin-1-amine

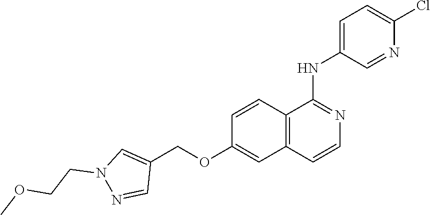

To the solution of 6-((1H-pyrazol-4-yl)methoxy)-N-(6-chloropyridin-3-yl)isoquinolin-1-amine (0.100 g, 0.284 mmol) in N,N-dimethylformamide (5.7 mL) was added 2-bromoethyl methyl ether (0.0290 mL, 0.313 mmol) and potassium carbonate (0.0480 g, 0.347 mmol). The reaction mixture was heated to 80° C. for 3 h. After cooling to ambient temperature, the mixture was concentrated in vacuo. The obtained residue was diluted with saturated sodium bicarbonate solution (20 mL) and extracted with ethyl acetate (3×20 mL). The combined organic layers were dried over anhydrous sodium sulfate and filtered, and the filtrate was concentrated in vacuo. The obtained residue was purified by silica gel column chromatography, eluting with a gradient of 0 to 100% of ethyl acetate in heptane, to provide the title compound as a colorless solid (0.004 g, 3% yield): ¹H NMR (400 MHz, DMSO-d₆) δ 8.30 (d, J=5.7 Hz, 1H), 7.86 (d, J=3.0 Hz, 1H), 7.58-7.54 (m, 4H), 7.38 (d, J=2.6 Hz, 1H), 7.24 (d, J=8.7 Hz, 1H), 7.15-7.10 (m, 2H), 5.10 (s, 2H), 4.24-4.22 (m, 2H), 3.72-3.69 (m, 2H), 3.31 (s, 3H), NH not observed; MS (ES+) m/z 410.2 (M+1), 412.2 (M+1).

### Example 337

### Synthesis of 6-((3-chloro-1H-pyrazol-4-yl)methoxy)-N-(6-chloropyridin-3-yl)isoquinolin-1-amine

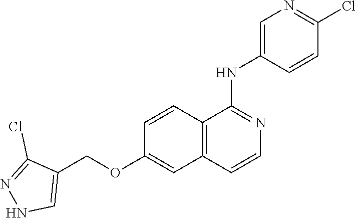 [SMILES: Clc1ccc(Nc2nccc3cc(OCc4c[nH]nc4Cl)ccc23)cn1]

### Step 1. Preparation of ethyl 3-chloro-1-((2-(trimethylsilyl)ethoxy)methyl)-1H-pyrazole-4-carboxylate

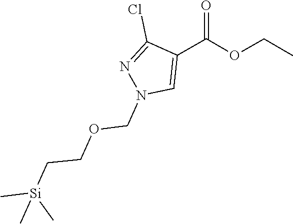

Following the procedure as described for EXAMPLE 334, Step 1, and making variations as required to replace ethyl pyrazole-4-carboxylate with ethyl 5-chloro-1H-pyrazole-4-carboxylate, the title compound was obtained as a colorless oil (0.131 g, 25% yield): ¹H NMR (400 MHz, CDCl₃) δ 8.04 (s, 1H), 5.35 (s, 2H), 4.32 (q, J=7.1 Hz, 2H), 3.61 (d, J=8.3 Hz, 2H), 1.36 (t, J=7.1 Hz, 3H), 0.92 (t, J=8.3 Hz, 2H), −0.01 (s, 9H); MS (ES+) m/z 305.2 (M+1), 307.2 (M+1).

### Step 2. Preparation of (3-chloro-1-((2-(trimethylsilyl)ethoxy)methyl)-1H-pyrazol-4-yl)methanol

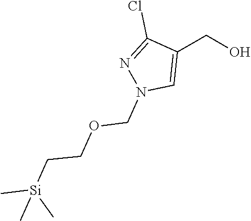

Following the procedure as described for EXAMPLE 151, Step 1, and making variations as required to replace 1-ethynylcyclopropane-1-carboxylic acid with ethyl 3-chloro-1-((2-(trimethylsilyl)ethoxy)methyl)-1H-pyrazole-4-carboxylate, the title compound was obtained as a colorless oil (0.102 g, 90% yield): ¹H NMR (400 MHz, CDCl₃) δ7.59 (s, 1H), 5.35 (s, 2H), 4.60 (s, 2H), 3.62-3.58 (m, 2H), 0.95-0.91 (m, 2H), 0.01 (s, 9H), OH not observed; MS (ES+) m/z 263.6 (M+1), 265.6 (M+1).

### Step 3. Preparation of 6-((3-chloro-1H-pyrazol-4-yl)methoxy)-N-(6-chloropyridin-3-yl)isoquinolin-1-amine

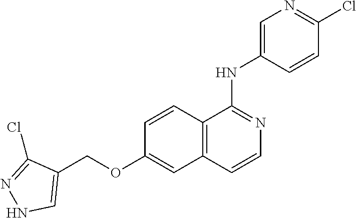 [SMILES: Clc1ccc(Nc2nccc3cc(OCc4c[nH]nc4Cl)ccc23)cn1]

To a solution of (3-chloro-1-((2-(trimethylsilyl)ethoxy)methyl)-1H-pyrazol-4-yl)methanol (0.036 g, 0.136 mmol) in N,N-dimethylformamide (1 mL) was added N-(6-chloropyridin-3-yl)-6-fluoro-N-((2-(trimethylsilyl)ethoxy)methyl)isoquinolin-1-amine (0.050 g, 0.124 mmol) and a 1 M solution of potassium tert-butoxide in tetrahydrofuran (0.186 mL, 0.186 mmol) at ambient temperature, and the resulting mixture was heated to 80° C. for 1 h. After cooling to ambient temperature, the mixture was concentrated in vacuo. To the obtained residue was added dichloromethane (1 mL) followed by trifluoroacetic acid (0.380 mL, 4.95 mmol) and the mixture was heated to reflux for 6 h. The mixture was allowed to cool to ambient temperature, and saturated sodium bicarbonate solution (20 mL) was added to it. The mixture was extracted with dichloromethane (3×20 mL). The combined organic phase was washed with brine (20 mL), dried over anhydrous sodium sulfate, and filtered. The filtrate was concentrated in vacuo to provide a residue which was purified by reverse phase preparative HPLC (HSS PFP, 30×75 mm column), eluting with a gradient of 41 to 61% of acetonitrile in water (containing 10 mM of ammonium formate), to afford the title compound as a colorless solid (0.0024 g, 5% yield): ¹H NMR (400 MHz, DMSO-d₆) δ13.22 (br s, 1H), 9.39 (s, 1H), 8.88 (d, J=2.5 Hz, 1H), 8.45-8.41 (m, 2H), 8.09 (s, 1H), 7.98-7.97 (m, 1H), 7.46-7.44 (m, 2H), 7.30-7.28 (m, 1H), 7.20 (d, J=5.9 Hz, 1H), 5.07 (d, J=0.1 Hz, 2H); MS (ES+) m/z 386.0 (M+1), 388.0 (M+1).

### Example 338

### Synthesis of N-(6-chloropyridin-3-yl)-6-((3,5-dimethyl-1H-pyrazol-4-yl)methoxy)isoquinolin-1-amine

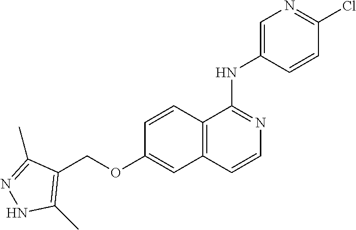 [SMILES: Cc1n[nH]c(C)c1COc1ccc2c(Nc3ccc(Cl)nc3)nccc2c1]

### Step 1. Preparation of ethyl 3,5-dimethyl-1-((2-(trimethylsilyl)ethoxy)methyl)-1H-pyrazole-4-carboxylate

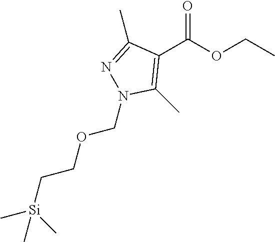 [SMILES: CCOC(=O)c1c(C)nn(COCC[Si](C)(C)C)c1C]

Following the procedure as described for EXAMPLE 334, Step 1 and making variations as required to replace ethyl pyrazole-4-carboxylate with ethyl 3,5-dimethyl-1H-4-pyrazolecarboxylate, the title compound was obtained as a colorless oil (0.400 g, 78% yield): ¹H NMR (400 MHz, CDCl₃) δ5.36 (s, 2H), 4.29 (q, J=7.1 Hz, 2H), 3.58-3.54 (m, 2H), 2.57 (s, 3H), 2.42 (s, 3H), 1.36 (t, J=7.1 Hz, 3H), 0.91-0.87 (m, 2H), −0.03 (s, 9H); MS (ES+) m/z 299.4 (M+1).

### Step 2. Preparation of (3,5-dimethyl-1-((2-(trimethylsilyl)ethoxy)methyl)-1H-pyrazol-4-yl)methanol

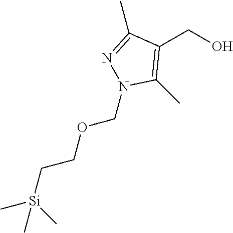 [SMILES: Cc1nn(COCC[Si](C)(C)C)c(C)c1CO]

Following the procedure as described for EXAMPLE 151, Step 1, and making variations as required to replace 1-ethynylcyclopropane-1-carboxylic acid with ethyl 3,5-dimethyl-1-((2-(trimethylsilyl)ethoxy)methyl)-1H-pyrazole-4-carboxylate, the title compound was obtained as a yellow oil (0.331 g, 96% yield): ¹H NMR (400 MHz, CDCl₃) δ5.36-5.35 (m, 2H), 4.51 (d, J=0.9 Hz, 2H), 3.61-3.56 (m, 2H), 2.34 (s, 3H), 2.29 (s, 3H), 0.91 (t, J=8.3 Hz, 2H), −0.00 (s, 9H), OH not observed; MS (ES+) m/z 257.6 (M+1).

### Step 3. Preparation of N-(6-chloropyridin-3-yl)-6-((3,5-dimethyl-1H-pyrazol-4-yl)methoxy)isoquinolin-1-amine

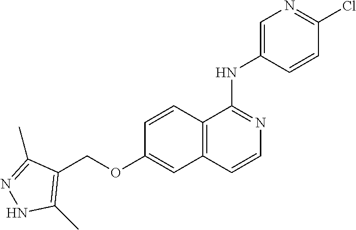 [SMILES: Cc1n[nH]c(C)c1COc1ccc2c(Nc3ccc(Cl)nc3)nccc2c1]

Following the procedure as described for EXAMPLE 337, Step 3 and making variations as required to replace (3-chloro-1-((2-(trimethylsilyl)ethoxy)methyl)-1H-pyrazol-4-yl)methanol with (3,5-dimethyl-1-((2-(trimethylsilyl)ethoxy)methyl)-1H-pyrazol-4-yl)methanol, the title compound was obtained as a colorless solid (0.020 g, 27% yield): ¹H NMR (400 MHz, DMSO-d₆) δ 10.08 (br s, 1H), 8.79 (d, J=2.7 Hz, 1H), 8.50 (d, J=9.3 Hz, 1H), 8.30-8.27 (m, 1H), 7.83-7.81 (m, 1H), 7.59-7.57 (m, 1H), 7.51-7.51 (m, 1H), 7.39-7.36 (m, 1H), 7.25 (d, J=6.2 Hz, 1H), 5.06 (s, 2H), 2.20 (s, 6H), one NH not observed; MS (ES+) m/z 380.2 (M+1), 382.2 (M+1).

### Examples 339 and 340

### Synthesis of (1R,3S)-3-((1-((2-methylpyrimidin-5-yl)amino)isoquinolin-6-yl)oxy)cyclohexan-1-ol and (1 S,3R)-3-((1-((2-methylpyrimidin-5-yl)amino)isoquinolin-6-yl)oxy)cyclohexan-1-ol

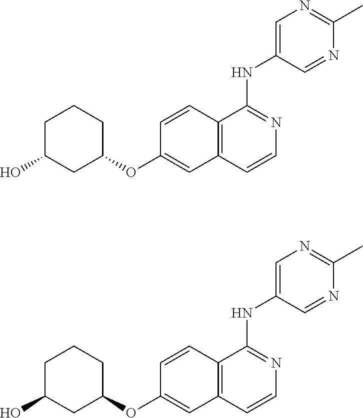 [SMILES: Cc1ncc(Nc2nccc3cc(O[C@H]4CCC[C@@H](O)C4)ccc23)cn1 ; Cc1ncc(Nc2nccc3cc(O[C@@H]4CCC[C@H](O)C4)ccc23)cn1]

Following the procedure as described for EXAMPLE 228, Step 2, making variations as required to replace 3-(hydroxymethyl)oxetane-3-carbonitrile with cis-1,3-cyclohexanediol, provided cis-3-((1-((2-methylpyrimidin-5-yl)amino)isoquinolin-6-yl)oxy)cyclohexan-1-ol as a mixture of enantiomers. Resolution of the enantiomers by chiral SFC (ChiralPak IA, 10×250 mm, 5 μm column), eluting with 50% of methanol (containing 10 mM of ammonium formate) in supercritical carbon dioxide, afforded the title compounds as single enantiomers as colorless solids. First eluting enantiomer (0.043 g, 15% yield): ¹H NMR (400 MHz, DMSO-d₆) δ9.29 (s, 1H), 9.16 (s, 2H), 8.41 (d, J=9.3 Hz, 1H), 7.93 (d, J=5.8 Hz, 1H), 7.33 (d, J=2.5 Hz, 1H), 7.25 (dd, J=9.1, 2.5 Hz, 1H), 7.16 (d, J=5.9 Hz, 1H), 4.77-4.76 (m, 1H), 4.56-4.49 (m, 1H), 3.63-3.56 (m, 1H), 2.58 (s, 3H), 2.39-2.32 (m, 1H), 2.12-2.07 (m, 1H), 1.88-1.83 (m, 1H), 1.78-1.73 (m, 1H), 1.41-1.20 (m, 3H), 1.20-1.07 (m, 1H); MS (ES+) m/z 351.0 (M+1). Second eluting enantiomer (0.045 g, 15% yield): ¹H NMR (400 MHz, DMSO-d₆) δ9.29 (s, 1H), 9.16 (s, 2H), 8.41 (d, J=9.2 Hz, 1H), 7.93 (d, J=5.8 Hz, 1H), 7.32 (d, J=2.5 Hz, 1H), 7.25 (dd, J=9.2, 2.5 Hz, 1H), 7.16 (d, J=5.8 Hz, 1H), 4.78-4.75 (m, 1H), 4.56-4.49 (m, 1H), 3.63-3.56 (m, 1H), 2.58 (s, 3H), 2.37-2.33 (m, 1H), 2.12-2.07 (m, 1H), 1.88-1.83 (m, 1H), 1.78-1.73 (m, 1H), 1.41-1.21 (m, 3H), 1.20-1.07 (m, 1H); MS (ES+) m/z 351.0 (M+1).

### Example 341

### Synthesis of N-(6-chloropyridin-3-yl)-6-(2-(3-methyloxetan-3-yl)ethyl)isoquinolin-1-amine

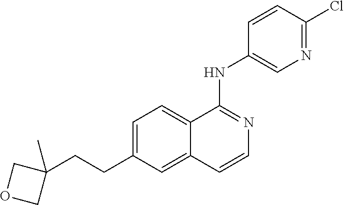 [SMILES: CC1(CCc2ccc3c(Nc4ccc(Cl)nc4)nccc3c2)COC1]

### Step 1. Preparation of 6-((3-methyloxetan-3-yl)ethynyl)isoquinolin-1-ol

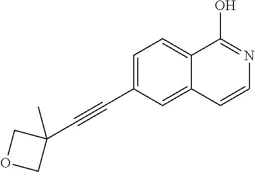 [SMILES: CC1(C#Cc2ccc3c(O)nccc3c2)COC1]

To a solution of 6-bromoisoquinolin-1-ol (0.500 g, 2.23 mmol), triethylamine (0.677 g, 6.69 mmol), and 3-ethynyl-3-methyloxetane (0.321 g, 3.35 mmol) in N,N-dimethylformamide (5 mL) was added copper(I) iodide (0.085 g, 0.446 mmol) and bis(triphenylphosphine)palladium(II) dichloride (0.156 g, 0.223 mmol). The reaction mixture was stirred at ambient temperature for 12h. The mixture was then poured into water (20 mL) and extracted with dichloromethane (3×5 mL). The combined organic phase was washed with brine (5 mL), dried over anhydrous sodium sulfate, and filtered. Concentration of the filtrate in vacuo provided a residue which was purified by preparative thin layer chromatography, eluting with 5% of methanol in dichloromethane, to provide the title compound as a yellow solid (0.196 g, 37% yield): MS (ES+) m/z 240.1 (M+1).

### Step 2. Preparation of 6-(2-(3-methyloxetan-3-yl)ethyl)isoquinolin-1-yl trifluoromethanesulfonate

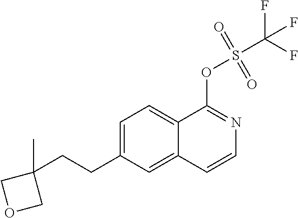 [SMILES: CC1(CCc2ccc3c(OS(=O)(=O)C(F)(F)F)nccc3c2)COC1]

To a solution of 6-((3-methyloxetan-3-yl)ethynyl)isoquinolin-1-ol (0.196 g, 0.819 mmol) in methanol (2 mL) was added 10% palladium on activated carbon (0.0050 g, 0.00470 mmol). The mixture was degassed and purged with hydrogen three times. The reaction mixture was stirred under hydrogen atmosphere (15 psi) at ambient temperature for 12 h. Filtration and concentration of the filtrate in vacuo provided a colorless solid (0.160 g). To a solution of the obtained residue in pyridine (2 mL) was added dropwise trifluoromethanesulfonic anhydride (0.278 g, 0.986 mmol) at ambient temperature. The reaction mixture was stirred at ambient temperature for 2 h and then concentrated in vacuo. The obtained residue was purified by preparative thin layer chromatography, eluting with 5% of methanol in dichloromethane, to provide the title compound as a yellow solid (0.120 g, 39% yield).

### Step 3. Preparation of N-(6-chloropyridin-3-yl)-6-(2-(3-methyloxetan-3-yl)ethyl)isoquinolin-1-amine

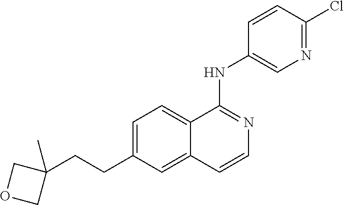 [SMILES: CC1(CCc2ccc3c(Nc4ccc(Cl)nc4)nccc3c2)COC1]

To a mixture of 6-(2-(3-methyloxetan-3-yl)ethyl)isoquinolin-1-yl trifluoromethanesulfonate (0.100 g, 0.266 mmol) and 6-chloropyridin-3-amine (0.0411 g, 0.319 mmol) in toluene (5 mL) was added tris(dibenzylideneacetone)dipalladium(0) (0.024 g, 0.0266 mmol), 2-dicyclohexylphosphino-2′,6′-dimethoxy-1,1′-biphenyl (0.022 g, 0.0532 mmol), and potassium phosphate tribasic (0.113 g, 0.532 mmol). The mixture was degassed by passing a stream of nitrogen through it for 5 minutes and then heated to 100° C. for 12 h. After cooling to ambient temperature, the reaction mixture was filtered through a pad of celite, and the filtrate was concentrated in vacuo. The residue was purified by silica gel column chromatography, eluting with a gradient of 3 to 50% of ethyl acetate in petroleum ether, to provide the title compound as a yellow solid (0.032 g, 16% yield): ¹H NMR (400 MHz, DMSO-d₆) δ8.54 (d, J=2.4 Hz, 1H), 8.42 (dd, J=8.8, 2.8 Hz, 1H), 8.08 (d, J=6.0 Hz, 1H), 7.92-7.85 (m, 1H), 7.60 (s, 1H), 7.47 (d, J=8.8 Hz, 1H), 7.33 (d, J=8.4 Hz, 1H), 7.19-7.08 (m, 2H), 4.55-4.34 (m, 4H), 2.82-2.75 (m, 2H), 2.12-2.05 (m, 2H), 1.43 (s, 3H); MS (ES+) m/z 354.0 (M+1), 356 (M+1).

### Examples 342-345

### Synthesis of (1 S,3S)-3-((1-((6-chloropyridin-3-yl)amino)isoquinolin-6-yl)oxy)cyclohexane-1-carbonitrile, (1R,3S)-3-((1-((6-chloropyridin-3-yl)amino)isoquinolin-6-yl)oxy)cyclohexane-1-carbonitrile, (1R,3R)-3-((1-((6-chloropyridin-3-yl)amino)isoquinolin-6-yl)oxy)cyclohexane-1-carbonitrile, and (1 S,3R)-3-((1-((6-chloropyridin-3-yl)amino)isoquinolin-6-yl)oxy)cyclohexane-1-carbonitrile

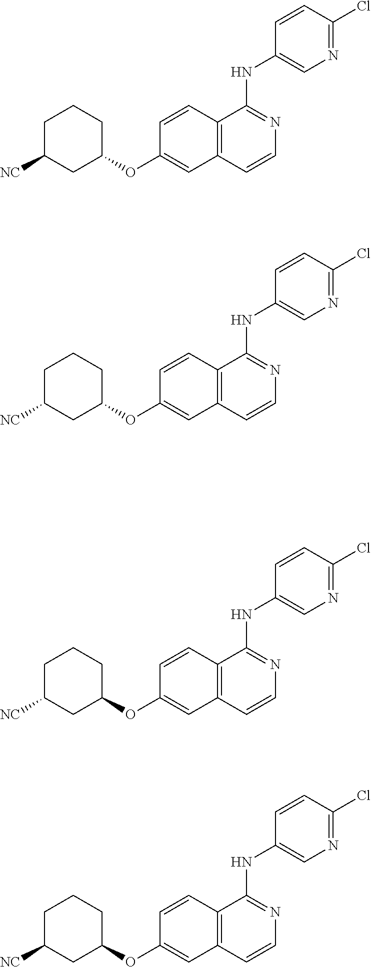 [SMILES: N#C[C@H]1CCC[C@H](Oc2ccc3c(Nc4ccc(Cl)nc4)nccc3c2)C1 ; N#C[C@@H]1CCC[C@H](Oc2ccc3c(Nc4ccc(Cl)nc4)nccc3c2)C1 ; N#C[C@@H]1CCC[C@@H](Oc2ccc3c(Nc4ccc(Cl)nc4)nccc3c2)C1 ; N#C[C@H]1CCC[C@@H](Oc2ccc3c(Nc4ccc(Cl)nc4)nccc3c2)C1]

Following the procedure as described for EXAMPLE 76, Step 3, and making variations as required to replace phenylmethanol with 3-hydroxycyclohexane-1-carbonitrile, a mixture of the title compounds was obtained. Separation of the mixture by chiral SFC (ChiralPak OJ, 10×250 mm, 5 μm column), eluting with 45% of propan-2-ol (containing 10 mM ammonium formate) in supercritical carbon dioxide, afforded the title compounds as single enantiomers as colorless solids. First eluting enantiomer (0.0065 g, 11% yield): ¹H NMR (400 MHz, CD₃CN) δ8.80 (d, J=2.7 Hz, 1H), 8.35 (dd, J=8.7, 2.8 Hz, 1H), 8.18 (d, J=8.7 Hz, 1H), 7.97 (d, J=5.8 Hz, 2H), 7.36 (d, J=8.7 Hz, 1H), 7.27-7.24 (m, 2H), 7.16 (d, J=5.8 Hz, 1H), 4.91-4.80 (m, 1H), 3.18-3.11 (m, 1H), 2.21-2.07 (m, 2H), 1.94-1.69 (m, 6H); MS (ES+) m/z 379.2 (M+1), 381.2 (M+1). Second eluting enantiomer (0.0052 g, 9% yield): ¹H NMR (400 MHz, CD₃CN) δ 8.80 (d, J=2.7 Hz, 1H), 8.35 (dd, J=8.7, 2.9 Hz, 1H), 8.17 (d, J=9.0 Hz, 1H), 7.98 (t, J=5.0 Hz, 2H), 7.37 (d, J=8.6 Hz, 1H), 7.26-7.22 (m, 2H), 7.16 (d, J=5.9 Hz, 1H), 4.57-4.50 (m, 1H), 2.90-2.82 (m, 1H), 2.49-2.44 (m, 1H), 2.15-2.12 (m, 1H), 2.06-2.01 (m, 1H), 1.94-1.91 (m, 1H), 1.85-1.77 (m, 1H), 1.69-1.51 (m, 3H); MS (ES+) m/z 379.2 (M+1), 381.2 (M+1). Third eluting enantiomer (0.0062 g, 11% yield): ¹H NMR (400 MHz, CD₃CN) δ 8.80 (d, J=2.7 Hz, 1H), 8.35 (dd, J=8.7, 2.9 Hz, 1H), 8.19-8.17 (m, 1H), 7.99-7.97 (m, 2H), 7.37 (d, J=8.7 Hz, 1H), 7.28-7.25 (m, 2H), 7.16 (d, J=5.7 Hz, 1H), 4.88-4.83 (m, 1H), 3.18-3.11 (m, 1H), 2.15-2.07 (m, 2H), 1.93-1.69 (m, 6H); MS (ES+) m/z 379.2 (M+1), 381.2 (M+1). Forth eluting enantiomer (0.006 g, 10% yield): ¹H NMR (400 MHz, CD₃CN) δ8.81-8.80 (m, 1H), 8.36-8.33 (m, 1H), 8.17 (d, J=9.0 Hz, 1H), 7.99-7.97 (m, 2H), 7.37 (d, J=8.7 Hz, 1H), 7.26-7.22 (m, 2H), 7.16 (d, J=5.8 Hz, 1H), 4.57-4.50 (m, 1H), 2.90-2.82 (m, 1H), 2.50-2.44 (m, 1H), 2.15-2.11 (m, 1H), 2.07-2.01 (m, 1H), 1.95-1.91 (m, 1H), 1.85-1.77 (m, 1H), 1.66-1.51 (m, 3H); MS (ES+) m/z 379.2 (M+1), 381.2 (M+1).

### Examples 346 and 347

### Synthesis of (1R,3S)-3-((1-((6-chloropyridin-3-yl)amino)isoquinolin-6-yl)oxy)cyclohexan-1-ol and (1 S,3R)-3-((1-((6-chloropyridin-3-yl)amino)isoquinolin-6-yl)oxy)cyclohexan-1-ol

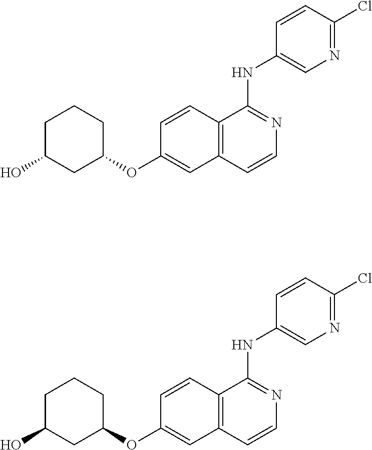 [SMILES: O[C@@H]1CCC[C@H](Oc2ccc3c(Nc4ccc(Cl)nc4)nccc3c2)C1 ; O[C@H]1CCC[C@@H](Oc2ccc3c(Nc4ccc(Cl)nc4)nccc3c2)C1]

Following the procedure as described for EXAMPLE 76, Step 3, and making variations as required to replace phenylmethanol with cis-1,3-cyclohexanediol, a mixture of the title compounds was obtained. Resolution of the enantiomers by chiral SFC (ChiralPak IA, 10×250 mm, 5 μm column), eluting with 50% of methanol (containing 10 mM ammonium formate) in supercritical carbon dioxide, afforded the title compounds as single enantiomers as colorless solids. First eluting enantiomer (0.048 g, 11% yield): ¹H NMR (400 MHz, DMSO-d₆) δ 9.37 (s, 1H), 8.88 (d, J=2.8 Hz, 1H), 8.43 (dd, J=8.8, 2.7 Hz, 2H), 7.95 (d, J=5.8 Hz, 1H), 7.45 (d, J=8.7 Hz, 1H), 7.33 (d, J=2.5 Hz, 1H), 7.25 (dd, J=9.2, 2.5 Hz, 1H), 7.18 (d, J=5.8 Hz, 1H), 4.76 (t, J=3.6 Hz, 1H), 4.56-4.48 (m, 1H), 3.64-3.55 (m, 1H), 2.38-2.33 (m, 1H), 2.12-2.07 (m, 1H), 1.88-1.83 (m, 1H), 1.78-1.73 (m, 1H), 1.41-1.07 (m, 4H); MS (ES+) m/z 370.2 (M+1), 372.2 (M+1). Second eluting enantiomer (0.045 g, 10% yield): ¹H NMR (400 MHz, DMSO-d₆) δ 9.37 (s, 1H), 8.88 (d, J=2.8 Hz, 1H), 8.43 (dd, J=8.8, 2.8 Hz, 2H), 7.95 (d, J=5.8 Hz, 1H), 7.45 (d, J=8.7 Hz, 1H), 7.33 (d, J=2.5 Hz, 1H), 7.25 (dd, J=9.2, 2.6 Hz, 1H), 7.18 (d, J=5.9 Hz, 1H), 4.77-4.76 (m, 1H), 4.56-4.48 (m, 1H), 3.63-3.56 (m, 1H), 2.38-2.33 (m, 1H), 2.12-2.07 (m, 1H), 1.88-1.83 (m, 1H), 1.78-1.73 (m, 1H), 1.41-1.07 (m, 4H); MS (ES+) m/z 370.2 (M+1), 372.2 (M+1).

### Example 348

### Synthesis of cis-3-((1-((2-chloropyrimidin-5-yl)amino)isoquinolin-6-yl)oxy)cyclohexan-1-ol

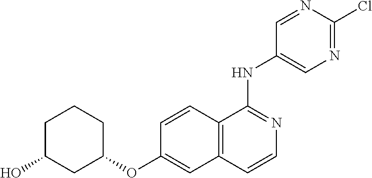 [SMILES: O[C@@H]1CCC[C@H](Oc2ccc3c(Nc4cnc(Cl)nc4)nccc3c2)C1]

### Step 1. Preparation of cis-3-((1-chloroisoquinolin-6-yl)oxy)cyclohexan-1-ol

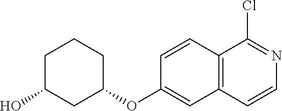 [SMILES: O[C@@H]1CCC[C@H](Oc2ccc3c(Cl)nccc3c2)C1]

To a solution of cis-1,3-cyclohexanediol (0.450 g, 3.87 mmol) in N,N-dimethylformamide (15 mL) was added sodium hydride (60% dispersion in mineral oil, 0.82 g, 4.54 mmol). The reaction mixture was stirred at ambient temperature for 5 minutes, and to it was then added 1-chloro-6-fluoroisoquinoline (0.550 g, 3.03 mmol). The reaction was stirred at 0° C. for 3 h. The mixture was diluted with ethyl acetate (50 mL) and washed with saturated aqueous sodium bicarbonate (50 mL) and brine (50 mL). The organic phase was dried over anhydrous sodium sulfate and filtered. The filtrate was concentrated in vacuo to give a residue, which was purified by silica gel column chromatography, eluting with a gradient of 0 to 80% of ethyl acetate in heptane, to afford the title compound as a colorless solid (0.800 g, 82% yield): ¹H NMR (400 MHz, DMSO-d₆) δ8.19 (d, J=5.7 Hz, 1H), 8.15 (d, J=9.2 Hz, 1H), 7.77-7.75 (m, 1H), 7.55 (d, J=2.5 Hz, 1H), 7.40 (dd, J=9.2, 2.5 Hz, 1H), 4.78-4.77 (m, 1H), 4.59-4.51 (m, 1H), 3.62-3.56 (m, 1H), 2.38-2.33 (m, 1H), 2.13-2.08 (m, 1H), 1.88-1.83 (m, 1H), 1.78-1.73 (m, 1H), 1.38-1.23 (m, 3H), 1.16-1.07 (m, 1H); MS (ES+) m/z 278.6 (M+1), 280.6 (M+1).

### Step 2. Preparation of cis-3-((1-((2-chloropyrimidin-5-yl)amino)isoquinolin-6-yl)oxy)cyclohexan-1-ol

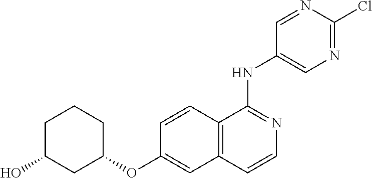 [SMILES: O[C@@H]1CCC[C@H](Oc2ccc3c(Nc4cnc(Cl)nc4)nccc3c2)C1]

To a solution of cis-3-((1-chloroisoquinolin-6-yl)oxy)cyclohexan-1-ol (0.215 g, 0.774 mmol) in 1,4-dioxane (6 mL) was added 5-amino-2-chloropyrimidine (0.100 g, 0.774 mmol), 2-dicyclohexylphosphino-2′,6′-dimethoxy-1,1-biphenyl (0.048 g, 0.116 mmol), tris(dibenzylideneacetone)dipalladium(0) (0.053 g, 0.0581 mmol), and potassium phosphate tribasic (0.246 g, 1.16 mmol). The reaction mixture was degassed for 5 minutes and then heated to 110° C. for 1 h. After cooling to ambient temperature, the reaction mixture was filtered through celite, and the filtrate was concentrated in vacuo. The obtained residue was purified by silica gel column chromatography, eluting with a gradient of 0 to 20% of methanol in ethyl acetate, to provide the title compound as a colourless solid (0.280 g, 79% yield): ¹H NMR (400 MHz, DMSO-d₆) δ 9.58-9.58 (m, 1H), 9.30 (s, 2H), 8.43-8.41 (m, 1H), 7.99 (d, J=5.8 Hz, 1H), 7.36 (d, J=2.5 Hz, 1H), 7.29 (dd, J=9.2, 2.6 Hz, 1H), 7.25-7.23 (m, 1H), 4.80-4.74 (m, 1H), 4.57-4.49 (m, 1H), 3.63-3.56 (m, 1H), 2.39-2.33 (m, 1H), 2.12-2.07 (m, 1H), 1.89-1.83 (m, 1H), 1.78-1.73 (m, 1H), 1.41-1.07 (m, 4H); MS (ES+) m/z 371.2 (M+1), 373.2 (M+1).

### Examples 349-357

In a similar manner as described in EXAMPLE 348, utilizing the appropriately substituted starting materials and intermediates, the following compounds were prepared:

| | | MS | |
|---|---|---|---|
| Example | | (ES+) | |
| No. | Name | m/z | NMR |
| 349 | 1-(((1-((2- | 378.0 | ¹H NMR (400 MHZ, DMSO-d₆) δ 9.61 |
| | chloropyrimidin-5- | (M + 1), | (s, 1H), 9.31 (s, 2H), 8.46 (d, J = 9.3 |
| | yl)amino)isoquinolin-6- | 380.0, | Hz, 1H), 8.02 (d, J = 5.8 Hz, 1H), |
| | yl)oxy)methyl)spiro[2.2] | (M + 1). | 7.41-7.38 (m, 1H), 7.33 (d, J = 2.5 Hz, |
| | pentane-1-carbonitrile | | 1H), 7.24 (d, J = 5.8 Hz, 1H), 4.34- |
| | | | 4.26 (m, 2H), 1.91 (d, J = 5.0 Hz, 1H), |
| | | | 1.74 (d, J = 4.9 Hz, 1H), 1.20-1.15 (m, |
| | | | 1H), 1.11-1.06 (m, 2H), 1.04-1.00 (m, 1H). |
| 350 | (1r,4r)-4-((1-((6- | 370.2 | ¹H NMR (400 MHZ, DMSO-d₆) δ 9.36 |
| | chloropyridin-3- | (M + 1), | (s, 1H), 8.88 (d, J = 2.8 Hz, 1H), 8.44- |
| | yl)amino)isoquinolin-6- | 372.2 | 8.40 (m, 2H), 7.95 (d, J = 5.8 Hz, 1H), |
| | yl)oxy)cyclohexan-1-ol | (M + 1). | 7.45 (d, J = 8.8 Hz, 1H), 7.32 (d, J = |
| | | | 2.5 Hz, 1H), 7.24 (dd, J = 9.1, 2.5 Hz, |
| | | | 1H), 7.18 (d, J = 5.8 Hz, 1H), 4.64 |
| | | | (dd, J = 4.2, 2.0 Hz, 1H), 4.57-4.52 |
| | | | (m, 1H), 3.59-3.53 (m, 1H), 2.12-2.06 |
| | | | (m, 2H), 1.90-1.84 (m, 2H), 1.53-1.35 |
| | | | (m, 4H). |
| 351 | 1-(((1-((2- | 380.0 | ¹H NMR (400 MHZ, DMSO-d₆) δ 9.62 |
| | chloropyrimidin-5- | (M + 1), | (s, 1H), 9.31 (s, 2H), 8.47 (d, J = 9.3 |
| | yl)amino)isoquinolin-6- | 382.0 | Hz, 1H), 8.02 (d, J = 5.8 Hz, 1H), 7.42 |
| | yl)oxy)methyl)-2,2- | (M + 1) | (dd, J = 9.2, 2.5 Hz, 1H), 7.34 (d, J = |
| | dimethylcyclopropane- | | 2.6 Hz, 1H), 7.23 (d, J = 5.7 Hz, 1H), |
| | 1-carbonitrile | | 4.50 (d, J = 10.9 Hz, 1H), 4.27 (d, J = |
| | | | 10.9 Hz, 1H), 1.37 (s, 3H), 1.33 (d, J = |
| | | | 5.2 Hz, 1H), 1.26 (d, J = 3.6 Hz, 4H). |
| 352 | N-(2-chloropyrimidin-5- | 381.0 | ¹H NMR (400 MHZ, DMSO-d₆) δ 9.57 |
| | yl)-6-((1,5-dimethyl-1H- | (M + 1), | (s, 1H), 9.29 (s, 2H), 8.41 (d, J = 9.3 |
| | pyrazol-4- | 383.0 | Hz, 1H), 8.00 (d, J = 5.8 Hz, 1H), 7.49 |
| | yl)methoxy)isoquinolin- | (M + 1). | (s, 1H), 7.44 (d, J = 2.6 Hz, 1H), 7.30 |
| | 1-amine | | (dd, J = 9.2, 2.6 Hz, 1H), 7.25 (d, J = |
| | | | 5.8 Hz, 1H), 5.06 (s, 2H), 3.74 (s, 3H), |
| | | | 2.28 (s, 3H). |
| 353 | 6-((1,5-dimethyl-1H- | 361.0 | ¹H NMR (400 MHZ, DMSO-d₆) δ 9.30 |
| | pyrazol-4-yl)methoxy)- | (M + 1). | (s, 1H), 9.16 (s, 2H), 8.41 (d, J = 9.3 |
| | N-(2-methylpyrimidin-5- | | Hz, 1H), 7.95 (d, J = 5.8 Hz, 1H), 7.50 |
| | yl)isoquinolin-1-amine | | (s, 1H), 7.41 (d, J = 2.5 Hz, 1H), 7.26 |
| | | | (dd, J = 9.2, 2.5 Hz, 1H), 7.18 (d, J = |
| | | | 5.8 Hz, 1H), 5.06 (s, 2H), 3.74 (s, 3H), |
| | | | 2.58 (s, 3H), 2.29 (s, 3H). |
| 354 | trans-3-((1-((6- | 370.2 | ¹H NMR (400 MHZ, DMSO-d₆) δ 9.37 |
| | chloropyridin-3- | (M + 1), | (s, 1H), 8.88 (d, J = 2.8 Hz, 1H), 8.43 |
| | yl)amino)isoquinolin-6- | 372.2 | (dd, J = 8.8, 2.7 Hz, 2H), 7.95 (d, J = |
| | yl)oxy)cyclohexan-1-ol | (M + 1). | 5.8 Hz, 1H), 7.45 (d, J = 8.7 Hz, 1H), |
| | | | 7.28-7.23 (m, 2H), 7.18 (d, J = 5.8 Hz, |
| | | | 1H), 4.91-4.86 (m, 1H), 4.64 (s, 1H), |
| | | | 3.95-3.90 (m, 1H), 1.91-1.76 (m, 3H), |
| | | | 1.71-1.57 (m, 4H), 1.46-1.38 (m, 1H). |
| 355 | N-(2-chloropyrimidin-5- | 407.0 | ¹H NMR (400 MHZ, DMSO-d₆) δ 9.62 |
| | yl)-6-((3-(1,1- | (M + 1), | (s, 1H), 9.31 (s, 2H), 8.48 (d, J = 9.3 |
| | difluoroethyl)oxetan-3- | 409.0 | Hz, 1H), 8.03 (d, J = 5.8 Hz, 1H), 7.49 |
| | yl)methoxy)isoquinolin- | (M + 1). | (d, J = 2.5 Hz, 1H), 7.42 (dd, J = 9.2, |
| | 1-amine | | 2.6 Hz, 1H), 7.26 (d, J = 5.8 Hz, 1H), |
| | | | 4.66 (q, J = 6.3 Hz, 4H), 4.44 (s, 2H), |
| | | | 1.76 (t, J = 19.8 Hz, 3H). |
| 356 | tert-butyl ((1S,3R)-3- | 470.0 | ¹H NMR (400 MHZ, DMSO-d₆) δ 9.56 |
| | ((1-((2-chloropyrimidin- | (M + 1), | (s, 1H), 9.30 (s, 2H), 8.41 (d, J = 9.3 |
| | 5-yl)amino)isoquinolin- | 472.0 | Hz, 1H), 7.99 (d, J = 5.8 Hz, 1H), 7.39 |
| | 6-yl)oxy)cyclo- | (M + 1). | (d, J = 2.2 Hz, 1H), 7.31-7.28 (m, 1H), |
| | hexyl)carbamate | | 7.25-7.23 (m, 1H), 6.91-6.88 (m, 1H), |
| | | | 4.62-4.54 (m, 1H), 3.50-3.42 (m, 1H), |
| | | | 2.26-2.21 (m, 1H), 2.14-2.09 (m, 1H), |
| | | | 1.85-1.74 (m, 2H), 1.38-1.35 (m, 9H), |
| | | | 1.34-1.05 (m, 4H). |
| 357 | cis-3-((1-((2- | 351.2 | ¹H NMR (400 MHZ, DMSO-d₆) δ 9.44 |
| | methylpyrimidin-5- | (M + 1), | (s, 1H), 9.13 (s, 2H), 8.43 (d, J = 9.3 |
| | yl)amino)isoquinolin-6- | 353.2 | Hz, 1H), 7.90-7.89 (m, 1H), 7.36-7.34 |
| | yl)oxy)cyclohexan-1-ol | (M + 1). | (m, 1H), 7.29-7.26 (m, 1H), 7.18-7.17 |
| | | | (m, 1H), 4.77 (s, 1H), 4.57-4.49 (m, |
| | | | 1H), 3.63-3.55 (m, 1H), 2.60 (s, 3H), |
| | | | 2.38-2.33 (m, 1H), 2.12-2.07 (m, 1H), |
| | | | 1.88-1.83 (m, 1H), 1.78-1.73 (m, 1H), |
| | | | 1.41-1.07 (m, 4H). |

### Examples 358 and 359

### Synthesis of (1R,3S)-3-((1-((2-chloropyrimidin-5-yl)amino)isoquinolin-6-yl)oxy)cyclohexan-1-ol and (1 S,3R)-3-((1-((2-chloropyrimidin-5-yl)amino)isoquinolin-6-yl)oxy)cyclohexan-1-ol

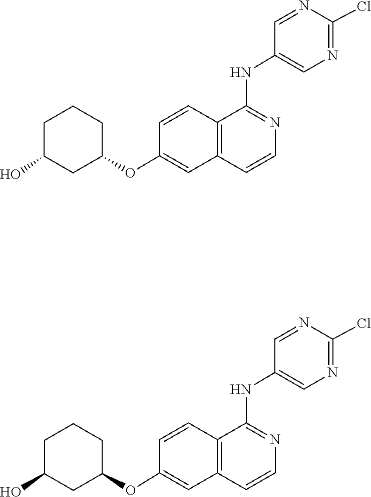 [SMILES: O[C@@H]1CCC[C@H](Oc2ccc3c(Nc4cnc(Cl)nc4)nccc3c2)C1 ; O[C@H]1CCC[C@@H](Oc2ccc3c(Nc4cnc(Cl)nc4)nccc3c2)C1]

Racemic cis-3-((1-((2-chloropyrimidin-5-yl)amino)isoquinolin-6-yl)oxy)cyclohexan-1-ol was synthesized as described in EXAMPLE 348. Resolution of the enantiomers by chiral SFC (ChiralPak IA, 10×250 mm, 5 μm column), eluting with 50% of methanol (containing 10 mM ammonium formate) in supercritical carbon dioxide, afforded the title compounds as single enantiomer as colorless solids. First eluting enantiomer (0.032 g, 11% yield): ¹H NMR (400 MHz, DMSO-d₆) δ 9.57 (s, 1H), 9.30 (s, 2H), 8.41 (d, J=9.3 Hz, 1H), 7.99 (d, J=5.8 Hz, 1H), 7.36 (d, J=2.5 Hz, 1H), 7.29 (dd, J=9.2, 2.5 Hz, 1H), 7.24 (d, J=5.9 Hz, 1H), 4.77-4.75 (m, 1H), 4.57-4.49 (m, 1H), 3.63-3.56 (m, 1H), 2.38-2.33 (m, 1H), 2.11-2.07 (m, 1H), 1.88-1.83 (m, 1H), 1.78-1.73 (m, 1H), 1.41-1.07 (m, 4H); MS (ES+) m/z 371.2 (M+1), 373.2 (M+1). Second eluting enantiomer (0.038 g, 13% yield): ¹H NMR (400 MHz, DMSO-d₆) δ 9.57 (s, 1H), 9.30 (s, 2H), 8.42-8.40 (m, 1H), 7.99 (d, J=5.8 Hz, 1H), 7.36 (d, J=2.6 Hz, 1H), 7.30-7.27 (m, 1H), 7.24 (d, J=6.1 Hz, 1H), 4.78-4.75 (m, 1H), 4.57-4.49 (m, 1H), 3.64-3.55 (m, 1H), 2.38-2.33 (m, 1H), 2.12-2.07 (m, 1H), 1.87-1.83 (m, 1H), 1.78-1.73 (m, 1H), 1.42-1.07 (m, 4H).; MS (ES+) m/z 371.2 (M+1), 373.2 (M+1).

### Example 360

### Synthesis of cis-N-(2-chloropyrimidin-5-yl)-6-(((1S,3R)-3-methoxycyclohexyl)oxy)isoquinolin-1-amine

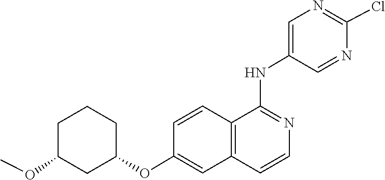 [SMILES: CO[C@@H]1CCC[C@H](Oc2ccc3c(Nc4cnc(Cl)nc4)nccc3c2)C1]

### Step 1. Preparation of 1-chloro-6-((cis-3-methoxycyclohexyl)oxy)isoquinoline

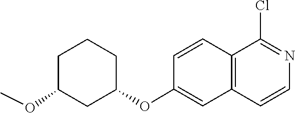 [SMILES: CO[C@@H]1CCC[C@H](Oc2ccc3c(Cl)nccc3c2)C1]

To a mixture of cis-3-((1-chloroisoquinolin-6-yl)oxy)cyclohexan-1-ol (0.050 g, 0.180 mmol) in N,N-dimethylformamide (1 mL) was added iodomethane (0.034 mL, 0.540 mmol) and sodium hydride (60% dispersion in mineral oil, 0.014 g, 0.360 mmol). The reaction mixture was stirred at ambient temperature for 1 h. The mixture was then diluted with ethyl acetate (30 mL) and washed with saturated sodium bicarbonate (30 mL) and brine (30 mL). The organic phase was dried over anhydrous sodium sulfate and filtered. The filtrate was concentrated in vacuo to give the title compound as a colorless solid (0.060 g, 82% yield): ¹H NMR (400 MHz, CDCl₃) δ 8.25 (d, J=9.2 Hz, 1H), 8.20 (d, J=5.7 Hz, 1H), 7.48 (d, J=5.8 Hz, 1H), 7.31 (d, J=2.5 Hz, 1H), 7.10 (d, J=2.5 Hz, 1H), 4.44-4.37 (m, 1H), 3.41 (s, 3H), 3.36-3.29 (m, 1H), 2.64-2.58 (m, 1H), 2.25-2.12 (m, 2H), 1.99-1.94 (m, 1H), 1.30-1.22 (m, 2H), 0.92-0.85 (m, 2H); MS (ES+) m/z 292.4 (M+1), 294.4 (M+1).

### Step 2. Preparation of rac-N-(2-chloropyrimidin-5-yl)-6-(((1 S,3R)-3-methoxycyclohexyl)oxy)isoquinolin-1-amine

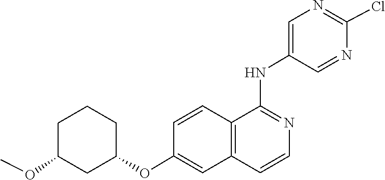 [SMILES: CO[C@@H]1CCC[C@H](Oc2ccc3c(Nc4cnc(Cl)nc4)nccc3c2)C1]

Following the procedure as described for EXAMPLE 348, Step 2, and making variations as required to replace cis-3-((1-chloroisoquinolin-6-yl)oxy)cyclohexan-1-ol with 1-chloro-6-((cis-3-methoxycyclohexyl)oxy)isoquinoline, the title compound was obtained as a colorless solid (0.026 g, 39% yield): ¹H NMR (400 MHz, DMSO-d₆) δ 9.57 (s, 1H), 9.30 (s, 2H), 8.41 (d, J=9.3 Hz, 1H), 7.99 (d, J=5.8 Hz, 1H), 7.38 (d, J=2.5 Hz, 1H), 7.29 (dd, J=9.2, 2.5 Hz, 1H), 7.25 (d, J=5.8 Hz, 1H), 4.59-4.51 (m, 1H), 3.33-3.29 (m, 1H), 3.29-3.27 (m, 3H), 2.15-2.02 (m, 2H), 1.83-1.78 (m, 1H), 1.39-1.22 (m, 4H), 1.13-1.03 (m, 1H); MS (ES+) m/z 385.0 (M+1), 387.0 (M+1).

### Example 361

### Synthesis of 1-(((1-((2-chloropyrimidin-5-yl)amino)isoquinolin-6-yl)oxy)methyl)cyclopropane-1-carboxamide

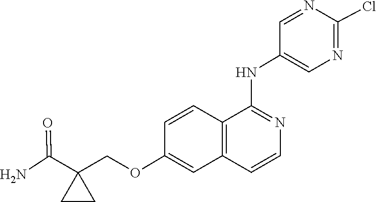 [SMILES: NC(=O)C1(COc2ccc3c(Nc4cnc(Cl)nc4)nccc3c2)CC1]

### Step 1. Preparation of ethyl 1-(((1-chloroisoquinolin-6-yl)oxy)methyl)cyclopropane-1-carboxylate

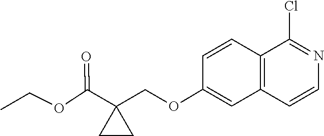 [SMILES: CCOC(=O)C1(COc2ccc3c(Cl)nccc3c2)CC1]

Following the procedure as described for EXAMPLE 348, Step 1, and making variations as required to replace cis-1,3-cyclohexanediol with ethyl 1-hydroxymethyl-cyclopropanecarboxylate, the title compound was obtained as a colorless solid (0.195 g, 34% yield): ¹H NMR (400 MHz, CDCl₃) δ 8.25 (d, J=9.2 Hz, 1H), 8.21 (d, J=5.7 Hz, 1H), 7.49 (d, J=5.6 Hz, 1H), 7.32 (dd, J=9.3, 2.5 Hz, 1H), 7.11 (d, J=2.4 Hz, 1H), 4.30 (s, 2H), 4.22-4.17 (m, 2H), 1.47 (q, J=3.6 Hz, 2H), 1.25 (t, J=7.2 Hz, 3H), 1.12 (q, J=3.6 Hz, 2H); MS (ES+) m/z 306.4 (M+1), 308.4 (M+1).

### Step 2. Preparation of 1-(((1-chloroisoquinolin-6-yl)oxy)methyl)cyclopropane-1-carboxylic acid

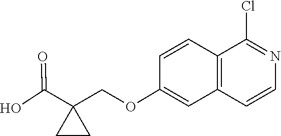 [SMILES: O=C(O)C1(COc2ccc3c(Cl)nccc3c2)CC1]

To the solution of ethyl 1-(((1-chloroisoquinolin-6-yl)oxy)methyl)cyclopropane-1-carboxylate (0.050 g, 0.164 mmol) in tetrahydrofuran (1 mL) was added a solution of lithium hydroxide monohydrate (0.012 g, 0.286 mmol) in water (1 mL). The reaction mixture was heated to 50° C. for 2 h. After cooling to ambient temperature, 1.0 M hydrochloric acid was added until pH ˜2 was reached. The mixture was diluted with water (30 mL) and extracted with ethyl acetate (30 mL). The organic phase was washed with brine (30 mL), dried over anhydrous sodium sulfate, and filtered. The filtrate was concentrated in vacuo to give title compound as a colorless solid (0.043 g, 81% yield): ¹H NMR (400 MHz, DMSO-d₆) δ 12.49 (s, 1H), 8.21 (d, J=5.7 Hz, 1H), 8.16 (d, J=9.1 Hz, 1H), 7.75 (dd, J=5.5, 0.3 Hz, 1H), 7.48-7.48 (m, 1H), 7.44 (dd, J=9.3, 2.4 Hz, 1H), 4.27 (s, 2H), 1.27-1.23 (m, 2H), 1.10-1.07 (m, 2H); MS (ES+) m/z 278.4 (M+1), 280.4 (M+1).

### Step 3. Preparation of 1-(((1-chloroisoquinolin-6-yl)oxy)methyl)cyclopropane-1-carboxamide

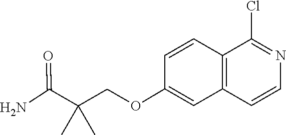 [SMILES: CC(C)(COc1ccc2c(Cl)nccc2c1)C(N)=O]

To a solution of 1-(((1-chloroisoquinolin-6-yl)oxy)methyl)cyclopropane-1-carboxylic acid (0.043 g, 0.153 mmol) in dichloromethane (2 mL) was added oxalyl chloride (0.015 mL, 0.175 mmol) followed by two drops of N,N-dimethylformamide. The mixture was stirred at ambient temperature for 4 h and was then concentrated in vacuo. To the obtained residue was added dichloromethane (2 mL), and to the mixture was then added a 7 N solution of ammonia in methanol (0.50 mL, 3.50 mmol). The reaction mixture was stirred for 20 minutes and then diluted with saturated aqueous ammonium chloride (20 mL). The resulting mixture was extracted with ethyl acetate (20 mL). The organic phase was washed with brine (30 mL), dried over anhydrous sodium sulfate, and filtered. The filtrate was concentrated in vacuo to give the title compound as a colorless solid (0.047 g, 87% yield): MS (ES+) m/z 277.4 (M+1), 279.4 (M+1).

### Step 4. Preparation of 1-(((1-((2-chloropyrimidin-5-yl)amino)isoquinolin-6-yl)oxy)methyl)cyclopropane-1-carboxamide

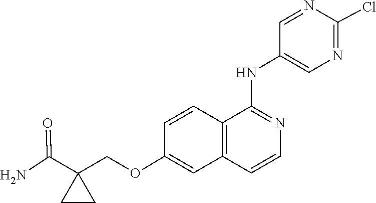 [SMILES: NC(=O)C1(COc2ccc3c(Nc4cnc(Cl)nc4)nccc3c2)CC1]

Following the procedure as described for EXAMPLE 348, Step 2, and making variations as required to replace rac-(1R,3S)-3-((1-((2-chloropyrimidin-5-yl)amino)isoquinolin-6-yl)oxy)cyclohexan-1-ol with 1-(((1-chloroisoquinolin-6-yl)oxy)methyl)cyclopropane-1-carboxamide, the title compound was obtained as a colorless solid (0.010 g, 15% yield): ¹H NMR (400 MHz, DMSO-d₆) δ 9.60 (s, 1H), 9.32 (s, 2H), 8.44 (d, J=9.3 Hz, 1H), 8.00 (d, J=5.8 Hz, 1H), 7.36-7.33 (m, 1H), 7.28 (d, J=2.5 Hz, 1H), 7.23 (d, J=5.8 Hz, 1H), 7.18 (s, 1H), 7.05 (s, 1H), 4.27 (s, 2H), 1.16-1.14 (m, 2H), 0.90-0.86 (m, 2H); MS (ES+) m/z 370.0 (M+1), 372.0 (M+1).

### Example 362

### Synthesis of N-(6-chloropyridin-3-yl)-6-((1-(pyridin-4-ylmethoxy)cyclopropyl)methoxy)isoquinolin-1-amine

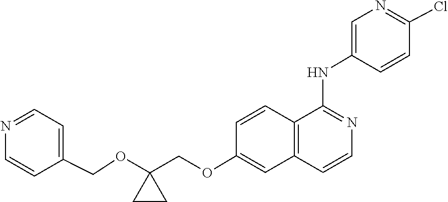 [SMILES: Clc1ccc(Nc2nccc3cc(OCC4(OCc5ccncc5)CC4)ccc23)cn1]

### Step 1. Preparation of methyl 1-(pyridin-4-ylmethoxy)cyclopropane-1-carboxylate

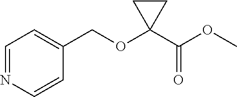

To a solution of methyl 1-hydroxy-1-cyclopropanecarboxylate (0.200 g, 1.72 mmol) in N,N-dimethylformamide (8.6 mL) was added sodium hydride (60% dispersion in mineral oil, 0.207 g, 5.17 mmol) at 0° C., and the mixture was stirred at this temperature for 30 minutes. To the mixture was then added 4-(bromomethyl)pyridine hydrobromide (0.479 g, 1.89 mmol). The reaction mixture was allowed to warm to ambient temperature and stirred for 5 h. The reaction mixture was quenched by addition of saturated ammonium chloride solution (30 mL) and extracted with ethyl acetate (3×50 mL). The combined organic layers were dried over anhydrous sodium sulfate and filtered. The filtrate was concentrated in vacuo to afford a residue which was purified by silica gel column chromatography, eluting with a gradient of 0 to 100% of ethyl acetate in heptane and 0 to 10% of methanol in dichloromethane, to provide the title compound as a colorless oil: MS (ES+) m/z 208.0 (M+1).

### Step 2. Preparation of (1-(pyridin-4-ylmethoxy)cyclopropyl)methanol

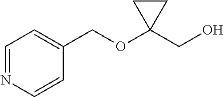 [SMILES: OCC1(OCc2ccncc2)CC1]

Following the procedure as described for EXAMPLE 151, Step 1, and making variations as required to replace 1-ethynylcyclopropane-1-carboxylic acid with methyl 1-(pyridin-4-ylmethoxy)cyclopropane-1-carboxylate, the title compound was obtained as a colorless oil (0.090 g, 36% yield): ¹H NMR (400 MHz, CDCl₃) δ 8.53 (d, J=6.1 Hz, 2H), 7.23 (d, J=6.0 Hz, 2H), 4.67 (s, 2H), 3.77 (s, 2H), 0.97 (t, J=6.2 Hz, 2H), 0.71-0.67 (m, 2H), OH not observed.

### Step 3. Preparation of N-(6-chloropyridin-3-yl)-6-((1-(pyridin-4-ylmethoxy)cyclopropyl)methoxy)isoquinolin-1-amine

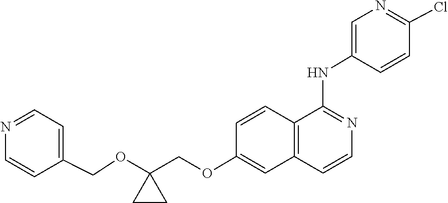 [SMILES: Clc1ccc(Nc2nccc3cc(OCC4(OCc5ccncc5)CC4)ccc23)cn1]

Following the procedure as described for EXAMPLE 279, Step 2, and making variations as required to replace (1-(difluoromethyl)-1H-pyrazol-4-yl)methanol with (1-(pyridin-4-ylmethoxy)cyclopropyl)methanol, the title compound was obtained as a yellow solid (0.057 g, 74% yield): ¹H NMR (400 MHz, DMSO-d₆) δ10.10 (br s, 1H), 8.79 (d, J=2.3 Hz, 1H), 8.71-8.70 (m, 2H), 8.51 (d, J=9.3 Hz, 1H), 8.29-8.27 (m, 1H), 7.82-7.80 (m, 1H), 7.75-7.72 (m, 2H), 7.59 (d, J=8.5 Hz, 1H), 7.42-7.38 (m, 2H), 7.21 (d, J=6.5 Hz, 1H), 4.95 (s, 2H), 4.39 (s, 2H), 1.09 (t, J=6.0 Hz, 2H), 0.88 (t, J=6.1 Hz, 2H); MS (ES+) m/z 433.0 (M+1), 435.0 (M+1).

### Example 363

### Synthesis of N-(6-chloropyridin-3-yl)-6-((3-fluorotetrahydrofuran-3-yl)methoxy)isoquinolin-1-amine

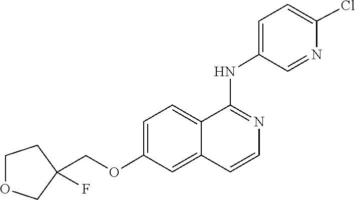 [SMILES: FC1(COc2ccc3c(Nc4ccc(Cl)nc4)nccc3c2)CCOC1]

### Step 1. Preparation of (3-fluorotetrahydrofuran— 3-yl)methyl-4-methylbenzenesulfonate

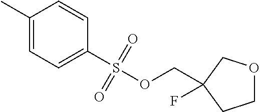 [SMILES: Cc1ccc(S(=O)(=O)OCC2(F)CCOC2)cc1]

To a mixture of (3-fluorotetrahydrofuran-3-yl)methanol (0.200 g, 1.66 mmol), triethylamine (0.303 g, 3.00 mmol, 0.417 mL), and 4-dimethylaminopyridine (0.0203 g, 0.167 mmol) in dichloromethane (10 mL) was added 4-methylbenzenesulfonyl chloride (0.333 g, 1.75 mmol) and the mixture was stirred at ambient temperature for 2 h. The reaction mixture was diluted with water (30 mL) and extracted with ethyl acetate (3×30 mL). The combined organic phase was washed with brine (3×30 mL), dried over anhydrous sodium sulfate, and filtered.

Concentration of the filtrate under reduced pressure and purification of the residue by silica gel column chromatography, eluting with a gradient of 0 to 25% of ethyl acetate in petroleum ether, provided the title compound as a colorless oil (0.200 g, 44% yield): ¹H NMR (400 MHz, DMSO-d₆) δ 7.81 (d, J=8.2 Hz, 2H), 7.50 (d, J=8.2 Hz, 2H), 4.39-4.33 (m, 2H), 3.86-3.73 (m, 3H), 3.68-3.57 (m, 1H), 2.43 (s, 3H), 2.09-1.97 (m, 2H); ¹⁹F NMR (376 MHz, DMSO-d₆) δ−151.8 (s).

### Step 1. Preparation of 1-chloro-6-((3-fluorotetrahydrofuran-3-yl)methoxy)isoquinoline

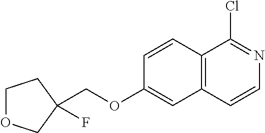 [SMILES: FC1(COc2ccc3c(Cl)nccc3c2)CCOC1]

To a solution of 1-chloroisoquinolin-6-ol (0.0900 g, 0.501 mmol) in N,N-dimethyl formamide (8 mL) was added (3-fluorotetrahydrofuran-3-yl)methyl 4-methylbenzenesulfonate (0.165 g, 0.602 mmol) and potassium carbonate (0.139 g, 1.00 mmol) and the mixture was stirred at 80° C. for 16 h. After cooling to ambient temperature, the reaction mixture was diluted with water (30 mL) and extracted with ethyl acetate (3×30 mL). The combined organic phase was washed with brine (3×30 mL), dried over anhydrous sodium sulfate, and filtered. Concentration of the filtrate under reduced pressure and purification of the obtained residue by silica gel column chromatography, eluting with a gradient of 0 to 25% of ethyl acetate in petroleum ether, afforded the title compound as a colorless solid (0.220 g, quantitative yield): ¹H NMR (400 MHz, DMSO-d₆) δ8.23-8.18 (m, 2H), 7.76 (d, J=5.6 Hz, 1H), 7.55 (d, J=2.4 Hz, 1H), 7.47 (dd, J=9.2, 2.4 Hz, 1H), 4.59-4.45 (m, 2H), 4.00-3.82 (m, 4H), 2.30-2.18 (m, 2H); ¹⁹F NMR (376 MHz, DMSO-d₆) δ−151.1 (s).

### Step 3. Preparation of N-(6-chloropyridin-3-yl)-6-((3-fluorotetrahydrofuran-3-yl)methoxy)isoquinolin-1-amine

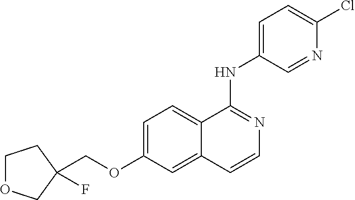 [SMILES: FC1(COc2ccc3c(Nc4ccc(Cl)nc4)nccc3c2)CCOC1]

To a solution of 1-chloro-6-((3-fluorotetrahydrofuran-3-yl)methoxy)isoquinoline (0.200 g, 0.710 mmol) and 6-chloropyridin-3-amine (0.110 g, 0.852 mmol) in isopropyl alcohol (15 mL) was added a 4 M solution of hydrogen chloride in dioxane (1.50 mL, 4.0 mmol). The reaction mixture was stirred at 70° C. for 16 h. After cooling to ambient temperature, the reaction mixture was diluted with saturated with sodium bicarbonate (30 mL) and extracted with ethyl acetate (3×30 mL). The combined organic phase was washed with brine (3×30 mL), dried over anhydrous sodium sulfate, and filtered. The filtrate was concentrated under reduced pressure and the obtained residue was purified by silica gel column chromatography, eluting with a gradient of 0 to 49% of ethyl acetate in petroleum ether. The residue was then purified by reverse-phase preparative HPLC (Phenomenex Luna C 18 150 mm×25 mm, 10 μm column), eluting with a gradient of 13 to 43% of acetonitrile in water (containing formic acid), to give the title compound as a colorless solid (0.075 g, 28% yield): ¹H NMR (400 MHz, DMSO-d₆) δ9.98-9.92 (m, 1H), 8.85-8.82 (m, 1H), 8.53 (d, J=9.0 Hz, 1H), 8.34-8.32 (m, 1H), 7.88-7.87 (m, 1H), 7.55 (d, J=8.6 Hz, 1H), 7.42 (d, J=9.5 Hz, 2H), 7.23 (d, J=6.1 Hz, 1H), 4.59-4.45 (m, 2H), 4.06-3.84 (m, 4H), 2.30-2.19 (m, 2H); MS (ES+) m/z 374.2 (M+1), 376.2 (M+1).

### Examples 364 and 365

### Synthesis of (R)—N-(6-chloropyridin-3-yl)-6-((3-fluorotetrahydrofuran-3-yl)methoxy)isoquinolin-1-amine and (S)—N-(6-chloropyridin-3-yl)-6-((3-fluorotetrahydrofuran-3-yl)methoxy)isoquinolin-1-amine

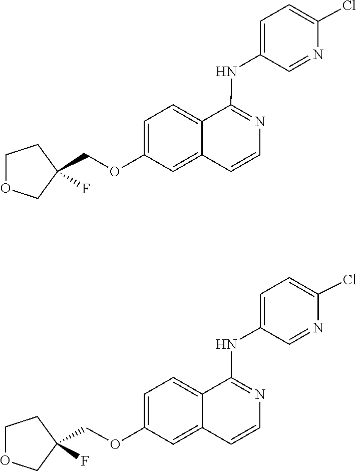 [SMILES: F[C@@]1(COc2ccc3c(Nc4ccc(Cl)nc4)nccc3c2)CCOC1 ; F[C@]1(COc2ccc3c(Nc4ccc(Cl)nc4)nccc3c2)CCOC1]

Racemic N-(6-chloropyridin-3-yl)-6-((3-fluorotetrahydrofuran-3-yl)methoxy)isoquinolin-1-amine was synthesized as described in EXAMPLE 363. Resolution of the enantiomers by chiral SFC (ChiralPak AD 250×30 mm, 10 μm column), eluting with 60% of a mixture of methanol and acetonitrile (containing 0.1% of ammonium hydroxide) in supercritical carbon dioxide, afforded the title compounds as single enantiomers as off-white solids. First eluting enantiomer (0.036 g, 48% yield): ¹H NMR (400 MHz, DMSO-d₆) δ 9.49 (s, 1H), 8.90-8.81 (m, 1H), 8.49-8.39 (m, 2H), 7.96-7.91 (m, 1H), 7.47 (d, J=9.2 Hz, 1H), 7.36-7.33 (m, 2H), 7.18 (d, J=5.6 Hz, 1H), 4.56-4.42 (m, 2H), 4.05-3.83 (m, 4H), 2.30-2.19 (m, 2H); ¹⁹F NMR (376 MHz, DMSO-d₆) δ−151.1 (s); MS (ES+) m/z 374.1 (M+1), 376.1 (M+1). Second eluting enantiomer (0.037 g, 49% yield): ¹H NMR (400 MHz, DMSO-d₆) δ 9.45 (s, 1H), 8.87 (d, J=2.4 Hz, 1H), 8.49-8.39 (m, 2H), 7.96 (d, J=5.6 Hz, 1H), 7.46 (d, J=8.8 Hz, 1H), 7.35-7.33 (m, 2H), 7.18 (d, J=5.6 Hz, 1H), 4.56-4.42 (m, 2H), 4.05-3.83 (m, 4H), 2.30-2.19 (m, 2H); ¹⁹F NMR (376 MHz, DMSO-d₆) δ−151.1 (s); MS (ES+) m/z 374.1 (M+1), 376.0 (M+1).

Examples 366 and 367 Synthesis of cis-3-(((1-((2-chloropyrimidin-5-yl)amino)isoquinolin-6-yl)oxy)methyl)-1-iminohexahydro-1λ⁶-thiopyran 1-oxide and trans-3-(((1-((2-chloropyrimidin-5-yl)amino)isoquinolin-6-yl)oxy)methyl)-1-iminohexahydro-1λ⁶-thiopyran 1-oxide

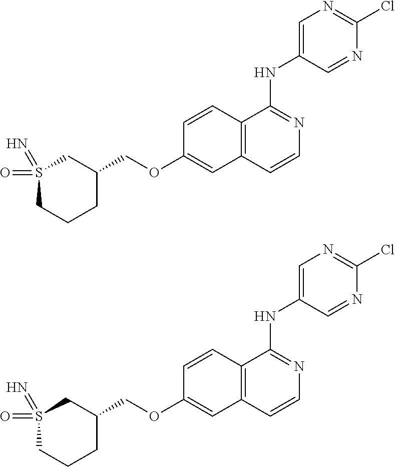 [SMILES: N=[S@]1(=O)CCC[C@@H](COc2ccc3c(Nc4cnc(Cl)nc4)nccc3c2)C1 ; N=[S@@]1(=O)CCC[C@@H](COc2ccc3c(Nc4cnc(Cl)nc4)nccc3c2)C1]

### Step 1. Preparation of 1-chloro-6-((tetrahydro-2H-thiopyran-3-yl)methoxy)isoquinoline

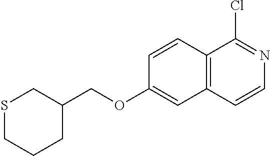 [SMILES: Clc1nccc2cc(OCC3CCCSC3)ccc12]

Following the procedure as described for EXAMPLE 208, Step 1 and making variations as required to replace 3-(hydroxymethyl)oxetane-3-carbonitrile with (tetrahydro-2H-thiopyran-3-yl)methanol, the title compound was obtained as a colorless solid (0.199 g, 61% yield): MS (ES+) m/z 294.6 (M+1), 296.6 (M+1).

### Step 2. Preparation of N-(2-chloropyrimidin-5-yl)-6-((tetrahydro-2H-thiopyran-3-yl)methoxy)isoquinolin-1-amine

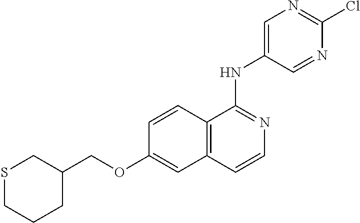 [SMILES: Clc1ncc(Nc2nccc3cc(OCC4CCCSC4)ccc23)cn1]

Following the procedure as described for EXAMPLE 207, Step 3 and making variations as required to replace 1-(((1-chloroisoquinolin-6-yl)oxy)methyl)cyclopropane-1-carbonitrile with 1-chloro-6-((tetrahydro-2H-thiopyran-3-yl)methoxy)isoquinoline, the title compound was obtained as a colorless solid (0.138 g, 54% yield): MS (ES+) m/z 387.6 (M+1), 389.6 (M+1).

### Step 3. Preparation of cis-3-(((1-((2-chloropyrimidin-5-yl)amino)isoquinolin-6-yl)oxy)methyl)-1-iminohexahydro-1λ⁶-thiopyran 1-oxide and trans-3-(((1-((2-chloropyrimidin-5-yl)amino)isoquinolin-6-yl)oxy)methyl)-1-iminohexahydro-1λ⁶-thiopyran 1-oxide

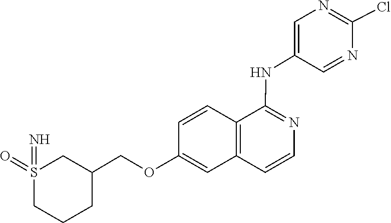 [SMILES: N=S1(=O)CCCC(COc2ccc3c(Nc4cnc(Cl)nc4)nccc3c2)C1]

To a mixture of iodobenzene diacetate (0.281 g, 0.872 mmol), ammonium carbamate (0.055 g, 0.698 mmol) and N-(2-chloropyrimidin-5-yl)-6-((tetrahydro-2H-thiopyran-3-yl)methoxy)isoquinolin-1-amine (0.135 g, 0.349 mmol) was added methanol (2.5 mL). The reaction mixture was stirred for 72 h at ambient temperature. To it was then added iodobenzene diacetate (0.281 g, 0.872 mmol), ammonium carbamate (0.055 g, 0.698 mmol), and methanol (1 mL), and the reaction mixture was heated to 65° C. for 3 h. After cooling to ambient temperature, the reaction mixture was concentrated in vacuo. The residue was purified by silica gel column chromatography, eluting with a gradient of 3 to 20% of methanol in dichloromethane. The residue was then purified by reverse-phase preparative HPLC (Phenomenex Gemini-NX C18 150 mm×30 mm, 5 μm column), eluting with a gradient of 10 to 40% of acetonitrile in water (containing 0.5% of formic acid), to provide the title compounds as pure diastereoisomers as colorless solids. First eluting diastereoisomer (0.0135 g, 9% yield): ¹H NMR (400 MHz, DMSO-d₆) δ9.58 (s, 1H), 9.29 (s, 2H), 8.43 (d, J=9.0 Hz, 1H), 8.00 (d, J=5.8 Hz, 1H), 7.35-7.31 (m, 2H), 7.24 (d, J=5.8 Hz, 1H), 4.11-4.08 (m, 1H), 4.04-4.00 (m, 1H), 3.52 (m, 1H), 3.23-3.19 (m, 1H), 3.02-2.94 (m, 3H), 2.48-2.41 (m, 1H), 2.05-1.99 (m, 1H), 1.93-1.83 (m, 2H), 1.46-1.35 (m, 1H); MS (ES+) m/z 418.2 (M+1), 420.0 (M+1). Second eluting diastereoisomer, isolated as formate salt (0.0116 g, 8% yield): ¹H NMR (400 MHz, DMSO-d₆) δ9.59 (s, 1H), 9.29 (s, 2H), 8.43 (d, J=9.1 Hz, 1H), 8.21 (s, 0.8H), 8.00 (d, J=5.8 Hz, 1H), 7.35-7.31 (m, 2H), 7.25 (d, J=5.8 Hz, 1H), 4.13 (dd, J=9.5, 5.6 Hz, 1H), 4.04 (dd, J=9.4, 7.0 Hz, 1H), 3.22-3.19 (m, 1H), 2.98-2.90 (m, 4H), 2.45-2.42 (m, 1H), 2.07-2.00 (m, 1H), 1.92-1.82 (m, 2H), 1.44-1.34 (m, 1H), COOH not observed; MS (ES+) m/z 418.0 (M+1), 420.0 (M+1).

### Example 368

### Synthesis of N-(6-chloropyridin-3-yl)-6-(isoxazol-4-ylmethoxy)isoquinolin-1-amine

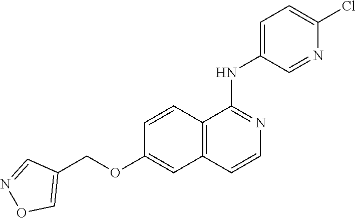 [SMILES: Clc1ccc(Nc2nccc3cc(OCc4cnoc4)ccc23)cn1]

Following the procedure as described for EXAMPLE 241, Step 2 and making variations as required to replace 1-((2-methylpyrimidin-5-yl)amino)isoquinolin-6-ol with 1-((6-chloropyridin-3-yl)amino)isoquinolin-6-ol, the title compound was obtained as a colorless solid (0.0098 g, 14% yield): ¹H NMR (400 MHz, DMSO-d₆) δ9.40 (s, 1H), 9.17 (s, 1H), 8.88 (d, J=2.8 Hz, 1H), 8.83 (s, 1H), 8.45 (d, J=9.2 Hz, 1H), 8.42 (dd, J=8.8, 2.8 Hz, 1H), 7.98 (d, J=5.7 Hz, 1H), 7.45 (d, J=8.7 Hz, 1H), 7.43 (d, J=2.5 Hz, 1H), 7.32 (dd, J=9.2, 2.6 Hz, 1H), 7.21 (d, J=5.8 Hz, 1H), 5.20 (s, 2H); MS (ES+) m/z 352.8 (M+1), 354.8 (M+1).

### Example 369

### Synthesis of 1-(((1-((2-chloropyrimidin-5-yl)amino)isoquinolin-6-yl)oxy)methyl)cyclobutane-1-carbonitrile

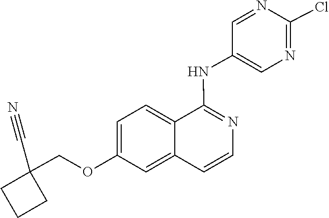 [SMILES: N#CC1(COc2ccc3c(Nc4cnc(Cl)nc4)nccc3c2)CCC1]

### Step 1. Preparation of 1-(((1-chloroisoquinolin-6-yl)oxy)methyl)cyclobutane-1-carbonitrile

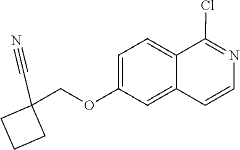 [SMILES: N#CC1(COc2ccc3c(Cl)nccc3c2)CCC1]

Following the procedure as described for EXAMPLE 208, Step 1 and making variations as required to replace 3-(hydroxymethyl)oxetane-3-carbonitrile with (1-(hydroxymethyl)cyclobutane-1-carbonitrile, the title compound was obtained as a colorless solid (0.470 g, 63% yield): MS (ES+) m/z 273.6 (M+1), 275.6 (M+1).

### Step 2. Preparation of 1-(((1-((2-chloropyrimidin-5-yl)amino)isoquinolin-6-yl)oxy)methyl)cyclobutane-1-carbonitrile

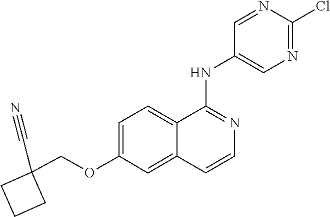 [SMILES: N#CC1(COc2ccc3c(Nc4cnc(Cl)nc4)nccc3c2)CCC1]

Following the procedure as described for EXAMPLE 208, Step 2 and making variations as required to replace 3-(((1-chloroisoquinolin-6-yl)oxy)methyl)oxetane-3-carbonitrile with 1-(((1-chloroisoquinolin-6-yl)oxy)methyl)cyclobutane-1-carbonitrile, the title compound was obtained as a colorless solid (0.0660 g, 25% yield): ¹H NMR (400 MHz, DMSO-d₆) δ9.61 (s, 1H), 9.30 (s, 2H), 8.46 (d, J=10.1 Hz, 1H), 8.02 (d, J=5.8 Hz, 1H), 7.39 (m, 2H), 7.24 (d, J=5.8 Hz, 1H), 4.45 (s, 2H), 2.59-2.53 (m, 2H), 2.35-2.28 (m, 2H), 2.21-2.07 (m, 2H); MS (ES+) m/z 366.0 (M+1), 368.0 (M+1).

### Example 370

### Synthesis of N-(2-methoxypyrimidin-5-yl)-6-((3-methyloxetan-3-yl)methoxy)isoquinolin-1-amine

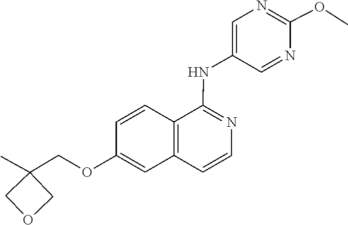

### Step 1. Preparation of 6-fluoro-N-(2-methoxypyrimidin-5-yl)isoquinolin-1-amine

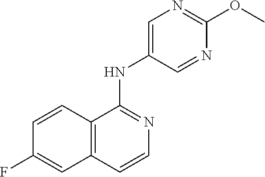 [SMILES: COc1ncc(Nc2nccc3cc(F)ccc23)cn1]

Following the procedure as described for EXAMPLE 76, Step 1, and making variations as required to replace 5-amino-2-chloropyridine with 2-methoxypyrimidin-5-amine, the title compound was obtained as a yellow solid (1.83 g, 58% yield): ¹H NMR (400 MHz, DMSO-d₆) δ 9.39 (s, 1H), 8.97 (s, 2H), 8.57 (dd, J=9.2, 5.5 Hz, 1H), 7.97 (d, J=5.4 Hz, 1H), 7.66 (dd, J=10.0, 2.6 Hz, 1H), 7.57 (td, J=8.9, 2.7 Hz, 1H), 7.20 (d, J=5.8 Hz, 1H), 3.92 (s, 3H); MS (ES+) m/z 271.5 (M+1).

### Step 2. Preparation of 6-fluoro-N-(2-methoxypyrimidin-5-yl)-N-((2-(trimethylsilyl)ethoxy)methyl)isoquinolin-1-amine

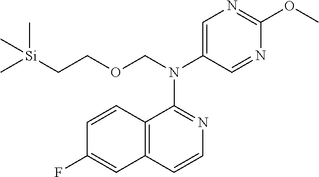

Following the procedure as described for EXAMPLE 76, Step 2, and making variations as required to replace N-(6-chloropyridin-3-yl)-6-fluoroisoquinolin-1-amine with 6-fluoro-N-(2-methoxypyrimidin-5-yl)isoquinolin-1-amine, the title compound was obtained as a yellow solid (1.56 g, 58% yield): ¹H NMR (400 MHz, CDCl₃) δ8.29 (d, J=5.6 Hz, 1H), 8.24 (s, 2H), 7.84 (dd, J=9.3, 5.5 Hz, 1H), 7.44-7.41 (m, 2H), 7.18 (ddd, J=9.2, 8.3, 2.5 Hz, 1H), 5.36 (s, 2H), 3.95 (s, 3H), 3.60-3.55 (m, 2H), 0.92-0.85 (m, 2H), −0.10 (s, 9H); MS (ES+) m/z 401.2 (M+1).

### Step 3. Preparation of N-(2-methoxypyrimidin-5-yl)-6-((3-methyloxetan-3-yl)methoxy)isoquinolin-1-amine

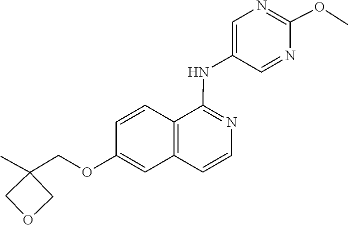

To a solution of 6-fluoro-N-(2-methoxypyrimidin-5-yl)-N-((2-(trimethylsilyl)ethoxy)methyl)isoquinolin-1-amine (0.0750 g, 0.165 mmol) in N,N-dimethylformamide (1.5 mL) was added 3-methyl-3-oxetanemethanol (0.020 g, 0.198 mmol) and potassium tert-butoxide (1 M solution in tetrahydrofuran, 0.247 mL, 0.247 mmol), and the resulting mixture was heated to 80° C. for 1 h. After cooling to ambient temperature, the mixture was concentrated in vacuo. To the obtained residue was added dichloromethane (3 mL) and trifluoroacetic acid (0.252 mL, 3.30 mmol), and the reaction mixture was stirred at ambient temperature for 6 h. The reaction mixture was concentrated in vacuo to provide a residue which was purified by silica gel column chromatography, eluting with a gradient of 0 to 100% of ethyl acetate in heptane. Further purification by reverse phase column chromatography, eluting with a gradient of 5 to 35% of acetonitrile in water (containing 0.5% of formic acid), provided the title compound as a colorless solid (0.003 g, 4% yield): ¹H NMR (400 MHz, DMSO-d₆) δ 9.24 (s, 1H), 8.99 (s, 2H), 8.41 (d, J=8.9 Hz, 1H), 7.89 (d, J=5.7 Hz, 1H), 7.32 (d, J=9.0 Hz, 2H), 7.12 (d, J=5.8 Hz, 1H), 4.55 (d, J=5.8 Hz, 2H), 4.35 (d, J=5.8 Hz, 2H), 4.22 (s, 2H), 3.91 (s, 3H), 1.41 (s, 3H); MS (ES+) m/z 353.0 (M+1).

### Examples 371-373

In a similar manner as described in EXAMPLE 370, utilizing the appropriately substituted starting materials and intermediates, the following compounds were prepared:

| | | MS | |
|---|---|---|---|
| Compound | | (ES+) | |
| No. | Name | m/z | NMR |
| 371 | N-(2-methoxypyrimidin- | 363.0 | ¹H NMR (400 MHZ, DMSO-d₆) δ 9.22 |
| | 5-yl)-6-((1-methyl-1H- | (M + 1). | (s, 1H), 8.98 (s, 2H), 8.39 (s, 1H), |
| | pyrazol-4- | | 7.88 (d, J = 5.8 Hz, 1H), 7.86 (s, 1H), |
| | yl)methoxy)isoquinolin- | | 7.57 (s, 1H), 7.37 (s, 1H), 7.25 (dd, |
| | 1-amine | | J = 9.1, 2.4 Hz, 1H), 7.12 (d, J = 5.8 Hz, |
| | | | 1H), 5.10 (s, 2H), 3.91 (s, 3H), 3.83 |
| | | | (s, 3H). |
| 372 | 6-(cyclopropylmethoxy)- | 323.0 | ¹H NMR (400 MHZ, DMSO-d₆) δ 9.22 |
| | N-(2-methoxypyrimidin- | (M + 1). | (d, J = 0.4 Hz, 1H), 8.98 (s, 2H), 8.37 |
| | 5-yl)isoquinolin-1- | | (d, J = 9.1 Hz, 1H), 7.87 (d, J = 5.6 |
| | amine | | Hz, 1H), 7.26 (d, J = 9.1 Hz, 1H), 7.22 |
| | | | (s, 1H), 7.09 (d, J = 5.9 Hz, 1H), 3.97 |
| | | | (d, J = 7.1 Hz, 2H), 3.90 (s, 3H), 1.30 |
| | | | (s, 1H), 0.61 (d, J = 7.7 Hz, 2H), 0.38 |
| | | | (d, J = 4.8 Hz, 2H). |
| 373 | 1-(((1-((2- | 348.0 | ¹H NMR (400 MHZ, DMSO-d₆) δ 8.90 |
| | methoxypyrimidin-5- | (M + 1). | (s, 2H), 8.55 (d, J = 9.3 Hz, 1H), 7.71 |
| | yl)amino)isoquinolin-6- | | (d, J = 6.6 Hz, 1H), 7.51 (d, J = 9.3 |
| | yl)oxy)methyl)cyclopro | | Hz, 1H), 7.42 (s, 1H), 7.22 (d, J = 6.1 |
| | pane-1-carbonitrile | | Hz, 1H), 4.27 (s, 2H), 3.97 (s, 3H), |
| | | | 1.45 (q, J = 3.7 Hz, 2H), 1.24 (dt, J = |
| | | | 3.6, 1.3 Hz, 2H), NH not observed. |

### Example 374

### Synthesis of 2-chloro-5-((6-((1-methyl-1H-pyrazol-4-yl)methoxy)isoquinolin-1-yl)amino)pyridin-3-ol

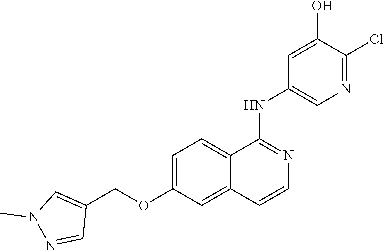 [SMILES: Cn1cc(COc2ccc3c(Nc4cnc(Cl)c(O)c4)nccc3c2)cn1]

### Step 1. Preparation of 5-amino-2-chloropyridin-3-ol

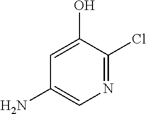

To a solution of 6-chloro-5-methoxy-pyridin-3-amine (0.300 g, 1.89 mmol) in dichloromethane (10 mL) was added a solution of tribromoborane (7.11 g, 28.4 mmol) in dichloromethane (15 mL) dropwise at 10° C. The reaction mixture was allowed to warm to ambient temperature and stirred for 12 h. After cooling to 0° C., the reaction was quenched by addition of water (10 mL) and the reaction mixture was adjusted to pH=7-8 with sodium bicarbonate (10 g). The mixture was extracted with ethyl acetate (3×50 mL). The combined organic phase was washed with brine (10 mL), dried over anhydrous sodium sulfate, and filtered. The filtrate was concentrated in vacuo to give the title compound as a yellowish solid (0.274 g, quantitative yield): ¹H NMR (400 MHz, DMSO-d₆) δ 10.05 (s, 1H), 7.20 (d, J=2.4 Hz, 1H), 6.56 (d, J=2.4 Hz, 1H), 5.68-5.07 (m, 2H).

### Step 2. Preparation of 1-chloro-6-((1-methyl-1H-pyrazol-4-yl)methoxy)isoquinoline

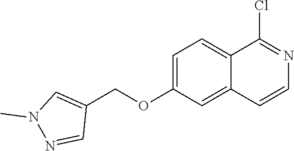 [SMILES: Cn1cc(COc2ccc3c(Cl)nccc3c2)cn1]

Following the procedure as described for EXAMPLE 188, Step 1, and making variations as required to replace 3-(chloromethyl)-1-ethyl-1H-pyrazole with 4-(chloromethyl)-1-methyl-1H-pyrazole, the title compound was obtained as a yellow solid (1.70 g, 56% yield): ¹H NMR (400 MHz, DMSO-d₆) δ 8.21 (d, J=5.6 Hz, 1H), 8.15 (d, J=9.2 Hz, 1H), 7.87 (s, 1H), 7.77 (d, J=5.6 Hz, 1H), 7.62-7.57 (m, 2H), 7.40 (dd, J=2.4, 9.2 Hz, 1H), 5.14 (s, 2H), 3.83 (s, 3H).

### Step 3. Preparation of 2-chloro-5-((6-((1-methyl-1H-pyrazol-4-yl)methoxy)isoquinolin-1-yl)amino)pyridin-3-ol

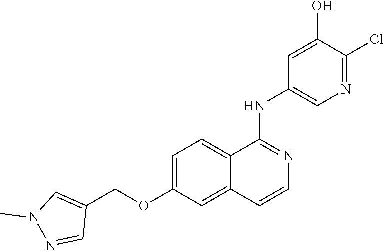 [SMILES: Cn1cc(COc2ccc3c(Nc4cnc(Cl)c(O)c4)nccc3c2)cn1]

A mixture of 1-chloro-6-((1-methyl-1H-pyrazol-4-yl)methoxy)isoquinoline (0.200 g, 0.731 mmol), 5-amino-2-chloropyridin-3-ol (0.111 g, 0.767 mmol), [(2-di-tert-butylphosphino-2′,4′,6′-triisopropyl-1,1′-biphenyl)-2-(2′-amino-1,1′-biphenyl)] palladium(II) methanesulfonate (0.058 g, 0.0731 mmol), and cesium carbonate (0.714 g, 2.19 mmol) in 2-methylbutan-2-ol (10 mL) was stirred at 70° C. for 12 h. After cooling to ambient temperature, the reaction mixture was poured into water (20 mL). The mixture was extracted with ethyl acetate (3×50 mL). The combined organic layers were washed with brine (30 mL), dried over anhydrous sodium sulfate, and filtered. The filtrate was concentrated in vacuo and the obtained residue was purified by reverse-phase preparative HPLC (Phenomenex Gemini-NX C18 75 mm×30 mm, 3 μm column), eluting with a gradient of 8% to 38% of acetonitrile in water (containing 0.225% of formic acid), to provide the title compound as a yellow solid (0.051 g, 17% yield): ¹H NMR (400 MHz, DMSO-d₆) δ 10.55 (br s, 1H), 9.24 (s, 1H), 8.43 (d, J=9.2 Hz, 1H), 8.32 (d, J=2.0 Hz, 1H), 8.19 (d, J=2.0 Hz, 1H), 7.97 (d, J=5.6 Hz, 1H), 7.86 (s, 1H), 7.57 (s, 1H), 7.38 (d, J=2.4 Hz, 1H), 7.29-7.22 (m, 1H), 7.18 (d, J=6.0 Hz, 1H), 5.10 (s, 2H), 3.83 (s, 3H); MS (ES+) m/z 382.1 (M+1), 384.1 (M+1).

### Examples 375 and 376

### Synthesis of N-((1R,3S)-3-((1-((2-chloropyrimidin-5-yl)amino)isoquinolin-6-yl)oxy)cyclohexyl)-2,2,2-trifluoroacetamide and N-((1R,3S)-3-((1-((2-chloropyrimidin-5-yl)amino)isoquinolin-6-yl)oxy)cyclohexyl)acetamide

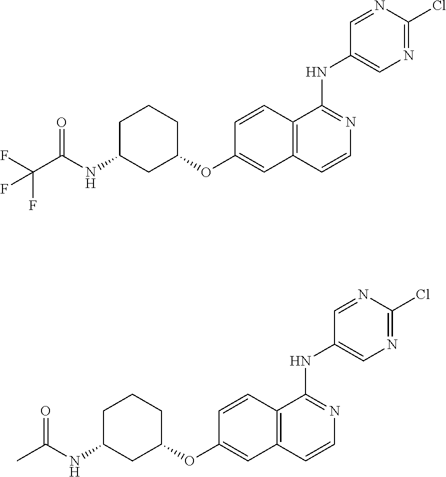 [SMILES: O=C(N[C@@H]1CCC[C@H](Oc2ccc3c(Nc4cnc(Cl)nc4)nccc3c2)C1)C(F)(F)F ; CC(=O)N[C@@H]1CCC[C@H](Oc2ccc3c(Nc4cnc(Cl)nc4)nccc3c2)C1]

### Step 1. Preparation of tert-butyl ((1R,3S)-3-((1-((2-chloropyrimidin-5-yl)amino)isoquinolin-6-yl)oxy)cyclohexyl)carbamate

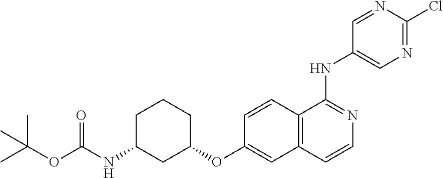 [SMILES: CC(C)(C)OC(=O)N[C@@H]1CCC[C@H](Oc2ccc3c(Nc4cnc(Cl)nc4)nccc3c2)C1]

Following the procedure as described for EXAMPLE 348, and making variations as required to replace cis-1,3-cyclohexanediol with tert-butyl ((1R,3S)-3-hydroxycyclohexyl)carbamate, the title compound was obtained as a colorless solid (0.040 g, 25% yield): ¹H NMR (400 MHz, DMSO-d₆) δ9.56 (s, 1H), 9.30 (s, 2H), 8.41 (d, J=9.3 Hz, 1H), 7.99 (d, J=5.8 Hz, 1H), 7.39-7.38 (m, 1H), 7.30-7.23 (m, 2H), 6.91-6.89 (m, 1H), 4.62-4.54 (m, 1H), 3.51-3.42 (m, 1H), 2.26-2.21 (m, 1H), 2.14-2.09 (m, 1H), 1.84-1.74 (m, 2H), 1.38-1.32 (m, 10H), 1.35-1.04 (m, 3H); MS (ES+) m/z 470.0 (M+1), 472.0 (M+1).

### Step 2. Preparation of N-((1R,3S)-3-((1-((2-chloropyrimidin-5-yl)amino)isoquinolin-6-yl)oxy)cyclohexyl)-2,2,2-trifluoroacetamide and N-((1R,3S)-3-((1-((2-chloropyrimidin-5-yl)amino)isoquinolin-6-yl)oxy)cyclohexyl)acetamide

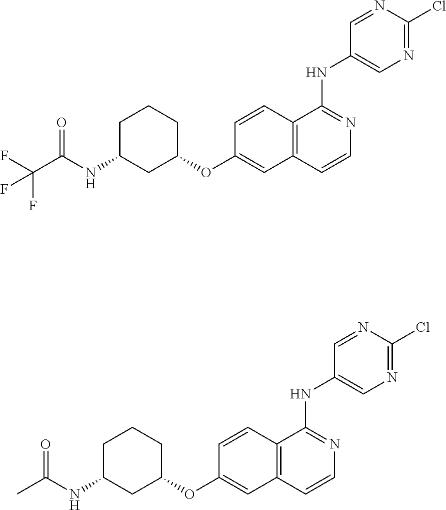 [SMILES: O=C(N[C@@H]1CCC[C@H](Oc2ccc3c(Nc4cnc(Cl)nc4)nccc3c2)C1)C(F)(F)F ; CC(=O)N[C@@H]1CCC[C@H](Oc2ccc3c(Nc4cnc(Cl)nc4)nccc3c2)C1]

To a solution of tert-butyl ((1R,3S)-3-((1-((2-chloropyrimidin-5-yl)amino)isoquinolin-6-yl)oxy)cyclohexyl)carbamate (0.040 g, 0.085 mmol) in dichloromethane (1 mL) was added trifluoroacetic acid (1 mL, 13.1 mmol) at ambient temperature. The mixture was stirred at ambient temperature for 20 minutes and was then concentrated in vacuo to provide a residue. The residue was dissolved in dichloromethane (1 mL), and to it was added triethylamine (0.04 mL, 0.287 mmol) and acetyl chloride (0.06 mL, 0.089 mmol). The mixture was stirred at ambient temperature for 1 h and was then concentrated in vacuo. The obtained residue was purified by silica gel column chromatography, eluting with a gradient of 0 to 100% of ethyl acetate in heptane, to provide the title compounds as colorless solids. As first eluting compound, N-((1R,3S)-3-((1-((2-chloropyrimidin-5-yl)amino)isoquinolin-6-yl)oxy)cyclohexyl)-2,2,2-trifluoroacetamide was obtained (0.018 g, 61% yield): ¹H NMR (400 MHz, DMSO-d₆) δ9.57 (s, 1H), 9.42 (d, J=7.8 Hz, 1H), 9.30 (s, 2H), 8.42 (d, J=9.3 Hz, 1H), 7.99 (d, J=5.8 Hz, 1H), 7.41 (d, J=2.5 Hz, 1H), 7.31 (dd, J=9.2, 2.5 Hz, 1H), 7.23 (d, J=5.8 Hz, 1H), 4.70-4.63 (m, 1H), 3.97-3.87 (m, 1H), 2.30-2.26 (m, 1H), 2.16-2.13 (m, 1H), 1.85-1.81 (m, 2H), 1.56-1.43 (m, 2H), 1.36-1.22 (m, 2H); MS (ES+) m/z 465.9 (M+1), 467.9 (M+1). As second eluting compound, N-((1R,3S)-3-((1-((2-chloropyrimidin-5-yl)amino)isoquinolin-6-yl)oxy)cyclohexyl)acetamide was obtained (0.010 g, 37% yield): ¹H NMR (400 MHz, DMSO-d₆) δ 9.57 (s, 1H), 9.30 (s, 2H), 8.41 (d, J=9.3 Hz, 1H), 7.99 (d, J=5.8 Hz, 1H), 7.86 (d, J=7.6 Hz, 1H), 7.38 (d, J=2.5 Hz, 1H), 7.30 (dd, J=9.2, 2.5 Hz, 1H), 7.23 (d, J=5.8 Hz, 1H), 4.65-4.58 (m, 1H), 3.80-3.72 (m, 1H), 2.28-2.23 (m, 1H), 2.15-2.10 (m, 1H), 1.85-1.75 (m, 5H), 1.50-1.38 (m, 1H), 1.34-1.24 (m, 2H), 1.15-1.04 (m, 1H); MS (ES+) m/z 412.0 (M+1), 414.0 (M+1).

### Examples 377 and 378

### Synthesis of N-((1 S,3R)-3-((1-((2-chloropyrimidin-5-yl)amino)isoquinolin-6-yl)oxy)cyclohexyl)-2,2,2-trifluoroacetamide and N-((1 S,3R)-3-((1-((2-chloropyrimidin-5-yl)amino)isoquinolin-6-yl)oxy)cyclohexyl)acetamide

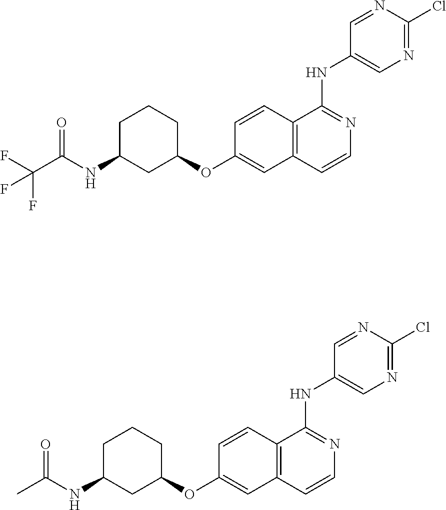 [SMILES: O=C(N[C@H]1CCC[C@@H](Oc2ccc3c(Nc4cnc(Cl)nc4)nccc3c2)C1)C(F)(F)F ; CC(=O)N[C@H]1CCC[C@@H](Oc2ccc3c(Nc4cnc(Cl)nc4)nccc3c2)C1]

Following the procedure as described for EXAMPLES 375 and 376, and making variations as required to replace tert-butyl ((1R,3S)-3-hydroxycyclohexyl)carbamate with tert-butyl ((1 S,3R)-3-hydroxycyclohexyl)carbamate, the title compounds were obtained as colorless solids. As first eluting compound, N-((1S,3R)-3-((1-((2-chloropyrimidin-5-yl)amino)isoquinolin-6-yl)oxy)cyclohexyl)-2,2,2-trifluoroacetamide was obtained (0.004 g, 13% yield): ¹H NMR (400 MHz, CDCl₃) 99.13 (s, 2H), 8.07 (d, J=5.9 Hz, 1H), 7.92 (d, J=9.1 Hz, 1H), 7.19 (dt, J=6.2, 2.9 Hz, 2H), 7.12 (d, J=2.5 Hz, 1H), 4.81-4.77 (m, 1H), 4.29-4.24 (m, 1H), 2.30-2.24 (m, 1H), 1.97-1.85 (m, 3H), 1.76-1.55 (m, 2H), 1.30-1.25 (m, 2H), two NH not observed; MS (ES+) m/z 466.0 (M+1), 468.0 (M+1). As second eluting compound, N-((1S,3R)-3-((1-((2-chloropyrimidin-5-yl)amino)isoquinolin-6-yl)oxy)cyclohexyl)acetamide was obtained (0.011 g, 42% yield): ¹H NMR (400 MHz, DMSO-d₆) δ9.58 (s, 1H), 9.30 (s, 2H), 8.42 (d, J=9.2 Hz, 1H), 7.99 (d, J=5.8 Hz, 1H), 7.86 (d, J=7.7 Hz, 1H), 7.38 (d, J=2.5 Hz, 1H), 7.30 (dd, J=9.2, 2.5 Hz, 1H), 7.23 (d, J=5.9 Hz, 1H), 4.65-4.57 (m, 1H), 3.81-3.71 (m, 1H), 2.27-2.23 (m, 1H), 2.15-2.11 (m, 1H), 1.84-1.76 (m, 5H), 1.49-1.39 (m, 1H), 1.34-1.23 (m, 2H), 1.15-1.05 (m, 1H); MS (ES+) m/z 412.0 (M+1), 414.0 (M+1).

### Example 379

### Synthesis of N-(6-chloropyridin-3-yl)-6-((5-cyclopropyl-1H-pyrazol-4-yl)methoxy)isoquinolin-1-amine

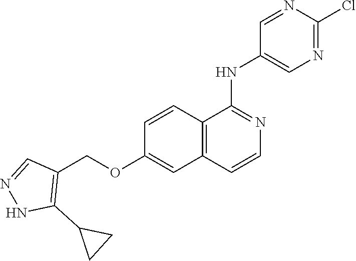 [SMILES: Clc1ncc(Nc2nccc3cc(OCc4cn[nH]c4C4CC4)ccc23)cn1]

### Step 1. Preparation of ethyl 5-cyclopropyl-1-((2-(trimethylsilyl)ethoxy)methyl)-1H-pyrazole-4-carboxylate and ethyl 3-cyclopropyl-1-((2-(trimethylsilyl)ethoxy)methyl)-1H-pyrazole-4-carboxylate

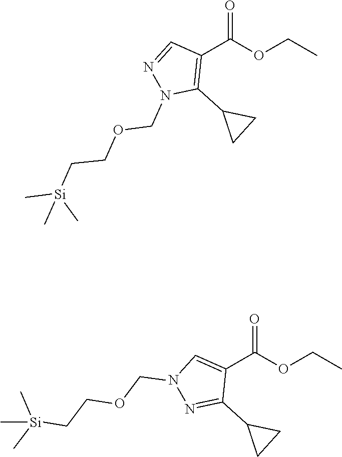

Following the procedure as described for EXAMPLE 334, Step 1 and making variations as required to replace ethyl pyrazole-4-carboxylate with ethyl 3-cyclopropylpyrazole-4-carboxylate, a mixture of the titles compound was obtained as a colorless oil (0.392 g, 73% yield): MS (ES+) m/z 311.6 (M+1), 313.6 (M+1).

### Step 2. Preparation of (5-cyclopropyl-1-((2-(trimethylsilyl)ethoxy)methyl)-1H-pyrazol-4-yl)methanol and (3-cyclopropyl-1-((2-(trimethylsilyl)ethoxy)methyl)-1H-pyrazol-4-yl)methanol

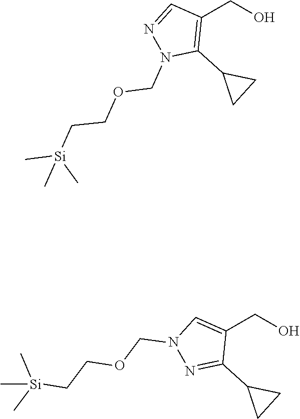 [SMILES: C[Si](C)(C)CCOCn1ncc(CO)c1C1CC1 ; C[Si](C)(C)CCOCn1cc(CO)c(C2CC2)n1]

Following the procedure as described for EXAMPLE 151, Step 1 and making variations as required to replace 1-ethynylcyclopropane-1-carboxylic acid with a mixture of ethyl 5-cyclopropyl-1-((2-(trimethylsilyl)ethoxy)methyl)-1H-pyrazole-4-carboxylate and ethyl 3-cyclopropyl-1-((2-(trimethylsilyl)ethoxy)methyl)-1H-pyrazole-4-carboxylate, a mixture of the title compounds was obtained as a colorless oil (0.341 g, 100% yield): MS (ES+) m/z 269.0 (M+1).

### Step 3. Preparation of N-(6-chloropyridin-3-yl)-6-((5-cyclopropyl-1H-pyrazol-4-yl)methoxy)isoquinolin-1-amine

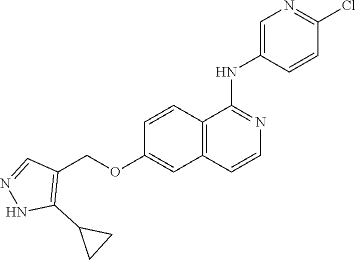 [SMILES: Clc1ccc(Nc2nccc3cc(OCc4cn[nH]c4C4CC4)ccc23)cn1]

Following the procedure as described for EXAMPLE 279, Step 2, and making variations as required to replace (1-(difluoromethyl)-1H-pyrazol-4-yl)methanol with a mixture of (5-cyclopropyl-1-((2-(trimethylsilyl)ethoxy)methyl)-1H-pyrazol-4-yl)methanol and (3-cyclopropyl-1-((2-(trimethylsilyl)ethoxy)methyl)-1H-pyrazol-4-yl)methanol, the title compound was obtained as a colorless solid (0.077 g, 22% yield): ¹H NMR (400 MHz; DMSO-d₆) δ 12.51 (br s, 1H), 9.38 (s, 1H), 8.89 (d, J=2.7 Hz, 1H), 8.44-8.42 (m, 2H), 7.97 (d, J=5.8 Hz, 1H), 7.68 (s, 1H), 7.45 (d, J=8.6 Hz, 2H), 7.30-7.28 (m, 1H), 7.20 (d, J=5.8 Hz, 1H), 5.14 (s, 2H), 1.99-1.94 (m, 1H), 0.90-0.78 (m, 4H); MS (ES+) m/z 392.2 (M+1), 394.2 (M+1).

### Example 380

### Synthesis of N-(6-chloropyridin-3-yl)-6-((1-(2,2-difluoroethyl)-1H-pyrazol-4-yl)methoxy)isoquinolin-1-amine

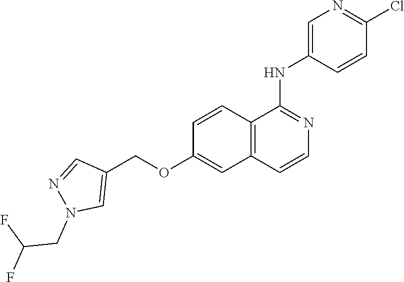 [SMILES: FC(F)Cn1cc(COc2ccc3c(Nc4ccc(Cl)nc4)nccc3c2)cn1]

Following the procedure as described for EXAMPLE 260 and making variations as required to replace 3-(1H-pyrazol-4-yl)propan-1-ol with (1-(2,2-difluoroethyl)-1H-pyrazol-4-yl)methanol, the title compound was obtained as a colorless solid (0.017 g, 15% yield): ¹H NMR (400 MHz, DMSO-d₆) δ 9.38 (s, 1H), 8.88 (d, J=2.8 Hz, 1H), 8.44-8.43 (m, 2H), 7.97 (d, J=4.3 Hz, 2H), 7.69 (s, 1H), 7.45 (d, J=8.7 Hz, 1H), 7.41 (d, J=2.5 Hz, 1H), 7.30-7.27 (m, 1H), 7.20 (d, J=5.9 Hz, 1H), 6.51-6.22 (m, 1H), 5.14 (s, 2H), 4.68-4.60 (m, 2H); ¹⁹F NMR (376 MHz, DMSO-d₆) δ−122.8 (s); MS (ES+) m/z 416.0 (M+1), 418.0 (M+1).

### Example 381

### Synthesis of 6-((1H-pyrazol-4-yl)methoxy)-N-(2-chloropyrimidin-5-yl)isoquinolin-1-amine

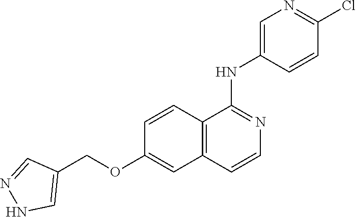 [SMILES: Clc1ccc(Nc2nccc3cc(OCc4cn[nH]c4)ccc23)cn1]

### Step 1. Preparation of 1-chloro-6-((1-((2-(trimethylsilyl)ethoxy)methyl)-1H-pyrazol-4-yl)methoxy)isoquinoline

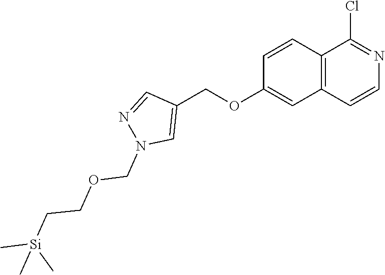 [SMILES: C[Si](C)(C)CCOCn1cc(COc2ccc3c(Cl)nccc3c2)cn1]

Following the procedure as described for EXAMPLE 164, Step 1 and making variations as required to replace (1,5-dimethylpyrazol-4-yl)methanol with (1-((2-(trimethylsilyl)ethoxy)methyl)-1H-pyrazol-4-yl)methanol, the title compound was obtained as a colorless solid (0.347 g, 72% yield): MS (ES+) m/z 390.3 (M+1), 392.3 (M+1).

### Step 2. Preparation of 6-((1H-pyrazol-4-yl)methoxy)-N-(2-chloropyrimidin-5-yl)isoquinolin-1-amine

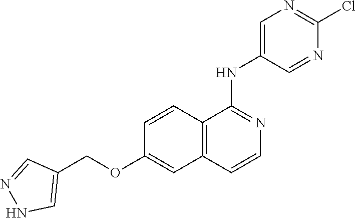 [SMILES: Clc1ncc(Nc2nccc3cc(OCc4cn[nH]c4)ccc23)cn1]

To a solution of 1-chloro-6-((1-((2-(trimethylsilyl)ethoxy)methyl)-1H-pyrazol-4-yl)methoxy)isoquinoline (0.374 g, 0.643 mmol) in 1,4-dioxane (29 mL) was added 5-amino-2-chloropyrimidine (0.0832 g, 0.643 mmol), tris(dibenzylideneacetone)dipalladium(0) (0.0588 g, 0.0643 mmol), 2-dicyclohexylphosphino-2′,6′-dimethoxy-1,1′-biphenyl (0.0527 g, 0.128 mmol), and potassium phosphate tribasic (0.546 g, 2.57 mmol). The mixture was degassed by passing a stream of nitrogen through it for 5 minutes, and then the reaction mixture was heated to 80° C. for 2 h. After cooling to ambient temperature, the reaction mixture was filtered through celite, and the filtrate was concentrated in vacuo. To the obtained residue was added trifluoroacetic acid (0.980 mL, 12.9 mmol) and the reaction mixture was heated to reflux for 4 h. After cooling to ambient temperature, the reaction mixture was concentrated in vacuo. The obtained residue was purified by silica gel column chromatography, eluting with a gradient of 0 to 100% of ethyl acetate in heptane, to provide a residue. To it was added acetonitrile (3 mL), and the mixture was stirred at ambient temperature for 3 minutes and then filtered to afford the title compound as a colorless solid (0.0230 g, 10% yield): ¹H NMR (400 MHz, DMSO-d₆) δ 12.91 (br s, 1H), 9.58-9.58 (m, 1H), 9.30 (s, 2H), 8.43-8.40 (m, 1H), 8.02-7.92 (m, 2H), 7.65 (s, 1H), 7.45 (s, 1H), 7.33-7.30 (m, 1H), 7.28-7.26 (m, 1H), 5.16 (d, J=0.2 Hz, 2H); MS (ES+) m/z 353.0 (M+1), 355.0 (M+1).

### Example 382

### Synthesis of (R)-6-((1,4-dioxan-2-yl)methoxy)-N-(2-chloropyrimidin-5-yl)isoquinolin-1-amine

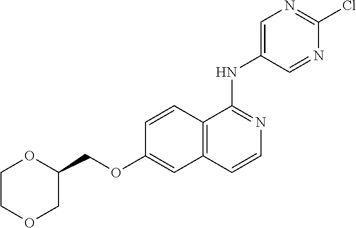 [SMILES: Clc1ncc(Nc2nccc3cc(OC[C@H]4COCCO4)ccc23)cn1]

### Step 1. Preparation of (R)-6-((1,4-dioxan-2-yl)methoxy)-1-chloroisoquinoline

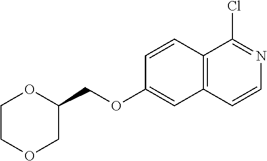 [SMILES: Clc1nccc2cc(OC[C@H]3COCCO3)ccc12]

Following the procedure as described for EXAMPLE 164, Step 1, and making variations as required to replace (1,5-dimethylpyrazol-4-yl)methanol with (S)-(1,4-dioxan-2-yl)methanol, the title compound was obtained as a colorless oil (0.236 g, 51% yield): MS (ES+) m/z 280.2 (M+1), 282.2 (M+1).

### Step 2. Preparation of (R)-6-((1,4-dioxan-2-yl)methoxy)-N-(2-chloropyrimidin-5-yl)isoquinolin-1-amine

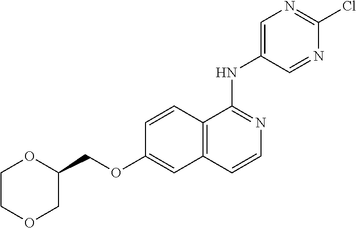 [SMILES: Clc1ncc(Nc2nccc3cc(OC[C@H]4COCCO4)ccc23)cn1]

To a solution (R)-6-((1,4-dioxan-2-yl)methoxy)-1-chloroisoquinoline (0.462 g, 1.65 mmol) in 1,4-dioxane (11 mL) was added 5-amino-2-chloropyrimidine (0.171 g, 1.32 mmol), tris(dibenzylideneacetone)dipalladium(0) (0.151 g, 0.165 mmol), 2-dicyclohexylphosp′in′-2′,6′-dimetho′y-1,1′-biphenyl (0.136 g, 0.330 mmol), and potassium phosphate tribasic (1.05 g, 4.96 mmol). The mixture was degassed by passing a stream of nitrogen through it for 5 minutes, and then the reaction mixture was heated to 100° C. for 1 h. After cooling to ambient temperature, the reaction mixture was filtered through celite, and the filtrate was concentrated in vacuo. The obtained residue was purified by silica gel column chromatography, eluting with a gradient of 0 to 70% of ethyl acetate in heptane. The residue was then purified by reverse-phase column chromatography, eluting with a gradient of 5 to 40% of acetonitrile in water (containing 0.5% of formic acid), to provide the title compound as a colorless solid (0.027 g, 4% yield): ¹H NMR (400 MHz, DMSO-d₆) δ 9.59 (s, 1H), 9.29 (s, 2H), 8.42 (d, J=9.1 Hz, 1H), 8.00 (d, J=5.8 Hz, 1H), 7.35-7.32 (m, 2H), 7.23 (d, J=5.8 Hz, 1H), 4.12 (d, J=4.9 Hz, 2H), 3.96-3.91 (m, 1H), 3.88 (dd, J=11.3, 2.3 Hz, 1H), 3.80-3.77 (m, 1H), 3.67 (ddd, J=10.5, 9.6, 9.4 Hz, 2H), 3.55-3.52 (m, 1H), 3.46 (d, J=11.1 Hz, 1H); MS (ES+) m/z 373.0 (M+1), 375.0 (M+1).

### Example 383

### Synthesis of (S)-6-((1,4-dioxan-2-yl)methoxy)-N-(2-chloropyrimidin-5-yl)isoquinolin-1-amine

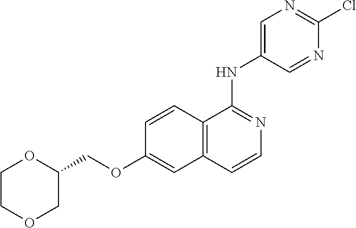 [SMILES: Clc1ncc(Nc2nccc3cc(OC[C@@H]4COCCO4)ccc23)cn1]

### Step 1. Preparation of (S)-6-((1,4-dioxan-2-yl)methoxy)-1-chloroisoquinoline

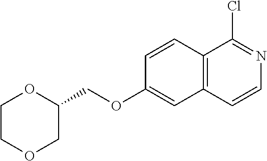 [SMILES: Clc1nccc2cc(OC[C@@H]3COCCO3)ccc12]

Following the procedure as described for EXAMPLE 164, Step 1 and making variations as required to replace (1,5-dimethylpyrazol-4-yl)methanol with (R)-(1,4-dioxan-2-yl)methanol, the title compound was obtained as a colorless oil (0.236 g, 51% yield): MS (ES+) m/z 280.2 (M+1), 282.2 (M+1).

### Step 2. Preparation of (S)-6-((1,4-dioxan-2-yl)methoxy)-N-(2-chloropyrimidin-5-yl)isoquinolin-1-amine

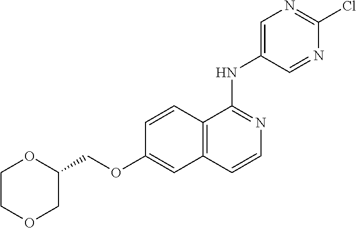 [SMILES: Clc1ncc(Nc2nccc3cc(OC[C@@H]4COCCO4)ccc23)cn1]

Following the procedure as described for EXAMPLE 382, Step 2, and making variations as required to replace (R)-6-((1,4-dioxan-2-yl)methoxy)-1-chloroisoquinoline with (S)-6-((1,4-dioxan-2-yl)methoxy)-1-chloroisoquinoline, the title compound was obtained as a colorless solid (0.351 g, 6% yield): ¹H NMR (400 MHz, DMSO-ds) δ 9.59 (s, 1H), 9.29 (s, 2H), 8.43 (d, J=9.5 Hz, 1H), 8.00 (d, J=5.8 Hz, 1H), 7.35-7.33 (m, 2H), 7.24 (d, J=5.8 Hz, 1H), 4.13 (d, J=4.9 Hz, 2H), 3.96-3.93 (m, 1H), 3.88 (dd, J=11.3, 2.5 Hz, 1H), 3.81-3.78 (m, 1H), 3.67 (qd, J=10.5, 2.2 Hz, 2H), 3.56-3.43 (m, 2H); MS (ES+) m/z 373.0 (M+1), 375.0 (M+1).

### Example 384

### Synthesis of N-(2-chloropyrimidin-5-yl)-6-((1-fluorocyclopropyl)methoxy-d₂)isoquinolin-1-amine

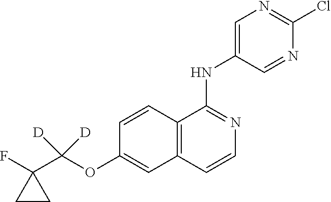 [SMILES: [2H]C([2H])(Oc1ccc2c(Nc3cnc(Cl)nc3)nccc2c1)C1(F)CC1]

### Step 1. Preparation of 1-chloro-6-((1-fluorocyclopropyl)methoxy-d₂)isoquinoline

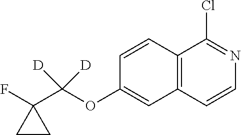 [SMILES: [2H]C([2H])(Oc1ccc2c(Cl)nccc2c1)C1(F)CC1]

To a solution of 1-fluorocyclopropane-1-carboxylic acid (1.00 g, 9.61 mmol) in tetrahydrofuran (32 mL) was slowly added borane-d₃ (1 M in tetrahydrofuran, 19 mL, 19.2 mmol) at 0° C. The reaction mixture was allowed to warm to ambient temperature and stirred for 16 h.

To the mixture was then added methanol (15 mL), and the resulting mixture was concentrated in vacuo, and this process was repeated twice. The obtained residue was dissolved in N,N-dimethylformamide (32 mL), and to the resulting mixture was added 1-chloro-6-fluoroisoquinoline (1.39 g, 7.68 mmol) and potassium tert-butoxide (1 M in tetrahydrofuran, 9.60 mL, 9.60 mmol) at ambient temperature. The reaction mixture was stirred at ambient temperature for 16 h and then concentrated in vacuo. The obtained residue was purified by silica gel column chromatography, eluting with a gradient of 0 to 30% of ethyl acetate in heptane, to provide the title compound as a colourless solid (0.430, 18% yield): ¹H NMR (400 MHz, CDCl₃) 98.25 (d, J=9.3 Hz, 1H), 8.19 (d, J=5.7 Hz, 1H), 7.47 (d, J=5.7 Hz, 1H), 7.37 (dd, J=9.3, 2.5 Hz, 1H), 7.09 (d, J=2.5 Hz, 1H), 1.27 (dt, J=18.5, J=7.3 Hz, 2H), 0.89 (q, J=7.8 Hz, 2H); ¹⁹F NMR (376 MHz, CDCl₃) δ−188.2 (s); MS (ES+) m/z 254.4 (M+1), 256.4 (M+1).

### Step 2. Preparation of N-(2-chloropyrimidin-5-yl)-6-((1-fluorocyclopropyl)methoxy-d₂)isoquinolin-1-amine

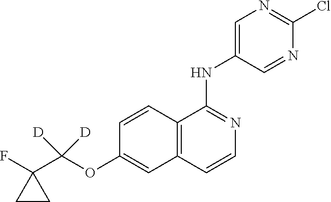 [SMILES: [2H]C([2H])(Oc1ccc2c(Nc3cnc(Cl)nc3)nccc2c1)C1(F)CC1]

Following the procedure as described for EXAMPLE 382, Step 2, and making variations as required to replace (R)-6-((1,4-dioxan-2-yl)methoxy)-1-chloroisoquinoline with 1-chloro-6-((1-fluorocyclopropyl)methoxy-d₂)isoquinoline, the title compound was obtained as a colorless oil (0.173 g, 29% yield): ¹H NMR (400 MHz, DMSO-d₆) δ9.58 (s, 1H), 9.30 (s, 2H), 8.44 (d, J=9.3 Hz, 1H), 8.00 (d, J=5.8 Hz, 1H), 7.41 (d, J=2.5 Hz, 1H), 7.34 (d, J=2.4 Hz, 1H), 7.22 (d, J=5.8 Hz, 1H), 1.18 (dt, J=18.6, 6.9 Hz, 2H), 0.92 (q, J=7.4 Hz, 2H); ¹⁹F NMR (376 MHz, DMSO-ds) δ−185.8 (s); MS (ES+) m/z 347.0 (M+1), 349.0 (M+1).

### Example 385

### Synthesis of 1-(1-((1-((2-chloropyrimidin-5-yl)amino)isoquinolin-6-yl)oxy)ethyl)cyclopropane-1-carbonitrile

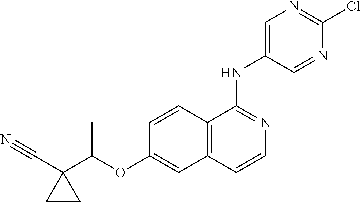 [SMILES: CC(Oc1ccc2c(Nc3cnc(Cl)nc3)nccc2c1)C1(C#N)CC1]

### Step 1. Preparation of 1-(1-hydroxyethyl)cyclopropane-1-carbonitrile

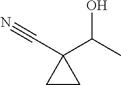

Following the procedure as described for EXAMPLE 335, Step 2 and making variations as required to replace 1-(1-((2-(trimethylsilyl)ethoxy)methyl)-1H-pyrazol-4-yl)ethan-1-one with 1-acetylcyclopropane-1-carbonitrile, the title compound was obtained as a yellow oil (0.242 g, 95% yield): ¹H NMR (400 MHz, CDCl₃) δ 3.34 (q, J=5.9 Hz, 1H), 1.47 (d, J=6.3 Hz, 3H), 1.30-1.23 (m, 2H), 1.08-1.04 (m, 1H), 0.94-0.90 (m, 1H), OH not observed.

### Step 2. Preparation of 1-(1-((1-chloroisoquinolin-6-yl)oxy)ethyl)cyclopropane-1-carbonitrile

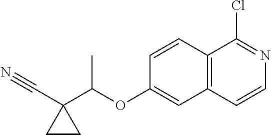 [SMILES: CC(Oc1ccc2c(Cl)nccc2c1)C1(C#N)CC1]

To a solution of 1-(1-hydroxyethyl)cyclopropane-1-carbonitrile (0.240 mg, 2.16 mmol) in N,N-dimethylformamide (8.6 mL) was added 1-chloro-6-fluoroisoquinoline (0.412 g, 2.27 mmol) and potassium tert-butoxide (1 M in tetrahydrofuran, 2.38 mL, 2.38 mmol), and the resulting mixture was stirred at ambient temperature for 16 h. The mixture was concentrated in vacuo.

The obtained residue was purified by silica gel column chromatography, eluting with a gradient of 0 to 50% of ethyl acetate in heptane, to afford the title compound as a colorless solid (0.263 g, 45% yield): ¹H NMR (400 MHz, CDCl₃) δ 8.26 (d, J=9.2 Hz, 1H), 8.20 (d, J=5.7 Hz, 1H), 7.46 (d, J=5.7 Hz, 1H), 7.30 (dd, J=9.2, 2.5 Hz, 1H), 7.07 (d, J=2.5 Hz, 1H), 4.18 (q, J=6.2 Hz, 1H), 1.64 (d, J=6.2 Hz, 3H), 1.37-1.35 (m, 2H), 1.15-1.11 (m, 1H), 1.07-1.03 (m, 1H); MS (ES+) m/z 273.0 (M+1), 275.0 (M+1).

### Step 3. Preparation of 1-(1-((1-((2-chloropyrimidin-5-yl)amino)isoquinolin-6-yl)oxy)ethyl)cyclopropane-1-carbonitrile

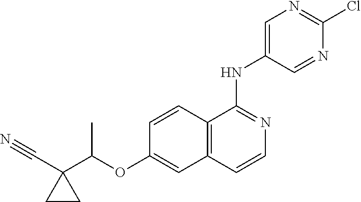 [SMILES: CC(Oc1ccc2c(Nc3cnc(Cl)nc3)nccc2c1)C1(C#N)CC1]

Following the procedure as described for EXAMPLE 382, Step 2, and making variations as required to replace (R)-6-((1,4-dioxan-2-yl)methoxy)-1-chloroisoquinoline with 1-(1-((1-chloroisoquinolin-6-yl)oxy)ethyl)cyclopropane-1-carbonitrile, the title compound was obtained as a colorless oil (0.082 g, 23% yield): ¹H NMR (400 MHz, DMSO-d₆) δ9.60 (s, 1H), 9.29 (s, 2H), 8.45 (d, J=9.1 Hz, 1H), 8.00 (d, J=5.8 Hz, 1H), 7.37-7.34 (m, 2H), 7.20 (d, J=5.9 Hz, 1H), 4.34 (q, J=6.1 Hz, 1H), 1.49 (d, J=6.1 Hz, 3H), 1.35-1.32 (m, 2H), 1.19-1.15 (m, 2H); MS (ES+) m/z 366.0 (M+1), 368.0 (M+1).

### Example 386

### Synthesis of N-(6-chloropyridin-3-yl)-6-((1-methyl-1H-pyrazol-4-yl)methoxy-d₂)isoquinolin-1-amine

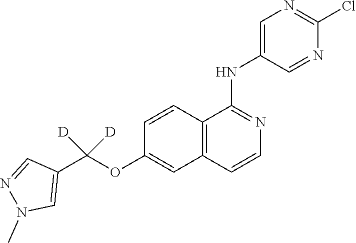 [SMILES: [2H]C([2H])(Oc1ccc2c(Nc3cnc(Cl)nc3)nccc2c1)c1cnn(C)c1]

### Step 1. Preparation of (1-methyl-1H-pyrazol-4-yl)methan-d₂-ol

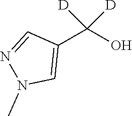

To a solution of 1-methyl-1H-pyrazole-4-carboxylic acid (1.00 g, 7.93 mmol) in tetrahydrofuran (32 mL) was added borane-d₃ (1 M in tetrahydrofuran, 15.9 mL, 15.9 mmol) at 0° C. The resulting mixture was allowed to warm to ambient temperature and stirred for 5 h. To the reaction mixture was then added methanol (15 mL). The resulting mixture was stirred for 30 minutes and then concentrated in vacuo. The obtained residue was diluted with saturated sodium carbonate solution (30 mL) and extracted with ethyl acetate (3×30 mL). The combined organic phase was dried over anhydrous sodium sulfate and filtered, and the filtrate was concentrated in vacuo to afford the title compound as a colorless oil (0.194 g, 21% yield): ¹H NMR (400 MHz, CDCl₃) δ7.49 (s, 1H), 7.40 (s, 1H), 3.90 (s, 3H), OH not observed.

### Step 2. Preparation of N-(6-chloropyridin-3-yl)-6-((1-methyl-1H-pyrazol-4-yl)methoxy-d₂)isoquinolin-1-amine

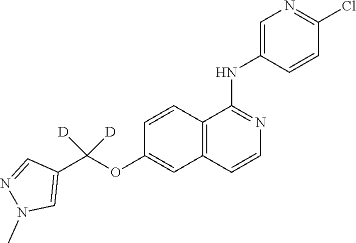 [SMILES: [2H]C([2H])(Oc1ccc2c(Nc3ccc(Cl)nc3)nccc2c1)c1cnn(C)c1]

Following the procedure as described for EXAMPLE 279, Step 2, and making variations as required to replace (1-(difluoromethyl)-1H-pyrazol-4-yl)methanol with (1-methyl-1H-pyrazol-4-yl)methan-d₂-ol, the title compound was obtained as a colorless solid (0.306 g, 53% yield): ¹H NMR (400 MHz, DMSO-d₆) δ 9.38 (s, 1H), 8.88 (d, J=2.8 Hz, 1H), 8.43 (d, J=8.9 Hz, 2H), 7.97 (d, J=5.8 Hz, 1H), 7.87 (s, 1H), 7.58 (s, 1H), 7.45 (d, J=8.7 Hz, 1H), 7.40 (d, J=2.6 Hz, 1H), 7.27 (dd, J=9.2, 2.6 Hz, 1H), 7.20 (d, J=5.8 Hz, 1H), 3.84 (s, 3H); MS (ES+) m/z 368.0 (M+1), 370.0 (M+1).

### Example 387

### Synthesis of N-(6-chloropyridin-3-yl)-6-(2-(pyrimidin-2-yl)ethoxy)isoquinolin-1-amine

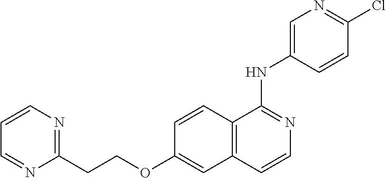 [SMILES: Clc1ccc(Nc2nccc3cc(OCCc4ncccn4)ccc23)cn1]

Following the procedure as described for EXAMPLE 260 and making variations as required to replace 3-(1H-pyrazol-4-yl)propan-1-ol with 2-(pyrimidin-2-yl)ethan-1-ol, the title compound was obtained as a colorless solid (0.007 g, 5% yield): ¹H NMR (400 MHz, DMSO-d₆) δ9.38 (s, 1H), 8.88 (d, J=2.8 Hz, 1H), 8.79 (d, J=4.9 Hz, 2H), 8.42 (td, J=6.1, 2.9 Hz, 2H), 7.97-7.96 (m, 1H), 7.46-7.44 (m, 1H), 7.41 (dd, J=6.3, 3.5 Hz, 1H), 7.35 (d, J=2.5 Hz, 1H), 7.22 (dd, J=6.4, 4.3 Hz, 2H), 4.65 (t, J=6.4 Hz, 2H), 3.45 (t, J=6.3 Hz, 2H); MS (ES+) m/z 378.2 (M+1), 380.2 (M+1).

### Example 388

### Synthesis of N-(2-methoxypyrimidin-5-yl)-6-((5-methyl-1H-pyrazol-4-yl)methoxy)isoquinolin-1-amine

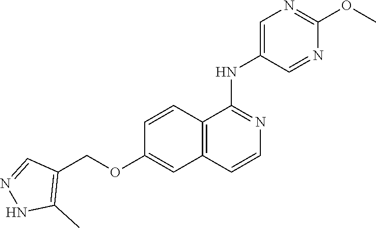 [SMILES: COc1ncc(Nc2nccc3cc(OCc4cn[nH]c4C)ccc23)cn1]

To a solution of 6-fluoro-N-(2-methoxypyrimidin-5-yl)-N-((2-(trimethylsilyl)ethoxy)methyl)isoquinolin-1-amine (0.075 g, 0.165 mmol) in N,N-dimethylformamide (1.5 mL) was added a mixture of (3-methyl-1-((2-(trimethylsilyl)ethoxy)methyl)-1H-pyrazol-4-yl)methanol and (5-methyl-1-((2-(trimethylsilyl)ethoxy)methyl)-1H-pyrazol-4-yl)methanol (0.048 g, 0.198 mmol) and potassium tert-butoxide (1 M in tetrahydrofuran, 0.247 mL, 0.247 mmol) at ambient temperature, and the resulting mixture was heated to 80° C. for 1 h. After cooling to ambient temperature, the mixture was concentrated in vacuo and the obtained residue was dissolved in dichloromethane (3 mL). To this mixture was added trifluoroacetic acid (0.252 mL, 3.30 mmol), and the reaction mixture was heated to reflux for 5 h. After cooling to ambient temperature, the reaction mixture was concentrated in vacuo to provide a residue which was purified by silica gel column chromatography, eluting with a gradient of 0 to 100% of ethyl acetate in heptane, to afford the title compound as a colorless solid (0.003 g, 5% yield): ¹H NMR (400 MHz, DMSO-d₆) δ8.93 (s, 2H), 8.45 (d, J=9.1 Hz, 1H), 7.78 (d, J=5.9 Hz, 1H), 7.69 (s, 1H), 7.48 (s, 1H), 7.35 (d, J=8.5 Hz, 1H), 7.20 (d, J=6.1 Hz, 1H), 5.11 (s, 2H), 3.94 (s, 3H), 2.25 (s, 3H), two NH not observed; MS (ES+) m/z 363.0 (M+1).

### Example 389

### Synthesis of 6-(3-(1H-pyrazol-4-yl)propoxy)-N-(2-chloropyrimidin-5-yl)isoquinolin-1-amine

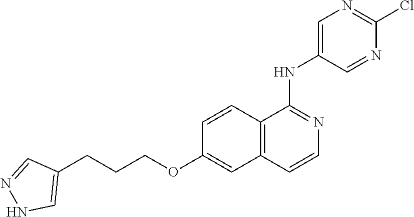 [SMILES: Clc1ncc(Nc2nccc3cc(OCCCc4cn[nH]c4)ccc23)cn1]

### Step 1. Preparation of 6-(3-(1H-pyrazol-4-yl)propoxy)-1-chloroisoquinoline

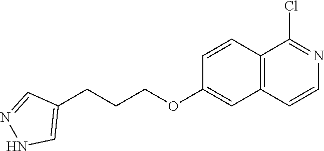 [SMILES: Clc1nccc2cc(OCCCc3cn[nH]c3)ccc12]

Following the procedure as described for EXAMPLE 156, Step 1, and making variations as required to replace (5-methylisoxazol-4-yl)methanol with 3-(1H-pyrazol-4-yl)propan-1-ol, the title compound was obtained as a colorless solid (0.324 g, 100% yield): MS (ES+) m/z 288.4 (M+1), 290.4 (M+1).

### Step 2. Preparation of 6-(3-(1H-pyrazol-4-yl)propoxy)-N-(2-chloropyrimidin-5-yl)isoquinolin-1-amine

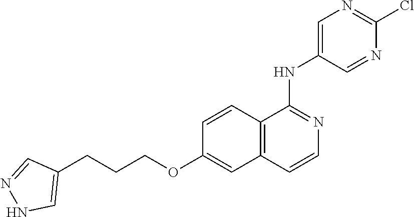 [SMILES: Clc1ncc(Nc2nccc3cc(OCCCc4cn[nH]c4)ccc23)cn1]

Following the procedure as described for EXAMPLE 382, Step 2, and making variations as required to replace (R)-6-((1,4-dioxan-2-yl)methoxy)-1-chloroisoquinoline with 6-(3-(1H-pyrazol-4-yl)propoxy)-1-chloroisoquinoline, the title compound was obtained as a yellow solid (0.032 g, 7% yield): ¹H NMR (400 MHz, DMSO-d₆) δ 9.27 (s, 2H), 8.43 (d, J=9.1 Hz, 1H), 7.97 (d, J=6.0 Hz, 1H), 7.65-7.53 (m, 2H), 7.47 (s, 2H), 7.35-7.31 (m, 2H), 7.24 (d, J=5.8 Hz, 1H), 4.14 (t, J=6.4 Hz, 2H), 2.64 (t, J=7.6 Hz, 2H), 2.05 (quintet, J=7.1 Hz, 2H); MS (ES+) m/z 381.0 (M+1), 383.0 (M+1).

### Example 390

### Synthesis of 6-(2-(1H-pyrazol-4-yl)ethoxy)-N-(2-chloropyrimidin-5-yl)isoquinolin-1-amine formate

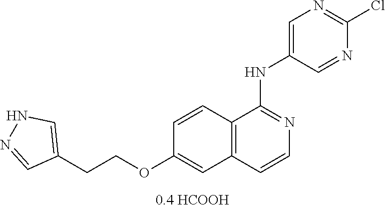 [SMILES: Clc1ncc(Nc2nccc3cc(OCCc4cn[nH]c4)ccc23)cn1]

### Step 1. Preparation of 6-(2-(1H-pyrazol-4-yl)ethoxy)-1-chloroisoquinoline

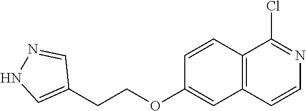 [SMILES: Clc1nccc2cc(OCCc3cn[nH]c3)ccc12]

Following the procedure as described for EXAMPLE 156, Step 1, and making variations as required to replace (5-methylisoxazol-4-yl)methanol with 2-(1H-pyrazol-4-yl)-ethanol, the title compound was obtained as a colorless solid (0.278 g, 83% yield): MS (ES+) m/z 274.0 (M+1), 276.0 (M+1).

### Step 2. Preparation of 6-(2-(1H-pyrazol-4-yl)ethoxy)-N-(2-chloropyrimidin-5-yl)isoquinolin-1-amine formate

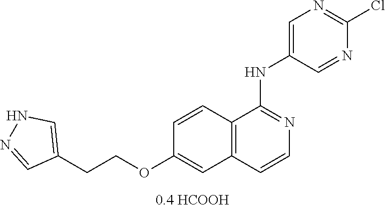 [SMILES: Clc1ncc(Nc2nccc3cc(OCCc4cn[nH]c4)ccc23)cn1]

Following the procedure as described for EXAMPLE 382, Step 2, and making variations as required to replace (R)-6-((1,4-dioxan-2-yl)methoxy)-1-chloroisoquinoline with 6-(2-(1H-pyrazol-4-yl)ethoxy)-1-chloroisoquinoline, the title compound was obtained as a colorless solid (0.034 g, 10% yield): ¹H NMR (400 MHz, DMSO-d₆) δ 12.67 (s, 1H), 9.58 (s, 1H), 9.29 (s, 2H), 8.42 (d, J=10.0 Hz, 1H), 8.20 (s, 0.4H), 7.99 (d, J=6.1 Hz, 1H), 7.57 (s, 2H), 7.34 (d, J=7.6 Hz, 2H), 7.24 (d, J=6.0 Hz, 1H), 4.26 (t, J=6.6 Hz, 2H), 2.97 (t, J=6.5 Hz, 2H), COOH not observed; MS (ES+) m/z 367.0 (M+1), 369.0 (M+1).

### Example 391

### Synthesis of 6-((1H-pyrazol-3-yl)methoxy)-N-(6-chloropyridin-3-yl)isoquinolin-1-amine

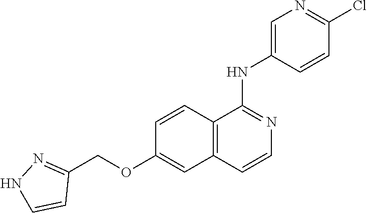 [SMILES: Clc1ccc(Nc2nccc3cc(OCc4cc[nH]n4)ccc23)cn1]

### Step 1. Preparation of 6-((1H-pyrazol-3-yl)methoxy)-1-chloroisoquinoline

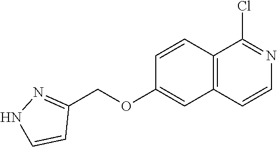 [SMILES: Clc1nccc2cc(OCc3cc[nH]n3)ccc12]

To a mixture of 1-chloroisoquinolin-6-ol (0.300 g, 1.67 mmol) and 3-(chloromethyl)-1H-pyrazole hydrochloride (0.307 g, 2.00 mmol) in N,N-dimethylformamide (10 mL) was added potassium carbonate (1.15 g, 8.35 mmol), and the mixture was heated to 90° C. for 2 h. After cooling to ambient temperature, the reaction mixture was concentrated in vacuo. The obtained residue was poured into water (20 mL) and the mixture was extracted with ethyl acetate (3×15 mL). The combined organic phase was dried over anhydrous sodium sulfate, filtered, and the filtrate was concentrated in vacuo. The obtained residue was purified by silica gel column chromatography, eluting with 33% of ethyl acetate in petroleum ether, to give the title compound as a dark solid (0.220 g, 42% yield): ¹H NMR (400 MHz, DMSO-d₆) δ 13.02-12.78 (m, 1H), 8.23-8.14 (m, 2H), 7.76 (d, J=5.6 Hz, 1H), 7.70 (s, 1H), 7.64 (d, J=2.4 Hz, 1H), 7.45 (dd, J=2.4, 9.2 Hz, 1H), 6.43 (d, J=2.0 Hz, 1H), 5.26 (s, 2H); MS (ES+) m/z 260.2 (M+1), 262.2 (M+1).

### Step 2. Preparation of 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline

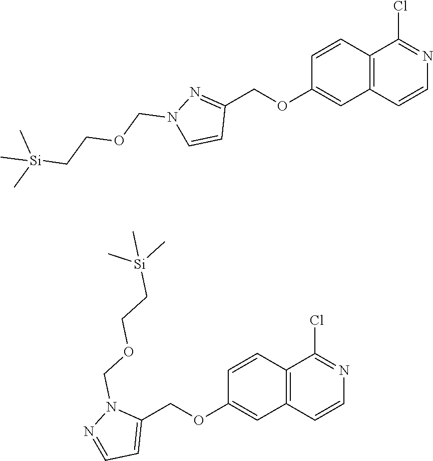 [SMILES: C[Si](C)(C)CCOCn1ccc(COc2ccc3c(Cl)nccc3c2)n1 ; C[Si](C)(C)CCOCn1nccc1COc1ccc2c(Cl)nccc2c1]

Following the procedure as described for EXAMPLE 334, Step 1, and making variations as required to replace ethyl pyrazole-4-carboxylate with 6-((1H-pyrazol-3-yl)methoxy)-1-chloroisoquinoline, a mixture of the title compounds was obtained as a yellowish solid (0.220 g, 57% yield): MS (ES+) m/z 390.2 (M+1), 392.2 (M+1).

### Step 3. Preparation of N-(6-chloropyridin-3-yl)-6-((1-((2-(trimethylsilyl)ethoxy)methyl)-1H-pyrazol-3-yl)methoxy)isoquinolin-1-amine and N-(6-chloropyridin-3-yl)-6-((1-((2-(trimethylsilyl)ethoxy)methyl)-1H-pyrazol-5-yl)methoxy)isoquinolin-1-amine

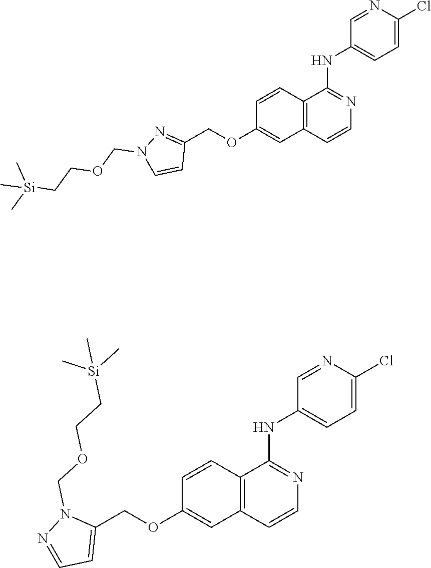 [SMILES: C[Si](C)(C)CCOCn1ccc(COc2ccc3c(Nc4ccc(Cl)nc4)nccc3c2)n1 ; C[Si](C)(C)CCOCn1nccc1COc1ccc2c(Nc3ccc(Cl)nc3)nccc2c1]

To a mixture of 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline (0.220 mg, 0.564 mmol), 6-chloropyridin-3-amine (0.0870 g, 0.677 mmol), and cesium carbonate (0.551 g, 1.69 mmol) in 2-methyl-2-butanol (6 mL) was added [(2-di-tert-butylphosphino-2′,4′,6′-triisopropyl-1,1′-biphenyl)-2-(2′-amino-1,1′-biphenyl)]palladium(II) methanesulfonate (0.045 g, 0.056 mmol) at ambient temperature. The mixture was heated to 90° C. for 12 h. After cooling to ambient temperature, the reaction mixture was concentrated in vacuo. The obtained residue was poured into water (20 mL) and extracted with ethyl acetate (3×15 mL). The combined organic phase was dried over anhydrous sodium sulfate, filtered, and the filtrate was concentrated in vacuo. The residue was purified by silica gel column chromatography, eluting with 17% of ethyl acetate in petroleum ether, to give a mixture of the title compounds as a yellowish solid (0.120 g, 44% yield).

### Step 4. Preparation of 6-((1H-pyrazol-3-yl)methoxy)-N-(6-chloropyridin-3-yl)isoquinolin-1-amine

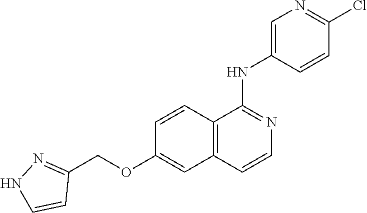 [SMILES: Clc1ccc(Nc2nccc3cc(OCc4cc[nH]n4)ccc23)cn1]

To a mixture of a mixture of N-(6-chloropyridin-3-yl)-6-((1-((2-(trimethylsilyl)ethoxy)methyl)-1H-pyrazol-3-yl)methoxy)isoquinolin-1-amine and N-(6-chloropyridin-3-yl)-6-((1-((2-(trimethylsilyl)ethoxy)methyl)-1H-pyrazol-5-yl)methoxy)isoquinolin-1-amine in dichloromethane (2 mL) was added trifluoroacetic acid (1.54 g, 13.5 mmol), and the mixture was stirred at ambient temperature for 2 h. The residue was poured into water (20 mL) and extracted with ethyl acetate (3×15 mL). The combined organic phase was dried over anhydrous sodium sulfate, filtered, and the filtrate was concentrated in vacuo. The residue was purified by reverse-phase preparative HPLC (Waters XBridge 150 mm×25 mm, 5 μm column), eluting with a gradient of 33 to 63% of acetonitrile in water (containing 10 mM of ammonium formate), to afford the title compound as a colorless solid (0.009 g, 12% yield): ¹H NMR (400 MHz, DMSO-d₆) δ13.07-12.73 (m, 1H), 9.37 (s, 1H), 8.87 (d, J=2.8 Hz, 1H), 8.47-8.38 (m, 2H), 7.97 (d, J=5.6 Hz, 1H), 7.78-7.65 (m, 1H), 7.48-7.40 (m, 2H), 7.31 (dd, J=2.4, 9.2 Hz, 1H), 7.18 (d, J=5.6 Hz, 1H), 6.41 (d, J=2.4 Hz, 1H), 5.22 (s, 2H); MS (ES+) m/z 352.2 (M+1), 354.2 (M+1).

### Example 392

### Synthesis of N-(6-chloropyridin-3-yl)-6-((5-methyl-1H-pyrazol-3-yl)methoxy)isoquinolin-1-amine

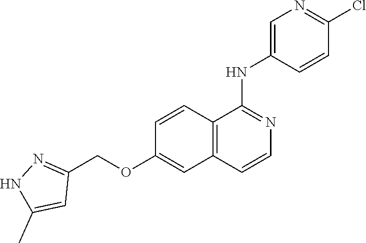 [SMILES: Cc1cc(COc2ccc3c(Nc4ccc(Cl)nc4)nccc3c2)n[nH]1]

### Step 1. Preparation of 3-(chloromethyl)-5-methyl-1H-pyrazole

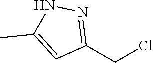

A mixture of (5-methyl-1H-pyrazol-3-yl)methanol (0.450 g, 4.01 mmol) in thionyl chloride (4 mL) was degassed by passing a stream of nitrogen gas for 5 minutes. The mixture was heated to 80° C. for 1 h. The reaction mixture was concentrated in vacuo to provide the title compound as a yellow solid (0.600 g, quantitative yield): ¹H NMR (400 MHz, CD₃OD) δ6.65 (s, 1H), 4.81 (s, 2H), 2.47 (s, 3H), NH not observed.

### Step 2. Preparation of 1-chloro-6-((5-methyl-1H-pyrazol-3-yl)methoxy)isoquinoline

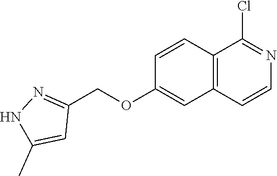 [SMILES: Cc1cc(COc2ccc3c(Cl)nccc3c2)n[nH]1]

Following the procedure as described for EXAMPLE 391, Step 1, and making variations as required to replace 3-(chloromethyl)-1H-pyrazole hydrochloride with 3-(chloromethyl)-5-methyl-1H-pyrazole, the title compound was obtained as a yellowish oil (0.310 g, 47% yield): ¹H NMR (400 MHz, CD₃OD) δ 8.25 (d, J=9.2 Hz, 1H), 8.12 (d, J=5.6 Hz, 1H), 7.69 (d, J=5.6 Hz, 1H), 7.47 (d, J=2.4 Hz, 1H), 7.44-7.39 (m, 1H), 6.20 (s, 1H), 5.21 (s, 2H), 2.30 (s, 3H), NH not observed.

### Step 3. Preparation of 1-chloro-6-((5-methyl-1-((2-(trimethylsilyl)ethoxy)methyl)-1H-pyrazol-3-yl)methoxy)isoquinoline and 1-chloro-6-((3-methyl-1-((2-(trimethylsilyl)ethoxy)methyl)-1H-pyrazol-5-yl)methoxy)isoquinoline

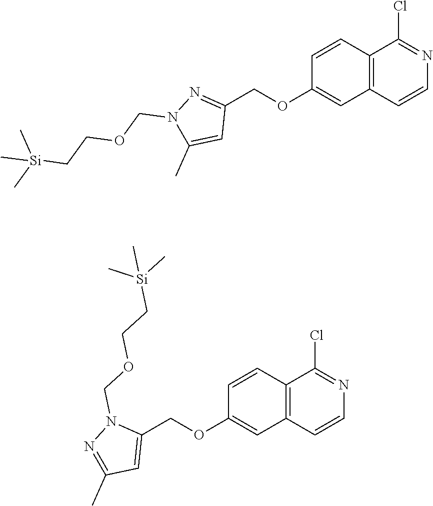 [SMILES: Cc1cc(COc2ccc3c(Cl)nccc3c2)nn1COCC[Si](C)(C)C ; Cc1cc(COc2ccc3c(Cl)nccc3c2)n(COCC[Si](C)(C)C)n1]

Following the procedure as described for EXAMPLE 391, Step 2, and making variations as required to replace 6-((1H-pyrazol-3-yl)methoxy)-1-chloroisoquinoline with 1-chloro-6-((5-methyl-1H-pyrazol-3-yl)methoxy)isoquinoline, a mixture of the title compounds was obtained as a yellowish oil (0.300 g, 75% yield).

### Step 4. Preparation of N-(6-chloropyridin-3-yl)-6-((5-methyl-1-((2-(trimethylsilyl)ethoxy)methyl)-1H-pyrazol-3-yl)methoxy)isoquinolin-1-amine and N-(6-chloropyridin-3-yl)-6-((3-methyl-1-((2-(trimethylsilyl)ethoxy)methyl)-1H-pyrazol-5-yl)methoxy)isoquinolin-1-amine

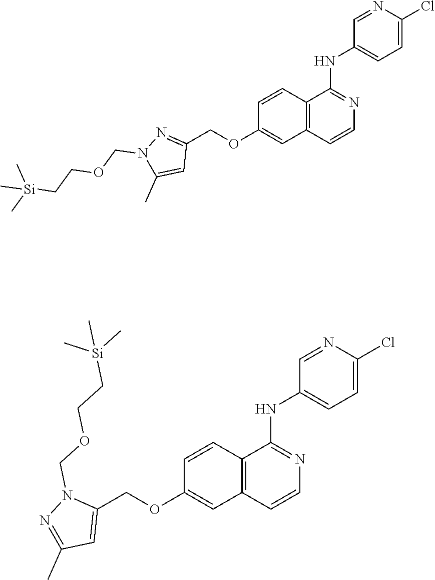 [SMILES: Cc1cc(COc2ccc3c(Nc4ccc(Cl)nc4)nccc3c2)nn1COCC[Si](C)(C)C ; Cc1cc(COc2ccc3c(Nc4ccc(Cl)nc4)nccc3c2)n(COCC[Si](C)(C)C)n1]

Following the procedure as described for EXAMPLE 391, Step 3, and making variations as required to replace 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline with 1-chloro-6-((5-methyl-1-((2-(trimethylsilyl)ethoxy)methyl)-1H-pyrazol-3-yl)methoxy)isoquinoline and 1-chloro-6-((3-methyl-1-((2-(trimethylsilyl)ethoxy)methyl)-1H-pyrazol-5-yl)methoxy)isoquinoline, a mixture of the title compounds was obtained as a yellowish oil (0.200 g, 74% yield).

### Step 5. Preparation of N-(6-chloropyridin-3-yl)-6-((5-methyl-1H-pyrazol-3-yl)methoxy)isoquinolin-1-amine

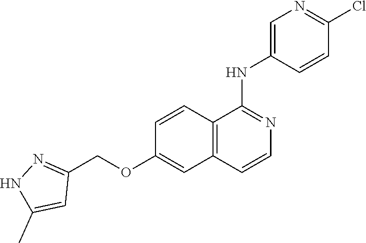 [SMILES: Cc1cc(COc2ccc3c(Nc4ccc(Cl)nc4)nccc3c2)n[nH]1]

Following the procedure as described for EXAMPLE 391, Step 4 and making variations as required to replace the mixture of N-(6-chloropyridin-3-yl)-6-((1-((2-(trimethylsilyl)ethoxy)methyl)-1H-pyrazol-3-yl)methoxy)isoquinolin-1-amine and N-(6-chloropyridin-3-yl)-6-((1-((2-(trimethylsilyl)ethoxy)methyl)-1H-pyrazol-5-yl)methoxy)isoquinolin-1-amine with a mixture of N-(6-chloropyridin-3-yl)-6-((5-methyl-1-((2-(trimethylsilyl)ethoxy)methyl)-1H-pyrazol-3-yl)methoxy)isoquinolin-1-amine and N-(6-chloropyridin-3-yl)-6-((3-methyl-1-((2-(trimethylsilyl)ethoxy)methyl)-1H-pyrazol-5-yl)methoxy)isoquinolin-1-amine, the title compound was obtained as a colorless solid (0.027 g, 20% yield): ¹H NMR (400 MHz, DMSO-d₆) δ 11.64 (s, 1H), 8.89 (d, J=9.6 Hz, 1H), 8.65 (d, J=2.8 Hz, 1H), 8.08 (dd, J=2.8, 8.8 Hz, 1H), 7.73 (d, J=8.4 Hz, 1H), 7.66 (d, J=2.4 Hz, 1H), 7.59 (d, J=6.8 Hz, 1H), 7.51 (dd, J=2.4, 9.2 Hz, 1H), 7.34 (d, J=6.8 Hz, 1H), 6.22 (s, 1H), 5.26 (s, 2H), 2.25 (s, 3H), NH not observed; MS (ES+) m/z 366.2 (M+1), 368.2 (M+1).

### Example 393

### Synthesis of N-(6-chloropyridin-3-yl)-6-((3-methyl-1H-pyrazol-4-yl)methoxy)isoquinolin-1-amine

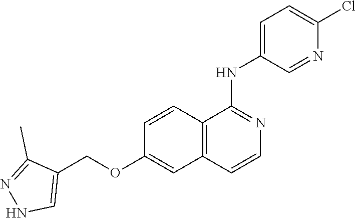 [SMILES: Cc1n[nH]cc1COc1ccc2c(Nc3ccc(Cl)nc3)nccc2c1]

### Step 1. Preparation of N-(6-chloropyridin-3-yl)-6-((3-methyl-1-((2-(trimethylsilyl)ethoxy)methyl)-1H-pyrazol-4-yl)methoxy)isoquinolin-1-amine and N-(6-chloropyridin-3-yl)-6-((5-methyl-1-((2-(trimethylsilyl)ethoxy)methyl)-1H-pyrazol-4-yl)methoxy)isoquinolin-1-amine

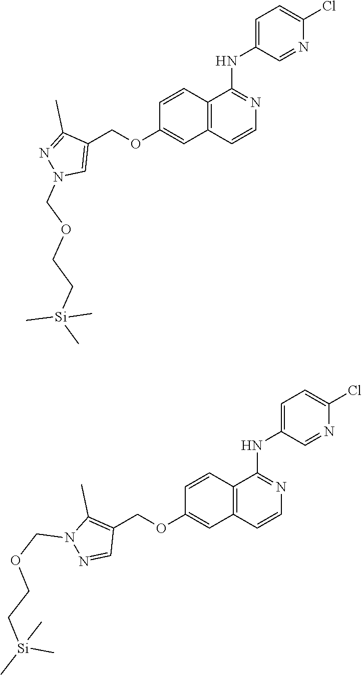 [SMILES: Cc1nn(COCC[Si](C)(C)C)cc1COc1ccc2c(Nc3ccc(Cl)nc3)nccc2c1 ; Cc1c(COc2ccc3c(Nc4ccc(Cl)nc4)nccc3c2)cnn1COCC[Si](C)(C)C]

A mixture of 1-chloro-6-((3-methyl-1-((2-(trimethylsilyl)ethoxy)methyl)-1H-pyrazol-4-yl)methoxy)isoquinoline and 1-chloro-6-((5-methyl-1-((2-(trimethylsilyl)ethoxy)methyl)-1H-pyrazol-4-yl)methoxy)isoquinoline (0.180 g, 0.223 mmol), 6-chloropyridin-3-amine (0.0573 g, 0.446 mmol, methanesulfonato(2-di-t-butylphosphino-2′,4′,6′-tri-i-propyl-1,1′-biphenyl)(2′-amino-1,1′-biphenyl-2-yl)palladium(II) (0.035 g, 0.045 mmol), and cesium carbonate (0.290 g, 0.891 mmol) in 2-methylbutan-2-ol (3 mL) was stirred at 70° C. for 12 h. After cooling to ambient temperature, the reaction mixture was poured into water (30 mL). The mixture was extracted with ethyl acetate (3×30 mL). The combined organic phase was washed with brine (30 mL), dried over anhydrous sodium sulfate, and filtered. The filtrate was concentrated in vacuo and the obtained residue was purified by silica gel column chromatography, eluting with a gradient of 0 to 50% of ethyl acetate in petroleum ether, to provide a mixture of the title compounds as a yellowish oil (0.090 g, 41% yield): MS (ES+) m/z 496.2 (M+1), 498.2 (M+1).

### Step 2. Preparation of N-(6-chloropyridin-3-yl)-6-((3-methyl-1H-pyrazol-4-yl)methoxy)isoquinolin-1-amine

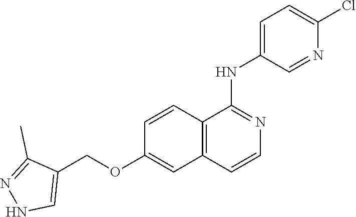 [SMILES: Cc1n[nH]cc1COc1ccc2c(Nc3ccc(Cl)nc3)nccc2c1]

A mixture of N-(6-chloropyridin-3-yl)-6-((3-methyl-1-((2-(trimethylsilyl)ethoxy)methyl)-1H-pyrazol-4-yl)methoxy)isoquinolin-1-amine and N-(6-chloropyridin-3-yl)-6-((5-methyl-1-((2-(trimethylsilyl)ethoxy)methyl)-1H-pyrazol-4-yl)methoxy)isoquinolin-1-amine (0.070 g, 0.141 mmol) and trifluoroacetic acid (0.7 mL, 9.45 mmol) in dichloromethane (3 mL) was stirred at ambient temperature for 12 h. The reaction mixture was carefully adjusted to pH 6 by the addition of solid sodium hydroxide and then reaction mixture was concentrated in vacuo. The residue was purified by reverse-phase preparative HPLC (Phenomenex Gemini-NX C18 75 mm×30 mm, 3 μm column), eluting with a gradient of 25% to 55% of acetonitrile in water (containing 10 mM of ammonium bicarbonate), to provide the title compound as a colorless solid (0.0068 g, 13% yield): ¹H NMR (400 MHz, CD₃OD) δ8.72 (d, J=2.6 Hz, 1H), 8.29-8.23 (m, 2H), 7.91 (d, J=5.8 Hz, 1H), 7.78-7.52 (m, 1H), 7.39 (d, J=8.8 Hz, 1H), 7.33 (d, J=2.4 Hz, 1H), 7.24 (dd, J=9.2, 2.4 Hz, 1H), 7.19 (d, J=5.8 Hz, 1H), 5.13 (s, 2H), 2.34 (s, 3H), two NH not observed; MS (ES+) m/z 366.1 (M+1), 368.1 (M+1).

### Example 394

### Synthesis of 3-(((1-((6-chloropyridin-3-yl)amino)isoquinolin-6-yl)oxy)methyl)oxetan-3-ol

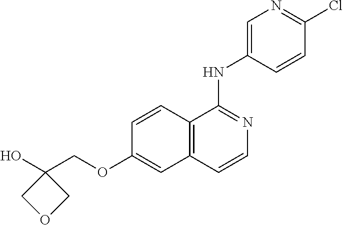 [SMILES: OC1(COc2ccc3c(Nc4ccc(Cl)nc4)nccc3c2)COC1]

### Step 1. Preparation of 3-(((1-chloroisoquinolin-6-yl)oxy)methyl)oxetan-3-ol

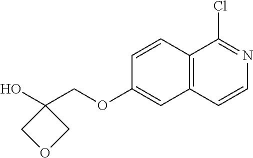 [SMILES: OC1(COc2ccc3c(Cl)nccc3c2)COC1]

To a mixture of 1,5-dioxaspiro[2.3]hexane (0.100 g, 1.16 mmol) and 1-chloroisoquinolin-6-01 (0.209 g, 1.16 mmol) in N,N-dimethylformamide (2 mL) was added potassium carbonate (0.482 g, 3.49 mmol), and the mixture was heated to 100° C. for 12 h. After cooling to ambient temperature, the mixture was diluted with ethyl acetate (20 mL) and washed with water (3×20 mL). The organic phase was dried over anhydrous sodium sulfate and filtered. The filtrate was concentrated in vacuo to provide a residue which was purified by silica gel column chromatography, eluting with 50 to 87% of ethyl acetate in petroleum ether, to afford the title compound as a colorless solid (0.070 g, 22% yield): ¹H NMR (400 MHz, DMSO-ds) δ8.21 (d, J=5.6 Hz, 1H), 8.18 (d, J=9.2 Hz, 1H), 7.76 (d, J=5.6 Hz, 1H), 7.56 (d, J=2.4 Hz, 1H), 7.46 (dd, J=2.4, 9.2 Hz, 1H), 6.15 (s, 1H), 4.65-4.47 (m, 4H), 4.32 (s, 2H); MS (ES+) m/z 266.1 (M+1), 268.1 (M+1).

### Step 2. Preparation of 3-(((1-((6-chloropyridin-3-yl)amino)isoquinolin-6-yl)oxy)methyl)oxetan-3-ol

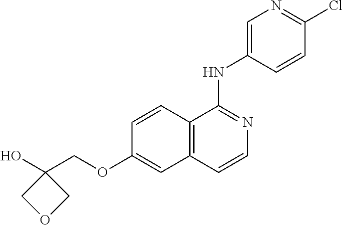 [SMILES: OC1(COc2ccc3c(Nc4ccc(Cl)nc4)nccc3c2)COC1]

Following the procedure as described or EXAMPLE 391, Step 3, and making variations as required to replace 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline with 3-(((1-chloroisoquinolin-6-yl)oxy)methyl)oxetan-3-ol, the title compound was obtained as a colorless solid (0.00480 g, 6% yield): ¹H NMR (400 MHz, DMSO-d₆) δ 9.44 (s, 1H), 8.88 (d, J=2.8 Hz, 1H), 8.46 (d, J=9.2 Hz, 1H), 8.42 (dd, J=2.8, 8.8 Hz, 1H), 7.96 (d, J=5.6 Hz, 1H), 7.46 (d, J=8.8 Hz, 1H), 7.39-7.30 (m, 2H), 7.20 (d, J=6.0 Hz, 1H), 6.11 (s, 1H), 4.62-4.49 (m, 4H), 4.29 (s, 2H); MS (ES+) m/z 358.2 (M+1), 360.2 (M+1).

### Example 395

### Synthesis of 6-((1H-pyrazol-4-yl)methoxy)-N-(2-methylpyrimidin-5-yl)isoquinolin-1-amine

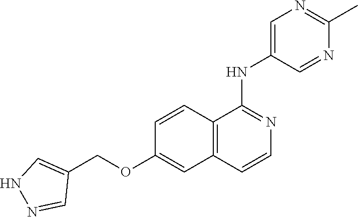 [SMILES: Cc1ncc(Nc2nccc3cc(OCc4cn[nH]c4)ccc23)cn1]

### Step 1. Preparation of N-(2-methylpyrimidin-5-yl)-6-((1-((2-(trimethylsilyl)ethoxy)methyl)-1H-pyrazol-4-yl)methoxy)isoquinolin-1-amine

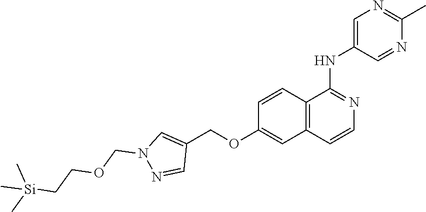

To a mixture of 2-methylpyrimidin-5-amine (0.168 g, 1.54 mmol), 1-chloro-6-((1-((2-(trimethylsilyl)ethoxy)methyl)-1H-pyrazol-4-yl)methoxy)isoquinoline (0.400 g, 1.03 mmol), sodium carbonate (0.326 g, 3.08 mmol), and (2′-amino-[1,1′-biphenyl]-2-yl)(dicyclohexyl(2′,6′-diisopropoxy-[1,1′-biphenyl]-2-yl)phosphoranyl)palladium(III) chloride (0.080 g, 0.103 mmol) was added 1,4-dioxane (10 mL) in a glove box. The resulting mixture was then heated to 90° C. for 12 h. After cooling to ambient temperature, the resulting mixture was poured into water (30 mL) and extracted with ethyl acetate (3×30 mL). The combined organic phase was washed with brine (30 mL), dried over anhydrous sodium sulfate, and filtered. The filtrate was concentrated in vacuo to afford a residue which was purified by reverse-phase column chromatography, eluting with acetonitrile in water (containing formic acid), to provide the title compound as a yellowish solid (0.450 g, 45% yield): ¹H NMR (400 MHz, CDCl₃) δ9.05 (s, 2H), 8.03 (d, J=5.8 Hz, 1H), 7.89 (d, J=9.4 Hz, 1H), 7.69 (d, J=13.6 Hz, 2H), 7.24 (dd, J=2.4, 9.2 Hz, 1H), 7.17-7.11 (m, 2H), 7.07-6.89 (m, 1H), 5.44 (s, 2H), 5.14 (s, 2H), 3.63-3.55 (m, 2H), 2.73 (s, 3H), 0.95-0.88 (m, 2H), −0.02 (s, 9H); MS (ES+) m/z 463.1 (M+1).

### Step 2. Preparation of 6-((1H-pyrazol-4-yl)methoxy)-N-(2-methylpyrimidin-5-yl)isoquinolin-1-amine hydrochloride

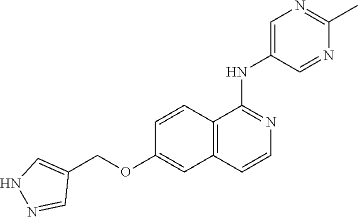 [SMILES: Cc1ncc(Nc2nccc3cc(OCc4cn[nH]c4)ccc23)cn1]

A mixture of N-(2-methylpyrimidin-5-yl)-6-((1-((2-(trimethylsilyl)ethoxy)methyl)-1H-pyrazol-4-yl)methoxy)isoquinolin-1-amine (0.200 g, 0.432 mmol) and trifluoroacetic acid (1.0 mL, 13.5 mmol) in dichloromethane (10 mL) was stirred at ambient temperature for 12 h. The reaction mixture was concentrated in vacuo to afford a residue which was purified by preparative HPLC (Phenomenex Luna C18 150 mm×25 mm, 10 μm column), eluting with a gradient of 8 to 38% of acetonitrile in water (containing 0.225% of formic acid). The desired fractions were collected, followed by addition of two drops of hydrochloric acid solution (12 M). The mixture was lyophilized to afford the title compound as a colorless solid (0.235 g, 71% yield): ¹H NMR (400 MHz, DMSO-d₆) δ11.46 (s, 1H), 8.95 (s, 2H), 8.84 (d, J=9.2 Hz, 1H), 7.83 (s, 2H), 7.67-7.60 (m, 2H), 7.49 (dd, J=2.4, 9.4 Hz, 1H), 7.36 (d, J=6.8 Hz, 1H), 5.24 (s, 2H), 2.72 (s, 3H), NH not observed; MS (ES+) m/z 333.2 (M+1).

### Example 396

### Synthesis of 6-((3-methyl-1H-pyrazol-4-yl)methoxy)-N-(2-methylpyrimidin-5-yl)isoquinolin-1-amine

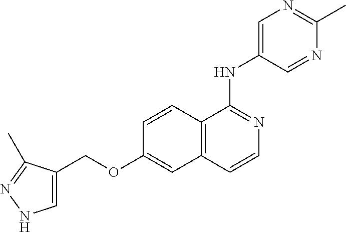 [SMILES: Cc1ncc(Nc2nccc3cc(OCc4c[nH]nc4C)ccc23)cn1]

### Step 1. Preparation of 6-((3-methyl-1-((2-(trimethylsilyl)ethoxy)methyl)-1H-pyrazol-4-yl)methoxy)-N-(2-methylpyrimidin-5-yl)isoquinolin-1-amine and 6-((5-methyl-1-((2-(trimethylsilyl)ethoxy)methyl)-1H-pyrazol-4-yl)methoxy)-N-(2-methylpyrimidin-5-yl)isoquinolin-1-amine

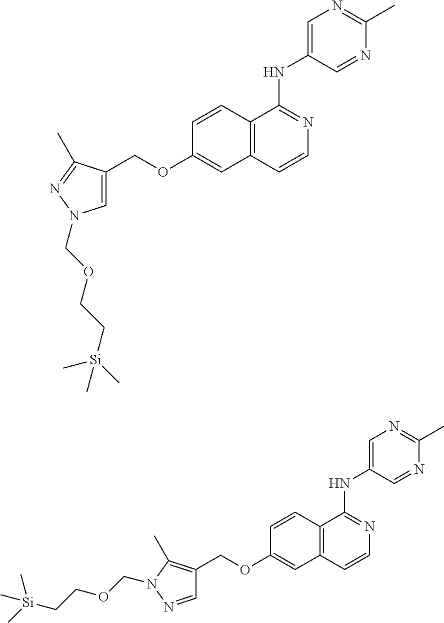

To a mixture of 1-chloro-6-((3-methyl-1-((2-(trimethylsilyl)ethoxy)methyl)-1H-pyrazol-4-yl)methoxy)isoquinoline and 1-chloro-6-((5-methyl-1-((2-(trimethylsilyl)ethoxy)methyl)-1H-pyrazol-4-yl)methoxy)isoquinoline (0.070 g, 0.087 mmol), 2-methylpyrimidin-5-amine (0.028 g, 0.260 mmol), and sodium carbonate (0.055 g, 0.519 mmol) in 1,4-dioxane (2 mL) was added chloro-(2-dicyclohexylphosphino-2,6-diisopropoxy-1,1-biphenyl)[2-(2-amino-1,1-biphenyl)]palladium(II) (0.014 g, 0.0173 mmol) in a glove box, and the mixture was heated to 90° C. for 12 h. After cooling to ambient temperature, the reaction mixture was filtered, and the filtrate was concentrated in vacuo. The residue was purified by reversed-phase column chromatography, eluting with acetonitrile in water (containing 0.1% of formic acid), to afford the title compound as a yellowish solid (0.700 g, 85% yield).

### Step 2. Preparation of 6-((3-methyl-1H-pyrazol-4-yl)methoxy)-N-(2-methylpyrimidin-5-yl)isoquinolin-1-amine

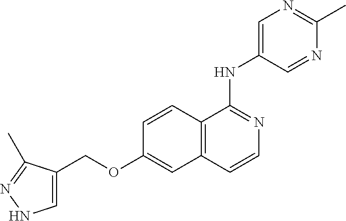 [SMILES: Cc1ncc(Nc2nccc3cc(OCc4c[nH]nc4C)ccc23)cn1]

Following the procedure as described for EXAMPLE 391, Step 4, and making variations as required to replace the mixture of N-(6-chloropyridin-3-yl)-6-((1-((2-(trimethylsilyl)ethoxy)methyl)-1H-pyrazol-3-yl)methoxy)isoquinolin-1-amine and N-(6-chloropyridin-3-yl)-6-((1-((2-(trimethylsilyl)ethoxy)methyl)-1H-pyrazol-5-yl)methoxy)isoquinolin-1-amine with a mixture of 6-((3-methyl-1-((2-(trimethylsilyl)ethoxy)methyl)-1H-pyrazol-4-yl)methoxy)-N-(2-methylpyrimidin-5-yl)isoquinolin-1-amine and 6-((5-methyl-1-((2-(trimethylsilyl)ethoxy)methyl)-1H-pyrazol-4-yl)methoxy)-N-(2-methylpyrimidin-5-yl)isoquinolin-1-amine, the title compound was obtained as a colorless solid (0.053 g, 80% yield): ¹H NMR (400 MHz, DMSO-d₆) δ 12.77-12.37 (m, 1H), 9.29 (s, 1H), 9.16 (s, 2H), 8.41 (d, J=9.2 Hz, 1H), 7.94 (d, J=5.6 Hz, 1H), 7.40 (d, J=2.4 Hz, 1H), 7.27 (dd, J=2.4, 9.2 Hz, 1H), 7.17 (d, J=5.6 Hz, 1H), 5.08 (s, 2H), 2.57 (s, 3H), 2.25 (s, 3H), NH not observed; MS (ES+) m/z 347.1 (M+1).

### Example 397

### Synthesis of N-(6-chloropyridin-3-yl)-6-((4-fluoro-1-methyl-1H-pyrazol-3-yl)methoxy)isoquinolin-1-amine formate

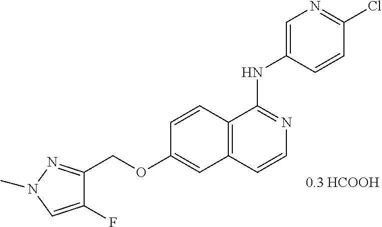 [SMILES: Cn1cc(F)c(COc2ccc3c(Nc4ccc(Cl)nc4)nccc3c2)n1]

### Step 1. Preparation of 3-(chloromethyl)-4-fluoro-1-methyl-1H-pyrazole

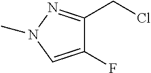

Following the procedure as described for EXAMPLE 392, Step 1, and making variations as required to replace (5-methyl-1H-pyrazol-3-yl)methanol with (4-fluoro-1-methyl-1H-pyrazol-3-yl)methanol, the title compound was obtained as a light yellow oil (0.300 g, quantitative yield): ¹H NMR (400 MHz, CDCl₃) δ7.25 (d, J=4.8 Hz, 1H), 4.60 (s, 2H), 3.85 (s, 3H).

### Step 2. Preparation of 1-chloro-6-((4-fluoro-1-methyl-1H-pyrazol-3-yl)methoxy)isoquinoline

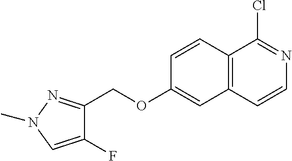 [SMILES: Cn1cc(F)c(COc2ccc3c(Cl)nccc3c2)n1]

Following the procedure as described for EXAMPLE 391, Step 1, and making variations as required to replace 3-(chloromethyl)-1H-pyrazole hydrochloride with 3-(chloromethyl)-4-fluoro-1-methyl-1H-pyrazole, the title compound was obtained as a colorless solid (0.140 g, 86% yield): ¹H NMR (400 MHz, DMSO-d₆) δ 8.22 (d, J=6.0 Hz, 1H), 8.17 (d, J=9.2 Hz, 1H), 7.89 (d, J=4.8 Hz, 1H), 7.76 (d, J=6.0 Hz, 1H), 7.65 (d, J=2.4 Hz, 1H), 7.47-7.41 (m, 1H), 5.21 (s, 2H), 3.80 (s, 3H).

### Step 3. Preparation of N-(6-chloropyridin-3-yl)-6-((4-fluoro-1-methyl-1H-pyrazol-3-yl)methoxy)isoquinolin-1-amine formate

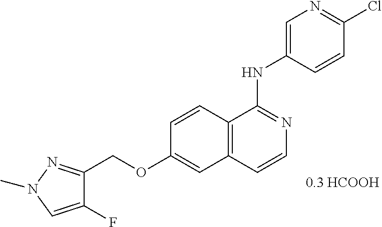 [SMILES: Cn1cc(F)c(COc2ccc3c(Nc4ccc(Cl)nc4)nccc3c2)n1]

Following the procedure as described for EXAMPLE 391, Step 3, and making variations as required to replace 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline with 1-chloro-6-((4-fluoro-1-methyl-1H-pyrazol-3-yl)methoxy)isoquinoline, the title compound were obtained as a colorless solid (0.026 g, 13% yield): ¹H NMR (400 MHz, DMSO-d₆) δ9.38 (s, 1H), 8.87 (d, J=2.8 Hz, 1H), 8.48-8.36 (m, 2H), 8.26 (s, 0.3H), 7.97 (d, J=6.0 Hz, 1H), 7.88 (d, J=4.8 Hz, 1H), 7.50-7.38 (m, 2H), 7.34-7.24 (m, 1H), 7.17 (d, J=6.0 Hz, 1H), 5.17 (s, 2H), 3.80 (s, 3H), COOH not observed; MS (ES+) m/z 384.1 (M+1), 386.1 (M+1).

### Example 398

### Synthesis of N-(6-chloropyridin-3-yl)-3-methylisoquinolin-1-amine

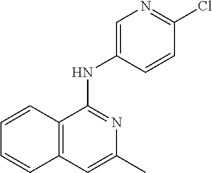 [SMILES: Cc1cc2ccccc2c(Nc2ccc(Cl)nc2)n1]

### Step 1. Preparation of 3-methylisoquinoline 2-oxide

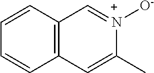

Following the procedure as described for EXAMPLE 10, Step 1 and making variations as required to replace methyl isoquinoline-6-carboxylate with 3-methylisoquinoline, the title compound was obtained as a colorless solid (0.700 g, 70% yield): ¹H NMR (400 MHz, CDCl₃) δ 8.87 (s, 1H), 7.72-7.70 (m, 2H), 7.66 (s, 1H), 7.57-7.55 (m, 2H), 2.67 (s, 3H).

### Step 2. Preparation of 1-chloro-3-methylisoquinoline

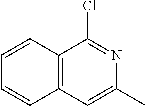

Following the procedure as described for EXAMPLE 10, Step 2 and making variations as required to replace 6-(methoxycarbonyl)isoquinoline 2-oxide with 3-methylisoquinoline 2-oxide, the title compound was obtained as a yellow solid (0.500 g, 64% yield): MS (ES+) m/z 178.1 (M+1), 180.1 (M+1).

### Step 3. Preparation of N-(6-chloropyridin-3-yl)-3-methylisoquinolin-1-amine

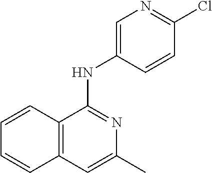 [SMILES: Cc1cc2ccccc2c(Nc2ccc(Cl)nc2)n1]

Following the procedure as described for EXAMPLE 391, Step 3, and making variations as required to replace 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline with 1-chloro-3-methylisoquinoline, the title compound was obtained as a yellow solid (0.017 g, 22% yield): ¹H NMR (400 MHz, CDCl₃) δ8.71 (d, J=2.8 Hz, 1H), 8.44 (dd, J=2.4, 8.6 Hz, 1H), 7.90 (d, J=8.4 Hz, 1H), 7.75-7.69 (m, 1H), 7.68-7.62 (m, 1H), 7.58-7.49 (m, 1H), 7.34 (d, J=8.6 Hz, 1H), 7.21-7.12 (m, 1H), 7.08 (s, 1H), 2.58 (s, 3H); MS (ES+) m/z 270.1 (M+1), 272.1 (M+1).

### Example 399

### Synthesis of N-(6-chloropyridin-3-yl)-3-methoxy-1,7-naphthyridin-8-amine

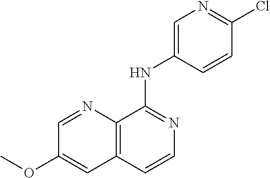 [SMILES: COc1cnc2c(Nc3ccc(Cl)nc3)nccc2c1]

Following the procedure as described for EXAMPLE 391, Step 3, and making variations as required to replace 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline with 8-chloro-3-methoxy-1,7-naphthyridine, the title compound was obtained as a yellow solid (0.107 g, 35% yield): ¹H NMR (400 MHz, DMSO-d₆) δ9.91 (s, 1H), 9.10 (dd, J=2.9, 0.6 Hz, 1H), 8.67-8.64 (m, 2H), 8.07 (d, J=5.7 Hz, 1H), 7.75 (d, J=2.8 Hz, 1H), 7.46 (d, J=8.7 Hz, 1H), 7.22 (d, J=5.8 Hz, 1H), 3.98 (s, 3H); MS (ES+) m/z 287.1 (M+1), 289.1 (M+1).

### Example 400

### Synthesis of 2-(4-(((1-((6-chloropyridin-3-yl)amino)isoquinolin-6-yl)oxy)methyl)-1H-pyrazol-1-yl)acetonitrile

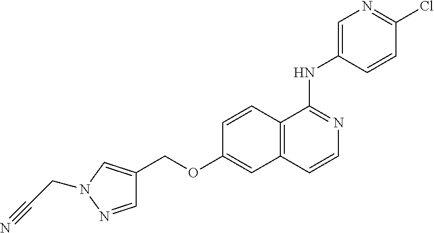 [SMILES: N#CCn1cc(COc2ccc3c(Nc4ccc(Cl)nc4)nccc3c2)cn1]

### Step 1. Preparation of tert-butyl 4-(((1-chloroisoquinolin-6-yl)oxy)methyl)-1H-pyrazole-1-carboxylate

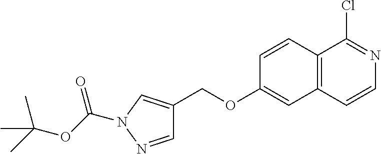 [SMILES: CC(C)(C)OC(=O)n1cc(COc2ccc3c(Cl)nccc3c2)cn1]

Following the procedure as described for EXAMPLE 1, Step 1 and making variations as required to replace 2-propanol with tert-butyl 4-(hydroxymethyl)-1H-pyrazole-1-carboxylate, the title compound was obtained as a brownish oil (0.450 g, 50% yield): ¹H NMR (400 MHz, DMSO-d₆) δ 8.48 (s, 1H), 8.22 (d, J=5.6 Hz, 1H), 8.18 (d, J=9.2 Hz, 1H), 7.97 (s, 1H), 7.80 (d, J=5.6 Hz, 1H), 7.63 (d, J=2.4 Hz, 1H), 7.45 (dd, J=2.4, 9.2 Hz, 1H), 5.22 (s, 2H), 1.58 (s, 9H).

### Step 2. Preparation of 6-((1H-pyrazol-4-yl)methoxy)-1-chloroisoquinoline

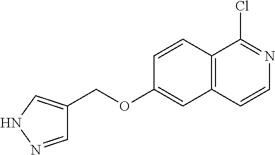 [SMILES: Clc1nccc2cc(OCc3cn[nH]c3)ccc12]

To a solution of tert-butyl 4-(((1-chloroisoquinolin-6-yl)oxy)methyl)-1H-pyrazole-1-carboxylate (0.450 g, 1.25 mmol) in dichloromethane (5 mL) was added trifluoroacetic acid (0.50 mL, 6.73 mmol). The mixture was stirred ambient temperature for 16 h and then concentrated in vacuo. The residue was diluted with ethyl acetate (10 mL) and adjusted to pH=7 with saturated sodium bicarbonate solution. The organic phase was separated, and the aqueous phase was extracted with ethyl acetate (3×30 mL). The combined organic layers were washed with brine (3×30 mL), dried over anhydrous sodium sulfate, and filtered. The filtrate was concentrated in vacuo to give a residue which was purified by silica gel column chromatography, eluting with a gradient of 0 to 68% of ethyl acetate in petroleum ether, to afford the title compound as a colorless solid (0.320 g, 99% yield): ¹H NMR (400 MHz, DMSO-d₆) δ 12.90 (s, 1H), 8.21 (d, J=5.6 Hz, 1H), 8.16 (d, J=9.2 Hz, 1H), 7.94 (br s, 1H), 7.78 (d, J=5.6 Hz, 1H), 7.66-7.61 (m, 2H), 7.41 (dd, J=2.4, 9.2 Hz, 1H), 5.18 (s, 2H).

### Step 3. Preparation of 2-(4-(((1-chloroisoquinolin-6-yl)oxy)methyl)-1H-pyrazol-1-yl)acetonitrile

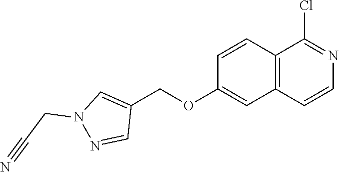 [SMILES: N#CCn1cc(COc2ccc3c(Cl)nccc3c2)cn1]

To a solution of 6-((1H-pyrazol-4-yl)methoxy)-1-chloroisoquinoline (0.050 g, 0.193 mmol) in N,N-dimethyl formamide (1.0 mL) was added potassium carbonate (0.080 g, 0.579 mmol) and 2-bromoacetonitrile (0.030 mL, 0.450 mmol), and the mixture was stirred at ambient temperature for 16 h. The reaction mixture was diluted with water (10 mL) and extracted with ethyl acetate (3×10 mL). The combined organic phase was washed with brine (3×10 mL), dried over anhydrous sodium sulfate, and filtered. The filtrate was concentrated in vacuo to give a residue which was purified by silica gel column chromatography, eluting with a gradient of 0 to 50% of ethyl acetate in petroleum ether, to provide the title compound as a yellowish oil (0.040 g, 70% yield): ¹H NMR (400 MHz, DMSO-d₆) δ 8.22 (d, J=5.6 Hz, 1H), 8.17 (d, J=9.2 Hz, 1H), 8.04 (s, 1H), 7.79-7.76 (m, 2H), 7.62 (d, J=2.4 Hz, 1H), 7.43 (dd, J=2.4, 9.2 Hz, 1H), 5.50 (s, 2H), 5.19 (s, 2H).

### Step 4. Preparation of 2-(4-(((1-((6-chloropyridin-3-yl)amino)isoquinolin-6-yl)oxy)methyl)-1H-pyrazol-1-yl)acetonitrile

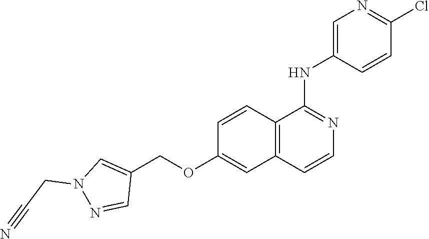 [SMILES: N#CCn1cc(COc2ccc3c(Nc4ccc(Cl)nc4)nccc3c2)cn1]

Following the procedure as described for EXAMPLE 391, Step 3, and making variations as required to replace 1-chloro-6-((1-((2-(trimethylsilyl)ethoxy)methyl)-1H-pyrazol-3-yl)methoxy)isoquinoline and 1-chloro-6-((1-((2-(trimethylsilyl)ethoxy)methyl)-1H-pyrazol-5-yl)methoxy)isoquinoline with 2-(4-(((1-chloroisoquinolin-6-yl)oxy)methyl)-1H-pyrazol-1-yl)acetonitrile, the title compound was obtained as a colorless solid (0.007 g, 12% yield): ¹H NMR (400 MHz, DMSO-d₆) δ 9.37 (s, 1H), 8.88 (d, J=2.8 Hz, 1H), 8.45-8.40 (m, 2H), 8.03 (s, 1H), 7.97 (d, J=5.6 Hz, 1H), 7.76 (s, 1H), 7.44 (d, J=8.8 Hz, 1H), 7.41 (d, J=2.4 Hz, 1H), 7.28 (dd, J=2.4, 9.2 Hz, 1H), 7.20 (d, J=6.0 Hz, 1H), 5.51 (s, 2H), 5.15 (s, 2H); MS (ES+) m/z 391.0 (M+1), 393.0 (M+1).

### Example 401

### Synthesis of N-(1-((6-chloropyridin-3-yl)amino)isoquinolin-6-yl)-1-(hydroxymethyl)cyclopropane-1-carboxamide

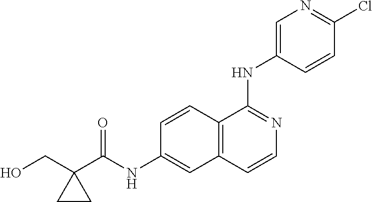 [SMILES: O=C(Nc1ccc2c(Nc3ccc(Cl)nc3)nccc2c1)C1(CO)CC1]

### Step 1. Preparation of N-(1-((6-chloropyridin-3-yl)((2-(trimethylsilyl)ethoxy)methyl)amino)isoquinolin-6-yl)-1-(hydroxymethyl)cyclopropane-1-carboxamide

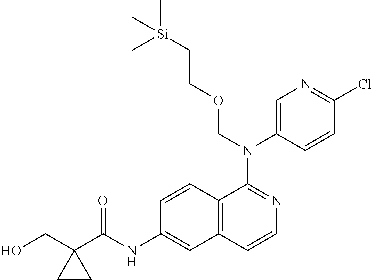 [SMILES: C[Si](C)(C)CCOCN(c1ccc(Cl)nc1)c1nccc2cc(NC(=O)C3(CO)CC3)ccc12]

To a solution of 1-(hydroxymethyl)cyclopropane-1-carboxamide (0.043 g, 0.373 mmol) and N-(6-chloro-3-pyridyl)-6-fluoro-N-(2-trimethylsilylethoxymethyl)isoquinolin-1-amine (0.050 g, 0.124 mmol) in tetrahydrofuran (2 mL) was added sodium hydride (60% dispersion in mineral oil, 0.010 g, 0.248 mmol) at 0° C. The reaction mixture was allowed to warm to ambient temperature and then heated to 60° C. for 16 h. The mixture was quenched by addition of water (10 mL). The mixture was extracted with ethyl acetate (3×10 mL) and the combined organic phase was washed with brine (20 mL), dried over anhydrous sodium sulfate, and filtered. The filtrate was concentrated in vacuo and the obtained residue was purified by silica gel column chromatography, eluting with a gradient of 0% to 100% of ethyl acetate in petroleum ether, to provide the title compound as a colorless oil (0.010 g, 22% yield): ¹H NMR (400 MHz, DMSO-d₆) δ9.90 (s, 1H), 8.39 (s, 1H), 8.29 (s, 1H), 7.92 (d, J=2.8 Hz, 1H), 7.71-7.64 (m, 2H), 7.63-7.58 (m, 1H), 7.30 (d, J=8.8 Hz, 1H), 7.15 (dd, J=8.8, 2.8 Hz, 1H), 5.48 (t, J=5.2 Hz, 1H), 5.36 (s, 2H), 3.73-3.61 (m, 2H), 3.54 (t, J=8.0 Hz, 2H), 1.12-1.06 (m, 2H), 0.87-0.73 (m, 4H), −0.14 (s, 9H).

### Step 2. Preparation of N-(1-((6-chloropyridin-3-yl)amino)isoquinolin-6-yl)-1-(hydroxymethyl)cyclopropane-1-carboxamide

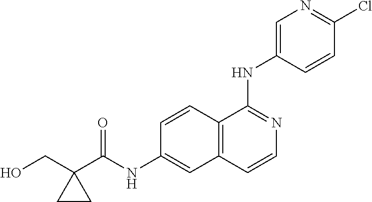 [SMILES: O=C(Nc1ccc2c(Nc3ccc(Cl)nc3)nccc2c1)C1(CO)CC1]

To a solution of N-(1-((6-chloropyridin-3-yl)((2-(trimethylsilyl)ethoxy)methyl)amino)isoquinolin-6-yl)-1-(hydroxymethyl)cyclopropane-1-carboxamide (0.010 g, 0.020 mmol) in dichloromethane (1 mL) was added trifluoroacetic acid (0.307 g, 2.69 mmol). The reaction mixture was stirred at ambient temperature for 16 h and then concentrated in vacuo. The residue was purified by reverse-phase preparative HPLC (Waters XBridge 150 mm×25 mm, 5 μm column), eluting with a gradient of 25% to 55% of acetonitrile in water (containing 0.1% of ammonium hydroxide), to afford the title compound as a colorless solid (0.0020 g, 27% yield): ¹H NMR (400 MHz, DMSO-d₆) δ 9.88 (s, 1H), 9.42 (s, 1H), 8.89 (d, J=2.8 Hz, 1H), 8.56-8.36 (m, 2H), 8.20 (d, J=2.0 Hz, 1H), 7.97 (d, J=5.6 Hz, 1H), 7.76 (dd, J=9.2, 2.0 Hz, 1H), 7.46 (d, J=8.8 Hz, 1H), 7.19 (d, J=6.0 Hz, 1H), 5.54 (t, J=5.2 Hz, 1H), 3.71 (d, J=5.2 Hz, 2H), 1.17-1.06 (m, 2H), 0.90-0.69 (m, 2H); MS (ES+) m/z 369.1 (M+1), 371.1 (M+1).

### Example 402

### Synthesis of NF-(6-chloropyridin-3-yl)-N⁴-(2,2,2-trifluoroethyl)-1H-pyrrolo[2,3-c]pyridine-4,7-diamine

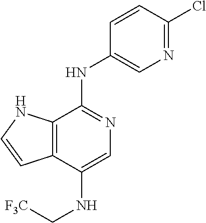 [SMILES: FC(F)(F)CNc1cnc(Nc2ccc(Cl)nc2)c2[nH]ccc12]

### Step 1. Preparation of (E)-2-(5-bromo-2-methoxy-3-nitropyridin-4-yl)-N,N-dimethylethen-1-amine

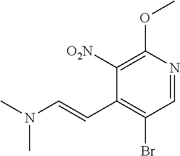

To a solution of 5-bromo-2-methoxy-4-methyl-3-nitropyridine (10.0 g, 40.5 mmol) in N,N-dimethylformamide (100 mL) was added 1,1-dimethoxy-N,N-dimethylmethanamine (41.0 g, 344 mmol) dropwise at 85° C. The reaction mixture was stirred at 95° C. for 7 h. After cooling to ambient temperature, the reaction mixture was poured into ice-water (300 mL). The residue was filtered, and the filter cake was washed with water (2×50 mL) and dried in vacuo to provide the title compound as a red solid (12.0 g, 98% yield): ¹H NMR (400 MHz, DMSO-d₆) δ8.23 (s, 1H), 7.05 (d, J=13.6 Hz, 1H), 4.80 (d, J=13.6 Hz, 1H), 3.88 (s, 3H), 2.90 (s, 6H).

### Step 2. Preparation of 4-bromo-7-methoxy-1H-pyrrolo[2,3-c]pyridine

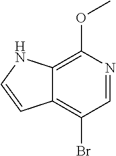

A mixture of (E)-2-(5-bromo-2-methoxy-3-nitropyridin-4-yl)-N,N-dimethylethen-1-amine (12.0 g, 39.7 mmol), iron powder (12.0 g, 215 mmol), and ammonium chloride (12.0 g, 224 mmol) in methanol (450 mL) and water (60 mL) was stirred at 90° C. for 12 h. The mixture was filtered, and the filter cake was washed with methanol (3×40 mL). The combined filtrate was concentrated in vacuo and the obtained residue was purified by silica gel column chromatography, eluting with a gradient of 15 to 20% of ethyl acetate in petroleum ether, to provide the title compound as a yellow solid (7.00 g, 72% yield): ¹H NMR (400 MHz, DMSO-d₆) d12.15 (br s, 1H), 7.75 (s, 1H), 7.55 (t, J=2.8 Hz, 1H), 6.46-6.38 (m, 1H), 4.01 (s, 3H).

### Step 3. Preparation of 7-methoxy-N-(2,2,2-trifluoroethyl)-1H-pyrrolo[2,3-c]pyridin-4-amine

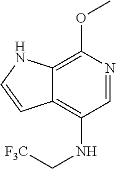 [SMILES: COc1ncc(NCC(F)(F)F)c2cc[nH]c12]

To a mixture of 4-bromo-7-methoxy-1H-pyrrolo[2,3-c]pyridine (2.00 g, 8.81 mmol), [(2-di-tert-butylphosphino-2′,4′,6′-triisopropyl-1,1′-biphenyl)-2-(2′-amino-1,1′-biphenyl)] palladium(II) methanesulfonate (0.700 g, 0.881 mmol) and sodium 2-methylpropan-2-olate (4.23 g, 44.0 mmol) in 1,4-dioxane (20 mL) was added 2,2,2-trifluoroethanamine (0.873 g, 8.81 mmol) in a glove box. The mixture was heated to 90° C. and stirred for 12 h. After cooling to ambient temperature, the reaction mixture was poured into water (50 mL). The mixture was extracted with ethyl acetate (3×50 mL). The combined organic phase was washed with brine (30 mL), dried over anhydrous sodium sulfate, and filtered. The filtrate was concentrated in vacuo and the obtained residue was purified by silica gel column chromatography, eluting with 25% of ethyl acetate in petroleum ether, to provide the title compound as a dark oil (2.10 g, 88% yield): ¹H NMR (400 MHz, CDCl₃) δ8.82 (s, 1H), 7.24 (t, J=2.8 Hz, 1H), 7.20 (s, 1H), 6.50 (dd, J=2.4, 2.8 Hz, 1H), 4.05 (s, 3H), 3.87 (q, J=9.0 Hz, 2H), NH not observed; MS (ES+) m/z 246.1 (M+1).

### Step 4. Preparation of 7-chloro-N-(2,2,2-trifluoroethyl)-1H-pyrrolo[2,3-c]pyridin-4-amine

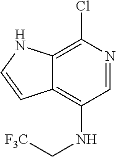 [SMILES: FC(F)(F)CNc1cnc(Cl)c2[nH]ccc12]

A mixture of 7-methoxy-N-(2,2,2-trifluoroethyl)-1H-pyrrolo[2,3-c]pyridin-4-amine (2.10 g, 8.56 mmol) in phosphoryl trichloride (34.7 g, 226 mmol) was stirred at 100° C. for 12 h. After cooling to ambient temperature, the reaction mixture was slowly poured into water (50 mL). The mixture was adjusted to pH=7-8 with sodium carbonate and then extracted with ethyl acetate (3×50 mL). The combined organic phase was washed with brine (30 mL), dried over anhydrous sodium sulfate, and filtered. The filtrate was concentrated in vacuo and the obtained residue was purified by silica gel column chromatography, eluting with 25% of ethyl acetate in petroleum ether, to provide the title compound as a brownish solid (0.790 g, 31% yield): ¹H NMR (400 MHz, DMSO-d₆) δ 11.73 (s, 1H), 7.44 (t, J=2.8 Hz, 1H), 7.36 (s, 1H), 6.81 (dd, J=2.2, 2.8 Hz, 1H), 6.50 (t, J=6.8 Hz, 1H), 4.15-4.02 (m, 2H).

### Step 5. Preparation of tert-butyl (7-chloro-1H-pyrrolo[2,3-c]pyridin-4-yl)(2,2,2-trifluoroethyl)carbamate

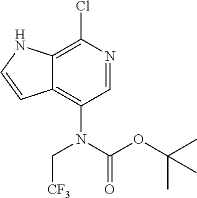 [SMILES: CC(C)(C)OC(=O)N(CC(F)(F)F)c1cnc(Cl)c2[nH]ccc12]

A mixture of 7-chloro-N-(2,2,2-trifluoroethyl)-1H-pyrrolo[2,3-c]pyridin-4-amine (0.790 g, 3.16 mmol), triethylamine (0.961 g, 9.49 mmol), and di-tert-butyl dicarbonate (1.04 g, 4.75 mmol) in dichloromethane (10 mL) was stirred at 50° C. for 12 h. After cooling to ambient temperature, the reaction mixture was concentrated in vacuo. The residue was purified by silica gel column chromatography, eluting with 25% of ethyl acetate in petroleum ether, to provide the title compound as a colorless solid (0.870 g, 78% yield): ¹H NMR (400 MHz, DMSO-d₆) δ7.77 (d, J=3.8 Hz, 1H), 7.70 (s, 1H), 7.05 (d, J=3.8 Hz, 1H), 6.77 (t, J=6.8 Hz, 1H), 4.15 (dd, J=7.0, 9.4 Hz, 2H), 1.60 (s, 9H).

### Step 6. Preparation of N-(6-chloropyridin-3-yl)-N⁴-(2,2,2-trifluoroethyl)-1H-pyrrolo[2,3-c]pyridine-4,7-diamine

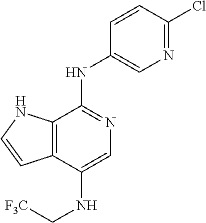 [SMILES: FC(F)(F)CNc1cnc(Nc2ccc(Cl)nc2)c2[nH]ccc12]

To a mixture of tert-butyl (7-chloro-1H-pyrrolo[2,3-c]pyridin-4-yl)(2,2,2-trifluoroethyl)carbamate (0.200 g, 0.572 mmol), 6-chloropyridin-3-amine (0.074 g, 0.572 mmol), and cesium carbonate (0.373 g, 1.14 mmol) in 2-methylbutan-2-ol (6 mL) was added [(2-di-tert-butylphosphino-2′,4′,6′-triisopropyl-1,1′-biphenyl)-2-(2′-amino-1,1′-biphenyl)] palladium(II) methanesulfonate (0.045 g, 0.057 mmol). The mixture was heated to 90° C. and stirred for 12 h. After cooling to ambient temperature, the reaction mixture was poured into water (50 mL). The mixture was extracted with ethyl acetate (6×30 mL). The combined organic phase was washed with brine (30 mL), dried over anhydrous sodium sulfate, and filtered. The filtrate was concentrated in vacuo and the obtained residue was purified by preparative reverse-phase HPLC (Phenomenex Luna C18 150 mm×25 mm, 10 μm column), eluting with a gradient of 3% to 33% of acetonitrile in water (containing 0.225% of formic acid), to provide the title compound as a yellowish solid (0.114 g, 11% yield): ¹H NMR (400 MHz, DMSO-d₆) δ11.13 (s, 1H), 8.69 (s, 1H), 8.63 (d, J=2.8 Hz, 1H), 8.29 (dd, J=8.8, 2.8 Hz, 1H), 7.44 (t, J=2.6 Hz, 1H), 7.35 (d, J=8.8 Hz, 1H), 7.22 (s, 1H), 6.72-6.67 (m, 1H), 5.93 (t, J=6.8 Hz, 1H), 4.06-3.93 (m, 2H); MS (ES+) m/z 342.1 (M+1), 344.1 (M+1).

### Example 403

### Synthesis of N⁴-benzyl-N⁷-(6-chloropyridin-3-yl)-1H-pyrrolo[2,3-c]pyridine-4,7-diamine

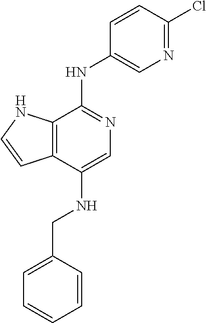 [SMILES: Clc1ccc(Nc2ncc(NCc3ccccc3)c3cc[nH]c23)cn1]

### Step 1. Preparation of N-benzyl-7-methoxy-1H-pyrrolo[2,3-c]pyridin-4-amine

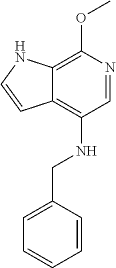 [SMILES: COc1ncc(NCc2ccccc2)c2cc[nH]c12]

Following the procedure as described for EXAMPLE 402, Step 3, and making variations as required to replace 2,2,2-trifluoroethanamine with phenylmethanamine, the title compound was obtained as a yellowish oil (1.00 g, 90% yield): ¹H NMR (400 MHz, DMSO-d₆) δ 11.46 (br s, 1H), 7.40 (d, J=7.2 Hz, 2H), 7.34-7.27 (m, 2H), 7.25 (t, J=2.8 Hz, 1H), 7.23-7.18 (m, 1H), 6.71-6.63 (m, 2H), 5.92 (t, J=6.0 Hz, 1H), 4.36 (d, J=6.0 Hz, 2H), 3.84 (s, 3H), one NH not observed.

### Step 2. Preparation of N-benzyl-7-chloro-1H-pyrrolo[2,3-c]pyridin-4-amine

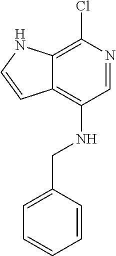 [SMILES: Clc1ncc(NCc2ccccc2)c2cc[nH]c12]

Following the procedure as described for EXAMPLE 402, Step 4, and making variations as required to replace 7-methoxy-N-(2,2,2-trifluoroethyl)-1H-pyrrolo[2,3-c]pyridin-4-amine with N-benzyl-7-methoxy-1H-pyrrolo[2,3-c]pyridin-4-amine, the title compound was obtained as a yellow solid (0.700 g, 69% yield): ¹H NMR (400 MHz, DMSO-d₆) δ11.62 (br s, 1H), 7.44-7.36 (m, 3H), 7.35-7.27 (m, 2H), 7.26-7.19 (m, 1H), 6.99 (s, 1H), 6.84-6.79 (m, 1H), 6.77-6.50 (m, 1H), 4.44 (s, 2H).

### Step 3. Preparation of N⁴-benzyl-N⁷-(6-chloropyridin-3-yl)-1H-pyrrolo[2,3-c]pyridine-4,7-diamine

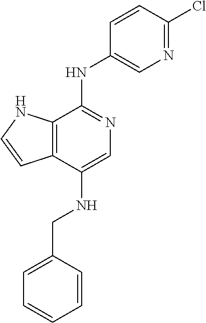 [SMILES: Clc1ccc(Nc2ncc(NCc3ccccc3)c3cc[nH]c23)cn1]

Following the procedure as described for EXAMPLE 402, Step 6 and making variations as required to replace tert-butyl (7-chloro-1H-pyrrolo[2,3-c]pyridin-4-yl)(2,2,2-trifluoroethyl)carbamate with N-benzyl-7-chloro-1H-pyrrolo[2,3-c]pyridin-4-amine, the title compound was obtained as a yellow solid (0.069 g, 16% yield): ¹H NMR (400 MHz, DMSO-d₆) δ 11.04 (br s, 1H), 8.65-8.51 (m, 2H), 8.31-8.19 (m, 1H), 7.47-7.37 (m, 3H), 7.36-7.26 (m, 3H), 7.25-7.17 (m, 1H), 6.88 (s, 1H), 6.77-6.69 (m, 1H), 6.05 (t, J=6.0 Hz, 1H), 4.40 (d, J=6.0 Hz, 2H); MS (ES+) m/z 350.1 (M+1), 352.1 (M+1).

### Example 404

### Synthesis of N-(6-chloropyridin-3-yl)-6-((2,2-difluorocyclopropyl)methoxy)isoquinolin-1-amine

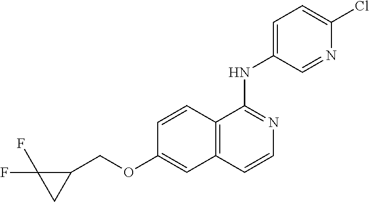 [SMILES: FC1(F)CC1COc1ccc2c(Nc3ccc(Cl)nc3)nccc2c1]

### Step 1. Preparation of 1-chloro-6-((2,2-difluorocyclopropyl)methoxy)isoquinoline

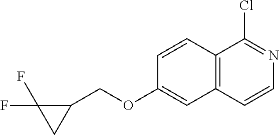 [SMILES: FC1(F)CC1COc1ccc2c(Cl)nccc2c1]

To a solution of 1-chloroisoquinolin-6-ol (0.250 g, 1.39 mmol), (2,2-difluorocyclopropyl) methanol (0.226 g, 2.09 mmol), and triphenylphosphine (0.548 g, 2.09 mmol) in tetrahydrofuran (14 mL) was added diisopropyl azodicarboxylate (0.41 mL, 2.09 mmol) dropwise at 0° C. The reaction mixture was allowed to warm to ambient temperature and stirred for 24 h, and then concentrated in vacuo. The residue was purified by silica gel column chromatography, eluting with a gradient of 0% to 30% of ethyl acetate in heptane, to afford the title compound as a colorless solid (0.320 g, 85% yield): ¹H NMR (300 MHz, CDCl₃) δ8.27-8.22 (m, 1H), 8.20 (d, J=5.7 Hz, 1H), 7.49-7.45 (m, 1H), 7.32 (dd, J=9.3, 2.5 Hz, 1H), 7.07 (d, J=2.5 Hz, 1H), 4.28-4.12 (m, 2H), 2.25-2.08 (m, 1H), 1.75-1.61 (m, 1H), 1.42-1.27 (m, 1H); MS (ES+) m/z 270.0 (M+1), 272.0 (M+1).

### Step 2. N-(6-chloropyridin-3-yl)-6-(2,2-difluorocyclopropyl)methoxy)isoquinolin-1-amine

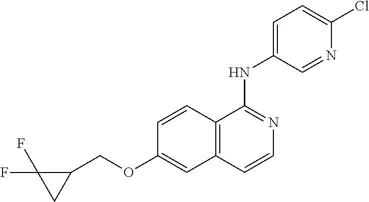 [SMILES: FC1(F)CC1COc1ccc2c(Nc3ccc(Cl)nc3)nccc2c1]

A mixture of 1-chloro-6-((2,2-difluorocyclopropyl)methoxy)isoquinoline (0.150 g, 0.556 mmol), 6-chloropyridin-3-amine (0.072 g, 0.556 mmol), and potassium phosphate tribasic (0.352 g, 1.67 mmol) in anhydrous 1,2-dimethoxyethane (7 mL) was purged with argon for 20 minutes, and then 2-dicyclohexylphosphino-2′,4′,6′-triisopropylbiphenyl (0.027 g, 0.056 mmol) was added to it, followed by tris(dibenzylideneacetone)dipalladium(0) (0.026 g, 0.028 mmol). The mixture was purged with argon for additional 5 minutes and then heated to 110° C. for 16 h. The reaction mixture was allowed to cool to ambient temperature and filtered through a pad of diatomaceous earth. The pad was washed with ethyl acetate (2×20 mL) and the combined filtrate was concentrated in vacuo. The residue was purified by silica gel column chromatography, eluting with a gradient of 0% to 50% of ethyl acetate in heptane to afford the title compound as a colorless solid (0.045 g, 22% yield): ¹H NMR (500 MHz, DMSO-d₆) δ9.39 (s, 1H), 8.91-8.86 (m, 1H), 8.50-8.39 (m, 2H), 7.97 (d, J=5.8 Hz, 1H), 7.48-7.41 (m, 1H), 7.36-7.28 (m, 2H), 7.18 (d, J=5.7 Hz, 1H), 4.39-4.27 (m, 1H), 4.21-4.08 (m, 1H), 2.41-2.21 (m, 1H), 1.86-1.70 (m, 1H), 1.62-1.46 (m, 1H); MS (ES+) m/z 362.2 (M+1), 364.2 (M+1).

### Examples 405-417

In a similar manner as described in EXAMPLE 404, utilizing the appropriately substituted starting materials and intermediates, the following compounds were prepared:

| Example No. | Name | MS (ES+) m/z | NMR |
|---|---|---|---|
| 405 | (S)-N-(6-chloropyridin-3- | 384.2 (M + 1), | ¹H NMR (500 MHZ, DMSO- |
| | yl)-6-((5,5-dimethyltetra- | 386.2 (M + 1) | d₆) δ 9.37 (s, 1H), 8.89-8.86 |
| | hydrofuran-2- | | (m, 1H), 8.45-8.40 (m, 2H), |
| | yl)methoxy)isoquinolin- | | 7.95 (d, J = 5.8 Hz, 1H), 7.44 |
| | 1-amine | | (d, J = 8.7 Hz, 1H), 7.31-7.26 |
| | | | (m, 2H), 7.17 (d, J = 5.7 Hz, |
| | | | 1H), 4.34-4.27 (m, 1H), 4.14- |
| | | | 4.02 (m, 2H), 2.17-2.07 (m, |
| | | | 1H), 1.89-1.71 (m, 3H), 1.22 |
| | | | (s, 3H), 1.20 (s, 3H). |
| 406 | N-(6-chloropyridin-3-yl)- | 370.2 (M + 1), | ¹H NMR (500 MHZ, DMSO- |
| | 6-((2-methyltetra- | 372.2 (M + 1) | d₆) δ 9.39 (s, 1H), 8.89 (d, |
| | hydrofuran-2- | | J = 2.8 Hz, 1H), 8.48-8.39 (m, |
| | yl)methoxy)isoquinolin- | | 2H), 7.96 (d, J = 5.8 Hz, 1H), |
| | 1-amine | | 7.45 (d, J = 8.7 Hz, 1H), |
| | | | 7.33-7.28 (m, 2H), 7.18 (d, |
| | | | J = 5.7 Hz, 1H), 4.03-3.94 (m, |
| | | | 2H), 3.85-3.74 (m, 2H), 2.06- |
| | | | 1.90 (m, 3H), 1.75-1.68 (m, |
| | | | 1H), 1.30 (s, 3H). |
| 407 | N-(6-chloropyridin-3-yl)- | 376.2 (M + 1), | ¹H NMR (500 MHZ, DMSO- |
| | 6-((3,3-difluorocyclo- | 378.0 (M + 1) | d₆) δ 9.38 (s, 1H), 8.88 (d, |
| | butyl)methoxy)isoquinolin- | | J = 2.8 Hz, 1H), 8.47-8.40 (m, |
| | 1-amine | | 2H), 7.96 (d, J = 5.8 Hz, 1H), |
| | | | 7.44 (d, J = 8.7 Hz, 1H), |
| | | | 7.33-7.26 (m, 2H), 7.18 (d, |
| | | | J = 5.8 Hz, 1H), 4.21 (d, J = |
| | | | 6.5 Hz, 2H), 2.82-2.71 (m, |
| | | | 2H), 2.70-2.61 (m, 1H), 2.58- |
| | | | 2.52 (m, 2H). |
| 408 | N-(6-chloropyridin-3-yl)- | 344.2 (M + 1), | ¹H NMR (500 MHZ, DMSO- |
| | 6-((1r,3r)-3-fluorocyclo- | 346.2 (M + 1) | d₆) δ 9.38 (s, 1H), 8.87 (d, |
| | butoxy)isoquinolin- | | J = 2.8 Hz, 1H), 8.47-8.40 (m, |
| | 1-amine | | 2H), 7.96 (d, J = 5.8 Hz, 1H), |
| | | | 7.44 (d, J = 8.7 Hz, 1H), 7.25 |
| | | | (dd, J = 9.2, 2.6 Hz, 1H), |
| | | | 7.18 (d, J = 5.7 Hz, 1H), 7.14 |
| | | | (d, J = 2.6 Hz, 1H), 5.04-4.85 |
| | | | (m, 1H), 4.57-4.48 (m, 1H), |
| | | | 3.19-3.09 (m, 2H), 2.37-2.23 |
| | | | (m, 2H). |
| 409 | N-(6-chloropyridin-3-yl)- | 386.2 (M + 1), | ¹H NMR (300 MHZ, DMSO- |
| | 6-(2,2,3,3-tetrafluoro- | 388.2 (M + 1) | d₆) δ 9.44 (s, 1H), 8.92-8.85 |
| | propoxy)isoquinolin-1- | | (m, 1H), 8.51-8.40 (m, 2H), |
| | amine | | 8.00 (d, J = 5.8 Hz, 1H), |
| | | | 7.50-7.34 (m, 3H), 7.19 (d, |
| | | | J = 5.7 Hz, 1H), 6.74 (tt, J = |
| | | | 51.9, 5.5 Hz, 1H), 4.85-4.72 |
| | | | (m, 2H). |
| 410 | N-(6-chloropyridin-3-yl)- | 316.0 (M + 1), | ¹H NMR (300 MHZ, DMSO- |
| | 4,6-dimethoxyisoquinolin- | 318.0 (M + 1) | d₆) 89.18 (s, 1H), 8.84 (dd, |
| | 1-amine | | J = 2.9, 0.5 Hz, 1H), 8.40 (d, |
| | | | J = 9.1 Hz, 1H), 8.34 (dd, J = |
| | | | 8.8, 2.9 Hz, 1H), 7.68 (s, |
| | | | 1H), 7.43-7.30 (m, 3H), 3.96 |
| | | | (s, 3H), 3.92 (s, 3H). |
| 411 | N-(6-chloropyridin-3-yl)- | 326.2 (M + 1), | ¹H NMR (300 MHZ, DMSO- |
| | 6-cyclobutoxyisoquinolin- | 328.2 (M + 1) | d₆) δ 9.36 (s, 1H), 8.87 (dd, |
| | 1-amine | | J = 2.9, 0.6 Hz, 1H), 8.46-8.38 |
| | | | (m, 2H), 7.95 (d, J = 5.8 Hz, |
| | | | 1H), 7.44 (dd, J = 8.7, 0.4 |
| | | | Hz, 1H), 7.24-7.10 (m, 3H), |
| | | | 4.91-4.81 (m, 1H), 2.59-2.52 |
| | | | (m, 2H), 2.17-2.02 (m, 2H), |
| | | | 1.90-1.62 (m, 2H). |
| 412 | N-(6-chloropyridin-3-yl)- | 362.0 (M + 1), | ¹H NMR (300 MHZ, DMSO- |
| | 6-(3,3-difluorocyclo- | 364.0 (M + 1) | d₆) δ 9.40 (s, 1H), 8.87 (dd, |
| | butoxy)isoquinolin- | | J = 2.8, 0.5 Hz, 1H), 8.52-8.37 |
| | 1-amine | | (m, 2H), 7.98 (d, J = 5.8 Hz, |
| | | | 1H), 7.46-7.43 (m, 1H), 7.32- |
| | | | 7.15 (m, 3H), 5.01-4.87 (m, |
| | | | 1H), 3.41-3.25 (m, 2H), 2.85- |
| | | | 2.68 (m, 2H). |
| 413 | N-(6-chloropyridin-3-yl)- | 344.0 (M + 1), | ¹H NMR (300 MHz, DMSO- |
| | 6-(((1S,2R)-2-fluorocyclo- | 346.2 (M + 1) | d₆) 89.37 (s, 1H), 8.88 (dd, |
| | propyl)methoxy)isoquinolin- | | J = 2.8, 0.5 Hz, 1H), 8.48-8.36 |
| | 1-amine | | (m, 2H), 7.96 (d, J = 5.8 Hz, |
| | | | 1H), 7.48-7.40 (m, 1H), 7.34- |
| | | | 7.22 (m, 2H), 7.16 (d, J = 5.8 |
| | | | Hz, 1H), 4.98-4.66 (m, 1H), |
| | | | 4.09-3.90 (m, 2H), 1.93-1.70 |
| | | | (m, 1H), 1.29-1.09 (m, 1H), |
| | | | 0.89-0.71 (m, 1H). |
| 414 | N-(6-chloropyridin-3-yl)- | 344.2 (M + 1), | ¹H NMR (300 MHZ, DMSO- |
| | 6-(((1S,2S)-2-fluorocyclo- | 346.0 (M + 1) | d₆) δ 9.38 (s, 1H), 8.88 (dd, |
| | propyl)methoxy)isoquinolin- | | J = 2.9, 0.5 Hz, 1H), 8.49-8.39 |
| | 1-amine | | (m, 2H), 7.96 (d, J = 5.8 Hz, |
| | | | 1H), 7.45 (dd, J = 8.8, 0.4 |
| | | | Hz, 1H), 7.36-7.26 (m, 2H), |
| | | | 7.18 (d, J = 5.7 Hz, 1H), |
| | | | 5.12-4.78 (m, 1H), 4.48-4.32 |
| | | | (m, 1H), 4.18-4.03 (m, 1H), |
| | | | 1.58-1.38 (m, 1H), 1.09-0.78 |
| | | | (m, 2H). |
| 415 | N-(6-chloropyridin-3-yl)- | 356.2(M + 1), | ¹H NMR (500 MHZ, DMSO- |
| | 6-((tetrahydrofuran-3- | 358.0 (M + 1) | d₆) δ 9.37 (s, 1H), 8.89-8.86 |
| | yl)methoxy)isoquinolin- | | (m, 1H), 8.45-8.39 (m, 2H), |
| | 1-amine | | 7.96 (d, J = 5.8 Hz, 1H), 7.44 |
| | | | (d, J = 8.7 Hz, 1H), 7.32-7.25 |
| | | | (m, 2H), 7.17 (d, J = 5.8 Hz, |
| | | | 1H), 4.12-4.02 (m, 2H), 3.85- |
| | | | 3.77 (m, 2H), 3.71-3.66 (m, |
| | | | 1H), 3.58 (dd, J = 8.7, 5.5 |
| | | | Hz, 1H), 2.77-2.68 (m, 1H), |
| | | | 2.10-2.03 (m, 1H), 1.75-1.65 |
| | | | (m, 1H). |
| 416 | (R)-N-(6-chloropyridin- | 384.2(M + 1), | ¹H NMR (500 MHZ, DMSO- |
| | 3-yl)-6-((5,5- | 386.0 (M + 1) | d₆) δ 9.37 (s, 1H), 8.88 (d, |
| | dimethyltetrahydrofuran- | | J = 2.6 Hz, 1H), 8.46-8.39 (m, |
| | 2-yl)methoxy)isoquinolin- | | 2H), 7.95 (d, J = 5.8 Hz, 1H), |
| | 1-amine | | 7.46-7.41 (m, 1H), 7.30-7.24 |
| | | | (m, 2H), 7.17 (d, J = 5.7 Hz, |
| | | | 1H), 4.34-4.27 (m, 1H), 4.13- |
| | | | 4.08 (m, 1H), 4.07-4.00 (m, |
| | | | 1H), 2.16-2.07 (m, 1H), 1.88- |
| | | | 1.71 (m, 3H), 1.21 (d, J = |
| | | | 11.7 Hz, 6H). |
| 417 | N-(6-chloropyridin-3-yl)- | 336.0(M + 1), | ¹H NMR (500 MHZ, DMSO- |
| | 6-(2,2-difluoro- | 338.0 (M + 1) | d₆) δ 9.41 (s, 1H), 8.88 (d, |
| | ethoxy)isoquinolin- | | J = 2.8 Hz, 1H), 8.51-8.40 (m, |
| | 1-amine | | 2H), 7.99 (d, J = 5.8 Hz, 1H), |
| | | | 7.45 (d, J = 8.7 Hz, 1H), |
| | | | 7.42-7.33 (m, 2H), 7.19 (d, |
| | | | J = 5.7 Hz, 1H), 6.60-6.37 (m, |
| | | | 1H), 4.53-4.46 (m, 2H). |

### Example 418

### Synthesis of 1-(6-chloropyridin-3-yl)amino)-N-(2-methoxyethyl)isoquinoline-6-carboxamide

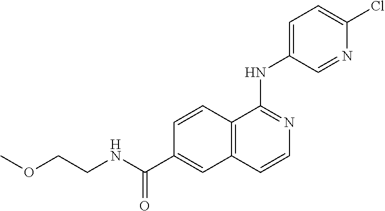 [SMILES: COCCNC(=O)c1ccc2c(Nc3ccc(Cl)nc3)nccc2c1]

### Step 1. Preparation of 1-((6-chloropyridin-3-yl)amino)isoquinoline-6-carboxylic acid

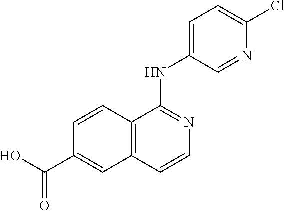 [SMILES: O=C(O)c1ccc2c(Nc3ccc(Cl)nc3)nccc2c1]

To a solution of methyl 1-((6-chloropyridin-3-yl)amino)isoquinoline-6-carboxylate (0.227 g, 0.724 mmol) in tetrahydrofuran (15 mL) and methanol (7 mL) was added a solution of lithium hydroxide monohydrate (0.046 g, 1.09 mmol) in water (7 mL). The reaction mixture was stirred at ambient temperature for 1 h. To it was then added another portion of lithium hydroxide monohydrate (0.076 g, 1.81 mmol) in water (2 mL). The mixture was stirred at ambient temperature for 5 h and then concentrated in vacuo. The residue was diluted with water (10 mL) and washed with ethyl acetate (3×10 mL). The aqueous layer was acidified with a 1 M hydrochloric acid. The formed precipitate was filtered off and washed with water (2×5 mL) and heptane (2×5 mL) to afford the title compound as a yellowish solid (0.080 g, 37% yield). Another portion of the title compound was obtained from the combined ethyl acetate phase. The combined phase was washed with brine (1×10 mL), dried over anhydrous magnesium sulfate, and filtered. The filtrate was concentrated in vacuo to afford the title compound as a pale yellow solid (0.060 g, 28% yield): ¹H NMR (300 MHz, DMSO-d₆) δ 13.39 (s, 1H), 9.62 (s, 1H), 8.93-8.88 (m, 1H), 8.62 (d, J=9.0 Hz, 1H), 8.50 (d, J=1.6 Hz, 1H), 8.43 (dd, J=8.8, 2.8 Hz, 1H), 8.14-8.06 (m, 2H), 7.49-7.44 (m, 2H); MS (ES−) m/z 298.6 (M−1), 300.6 (M−1).

### Step 2. Preparation of ((6-chloropyridin-3-yl)amino)-N-(2-methoxyethyl)isoquinoline-6-carboxamide

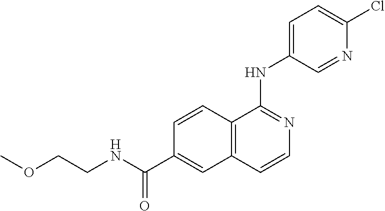 [SMILES: COCCNC(=O)c1ccc2c(Nc3ccc(Cl)nc3)nccc2c1]

To a solution of 1-((6-chloropyridin-3-yl)amino)isoquinoline-6-carboxylic acid (0.080 g, 0.267 mmol) in N,N-dimethylformamide (8 mL) was added 2-methoxyethylamine (0.040 g, 0.534 mmol), 1-[bis(dimethylamino)methylene]-1H-1,2,3-triazolo[4,5-b]pyridinium 3-oxide hexafluorophosphate (0.213 g, 0.561 mmol), and N,N-diisopropylethylamine (0.23 mL, 1.34 mmol). The reaction mixture was stirred at ambient temperature for 2.5 h and was then diluted with water (30 mL) and extracted with ethyl acetate (3×10 mL). The combined organic layers were washed with brine (1×10 mL), dried over anhydrous magnesium sulfate, and filtered. The filtrate was concentrated in vacuo. The obtained residue was triturated in methanol and the solid was filtered off to afford the title compound as a colorless solid (0.065 g, 68% yield): ¹H NMR (300 MHz, DMSO-d₆) δ 9.57 (s, 1H), 8.91 (dd, J=2.8, 0.5 Hz, 1H), 8.87-8.80 (m, 1H), 8.59 (d, J=8.9 Hz, 1H), 8.45 (dd, J=8.8, 2.9 Hz, 1H), 8.35 (d, J=1.7 Hz, 1H), 8.12-8.03 (m, 2H), 7.51-7.44 (m, 1H), 7.36 (d, J=5.7 Hz, 1H), 3.56-3.45 (m, 4H), 3.29 (s, 3H); MS (ES+) m/z 357.0 (M+1), 359.2 (M+1).

### Example 419

### Synthesis of 1-((6-chloropyridin-3-yl)amino)-N-(cyclopropylmethyl)isoquinoline-6-carboxamide

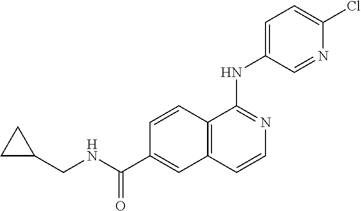 [SMILES: O=C(NCC1CC1)c1ccc2c(Nc3ccc(Cl)nc3)nccc2c1]

Following the procedure as described for EXAMPLE 418, Step 2, and making variations as required to replace 2-methoxyethylamine with cyclopropylmethanamine, the title compound was obtained as a pale yellow solid (0.052 g, 55% yield): ¹H NMR (300 MHz, DMSO-d₆) δ 9.58 (s, 1H), 8.95-8.82 (m, 2H), 8.60 (d, J=8.9 Hz, 1H), 8.45 (dd, J=8.8, 2.9 Hz, 1H), 8.35 (d, J=1.6 Hz, 1H), 8.12-8.02 (m, 2H), 7.51-7.45 (m, 1H), 7.37 (d, J=5.7 Hz, 1H), 3.21 (t, J=6.2 Hz, 2H), 1.15-1.00 (m, 1H), 0.52-0.41 (m, 2H), 031-0.22 (m, 2H); MS (ES+) m/z 353.2 (M+1), 355.2 (M+1).

### Example 420

### Synthesis of N-(2-chloropyrimidin-5-yl)-6-(5,5-dimethyltetrahydrofuran-2-yl)methoxy)isoquinolin-1-amine

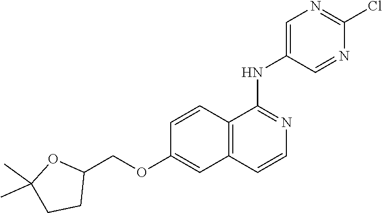 [SMILES: CC1(C)CCC(COc2ccc3c(Nc4cnc(Cl)nc4)nccc3c2)O1]

### Step 1. Preparation of 1-chloro-6-((5,5-dimethyltetrahydrofuran-2-yl)methoxy)isoquinoline

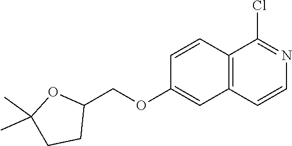 [SMILES: CC1(C)CCC(COc2ccc3c(Cl)nccc3c2)O1]

Following the procedure as described for EXAMPLE 66, Step 1, and making variations as required to replace 2,2-difluoroethanol with (5,5-dimethyloxolan-2-yl)methanol, the title compound was obtained as a pale orange oil that solidified on standing (0.60 g, 74% yield): MS (ES+) m/z 292.2 (M+1), 294.0 (M+1).

### Step 2. Preparation of N-(2-chloropyrimidin-5-yl)-6-(5-dimethyltetrahydrofuran-2-yl)methoxy)isoquinolin-1-amine

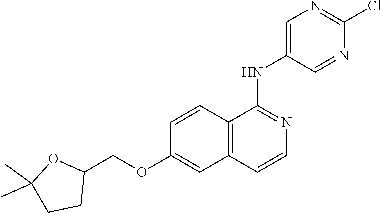 [SMILES: CC1(C)CCC(COc2ccc3c(Nc4cnc(Cl)nc4)nccc3c2)O1]

Following the same procedure as described for EXAMPLE 66, Step 2, and making variations as required to replace 1-chloro-6-(2,2-difluoroethoxy)isoquinoline with 1-chloro-6-((5,5-dimethyltetrahydrofuran-2-yl)methoxy)isoquinoline, the title compound was obtained as a pale yellow solid (0.055 g, 19% yield): ¹H NMR (500 MHz, DMSO-d₆) δ 9.56 (s, 1H), 9.29 (s, 2H), 8.43-8.39 (m, 1H), 7.99 (d, J=5.8 Hz, 1H), 7.35-7.29 (m, 2H), 7.23 (d, J=5.7 Hz, 1H), 4.35-4.27 (m, 1H), 4.14-4.09 (m, 1H), 4.07-4.01 (m, 1H), 2.16-2.10 (m, 1H), 1.90-1.71 (m, 3H), 1.21 (d, J=11.3 Hz, 6H); MS (ES+) m/z 385.2 (M+1), 387.2 (M+1).

### Example 421

### Synthesis of N-(6-chloropyridin-3-yl)-6-(pyrrolidin-1-yl)isoquinolin-1-amine

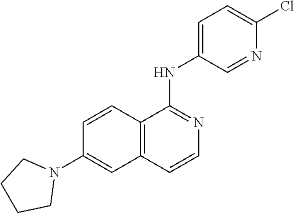 [SMILES: Clc1ccc(Nc2nccc3cc(N4CCCC4)ccc23)cn1]

### Step 1. Preparation of 2-(4-methoxybenzyl)-6-(pyrrolidin-1-yl)isoquinolin-1(2H)-one

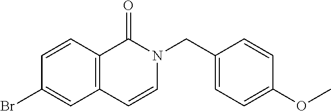 [SMILES: COc1ccc(Cn2ccc3cc(Br)ccc3c2=O)cc1]

To a solution of 6-bromoisoquinolin-1(2H)-one (7.00 g, 31.2 mmol) in N,N-dimethylacetamide (160 mL) was added sodium hydride (1.87 g, 46.9 mmol) in portions at 0° C. The reaction mixture was stirred at ambient temperature for 30 minutes, cooled to 0° C., and 4-methoxybenzyl chloride (6.3 mL, 46.9 mmol) was added dropwise to it. The reaction mixture was allowed to warm to ambient temperature and stirred for 16 h. The mixture was then cooled to 0° C. and water (300 mL) was added to it. The formed precipitate was filtered off and washed with water (2×50 mL) and heptane (2×50 mL) to afford the title compound as a colorless solid (9.02 g, 84% yield): ¹H NMR (300 MHz, CDCl₃) δ 8.33-8.27 (m, 1H), 7.68-7.63 (m, 1H), 7.57 (dd, J=8.6, 1.9 Hz, 1H), 7.30-7.25 (m, 2H), 7.10 (d, J=7.4 Hz, 1H), 6.90-6.83 (m, 2H), 6.38 (d, J=7.3 Hz, 1H), 5.13 (s, 2H), 3.78 (s, 3H); MS (ES+) 344.0 (M+1), 346.0 m/z (M+1).

### Step 2. Preparation of 2-(4-methoxybenzyl)-6-(pyrrolidin-1-yl)isoquinolin-1(2H)-one

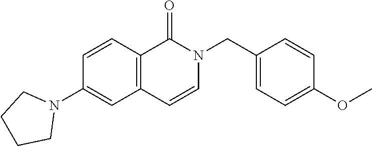 [SMILES: COc1ccc(Cn2ccc3cc(N4CCCC4)ccc3c2=O)cc1]

A mixture of 2-(4-methoxybenzyl)-6-(pyrrolidin-1-yl)isoquinolin-1(2H)-one (0.500 g, 1.45 mmol), pyrrolidine (0.15 mL, 1.80 mmol), and sodium tert-butoxide (0.202 g, 2.10 mmol) in toluene (22 mL) was purged with argon for 15 minutes. To it was added 2,2′-bis(diphenylphosphino)-1,1-binaphthalene (0.136 mg, 0.218 mmol), followed by tris(dibenzylideneacetone)dipalladium(0) (0.066 g, 0.072 mmol), and the mixture was purged with argon for 5 minutes. The reaction mixture was then stirred at 80° C. for 16 h. After cooling to ambient temperature, the mixture was diluted with water (20 mL) and extracted with dichloromethane (3×20 mL). The combined organic phase was washed with brine (20 mL), dried over anhydrous magnesium sulfate, and filtered. The filtrate was concentrated in vacuo. The residue was purified by silica gel column chromatography, eluting with a gradient of 0 to 50% of ethyl acetate in heptane, to afford the title compound as a pale yellow solid (0.24 g, 49% yield): ¹H NMR (300 MHz, CDCl₃) δ 8.27 (d, J=8.9 Hz, 1H), 7.31-7.27 (m, 1H), 7.25-7.23 (m, 1H), 6.95 (d, J=7.4 Hz, 1H), 6.87-6.81 (m, 2H), 6.75 (dd, J=9.0, 2.4 Hz, 1H), 6.38 (d, J=2.4 Hz, 1H), 6.33-6.27 (m, 1H), 5.10 (s, 2H), 3.77 (s, 3H), 3.44-3.31 (m, 4H), 2.08-2.00 (m, 4H); MS (ES+) m/z 335.2 (M+1).

### Step 3. Preparation of 6-(pyrrolidin-1-yl)isoquinolin-1(2H)-one

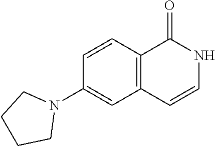 [SMILES: O=c1[nH]ccc2cc(N3CCCC3)ccc12]

A solution of 2-(4-methoxybenzyl)-6-(pyrrolidin-1-yl)isoquinolin-1(2H)-one (0.24 g, 0718 mmol) in trifluoroacetic acid (4 mL) was heated to 150° C. for 2 h in a microwave reactor. The reaction mixture was allowed to cool to ambient temperature and concentrated in vacuo. The residue was diluted with ethyl acetate (25 mL) and washed with saturated sodium bicarbonate solution (2×20 mL) and brine (20 mL). The organic phase was dried over magnesium sulfate, filtered, and the filtrate was concentrated in vacuo. The residue was purified by silica gel column chromatography, eluting with a gradient of 0 to 10% of methanol in dichloromethane, to afford the title compound as a colorless solid (0.100 g, 65% yield): ¹H NMR (300 MHz, CDCl₃) δ 10.22 (s, 1H), 8.22 (d, J=8.9 Hz, 1H), 7.07-6.97 (m, 1H), 6.81-6.73 (m, 1H), 6.46-6.42 (m, 1H), 6.37 (d, J=7.2 Hz, 1H), 3.45-3.34 (m, 4H), 2.12-1.98 (m, 4H); MS (ES+) m/z 215.2 (M+1).

### Step 4. Preparation of 1-chloro-6-(pyrrolidin-1-yl)isoquinoline

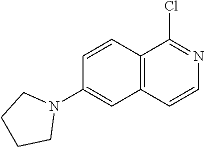 [SMILES: Clc1nccc2cc(N3CCCC3)ccc12]

A mixture of 6-(pyrrolidin-1-yl)isoquinolin-1(2H)-one (0.100 g, 0.467 mmol) and phosphorus(V) oxychloride (1 mL, 10.7 mmol) was heated to 80° C. for 4 h. The mixture was allowed to cool to ambient temperature and concentrated in vacuo. The residue was cooled to 0° C., diluted with cold water (10 mL), and the obtained mixture was poured into saturated sodium bicarbonate solution (30 mL) at 0° C. The mixture was extracted with ethyl acetate (3×25 mL). The combined organic phase was washed with brine (40 mL), dried over anhydrous magnesium sulfate, and filtered. The filtrate was concentrated in vacuo to afford the title compound as a yellowish solid (0.09 g, 83% yield): ¹H NMR (300 MHz, CDCl₃) δ 8.13-8.08 (m, 1H), 8.01 (d, J=5.8 Hz, 1H), 7.33-7.28 (m, 1H), 7.05 (dd, J=9.3, 2.4 Hz, 1H), 6.59 (d, J=2.4 Hz, 1H), 3.47-3.39 (m, 4H), 2.12-2.05 (m, 4H); MS (ES+) m/z 233.0 (M+1), 235.0 (M+1).

### Step 5. Preparation of N-(6-chloropyridin-3-yl)-6-(pyrrolidin-1-yl)isoquinolin-1-amine

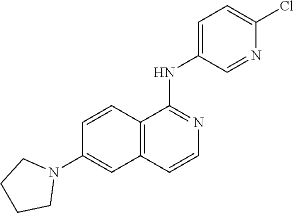 [SMILES: Clc1ccc(Nc2nccc3cc(N4CCCC4)ccc23)cn1]

A mixture of 1-chloro-6-(pyrrolidin-1-yl)isoquinoline (0.090 g, 0.3867 mmol), 5-amino-2-chloropyridine (0.050 g, 0.389 mmol), and potassium phosphate tribasic (0.25 g, 1.18 mmol) in anhydrous 1,2-dimethoxyethane (5 mL) was purged with argon for 15 minutes. To it was added 2-(dicyclohexylphosphino)-2′,4′,6′-tri-i-propyl-1,1′-biphenyl (0.018 g, 0.038 mmol) followed by tris(dibenzylideneacetone)dipalladium(0) (0.018 g, 0.020 mmol), and the mixture was purged with argon for 5 minutes. The reaction mixture was stirred at 110° C. for 16 hours. After cooling to ambient temperature, the mixture was filtered through a pad of diatomaceous earth. The pad was washed with ethyl acetate (2×20 mL) and the combined filtrate was concentrated in vacuo. The residue was purified by silica gel column chromatography, eluting with a gradient of 0 to 100% of ethyl acetate in heptane, to afford the title compound as a colorless solid (0.030 g, 24% yield): ¹H NMR (500 MHz, DMSO-d₆) δ 9.19 (s, 1H), 8.86 (d, J=2.7 Hz, 1H), 8.42 (dd, J=8.8, 2.8 Hz, 1H), 8.32-8.26 (m, 1H), 7.80 (d, J=5.8 Hz, 1H), 7.42-7.39 (m, 1H), 7.06-6.97 (m, 2H), 6.64 (d, J=2.3 Hz, 1H), 3.42-3.37 (m, 4H), 2.02-1.99 (m, 4H); MS (ES+) m/z 325.2 (M+1), 327.2 (M+1).

### Example 422

### Synthesis of N-(6-chloropyridin-3-yl)-6-(1-methyl-1H-pyrazol-5-yl)isoquinolin-1-amine

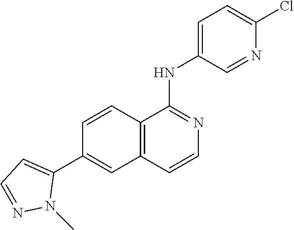 [SMILES: Cn1nccc1-c1ccc2c(Nc3ccc(Cl)nc3)nccc2c1]

To a mixture of 6-bromo-N-(6-chloropyridin-3-yl)isoquinolin-1-amine (0.050 g, 0.149 mmol), 1-methyl-5-(4,4,5,5-tetramethyl-1,3,2-dioxaborolan-2-yl)pyrazole (0.0380 g, 0.182 mmol), and potassium carbonate (0.0620 g, 0.498 mmol) in 1,4-dioxane (2 mL) and water (0.40 mL) was added [1,1′-bis(diphenylphosphino)ferrocene]dichloropalladium(II), complex with dichloromethane (0.0250 g, 0.031 mmol), and the mixture was stirred at 80° C. for 1 h. After cooling to ambient temperature, the mixture was diluted with saturated sodium bicarbonate solution (20 mL), and the mixture was extracted with ethyl acetate (3×20 mL). The combined organic phase was washed with brine (20 mL), dried over anhydrous sodium sulfate, and filtered. Concentration of the filtrate in vacuo provided a residue. Purification of the residue by silica gel column chromatography, eluting with a gradient of 0 to 20% of methanol in dichloromethane, followed by purification by preparative reverse-phase preparative HPLC, eluting with a gradient of 53 to 63% acetonitrile in water (containing 10 mM of ammonium bicarbonate), afforded the title compound as a colorless solid (0.0220 g, 44% yield): ¹H NMR (400 MHz, DMSO-d₆) δ 9.57 (s, 1H), 8.92 (s, 1H), 8.63 (d, J=8.7 Hz, 1H), 8.45 (d, J=8.9 Hz, 1H), 8.12-8.04 (m, 2H), 7.84 (d, J=8.7 Hz, 1H), 7.55 (s, 1H), 7.48 (d, J=8.8 Hz, 1H), 7.35 (d, J=5.8 Hz, 1H), 6.62 (s, 1H), 3.97 (s, 3H); MS (ES+) m/z 336.1 (M+1), 338.1 (M+1).

### Example 423

### Synthesis of N-(6-chloropyridin-3-yl)-6-(pyrimidin-5-yl)isoquinolin-1-amine

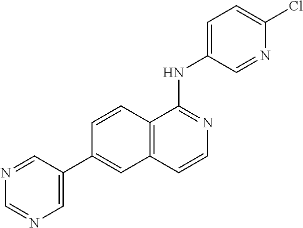 [SMILES: Clc1ccc(Nc2nccc3cc(-c4cncnc4)ccc23)cn1]

Following the procedure as described for EXAMPLE 422 and making variations as required to replace 1-methyl-5-(4,4,5,5-tetramethyl-1,3,2-dioxaborolan-2-yl)pyrazole with 5-(4,4,5,5-tetramethyl-1,3,2-dioxaborolan-2-yl)pyrimidine, the title compound was obtained as a colorless solid (0.014 g, 28% yield): ¹H NMR (400 MHz, DMSO-d₆) δ9.58 (s, 1H), 9.36 (s, 2H), 9.27 (s, 1H), 8.94 (d, J=2.8 Hz, 1H), 8.70 (d, J=8.9 Hz, 1H), 8.47 (dd, J=8.7, 2.8 Hz, 1H), 8.38 (d, J=1.8 Hz, 1H), 8.16 (dd, J=8.7, 1.9 Hz, 1H), 8.11 (d, J=5.7 Hz, 1H), 7.48 (d, J=8.7 Hz, 1H), 7.35 (d, J=5.7 Hz, 1H); MS (ES+) m/z 334.1 (M+1), 336.1 (M+1).

### Example 424

### Synthesis of N-(6-chloropyridin-3-yl)-6-(1H-pyrazol-3-yl)isoquinolin-1-amine

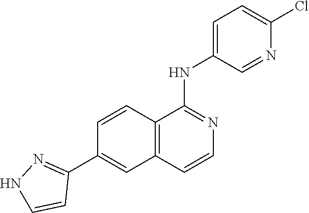 [SMILES: Clc1ccc(Nc2nccc3cc(-c4cc[nH]n4)ccc23)cn1]

Following the procedure as described for EXAMPLE 422 and making variations as required to replace 1-methyl-5-(4,4,5,5-tetramethyl-1,3,2-dioxaborolan-2-yl)pyrazole with 3-(4,4,5,5-tetramethyl-1,3,2-dioxaborolan-2-yl)-1H-pyrazole, the title compound was obtained as a colorless solid (0.0340 g, 35% yield): ¹H NMR (400 MHz, DMSO-d₆) δ 13.10 (s, 1H), 9.48 (s, 1H), 8.91 (d, J=2.9 Hz, 1H), 8.54 (d, J=8.8 Hz, 1H), 8.45 (dd, J=8.7, 2.9 Hz, 1H), 8.27 (s, 1H), 8.16 (d, J=8.7 Hz, 1H), 8.03 (d, J=5.8 Hz, 1H), 7.88 (s, 1H), 7.47 (d, J=8.8 Hz, 1H), 7.31 (d, J=5.8 Hz, 1H), 6.95 (d, J=2.1 Hz, 1H); MS (ES+) m/z 322.1 (M+1), 324.1 (M+1).

### Example 425

### Synthesis of 6-((2H-1,2,3-triazol-2-yl)methyl)-N-(6-chloropyridin-3-yl)isoquinolin-1-amine

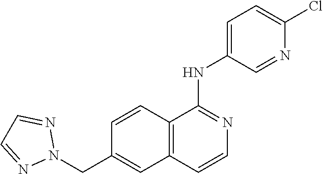 [SMILES: Clc1ccc(Nc2nccc3cc(Cn4nccn4)ccc23)cn1]

### Step 1. Preparation of (1-chloroisoquinolin-6-yl)methyl 4-methylbenzenesulfonate

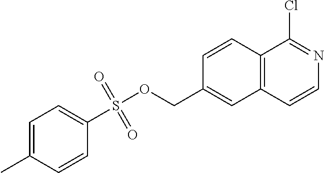 [SMILES: Cc1ccc(S(=O)(=O)OCc2ccc3c(Cl)nccc3c2)cc1]

To a solution of (1-chloroisoquinolin-6-yl)methanol (0.850 g, 4.39 mmol) and triethylamine (3.10 mL, 21.9 mmol) in tetrahydrofuran (34.0 mL) was added 4-methylbenzensulfonyl chloride (1.67 g, 8.78 mmol), and the mixture was stirred at 40° C. for 18 h. After cooling to ambient temperature, the mixture was diluted with water (300 mL) and extracted with ethyl acetate (3×300 mL). The combined organic phase was washed with brine (300 mL), dried over anhydrous sodium sulfate, and filtered. The filtrate was concentrated in vacuo and the obtained residue was purified by silica gel column chromatography, eluting with a gradient of 0 to 65% of ethyl acetate in hexanes, to afford the title compound as an oil (0.750 g, 44% yield): ¹H NMR (400 MHz, CDCl₃) δ8.27 (t, J=7.2 Hz, 2H), 7.83-7.69 (m, 3H), 7.53 (dd, J=11.9, 8.0 Hz, 2H), 7.29 (d, J=8.4 Hz, 2H), 5.23 (s, 2H), 2.40 (s, 3H); MS (ES+) m/z 348.1 (M+1), 350.1 (M+1).

### Step 2. Preparation of 6-((2H-1,2,3-triazol-2-yl)methyl)-1-chloroisoquinoline

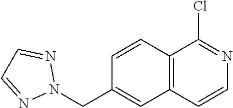 [SMILES: Clc1nccc2cc(Cn3nccn3)ccc12]

To a solution of (1-chloroisoquinolin-6-yl)methyl 4-methylbenzenesulfonate (0.150 g, 0.302 mmol) and 2H-triazole (0.045 g, 0.647 mmol) in acetone (1.5 mL) was added caesium carbonate (0.351 g, 1.08 mmol), and the mixture was stirred at ambient temperature for 2 days. The mixture was passed through a bed of celite. The filter cake was washed with dichloromethane (50 mL) and methanol (20 mL), and the combined filtrate was concentrated in vacuo. Purification of the residue by silica gel column chromatography, eluting with a gradient of 0 to 5% of methanol in dichloromethane, afforded the title compound as a colorless solid (0.023 g, 31% yield): ¹H NMR (400 MHz, CDCl₃) δ 8.31 (d, J=8.7 Hz, 1H), 8.27 (d, J=5.7 Hz, 1H), 7.71-7.69 (m, 1H), 7.68 (s, 2H), 7.59 (dd, J=8.7, 1.6 Hz, 1H), 7.56 (d, J=5.6 Hz, 1H), 5.81 (s, 2H); MS (ES+) m/z 245.8 (M+1), 247.4 (M+1).

### Step 3. Preparation of 6-((2H-1,2,3-triazol-2-yl)methyl)-N-(6-chloropyridin-3-yl)isoquinolin-1-amine

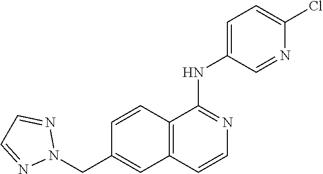 [SMILES: Clc1ccc(Nc2nccc3cc(Cn4nccn4)ccc23)cn1]

To a solution of 6-((2H-1,2,3-triazol-2-yl)methyl)-1-chloroisoquinoline (0.0580 g, 0.237 mmol), dicyclohexyl-[2-(2,4,6-triisopropylphenyl)phenyl]phosphane (0.023 g, 0.047 mmol), potassium phosphate tribasic (0.151 g, 0.711 mmol), and 6-chloropyridin-3-amine (0.034 g, 0.261 mmol) in 1,4-dioxane (1.50 mL) was added bis(dibenzylideneacetone)palladium (0.017 g, 0.0180 mmol), and the mixture was stirred at 80° C. for 12 h. After cooling to ambient temperature, the mixture was passed through a bed of celite. The solid was washed with dichloromethane (25 mL) and the combined filtrate was concentrated in vacuo. Purification of the residue by silica gel column chromatography, eluting with a gradient of 0 to 15% of methanol in dichloromethane, followed by purification by reverse-phase preparative HPLC, eluting with a gradient of 42 to 52% of acetonitrile in water (containing 10 mM of ammonium bicarbonate), afforded the title compound as a colorless solid (0.031 g, 39% yield): ¹H NMR (400 MHz, DMSO-d₆) δ 9.47 (s, 1H), 8.88 (dd, J=2.8, 0.5 Hz, 1H), 8.50 (d, J=8.8 Hz, 1H), 8.42 (dd, J=8.7, 2.9 Hz, 1H), 8.03 (d, J=5.8 Hz, 1H), 7.88 (s, 2H), 7.67 (s, 1H), 7.52 (dd, J=8.7, 1.8 Hz, 1H), 7.46 (dd, J=8.7, 0.4 Hz, 1H), 7.24 (d, J=5.6 Hz, 1H), 5.87 (s, 2H); MS (ES+) m/z 337.1 (M+1), 339.1 (M+1).

### Example 426

### Synthesis of N-(6-chloropyridin-3-yl)-6-(pyridin-2-ylmethyl)isoquinolin-1-amine

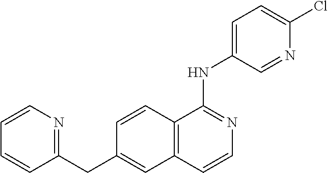 [SMILES: Clc1ccc(Nc2nccc3cc(Cc4ccccn4)ccc23)cn1]

### Step 1. Preparation of N-(6-chloropyridin-3-yl)-6-(4,4,5,5-tetramethyl-1,3,2-dioxaborolan-2-yl)isoquinolin-1-amine

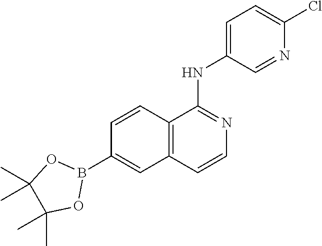 [SMILES: CC1(C)OB(c2ccc3c(Nc4ccc(Cl)nc4)nccc3c2)OC1(C)C]

To a solution of 6-bromo-N-(6-chloropyridin-3-yl)isoquinolin-1-amine (2.00 g, 5.68 mmol), 4,4,5,5-tetramethyl-2-(4,4,5,5-tetramethyl-1,3,2-dioxaborolan-2-yl)-1,3,2-dioxaborolane (1.60 g, 6.30 mmol), and potassium acetate (1.67 g, 17.00 mmol) in 1,4-dioxane (40 mL) was added [1,1′-bis(diphenylphosphino)ferrocene]dichloropalladium(II), complex with dichloromethane (0.470 g, 0.580 mmol), and the mixture was stirred at 100° C. for 18 h. After cooling to ambient temperature, the mixture was passed through a bed of celite. The filter cake was washed with methanol (50 mL) and the combined filtrate was concentrated in vacuo.

Purification of the residue by silica gel column chromatography, eluting with a gradient of 0 to 20% of ethyl acetate in hexanes, followed by purification by reverse-phase chromatography, eluting with a gradient of 5 to 100% of acetonitrile in water, afforded the title compound as a colorless solid (1.20 g, 55% yield): ¹H NMR (400 MHz, CDCl₃) δ 8.56 (dd, J=2.9, 0.6 Hz, 1H), 8.43 (dd, J=8.7, 2.9 Hz, 1H), 8.29 (s, 1H), 8.10 (d, J=5.7 Hz, 1H), 7.96 (dd, J=8.4, 1.2 Hz, 1H), 7.90 (d, J=8.5 Hz, 1H), 7.33 (dd, J=8.7, 0.6 Hz, 1H), 7.24 (d, J=5.5 Hz, 1H), 7.15 (s, 1H), 1.41 (s, 12H); MS (ES+) m/z 382.3 (M+1).

### Step 2. Preparation of N-(6-chloropyridin-3-yl)-6-(pyridin-2-ylmethyl)isoquinolin-1-amine

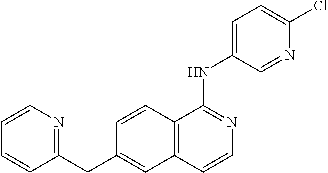 [SMILES: Clc1ccc(Nc2nccc3cc(Cc4ccccn4)ccc23)cn1]

To a solution of N-(6-chloropyridin-3-yl)-6-(4,4,5,5-tetramethyl-1,3,2-dioxaborolan-2-yl)isoquinolin-1-amine (0.250 g, 0.650 mmol), 2-(bromomethyl)pyridine hydrobromide (0.200 g, 0.790 mmol), and potassium carbonate (0.450 g, 3.28 mmol) in 1,4-dioxane (3.8 mL) and water (1.1 mL) was added [1,1′-bis(diphenylphosphino)ferrocene]dichloropalladium(II), complex with dichloromethane (0.0540 g, 0.0650 mmol), and the mixture was stirred at 100° C. for 3 h. After cooling to ambient temperature, the mixture was diluted with ethyl acetate (20 mL), and the mixture was passed through a bed of celite. The filter cake was washed with ethyl acetate (20 mL) and the combined filtrate was concentrated in vacuo. Purification of the residue by silica gel column chromatography, eluting with a gradient of 0 to 15% of methanol in dichloromethane, followed by purification by reverse-phase chromatography, eluting with a gradient of 5 to 100% of acetonitrile in water (containing 10 mM of ammonium formate), and purification of the residue by reverse-phase preparative HPLC, eluting with a gradient of 48 to 58% of acetonitrile in water (containing 10 mM of ammonium formate), afforded the title compound as a colorless solid (0.028 g, 9% yield): ¹H NMR (400 MHz, DMSO-d₆) δ 9.41 (s, 1H), 8.88 (d, J=2.9 Hz, 1H), 8.53-8.48 (m, 1H), 8.46-8.40 (m, 2H), 7.99 (d, J=5.8 Hz, 1H), 7.76-7.70 (m, 2H), 7.59 (dd, J=8.7, 1.8 Hz, 1H), 7.45 (d, J=8.7 Hz, 1H), 7.37-7.34 (m, 1H), 7.25-7.23 (m, 1H), 7.21 (d, J=5.8 Hz, 1H), 4.28 (s, 2H); MS (ES+) m/z 347.2 (M+1), 349.1 (M+1).

### Example 427

### Synthesis of 6-((3-methyl-1H-pyrazol-4-yl)methoxy)-N-(6-methylpyridin-3-yl)isoquinolin-1-amine formate

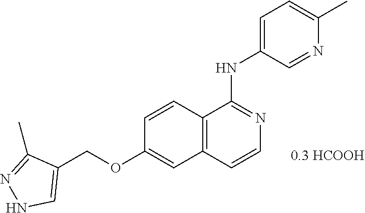 [SMILES: Cc1ccc(Nc2nccc3cc(OCc4c[nH]nc4C)ccc23)cn1]

### Step 1. Preparation of ethyl 3-methyl-1-((2-(trimethylsilyl)ethoxy)methyl)-1H-pyrazole-4-carboxylate and ethyl 5-methyl-1-((2-(trimethylsilyl)ethoxy)methyl)-1H-pyrazole-4-carboxylate

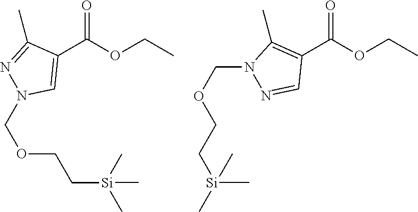

To a solution of ethyl 3-methyl-1H-pyrazole-4-carboxylate (0.615 g, 3.99 mmol) in tetrahydrofuran (25 mL) was added sodium hydride (60% dispersion in mineral oil, 0.183 g, 4.58 mmol) at ambient temperature. The reaction mixture was stirred at ambient temperature for 30 minutes and then (2-(chloromethoxy)ethyl)trimethylsilane (0.812 mL, 4.59 mmol) was added to it. The reaction mixture was stirred at ambient temperature for 16h. The reaction mixture was quenched by addition of water (20 mL) and extracted with ethyl acetate (60 mL). The organic phase was dried over anhydrous sodium sulfate, filtered, and the filtrate was concentrated in vacuo. The residue was purified by silica gel column chromatography, eluting with a gradient of 0 to 40% of ethyl acetate in heptane, to provide a mixture of the title compounds as a colorless oil (1.06 g, 94% yield): ¹H NMR (400 MHz, CDCl₃) δ7.99 (s, 1H), 7.85 (s, 1H), 5.43 (s, 2H), 5.34 (s, 2H), 4.32-4.26 (m, 4H), 3.56 (q, J=8.3 Hz, 4H), 2.61 (s, 3H), 2.47 (s, 3H), 1.35 (td, J=7.1, 4.2 Hz, 6H), 0.94-0.86 (m, 4H), −0.02 (s, 9H), −0.03 (s, 9H).

### Step 2. Preparation of (3-methyl-1-((2-(trimethylsilyl)ethoxy)methyl)-1H-pyrazol-4-yl)methanol and (5-methyl-1-((2-(trimethylsilyl)ethoxy)methyl)-1H-pyrazol-4-yl)methanol

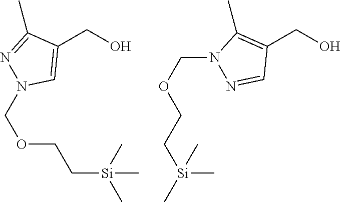

To a solution of a mixture of ethyl 3-methyl-1-((2-(trimethylsilyl)ethoxy)methyl)-1H-pyrazole-4-carboxylate and ethyl 5-methyl-1-((2-(trimethylsilyl)ethoxy)methyl)-1H-pyrazole-4-carboxylate (1.04 g, 3.66 mmol) in tetrahydrofuran (23 mL) was added dropwise a 1 M solution of lithium aluminium hydride in tetrahydrofuran (4.02 mL, 4.02 mmol) at 0° C. The reaction mixture was allowed to warm to ambient temperature and was stirred for 2h. The reaction mixture was quenched by addition of water (10 mL) and 1 N sodium hydroxide solution (20 mL). The aqueous phase was extracted with ethyl acetate (60 mL). The combined organic phase was washed with brine (50 mL), dried over anhydrous sodium sulfate, and filtered. Concentration of the filtrate in vacuo provided a mixture of the title compounds as a colorless oil (0.828 g, 93% yield): ¹H NMR (400 MHz, CDCl₃) δ 7.48 (s, 1H), 7.47 (s, 1H), 5.40 (s, 2H), 5.32 (s, 2H), 4.56 (s, 2H), 4.54 (s, 2H), 3.58-3.53 (m, 4H), 2.36 (s, 3H), 2.30 (s, 3H), 0.90 (q, J=8.3 Hz, 4H), −0.02 (s, 9H), −0.03 (s, 9H); MS (ES+) m/z 243.6 (M+1).

### Step 3. Preparation of 1-chloro-6-((3-methyl-1-((2-(trimethylsilyl)ethoxy)methyl)-1H-pyrazol-4-yl)methoxy)isoquinoline and 1-chloro-6-((5-methyl-1-((2-(trimethylsilyl)ethoxy)methyl)-1H-pyrazol-4-yl)methoxy)isoquinoline

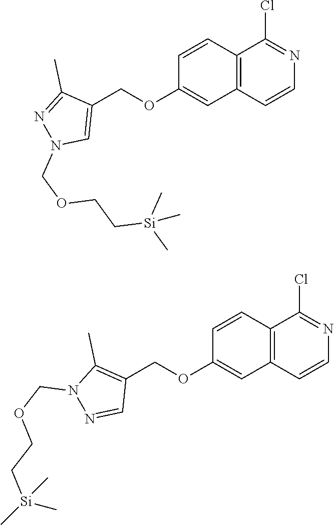 [SMILES: Cc1nn(COCC[Si](C)(C)C)cc1COc1ccc2c(Cl)nccc2c1 ; Cc1c(COc2ccc3c(Cl)nccc3c2)cnn1COCC[Si](C)(C)C]

Following the procedure as described for EXAMPLE 208, Step 1 and making variations as required to replace 3-(hydroxymethyl)oxetane-3-carbonitrile with a mixture of (3-methyl-1-((2-(trimethylsilyl)ethoxy)methyl)-1H-pyrazol-4-yl)methanol and (5-methyl-1-((2-(trimethylsilyl)ethoxy)methyl)-1H-pyrazol-4-yl)methanol, a mixture of the title compounds was obtained as a colorless oil (0.261 g, 50% yield): MS (ES+) m/z 404.6 (M+1), 406.6 (M+1).

### Step 4. 6-((3-methyl-1H-pyrazol-4-yl)methoxy)-N-(6-methylpyridin-3-yl)isoquinolin-1-amine formate

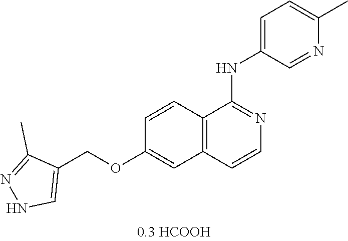 [SMILES: Cc1ccc(Nc2nccc3cc(OCc4c[nH]nc4C)ccc23)cn1]

To a solution of 1-chloro-6-((3-methyl-1-((2-(trimethylsilyl)ethoxy)methyl)-1H-pyrazol-4-yl)methoxy)isoquinoline and 1-chloro-6-((5-methyl-1-((2-(trimethylsilyl)ethoxy)methyl)-1H-pyrazol-4-yl)methoxy)isoquinoline (0.247 g, 0.611 mmol) in 1,4-dioxane (7 mL) was added 5-amino-2-methylpyridine (0.0727 g, 0.673 mmol), tris(dibenzylideneacetone)dipalladium(0) (0.0560 g, 0.0611 mmol), 2-dicyclohexylphosphino-2′,6′-dimethoxy-1,1′-biphenyl (0.0502 g, 0.122 mmol), and potassium phosphate tribasic (0.519 g, 2.45 mmol). The mixture was degassed by passing a stream of nitrogen through it for 5 minutes and then was heated to 120° C. for 2 h. After cooling to ambient temperature, the reaction mixture was filtered through a pad of celite, and the filtrate was concentrated in vacuo. The obtained residue was dissolved in dichloromethane (2 mL) and trifluoroacetic acid (1 mL) was added at ambient temperature. The reaction mixture was stirred at ambient temperature for 4 h and the volatiles were removed under reduced pressure. The residue was dissolved in ethyl acetate (30 mL) and the organic phase was washed with saturated sodium bicarbonate solution (20 mL), brine (20 mL), dried over anhydrous sodium sulfate, and concentrated in vacuo. The obtained residue was purified by reverse-phase column chromatography eluting with a gradient of 5 to 100% of acetonitrile in water containing 0.5% of formic acid, to provide the title compound as a colorless solid (0.148 g, 68% yield): ¹H NMR (400 MHz, DMSO-d₆) δ 12.67 (s, 1H), 9.14 (s, 1H), 8.84 (d, J=2.6 Hz, 1H), 8.42 (d, J=9.2 Hz, 1H), 8.19 (dd, J=8.4, 2.6 Hz, 1H), 8.14 (s, 0.3H), 7.91 (d, J=5.8 Hz, 1H), 7.66 (s, 1H), 7.37 (d, J=2.5 Hz, 1H), 7.23 (dd, J=9.2, 2.5 Hz, 1H), 7.19 (d, J=8.5 Hz, 1H), 7.11 (d, J=5.9 Hz, 1H), 5.07 (s, 2H), 2.43 (s, 3H), 2.25 (s, 3H), COOH not observed; MS (ES+) m/z 346.2 (M+1).

### Biological Example 1

### Potassium Flux Assay (In Vitro Assay)

This potassium flux assay employs the cell-permeable, potassium-sensitive dye, IPG-2 AM, to quantify potassium ion flux through potassium channels.

In general, TREX HEK 293 or HEK 293 cells were stably transfected with either an inducible or non-inducible expression vector containing the full-length cDNA coding for the desired human K_{V}7.2/K_{V}7.3 or in combination with another full-length cDNA for a second desired human K_{V}7 potassium channel. Potassium channel-expressing cell lines were induced with tetracycline (1 μg/mL), if required, and plated on 384-well poly-D-lysine (PDL)-coated plates in culture media (DMEM, containing 10% FBS and 1% L-glutamine). After overnight incubation, culture media was removed and cells were loaded with 5 μM IPG-2 AM dye for 1 hour in Assay buffer (140 mM NaCl, 20 mM RbCl, 2 mM CaCl₂, 1 mM MgCl₂, 10 mM HEPES (4-(2-hydroxyethyl)-1-piperazineethanesulfonic acid buffer), 10 mM glucose, adjusted with Tris to pH 7.4). Excess dye was removed, and cells incubated at room temperature for 20 minutes with or without test compound. A Hamamatsu FDSS μCell was used to perform a 1:1 addition of K challenge buffer (150 mM NaCl, 10 mM HEPES, 2 mM CaCl₂, 10 mM KCl, 1 mM MgCl₂, 10 mM glucose, adjusted with Tris to pH 7.4 for human K_{V}7.2/K_{V}7.3, and simultaneously read plates at excitation wavelength of 530 nm and emission wavelength of 558 nm. Non-potassium channel-mediated potassium influx was determined in the presence of DMSO, and maximal influx was determined in the presence of a known K_{V}7.x channel modulator. For each test compound, a concentration response curve was generated with 16 concentrations points, 2-fold serial dilution starting at 30 μM and an EC₅₀ value was determined.

Representative compounds of the disclosure, when tested in this model, demonstrated affinities for K_{V}7.2/K_{V}7.3 channels as set forth below in Table 2. EC₅₀ values listed are arithmetic mean values:

TABLE 2
Biological activity of representative compounds of Formula (I)

| Example No. | K_{V}7.2/K_{V}7.3 EC₅₀ (μM) |
|---|---|
| 1 | +++ |
| 2 | ++++ |
| 3 | +++ |
| 4 | +++ |
| 5 | +++ |
| 6 | +++ |
| 7 | +++ |
| 8 | +++ |
| 9 | ++++ |
| 10 | ++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | ++ |
| 22 | +++ |
| 23 | +++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | ++++ |
| 37 | ++ |
| 38 | ++ |
| 39 | +++ |
| 40 | ++++ |
| 41 | ++++ |
| 42 | ++++ |
| 43 | + |
| 44 | ++ |
| 45 | + |
| 46 | +++ |
| 47 | + |
| 48 | +++ |
| 49 | +++ |
| 50 | ++++ |
| 51 | +++ |
| 52 | ++++ |
| 53 | +++ |
| 54 | ++++ |
| 55 | ++++ |
| 56 | ++++ |
| 57 | +++ |
| 58 | + |
| 59 | ++ |
| 60 | +++ |
| 61 | +++ |
| 62 | +++ |
| 63 | +++ |
| 64 | ++++ |
| 65 | ++++ |
| 66 | ++++ |
| 67 | + |
| 68 | ++++ |
| 69 | ++ |
| 70 | ++ |
| 71 | ++ |
| 72 | ++ |
| 73 | + |
| 74 | + |
| 75 | +++ |
| 76 | + |
| 77 | ++++ |
| 78 | +++ |
| 79 | +++ |
| 80 | ++++ |
| 81 | +++ |
| 82 | +++ |
| 83 | +++ |
| 84 | ++ |
| 85 | ++++ |
| 86 | +++ |
| 87 | +++ |
| 88 | +++ |
| 89 | ++++ |
| 90 | +++ |
| 91 | ++++ |
| 92 | +++ |
| 93 | ++++ |
| 94 | ++ |
| 95 | ++++ |
| 96 | +++ |
| 97 | ++ |
| 98 | +++ |
| 99 | +++ |
| 100 | ++++ |
| 101 | +++ |
| 102 | ++++ |
| 103 | ++++ |
| 104 | ++ |
| 105 | ++ |
| 106 | +++ |
| 107 | +++ |
| 108 | +++ |
| 109 | +++ |
| 110 | +++ |
| 111 | +++ |
| 112 | +++ |
| 113 | ++++ |
| 114 | ++++ |
| 115 | + |
| 116 | +++ |
| 117 | ++++ |
| 118 | +++ |
| 119 | ++ |
| 120 | ++ |
| 121 | +++ |
| 122 | +++ |
| 123 | ++++ |
| 124 | ++++ |
| 125 | ++++ |
| 126 | ++++ |
| 127 | ++++ |
| 128 | ++ |
| 129 | +++ |
| 130 | ++ |
| 131 | + |
| 132 | ++ |
| 133 | ++ |
| 134 | ++++ |
| 135 | ++++ |
| 136 | +++ |
| 137 | ++++ |
| 138 | ++++ |
| 139 | +++ |
| 140 | ++++ |
| 141 | +++ |
| 142 | ++++ |
| 143 | ++++ |
| 144 | + |
| 145 | ++++ |
| 146 | ++++ |
| 147 | ++++ |
| 148 | ++++ |
| 149 | ++++ |
| 150 | ++++ |
| 151 | ++++ |
| 152 | ++++ |
| 153 | ++++ |
| 154 | ++++ |
| 155 | +++ |
| 156 | ++++ |
| 157 | ++++ |
| 158 | +++ |
| 159 | +++ |
| 160 | +++ |
| 161 | +++ |
| 162 | ++++ |
| 163 | ++++ |
| 16 | ++++ |
| 165 | ++++ |
| 166 | ++++ |
| 167 | +++ |
| 168 | +++ |
| 169 | +++ |
| 170 | ++++ |
| 171 | ++++ |
| 172 | ++++ |
| 173 | +++ |
| 174 | +++ |
| 175 | +++ |
| 176 | ++ |
| 177 | ++++ |
| 178 | +++ |
| 179 | +++ |
| 180 | ++ |
| 181 | ++++ |
| 182 | ++++ |
| 183 | +++ |
| 184 | +++ |
| 185 | +++ |
| 186 | +++ |
| 187 | ++++ |
| 188 | +++ |
| 189 | ++++ |
| 190 | +++ |
| 191 | +++ |
| 192 | ++++ |
| 193 | +++ |
| 194 | ++++ |
| 195 | ++++ |
| 196 | ++++ |
| 197 | ++++ |
| 198 | +++ |
| 199 | +++ |
| 200 | +++ |
| 201 | +++ |
| 202 | ++++ |
| 203 | ++++ |
| 204 | +++ |
| 205 | ++++ |
| 206 | +++ |
| 207 | ++++ |
| 208 | ++++ |
| 209 | +++ |
| 210 | ++++ |
| 211 | +++ |
| 212 | +++ |
| 213 | ++++ |
| 214 | +++ |
| 215 | ++++ |
| 216 | ++++ |
| 217 | +++ |
| 218 | ++++ |
| 219 | +++ |
| 220 | ++++ |
| 221 | +++ |
| 222 | +++ |
| 223 | ++++ |
| 224 | ++++ |
| 225 | +++ |
| 226 | +++ |
| 227 | +++ |
| 228 | +++ |
| 229 | +++ |
| 230 | ++ |
| 231 | ++++ |
| 232 | +++ |
| 233 | ++ |
| 234 | ++++ |
| 235 | ++++ |
| 236 | +++ |
| 237 | ++++ |
| 238 | ++++ |
| 239 | ++++ |
| 240 | +++ |
| 241 | ++++ |
| 242 | ++++ |
| 243 | ++ |
| 244 | +++ |
| 245 | +++ |
| 246 | +++ |
| 247 | ++ |
| 248 | +++ |
| 249 | +++ |
| 250 | ++++ |
| 251 | +++ |
| 252 | ++++ |
| 253 | ++++ |
| 254 | ++++ |
| 255 | +++ |
| 256 | +++ |
| 257 | +++ |
| 258 | +++ |
| 259 | +++ |
| 260 | ++++ |
| 261 | +++ |
| 262 | +++ |
| 263 | ++++ |
| 264 | +++ |
| 265 | ++++ |
| 266 | +++ |
| 267 | +++ |
| 268 | ++++ |
| 269 | +++ |
| 270 | +++ |
| 271 | +++ |
| 272 | +++ |
| 273 | +++ |
| 274 | ++++ |
| 275 | +++ |
| 276 | +++ |
| 277 | +++ |
| 278 | +++ |
| 279 | ++++ |
| 280 | +++ |
| 281 | ++ |
| 282 | +++ |
| 283 | +++ |
| 284 | +++ |
| 285 | +++ |
| 286 | +++ |
| 287 | +++ |
| 288 | ++++ |
| 289 | +++ |
| 290 | ++++ |
| 291 | +++ |
| 292 | ++++ |
| 293 | ++++ |
| 294 | ++++ |
| 295 | +++ |
| 296 | +++ |
| 297 | ++++ |
| 298 | +++ |
| 299 | ++++ |
| 300 | ++++ |
| 301 | ++++ |
| 302 | +++ |
| 303 | +++ |
| 304 | +++ |
| 305 | ++++ |
| 306 | ++++ |
| 307 | +++ |
| 308 | +++ |
| 309 | +++ |
| 310 | +++ |
| 311 | +++ |
| 312 | +++ |
| 313 | +++ |
| 314 | +++ |
| 315 | +++ |
| 316 | ++++ |
| 317 | +++ |
| 318 | ++++ |
| 319 | +++ |
| 320 | +++ |
| 321 | +++ |
| 322 | +++ |
| 323 | +++ |
| 324 | +++ |
| 325 | +++ |
| 326 | ++++ |
| 327 | ++++ |
| 328 | ++++ |
| 329 | +++ |
| 330 | ++++ |
| 331 | ++++ |
| 332 | +++ |
| 333 | ++++ |
| 334 | ++++ |
| 335 | +++ |
| 336 | +++ |
| 337 | +++ |
| 338 | +++ |
| 339 | ++++ |
| 340 | ++++ |
| 341 | +++ |
| 342 | +++ |
| 343 | +++ |
| 344 | +++ |
| 345 | +++ |
| 346 | ++++ |
| 347 | ++++ |
| 348 | ++++ |
| 349 | ++++ |
| 350 | +++ |
| 351 | ++++ |
| 352 | ++++ |
| 353 | ++++ |
| 354 | ++++ |
| 355 | ++++ |
| 356 | ++++ |
| 357 | ++++ |
| 358 | ++++ |
| 359 | ++++ |
| 360 | ++++ |
| 361 | ++++ |
| 362 | +++ |
| 363 | ++++ |
| 364 | ++++ |
| 365 | ++++ |
| 366 | +++ |
| 367 | ++++ |
| 368 | ++++ |
| 369 | ++++ |
| 370 | ++++ |
| 371 | ++++ |
| 372 | +++ |
| 373 | ++++ |
| 374 | ++++ |
| 375 | +++ |
| 376 | ++++ |
| 377 | ++++ |
| 378 | ++++ |
| 379 | ++++ |
| 380 | ++++ |
| 381 | ++++ |
| 382 | ++++ |
| 383 | ++++ |
| 384 | ++++ |
| 385 | ++++ |
| 386 | ++++ |
| 387 | +++ |
| 388 | ++++ |
| 389 | ++++ |
| 390 | ++++ |
| 391 | +++ |
| 392 | +++ |
| 393 | ++++ |
| 394 | +++ |
| 395 | +++ |
| 396 | ++++ |
| 397 | ++++ |
| 398 | + |
| 399 | +++ |
| 400 | ++++ |
| 401 | + |
| 402 | +++ |
| 403 | ++ |
| 404 | ++++ |
| 405 | +++ |
| 406 | ++++ |
| 407 | +++ |
| 408 | +++ |
| 409 | +++ |
| 410 | ++ |
| 411 | +++ |
| 412 | +++ |
| 413 | +++ |
| 414 | +++ |
| 415 | ++++ |
| 416 | +++ |
| 417 | +++ |
| 418 | ++ |
| 419 | ++ |
| 420 | +++ |
| 421 | +++ |
| 422 | +++ |
| 423 | ++ |
| 424 | +++ |
| 425 | +++ |
| 426 | +++ |
| 427 | ++++ |
| — | — |
| — | — |
| — | — |

For EC₅₀ values:
++++ indicates a value less than 1 μM
+++ indicates a value from 1 up to 10 μM
++ indicates a value from 10 up to 30 μM
+ indicates a value of 30 μM or more

All of the U.S. patents, U.S. patent application publications, U.S. patent applications, foreign patents, foreign patent applications and non-patent publications referred to in this specification are incorporated herein by reference in their entireties, including U.S. Provisional Application No. 63/350,180, filed Jun. 8, 2022.

Although the foregoing disclosure has been described in some detail to facilitate understanding, it will be apparent that certain changes and modifications may be practiced within the scope of the appended claims. Accordingly, the described embodiments are to be considered as illustrative and not restrictive, and the disclosure is not to be limited to the details given herein, but may be modified within the scope and equivalents of the appended claims.

1. A compound of Formula (I): 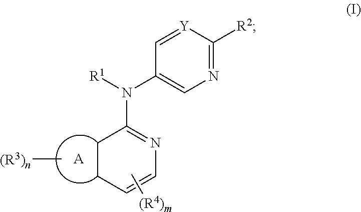
wherein:
m is 0 or 1;
n is 0, 1, 2 or 3;
Y is ═C(R⁵)— or ═N—; [symbol]
is a fused aryl or fused heteroaryl;
R¹ is hydrogen, alkyl, alkenyl, alkynyl, cycloalkyl, cycloalkylalkyl, aralkyl, heterocyclylalkyl or heteroarylalkyl;
R² is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
or R² and Y form a fused 5-membered cycloalkyl, a fused 5-membered heterocyclyl or a fused 5-membered heteroaryl;
each R³ is independently alkyl, alkenyl, alkynyl, halo, haloalkyl, haloalkenyl, cyanoalkyl, —R⁹—OR⁶, —R⁹—N(R⁶)₂, —R⁹—C(O)R⁶, —R⁹—C(O)OR⁶, —R⁹—C(O)N(R⁶)₂, —N═S(O)(R⁷)R, cycloalkyl, cycloalkylalkyl, aryl, aralkyl, heterocyclyl, heterocyclylalkyl, heteroaryl or heteroarylalkyl;
each R⁴ is independently —R⁹—OR⁶, —R⁹—N(R⁶)₂, —R⁹—C(O)R⁶, —R⁹—C(O)OR⁶, —R⁹—C(O)N(R⁶)₂, halo, alkyl, alkenyl, alkynyl, haloalkyl, haloalkenyl; cycloalkyl, cycloakylalkyl, aryl, aralkyl, heterocyclyl, heterocyclylalkyl, heteroaryl or heteroarylalkyl;
R⁵ is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aralkyl, heterocyclylalkyl or heteroarylalkyl;
each R⁶ is independently hydrogen, —R¹⁰—OR¹¹, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond, a straight or branched alkylene chain or a straight or branched alkenylene chain;
R¹⁰ is a straight or branched alkylene chain or a straight or branched alkenylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer or tautomer thereof or a mixture thereof, or a pharmaceutically acceptable salt, solvate or prodrug thereof.

2. (canceled)

3. The compound of claim 1 wherein [symbol] is a fused phenyl having the following formula (Ia): 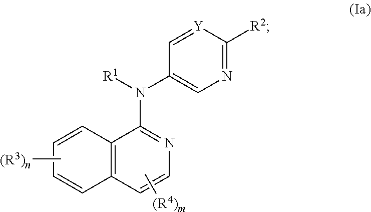
wherein m, n, Y, R¹, R², R and R⁴ are each as described above in claim 1;
as a stereoisomer, enantiomer or tautomer thereof or a mixture thereof, or a pharmaceutically acceptable salt, solvate or prodrug thereof.

4. The compound of claim 3 wherein Y is ═C(R⁵)— having the formula (Ia1): 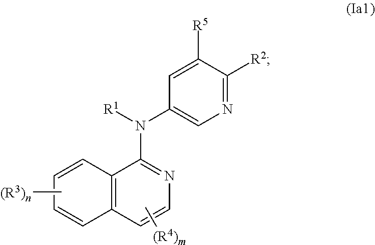
wherein m, n, R¹, R², R³, R⁴ and R⁵ are each as described above in claim 1;
as a stereoisomer, enantiomer or tautomer thereof or a mixture thereof, or a pharmaceutically acceptable salt, solvate or prodrug thereof.

5-6. (canceled)

7. The compound of claim 1 selected from the following:
N-(6-chloropyridin-3-yl)-6-(cyclopropylmethoxy)isoquinolin-1-amine;
N-(6-chloropyridin-3-yl)-6-(pyridin-2-ylmethoxy)isoquinolin-1-amine;
N-(6-chloropyridin-3-yl)-6-(2-methoxyethoxy)isoquinolin-1-amine;
N-(6-chloropyridin-3-yl)-6-isopropoxyisoquinolin-1-amine;
N-(6-chloropyridin-3-yl)-6-fluoroisoquinolin-1-amine;
N-(6-chloropyridin-3-yl)isoquinolin-1-amine;
N-(6-chloropyridin-3-yl)-6-(difluoromethoxy)isoquinolin-1-amine;
N-(6-chloropyridin-3-yl)-6-methoxyisoquinolin-1-amine;
N-(6-chloropyridin-3-yl)-6-propoxyisoquinolin-1-amine;
N-(6-chloropyridin-3-yl)-6-methylisoquinolin-1-amine;
N-(6-chloropyridin-3-yl)-6-phenoxyisoquinolin-1-amine;
N-(6-chloropyridin-3-yl)-6-((1-(trifluoromethyl)cyclopropyl)methoxy)isoquinolin-1-amine;
N-(6-chloropyridin-3-yl)-6-(cyclopropylmethoxy)-5-fluoroisoquinolin-1-amine;
N-(6-chloropyridin-3-yl)-6-(1-cyclopropylethoxy)isoquinolin-1-amine;
N¹-(6-chloropyridin-3-yl)-N⁶-(cyclopropylmethyl)isoquinoline-1,6-diamine;
methyl 1-((6-chloropyridin-3-yl)amino)isoquinoline-6-carboxylate;
((1-((6-chloropyridin-3-yl)amino)isoquinolin-6-yl)imino)dimethyl-λ⁶-sulfanone;
6-(cyclopropylmethoxy)-N-(6-methylpyridin-3-yl)isoquinolin-1-amine;
N-(6-chloropyridin-3-yl)-6-((5,5-dimethyltetrahydrofuran-2-yl)methoxy)isoquinolin-1-amine;
N-(6-chloropyridin-3-yl)-6-(pyridin-2-ylmethoxy)isoquinolin-1-amine;
N-(6-chloropyridin-3-yl)-6-(2-methoxyethoxy)isoquinolin-1-amine;
N-(6-chloropyridin-3-yl)-5-fluoroisoquinolin-1-amine;
N-(6-chloro-5-methoxypyridin-3-yl)-6-((3-methyloxetan-3-yl)methoxy)isoquinolin-1-amine;
N-(6-chloropyridin-3-yl)-6-(2-cyclopropoxyethoxy)isoquinolin-1-amine;
2-chloro-5-((6-((3-methyloxetan-3-yl)methoxy)isoquinolin-1-yl)amino)pyridin-3-ol;
N-(6-(difluoromethyl)pyridin-3-yl)-6-((1-fluorocyclopropyl)methoxy)isoquinolin-1-amine;
N-(5-chloro-6-methylpyridin-3-yl)-6-((1-fluorocyclopropyl)methoxy)isoquinolin-1-amine;
6-((1-fluorocyclopropyl)methoxy)-N-(6-(trifluoromethyl)pyridin-3-yl)isoquinolin-1-amine;
N-(5-chloropyridin-3-yl)-6-((1-fluorocyclopropyl)methoxy)isoquinolin-1-amine;
4-(((1-((6-chloropyridin-3-yl)amino)isoquinolin-6-yl)oxy)methyl)tetrahydro-2H-pyran-4-carbonitrile;
N-(6-chloropyridin-3-yl)-6-(pyrimidin-4-ylmethoxy)isoquinolin-1-amine;
N-(6-chloropyridin-3-yl)-6-((3-fluorooxetan-3-yl)methoxy)isoquinolin-1-amine;
5-(((1-((6-chloropyridin-3-yl)amino)isoquinolin-6-yl)oxy)methyl)-1-methylpyrrolidin-2-one;
N-(6-chloro-5-methoxypyridin-3-yl)-6-((1-methyl-1H-pyrazol-4-yl)methoxy)isoquinolin-1-amine;
N-(5-methoxy-6-methylpyridin-3-yl)-6-((1-methyl-1H-pyrazol-4-yl)methoxy)isoquinolin-1-amine;
N-(6-chloropyridin-3-yl)-6-((3-fluoroazetidin-3-yl)methoxy)isoquinolin-1-amine;
3-(((1-((6-chloropyridin-3-yl)amino)isoquinolin-6-yl)oxy)methyl)-1-methyl-1H-pyrazole-5-carbonitrile;
N-(6-chloropyridin-3-yl)-6-((5-methyl-1,3,4-oxadiazol-2-yl)methoxy)isoquinolin-1-amine;
N-(6-chloropyridin-3-yl)-6-((1-methyl-1H-imidazol-5-yl)methoxy)isoquinolin-1-amine;
N-(6-methoxypyridin-3-yl)-6-((3-methyloxetan-3-yl)methoxy)isoquinolin-1-amine;
1-((6-chloropyridin-3-yl)amino)isoquinolin-6-ol;
6-(2-(1-oxa-6-azaspiro[3.3]heptan-6-yl)ethoxy)-N-(6-chloropyridin-3-yl)isoquinolin-1-amine;
6-(2-(2-azaspiro[3.3]heptan-2-yl)ethoxy)-N-(6-chloropyridin-3-yl)isoquinolin-1-amine;
N-(6-chloropyridin-3-yl)-6-(2-(3-methoxyazetidin-1-yl)ethoxy)isoquinolin-1-amine;
6-(2-(2-oxa-6-azaspiro[3.3]heptan-6-yl)ethoxy)-N-(6-chloropyridin-3-yl)isoquinolin-1-amine;
6-(2-(1H-imidazol-1-yl)ethoxy)-N-(6-chloropyridin-3-yl)isoquinolin-1-amine;
2-(2-((1-((6-chloropyridin-3-yl)amino)isoquinolin-6-yl)oxy)ethyl)-1,2-dihydro-3H-pyrazol-3-one;
N-(6-chloropyridin-3-yl)-6-fluoro-N-((2-(trimethylsilyl)ethoxy)-methyl)isoquinolin-1-amine;
1-(((1-((6-chloropyridin-3-yl)amino)isoquinolin-6-yl)oxy)methyl)cyclobutane-1-carbonitrile;
3-((1-((6-chloropyridin-3-yl)amino)isoquinolin-6-yl)oxy)-2,2-dimethylpropanenitrile;
rac-(3R,4S)-4-((1-((6-chloropyridin-3-yl)amino)isoquinolin-6-yl)oxy)tetrahydrofuran-3-ol;
1-(((1-((6-chloropyridin-3-yl)amino)isoquinolin-6-yl)oxy)methyl)cyclopropane-1-carbonitrile;
6-((2-oxaspiro[3.3]heptan-6-yl)methoxy)-N-(6-chloropyridin-3-yl)isoquinolin-1-amine;
6-((1H-pyrazol-1-yl)methoxy)-N-(6-chloropyridin-3-yl)isoquinolin-1-amine;
N-(6-chloropyridin-3-yl)-6-((3,3-difluorocyclohexyl)oxy)isoquinolin-1-amine;
N-(6-chloropyridin-3-yl)-6-((6,7-dihydro-5H-pyrazolo[5,1-b][1,3]oxazin-3-yl)methoxy)isoquinolin-1-amine;
3-(((1-((6-chloropyridin-3-yl)amino)isoquinolin-6-yl)oxy)methyl)oxetane-3-carbonitrile;
N-(6-chloropyridin-3-yl)-6-((4,4-difluorotetrahydrofuran-3-yl)oxy)isoquinolin-1-amine 2,2,2-trifluoroacetate;
N-(6-chloropyridin-3-yl)-6-((4-methyl-4H-1,2,4-triazol-3-yl)methoxy)isoquinolin-1-amine;
N-(6-chloropyridin-3-yl)-6-(2-(oxetan-3-yl)ethoxy)isoquinolin-1-amine;
6-((1-benzylpiperidin-4-yl)oxy)-N-(6-chloropyridin-3-yl)isoquinolin-1-amine;
N-(6-chloropyridin-3-yl)-6-(1-(1-fluorocyclopropyl)ethoxy)isoquinolin-1-amine;
N-(6-chloropyridin-3-yl)-6-fluoro-N-((2-(trimethylsilyl)ethoxy)-methyl)isoquinolin-1-amine;
N-(6-chloropyridin-3-yl)-6-((4,4-difluorocyclohexyl)oxy)isoquinolin-1-amine;
N-(6-chloropyridin-3-yl)-6-(pyrazin-2-ylmethoxy)isoquinolin-1-amine;
rac-(1R,3S)-3-((1-((6-chloropyridin-3-yl)amino)isoquinolin-6-yl)oxy)cyclohexan-1-ol;
(1R,3R)-3-((1-((6-chloropyridin-3-yl)amino)isoquinolin-6-yl)oxy)cyclobutan-1-ol;
6-((8-benzyl-8-azabicyclo[3.2.1]octan-3-yl)oxy)-N-(6-chloropyridin-3-yl)isoquinolin-1-amine;
N-(6-chloropyridin-3-yl)-6-(2-(3-methyloxetan-3-yl)ethoxy)isoquinolin-1-amine;
N-(6-chloropyridin-3-yl)-6-(2-(1-methyl-1H-pyrazol-4-yl)ethoxy)isoquinolin-1-amine;
Cis-4-((1-((6-chloropyridin-3-yl)amino)isoquinolin-6-yl)oxy)cyclohexan-1-ol;
N-(6-chloropyridin-3-yl)-6-(2-methyl-2-morpholinopropoxy)isoquinolin-1-amine;
rac-(1S,3R)-3-((1-((6-chloropyridin-3-yl)amino)isoquinolin-6-yl)oxy)cyclopentan-1-ol;
rac-(1R,3R)-3-((1-((6-chloropyridin-3-yl)amino)isoquinolin-6-yl)oxy)cyclopentan-1-ol;
N-(6-chloropyridin-3-yl)-6-(((1s,4s)-4-methoxycyclohexyl)oxy)isoquinolin-1-amine;
N-(6-chloropyridin-3-yl)-6-(((1r,4r)-4-methoxycyclohexyl)oxy)isoquinolin-1-amine;
N-(6-chloropyridin-3-yl)-6-((1-(pyridin-3-yl)propan-2-yl)oxy)isoquinolin-1-amine;
3-(2-((1-((6-chloropyridin-3-yl)amino)isoquinolin-6-yl)oxy)ethyl)oxazolidin-2-one;
(R)-6-((1-benzylpiperidin-3-yl)oxy)-N-(6-chloropyridin-3-yl)isoquinolin-1-amine;
N-(6-chloropyridin-3-yl)-6-((1-morpholinopropan-2-yl)oxy)isoquinolin-1-amine;
ethyl 1-(((1-((6-chloropyridin-3-yl)amino)isoquinolin-6-yl)oxy)methyl)cyclopropane-1-carboxylate;
(S)-1-(3-((1-((6-chloropyridin-3-yl)amino)isoquinolin-6-yl)oxy)pyrrolidin-1-yl)ethan-1-one;
(R)-1-(3-((1-((6-chloropyridin-3-yl)amino)isoquinolin-6-yl)oxy)pyrrolidin-1-yl)ethan-1-one;
6-((2-oxaspiro[3.3]heptan-6-yl)oxy)-N-(6-chloropyridin-3-yl)isoquinolin-1-amine;
6-((1-oxaspiro[3.3]heptan-6-yl)methoxy)-N-(6-chloropyridin-3-yl)isoquinolin-1-amine;
N-(6-chloropyridin-3-yl)-6-((tetrahydro-1H-pyrrolizin-7a(5H)-yl)methoxy)isoquinolin-1-amine;
N-(6-chloropyridin-3-yl)-6-(2-(3-fluoroazetidin-1-yl)ethoxy)isoquinolin-1-amine;
N-(6-chloropyridin-3-yl)-6-((1-(oxetan-3-yl)-1H-pyrazol-4-yl)methoxy)isoquinolin-1-amine;
N-(6-chloropyridin-3-yl)-6-((1-(methylsulfonyl)piperidin-4-yl)oxy)isoquinolin-1-amine;
N-(6-chloropyridin-3-yl)-6-((1-(2-methoxyethyl)piperidin-4-yl)oxy)isoquinolin-1-amine;
6-(2-(1H-pyrazol-1-yl)ethoxy)-N-(6-chloropyridin-3-yl)isoquinolin-1-amine;
(4-((1-((6-chloropyridin-3-yl)amino)isoquinolin-6-yl)oxy)piperidin-1-yl)(cyclopropyl)methanone;
(S)-1-(3-((1-((6-chloropyridin-3-yl)amino)isoquinolin-6-yl)oxy)piperidin-1-yl)ethan-1-one;
(R)-1-(3-((1-((6-chloropyridin-3-yl)amino)isoquinolin-6-yl)oxy)piperidin-1-yl)ethan-1-one;
3-(((1-((6-chloropyridin-3-yl)amino)isoquinolin-6-yl)oxy)methyl)tetrahydrofuran-3-carbonitrile;
(R)-3-(((1-((6-chloropyridin-3-yl)amino)isoquinolin-6-yl)oxy)methyl)tetrahydrofuran-3-carbonitrile;
(S)-3-(((1-((6-chloropyridin-3-yl)amino)isoquinolin-6-yl)oxy)methyl)tetrahydrofuran-3-carbonitrile;
N-(6-chloropyridin-3-yl)-6-((3-fluoro-1-methylazetidin-3-yl)methoxy)isoquinolin-1-amine;
(R)—N-(6-chloropyridin-3-yl)-6-(1-methoxypropyl)isoquinolin-1-amine;
(S)—N-(6-chloropyridin-3-yl)-6-(1-methoxypropyl)isoquinolin-1-amine;
(R)—N-(6-chloropyridin-3-yl)-6-(1-methoxyethyl)isoquinolin-1-amine;
(S)—N-(6-chloropyridin-3-yl)-6-(1-methoxyethyl)isoquinolin-1-amine;
2-(3-(((1-((6-chloropyridin-3-yl)amino)isoquinolin-6-yl)oxy)methyl)oxetan-3-yl)acetonitrile;
(S)-6-((2-oxaspiro[3.4]octan-6-yl)oxy)-N-(6-chloropyridin-3-yl)isoquinolin-1-amine;
N-(6-chloropyridin-3-yl)-6-((2,2-dimethylbut-3-yn-1-yl)oxy)isoquinolin-1-amine;
6-([1,1′-bi(cyclopropan)]-1-ylmethoxy)-N-(6-chloropyridin-3-yl)isoquinolin-1-amine;
(R)-6-((2-oxaspiro[3.4]octan-6-yl)oxy)-N-(6-chloropyridin-3-yl)isoquinolin-1-amine;
(R)—N-(6-chloropyridin-3-yl)-6-(1-(1-fluorocyclopropyl)ethoxy)isoquinolin-1-amine;
(S)—N-(6-chloropyridin-3-yl)-6-(1-(1-fluorocyclopropyl)ethoxy)isoquinolin-1-amine;
N-(6-chloropyridin-3-yl)-6-((1-fluorocyclobutyl)methoxy)isoquinolin-1-amine;
cis-2-((1-((6-chloropyridin-3-yl)amino)isoquinolin-6-yl)oxy)cyclohexan-1-ol;
trans-2-((1-((6-chloropyridin-3-yl)amino)isoquinolin-6-yl)oxy)cyclohexan-1-ol;
(1S,3R)-3-((1-((6-chloropyridin-3-yl)amino)isoquinolin-6-yl)oxy)-1-methylcyclohexan-1-ol;
(1R,3S)-3-((1-((6-chloropyridin-3-yl)amino)isoquinolin-6-yl)oxy)-1-methylcyclohexan-1-ol;
N-(6-chloropyridin-3-yl)-6-((1-methyl-1H-pyrazol-3-yl)methoxy)isoquinolin-1-amine;
N-(6-chloropyridin-3-yl)-6-((1-ethynylcyclopropyl)methoxy)isoquinolin-1-amine;
N-(6-chloropyridin-3-yl)-6-((3-isopropyloxetan-3-yl)methoxy)isoquinolin-1-amine;
6-((1-((6-chloropyridin-3-yl)amino)isoquinolin-6-yl)oxy)spiro[3.3]heptan-2-ol;
6-((1,4-dioxan-2-yl)methoxy)-N-(6-chloropyridin-3-yl)isoquinolin-1-amine;
3-((1-((6-chloropyridin-3-yl)amino)isoquinolin-6-yl)oxy)-2,2-difluoropropan-1-ol;
N-(6-chloropyridin-3-yl)-6-((5-methylisoxazol-4-yl)methoxy)isoquinolin-1-amine;
N-(6-chloropyridin-3-yl)-6-(isoxazol-5-ylmethoxy)isoquinolin-1-amine;
N-(6-chloropyridin-3-yl)-6-((2-(pyridin-3-ylmethyl)oxazol-5-yl)methoxy)isoquinolin-1-amine;
(R)—N-(6-chloropyridin-3-yl)-6-(2-(1-methyl-1H-pyrazol-4-yl)propoxy)isoquinolin-1-amine;
(S)—N-(6-chloropyridin-3-yl)-6-(2-(1-methyl-1H-pyrazol-4-yl)propoxy)isoquinolin-1-amine;
N-(6-chloropyridin-3-yl)-6-((2,2-dimethylpent-3-yn-1-yl)oxy)isoquinolin-1-amine;
2-chloro-N³-methyl-N⁵-(6-((3-methyloxetan-3-yl)methoxy)isoquinolin-1-yl)pyridine-3,5-diamine;
2-chloro-N⁵-(6-((3-methyloxetan-3-yl)methoxy)isoquinolin-1-yl)pyridine-3,5-diamine;
N-(6-chloropyridin-3-yl)-6-((1,5-dimethyl-1H-pyrazol-4-yl)methoxy)isoquinolin-1-amine;
N-(6-chloropyridin-3-yl)-6-((1,3-dimethyl-1H-pyrazol-4-yl)methoxy)isoquinolin-1-amine;
N-(6-chloropyridin-3-yl)-6-((3-methoxy-1-methyl-1H-pyrazol-4-yl)methoxy)isoquinolin-1-amine;
N-((1s,4s)-4-((1-((6-chloropyridin-3-yl)amino)isoquinolin-6-yl)oxy)cyclohexyl)-4-methylthiazole-5-carboxamide;
6-((5-(1H-1,2,4-triazol-1-yl)pentyl)oxy)-N-(6-chloropyridin-3-yl)isoquinolin-1-amine;
N-(6-chloropyridin-3-yl)-6-(1-(1-methyl-1H-pyrazol-4-yl)ethoxy)isoquinolin-1-amine;
(R)—N-(6-chloropyridin-3-yl)-6-(1-(1-methyl-1H-pyrazol-4-yl)ethoxy)isoquinolin-1-amine;
(S)—N-(6-chloropyridin-3-yl)-6-(1-(1-methyl-1H-pyrazol-4-yl)ethoxy)isoquinolin-1-amine;
N-(6-chloropyridin-3-yl)-6-(1-(1,3-dimethyl-1H-pyrazol-4-yl)ethoxy)isoquinolin-1-amine;
(R)—N-(6-chloropyridin-3-yl)-6-(1-(1,3-dimethyl-1H-pyrazol-4-yl)ethoxy)isoquinolin-1-amine;
(S)—N-(6-chloropyridin-3-yl)-6-(1-(1,3-dimethyl-1H-pyrazol-4-yl)ethoxy)isoquinolin-1-amine;
6-(2-amino-2,3-dimethylbutoxy)-N-(6-chloropyridin-3-yl)isoquinolin-1-amine;
N-(6-chloropyridin-3-yl)-6-((1-methoxycyclopropyl)methoxy)isoquinolin-1-amine;
N-(6-chloropyridin-3-yl)-6-((1-methylcyclopropyl)methoxy)isoquinolin-1-amine;
N-(6-chloropyridin-3-yl)-6-(2-cyclopropylethoxy)isoquinolin-1-amine;
N-(6-chloropyridin-3-yl)-6-((1-fluorocyclopropyl)methoxy)isoquinolin-1-amine;
N-(6-chloropyridin-3-yl)-6-(pyrimidin-5-ylmethoxy)isoquinolin-1-amine;
N-(6-chloropyridin-3-yl)-6-(2-(pyridin-2-yl)ethoxy)isoquinolin-1-amine;
N-(6-chloropyridin-3-yl)-6-(2-(trifluoromethoxy)ethoxy)isoquinolin-1-amine;
6-(3-(1H-imidazol-1-yl)propoxy)-N-(6-chloropyridin-3-yl)isoquinolin-1-amine;
N-(6-chloropyridin-3-yl)-6-((2-methoxypyrimidin-5-yl)methoxy)isoquinolin-1-amine;
N-(5-methoxy-6-methylpyridin-3-yl)-6-((3-methyloxetan-3-yl)methoxy)isoquinolin-1-amine;
N-(6-chloropyridin-3-yl)-6-((1-ethyl-1H-pyrazol-3-yl)methoxy)isoquinolin-1-amine;
6-((1-fluorocyclopropyl)methoxy)-N-(6-methylpyridin-3-yl)isoquinolin-1-amine;
N-(6-chloropyridin-3-yl)-6-(3-(methylsulfonyl)propoxy)isoquinolin-1-amine;
N-(6-chloropyridin-3-yl)-6-(pyridin-4-ylmethoxy)isoquinolin-1-amine;
N-(6-chloropyridin-3-yl)-6-(pyrimidin-2-ylmethoxy)isoquinolin-1-amine;
N-(6-chloropyridin-3-yl)-6-((6-methylpyridin-3-yl)methoxy)isoquinolin-1-amine;
N-(6-chloropyridin-3-yl)-6-(pyridin-3-ylmethoxy)isoquinolin-1-amine;
N-(6-chloropyridin-3-yl)-6-((1-methyl-1H-pyrazol-4-yl)methoxy)isoquinolin-1-amine;
N-(6-chloropyridin-3-yl)-6-((tetrahydro-2H-pyran-4-yl)oxy)isoquinolin-1-amine;
N-(6-chloropyridin-3-yl)-6-((1-methyl-1H-imidazol-2-yl)methoxy)isoquinolin-1-amine;
N-(6-methylpyridin-3-yl)-6-(pyridin-4-ylmethoxy)isoquinolin-1-amine;
N-(6-chloropyridin-3-yl)-6-(2-morpholinoethoxy)isoquinolin-1-amine;
N-(6-chloropyridin-3-yl)-6-((1,4-dimethyl-1H-pyrazol-3-yl)methoxy)isoquinolin-1-amine;
N-(6-chloropyridin-3-yl)-6-((4,4-dimethyloxetan-2-yl)methoxy)isoquinolin-1-amine;
N-(6-chloropyridin-3-yl)-6-((3-methyloxetan-3-yl)methoxy)isoquinolin-1-amine;
N-(6-chloropyridin-3-yl)-6-(oxazol-2-ylmethoxy)isoquinolin-1-amine;
N-(6-chloropyridin-3-yl)-6-(thiazol-2-ylmethoxy)isoquinolin-1-amine;
N-(6-methylpyridin-3-yl)-6-(oxetan-3-ylmethoxy)isoquinolin-1-amine;
N-(6-chloropyridin-3-yl)-6-(isoxazol-3-ylmethoxy)isoquinolin-1-amine;
N-(6-chloropyridin-3-yl)-6-((tetrahydro-2H-pyran-3-yl)oxy)isoquinolin-1-amine;
N-(6-chloropyridin-3-yl)-6-((tetrahydrofuran-2-yl)methoxy)isoquinolin-1-amine;
N-(6-chloropyridin-3-yl)-6-((3-methoxyoxetan-3-yl)methoxy)isoquinolin-1-amine;
N-(6-chloropyridin-3-yl)-6-(oxetan-3-ylmethoxy)isoquinolin-1-amine;
(R)-3-((1-((6-chloropyridin-3-yl)amino)isoquinolin-6-yl)oxy)-1,1,1-trifluoro-2-methylpropan-2-ol;
(S)-3-((1-((6-chloropyridin-3-yl)amino)isoquinolin-6-yl)oxy)-1,1,1-trifluoro-2-methylpropan-2-ol;
6-(2-amino-3,3,3-trifluoropropoxy)-N-(6-chloropyridin-3-yl)isoquinolin-1-amine;
(R)—N-(6-chloropyridin-3-yl)-6-((4,4-difluoropyrrolidin-2-yl)methoxy)isoquinolin-1-amine;
N-(6-chloropyridin-3-yl)-N⁶-(cyclopropylmethyl)-5-fluoroisoquinoline-1,6-diamine;
1-(((1-((6-chloropyridin-3-yl)amino)isoquinolin-6-yl)oxy)methyl)-3,3-difluorocyclobutane-1-carbonitrile;
N-(6-chloropyridin-3-yl)-6-(2-(2-methoxyethoxy)ethoxy)isoquinolin-1-amine;
(S)—N-(6-chloropyridin-3-yl)-6-(1-(pyridin-4-yl)ethoxy)isoquinolin-1-amine;
(1-(((1-((6-chloropyridin-3-yl)amino)isoquinolin-6-yl)oxy)methyl)cyclopropyl)methanol;
N-(6-chloropyridin-3-yl)-6-((4-fluorotetrahydro-2H-pyran-4-yl)methoxy)isoquinolin-1-amine;
3-((1-((6-chloropyridin-3-yl)amino)isoquinolin-6-yl)oxy)-1,1,1-trifluoropropan-2-ol;
N¹-(6-chloropyridin-3-yl)-N⁶-((1-methyl-1H-pyrazol-4-yl)methyl)isoquinoline-1,6-diamine;
N¹-(6-chloropyridin-3-yl)-N⁶-((3-methyloxetan-3-yl)methyl)isoquinoline-1,6-diamine;
(E)-3-(1-((6-chloropyridin-3-yl)amino)isoquinolin-6-yl)-N,N-dimethylacrylamide;
N-(6-chloropyridin-3-yl)-7-fluoro-6-((1-fluorocyclopropyl)methoxy)isoquinolin-1-amine;
6-(((1H-pyrazol-4-yl)amino)methyl)-N-(6-chloropyridin-3-yl)isoquinolin-1-amine;
N-(6-chloropyridin-3-yl)-6-(((1-methyl-1H-pyrazol-4-yl)oxy)methyl)isoquinolin-1-amine;
N-(6-chloropyridin-3-yl)-6-((methyl(1H-pyrazol-4-yl)amino)methyl)isoquinolin-1-amine;
6-(3-(1H-pyrazol-4-yl)propoxy)-N-(6-chloropyridin-3-yl)isoquinolin-1-amine;
(R)-3-((1-((6-chloropyridin-3-yl)amino)isoquinolin-6-yl)oxy)tetrahydrothiophene 1,1-dioxide;
(S)-3-((1-((6-chloropyridin-3-yl)amino)isoquinolin-6-yl)oxy)tetrahydrothiophene 1,1-dioxide;
N-(6-chloropyridin-3-yl)-6-((tetrahydrofuran-3-yl)oxy)isoquinolin-1-amine;
trans-4-((1-((6-chloropyridin-3-yl)amino)isoquinolin-6-yl)oxy)-1-methylcyclohexan-1-ol;
cis-4-((1-((6-chloropyridin-3-yl)amino)isoquinolin-6-yl)oxy)-1-methylcyclohexan-1-ol;
N-(6-chloropyridin-3-yl)-6-(pyridin-3-yloxy)isoquinolin-1-amine;
6-((1H-pyrazol-4-yl)oxy)-N-(6-chloropyridin-3-yl)isoquinolin-1-amine;
((1-((6-chloropyridin-3-yl)amino)isoquinolin-6-yl)imino)(cyclopropyl)(methyl)-λ⁶-sulfanone;
((1-((6-chloropyridin-3-yl)amino)isoquinolin-6-yl)imino)(cyclopropylmethyl)(methyl)-λ⁶-sulfanone;
((1-((6-chloropyridin-3-yl)amino)isoquinolin-6-yl)imino)(methyl)(tetrahydro-2H-pyran-4-yl)-λ⁶-sulfanone;
((1-((6-chloropyridin-3-yl)amino)isoquinolin-6-yl)imino)(cyclopropyl)(2-methoxyethyl)-λ⁶-sulfanone;
((1-((6-chloropyridin-3-yl)amino)isoquinolin-6-yl)imino)(methyl)(oxetan-3-ylmethyl)-λ⁶-sulfanone;
(R)-((1-((6-chloropyridin-3-yl)amino)isoquinolin-6-yl)imino)(cyclopropyl)(methyl)-λ⁶-sulfanone;
(S)-((1-((6-chloropyridin-3-yl)amino)isoquinolin-6-yl)imino)(cyclopropyl)(methyl)-λ⁶-sulfanone;
((1-((6-chloropyridin-3-yl)amino)isoquinolin-6-yl)imino)(methyl)(oxetan-3-yl)-λ⁶-sulfanone;
(S)-((1-((6-chloropyridin-3-yl)amino)isoquinolin-6-yl)imino)(methyl)(oxetan-3-yl)-λ⁶-sulfanone);
(R)-((1-((6-chloropyridin-3-yl)amino)isoquinolin-6-yl)imino)(methyl)(oxetan-3-yl)-λ⁶-sulfanone);
N-(6-chloropyridin-3-yl)-6-((1-(difluoromethyl)-1H-pyrazol-4-yl)methoxy)isoquinolin-1-amine;
(R)-6-((1-benzylpyrrolidin-2-yl)methoxy)-N-(6-chloropyridin-3-yl)isoquinolin-1-amine;
N-(6-chloropyridin-3-yl)-6-((5,6-dihydro-4H-pyrrolo[1,2-b]pyrazol-3-yl)methoxy)isoquinolin-1-amine;
6-((1-benzylpyrrolidin-3-yl)oxy)-N-(6-chloropyridin-3-yl)isoquinolin-1-amine;
(R)-6-((1-benzylpyrrolidin-3-yl)methoxy)-N-(6-chloropyridin-3-yl)isoquinolin-1-amine;
(S)-6-((1-benzylpyrrolidin-3-yl)methoxy)-N-(6-chloropyridin-3-yl)isoquinolin-1-amine;
N-(6-chloropyridin-3-yl)-6-((4,5,6,7-tetrahydropyrazolo[1,5-a]pyridin-3-yl)methoxy)isoquinolin-1-amine;
N-(6-chloropyridin-3-yl)-6-((1-(methylsulfonyl)cyclopropyl)methoxy)isoquinolin-1-amine;
N-(6-chloropyridin-3-yl)-6-((1-(methylsulfonyl)cyclobutyl)methoxy)isoquinolin-1-amine;
N-(6-chloropyridin-3-yl)-6-((1-methoxycyclobutyl)methoxy)isoquinolin-1-amine;
N-(6-chloropyridin-3-yl)-6-((1-cyclopropyl-1H-pyrazol-4-yl)methoxy)isoquinolin-1-amine;
6-((2-oxaspiro[3.3]heptan-5-yl)oxy)-N-(6-chloropyridin-3-yl)isoquinolin-1-amine;
6-((5-chloro-1-methyl-1H-pyrazol-4-yl)methoxy)-N-(6-chloropyridin-3-yl)isoquinolin-1-amine;
6-((3-chloro-1-methyl-1H-pyrazol-4-yl)methoxy)-N-(6-chloropyridin-3-yl)isoquinolin-1-amine;
3-(((1-((6-chloropyridin-3-yl)amino)isoquinolin-6-yl)oxy)methyl)-3-methylthietane 1,1-dioxide;
1-(((1-((6-chloropyridin-3-yl)amino)isoquinolin-6-yl)oxy)methyl)cyclobutane-1-sulfonamide;
6-((1-benzyl-1H-pyrazol-4-yl)methoxy)-N-(6-chloropyridin-3-yl)isoquinolin-1-amine;
N-(6-chloropyridin-3-yl)-6-((1-(1-methyl-1H-pyrazol-4-yl)propan-2-yl)oxy)isoquinolin-1-amine;
(R)—N-(6-chloropyridin-3-yl)-6-((tetrahydrofuran-3-yl)oxy)isoquinolin-1-amine;
(S)—N-(6-chloropyridin-3-yl)-6-((tetrahydrofuran-3-yl)oxy)isoquinolin-1-amine;
N-(6-chloropyridin-3-yl)-6-((2,2-dimethyl-1,3-dioxolan-4-yl)methoxy)isoquinolin-1-amine;
3-(((1-((6-chloropyridin-3-yl)amino)isoquinolin-6-yl)oxy)methyl)thietane 1,1-dioxide;
N-(6-chloropyridin-3-yl)-6-(isothiazol-4-ylmethoxy)isoquinolin-1-amine;
(R)—N-(6-chloropyridin-3-yl)-6-((2-methyltetrahydrofuran-2-yl)methoxy)isoquinolin-1-amine;
(S)—N-(6-chloropyridin-3-yl)-6-((2-methyltetrahydrofuran-2-yl)methoxy)isoquinolin-1-amine;
6-(((1H-pyrazol-4-yl)oxy)methyl)-N-(6-chloropyridin-3-yl)isoquinolin-1-amine;
1-(4-((1-((6-chloropyridin-3-yl)amino)isoquinolin-6-yl)oxy)piperidin-1-yl)ethan-1-one;
N-(6-chloropyridin-3-yl)-6-((3-(methoxymethyl)oxetan-3-yl)methoxy)isoquinolin-1-amine;
N-(6-chloropyridin-3-yl)-6-((1-methyl-1H-1,2,4-triazol-3-yl)methoxy)isoquinolin-1-amine;
1-(3-(((1-((6-chloropyridin-3-yl)amino)isoquinolin-6-yl)oxy)methyl)azetidin-1-yl)ethan-1-one;
N-(6-chloropyridin-3-yl)-6-((1-(2,2,2-trifluoroethyl)azetidin-3-yl)methoxy)isoquinolin-1-amine;
N-(6-chloropyridin-3-yl)-6-((1-methyl-1H-pyrazol-4-yl)oxy)isoquinolin-1-amine;
1-(4-((1-((6-chloropyridin-3-yl)amino)isoquinolin-6-yl)oxy)piperidin-1-yl)ethan-1-one;
N-(6-chloropyridin-3-yl)-6-((1-(oxetan-3-yl)piperidin-4-yl)oxy)isoquinolin-1-amine;
N-(6-chloropyridin-3-yl)-6-((1-(pyrimidin-2-ylmethyl)piperidin-4-yl)oxy)isoquinolin-1-amine;
N-(6-chloropyridin-3-yl)-6-((1-(2,2-difluoroethyl)piperidin-4-yl)oxy)isoquinolin-1-amine;
N-(cis-4-((1-((6-chloropyridin-3-yl)amino)isoquinolin-6-yl)oxy)cyclohexyl)acetamide;
N-(trans-4-((1-((6-chloropyridin-3-yl)amino)isoquinolin-6-yl)oxy)cyclohexyl)acetamide;
1-(4-(((1-((6-chloropyridin-3-yl)amino)isoquinolin-6-yl)oxy)methyl)piperidin-1-yl)ethan-1-one;
N-(6-chloropyridin-3-yl)-6-(2-((2S,6R)-2,6-dimethylmorpholino)ethoxy)isoquinolin-1-amine;
methyl 4-(2-((1-((6-chloropyridin-3-yl)amino)isoquinolin-6-yl)oxy)ethyl)morpholine-3-carboxylate;
(S)—N-(6-chloropyridin-3-yl)-6-(2-(3-methylmorpholino)ethoxy)isoquinolin-1-amine;
(R)—N-(6-chloropyridin-3-yl)-6-(2-(3-methylmorpholino)ethoxy)isoquinolin-1-amine;
N-(6-chloropyridin-3-yl)-6-(2-(2-methylmorpholino)ethoxy)isoquinolin-1-amine;
2-methyl-5-((6-((1-methyl-1H-pyrazol-4-yl)methoxy)isoquinolin-1-yl)amino)pyridin-3-ol;
N-(6-chloropyridin-3-yl)-6-((1-(2,2,2-trifluoroethyl)azetidin-3-yl)oxy)isoquinolin-1-amine;
N-(6-chloropyridin-3-yl)-6-((1-(difluoromethyl)cyclopropyl)methoxy)isoquinolin-1-amine;
N-(6-chloropyridin-3-yl)-6-((3-(difluoromethyl)oxetan-3-yl)methoxy)isoquinolin-1-amine;
N-(6-chloropyridin-3-yl)-6-((3-ethyloxetan-3-yl)methoxy)isoquinolin-1-amine;
(3-(((1-((6-chloropyridin-3-yl)amino)isoquinolin-6-yl)oxy)methyl)oxetan-3-yl)methanol;
1-(((1-((6-chloropyridin-3-yl)amino)isoquinolin-6-yl)oxy)methyl)-2,2-dimethylcyclopropane-1-carbonitrile;
3-((1-((6-chloropyridin-3-yl)amino)isoquinolin-6-yl)oxy)adamantan-1-ol;
N-(6-chloropyridin-3-yl)-6-(spiro[2.3]hexan-1-ylmethoxy)isoquinolin-1-amine;
6-(((1-((6-chloropyridin-3-yl)amino)isoquinolin-6-yl)oxy)methyl)-2-oxaspiro[3.3]heptane-6-carbonitrile;
6-(1-(1H-pyrazol-4-yl)ethoxy)-N-(6-chloropyridin-3-yl)isoquinolin-1-amine;
6-(1-(1H-pyrazol-4-yl)ethoxy)-N-(6-chloropyridin-3-yl)isoquinolin-1-amine;
N-(6-chloropyridin-3-yl)-6-((1-(2-methoxyethyl)-1H-pyrazol-4-yl)methoxy)isoquinolin-1-amine;
6-((3-chloro-1H-pyrazol-4-yl)methoxy)-N-(6-chloropyridin-3-yl)isoquinolin-1-amine;
N-(6-chloropyridin-3-yl)-6-((3,5-dimethyl-1H-pyrazol-4-yl)methoxy)isoquinolin-1-amine;
N-(6-chloropyridin-3-yl)-6-(2-(3-methyloxetan-3-yl)ethyl)isoquinolin-1-amine;
(1S,3S)-3-((1-((6-chloropyridin-3-yl)amino)isoquinolin-6-yl)oxy)cyclohexane-1-carbonitrile;
(1R,3S)-3-((1-((6-chloropyridin-3-yl)amino)isoquinolin-6-yl)oxy)cyclohexane-1-carbonitrile;
(1R,3R)-3-((1-((6-chloropyridin-3-yl)amino)isoquinolin-6-yl)oxy)cyclohexane-1-carbonitrile;
(1S,3R)-3-((1-((6-chloropyridin-3-yl)amino)isoquinolin-6-yl)oxy)cyclohexane-1-carbonitrile;
(1R,3S)-3-((1-((6-chloropyridin-3-yl)amino)isoquinolin-6-yl)oxy)cyclohexan-1-ol;
(1S,3R)-3-((1-((6-chloropyridin-3-yl)amino)isoquinolin-6-yl)oxy)cyclohexan-1-ol;
(1R,4R)-4-((1-((6-chloropyridin-3-yl)amino)isoquinolin-6-yl)oxy)cyclohexan-1-ol;
trans-3-((1-((6-chloropyridin-3-yl)amino)isoquinolin-6-yl)oxy)cyclohexan-1-ol;
N-(6-chloropyridin-3-yl)-6-((1-(pyridin-4-ylmethoxy)cyclopropyl)methoxy)isoquinolin-1-amine;
N-(6-chloropyridin-3-yl)-6-((3-fluorotetrahydrofuran-3-yl)methoxy)isoquinolin-1-amine;
(R)—N-(6-chloropyridin-3-yl)-6-((3-fluorotetrahydrofuran-3-yl)methoxy)isoquinolin-1-amine;
(S)—N-(6-chloropyridin-3-yl)-6-((3-fluorotetrahydrofuran-3-yl)methoxy)isoquinolin-1-amine;
N-(6-chloropyridin-3-yl)-6-(isoxazol-4-ylmethoxy)isoquinolin-1-amine;
2-chloro-5-((6-((1-methyl-1H-pyrazol-4-yl)methoxy)isoquinolin-1-yl)amino)pyridin-3-ol;
N-(6-chloropyridin-3-yl)-6-((5-cyclopropyl-1H-pyrazol-4-yl)methoxy)isoquinolin-1-amine;
N-(6-chloropyridin-3-yl)-6-((1-(2,2-difluoroethyl)-1H-pyrazol-4-yl)methoxy)isoquinolin-1-amine;
N-(6-chloropyridin-3-yl)-6-((1-methyl-1H-pyrazol-4-yl)methoxy-d₂)isoquinolin-1-amine;
N-(6-chloropyridin-3-yl)-6-(2-(pyrimidin-2-yl)ethoxy)isoquinolin-1-amine;
6-((1H-pyrazol-3-yl)methoxy)-N-(6-chloropyridin-3-yl)isoquinolin-1-amine;
N-(6-chloropyridin-3-yl)-6-((5-methyl-1H-pyrazol-3-yl)methoxy)isoquinolin-1-amine;
N-(6-chloropyridin-3-yl)-6-((3-methyl-1H-pyrazol-4-yl)methoxy)isoquinolin-1-amine;
3-(((1-((6-chloropyridin-3-yl)amino)isoquinolin-6-yl)oxy)methyl)oxetan-3-ol;
N-(6-chloropyridin-3-yl)-6-((4-fluoro-1-methyl-1H-pyrazol-3-yl)methoxy)isoquinolin-1-amine;
N-(6-chloropyridin-3-yl)-3-methylisoquinolin-1-amine;
2-(4-(((1-((6-chloropyridin-3-yl)amino)isoquinolin-6-yl)oxy)methyl)-1H-pyrazol-1-yl)acetonitrile;
N-(1-((6-chloropyridin-3-yl)amino)isoquinolin-6-yl)-1-(hydroxymethyl)cyclopropane-1-carboxamide;
N-(6-chloropyridin-3-yl)-6-((2,2-difluorocyclopropyl)methoxy)isoquinolin-1-amine;
(S)—N-(6-chloropyridin-3-yl)-6-((5,5-dimethyltetrahydrofuran-2-yl)methoxy)isoquinolin-1-amine;
N-(6-chloropyridin-3-yl)-6-((2-methyltetrahydrofuran-2-yl)methoxy)isoquinolin-1-amine;
N-(6-chloropyridin-3-yl)-6-((3,3-difluorocyclobutyl)methoxy)isoquinolin-1-amine;
N-(6-chloropyridin-3-yl)-6-((1r,3r)-3-fluorocyclobutoxy)isoquinolin-1-amine;
N-(6-chloropyridin-3-yl)-6-(2,2,3,3-tetrafluoropropoxy)isoquinolin-1-amine;
N-(6-chloropyridin-3-yl)-4,6-dimethoxyisoquinolin-1-amine;
N-(6-chloropyridin-3-yl)-6-cyclobutoxyisoquinolin-1-amine;
N-(6-chloropyridin-3-yl)-6-(3,3-difluorocyclobutoxy)isoquinolin-1-amine;
N-(6-chloropyridin-3-yl)-6-(((1S,2R)-2-fluorocyclopropyl)methoxy)isoquinolin-1-amine;
N-(6-chloropyridin-3-yl)-6-(((1S,2S)-2-fluorocyclopropyl)methoxy)isoquinolin-1-amine;
N-(6-chloropyridin-3-yl)-6-((tetrahydrofuran-3-yl)methoxy)isoquinolin-1-amine;
(R)—N-(6-chloropyridin-3-yl)-6-((5,5-dimethyltetrahydrofuran-2-yl)methoxy)isoquinolin-1-amine;
N-(6-chloropyridin-3-yl)-6-(2,2-difluoroethoxy)isoquinolin-1-amine;
1-((6-chloropyridin-3-yl)amino)-N-(2-methoxyethyl)isoquinoline-6-carboxamide;
1-((6-chloropyridin-3-yl)amino)-N-(cyclopropylmethyl)isoquinoline-6-carboxamide;
N-(6-chloropyridin-3-yl)-6-(pyrrolidin-1-yl)isoquinolin-1-amine;
N-(6-chloropyridin-3-yl)-6-(1-methyl-1H-pyrazol-5-yl)isoquinolin-1-amine;
N-(6-chloropyridin-3-yl)-6-(pyrimidin-5-yl)isoquinolin-1-amine;
N-(6-chloropyridin-3-yl)-6-(1H-pyrazol-3-yl)isoquinolin-1-amine;
6-((2H-1,2,3-triazol-2-yl)methyl)-N-(6-chloropyridin-3-yl)isoquinolin-1-amine;
N-(6-chloropyridin-3-yl)-6-(pyridin-2-ylmethyl)isoquinolin-1-amine; and
6-((3-methyl-1H-pyrazol-4-yl)methoxy)-N-(6-methylpyridin-3-yl)isoquinolin-1-amine, as an individual stereoisomer, enantiomer or tautomer thereof or a mixture thereof,
or a pharmaceutically acceptable salt, solvate or prodrug thereof.

8. The compound of claim 3 wherein Y is ═N— having the following formula (Ia2): 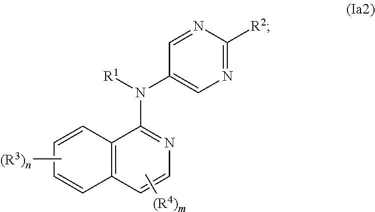
wherein m, n, R¹, R², R³ and R⁴ are each as described above in claim 3;
as a stereoisomer, enantiomer or tautomer thereof or a mixture thereof, or a pharmaceutically acceptable salt, solvate or prodrug thereof.

9-10. (canceled)

11. The compound of claim 1 selected from the following:
N-(2-chloropyrimidin-5-yl)-6-(cyclopropylmethoxy)isoquinolin-1-amine;
N-(2-chloropyrimidin-5-yl)-6-fluoroisoquinolin-1-amine;
6-chloro-N-(2-chloropyrimidin-5-yl)isoquinolin-1-amine;
6-(cyclopropylmethoxy)-N-(2-methylpyrimidin-5-yl)isoquinolin-1-amine;
N-(2-methylpyrimidin-5-yl)-6-((tetrahydrofuran-3-yl)methoxy)isoquinolin-1-amine;
6-(2,2-difluoroethoxy)-N-(2-methylpyrimidin-5-yl)isoquinolin-1-amine;
6-((5,5-dimethyltetrahydrofuran-2-yl)methoxy)-N-(2-methylpyrimidin-5-yl)isoquinolin-1-amine;
6-(2-methoxyethoxy)-N-(2-methylpyrimidin-5-yl)isoquinolin-1-amine;
6-(2-cyclopropoxyethoxy)-N-(2-methylpyrimidin-5-yl)isoquinolin-1-amine;
3-(((1-((2-chloropyrimidin-5-yl)amino)isoquinolin-6-yl)oxy)methyl)bicyclo[1.1.1]pentane-1-carbonitrile;
N-(2-chloropyrimidin-5-yl)-6-((3-isopropyloxetan-3-yl)methoxy)isoquinolin-1-amine;
N-(2-ethylpyrimidin-5-yl)-6-((1-fluorocyclopropyl)methoxy)isoquinolin-1-amine;
6-((1-fluorocyclopropyl)methoxy)-N-(2-methoxypyrimidin-5-yl)isoquinolin-1-amine;
6-((1-fluorocyclopropyl)methoxy)-N-(pyrimidin-5-yl)isoquinolin-1-amine;
N-(2-chloropyrimidin-5-yl)-6-((3-fluorooxetan-3-yl)methoxy)isoquinolin-1-amine;
6-((3-fluorooxetan-3-yl)methoxy)-N-(2-methylpyrimidin-5-yl)isoquinolin-1-amine;
6-((3-fluoroazetidin-3-yl)methoxy)-N-(2-methylpyrimidin-5-yl)isoquinolin-1-amine;
1-((1-((2-chloropyrimidin-5-yl)amino)isoquinolin-6-yl)oxy)cyclopropane-1-carbonitrile;
1-((1-((2-chloropyrimidin-5-yl)amino)isoquinolin-6-yl)oxy)cyclopropane-1-carboxamide;
(1s,3s)-3-(((1-((2-chloropyrimidin-5-yl)amino)isoquinolin-6-yl)oxy)methyl)-3-fluorocyclobutane-1-carbonitrile;
N-(2-chloropyrimidin-5-yl)-6-((2-fluorocyclopropyl)methoxy)isoquinolin-1-amine;
1-(((1-((2-methylpyrimidin-5-yl)amino)isoquinolin-6-yl)oxy)methyl)cyclopropane-1-carbonitrile;
1-(((1-((2-methylpyrimidin-5-yl)amino)isoquinolin-6-yl)oxy)methyl)cyclopropane-1-carbonitrile;
(5-((6-((1-fluorocyclopropyl)methoxy)isoquinolin-1-yl)amino)pyrimidin-2-yl)methanol;
N-(2-chloropyrimidin-5-yl)-6-((1-fluorocyclopropyl)methoxy)-N-methylisoquinolin-1-amine;
rac-(1R,3S)-3-((1-((2-chloropyrimidin-5-yl)amino)isoquinolin-6-yl)oxy)-1-(trifluoromethyl)cyclohexan-1-ol;
3-((1-((2-chloropyrimidin-5-yl)amino)isoquinolin-6-yl)oxy)-2-cyclopropyl-2-fluoropropanenitrile;
2-(1-(((1-((2-chloropyrimidin-5-yl)amino)isoquinolin-6-yl)oxy)methyl)cyclopropyl)acetonitrile;
6-(1-(1-methyl-1H-pyrazol-4-yl)ethoxy)-N-(2-methylpyrimidin-5-yl)isoquinolin-1-amine;
6-((1-methoxycyclopropyl)methoxy)-N-(2-methylpyrimidin-5-yl)isoquinolin-1-amine;
6-((1-fluorocyclopropyl)methoxy)-N-(2-methylpyrimidin-5-yl)isoquinolin-1-amine;
N-(2-methylpyrimidin-5-yl)-6-(pyridin-4-ylmethoxy)isoquinolin-1-amine;
N-(2-chloropyrimidin-5-yl)-6-(pyridin-4-ylmethoxy)isoquinolin-1-amine;
N-(2-chloropyrimidin-5-yl)-6-((1-methyl-1H-pyrazol-3-yl)methoxy)isoquinolin-1-amine;
6-((1-methyl-1H-pyrazol-4-yl)methoxy)-N-(2-methylpyrimidin-5-yl)isoquinolin-1-amine;
6-((1-methyl-1H-pyrazol-3-yl)methoxy)-N-(2-methylpyrimidin-5-yl)isoquinolin-1-amine;
1-(((1-((2-chloropyrimidin-5-yl)amino)isoquinolin-6-yl)oxy)methyl)cyclopropane-1-carbonitrile;
3-(((1-((2-chloropyrimidin-5-yl)amino)isoquinolin-6-yl)oxy)methyl)oxetane-3-carbonitrile;
N-(2-chloropyrimidin-5-yl)-6-(oxetan-3-ylmethoxy)isoquinolin-1-amine;
6-((3-methyloxetan-3-yl)methoxy)-N-(2-methylpyrimidin-5-yl)isoquinolin-1-amine;
6-((3-methoxyoxetan-3-yl)methoxy)-N-(2-methylpyrimidin-5-yl)isoquinolin-1-amine;
N-(2-methylpyrimidin-5-yl)-6-(oxetan-3-ylmethoxy)isoquinolin-1-amine;
6-(isoxazol-3-ylmethoxy)-N-(2-methylpyrimidin-5-yl)isoquinolin-1-amine;
3-(((1-((2-methylpyrimidin-5-yl)amino)isoquinolin-6-yl)oxy)methyl)oxetane-3-carbonitrile;
6-(cyclopropylmethoxy)-5-fluoro-N-(2-methylpyrimidin-5-yl)isoquinolin-1-amine;
3,3-difluoro-1-(((1-((2-methylpyrimidin-5-yl)amino)isoquinolin-6-yl)oxy)methyl)cyclobutane-1-carbonitrile;
6-(isoxazol-4-ylmethoxy)-N-(2-methylpyrimidin-5-yl)isoquinolin-1-amine;
N-(2-chloropyrimidin-5-yl)-6-(isoxazol-4-ylmethoxy)isoquinolin-1-amine;
1-((2-chloropyrimidin-5-yl)amino)isoquinolin-6-ol;
cyclopropyl(methyl)((1-((2-methylpyrimidin-5-yl)amino)isoquinolin-6-yl)imino)-λ6-sulfanone;
N-(2-methylpyrimidin-5-yl)-6-(pyrimidin-5-ylmethoxy)isoquinolin-1-amine;
(1R,3S)-3-((1-((2-methylpyrimidin-5-yl)amino)isoquinolin-6-yl)oxy)cyclohexan-1-ol;
(1S,3R)-3-((1-((2-methylpyrimidin-5-yl)amino)isoquinolin-6-yl)oxy)cyclohexan-1-ol;
cis-3-((1-((2-chloropyrimidin-5-yl)amino)isoquinolin-6-yl)oxy)cyclohexan-1-ol;
1-(((1-((2-chloropyrimidin-5-yl)amino)isoquinolin-6-yl)oxy)methyl)spiro[2.2]pentane-1-carbonitrile;
1-(((1-((2-chloropyrimidin-5-yl)amino)isoquinolin-6-yl)oxy)methyl)-2,2-dimethylcyclopropane-1-carbonitrile;
N-(2-chloropyrimidin-5-yl)-6-((1,5-dimethyl-1H-pyrazol-4-yl)methoxy)isoquinolin-1-amine;
6-((1,5-dimethyl-1H-pyrazol-4-yl)methoxy)-N-(2-methylpyrimidin-5-yl)isoquinolin-1-amine;
N-(2-chloropyrimidin-5-yl)-6-((3-(1,1-difluoroethyl)oxetan-3-yl)methoxy)isoquinolin-1-amine;
tert-butyl ((1S,3R)-3-((1-((2-chloropyrimidin-5-yl)amino)isoquinolin-6-yl)oxy)cyclohexyl)carbamate;
cis-3-((1-((2-methylpyrimidin-5-yl)amino)isoquinolin-6-yl)oxy)cyclohexan-1-ol;
(1R,3S)-3-((1-((2-chloropyrimidin-5-yl)amino)isoquinolin-6-yl)oxy)cyclohexan-1-ol;
(1S,3R)-3-((1-((2-chloropyrimidin-5-yl)amino)isoquinolin-6-yl)oxy)cyclohexan-1-ol;
cis-N-(2-chloropyrimidin-5-yl)-6-(((1S,3R)-3-methoxycyclohexyl)oxy)isoquinolin-1-amine;
1-(((1-((2-chloropyrimidin-5-yl)amino)isoquinolin-6-yl)oxy)methyl)cyclopropane-1-carboxamide;
cis-3-(((1-((2-chloropyrimidin-5-yl)amino)isoquinolin-6-yl)oxy)methyl)-1-iminohexahydro-1λ6-thiopyran 1-oxide;
trans-3-(((1-((2-chloropyrimidin-5-yl)amino)isoquinolin-6-yl)oxy)methyl)-1-iminohexahydro-1λ6-thiopyran 1-oxide;
1-(((1-((2-chloropyrimidin-5-yl)amino)isoquinolin-6-yl)oxy)methyl)cyclobutane-1-carbonitrile;
N-(2-methoxypyrimidin-5-yl)-6-((3-methyloxetan-3-yl)methoxy)isoquinolin-1-amine;
N-(2-methoxypyrimidin-5-yl)-6-((1-methyl-1H-pyrazol-4-yl)methoxy)isoquinolin-1-amine;
6-(cyclopropylmethoxy)-N-(2-methoxypyrimidin-5-yl)isoquinolin-1-amine;
1-(((1-((2-methoxypyrimidin-5-yl)amino)isoquinolin-6-yl)oxy)methyl)cyclopropane-1-carbonitrile;
N-((1R,3S)-3-((1-((2-chloropyrimidin-5-yl)amino)isoquinolin-6-yl)oxy)cyclohexyl)-2,2,2-trifluoroacetamide;
N-((1R,3S)-3-((1-((2-chloropyrimidin-5-yl)amino)isoquinolin-6-yl)oxy)cyclohexyl)acetamide;
N-((1S,3R)-3-((1-((2-chloropyrimidin-5-yl)amino)isoquinolin-6-yl)oxy)cyclohexyl)-2,2,2-trifluoroacetamide;
N-((1S,3R)-3-((1-((2-chloropyrimidin-5-yl)amino)isoquinolin-6-yl)oxy)cyclohexyl)acetamide;
6-((1H-pyrazol-4-yl)methoxy)-N-(2-chloropyrimidin-5-yl)isoquinolin-1-amine;
(R)-6-((1,4-dioxan-2-yl)methoxy)-N-(2-chloropyrimidin-5-yl)isoquinolin-1-amine;
(S)-6-((1,4-dioxan-2-yl)methoxy)-N-(2-chloropyrimidin-5-yl)isoquinolin-1-amine;
N-(2-chloropyrimidin-5-yl)-6-((1-fluorocyclopropyl)methoxy-d2)isoquinolin-1-amine;
1-(1-((1-((2-chloropyrimidin-5-yl)amino)isoquinolin-6-yl)oxy)ethyl)cyclopropane-1-carbonitrile;
N-(2-methoxypyrimidin-5-yl)-6-((5-methyl-1H-pyrazol-4-yl)methoxy)isoquinolin-1-amine;
6-(3-(1H-pyrazol-4-yl)propoxy)-N-(2-chloropyrimidin-5-yl)isoquinolin-1-amine;
6-(2-(1H-pyrazol-4-yl)ethoxy)-N-(2-chloropyrimidin-5-yl)isoquinolin-1-amine;
6-((1H-pyrazol-4-yl)methoxy)-N-(2-methylpyrimidin-5-yl)isoquinolin-1-amine;
6-((3-methyl-1H-pyrazol-4-yl)methoxy)-N-(2-methylpyrimidin-5-yl)isoquinolin-1-amine; and
N-(2-chloropyrimidin-5-yl)-6-((5,5-dimethyltetrahydrofuran-2-yl)methoxy)isoquinolin-1-amine;
as an individual stereoisomer, enantiomer or tautomer thereof or a mixture thereof;
or a pharmaceutically acceptable salt, solvate or prodrug thereof.

12-14. (canceled)

15. The compound of claim 1 wherein Y is ═C(R⁵)— having the formula (Ib1) or formula (Ic1): 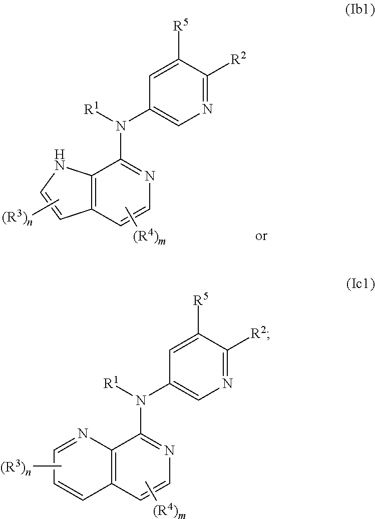
wherein n is 0, 1 or 2 and m, R¹, R², R³, R⁴ and R⁵ are each as described above in claim 1;
as a stereoisomer, enantiomer or tautomer thereof or a mixture thereof, or a pharmaceutically acceptable salt, solvate or prodrug thereof.

16-17. (canceled)

18. The compound of claim 1 selected from the following:
N-(6-chloropyridin-3-yl)-1H-pyrrolo[2,3-c]pyridin-7-amine;
N-(6-chloropyridin-3-yl)-4-methoxy-1H-pyrrolo[2,3-c]pyridin-7-amine;
N-(6-chloropyridin-3-yl)-1,7-naphthyridin-8-amine; and
N-(6-chloropyridin-3-yl)-4-methyl-1H-pyrrolo[2,3-c]pyridin-7-amine;
N⁸-(6-chloropyridin-3-yl)-N³-((1-methyl-1H-pyrazol-4-yl)methyl)-1,7-naphthyridine-3,8-diamine;
N-(6-chloropyridin-3-yl)-3-((1-methyl-1H-pyrazol-4-yl)methoxy)-1,7-naphthyridin-8-amine;
N-(6-chloropyridin-3-yl)-3-(cyclopropylmethoxy)-1,7-naphthyridin-8-amine;
3-(cyclopropylmethoxy)-N-(6-methylpyridin-3-yl)-1,7-naphthyridin-8-amine;
N-(6-chloropyridin-3-yl)-3-((3-methyloxetan-3-yl)methoxy)-1,7-naphthyridin-8-amine;
N-(6-chloropyridin-3-yl)-3-methoxy-1,7-naphthyridin-8-amine;
N⁷-(6-chloropyridin-3-yl)-N⁴-(2,2,2-trifluoroethyl)-1H-pyrrolo[2,3-c]pyridine-4,7-diamine; and
N⁴-benzyl-N⁷-(6-chloropyridin-3-yl)-1H-pyrrolo[2,3-c]pyridine-4,7-diamine, as an individual stereoisomer, enantiomer or tautomer thereof or a mixture thereof;
or a pharmaceutically acceptable salt, solvate or prodrug thereof.

19. The compound of claim 1 wherein Y is ═N— having one of the following formula (Ib2) or formula (Ic2): 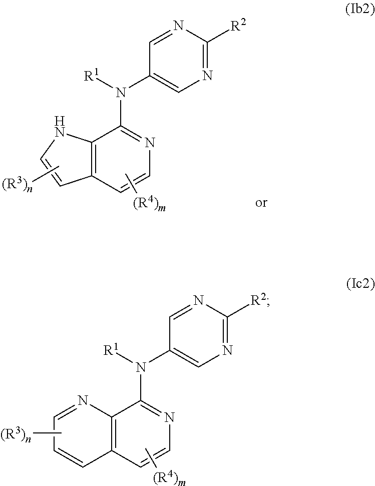
wherein n is 0, 1 or 2 and m, R¹, R², R³ and R⁴ are each as described above in claim 1;
as a stereoisomer, enantiomer or tautomer thereof or a mixture thereof; or a pharmaceutically acceptable salt, solvate or prodrug thereof.

20-21. (canceled)

22. The compound of claim 1 is a compound of Formula (I) selected from the following:
N-(2-chloropyrimidin-5-yl)-1,7-naphthyridin-8-amine;
N-(2-chloropyrimidin-5-yl)-3-((1-methyl-1H-pyrazol-4-yl)methoxy)-1,7-naphthyridin-8-amine;
1-(((8-((2-chloropyrimidin-5-yl)amino)-1,7-naphthyridin-3-yl)oxy)methyl)cyclopropane-1-carbonitrile;
3-(cyclopropylmethoxy)-N-(2-methylpyrimidin-5-yl)-1,7-naphthyridin-8-amine; and
3-((1-methyl-1H-pyrazol-4-yl)methoxy)-N-(2-methylpyrimidin-5-yl)-1,7-naphthyridin-8-amine;
as an individual stereoisomer, enantiomer or tautomer thereof or a mixture thereof; or a pharmaceutically acceptable salt, solvate or prodrug thereof.

23. (canceled)

24. The compound of claim 1 wherein Y is ═C(R⁵)— having one of the following formula (Id1) or formula (Ie1): 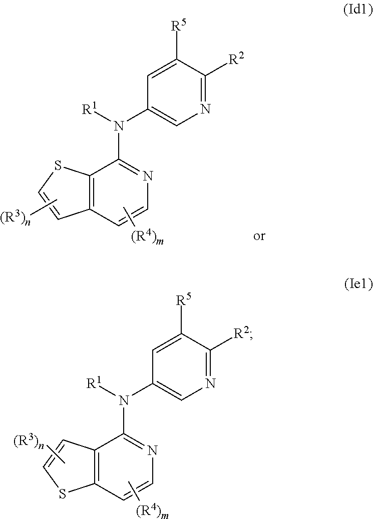
wherein n is 0, 1 or 2 and m, R¹, R², R³, R⁴ and R⁵ are each as described above in claim 1;
as a stereoisomer, enantiomer or tautomer thereof or a mixture thereof, or a pharmaceutically acceptable salt, solvate or prodrug thereof.

25-26. (canceled)

27. The compound of claim 1 selected from the following:
N-(6-chloropyridin-3-yl)-3-methylthieno[2,3-c]pyridin-7-amine;
N-(6-chloropyridin-3-yl)thieno[2,3-c]pyridin-7-amine; and
N-(6-chloropyridin-3-yl)thieno[3,2-c]pyridin-4-amine;
as an individual stereoisomer, enantiomer or tautomer thereof or a mixture thereof, or a pharmaceutically acceptable salt, solvate or prodrug thereof.

28-29. (canceled)

30. The compound of claim 1 wherein Y is ═C(R⁵)— having one of the following formula (If1) or formula (Ig1): 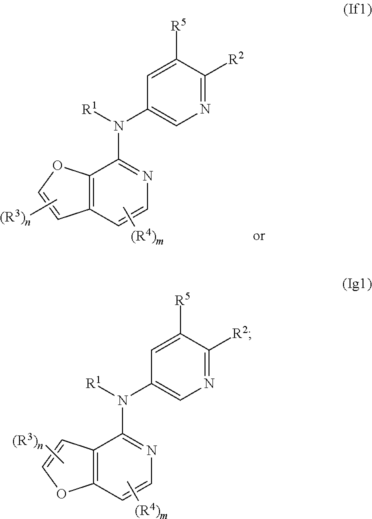
wherein n is 0, 1 or 2 and m, R¹, R², R³, R⁴ and R⁵ are each as described above in claim 1;
as a stereoisomer, enantiomer or tautomer thereof or a mixture thereof, or a pharmaceutically acceptable salt, solvate or prodrug thereof.

31-32. (canceled)

33. The compound of claim 1 selected from the following:
N-(6-chloropyridin-3-yl)furo[2,3-c]pyridin-7-amine; and
N-(6-chloropyridin-3-yl)furo[3,2-c]pyridin-4-amine;
as an individual stereoisomer, enantiomer or tautomer thereof or a mixture thereof, or a pharmaceutically acceptable salt, solvate or prodrug thereof.

34. (canceled)

35. The compound of claim 1 wherein [symbol] is S,N-heteroaryl having the following formula (Ih): 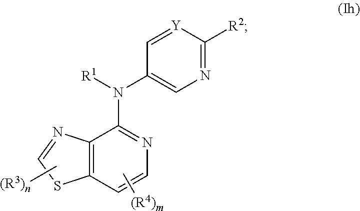
wherein n is 0 or 1 and m, Y, R¹, R², R³ and R⁴ are each as described above in claim 1;
as a stereoisomer, enantiomer or tautomer thereof or a mixture thereof, or a pharmaceutically acceptable salt, solvate or prodrug thereof.

36. The compound of claim 35 wherein Y is ═C(R⁵)— having the following formula (Ih1): 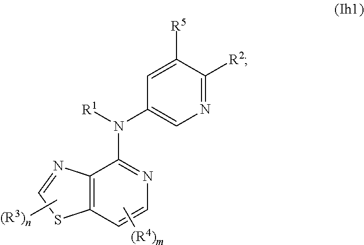
wherein n is 0 or 1 and m, n, R¹, R², R³, R⁴ and R⁵ are as described above in claim 1;
as a stereoisomer, enantiomer or tautomer thereof or a mixture thereof, or a pharmaceutically acceptable salt, solvate or prodrug thereof.

37-38. (canceled)

39. The compound of claim 1 that is N-(6-chloropyridin-3-yl)thiazolo[4,5-c]pyridin-4-amine; as an individual stereoisomer, enantiomer or tautomer thereof or a mixture thereof, or a pharmaceutically acceptable salt, solvate or prodrug thereof.

40. The compound of claim 35 wherein Y is ═N— having the following formula (Ih2): 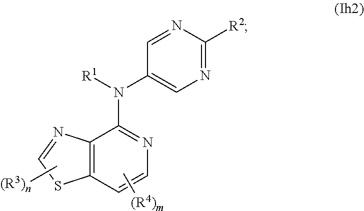
wherein n is 0 or 1 and m, R¹, R², R and R⁴ are each as described above in claim 1;
as a stereoisomer, enantiomer or tautomer thereof or a mixture thereof, or a pharmaceutically acceptable salt, solvate or prodrug thereof.

41. A pharmaceutical composition comprising a pharmaceutically acceptable excipient and a compound of Formula (I): 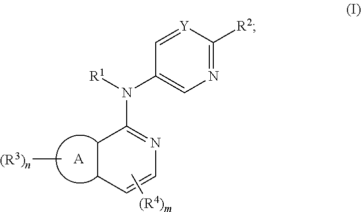
wherein:
m is 0 or 1;
n is 0, 1, 2 or 3;
Y is ═C(R⁵)— or ═N—; [symbol]
is a fused aryl or fused heteroaryl;
R¹ is hydrogen, alkyl, alkenyl, alkynyl, cycloalkyl, cycloalkylalkyl, aralkyl, heterocyclylalkyl or heteroarylalkyl;
R² is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
or R² and Y form a fused 5-membered cycloalkyl, a fused 5-membered heterocyclyl or a fused 5-membered heteroaryl;
each R³ is independently alkyl, alkenyl, alkynyl, halo, haloalkyl, haloalkenyl, cyanoalkyl, —R⁹—OR⁶, —R⁹—N(R⁶)₂, —R⁹—C(O)R⁶, —R⁹—C(O)OR⁶, —R⁹—C(O)N(R⁶)₂, —N═S(O)(R⁷)R, cycloalkyl, cycloalkylalkyl, aryl, aralkyl, heterocyclyl, heterocyclylalkyl, heteroaryl or heteroarylalkyl;
each R⁴ is independently —R⁹—OR⁶, —R⁹—N(R⁶)₂, —R⁹—C(O)R⁶, —R⁹—C(O)OR⁶, —R⁹—C(O)N(R⁶)₂, halo, alkyl, alkenyl, alkynyl, haloalkyl, haloalkenyl; cycloalkyl, cycloalkylalkyl, aryl, aralkyl, heterocyclyl, heterocyclylalkyl, heteroaryl or heteroarylalkyl;
R⁵ is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aralkyl, heterocyclylalkyl or heteroarylalkyl;
each R⁶ is independently hydrogen, —R¹⁰—OR¹¹, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R⁹—OR⁶, cycloalkyl, cycloalkylalkyl, heterocyclyl, heterocyclylalkyl, aryl or aralkyl;
each R⁹ is independently a direct bond, a straight or branched alkylene chain or a straight or branched alkenylene chain;
R¹⁰ is a straight or branched alkylene chain or a straight or branched alkenylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl;
as a stereoisomer, enantiomer or tautomer thereof or a mixture thereof, or a pharmaceutically acceptable salt, solvate or prodrug thereof.

42. A method of treating a disease or condition in a mammal modulated by a voltage-gated potassium channel, wherein the method comprises administering to a mammal in need thereof a therapeutically effective amount of a compound of Formula (I): 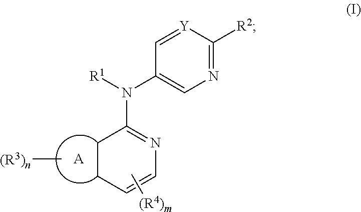
wherein:
m is 0 or 1;
n is 0, 1, 2 or 3;
Y is ═C(R⁵)— or ═N—; [symbol]
is a fused aryl or fused heteroaryl;
R¹ is hydrogen, alkyl, alkenyl, alkynyl, cycloalkyl, cycloalkylalkyl, aralkyl, heterocyclylalkyl or heteroarylalkyl;
R² is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
or R² and Y form a fused 5-membered cycloalkyl, a fused 5-membered heterocyclyl or a fused 5-membered heteroaryl;
each R³ is independently alkyl, alkenyl, alkynyl, halo, haloalkyl, haloalkenyl, cyanoalkyl, —R⁹—OR⁶, —R⁹—N(R⁶)₂, —R⁹—C(O)R⁶, —R⁹—C(O)OR⁶, —R⁹—C(O)N(R⁶)₂, —N═S(O)(R⁷)R, cycloalkyl, cycloalkylalkyl, aryl, aralkyl, heterocyclyl, heterocyclylalkyl, heteroaryl or heteroarylalkyl;
each R⁴ is independently —R⁹—OR⁶, —R⁹—N(R⁶)₂, —R⁹—C(O)R⁶, —R⁹—C(O)OR⁶, —R⁹—C(O)N(R⁶)₂, halo, alkyl, alkenyl, alkynyl, haloalkyl, haloalkenyl; cycloalkyl, cycloalkylalkyl, aryl, aralkyl, heterocyclyl, heterocyclylalkyl, heteroaryl or heteroarylalkyl;
R⁵ is hydrogen, —R⁹—OR⁶, —R⁹—N(R⁶)₂, —R⁹—C(O)R⁶, —R⁹—C(O)OR⁶, —R¹⁰—C(O)N(R⁶)₂, halo, alkyl, alkenyl, alkynyl, haloalkyl, haloalkenyl, cycloalkyl, cycloalkylalkyl, aralkyl, heterocyclylalkyl or heteroarylalkyl;
each R⁶ is independently hydrogen, —R¹⁰—OR¹¹, alkyl, alkenyl, alkynyl, haloalkyl, haloalkenyl, cycloalkyl, cycloalkylalkyl, aryl, aralkyl, heterocyclyl, heterocyclylalkyl, heteroaryl or heteroarylalkyl;
R⁷ and R⁸ are each independently alkyl, alkenyl, —R—OR⁶, cycloalkyl, cycloalkylalkyl, heterocyclyl, heterocyclylalkyl, aryl or aralkyl;
each R⁹ is independently a direct bond, a straight or branched alkylene chain or a straight or branched alkenylene chain;
R¹⁰ is a straight or branched alkylene chain or a straight or branched alkenylene chain; and
R¹¹ is hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl or heteroarylalkyl; as a stereoisomer, enantiomer or tautomer thereof or a mixture thereof; or a pharmaceutically acceptable salt, solvate or prodrug thereof.
